(19) United States
(12) Patent Application Publication
(10) Pub. No.: US 2023/0345816 A1
(43) Pub. Date: Oct. 26, 2023

(54) COMPOUND, ORGANIC ELECTROLUMINESCENCE ELEMENT, AND ELECTRONIC DEVICE

(71) Applicant: IDEMITSU KOSAN CO.,LTD., Tokyo (JP)

(72) Inventors: Yuki NAKANO, Tokyo (JP); Taro YAMAKI, Tokyo (JP); Hiroaki ITOI, Tokyo (JP); Takamoto MORITA, Tokyo (JP); Tomoki KATO, Tokyo (JP); Nozomi TAJIMA, Tokyo (JP); Satomi TASAKI, Tokyo (JP); Kazuki NISHIMURA, Tokyo (JP)

(73) Assignee: IDEMITSU KOSAN CO.,LTD., Tokyo (JP)

(21) Appl. No.: 17/779,911

(22) PCT Filed: Nov. 26, 2020
(86) PCT No.: PCT/JP2020/044062

§ 371 Date: May 25, 2022

(30) Foreign Application Priority Data
Nov. 26, 2019 (JP) 2019-213374
Dec. 27, 2019 (JP) 2019-239596
Dec. 27, 2019 (JP) 2019-239821
Dec. 27, 2019 (JP) 2019-239883
Dec. 27, 2019 (JP) 2019-239910
Dec. 27, 2019 (JP) 2019-239923
Dec. 27, 2019 (JP) 2019-239931
Dec. 27, 2019 (JP) 2019-239937
Feb. 14, 2020 (JP) 2020-023551
Apr. 15, 2020 (JP) 2020-072968
Apr. 15, 2020 (JP) 2020-072969

(51) Int. Cl. H10K 85/60; C07C 15/38; C09K 11/06

(52) U.S. Cl. CPC H10K 85/626; C07C 15/38; C09K 11/06; H10K 85/622; H10K 50/121

(57) ABSTRACT

A compound represented by the formula (12X). In the formula (12X), Py₁ and Py₂ each independently represent a substituted or unsubstituted 1-pyrenyl group, L₁ and L₂ each independently represent a substituted or unsubstituted phenylene group or a substituted or unsubstituted naphthylene group. When L₁ and L₂ each independently represent a substituted or unsubstituted phenylene group, -L₁-L₂- in the formula (12X) represents a group represented by one of the formulae (13-1) to (13-6), (10-1), (20-1), and (30-1). When L₁ and L₂ each independently represent a substituted or unsubstituted naphthylene group, a bonding position of the naphthylene group in L₁ is different from a bonding position of the naphthylene group in L₂.

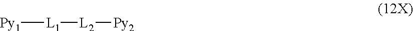

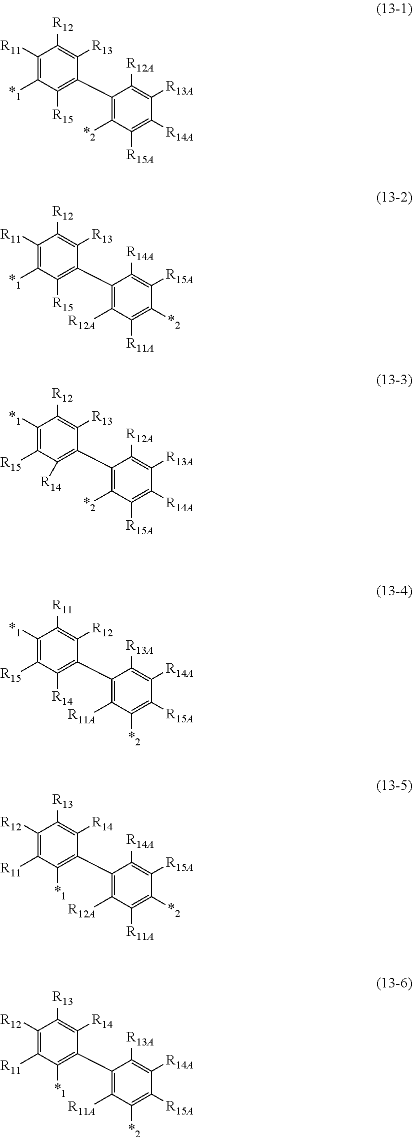

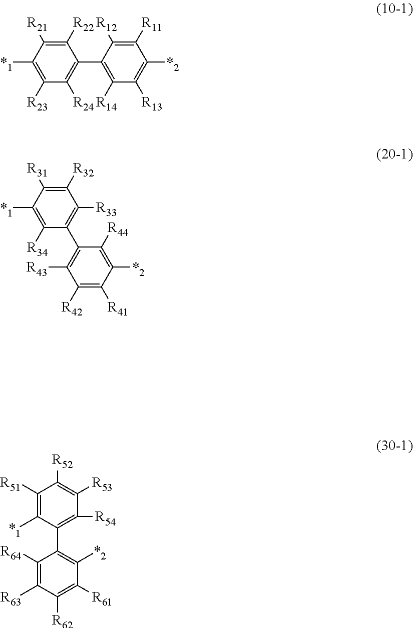

### TECHNICAL FIELD

The present invention relates to a compound, an organic electroluminescence device, and an electronic device.

### BACKGROUND ART

Organic electroluminescence devices (hereinafter sometimes referred to as “organic EL devices”) are applied to full-color displays of mobile phones, television sets, and the like. Upon the application of a voltage to an organic EL device, holes are injected into an emitting layer from an anode, and electrons are injected into the emitting layer from a cathode. The holes and electrons injected into the emitting layer recombine and form excitons. According to the statistical law of electron spins, singlet excitons are generated at a rate of 25%, and triplet excitons are generated at a rate of 75%.

To improve the performance of organic EL devices, various studies have been made on compounds used for the organic EL devices (see Patent Literature 1 to Patent Literature 6, for example). The performance of organic EL devices includes luminance, emission wavelength, chromaticity, luminous efficiency, drive voltage, and life, for example.

### CITATION LIST

### Patent Literatures

Patent Literature 1: JP 2013-157552 A
Patent Literature 2: International Publication No. WO 2004/018587
Patent Literature 3: International Publication No. WO 2005/115950
Patent Literature 4: International Publication No. WO 2011/077691
Patent Literature 5: JP 2018-125504 A
Patent Literature 6: U.S. Patent Application Publication No. 2019/280209

### SUMMARY OF THE INVENTION

### Problems to be Solved by the Invention

An object of the invention is to provide a compound that can improve luminous efficiency, an organic EL device produced using the compound, and an electronic device including the organic EL device.

Another object of the invention is to provide an organic electroluminescence device with improved performance. Still another object of the invention is to provide an organic electroluminescence device with improved luminous efficiency and an electronic device including the organic electroluminescence device.

### Means for Solving the Problems

An aspect of the invention provides a compound represented by the following formula (12X).

[Formula 1]

Py₁-L₁-L₂-Py₂ (12X)

In the formula (12X):
Py₁ and Py₂ each independently represent a substituted or unsubstituted 1-pyrenyl group;
L₁ and L₂ each independently represent a substituted or unsubstituted phenylene group, or a substituted or unsubstituted naphthylene group;
when L₁ and L₂ each independently represent a substituted or unsubstituted phenylene group, -L₁-L₂- in the formula (12X) represents a group represented by one of the following formulae (13-1) to (13-6), (10-1), (20-1), and (30-1), and when L₁ and L₂ each independently represent a substituted or unsubstituted naphthylene group, a bonding position of the naphthylene group as L₁ is different from a bonding position of the naphthylene group as L₂;
when Py₁ and Py₂ each independently represent a substituted 1-pyrenyl group, a substituent E of the substituted 1-pyrenyl group each independently represent a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms; and
when a plurality of substituents E are present, the plurality of substituents E are mutually the same or different.

A substituent F for “substituted or unsubstituted” in the substituent E is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

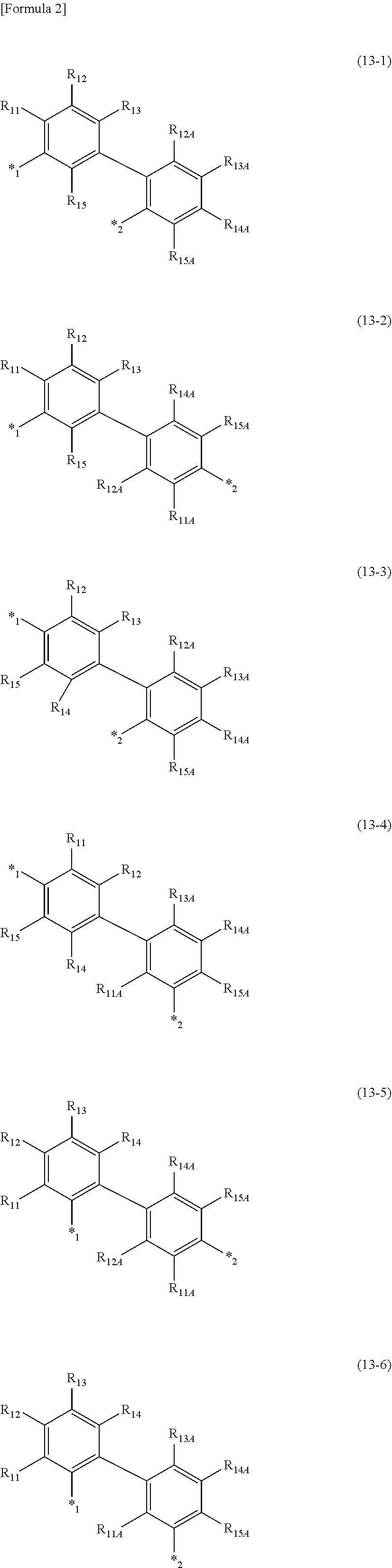

In the formulae (13-1) to (13-6), R₁₁ to R₁₅ and R_{11A} to R_{15A} each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms;
a substituent for “substituted or unsubstituted” in R₁₁ to R₁₅ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group;
at least one combination of adjacent two or more of R₁₁ to R₁₅ are not mutually bonded to form no substituted or unsubstituted monocyclic ring or to form no substituted or unsubstituted fused ring;
at least one combination of adjacent two or more of R_{11A} to R_{15A} are not mutually bonded to form no substituted or unsubstituted monocyclic ring or to form no substituted or unsubstituted fused ring; and
*1 in the formulae (13-1) to (13-6) represents a position of bonding to Py₁ in the formula (12X), and *2 represents a position of bonding to Py₂ in the formula (12X).

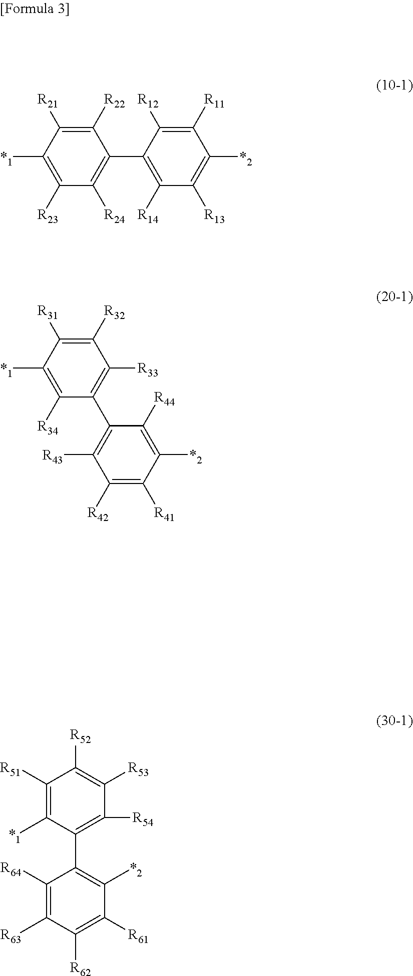

In the formula (10-1):
at least one combination of a combination of R₁₁ and R₁₂, a combination of R₁₃ and R₁₄, a combination of R₂₁ and R₂₂, or a combination of R₂₃ and R₂₄ are mutually bonded to form a substituted or unsubstituted monocyclic ring, are mutually bonded to form a substituted or unsubstituted fused ring, or are not mutually bonded;
R₁₁, R₁₃, R₂₁, and R₂₃ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4);
R₁₂, R₁₄, R₂₂, and R₂₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4);
at least one of R₁₁ to R₁₄ or R₂₁ to R₂₄ is not a hydrogen atom.

In the formulae (20-1) and (30-1):
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring, are mutually bonded to form a substituted or unsubstituted fused ring, or are not mutually bonded;
R₃₁ to R₃₄, R₄₁ to R₄₄, R₅₁ to R₅₄, and R₆₁ to R₆₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted spirobifluorenyl group, or a group represented by the following formula (4);
at least one of R₃₁ to R₃₄ or R₄₁ to R₄₄ is not a hydrogen atom, and
at least one of R₅₁ to R₅₄ or R₆₁ to R₆₄ is not a hydrogen atom.

In a compound represented by the formula (10-1), a combination of R₁₁ and R₁₃ and a combination of R₂₁ and R₂₃ are different combinations, or a combination of R₁₂ and R₁₄ and a combination of R₂₂ and R₂₄ are different combinations.

In a compound represented by the formula (20-1), R₃₁ is different from R₄₁, R₃₂ is different from R₄₂, R₃₃ is different from R₄₃, or R₃₄ is different from R₄₄.

In a compound represented by the formula (30-1), R₅₁ is different from R₆₁, R₅₂ is different from R₆₂, R₅₃ is different from R₆₃, or R₅₄ is different from R₆₄.
*1 in the formulae (10-1), (20-1), and (30-1) represents a position of bonding to Py₁ in the formula (12X), and *2 represents a position of bonding to Py₂ in the formula (12X).

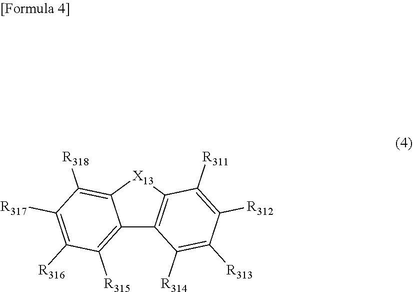

In the formula (4):
X₁₃ represents an oxygen atom, a sulfur atom, or NR₃₁₉;
at least one combination of adjacent two or more of R₃₁₁ to R₃₁₈ are mutually bonded to form a substituted or unsubstituted monocyclic ring, are mutually bonded to form a substituted or unsubstituted fused ring, or are not mutually bonded;
R₃₁₁ to R₃₁₉ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group; and
any one of R₃₁₁ to R₃₁₉ that do not form the substituted or unsubstituted monocyclic ring and do not form the substituted or unsubstituted fused ring is a single bond bonded to at least one of R₁₁ to R₁₄, R₂₁ to R₂₄, R₃₁ to R₃₄, R₄₁ to R₄₄, R₅₁ to R₅₄, Or R₆₁ to R₆₄.

In the formulae (10-1), (20-1), and (30-1) and the formula (4), a substituent for “substituted or unsubstituted” in R₁₁ to R₁₄, R₂₁ to R₂₄, R₃₁ to R₃₄, R₄₁ to R₄₄, R₅₁ to R₅₄, R₆₁ to R₆₄, and R₃₁₁ to R₃₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

An aspect of the invention provides a compound represented by the following formula (120).

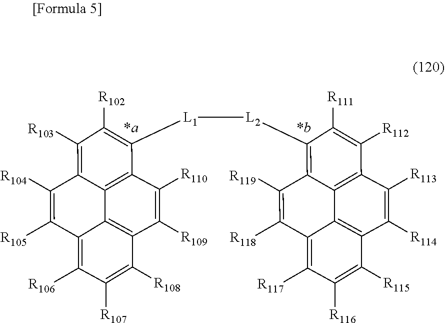

In the formula (120):
L₁ represents a group represented by one of the following formulae (11) to (13); L₂ represents a group represented by one of the following formulae (11A) to (13A);
R₁₀₂ to R₁₁₉ each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms; and
a substituent for “substituted or unsubstituted” in R₁₀₂ to R₁₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

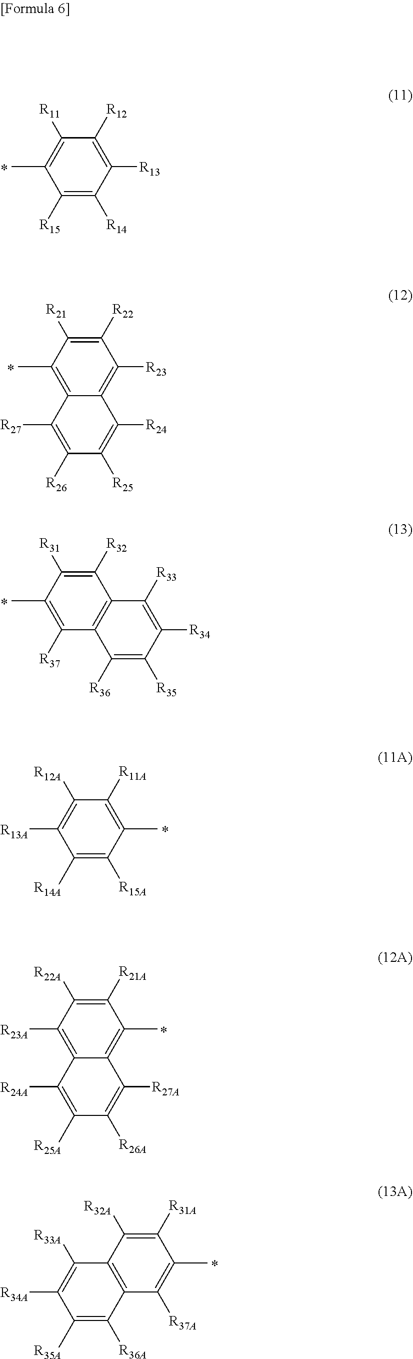

In the formulae (11) to (13) and (11A) to (13A):
R₁₁ to R₁₅, R₂₁ to R₂₇, R₃₁ to R₃₇, R_{11A} to R_{15A}, R_{21A} to R_{27A}, and R_{31A} to R_{37A} each independently represent a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms,
a substituent for “substituted or unsubstituted” in R₁₁ to R₁₅, R₂₁ to R₂₇, and R₃₁ to R₃₇ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group;
at least one combination of adjacent two or more of R₁₁ to R₁₅ are not mutually bonded to form no substituted or unsubstituted monocyclic ring or to form no substituted or unsubstituted fused ring;
at least one combination of adjacent two or more of R_{11A} to R_{15A} are not mutually bonded to form no substituted or unsubstituted monocyclic ring or to form no substituted or unsubstituted fused ring;
at least one combination of adjacent two or more of R₂₁ to R₂₇ are not mutually bonded to form no substituted or unsubstituted monocyclic ring or to form no substituted or unsubstituted fused ring;
at least one combination of adjacent two or more of R_{21A} to R_{27A} are not mutually bonded to form no substituted or unsubstituted monocyclic ring or to form no substituted or unsubstituted fused ring;
at least one combination of adjacent two or more of R₃₁ to R₃₇ are not mutually bonded to form no substituted or unsubstituted monocyclic ring or to form no substituted or unsubstituted fused ring; and
at least one combination of adjacent two or more of R_{31A} to R_{37A} are not mutually bonded to form no substituted or unsubstituted monocyclic ring or to form no substituted or unsubstituted fused ring,
in L₁ represented by the formulae (11) to (13), * represents a position of bonding to *a in the formula (120), and any one of R₁₁ to R₁₅, R₂₁ to R₂₇, and R₃₁ to R₃₇ is a single bond bonded to L₂, and
in L₂ represented by the formulae (11A) to (13A), * represents a position of bonding to *b in the formula (120), and any one of R_{11A} to R_{15A}, R_{21A} to R_{27A}, and R_{31A} to R_{37A} is a single bond bonded to L₁,
when R₁₂ or R₁₄ in L₁ is a single bond bonded to L₂, one of R_{11A}, R_{13A}, R_{15A}, R_{21A} to R_{27A}, and R_{31A} to R_{37A} in L₂ is a single bond bonded to L₁,
when R₁₁ or R₁₅ in L₁ is a single bond bonded to L₂, one of R_{12A}, R_{13A}, R_{14A}, R_{21A} to R_{27A}, and R_{31A} to R_{37A} in L₂ is a single bond bonded to L₁,
when R₁₃ in L₁ is a single bond bonded to L₂, one of R_{11A}, R_{12A}, R_{14A}, R_{15A}, R_{21A} to R_{27A}, and R_{31A} to R_{37A} in L₂ is a single bond bonded to L₁,
when R₂₁ in L₁ is a single bond bonded to L₂, one of R_{11A} to R_{15A}, R_{22A} to R_{27A}, and R_{31A} to R_{37A} in L₂ is a single bond bonded to L₁,
when R₂₂ in L₁ is a single bond bonded to L₂, one of R_{11A} to R_{15A}, R_{21A}, R_{23A} to R_{27A}, and R_{31A} to R_{37A} in L₂ is a single bond bonded to L₁,
when R₂₃ in L₁ is a single bond bonded to L₂, one of R_{11A} to R_{15A}, R_{21A} to R_{22A}, R_{24A} to R_{27A}, and R_{31A} to R_{37A} in L₂ is a single bond bonded to L₁,
when R₂₄ in L₁ is a single bond bonded to L₂, one of R_{11A} to R_{15A}, R_{21A} to R_{23A}, R_{25A} to R_{27A}, and R_{31A} to R_{37A} in L₂ is a single bond bonded to L₁,
when R₂₅ in L₁ is a single bond bonded to L₂, one of R_{11A} to R_{15A}, R_{21A} to R_{24A}, R_{26A} to R_{27A}, and R_{31A} to R_{37A} in L₂ is a single bond bonded to L₁,
when R₂₆ in L₁ is a single bond bonded to L₂, one of R_{11A} to R_{15A}, R_{21A} to R_{25A}, R_{27A}, and R_{31A} to R_{37A} in L₂ is a single bond bonded to L₁,
when R₂₇ in L₁ is a single bond bonded to L₂, one of R_{11A} to R_{15A}, R_{21A} to R_{26A}, and R_{31A} to R_{37A} in L₂ is a single bond bonded to L₁,
when R₃₁ in L₁ is a single bond bonded to L₂, one of R_{11A} to R_{15A}, R_{21A} to R_{27A}, and R_{32A} to R_{37A} in L₂ is a single bond bonded to L₁,
when R₃₂ in L₁ is a single bond bonded to L₂, one of R_{11A} to R_{15A}, R_{21A} to R_{27A}, R_{31A}, and R_{33A} to R_{37A} in L₂ is a single bond bonded to L₁,
when R₃₃ in L₁ is a single bond bonded to L₂, one of R_{11A} to R_{15A}, R_{21A} to R_{27A}, R_{31A} to R_{32A}, and R_{34A} to R_{37A} in L₂ is a single bond bonded to L₁,
when R₃₄ in L₁ is a single bond bonded to L₂, one of R_{11A} to R_{15A}, R_{21A} to R_{27A}, R_{31A} to R_{33A}, and R_{35A} to R_{37A} in L₂ is a single bond bonded to L₁,
when R₃₅ in L₁ is a single bond bonded to L₂, one of R_{11A} to R_{15A}, R_{21A} to R_{27A}, R_{31A} to R_{34A}, and R_{36A} to R_{37A} in L₂ is a single bond bonded to L₁,
when R₃₆ in L₁ is a single bond bonded to L₂, one of R_{11A} to R_{15A}, R_{21A} to R_{27A}, R_{31A} to R_{35A}, and R_{37A} in L₂ is a single bond bonded to L₁, and
when R₃₇ in L₁ is a single bond bonded to L₂, one of R_{11A} to R_{15A}, R_{21A} to R_{27A}, and R_{31A} to R_{36A} in L₂ is a single bond bonded to L₁.

An aspect of the invention provides a compound represented by the following formula (1), (2), or (3).

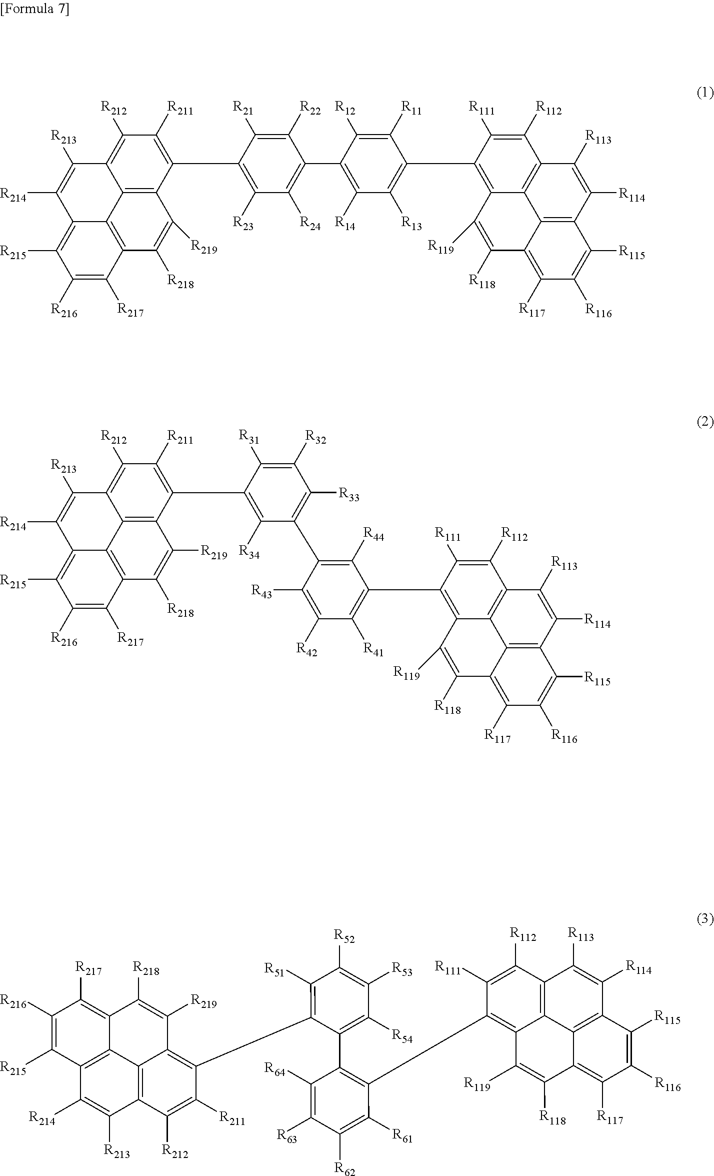

In the formulae (1) to (3):
R₁₁₁ to R₁₁₉ and R₂₁₁ to R₂₁₉ each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms; and
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms.

In the formula (1):
at least one combination of a combination of R₁₁ and R₁₂, a combination of R₁₃ and R₁₄, a combination of R₂₁ and R₂₂, or a combination of R₂₃ and R₂₄ are mutually bonded to form a substituted or unsubstituted monocyclic ring, are mutually bonded to form a substituted or unsubstituted fused ring, or are not mutually bonded;
R₁₁, R₁₃, R₂₁, and R₂₃ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4);
R₁₂, R₁₄, R₂₂, and R₂₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4); and
at least one of R₁₁ to R₁₄ or R₂₁ to R₂₄ is not a hydrogen atom.

In the formulae (2) to (3):
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring, are mutually bonded to form a substituted or unsubstituted fused ring, or are not mutually bonded;
R₃₁ to R₃₄, R₄₁ to R₄₄, R₅₁ to R₅₄, and R₆₁ to R₆₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted spirobifluorenyl group, or a group represented by the following formula (4);
at least one of R₃₁ to R₃₄ or R₄₁ to R₄₄ is not a hydrogen atom; and
at least one of R₅₁ to R₅₄ or R₆₁ to R₆₄ is not a hydrogen atom.

In a compound represented by the formula (1), a combination of R₁₁ and R₁₃ and a combination of R₂₁ and R₂₃ are different combinations, or a combination of R₁₂ and R₁₄ and a combination of R₂₂ and R₂₄ are different combinations.

In a compound represented by the formula (2), a combination of R₃₁ and R₄₁ is different from at least one combination of a combination of R₃₂ and R₄₂, a combination of R₃₃ and R₄₃, or a combination of R₃₄ and R₄₄.

In a compound represented by the formula (3), a combination of R₅₁ and R₆₁ is different from at least one combination of a combination of R₅₂ and R₆₂, a combination of R₅₃ and R₆₃, or a combination of R₅₄ and R₆₄.

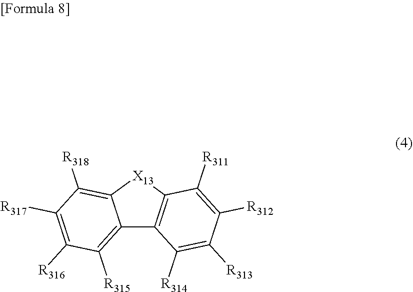

In the formula (4):
X₁₃ represents an oxygen atom, a sulfur atom, or NR₃₁₉,
at least one combination of adjacent two or more of R₃₁₁ to R₃₁₈ are mutually bonded to form a substituted or unsubstituted monocyclic ring, are mutually bonded to form a substituted or unsubstituted fused ring, or are not mutually bonded;
R₃₁₁ to R₃₁₉ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group; and
any one of R₃₁₁ to R₃₁₉ that do not form the substituted or unsubstituted monocyclic ring and do not form the substituted or unsubstituted fused ring is a single bond bonded to at least one of R₁₁ to R₁₄, R₂₁ to R₂₄, R₃₁ to R₃₄, R₄₁ to R₄₄, R₅₁ to R₅₄, or R₆₁ to R₆₄.

In the formulae (1) to (3), a substituent for “substituted or unsubstituted” in R₁₁ to R₁₄, R₂₁ to R₂₄, R₃₁ to R₃₄, R₄₁ to R₄₄, R₅₁ to R₅₄, R₆₁ to R₆₄, R₁₁₁ to R₁₁₉, R₂₁₁ to R₂₁₉, and R₃₁₁ to R₃₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

Another aspect of the invention provides an organic electroluminescence device including an anode, a cathode, an emitting layer between the anode and the cathode, in which the emitting layer contains the compound according to the above aspect of the invention as a host material.

Still another aspect of the invention provides an organic electroluminescence device including an anode, a cathode, a first emitting layer between the anode and the cathode, and a second emitting layer between the first emitting layer and the cathode, wherein the first emitting layer has at least one group represented by the following formula (11) and contains a first compound represented by the following formula (1A) as a first host material, the second emitting layer contains a second compound represented by the following formula (2) as a second host material, and the first emitting layer is in direct contact with the second emitting layer.

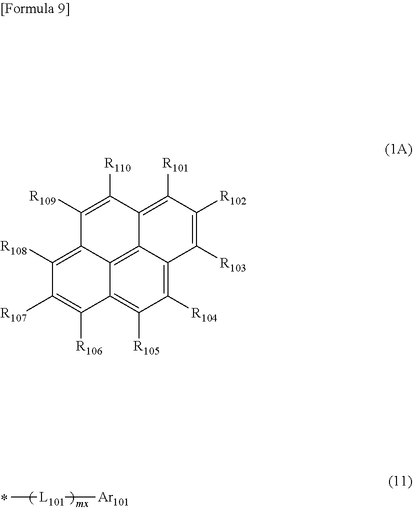

In the formula (1A):
R₁₀₁ to R₁₁₀ each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ represents a group represented by the formula (11);
when a plurality of groups represented by the formula (11) are present, the plurality of groups represented by the formula (11) are mutually the same or different;
L₁₀₁ represents a single bond, a substituted or unsubstituted arylene group having 6 to 50 ring carbon atoms, or a substituted or unsubstituted divalent heterocyclic group having 5 to 50 ring atoms;
Ar₁₀₁ represents a substituted or unsubstituted aryl group having 6 to 50 ring carbon atoms, or a substituted or unsubstituted heterocyclic group having 5 to 50 ring atoms;
mx represents 0, 1, 2, 3, 4, or 5;
when two or more L₁₀₁s are present, the two or more L₁₀₁s are mutually the same or different;
when two or more Ar₁₀₁s are present, the two or more Ar₁₀₁s are mutually the same or different; and
* in the formula (11) represents the position of bonding to a pyrene ring in the formula (1A).

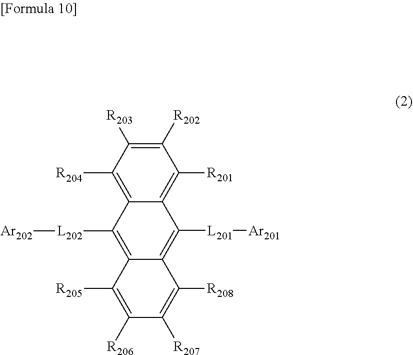

In the formula (2):
R₂₀₁ to R₂₀₈ each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ each independently represent a single bond, a substituted or unsubstituted arylene group having 6 to 50 ring carbon atoms, or a substituted or unsubstituted divalent heterocyclic group having 5 to 50 ring atoms;
Ar₂₀₁ and Ar₂₀₂ each independently represent a substituted or unsubstituted aryl group having 6 to 50 ring carbon atoms, or a substituted or unsubstituted heterocyclic group having 5 to 50 ring atoms.

In the first compound represented by the formula (1A) and the second compound represented by the formula (2):
R₉₀₁, R₉₀₂, R₉₀₃, R₉₀₄, R₉₀₅, R₉₀₆, R₉₀₇, R₈₀₁, and R₈₀₂ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,
when a plurality of R₉₀₁ are present, the plurality of R₉₀₁ are mutually the same or different,
when a plurality of R₉₀₂ are present, the plurality of R₉₀₂ are mutually the same or different,
when a plurality of R₉₀₃ are present, the plurality of R₉₀₃ are mutually the same or different,
when a plurality of R₉₀₄ are present, the plurality of R₉₀₄ are mutually the same or different,
when a plurality of R₉₀₅ are present, the plurality of R₉₀₅ are mutually the same or different,
when a plurality of R₉₀₆ are present, the plurality of R₉₀₆ are mutually the same or different,
when a plurality of R₉₀₇ are present, the plurality of R₉₀₇ are mutually the same or different,
when a plurality of R₈₀₁ are present, the plurality of R₈₀₁ are mutually the same or different, and
when a plurality of R₈₀₂ are present, the plurality of R₈₀₂ are mutually the same or different.

A further aspect of the invention provides an electronic device including the organic electroluminescence device according to the above aspect of the invention.

A further aspect of the invention can provide a compound that can improve luminous efficiency, an organic EL device produced using the compound, and an electronic device including the organic EL device.

The above aspect of the invention can also provide an organic electroluminescence device with improved performance. The above aspect of the invention can also provide an organic electroluminescence device with improved luminous efficiency. The above aspect of the invention can also provide an electronic device including the organic electroluminescence device.

### BRIEF DESCRIPTION OF DRAWINGS

FIG. 1 is a schematic view of an example of an organic electroluminescence device according to a third exemplary embodiment of the invention.

FIG. 2 is a schematic view of an example of an organic electroluminescence device according to a fourth exemplary embodiment of the invention.

### DESCRIPTION OF EMBODIMENTS

### Definition

Herein, hydrogen atoms include isotopes with different numbers of neutrons, that is, protium, deuterium, and tritium.

Herein, in chemical structural formulae, a hydrogen atom, that is, a protium atom, a deuterium atom, or a tritium atom is bonded at a bonding position without symbols, such as “R” or “D”, which represents a deuterium atom.

Herein, ring carbon atoms are carbon atoms among atoms constituting a ring of a compound with a structure in which the atoms are circularly bonded (for example, a monocyclic compound, a fused-ring compound, a cross-linked compound, a carbocyclic compound, or a heterocyclic compound). When the ring is substituted with a substituent, carbons of the substituent are not included in the ring carbon atoms. Unless otherwise specified, the same applies to the “ring carbon atoms” described below. For example, a benzene ring has 6 ring carbon atoms, a naphthalene ring has 10 ring carbon atoms, a pyridine ring has 5 ring carbon atoms, and a furan ring has 4 ring carbon atoms. For example, a 9,9-diphenylfluorenyl group has 13 ring carbon atoms, and a 9,9′-spirobifluorenyl group has 25 ring carbon atoms.

When a benzene ring is substituted, for example, with an alkyl group, carbon atoms of the alkyl group are not included in the ring carbon atoms of the benzene ring. Thus, the benzene ring substituted with the alkyl group has 6 ring carbon atoms. When a naphthalene ring is substituted, for example, with an alkyl group, carbon atoms of the alkyl group are not included in the ring carbon atoms of the naphthalene ring. Thus, the naphthalene ring substituted with the alkyl group has 10 ring carbon atoms.

Herein, ring atoms are atoms constituting a ring of a compound (for example, a monocyclic compound, a fused-ring compound, a cross-linked compound, a carbocyclic compound, or a heterocyclic compound) with a structure in which the atoms are circularly bonded (for example, a monocyclic ring, a fused ring, or polycyclic). Atoms that do not constitute a ring (for example, a hydrogen atom that terminates a bond of an atom constituting the ring) and atoms of a substituent substituting a ring are not included in ring atoms. Unless otherwise specified, the same applies to the following “ring atoms”. For example, a pyridine ring has 6 ring atoms, a quinazoline ring has 10 ring atoms, and a furan ring has 5 ring atoms. For example, the number of hydrogen atoms bonded to a pyridine ring or the number of atoms constituting a substituent is not included in the number of pyridine ring atoms. Thus, a pyridine ring to which a hydrogen atom or a substituent is bonded has 6 ring atoms. For example, the number of hydrogen atoms bonded to carbon atoms of a quinazoline ring or the number of atoms constituting a substituent is not included in the number of quinazoline ring atoms. Thus, a quinazoline ring to which a hydrogen atom or a substituent is bonded has 10 ring atoms.

Herein, “XX to YY carbon atoms” of “a substituted or unsubstituted ZZ group having XX to YY carbon atoms” refers to the carbon atoms of the unsubstituted ZZ group and does not include the carbon atoms of a substituent when substituted. “YY” is larger than “XX”, “XX” represents an integer of 1 or more, and “YY” represents an integer of 2 or more.

Herein, “XX to YY atoms” of “a substituted or unsubstituted ZZ group having XX to YY atoms” refers to the atoms of the unsubstituted ZZ group and does not include the atoms of a substituent when substituted. “YY” is larger than “XX”, “XX” represents an integer of 1 or more, and “YY” represents an integer of 2 or more.

Herein, an unsubstituted ZZ group refers to an “unsubstituted ZZ group” of a “substituted or unsubstituted ZZ group”, and a substituted ZZ group refers to a “substituted ZZ group” of the “substituted or unsubstituted ZZ group”.

Herein, “unsubstituted” of a “substituted or unsubstituted ZZ group” means that a hydrogen atom of the ZZ group is not substituted with a substituent. A hydrogen atom in an “unsubstituted ZZ group” is a protium atom, a deuterium atom, or a tritium atom.

Herein, “substituted” of a “substituted or unsubstituted ZZ group” means that at least one hydrogen atom of the ZZ group is substituted with a substituent. Likewise, “substituted” of a “BB group substituted with an AA group” means that at least one hydrogen atom of the BB group is substituted with the AA group.

“Substituents Mentioned Herein”

Substituents mentioned herein are described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B) below. (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a benzoanthryl group, a phenanthryl group, a benzophenanthryl group, a phenalenyl group, a pyrenyl group, a chrysenyl group, a benzochrysenyl group, a triphenylenyl group, a benzotriphenylenyl group, a tetracenyl group, a pentacenyl group, a fluorenyl group, a 9,9′-spirobifluorenyl group, a benzofluorenyl group, a benzofluorenyl group, a fluoranthenyl group, a benzofluoranthenyl group, a perylenyl group, and a monovalent aryl group derived by removing one hydrogen atom from ring structures represented by the following formulae (TEMP-1) to (TEMP-15).

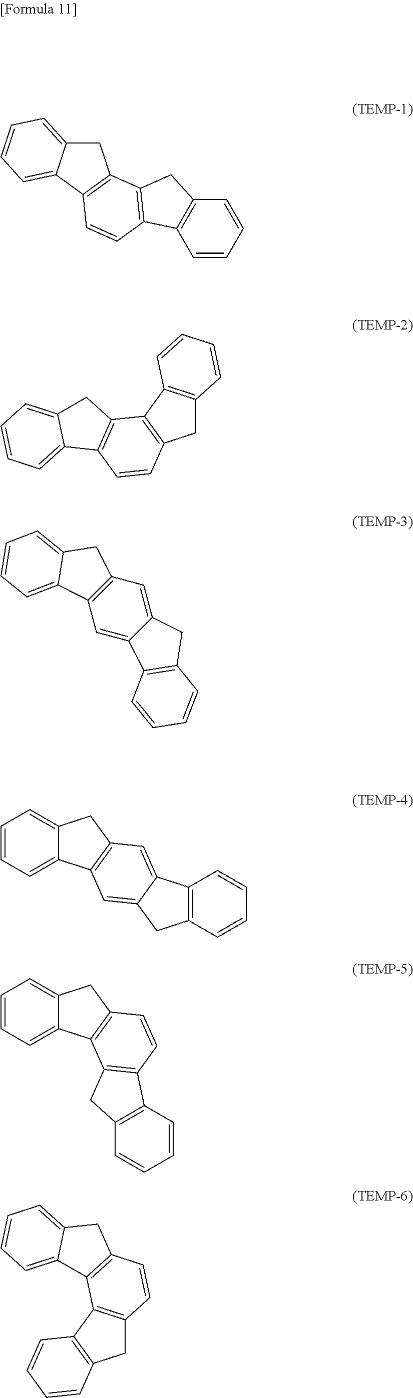

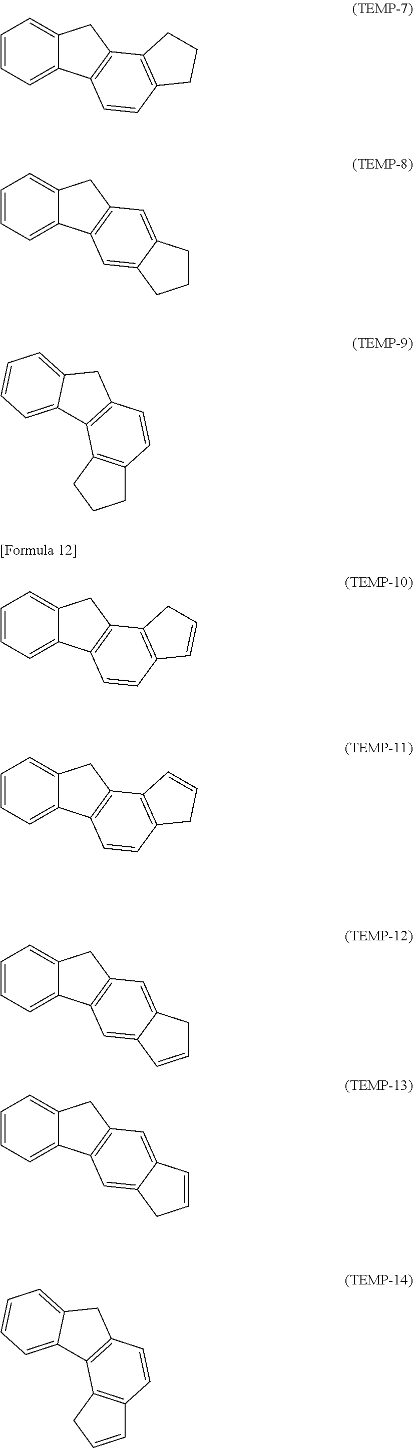

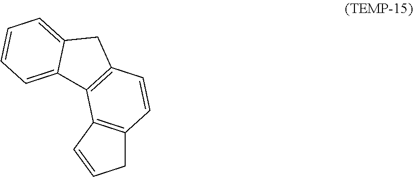 [SMILES: C1=Cc2c(ccc3c2-c2ccccc2C3)C1]

### Substituted Aryl Group (Specific Example Group G1B):

an o-tolyl group, a m-tolyl group, a p-tolyl group, a para-xylyl group, a meta-xylyl group, an ortho-xylyl group, a para-isopropylphenyl group, a meta-isopropylphenyl group, an ortho-isopropylphenyl group, a para-t-butylphenyl group, a meta-t-butylphenyl group, an ortho-t-butylphenyl group, a 3,4,5-trimethylphenyl group, a 9,9-dimethylfluorenyl group, a 9,9-diphenylfluorenyl group, a 9,9-bis(4-methyl phenyl)fluorenyl group, a 9,9-bis(4-isopropylphenyl)fluorenyl group, a 9,9-bis(4-t-butylphenyl)fluorenyl group, a cyanophenyl group, a triphenylsilylphenyl group, a trimethylsilylphenyl group, a phenylnaphthyl group, a naphthylphenyl group, and a group derived by substituting at least one hydrogen atom of a monovalent group derived from the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of “unsubstituted heterocyclic group” and “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

a pyrrolyl group, an imidazolyl group, a pyrazolyl group, a triazolyl group, a tetrazolyl group, an oxazolyl group, an isoxazolyl group, an oxadiazolyl group, a thiazolyl group, an isothiazolyl group, a thiadiazolyl group, a pyridyl group, a pyridazinyl group, a pyrimidinyl group, a pyrazinyl group, a triazinyl group, an indolyl group, an isoindolyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, an indazolyl group, a phenanthrolinyl group, a phenanthridinyl group, an acridinyl group, a phenazinyl group, a carbazolyl group, a benzocarbazolyl group, a morpholino group, a phenoxazinyl group, a phenothiazinyl group, an azacarbazolyl group, and a diazacarbazolyl group.

### Unsubstituted Heterocyclic Groups Including Oxygen Atom (Specific Example Group G2A2):

a furyl group, an oxazolyl group, an isoxazolyl group, an oxadiazolyl group, a xanthenyl group, a benzofuranyl group, an isobenzofuranyl group, a dibenzofuranyl group, a naphthobenzofuranyl group, a benzoxazolyl group, a benzoisoxazolyl group, a phenoxazinyl group, a morpholino group, a dinaphthofuranyl group, an azadibenzofuranyl group, a diazadibenzofuranyl group, an azanaphthobenzofuranyl group, and a diazanaphthobenzofuranyl group.

### Unsubstituted Heterocyclic Groups Including Sulfur Atom (Specific Example Group G2A3):

a thienyl group, a thiazolyl group, an isothiazolyl group, a thiadiazolyl group, a benzothiophenyl group (benzothienyl group), an isobenzothiophenyl group (isobenzothienyl group), a dibenzothiophenyl group (dibenzothienyl group), a naphthobenzothiophenyl group (naphthobenzothienyl group), a benzothiazolyl group, a benzoisothiazolyl group, a phenothiazinyl group, a dinaphthothiophenyl group (dinaphthothienyl group), an azadibenzothiophenyl group (azadibenzothienyl group), a diazadibenzothiophenyl group (diazadibenzothienyl group), an azanaphthobenzothiophenyl group (azanaphthobenzothienyl group), and a diazanaphthobenzothiophenyl group (diazanaphthobenzothienyl group).
Monovalent Heterocyclic Groups Derived by Removing One Hydrogen Atom from Cyclic Structures Represented by Formulae (TEMP-16) to (TEMP-33) (Specific Example Group G2A4):

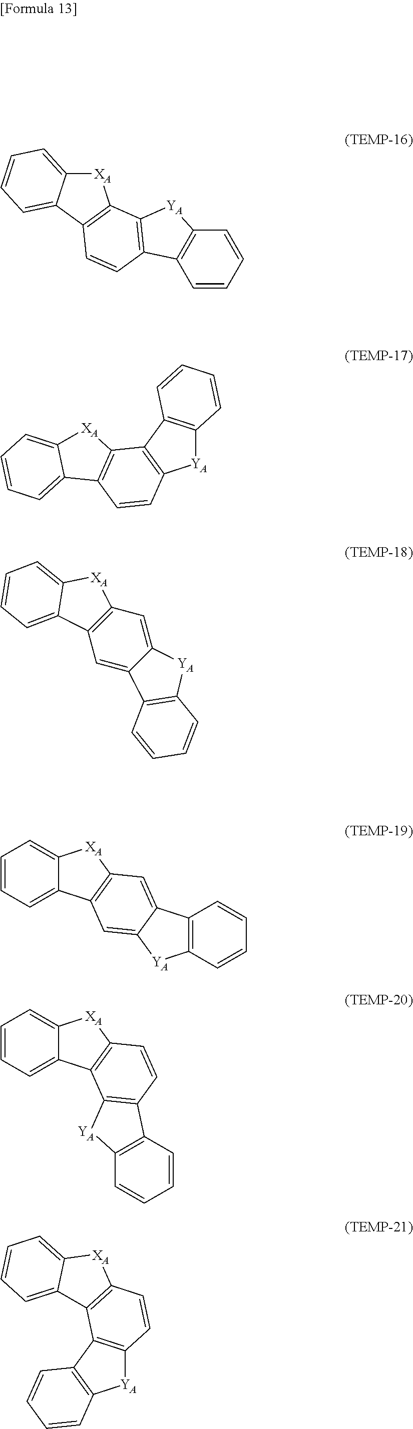

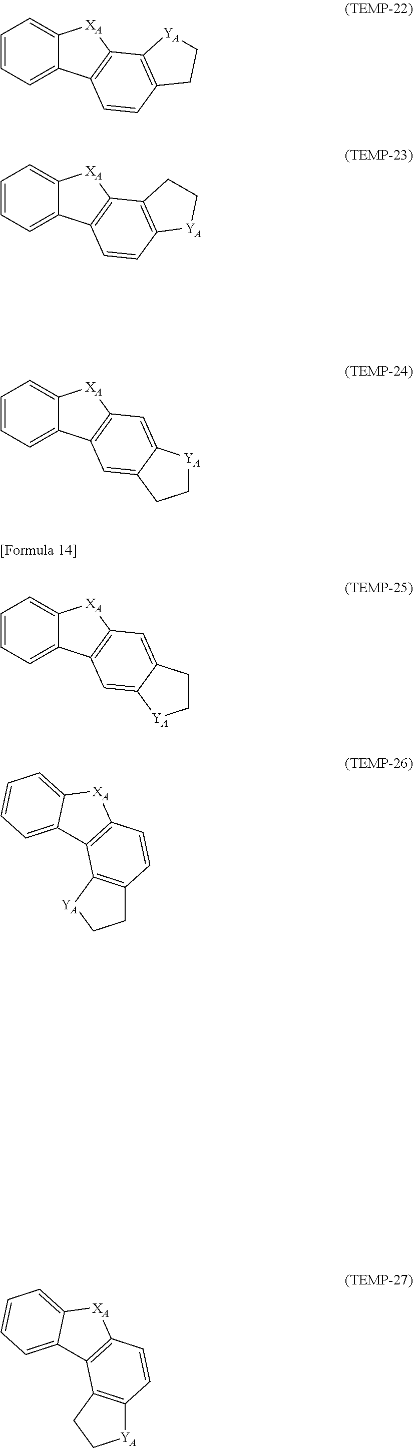

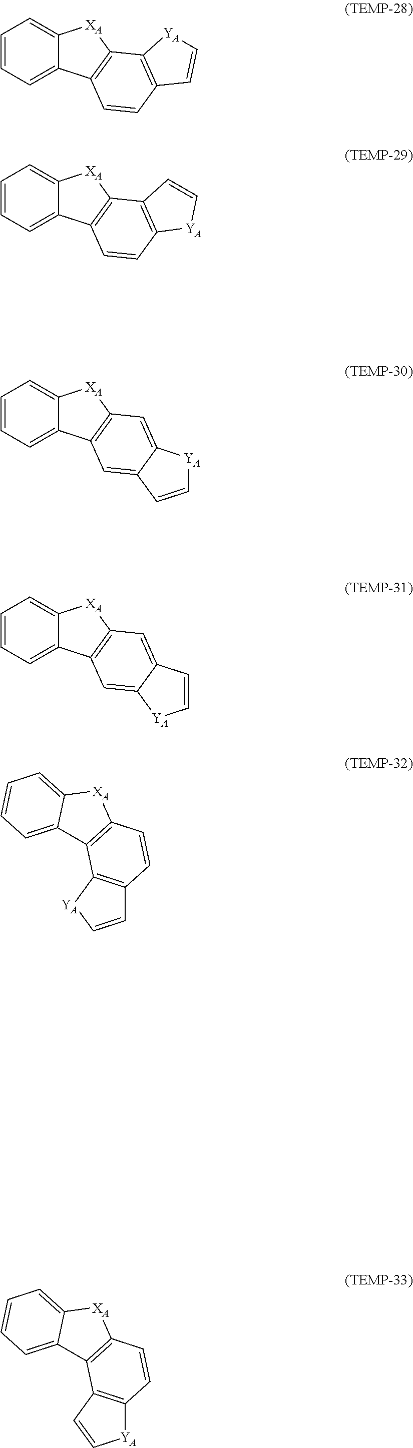

In the formulae (TEMP-16) to (TEMP-33), X_{A} and Y_{A} each independently represent an oxygen atom, a sulfur atom, NH, or CH₂. At least one of X_{A} or Y_{A} represents an oxygen atom, a sulfur atom, or NH.

In the formulae (TEMP-16) to (TEMP-33), when at least one of X_{A} or Y_{A} represents NH or CH₂, a monovalent heterocyclic group derived from the ring structures represented by the formulae (TEMP-16) to (TEMP-33) includes a monovalent group derived by removing one hydrogen atom from the NH or CH₂.

### Substituted Heterocyclic Groups Including Nitrogen Atom (Specific Example Group G2B1):

a (9-phenyl)carbazolyl group, a (9-biphenylyl)carbazolyl group, a (9-phenyl)phenylcarbazolyl group, a (9-naphthyl)carbazolyl group, a diphenylcarbazol-9-yl group, a phenylcarbazol-9-yl group, a methylbenzimidazolyl group, an ethylbenzimidazolyl group, a phenyltriazinyl group, a biphenylyltriazinyl group, a diphenyltriazinyl group, a phenylquinazolinyl group, and a biphenylylquinazolinyl group.

### Substituted Heterocyclic Groups Including Oxygen Atom (Specific Example Group G2B2):

a phenyldibenzofuranyl group, a methyldibenzofuranyl group, a t-butyldibenzofuranyl group, and a monovalent residue of spiro[9H-xanthene-9,9′-[9H]fluorene].

### Substituted Heterocyclic Groups Including Sulfur Atom (Specific Example Group G2B3):

a phenyldibenzothiophenyl group, a methyldibenzothiophenyl group, a t-butyldibenzothiophenyl group, and a monovalent residue of spiro[9H-thioxanthene-9,9′-[9H]fluorene].
Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of “unsubstituted alkyl group” and “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

a methyl group, an ethyl group, a n-propyl group, an isopropyl group, an n-butyl group, an isobutyl group, a s-butyl group, and a t-butyl group.

### Substituted Alkyl Group (Specific Example Group G3B):

a heptafluoropropyl group (including isomers), a pentafluoroethyl group, a 2,2,2-trifluoroethyl group, and a trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of “unsubstituted alkenyl group” and “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, an allyl group, a 1-butenyl group, a 2-butenyl group, and a 3-butenyl group.

### Substituted Alkenyl Group (Specific Example Group G4B):

a 1,3-butanedienyl group, a 1-methylvinyl group, a 1-methylallyl group, a 1,1-dimethylallyl group, a 2-methylallyl group, and a 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A): An Ethynyl Group

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, a cyclobutyl group, a cyclopentyl group, a cyclohexyl group, a 1-adamantyl group, a 2-adamantyl group, a 1-norbornyl group, and a 2-norbornyl group.

### Substituted Cycloalkyl Group (Specific Example Group G6B):

a 4-methylcyclohexyl group.
Group Represented by —Si(R₉₀₁)(R₉₀₂)(R₉₀₃)
Specific examples (specific example group G7) of the group represented herein by —Si(R₉₀₁)(R₉₀₂)(R₉₀₃) include: —Si(G1)(G1)(G1), —Si(G1)(G2)(G2), —Si(G1)(G1)(G2), —Si(G2)(G2)(G2), —Si(G3)(G3)(G3), and —Si(G6)(G6)(G6).
G1 represents a “substituted or unsubstituted aryl group” described in the specific example group G1.
G2 represents a “substituted or unsubstituted heterocyclic group” described in the specific example group G2.
G3 represents a “substituted or unsubstituted alkyl group” described in the specific example group G3.
G6 represents a “substituted or unsubstituted cycloalkyl group” described in the specific example group G6.

A plurality of G1 in —Si(G1)(G1)(G1) are mutually the same or different.

A plurality of G2 in —Si(G1)(G2)(G2) are mutually the same or different.

A plurality of G1 in —Si(G1)(G1)(G2) are mutually the same or different.

A plurality of G2 in —Si(G2)(G2)(G2) are mutually the same or different.

A plurality of G3 in —Si(G3)(G3)(G3) are mutually the same or different.

A plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1), —O(G2), —O(G3), and —O(G6).
G1 represents a “substituted or unsubstituted aryl group” described in the specific example group G1.
G2 represents a “substituted or unsubstituted heterocyclic group” described in the specific example group G2.
G3 represents a “substituted or unsubstituted alkyl group” described in the specific example group G3.
G6 represents a “substituted or unsubstituted cycloalkyl group” described in the specific example group G6.

### Group Represented by —S—(R₉₀₅)

Specific examples (specific example group G9) of a group represented herein by —S—(R₉₀₅) include: —S(G1), —S(G2), —S(G3), and —S(G6).
G1 represents a “substituted or unsubstituted aryl group” described in the specific example group G1.
G2 represents a “substituted or unsubstituted heterocyclic group” described in the specific example group G2.
G3 represents a “substituted or unsubstituted alkyl group” described in the specific example group G3.
G6 represents a “substituted or unsubstituted cycloalkyl group” described in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1), —N(G2)(G2), —N(G1)(G2), —N(G3)(G3), and —N(G6)(G6).
G1 represents a “substituted or unsubstituted aryl group” described in the specific example group G1.
G2 represents a “substituted or unsubstituted heterocyclic group” described in the specific example group G2.
G3 represents a “substituted or unsubstituted alkyl group” described in the specific example group G3.
G6 represents a “substituted or unsubstituted cycloalkyl group” described in the specific example group G6.

A plurality of G1 in —N(G1)(G1) are mutually the same or different.

A plurality of G2 in —N(G2)(G2) are mutually the same or different.

A plurality of G3 in —N(G3)(G3) are mutually the same or different.

A plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, a-naphthylmethyl group, 1-a-naphthylethyl group, 2-a-naphthylethyl group, 1-a-naphthylisopropyl group, 2-a-naphthylisopropyl group, p-naphthylmethyl group, 1-p-naphthylethyl group, 2-p-naphthylethyl group, 1-p-naphthylisopropyl group, and 2-p-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

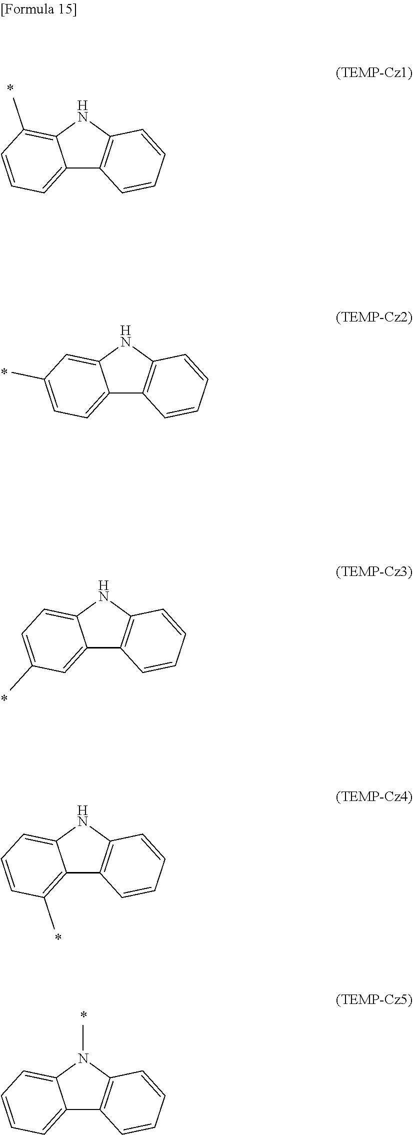

The (9-phenyl)carbazolyl group mentioned herein is, unless otherwise specified herein, specifically a group represented by one of formulae below.

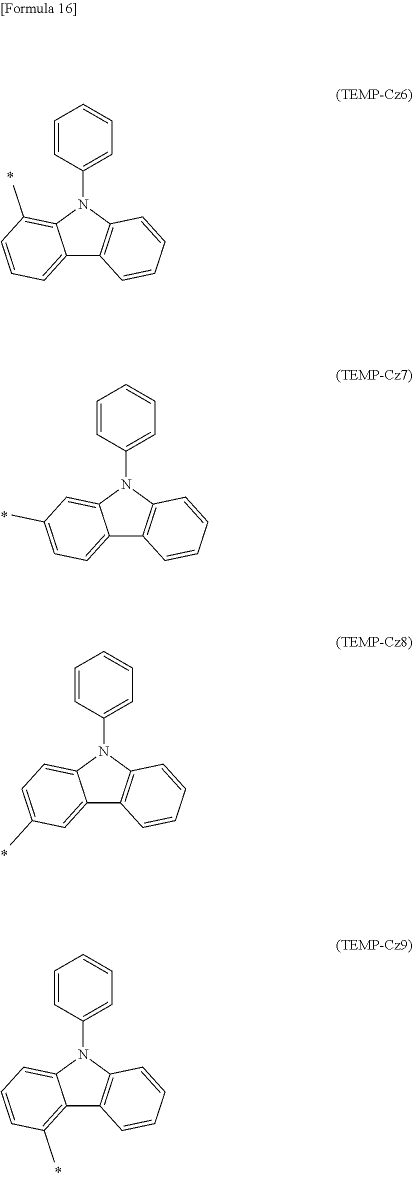 [SMILES: Cc1cccc2c3ccccc3n(-c3ccccc3)c12 ; Cc1ccc2c3ccccc3n(-c3ccccc3)c2c1 ; Cc1ccc2c(c1)c1ccccc1n2-c1ccccc1 ; Cc1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

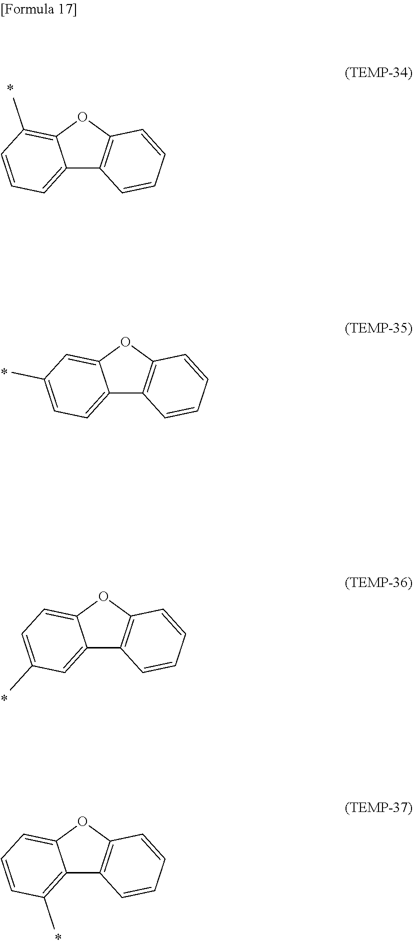

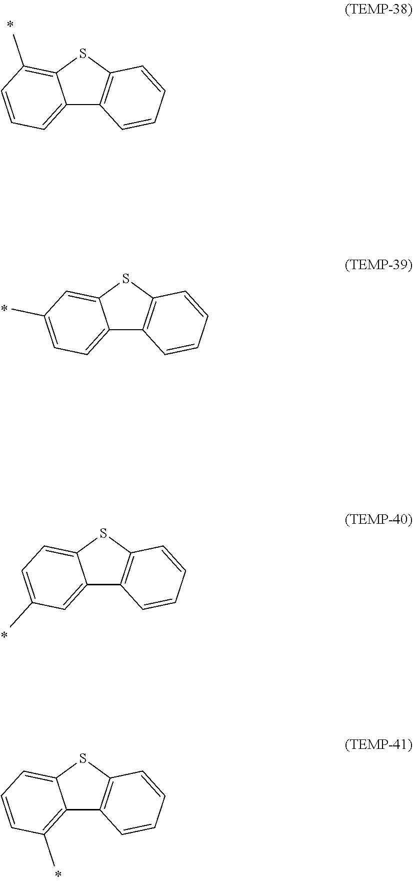

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

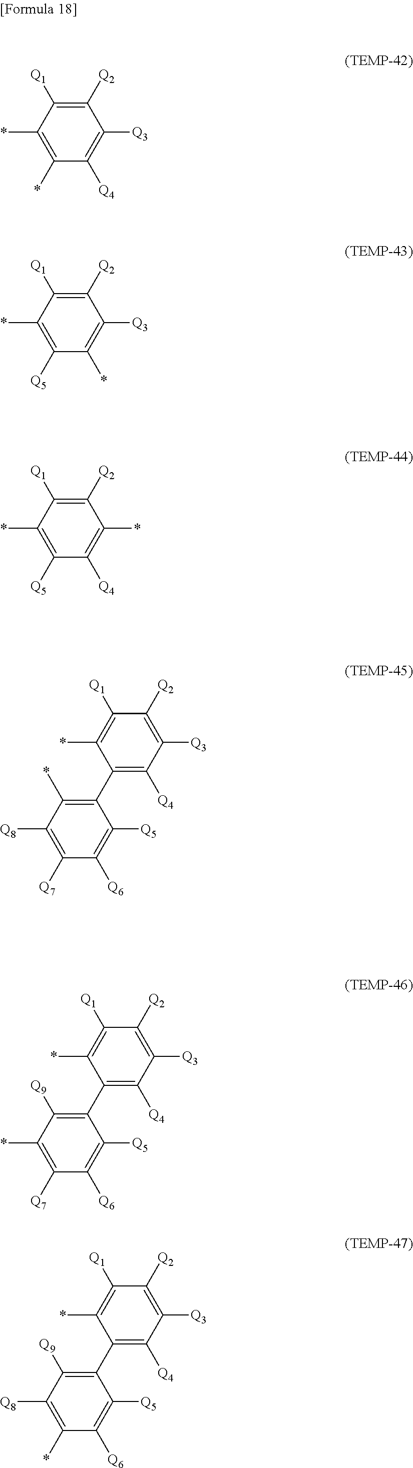

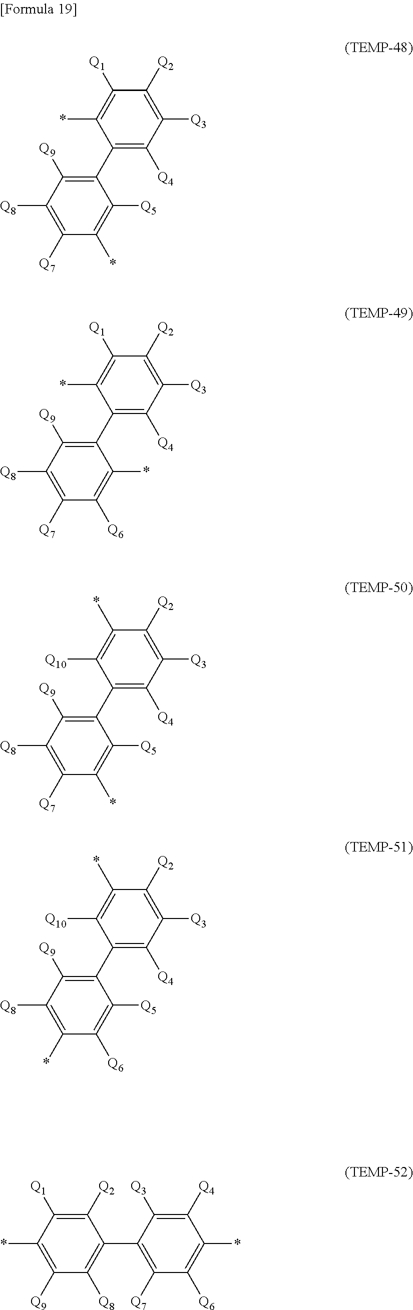

In the formulae (TEMP-42) to (TEMP-52), Q₁ to Q₁₀ each independently represent a hydrogen atom or a substituent.

In the formulae (TEMP-42) to (TEMP-52), * represents a bonding position.

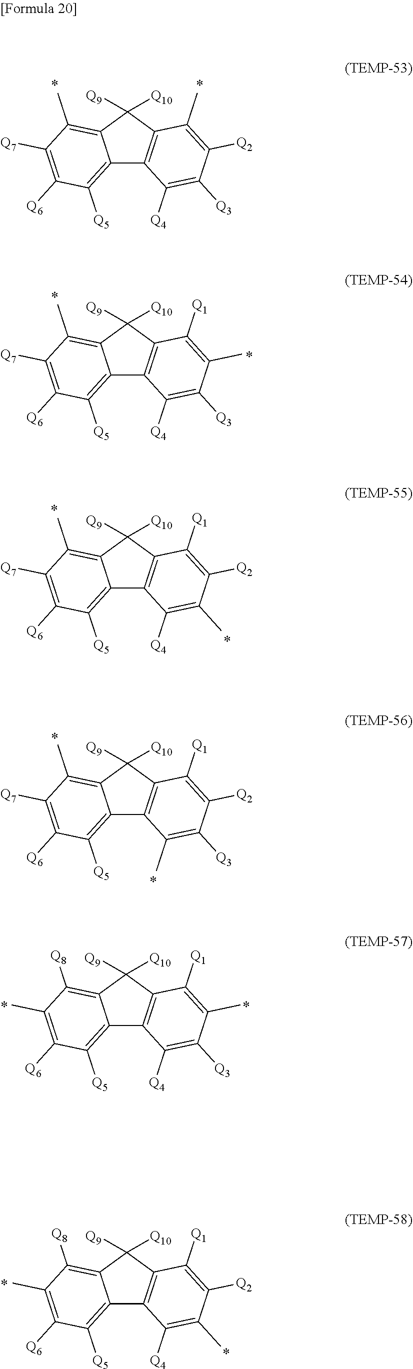

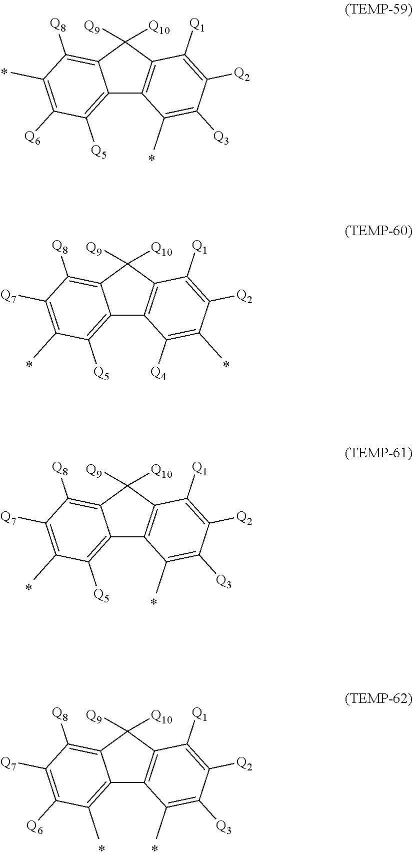

In the formulae (TEMP-53) to (TEMP-62), Q₁ to Q₁₀ each independently represent a hydrogen atom or a substituent.

In the formulae, Q₉ and Q₁₀ may be mutually bonded through a single bond to form a ring.

In the formulae (TEMP-53) to (TEMP-62), * represents a bonding position.

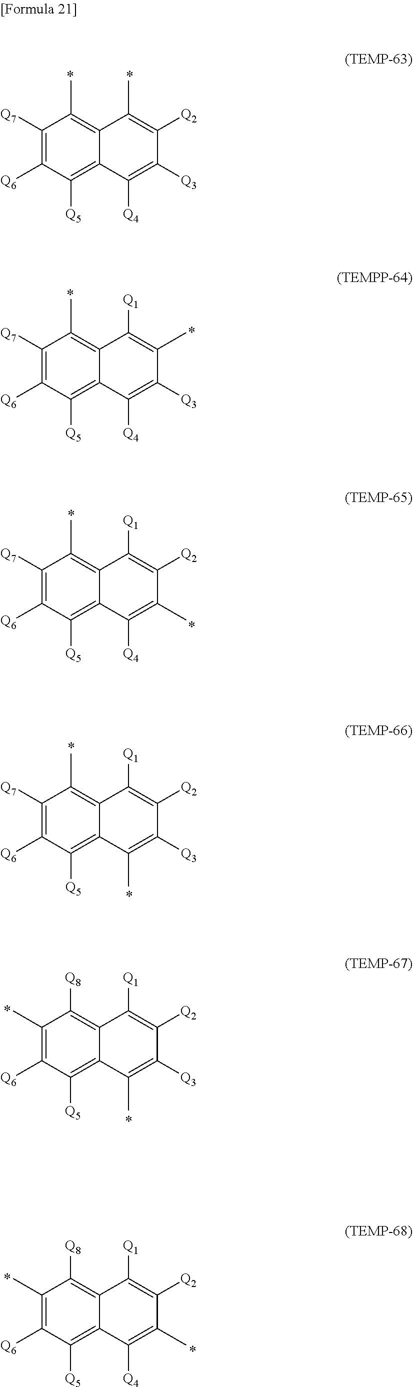

In the formulae (TEMP-63) to (TEMP-68), Q₁ to Q₈ each independently represent a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

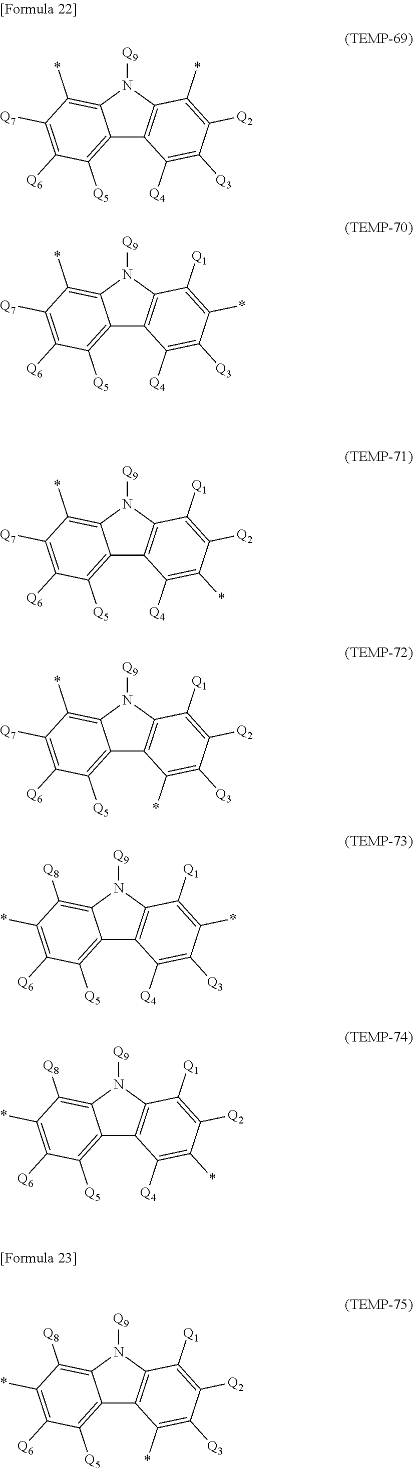

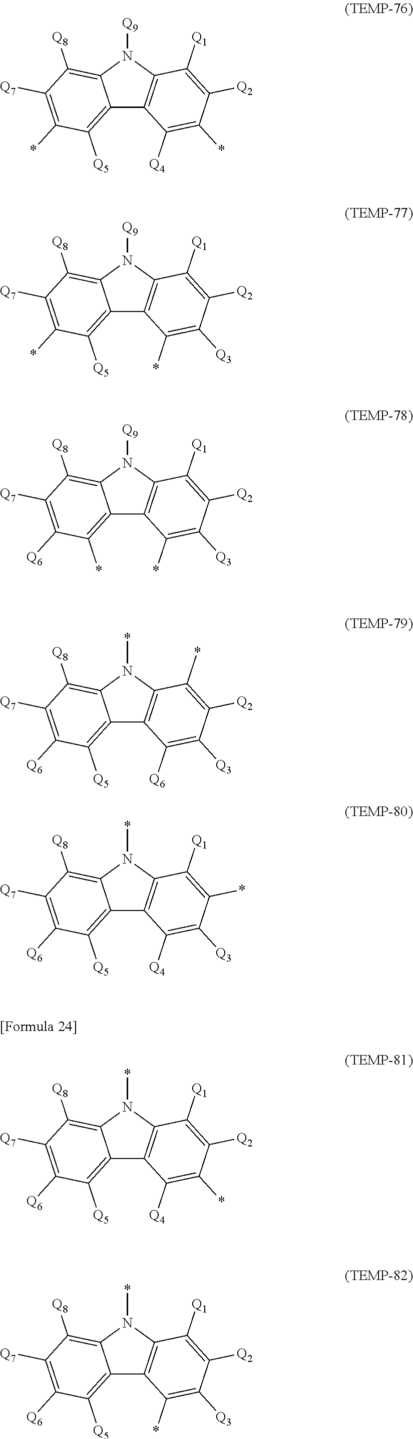

In the formulae (TEMP-69) to (TEMP-82), Q₁ to Q₉ each independently represent a hydrogen atom or a substituent.

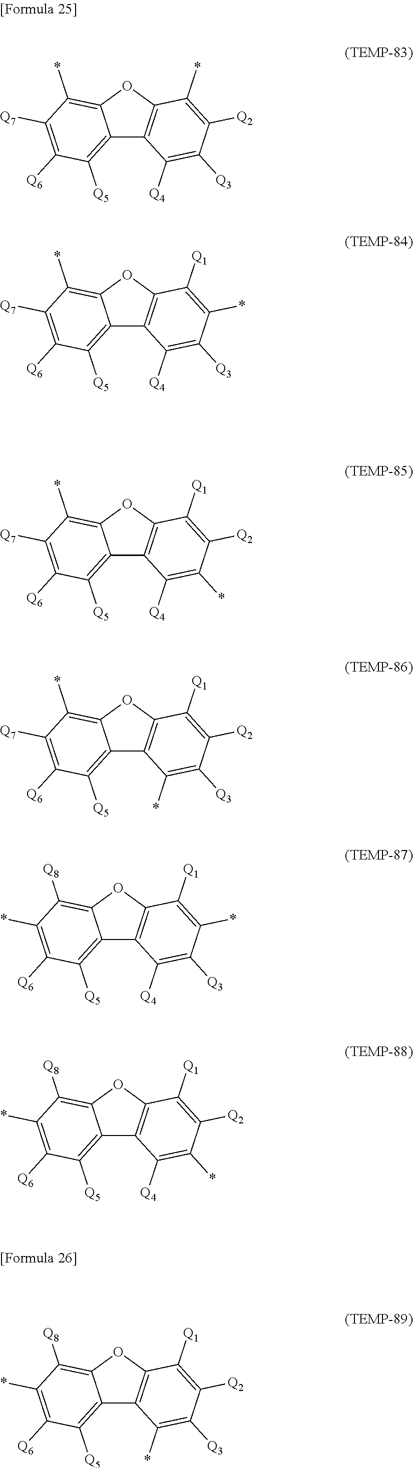

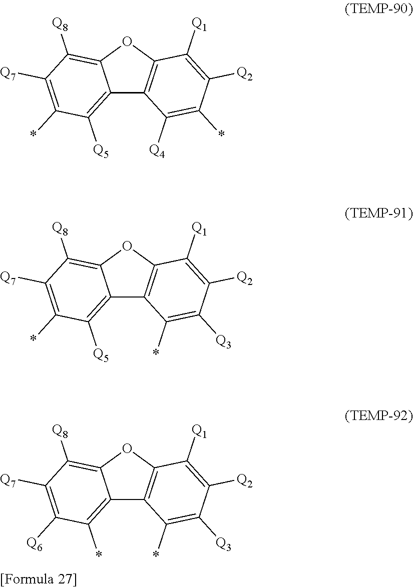

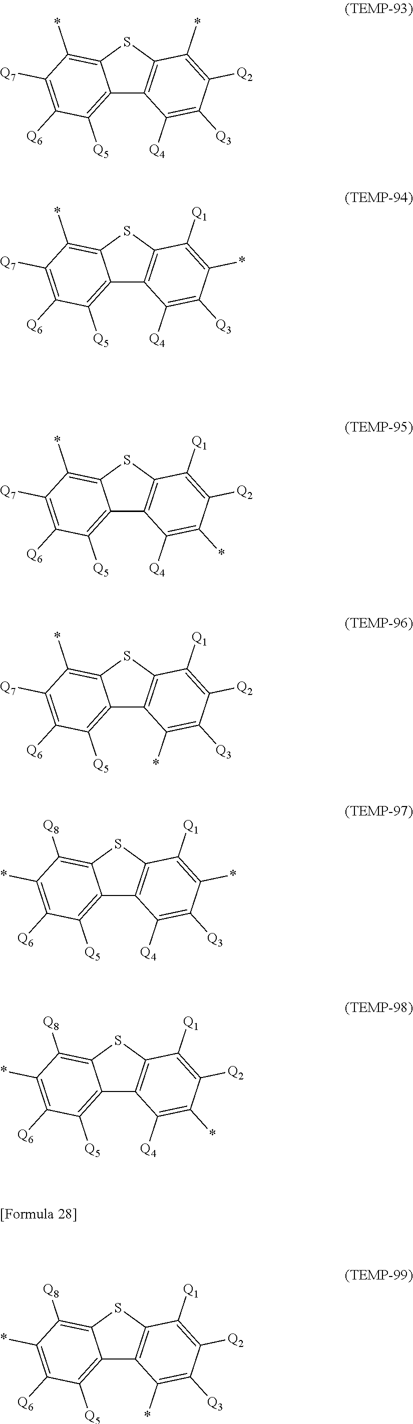

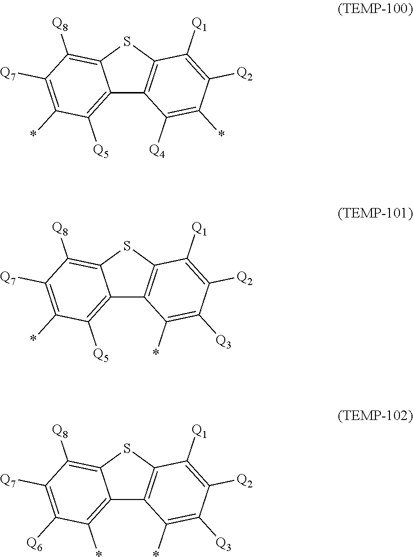

In the formulae (TEMP-83) to (TEMP-102), Q₁ to Q₈ are each independently a hydrogen atom or a substituent.

The above has described “Substituents in the present description”.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

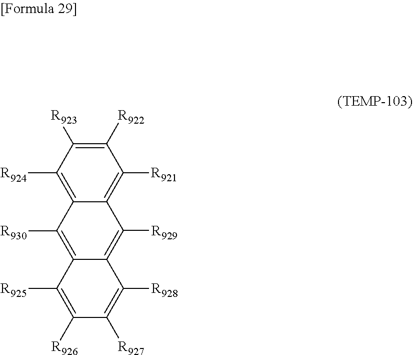

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

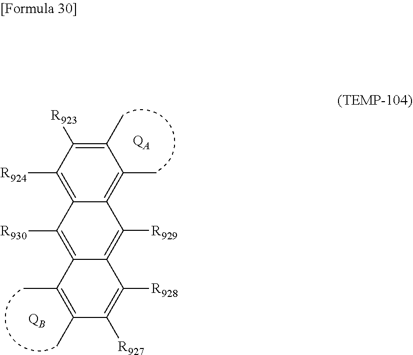

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

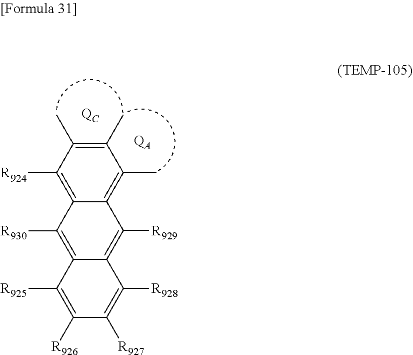

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

To “form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, a substituent for the substituted or unsubstituted group (sometimes referred to as an “optional substituent” hereinafter) is, for instance, a group selected from the group consisting of an unsubstituted alkyl group, having 1 to 50 carbon atoms, unsubstituted alkenyl group, having 2 to 50 carbon atoms, unsubstituted alkynyl group, having 2 to 50 carbon atoms, unsubstituted cycloalkyl group, having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, unsubstituted aryl group, having 6 to 50 ring carbon atoms, and unsubstituted heterocyclic group, having 5 to 50 ring atoms,
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, a substituent for the substituted or unsubstituted group is selected from the group consisting of alkyl group, having 1 to 50 carbon atoms, aryl group, having 6 to 50 ring carbon atoms, and heterocyclic group, having 5 to 50 ring atoms.

In an exemplary embodiment, a substituent for the “substituted or unsubstituted group” is selected from the group consisting of alkyl group, having 1 to 18 carbon atoms, aryl group, having 6 to 18 ring carbon atoms, and heterocyclic group, having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

First Compound

An organic EL device according to an exemplary embodiment of the invention has a first emitting layer containing a first compound. The first compound is a compound represented by the following formula (1A).

A compound according to a first exemplary embodiment (a compound represented by the formula (12X)) described later, a compound according to a second exemplary embodiment (a compound represented by the formula (120)), and a compound according to a third exemplary embodiment (a compound represented by the formula (1), (2), or (3)) are examples of a compound represented by the following formula (1A).

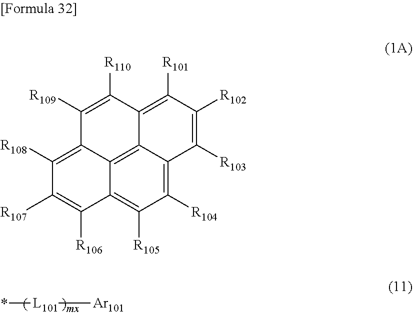

In the formula (1A):

R₁₀₁ to R₁₁₀ each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11),
at least one of R₁₀₁ to R₁₁₀ represents a group represented by the formula (11),
when a plurality of groups represented by the formula (11) are present, the plurality of groups represented by the formula (11) are mutually the same or different,
L₁₀₁ represents a single bond, a substituted or unsubstituted arylene group having 6 to 50 ring carbon atoms, or a substituted or unsubstituted divalent heterocyclic group having 5 to 50 ring atoms,
Ar₁₀₁ represents a substituted or unsubstituted aryl group having 6 to 50 ring carbon atoms, or a substituted or unsubstituted heterocyclic group having 5 to 50 ring atoms,
mx represents 0, 1, 2, 3, 4, or 5,
when two or more L₁₀₁s are present, the two or more L₁₀₁s are mutually the same or different,
when two or more Ar₁₀₁s are present, the two or more Ar₁₀₁s are mutually the same or different, and
* in the formula (11) represents the position of bonding to a pyrene ring in the formula (1A).

### First Exemplary Embodiment

The compound according to the first exemplary embodiment is a compound represented by the following formula (12X).

The compound according to the first exemplary embodiment (a compound represented by the following formula (12X)) is an example of the first compound (a compound that has at least one group represented by the formula (11) and is represented by the formula (1A)).

The compound according to the first exemplary embodiment is a compound in which R₁₀₁ in the formula (1A) of the first compound is bonded to the formula (11).

[Formula 33]

Py₁-L₁-L₂-Py₂ (12X)

In the formula (12X):
Py₁ and Py₂ each independently represent a substituted or unsubstituted 1-pyrenyl group;
L₁ and L₂ each independently represent a substituted or unsubstituted phenylene group, or a substituted or unsubstituted naphthylene group;
when L₁ and L₂ each independently represent a substituted or unsubstituted phenylene group, -L₁-L₂- in the formula (12X) represents a group represented by one of the following formulae (13-1) to (13-6), (10-1), (20-1), and (30-1), and when L₁ and L₂ each independently represent a substituted or unsubstituted naphthylene group, a bonding position of the naphthylene group as L₁ is different from a bonding position of the naphthylene group as L₂;
when Py₁ and Py₂ each independently represent a substituted 1-pyrenyl group, a substituent E of the substituted 1-pyrenyl group each independently represent a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms; and
when a plurality of substituents E are present, the plurality of substituents E are mutually the same or different.

A substituent F for “substituted or unsubstituted” in the substituent E is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

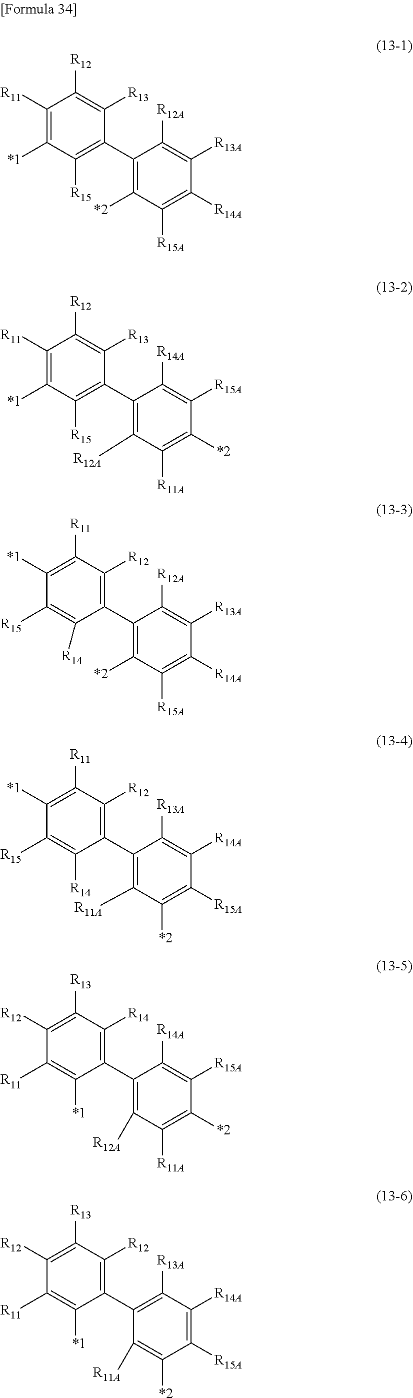

In the formulae (13-1) to (13-6), R₁₁ to R₁₅ and R_{11A} to R_{15A} each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms,
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms, a substituent for “substituted or unsubstituted” in R₁₁ to R₁₅ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group,
at least one combination of adjacent two or more of R₁₁ to R₁₅ are not mutually bonded to form no substituted or unsubstituted monocyclic ring or to form no substituted or unsubstituted fused ring,
at least one combination of adjacent two or more of R_{11A} to R_{15A} are not mutually bonded to form no substituted or unsubstituted monocyclic ring or to form no substituted or unsubstituted fused ring,
*1 in the formulae (13-1) to (13-6) represents the position of bonding to Py₁ in the formula (12X), and
*2 represents the position of bonding to Py₂ in the formula (12X).

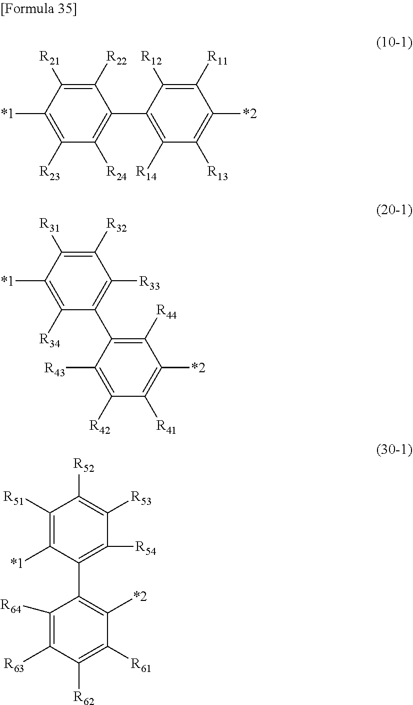

In the formula (10-1), at least one combination of a combination of R₁₁ and R₁₂, a combination of R₁₃ and R₁₄, a combination of R₂₁ and R₂₂, or a combination of R₂₃ and R₂₄ are mutually bonded to form a substituted or unsubstituted monocyclic ring, are mutually bonded to form a substituted or unsubstituted fused ring, or are not mutually bonded,
R₁₁, R₁₃, R₂₁, and R₂₃ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4),
R₁₂, R₁₄, R₂₂, and R₂₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4), and
at least one of R₁₁ to R₁₄ or R₂₁ to R₂₄ is not a hydrogen atom.

In the formulae (20-1) and (30-1):
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring, are mutually bonded to form a substituted or unsubstituted fused ring, or are not mutually bonded,
R₃₁ to R₃₄, R₄₁ to R₄₄, R₅₁ to R₅₄, and R₆₁ to R₆₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted spirobifluorenyl group, or a group represented by the following formula (4),
at least one of R₃₁ to R₃₄ or R₄₁ to R₄₄ is not a hydrogen atom, and
at least one of R₅₁ to R₅₄ or R₆₁ to R₆₄ is not a hydrogen atom,
in a compound represented by the formula (10-1), a combination of R₁₁ and R₁₃ and a combination of R₂₁ and R₂₃ are different combinations, or a combination of R₁₂ and R₁₄ and a combination of R₂₂ and R₂₄ are different combinations,
in a compound represented by the formula (20-1), R₃₁ is different from R₄₁, R₃₂ is different from R₄₂, R₃₃ is different from R₄₃, or R₃₄ is different from R₄₄,
in a compound represented by the formula (30-1), R₅₁ is different from R₆₁, R₅₂ is different from R₆₂, R₅₃ is different from R₆₃, or R₅₄ is different from R₆₄, and
*1 in the formulae (10-1), (20-1), and (30-1) represents the position of bonding to Py₁ in the formula (12X), and *2 represents the position of bonding to Py₂ in the formula (12X).

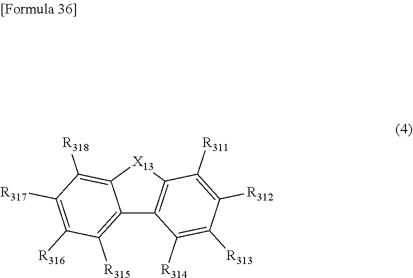

In the formula (4),
X₁₃ represents an oxygen atom, a sulfur atom, or NR₃₁₉,
at least one combination of adjacent two or more of R₃₁₁ to R₃₁₈ are mutually bonded to form a substituted or unsubstituted monocyclic ring, are mutually bonded to form a substituted or unsubstituted fused ring, or are not mutually bonded,
R₃₁₁ to R₃₁₉ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group, and
any one of R₃₁₁ to R₃₁₉ that do not form the substituted or unsubstituted monocyclic ring and do not form the substituted or unsubstituted fused ring is a single bond bonded to at least one of R₁₁ to R₁₄, R₂₁ to R₂₄, R₃₁ to R₃₄, R₄₁ to R₄₄, R₅₁ to R₅₄, Or R₆₁ to R₆₄.

In the formulae (10-1), (20-1), and (30-1) and the formula (4), a substituent for “substituted or unsubstituted” in R₁₁ to R₁₄, R₂₁ to R₂₄, R₃₁ to R₃₄, R₄₁ to R₄₄, R₅₁ to R₅₄, R₆₁ to R₆₄, and R₃₁₁ to R₃₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

A bispyrene bonded via one linker (a linking group) is known as a host material contained in the emitting layer. However, a bispyrene bonded via one linker tends to have a twisted molecular structure, tends to reduce the planarity of the whole compound, and may reduce hole transportability.

In contrast, a compound represented by the formula (12X) according to the first exemplary embodiment (hereinafter referred to as “the compound according to the first exemplary embodiment”) has a structure in which two 1-pyrenyl groups (Py₁ and Py₂) are bonded via -L₁-L₂- (two linkers composed of L₁ serving as a linking group and L₂ serving as a linking group), and -L₁-L₂- has an asymmetric structure. The compound according to the first exemplary embodiment with such a structure is less likely to have a twisted molecular structure, easily maintains the planarity of the whole compound, and improves hole transportability.

Thus, the compound according to the first exemplary embodiment contained in the emitting layer can improve luminous efficiency.

The compound according to the first exemplary embodiment has an asymmetric structure as a whole when -L₁-L₂- in the formula (12X) has an asymmetric structure.

In the first exemplary embodiment, that -L₁-L₂- in the formula (12X) has an asymmetric structure means that -L₁-L₂- in the formula (12X) has one of the following structures (asymmetric structures 1 to 4).

Asymmetric structure 1: L₁ and L₂ each independently represent a substituted or unsubstituted phenylene group, and the bonding position of the phenylene group in L₁ is different from the bonding position of the phenylene group in L₂.

Asymmetric structure 2: L₁ and L₂ each independently represent a substituted or unsubstituted naphthylene group, and the bonding position of the naphthylene group in L₁ is different from the bonding position of the naphthylene group in L₂.

Asymmetric structure 3: One of L₁ and L₂ represents a substituted or unsubstituted phenylene group, and the other of L₁ and L₂ represents a substituted or unsubstituted naphthylene group.

Asymmetric structure 4: Although L₁ and L₂ each independently represent a substituted or unsubstituted phenylene group, and the bonding position of the phenylene group in L₁ is the same as the bonding position of the phenylene group in L₂, the structure of L₁ and the structure of L₂ are mutually different including substituents.

First, the asymmetric structures 1 to 3 are described with respect to compounds represented by the following formulae (130X), (130Y), and (130Z) in which carbon positions are numbered.

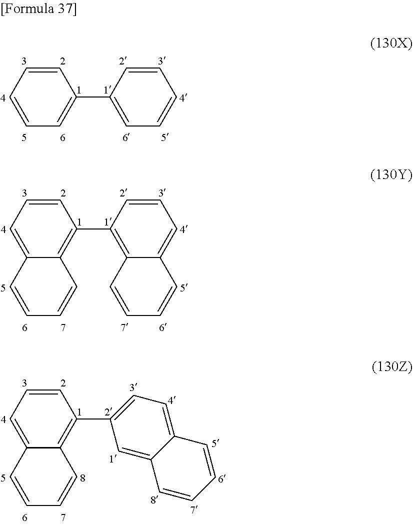

Asymmetric Structure 1

Examples of -L₁-L₂- with the asymmetric structure 1 include groups represented by the formulae (13-1) to (13-6).

The asymmetric structure 1 is described with respect to the following formulae (130-1) to (130-3), which are examples of the groups represented by the formulae (13-1) to (13-3).

For example, a group represented by the following formula (130-1) has an asymmetric structure because the bonding position of *1 (No. 5 in the formula (130X)) is different from the bonding position of *2 (No. 6′ in the formula (130X)).

A group represented by the following formula (130-2) has an asymmetric structure because the bonding position of *1 (No. 5 in the formula (130X)) is different from the bonding position of *2 (No. 4′ in the formula (130X)).

A structure represented by the following formula (130-3) has an asymmetric structure because the bonding position of *1 (No. 4 in the formula (130X)) is different from the bonding position of *2 (No. 6′ in the formula (130X)).

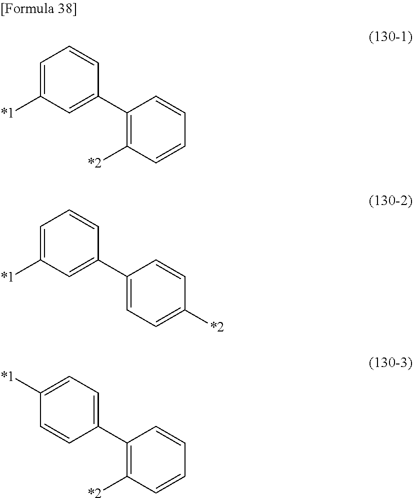

Asymmetric Structure 2

Examples of -L₁-L₂- with the asymmetric structure 2 include groups represented by the formulae (13-49) to (13-69) described later in the second exemplary embodiment. The asymmetric structure 2 is described with respect to the following formulae (130-52) and (130-53), which are examples of the groups represented by the formulae (13-52) and (13-53) described later in the second exemplary embodiment.

For example, a group represented by the following formula (130-52) has an asymmetric structure because the bonding position of *1 (No. 4 in the formula (130Y)) is different from the bonding position of *2 (No. 5′ in the formula (130Y)), though the bonding position (No. 1 in the formula (130Y)) of L₁ (a naphthylene group) bonded to L₂ is the same as the bonding position (No. 1′ in the formula (130Y)) of L₂ (a naphthylene group) bonded to L₁.

A group represented by the following formula (130-53) has an asymmetric structure because at least the bonding position (No. 1 in the formula (130Z)) of L₁ (a naphthylene group) bonded to L₂ is different from the bonding position (No. 2′ in the formula (130Z)) of L₂ (a naphthylene group) bonded to L₁.

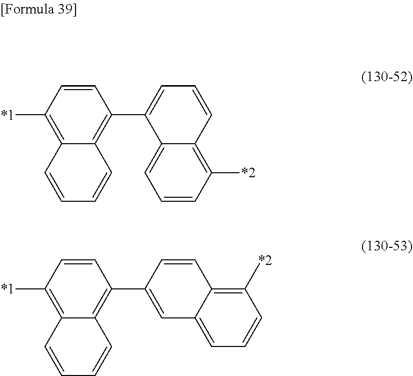

Asymmetric Structure 3

Examples of -L₁-L₂- with the asymmetric structure 3 include groups represented by the formulae (13-7) to (13-48) described later in the second exemplary embodiment. The groups represented by the formulae (13-7) to (13-48) have an asymmetric structure because the structure of L₁ is different from the structure of L₂.

The asymmetric structure 4 is described below.

Asymmetric Structure 4

Examples of -L₁-L₂- with the asymmetric structure 4 include groups represented by one of the formulae (10-1), (20-1), and (30-1).

The asymmetric structure 4 is described with respect to the following formulae (100-1), (200-1), and (300-1), which are embodiments of the groups represented by the formulae (10-1), (20-1), and (30-1).

For example, a group represented by the following formula (100-1) has an asymmetric structure because the phenylene group of L₁ having a substituent (a naphthylene group) is different from the unsubstituted phenylene group of L₂, though the bonding position of *1 (No. 4 in the formula (130X)) is the same as the bonding position of *2 (No. 4′ in the formula (130X)).

A group represented by the following formula (200-1) has an asymmetric structure because the phenylene group of L₂ having a substituent (a phenylene group) is different from the unsubstituted phenylene group of L₁, though the bonding position of *1 (No. 5 in the formula (130X)) is the same as the bonding position of *2 (No. 3′ in the formula (130X)).

A group represented by the following formula (300-1) has an asymmetric structure because the bonding position of a phenylene group bonded to the phenylene group of L₁ (No. 4 in the formula (130X)) is different from the bonding position of a phenylene group bonded to the phenylene group of L₂ (No. 3 in the formula (130X)), though the bonding position of *1 (No. 6 in the formula (130X)) is the same as the bonding position of *2 (No. 6′ in the formula (130X)), and both the phenylene group of L₁ and the phenylene group of L₂ have the same substituent (the phenylene group).

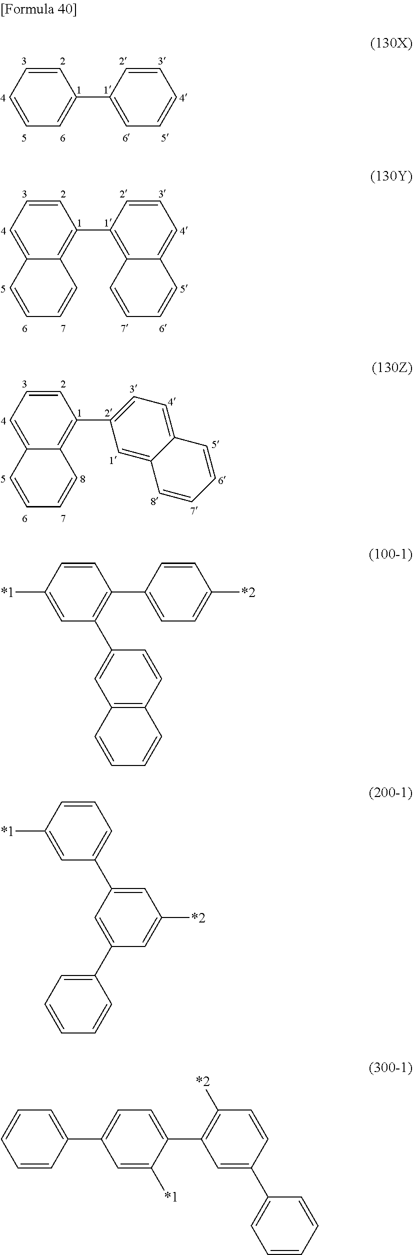

In the compound according to the first exemplary embodiment, a compound represented by the formula (12X) is preferably a compound represented by the following formula (120).

In the compound according to the first exemplary embodiment, a compound represented by the following formula (120) represents the same as the compound according to the second exemplary embodiment.

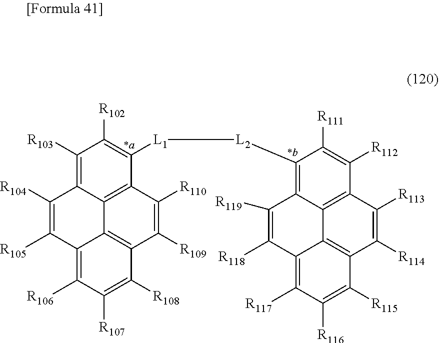

In the formula (120), a substituted or unsubstituted 1-pyrenyl group with R₁₀₂ to R₁₁₀, a substituted or unsubstituted 1-pyrenyl group with R₁₁₁ to R₁₁₉, and L₁ and L₂ represent the same as Py₁, Py₂, L₁, and L₂, respectively, in the formula (12X).

In the formula (120), L₁, L₂, and R₁₀₂ to R₁₁₉ represent the same as L₁, L₂, and R₁₀₂ to R₁₁₉, respectively, in the compound according to the second exemplary embodiment, and the same applies to preferred ranges.

In the formula (120), R₁₁ to R₁₅, R₂₁ to R₂₇, and R₃₁ to R₃₇ in the formulae (11) to (13) representing L₁ represent the same as R₁₁ to R₁₅, R₂₁ to R₂₇, and R₃₁ to R₃₇, respectively, in the compound according to the second exemplary embodiment, and the same applies to preferred ranges.

In the formula (120), R_{11A} to R_{15A}, R_{21A} to R_{27A}, and R_{31A} to R_{37A} in the formulae (11A) to (13A) representing L₂ represent the same as R_{11A} to R_{15A}, R_{21A} to R_{27A}, and R_{31A} to R_{37A}, respectively, in the compound according to the second exemplary embodiment, and the same applies to preferred ranges.

In the compound according to the first exemplary embodiment, a compound represented by the formula (12X) is preferably a compound represented by the following formula (1), (2), or (3).

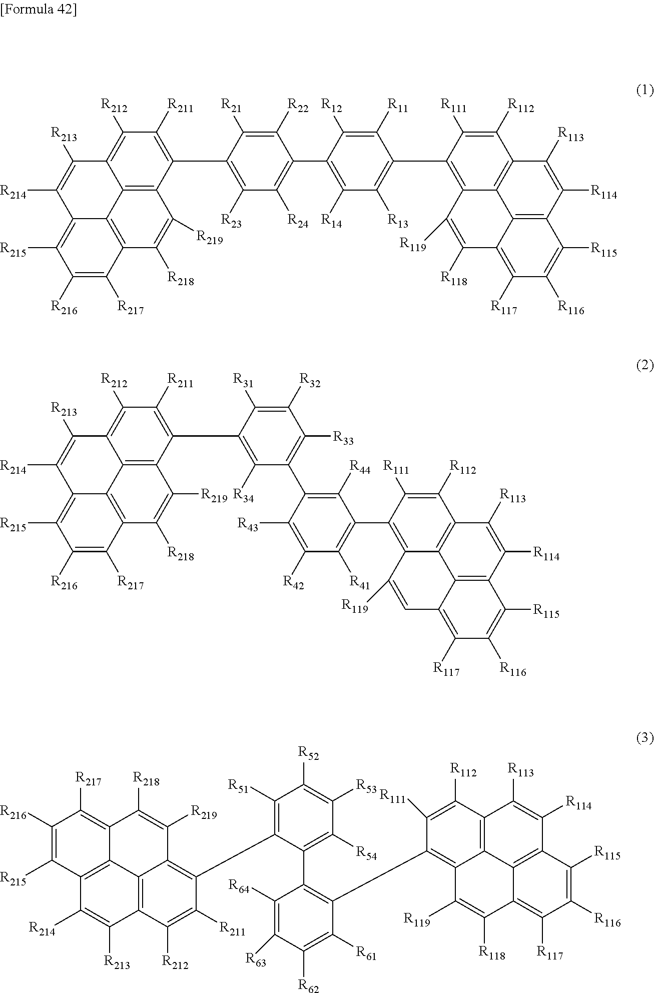

In the formulae (1) to (3), a substituted or unsubstituted 1-pyrenyl group with R₂₁₁ to R₂₁₉ represents the same as the substituent E of the substituted 1-pyrenyl group in the formula (12X), and a substituted or unsubstituted 1-pyrenyl group with R₁₁₁ to R₁₁₉ represents the same as the substituent E of the substituted 1-pyrenyl group in the formula (12X),
a substituted or unsubstituted biphenylene group with R₁₁ to R₁₄ and R₂₁ to R₂₄ in the formula (1), a substituted or unsubstituted biphenylene group with R₃₁ to R₃₄ and R₄₁ to R₄₄ in the formula (2), and a substituted or unsubstituted biphenylene group with R₅₁ to R₅₄ and R₆₁ to R₆₄ in the formula (3) each independently represent the same as -L₁-L₂- in the formula (12X).

In the formulae (1) to (3), R₁₁₁ to R₁₁₉, R₂₁₁ to R₂₁₉, R₁₁ to R₁₄, R₂₁ to R₂₄, R₃₁ to R₃₄, R₄₁ to R₄₄, R₅₁ to R₅₄, and R₆₁ to R₆₄ represent the same as R₁₁₁ to R₁₁₉, R₂₁₁ to R₂₁₉, R₁₁ to R₁₄, R₂₁ to R₂₄, R₃₁ to R₃₄, R₄₁ to R₄₄, R₅₁ to R₅₄, and R₆₁ to R₆₄, respectively, in the compound according to the third exemplary embodiment, and the same applies to preferred ranges.

In the formula (2), preferably, R₃₁ is different from R₄₁, R₃₂ is different from R₄₂, R₃₃ is different from R₄₃, or R₃₄ is different from R₄₄. In the formula (3), R₅₁ is different from R₆₁, R₅₂ is different from R₆₂, R₅₃ is different from R₆₃, or R₅₄ is different from R₆₄.

### Method for Producing Compound According to First Exemplary Embodiment

The compound according to the first exemplary embodiment (a compound represented by the formula (12X)) can be produced by a known method. The compound according to the first exemplary embodiment can also be produced in accordance with a known method by using a known alternative reaction and raw materials suitable for the target compound.

Specific examples of the compound according to the first exemplary embodiment (a compound represented by the formula (12X)) are described in the specific examples of the compound according to the second exemplary embodiment (a compound represented by the formula (120)) or in the specific examples of the compound according to the third exemplary embodiment (a compound represented by the formula (1), (2), or (3)).

### Second Exemplary Embodiment

### Compound

The compound according to the second exemplary embodiment is a compound represented by the following formula (120).

A compound represented by the following formula (120) is one example of the first compound (a compound represented by the formula (1A)).

A compound represented by the following formula (120) is a compound in which R₁₀₁ in the formula (1A) is bonded to the formula (11) via a single bond.

A compound represented by the following formula (120) is one example of the compound according to the first exemplary embodiment (a compound represented by the formula (12X)).

The compound according to the second exemplary embodiment has an asymmetric structure as a whole because -L₁-L₂- in the following formula (120) has an asymmetric structure (one of the asymmetric structures 1 to 3).

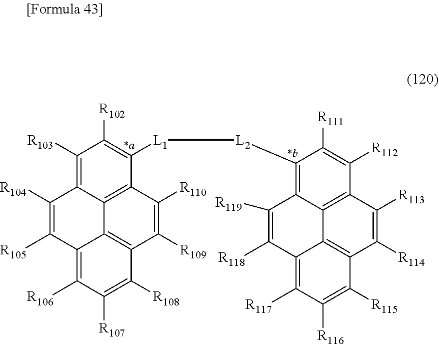

In the formula (120):
L₁ represents a group represented by one of the following formulae (11) to (13), and L₂ represents a group represented by one of the following formulae (11A) to (13A),
R₁₀₂ to R₁₁₉ each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms,
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms, and
a substituent for “substituted or unsubstituted” in R₁₀₂ to R₁₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

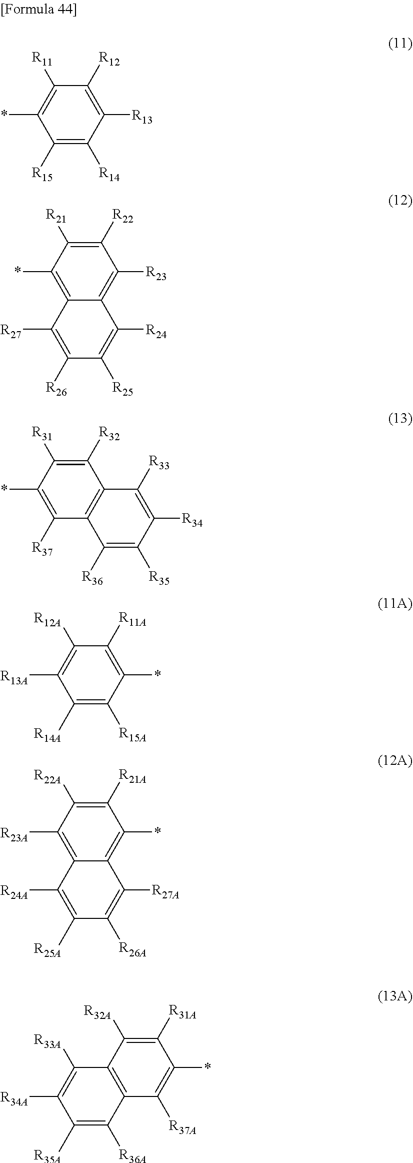

In the formulae (11) to (13) and (11A) to (13A):
R₁₁ to R₁₅, R₂₁ to R₂₇, R₃₁ to R₃₇, R_{11A} to R_{15A}, R_{21A} to R_{27A}, and R_{31A} to R_{37A} each independently represent a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms,
a substituent for “substituted or unsubstituted” in R₁₁ to R₁₅, R₂₁ to R₂₇, and R₃₁ to R₃₇ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group,
at least one combination of adjacent two or more of R₁₁ to R₁₅ are not mutually bonded to form no substituted or unsubstituted monocyclic ring or to form no substituted or unsubstituted fused ring,
at least one combination of adjacent two or more of R_{11A} to R_{15A} are not mutually bonded to form no substituted or unsubstituted monocyclic ring or to form no substituted or unsubstituted fused ring,
at least one combination of adjacent two or more of R₂₁ to R₂₇ are not mutually bonded to form no substituted or unsubstituted monocyclic ring or to form no substituted or unsubstituted fused ring,
at least one combination of adjacent two or more of R_{21A} to R_{27A} are not mutually bonded to form no substituted or unsubstituted monocyclic ring or to form no substituted or unsubstituted fused ring,
at least one combination of adjacent two or more of R₃₁ to R₃₇ are not mutually bonded to form no substituted or unsubstituted monocyclic ring or to form no substituted or unsubstituted fused ring,
at least one combination of adjacent two or more of R_{31A} to R_{37A} are not mutually bonded to form no substituted or unsubstituted monocyclic ring or to form no substituted or unsubstituted fused ring,
in L₁ represented by the formulae (11) to (13), * represents the position of bonding to *a in the formula (120), and any one of R₁₁ to R₁₅, R₂₁ to R₂₇, and R₃₁ to R₃₇ is a single bond bonded to L₂,
in L₂ represented by the formulae (11A) to (13A), * represents the position of bonding to *b in the formula (120), and any one of R_{11A} to R_{15A}, R_{21A} to R_{27A}, and R_{31A} to R_{37A} is a single bond bonded to L₁,
when R₁₂ or R₁₄ in L₁ is a single bond bonded to L₂, one of R_{11A}, R_{13A}, R_{15A}, R_{21A} to R_{27A}, and R_{31A} to R_{37A} in L₂ is a single bond bonded to L₁,
when R₁₁ or R₁₅ in L₁ is a single bond bonded to L₂, one of R_{12A}, R_{13A}, R_{14A}, R_{21A} to R_{27A}, and R_{31A} to R_{37A} in L₂ is a single bond bonded to L₁,
when R₁₃ in L₁ is a single bond bonded to L₂, one of R_{11A}, R_{12A}, R_{14A}, R_{15A}, R_{21A} to R_{27A}, and R_{31A} to R_{37A} in L₂ is a single bond bonded to L₁,
when R₂₁ in L₁ is a single bond bonded to L₂, one of R_{11A} to R_{15A}, R_{22A} to R_{27A}, and R_{31A} to R_{37A} in L₂ is a single bond bonded to L₁,
when R₂₂ in L₁ is a single bond bonded to L₂, one of R_{11A} to R_{15A}, R_{21A}, R_{23A} to R_{27A}, and R_{31A} to R_{37A} in L₂ is a single bond bonded to L₁,
when R₂₃ in L₁ is a single bond bonded to L₂, one of R_{11A} to R_{15A}, R_{21A} to R_{22A}, R_{24A} to R_{27A}, and R_{31A} to R_{37A} in L₂ is a single bond bonded to L₁,
when R₂₄ in L₁ is a single bond bonded to L₂, one of R_{11A} to R_{15A}, R_{21A} to R_{23A}, R_{25A} to R_{27A}, and R_{31A} to R_{37A} in L₂ is a single bond bonded to L₁,
when R₂₅ in L₁ is a single bond bonded to L₂, one of R_{11A} to R_{15A}, R_{21A} to R_{24A}, R_{26A} to R_{27A}, and R_{31A} to R_{37A} in L₂ is a single bond bonded to L₁,
when R₂₆ in L₁ is a single bond bonded to L₂, one of R_{11A} to R_{15A}, R_{21A} to R_{25A}, R_{27A}, and R_{31A} to R_{37A} in L₂ is a single bond bonded to L₁,
when R₂₇ in L₁ is a single bond bonded to L₂, one of R_{11A} to R_{15A}, R_{21A} to R_{26A}, and R_{31A} to R_{37A} in L₂ is a single bond bonded to L₁,
when R₃₁ in L₁ is a single bond bonded to L₂, one of R_{11A} to R_{15A}, R_{21A} to R_{27A}, and R_{32A} to R_{37A} in L₂ is a single bond bonded to L₁,
when R₃₂ in L₁ is a single bond bonded to L₂, one of R_{11A} to R_{15A}, R_{21A} to R_{27A}, R_{31A}, and R_{33A} to R_{37A} in L₂ is a single bond bonded to L₁,
when R₃₃ in L₁ is a single bond bonded to L₂, one of R_{11A} to R_{15A}, R_{21A} to R_{27A}, R_{31A} to R_{32A}, and R_{34A} to R_{37A} in L₂ is a single bond bonded to L₁,
when R₃₄ in L₁ is a single bond bonded to L₂, one of R_{11A} to R_{15A}, R_{21A} to R_{27A}, R_{31A} to R_{33A}, and R_{35A} to R_{37A} in L₂ is a single bond bonded to L₁,
when R₃₅ in L₁ is a single bond bonded to L₂, one of R_{11A} to R_{15A}, R_{21A} to R_{27A}, R_{31A} to R_{34A}, and R_{36A} to R_{37A} in L₂ is a single bond bonded to L₁,
when R₃₆ in L₁ is a single bond bonded to L₂, one of R_{11A} to R_{15A}, R_{21A} to R_{27A}, R_{31A} to R_{35A}, and R_{37A} in L₂ is a single bond bonded to L₁, and
when R₃₇ in L₁ is a single bond bonded to L₂, one of R_{11A} to R_{15A}, R_{21A} to R_{27A}, and R_{31A} to R_{36A} in L₂ is a single bond bonded to L₁.

In the compound according to the second exemplary embodiment, -L₁-L₂- preferably represents a group represented by one of the following formulae (13-1) to (13-69).

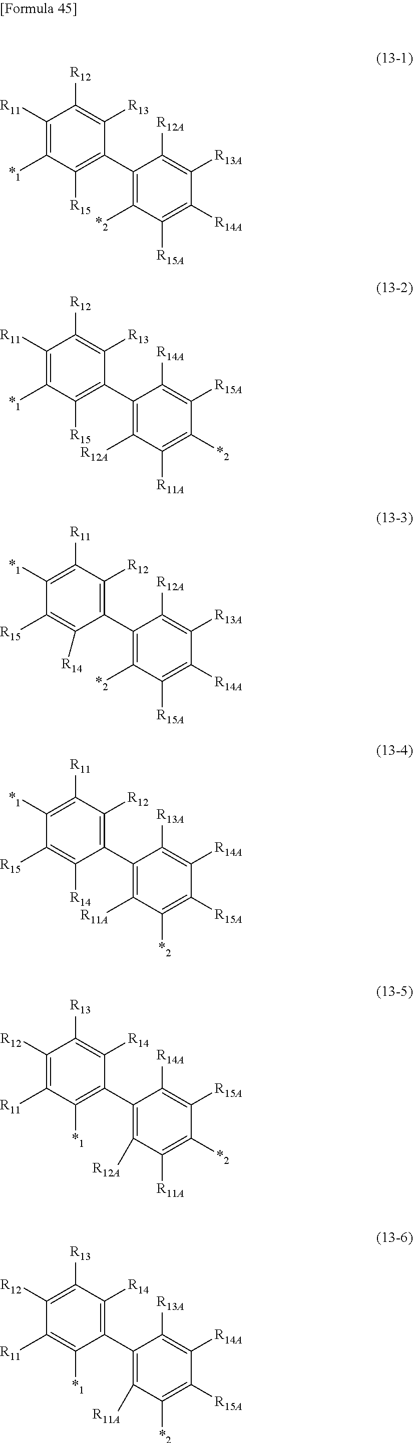

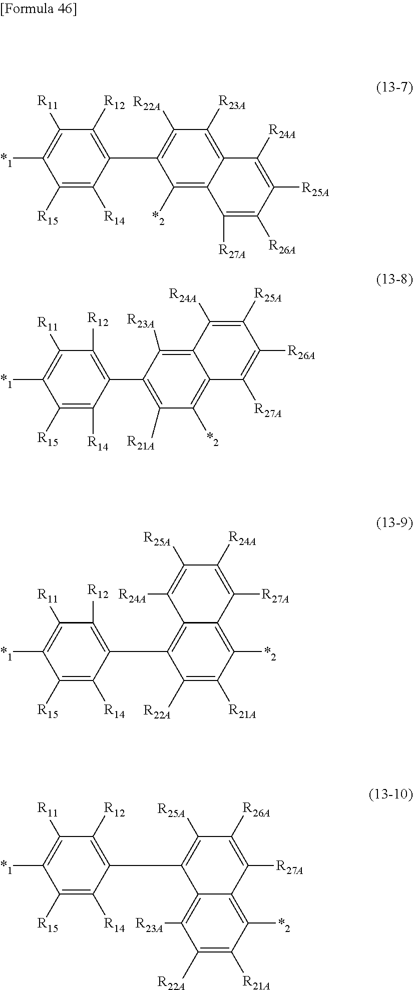

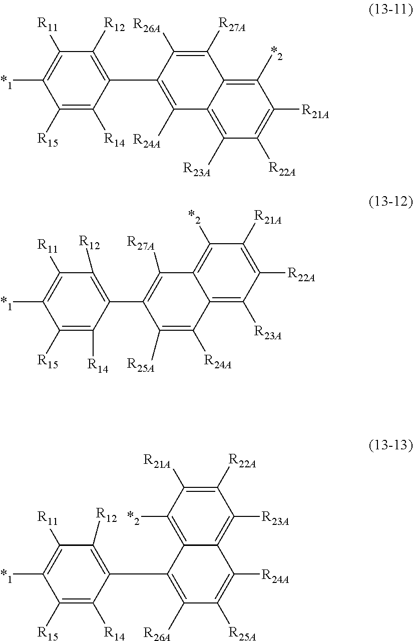

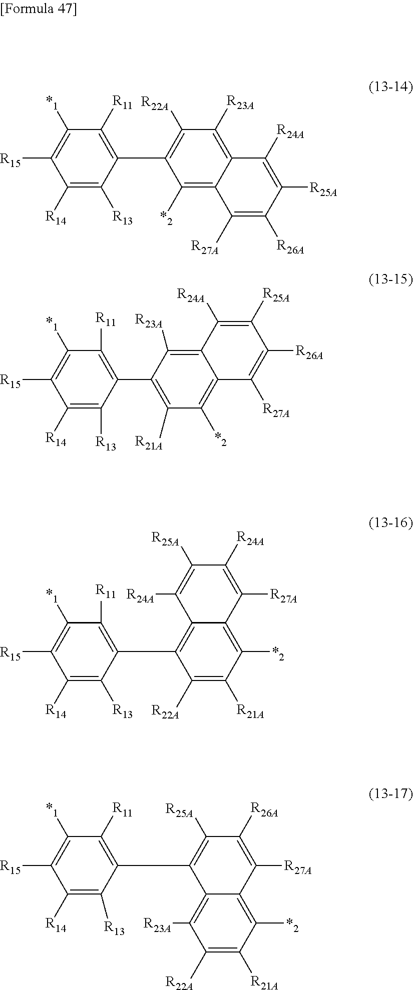

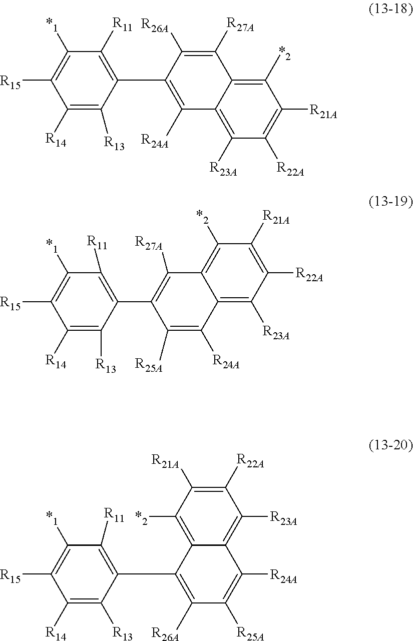

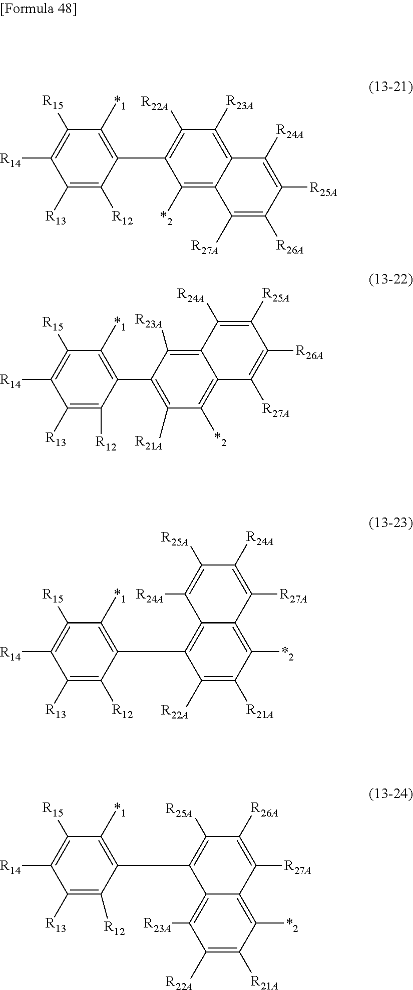

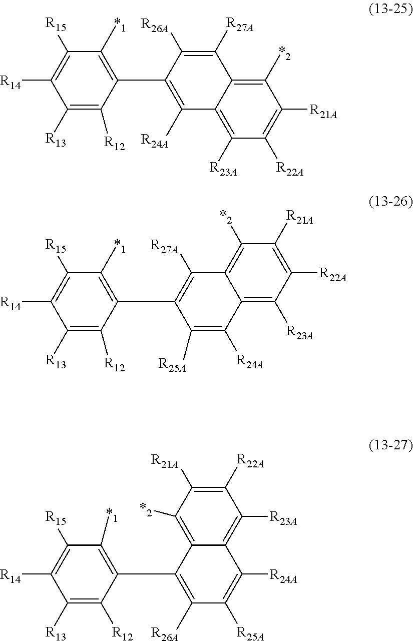

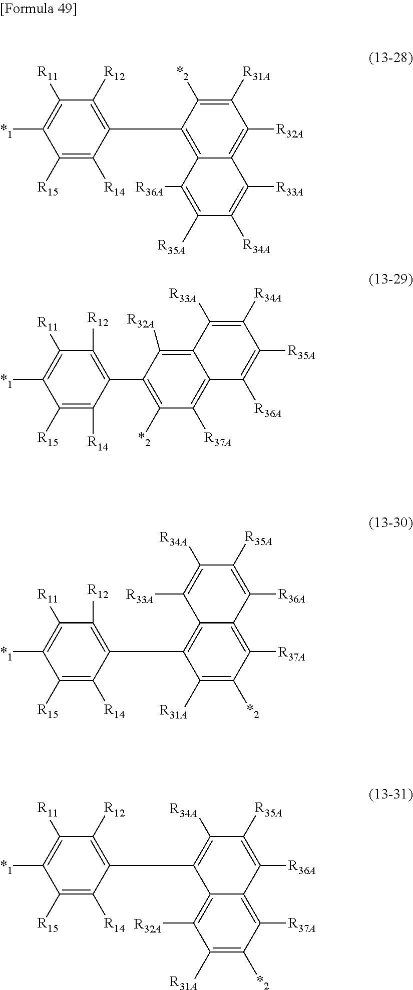

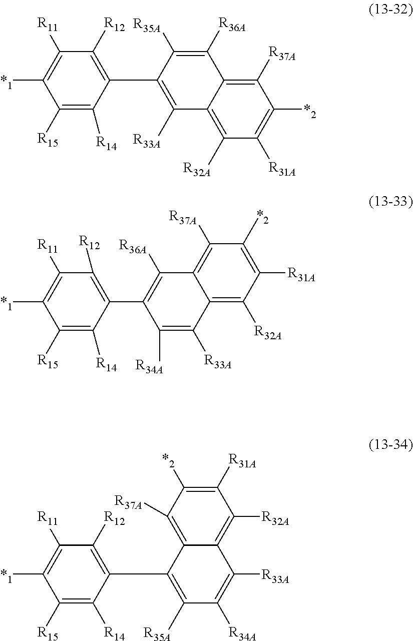

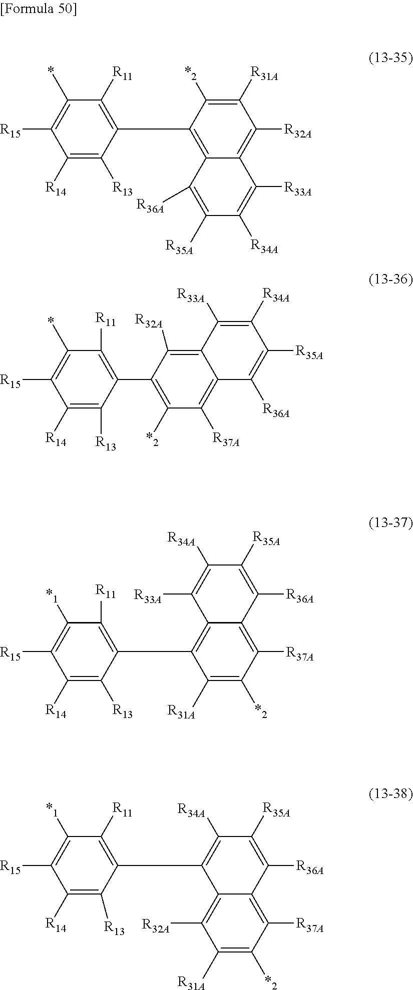

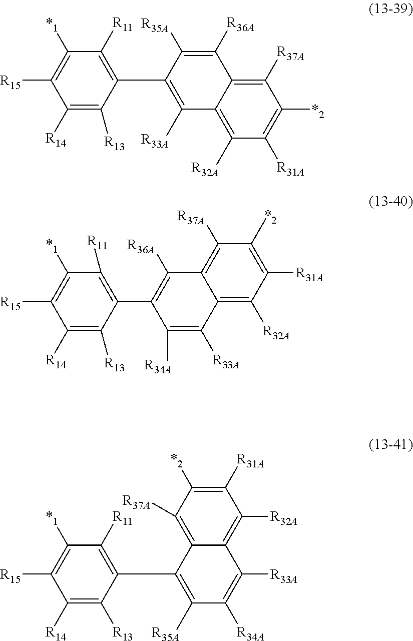

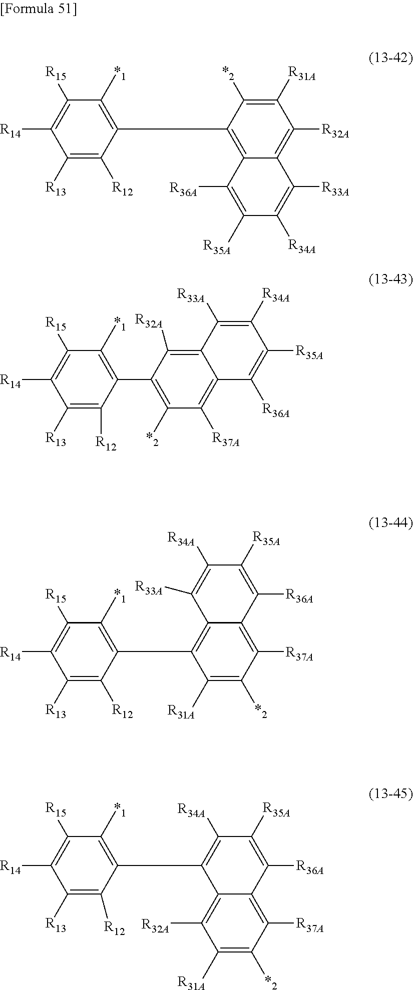

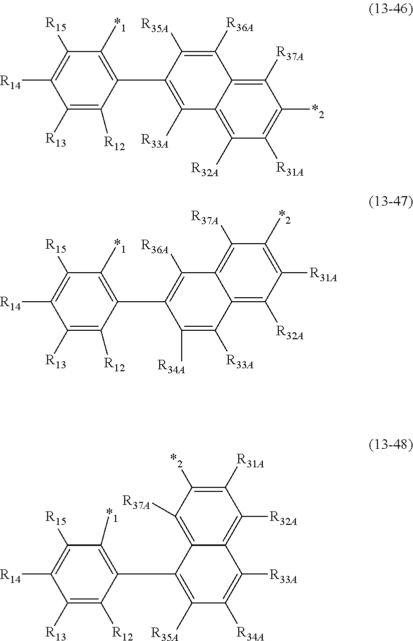

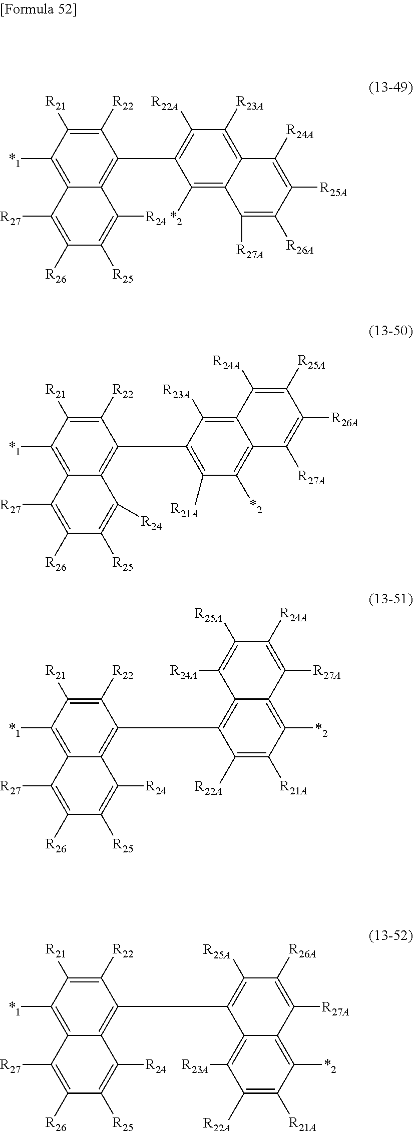

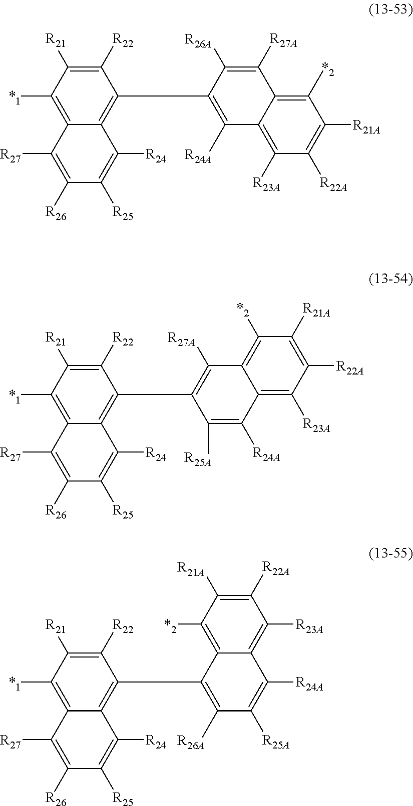

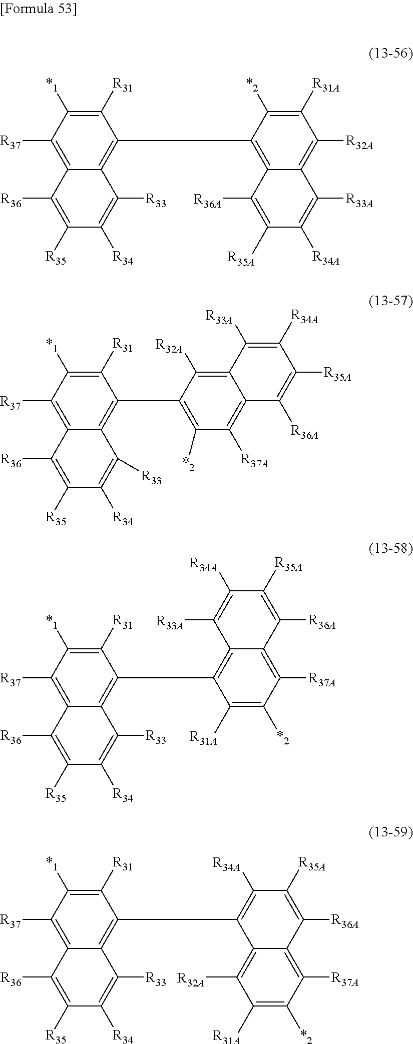

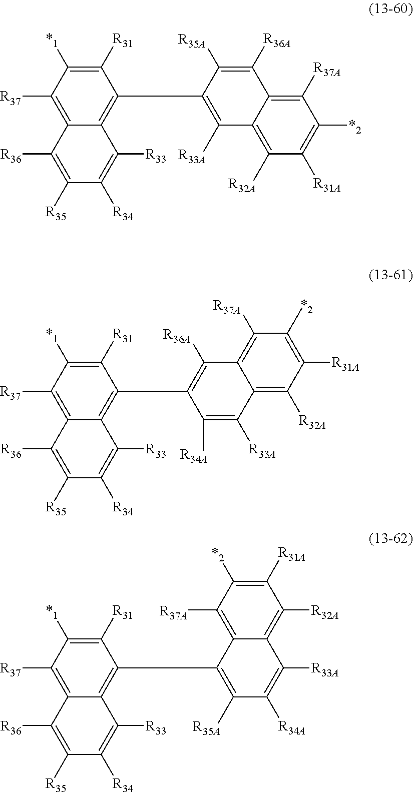

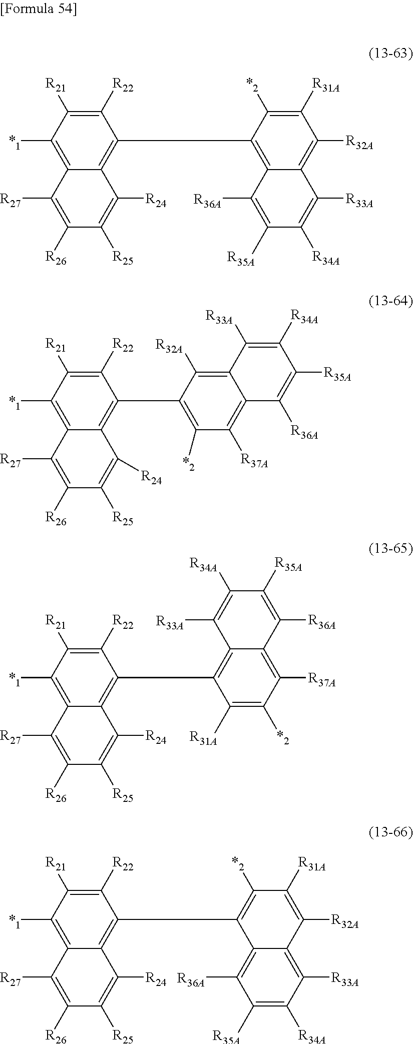

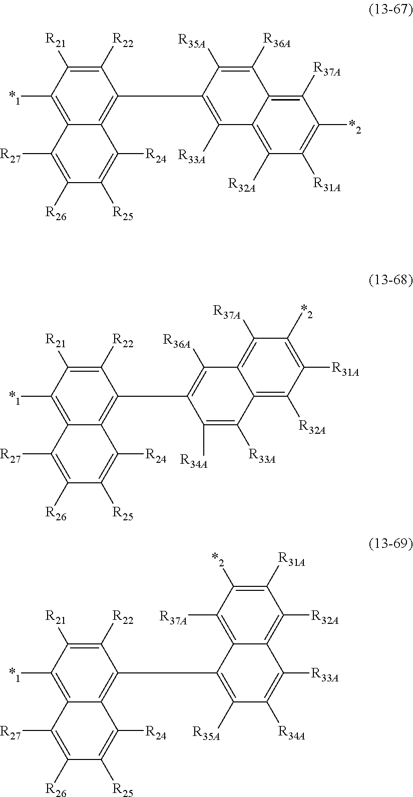

In the formulae (13-1) to (13-69), R₁₁ to R₁₅, R₂₁ to R₂₇, R₃₁ to R₃₇, R_{11A} to R_{15A}, R_{21A} to R_{27A}, and R_{31A} to R_{37A} represent the same as R₁₁ to R₁₅, R₂₁ to R₂₇, R₃₁ to R₃₇, R_{11A} to R_{15A}, R_{21A} to R_{27A}, and R_{31A} to R_{37A}, respectively, in the formulae (11) to (13) and (11A) to (13A), *1 in the formulae (13-1) to (13-69) represents the position of bonding to *a in the formula (120), and *2 represents the position of bonding to *b in the formula (120).

In the compound according to the second exemplary embodiment, a compound represented by the formula (120) is preferably represented by one of the following formulae (121) to (131).

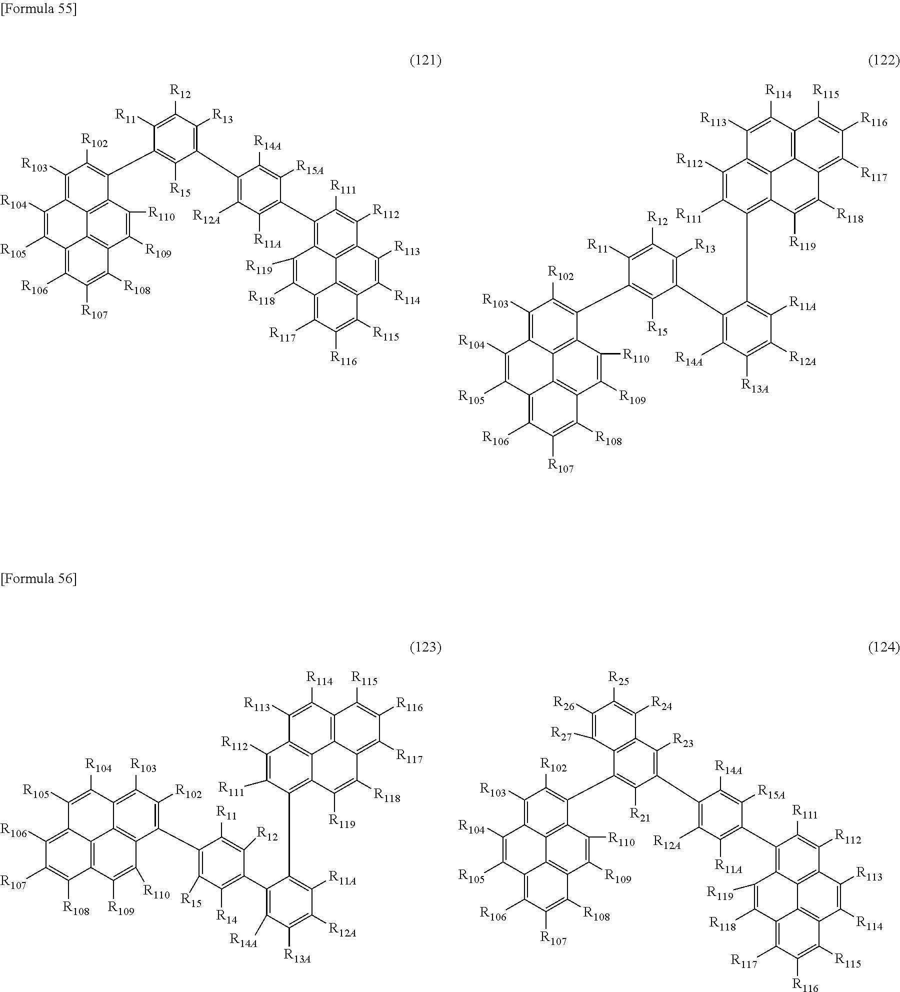

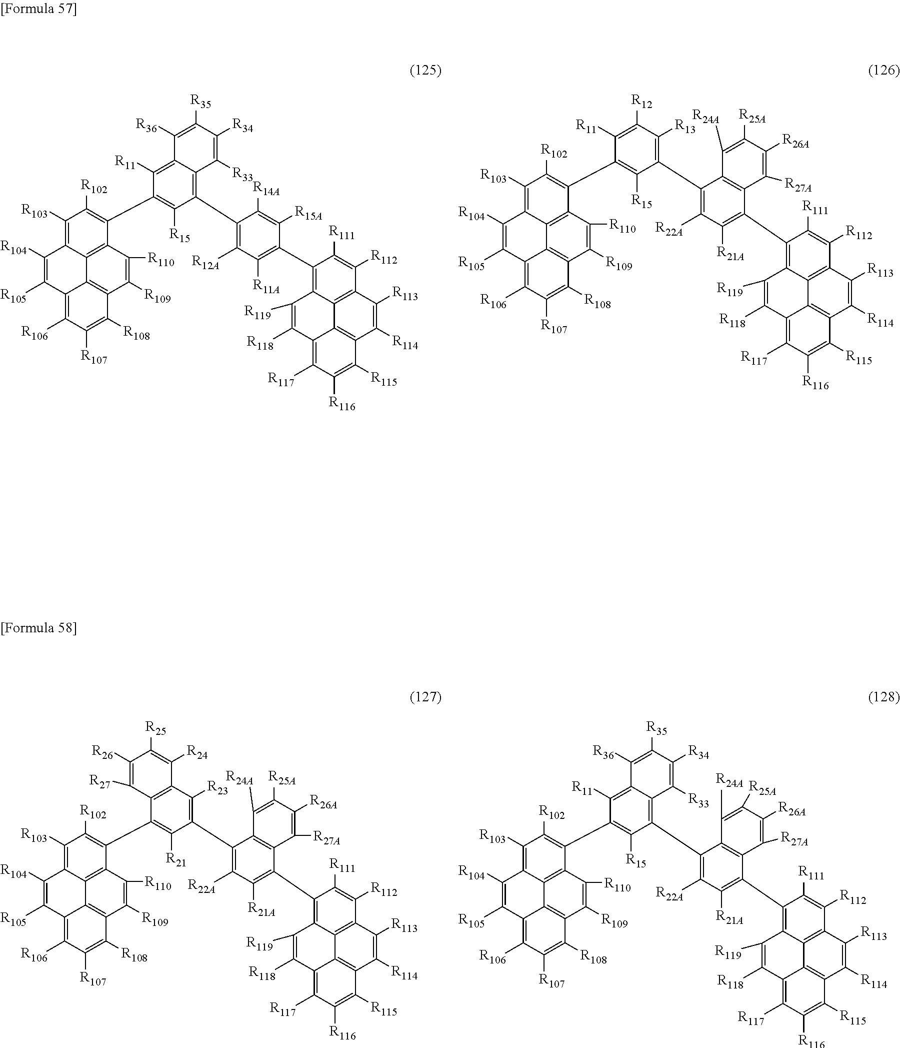

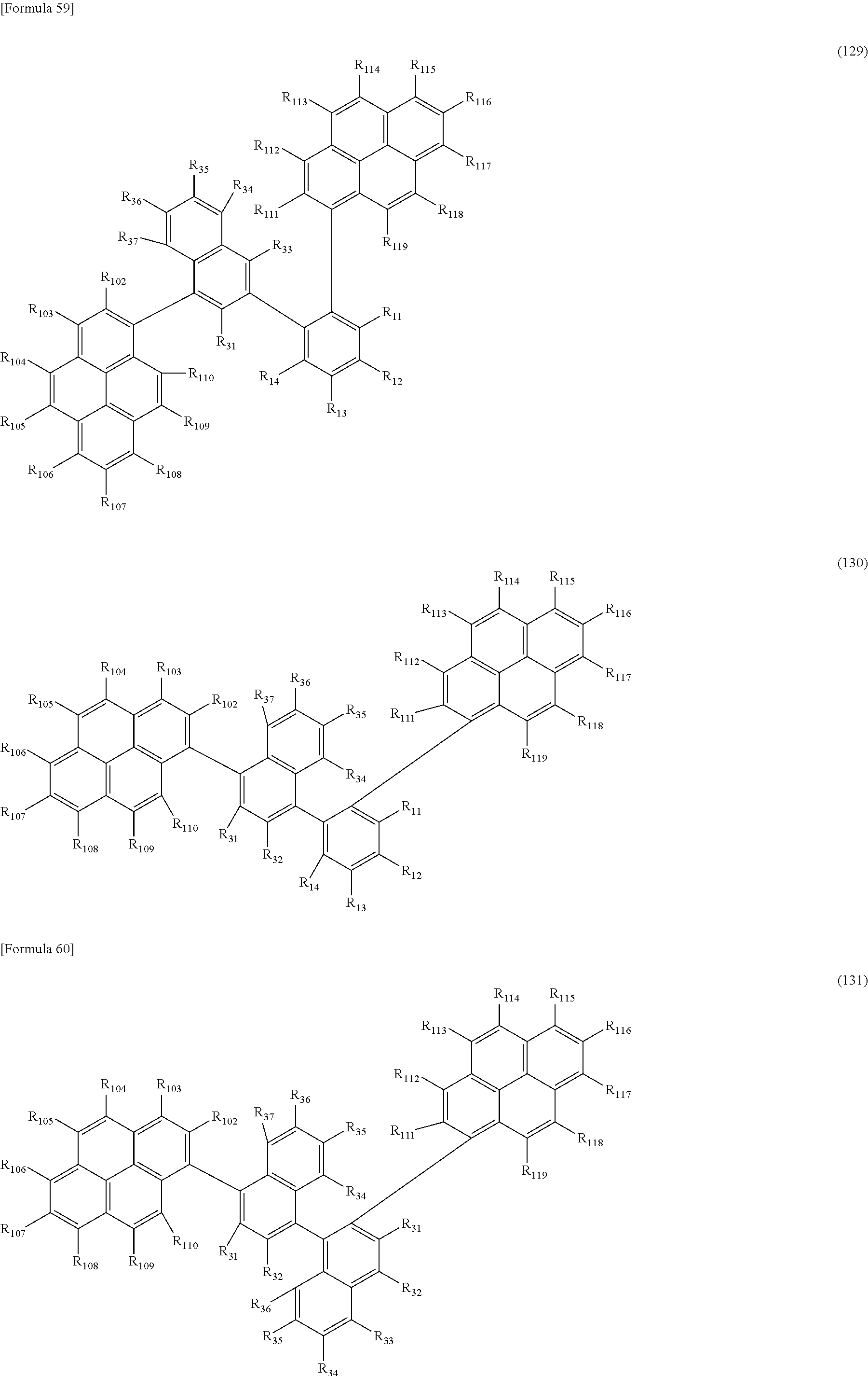

In the formulae (121) to (131),
R₁₁ to R₁₅, R₂₁ to R₂₇, R₃₁ to R₃₇, R_{11A} to R_{15A}, R_{21A} to R_{27A}, and R_{31A} to R_{37A} represent the same as R₁₁ to R₁₅, R₂₁ to R₂₇, R₃₁ to R₃₇, R_{11A} to R_{15A}, R_{21A} to R_{27A}, and R_{31A} to R_{37A}, respectively, in the formulae (11) to (13) and (11A) to (13A), and R₁₀₂ to R₁₁₉ represent the same as R₁₀₂ to R₁₁₉, respectively, in the formula (120).

In the compound according to the second exemplary embodiment, R₁₁ to R₁₅, R₂₁ to R₂₇, R₃₁ to R₃₇, R_{11A} to R_{15A}, R_{21A} to R_{27A}, R_{31A} to R_{37A}, and R₁₀₂ to R₁₁₉ preferably each independently represent a hydrogen atom, a substituted or unsubstituted alkyl group having 1 to 30 carbon atoms, a substituted or unsubstituted cycloalkyl group having 1 to 30 carbon atoms, a group represented by —Si(Rx)(Ry)(Rz), a substituted or unsubstituted aryl group having 6 to 30 ring carbon atoms, or a substituted or unsubstituted heterocyclic group having 5 to 30 ring atoms. Rx, Ry, and Rz in the —Si(Rx)(Ry)(Rz) preferably each independently represent an unsubstituted alkyl group having 1 to 30 carbon atoms or an unsubstituted aryl group having 6 to 30 ring carbon atoms.

In the compound according to the second exemplary embodiment, R₁₁ to R₁₅, R₂₁ to R₂₇, R₃₁ to R₃₇, R_{11A} to R_{15A}, R_{21A} to R_{27A}, R_{31A} to R_{37A}, and R₁₀₂ to R₁₁₉ more preferably each independently represent a hydrogen atom, a substituted or unsubstituted alkyl group having 1 to 18 carbon atoms, a substituted or unsubstituted cycloalkyl group having 1 to 18 carbon atoms, a group represented by —Si(Rx)(Ry)(Rz), a substituted or unsubstituted aryl group having 6 to 18 ring carbon atoms, or a substituted or unsubstituted heterocyclic group having 5 to 18 ring atoms. Rx, Ry, and Rz in the —Si(Rx)(Ry)(Rz) more preferably each independently represent an unsubstituted alkyl group having 1 to 18 carbon atoms or an unsubstituted aryl group having 6 to 18 ring carbon atoms.

In the compound according to the second exemplary embodiment, R₁₁ to R₁₅, R₂₁ to R₂₇, R₃₁ to R₃₇, R_{11A} to R_{15A}, R_{21A} to R_{27A}, R_{31A} to R_{37A}, and R₁₀₂ to R₁₁₉ preferably each independently represent a hydrogen atom, a substituted or unsubstituted alkyl group having 1 to 18 carbon atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, a substituted or unsubstituted 9,9-diphenylfluorenyl group, a substituted or unsubstituted pyridyl group, a substituted or unsubstituted pyrimidinyl group, a substituted or unsubstituted triazinyl group, a substituted or unsubstituted quinolyl group, a substituted or unsubstituted isoquinolyl group, a substituted or unsubstituted quinazolinyl group, a substituted or unsubstituted benzimidazolyl group, a substituted or unsubstituted phenanthrolinyl group, a substituted or unsubstituted 1-carbazolyl group, a substituted or unsubstituted 2-carbazolyl group, a substituted or unsubstituted 3-carbazolyl group, a substituted or unsubstituted 4-carbazolyl group, a substituted or unsubstituted 9-carbazolyl group, a substituted or unsubstituted benzocarbazolyl group, a substituted or unsubstituted azacarbazolyl group, a substituted or unsubstituted diazacarbazolyl group, a substituted or unsubstituted dibenzofuranyl group, a substituted or unsubstituted naphthobenzofuranyl group, a substituted or unsubstituted azadibenzofuranyl group, a substituted or unsubstituted diazadibenzofuranyl group, a substituted or unsubstituted dibenzothiophenyl group, a substituted or unsubstituted naphthobenzothiophenyl group, a substituted or unsubstituted azadibenzothiophenyl group, or a substituted or unsubstituted diazadibenzothiophenyl group.

In the compound according to the second exemplary embodiment, R₁₁ to R₁₅, R₂₁ to R₂₇, R₃₁ to R₃₇, R_{11A} to R_{15A}, R_{21A} to R_{27A}, and R_{31A} to R_{37A}, and R₁₀₂ to R₁₁₉ preferably each independently represent a hydrogen atom, a substituted or unsubstituted alkyl group having 1 to 6 carbon atoms, a substituted or unsubstituted phenyl group, a substituted or unsubstituted naphthyl group, a substituted or unsubstituted phenanthryl group, a substituted or unsubstituted fluorenyl group, a substituted or unsubstituted dibenzofuranyl group, a substituted or unsubstituted dibenzothiophenyl group, a substituted or unsubstituted 9,9-dimethylfluorenyl group, or a substituted or unsubstituted 9,9-diphenylfluorenyl group.

In the compound according to the second exemplary embodiment, R₁₁ to R₁₅, R₂₁ to R₂₇, R₃₁ to R₃₇, R_{11A} to R_{15A}, R_{21A} to R_{27A}, and R_{31A} to R_{37A}, and R₁₀₂ to R₁₁₉ more preferably each independently represent a hydrogen atom, an unsubstituted alkyl group having 1 to 6 carbon atoms, an unsubstituted phenyl group, an unsubstituted naphthyl group, an unsubstituted phenanthryl group, a substituted or unsubstituted fluorenyl group, an unsubstituted dibenzofuranyl group, an unsubstituted dibenzothiophenyl group, an unsubstituted 9,9-dimethylfluorenyl group, or an unsubstituted 9,9-diphenylfluorenyl group.

In the compound according to the second exemplary embodiment, R₁₁ to R₁₅, R₂₁ to R₂₇, R₃₁ to R₃₇, R_{11A} to R_{15A}, R_{21A} to R_{27A}, and R_{31A} to R_{37A} preferably each represent a hydrogen atom.

In the compound according to the second exemplary embodiment, R₁₀₂ to R₁₁₉ preferably each represent a hydrogen atom.

In the compound according to the second exemplary embodiment, a substituent for “substituted or unsubstituted” in R₁₁ to R₁₅, R₂₁ to R₂₇, R₃₁ to R₃₇, R_{11A} to R_{15A}, R_{21A} to R_{27A}, R_{31A} to R_{37A}, and R₁₀₂ to R₁₁₉ is preferably each independently a hydrogen atom, a substituted or unsubstituted alkyl group having 1 to 18 carbon atoms, a substituted or unsubstituted phenyl group, a substituted or unsubstituted naphthyl group, a substituted or unsubstituted phenanthryl group, a substituted or unsubstituted fluorenyl group, a substituted or unsubstituted dibenzofuranyl group, a substituted or unsubstituted dibenzothiophenyl group, a substituted or unsubstituted 9,9-dimethylfluorenyl group, or a substituted or unsubstituted 9,9-diphenylfluorenyl group.

In the compound according to the second exemplary embodiment, a substituent for “substituted or unsubstituted” in R₁₁ to R₁₅, R₂₁ to R₂₇, R₃₁ to R₃₇, R_{11A} to R_{15A}, R_{21A} to R_{27A}, R_{31A} to R_{37A}, and R₁₀₂ to R₁₁₉ is more preferably each independently a hydrogen atom, an unsubstituted alkyl group having 1 to 18 carbon atoms, an unsubstituted phenyl group, an unsubstituted naphthyl group, an unsubstituted phenanthryl group, a substituted or unsubstituted fluorenyl group, an unsubstituted dibenzofuranyl group, an unsubstituted dibenzothiophenyl group, an unsubstituted 9,9-dimethylfluorenyl group, or an unsubstituted 9,9-diphenylfluorenyl group.

The compound according to the second exemplary embodiment in the emitting layer can improve luminous efficiency.

### Method for Producing Compound According to Second Exemplary Embodiment

The compound according to the second exemplary embodiment (a compound represented by the formula (120)) can be produced by a known method. Furthermore, the compound according to the second exemplary embodiment can also be produced in accordance with a known method by using a known alternative reaction and raw materials suitable for the target compound.

Specific examples of the compound according to the second exemplary embodiment (a compound represented by the formula (120)) include the following compounds. However, the invention is not limited to these specific examples.

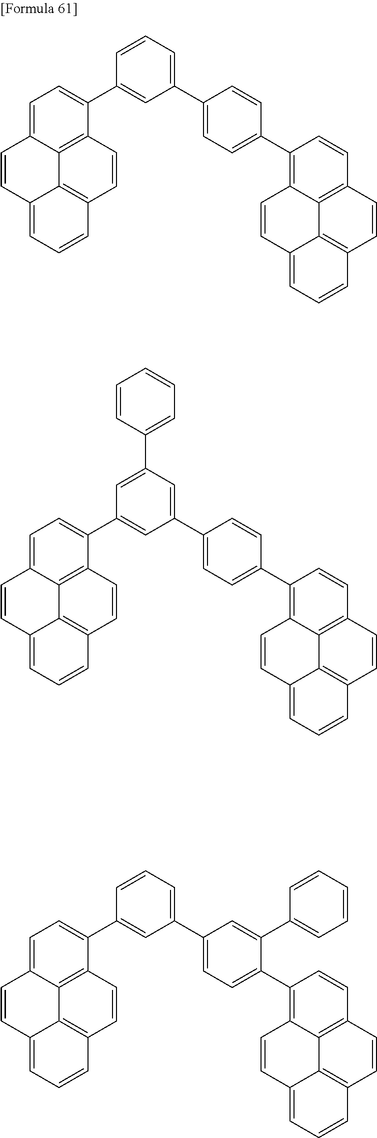

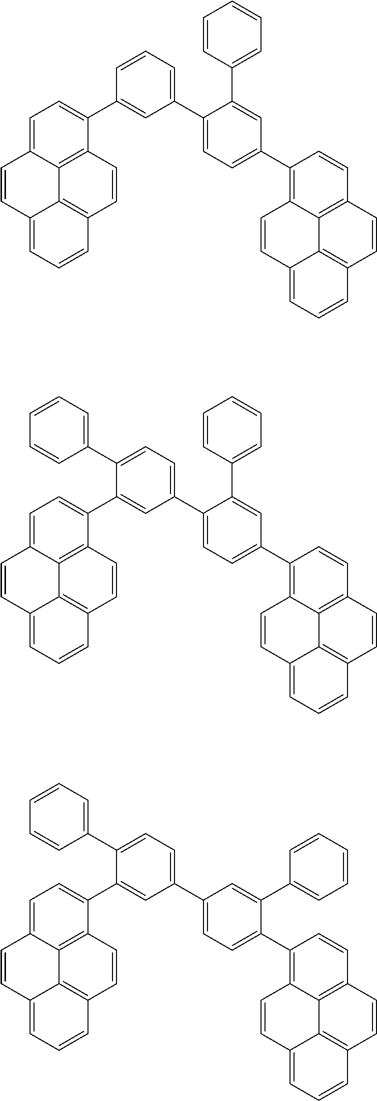

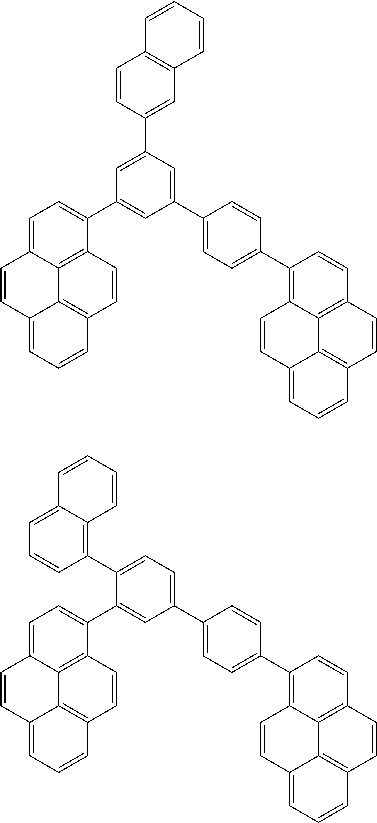

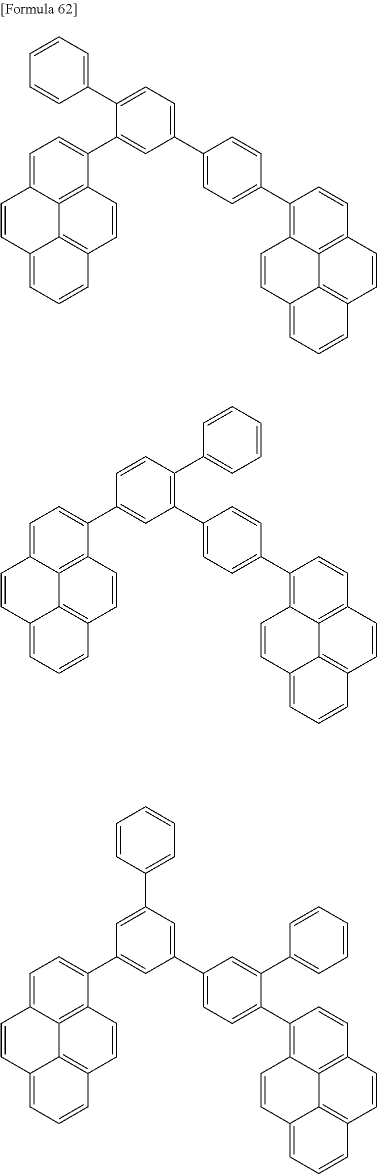

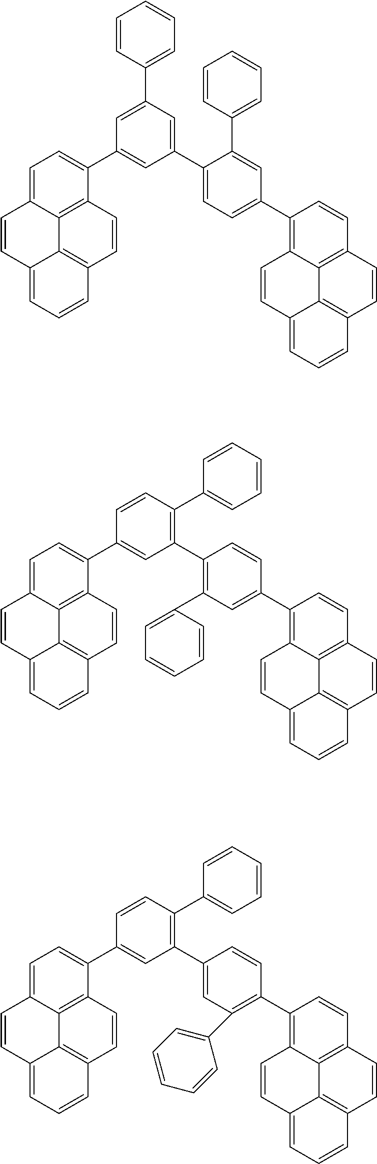

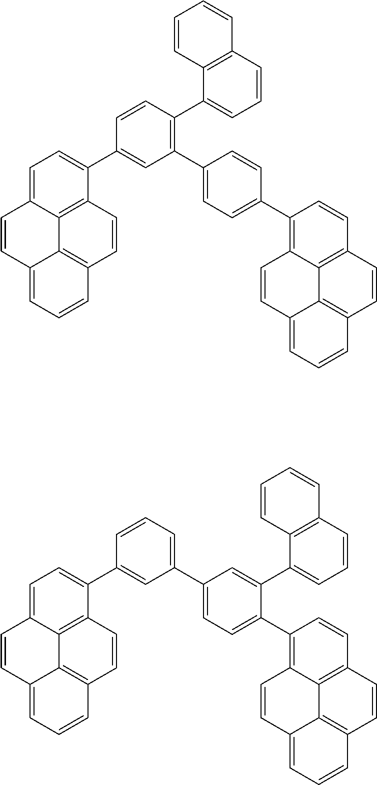

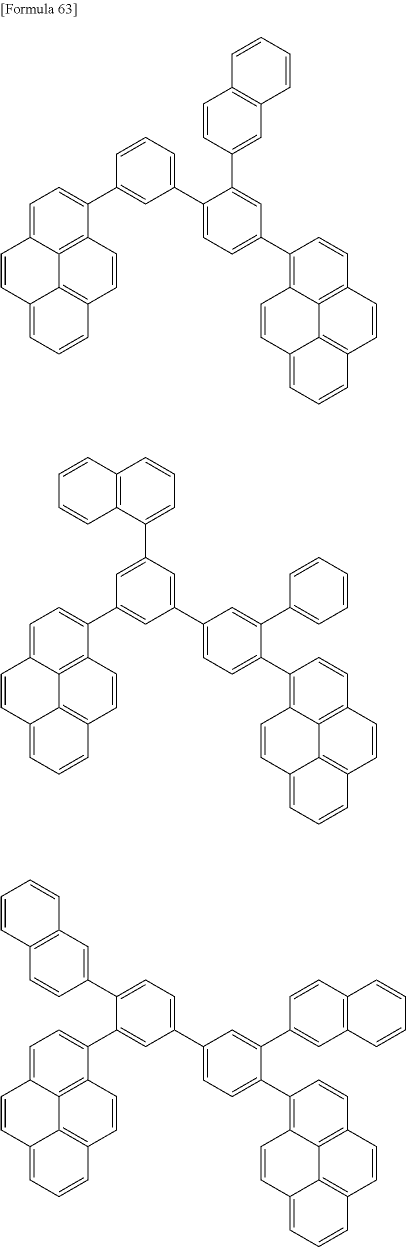

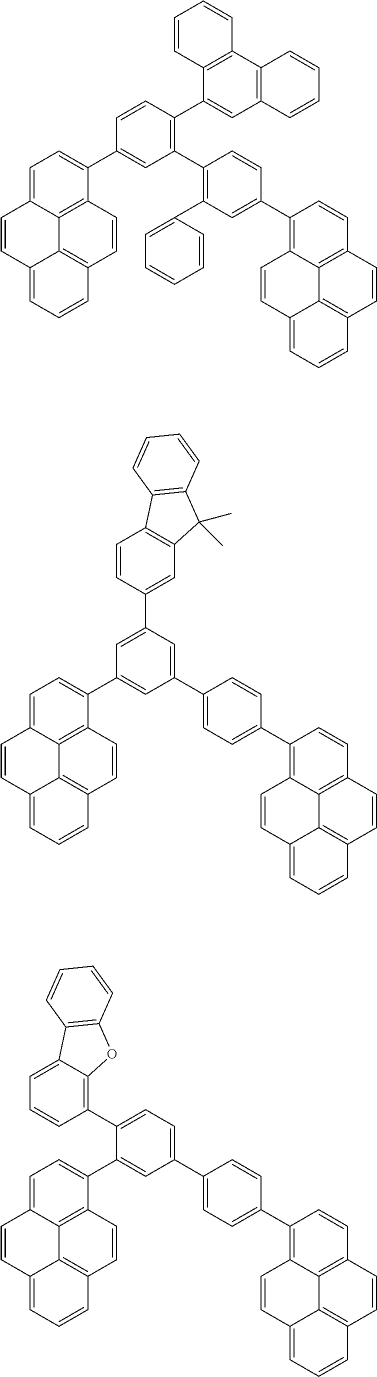

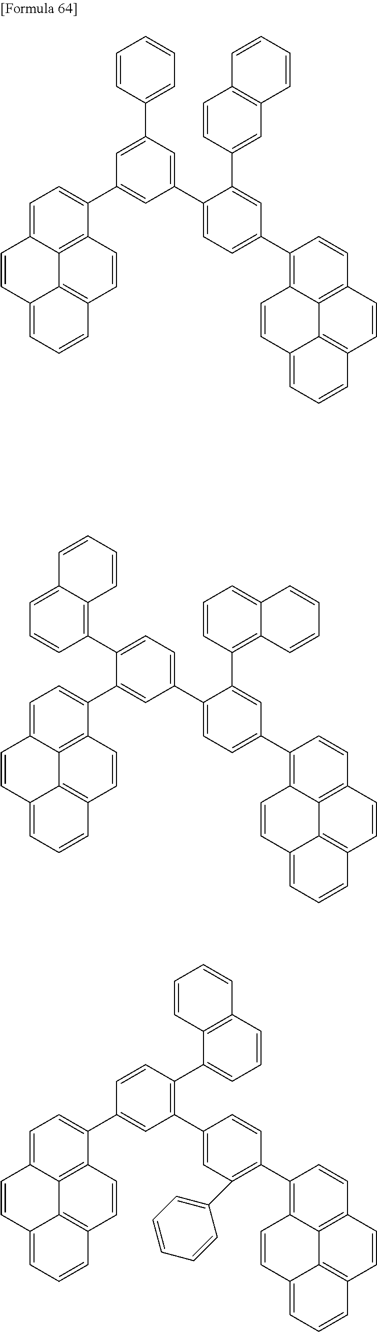

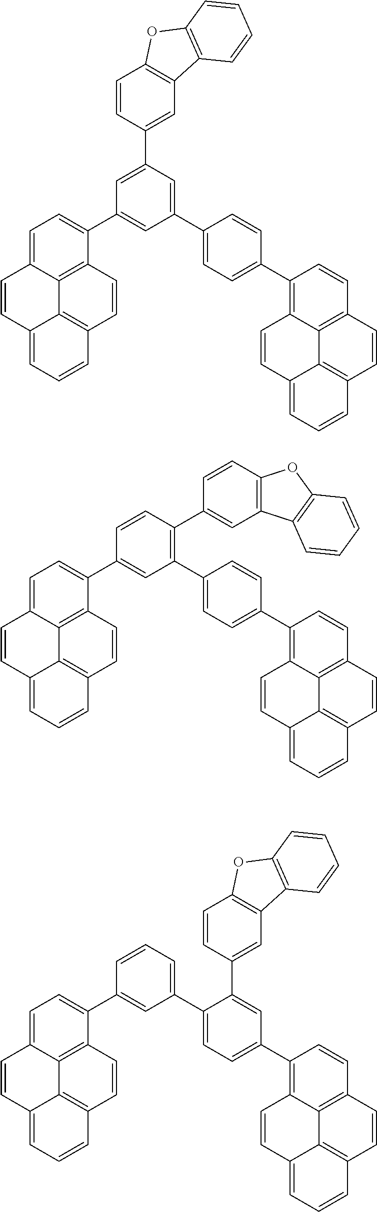

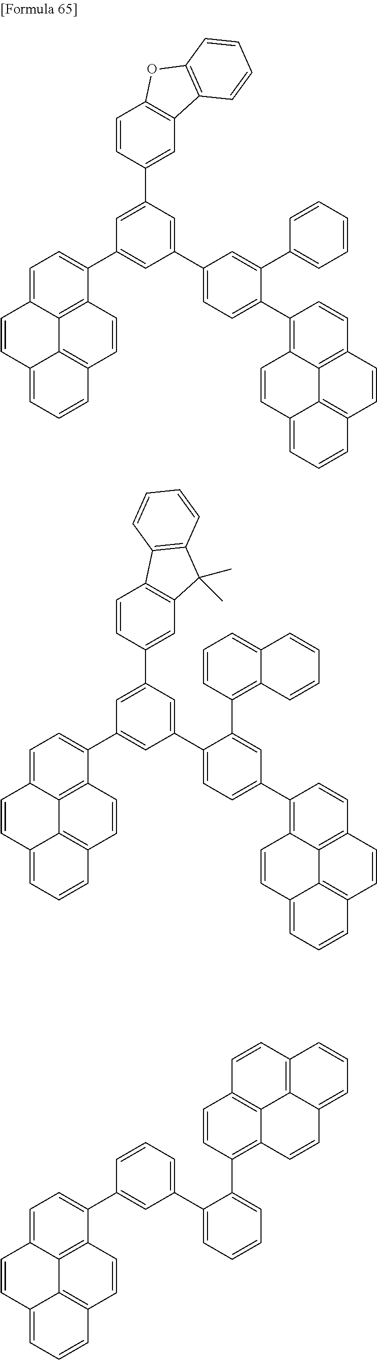

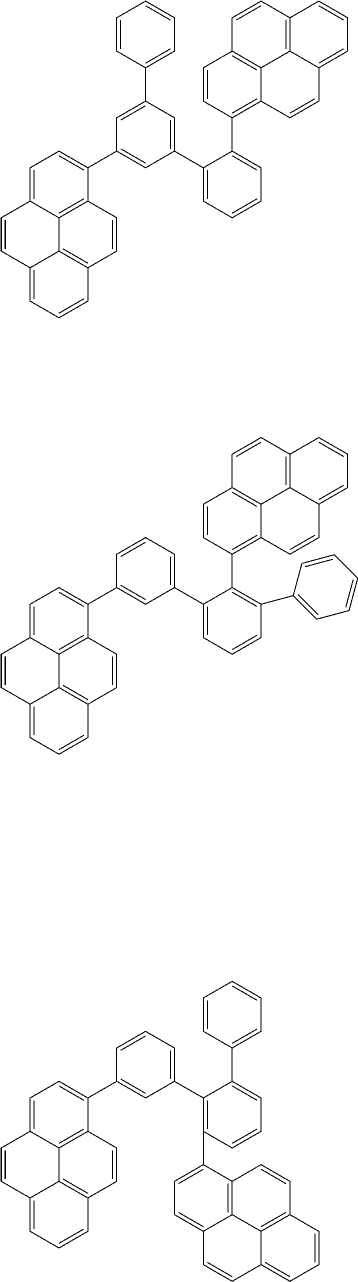

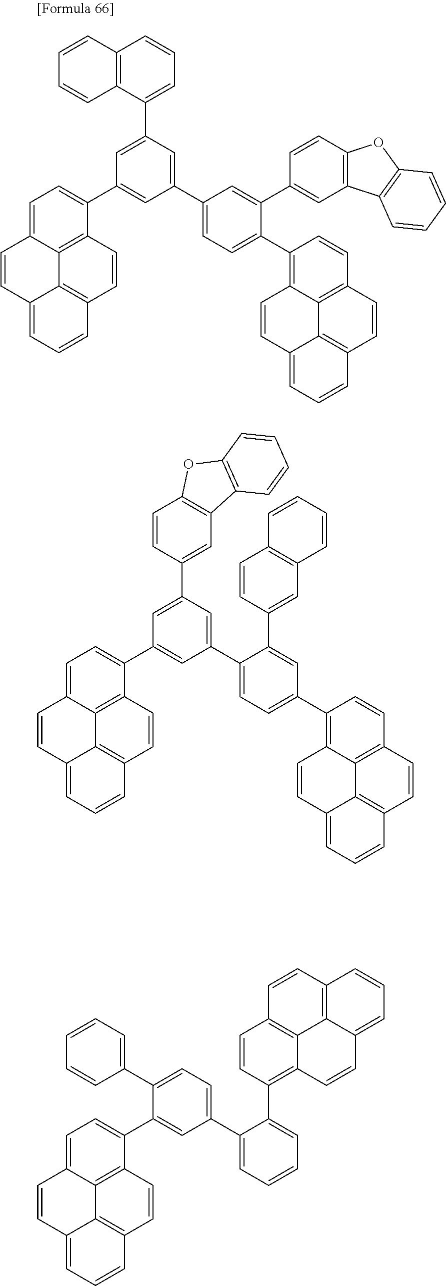

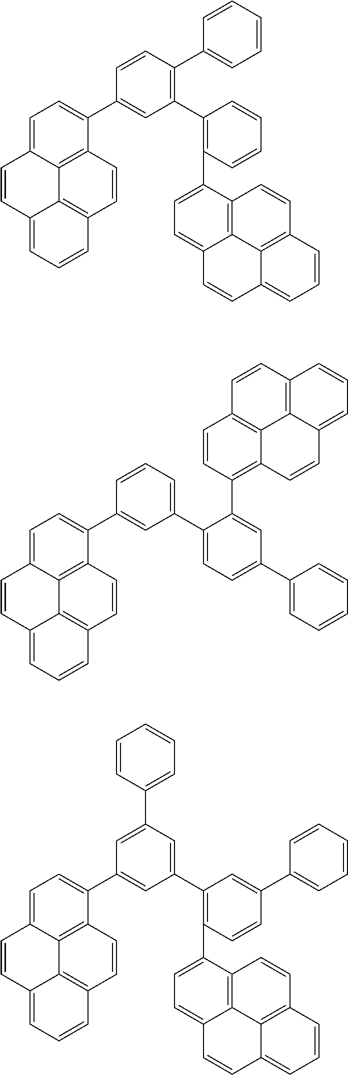

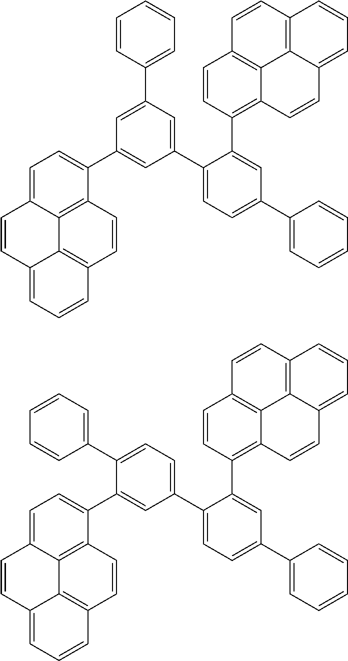

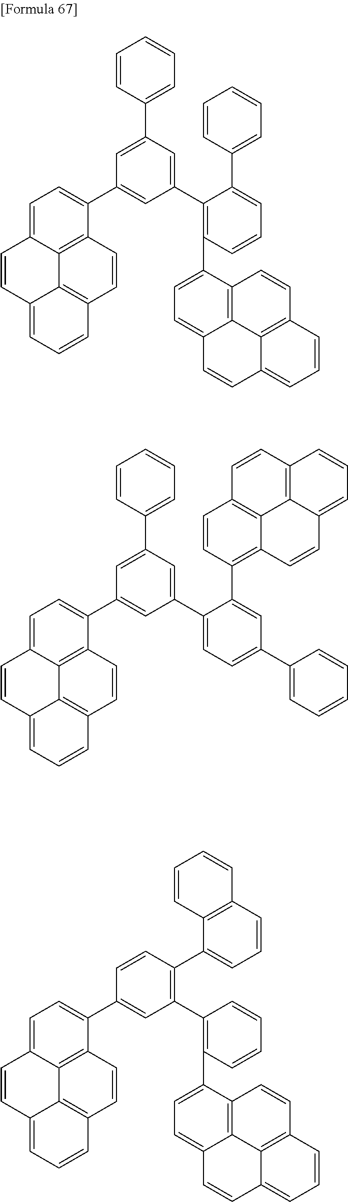

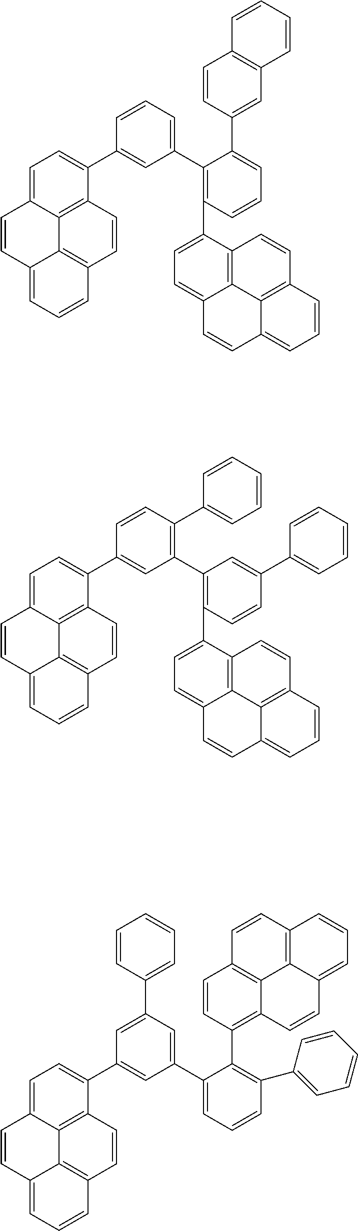

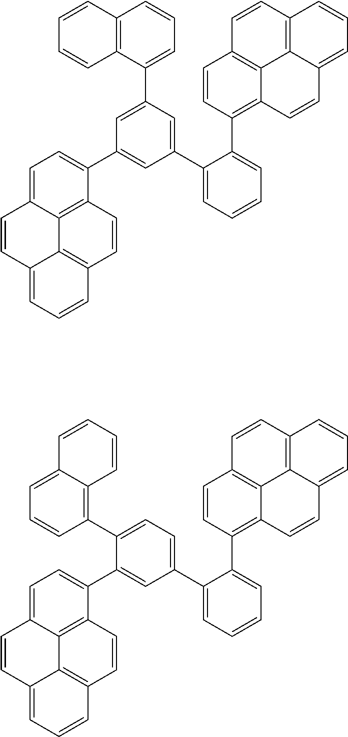

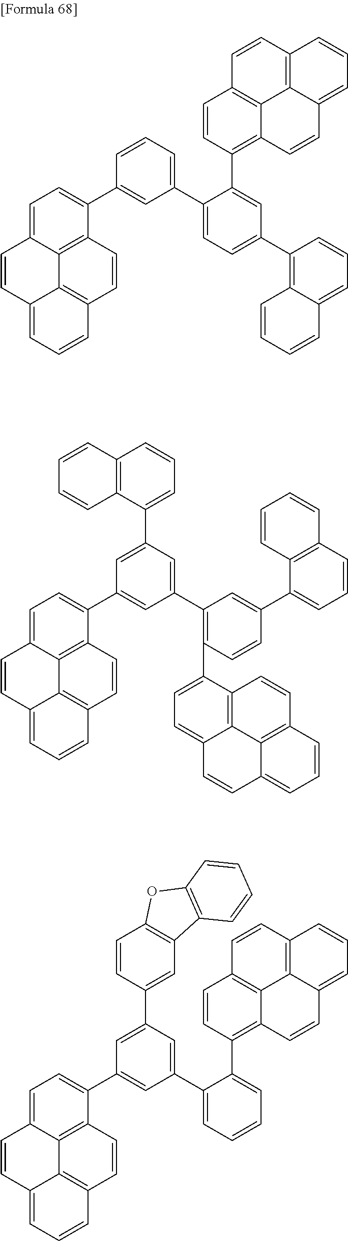

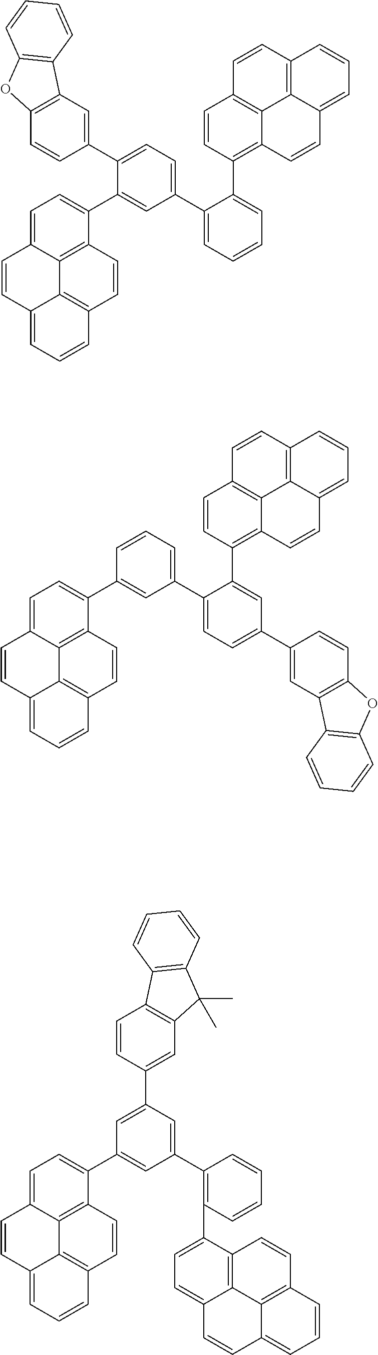

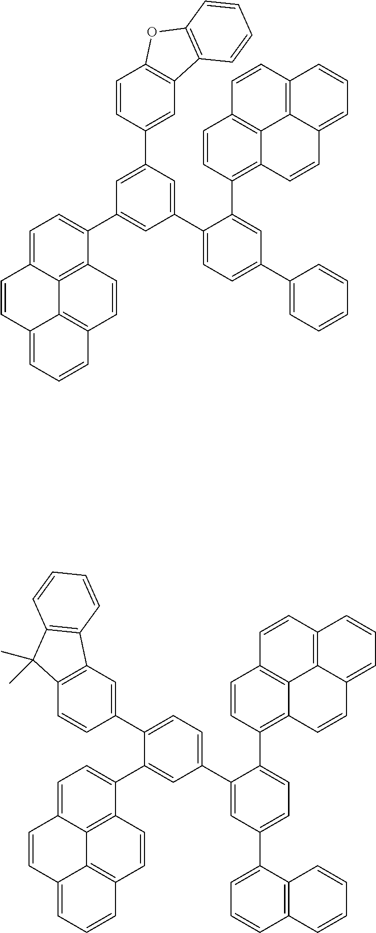

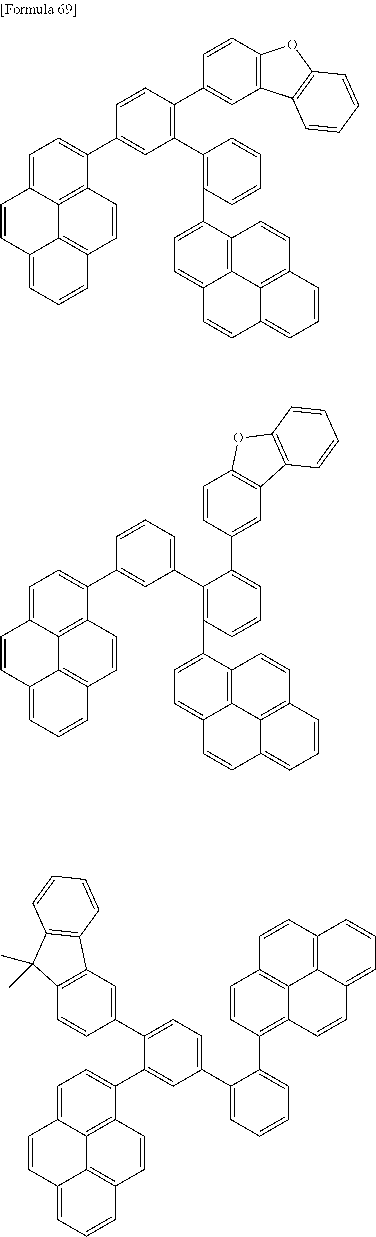

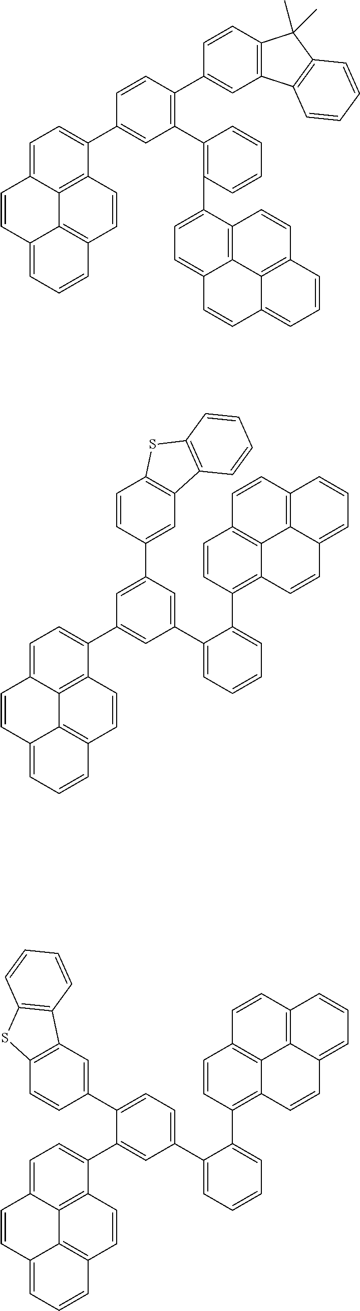

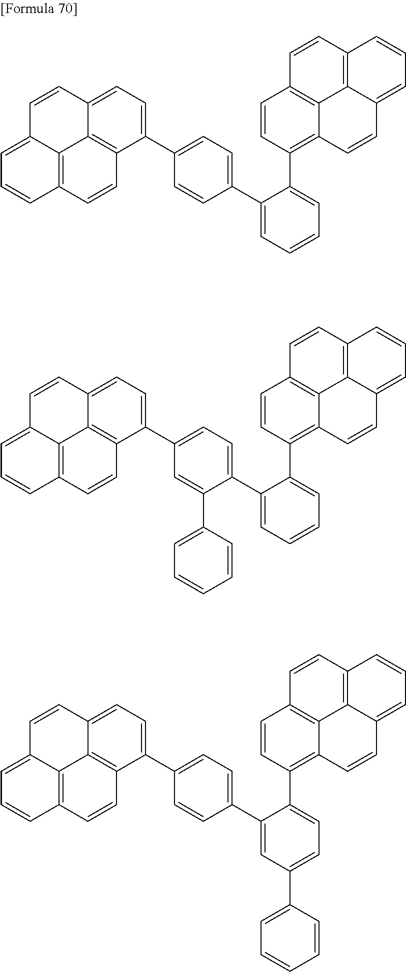

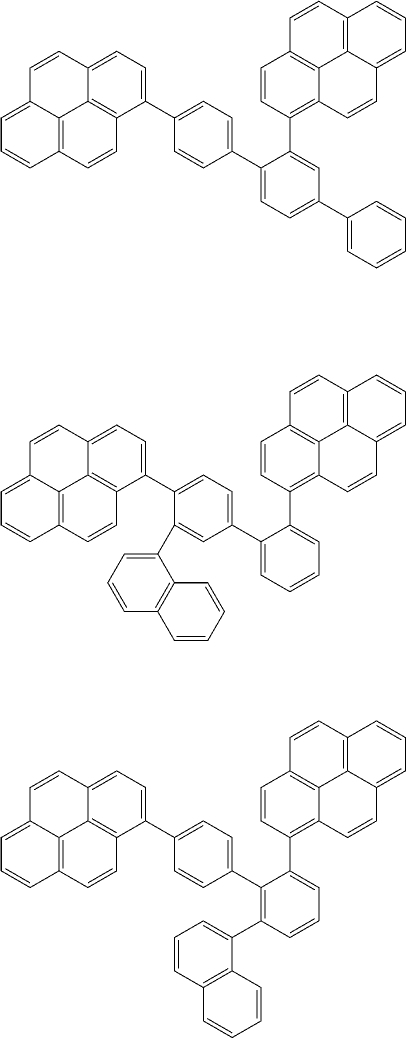

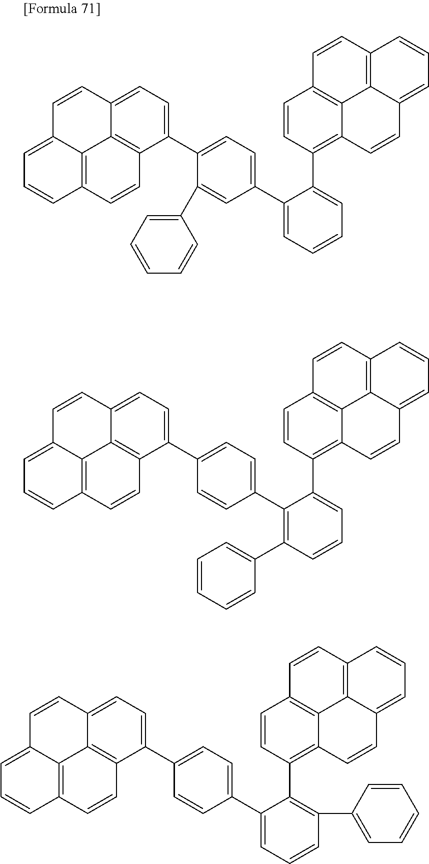

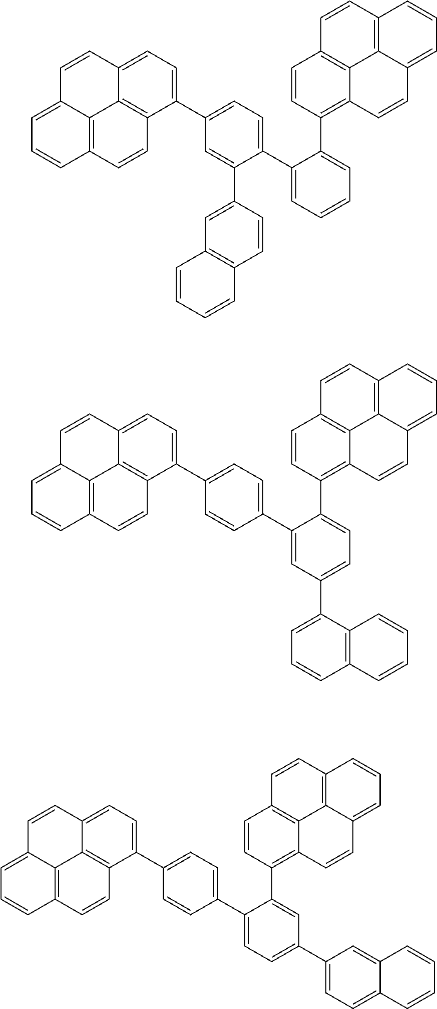

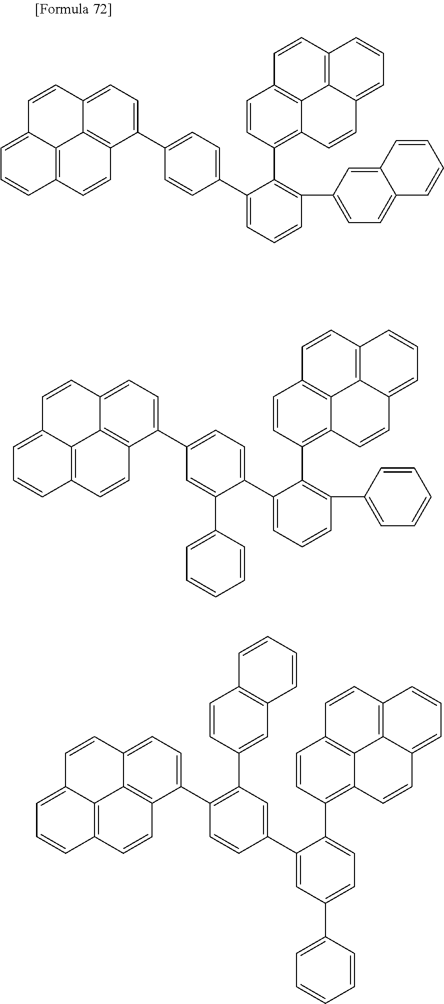

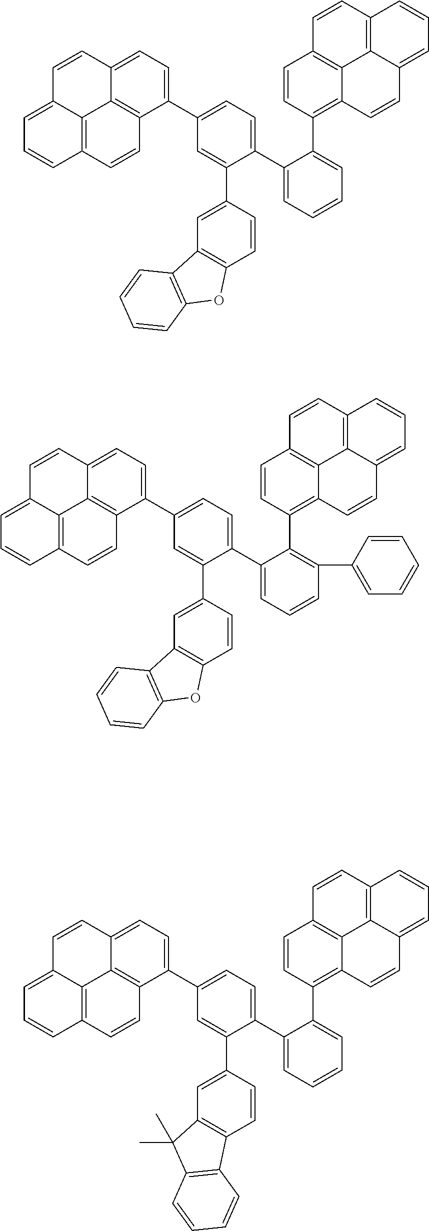

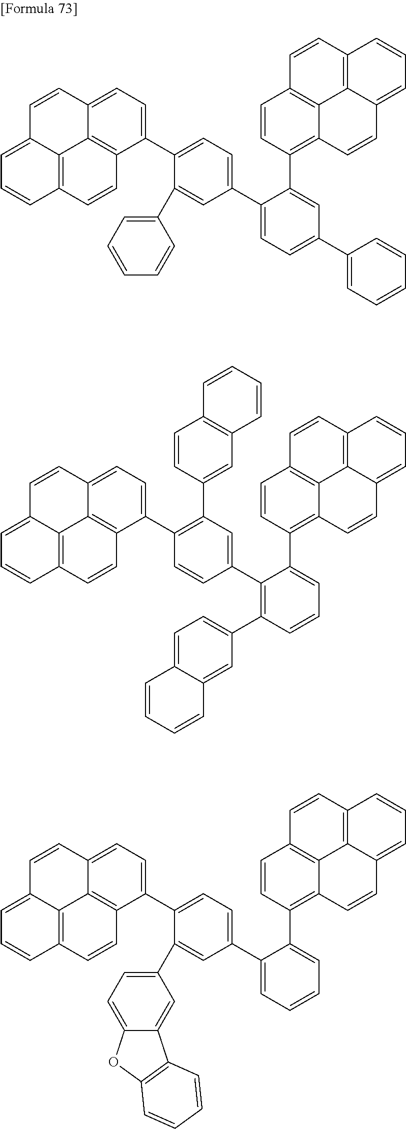

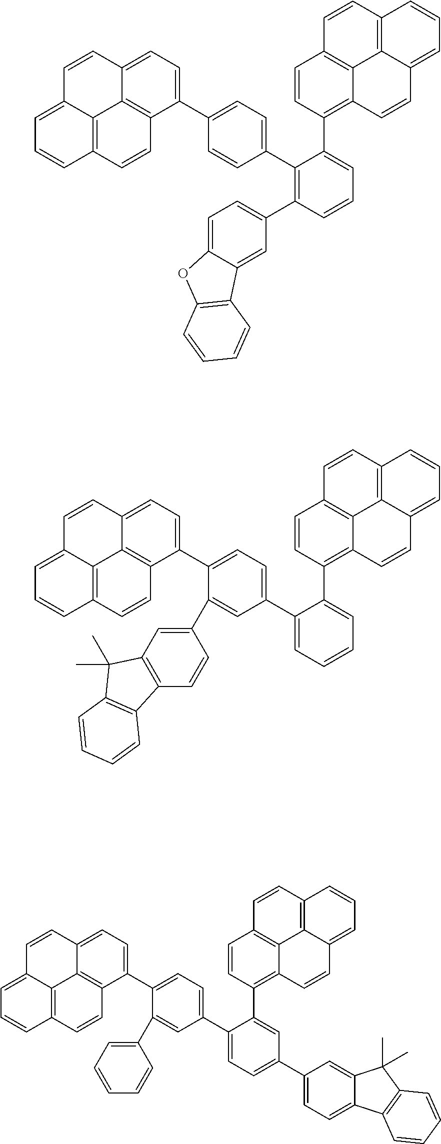

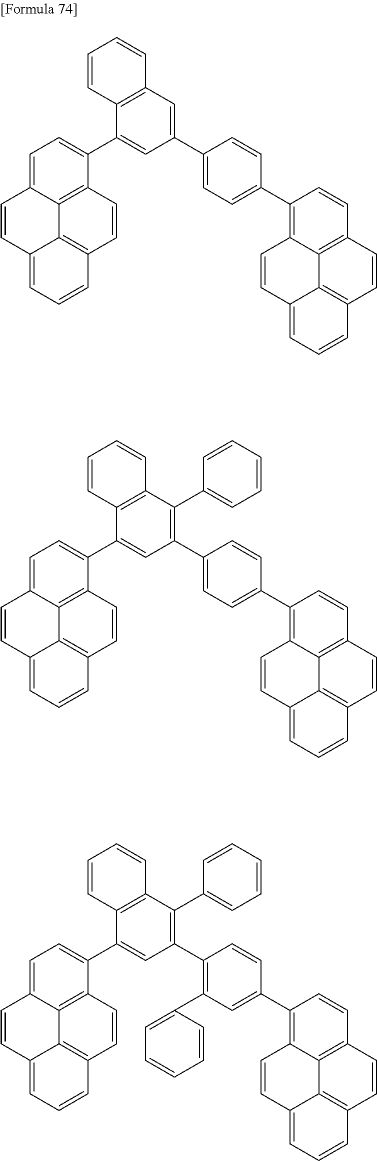

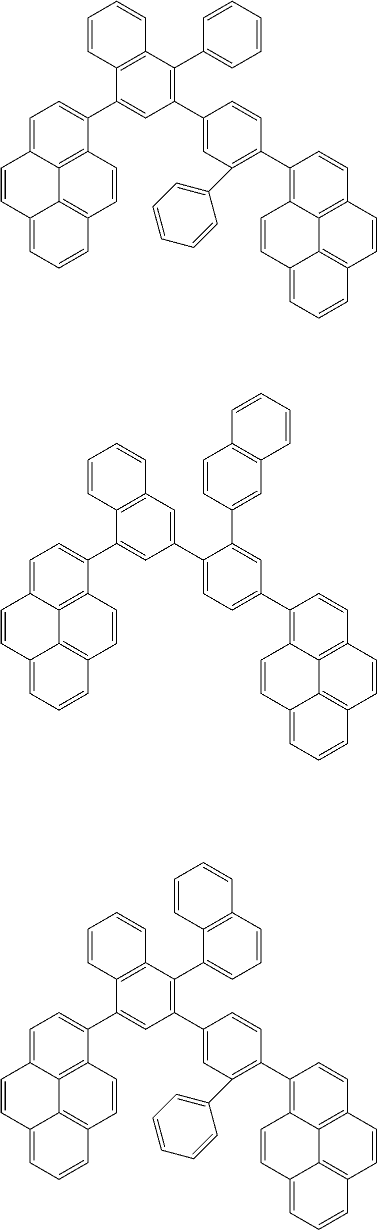

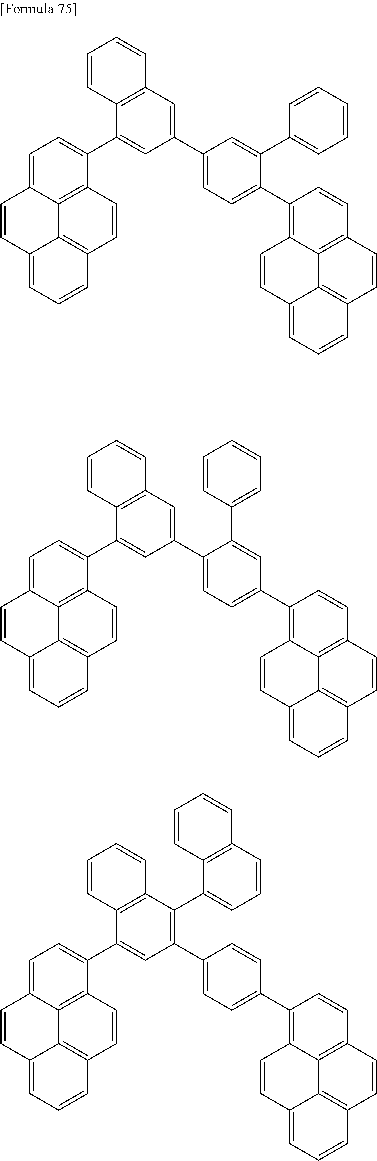

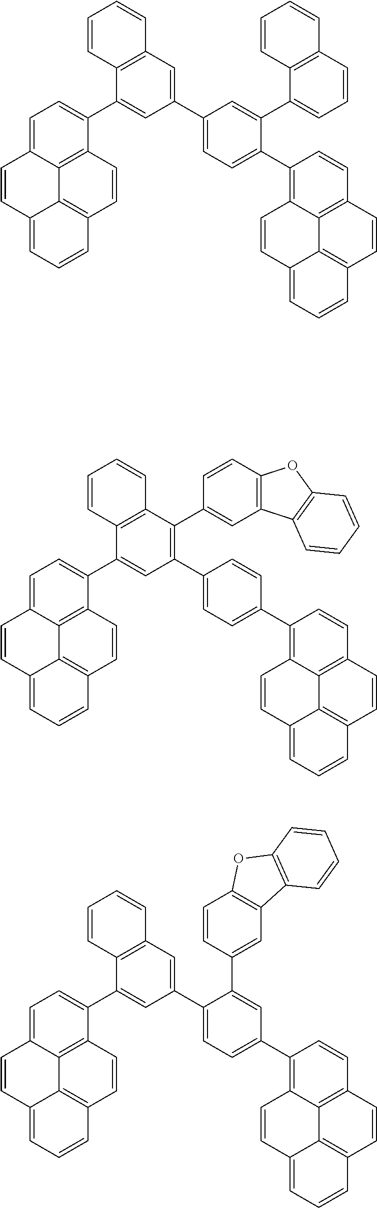

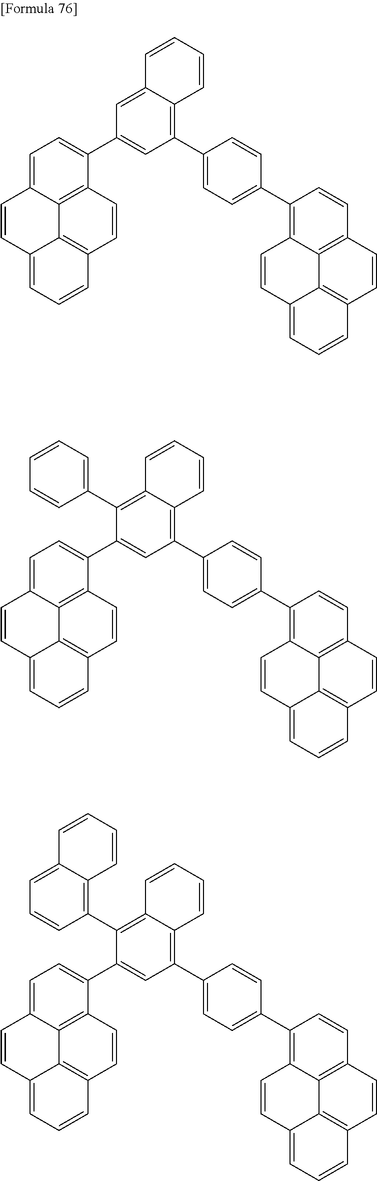

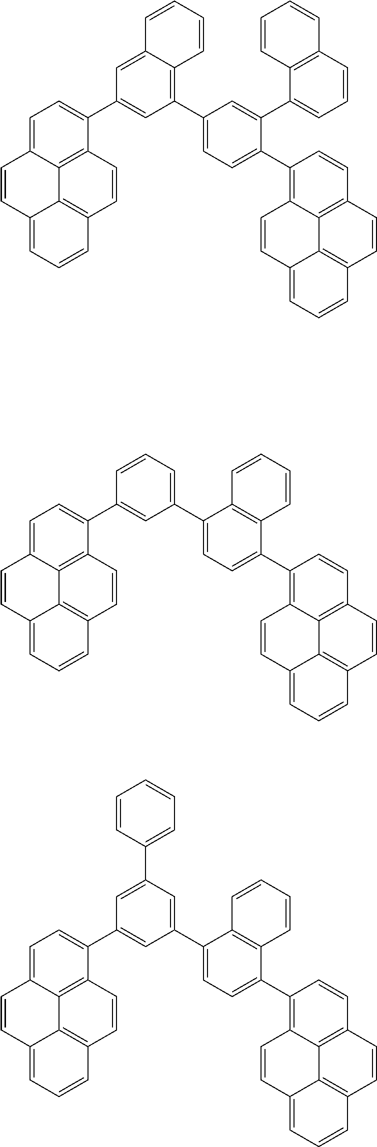

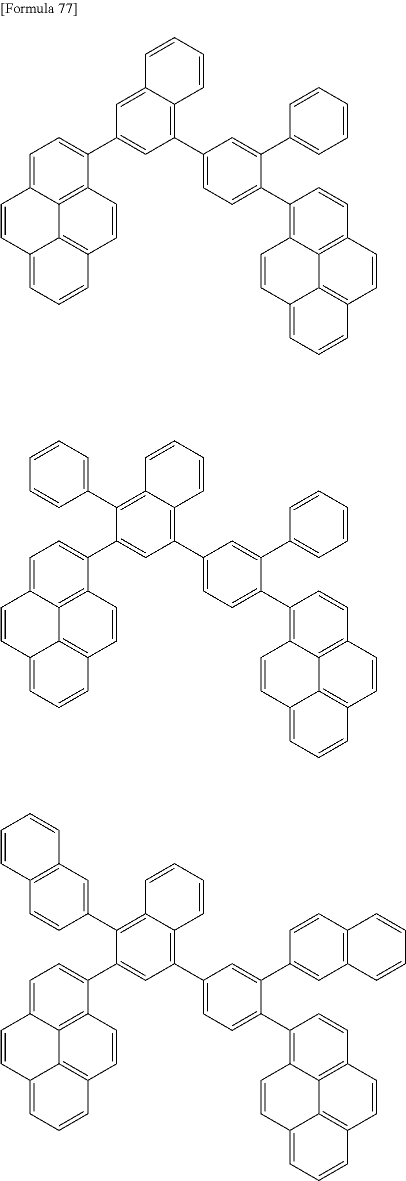

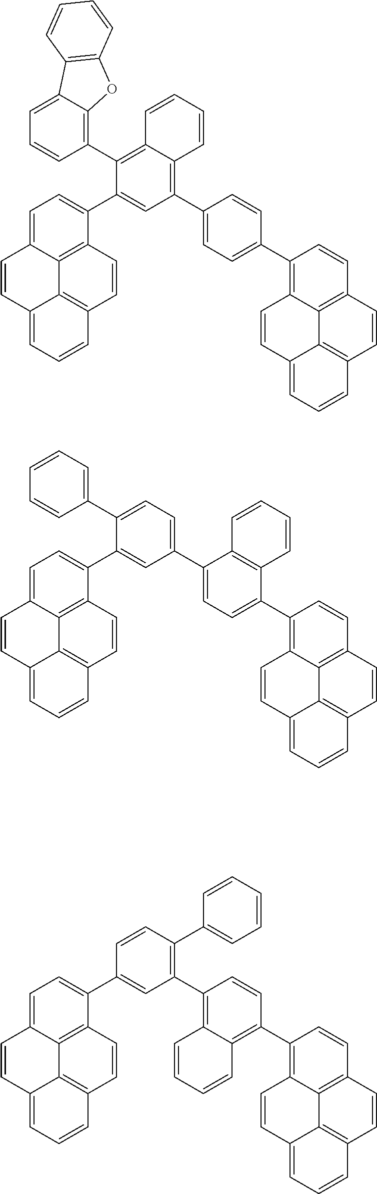

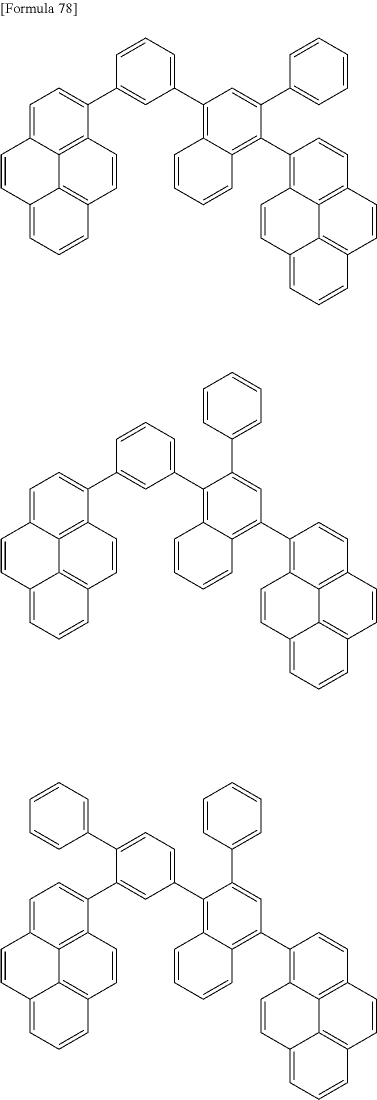

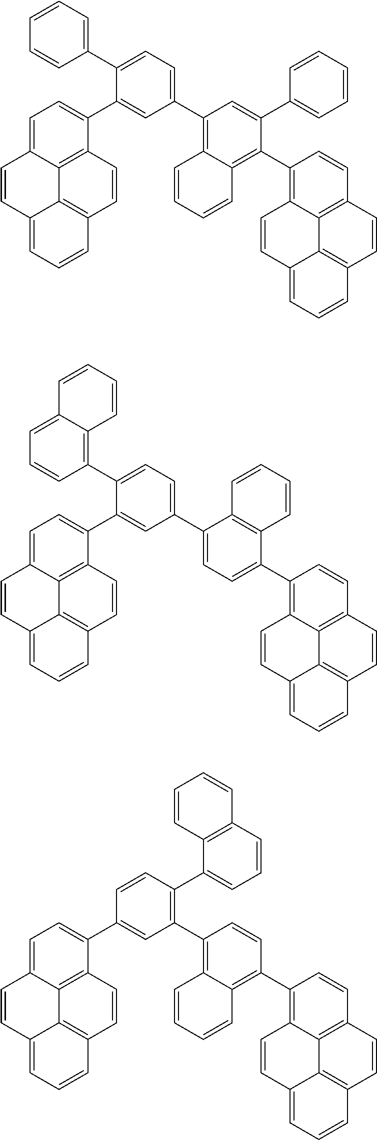

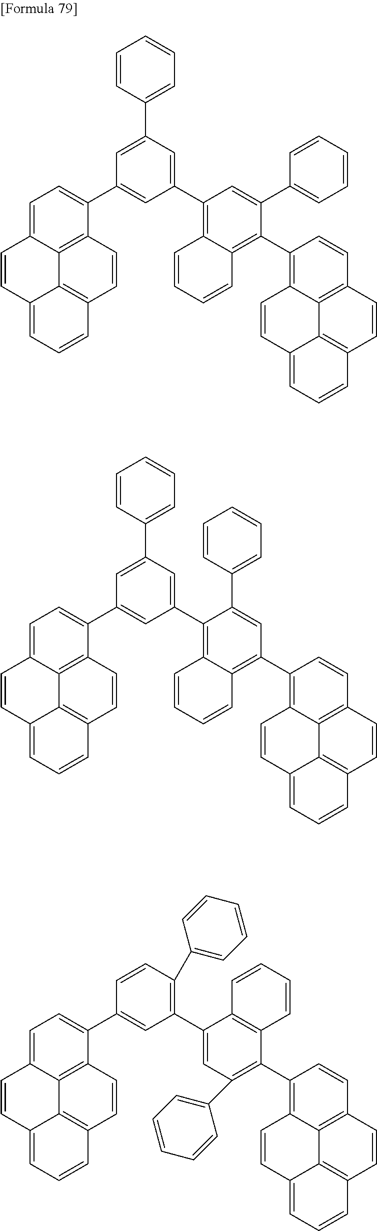

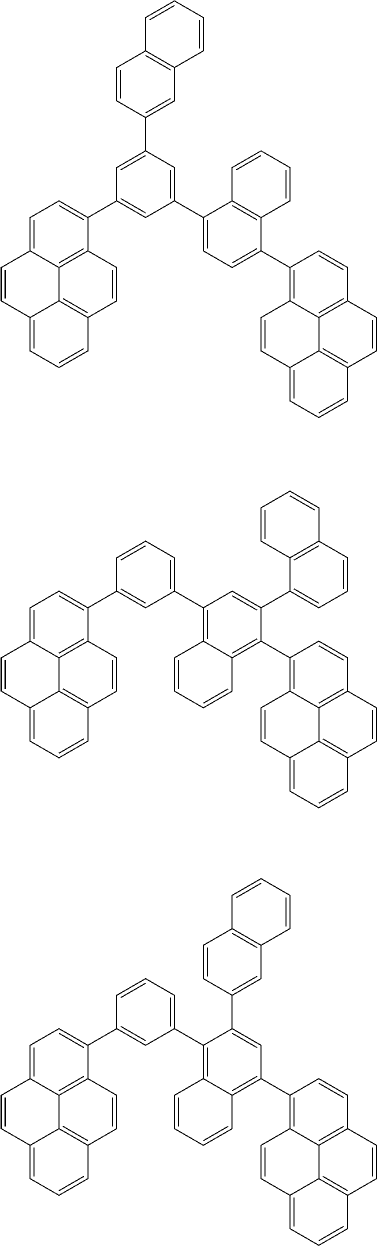

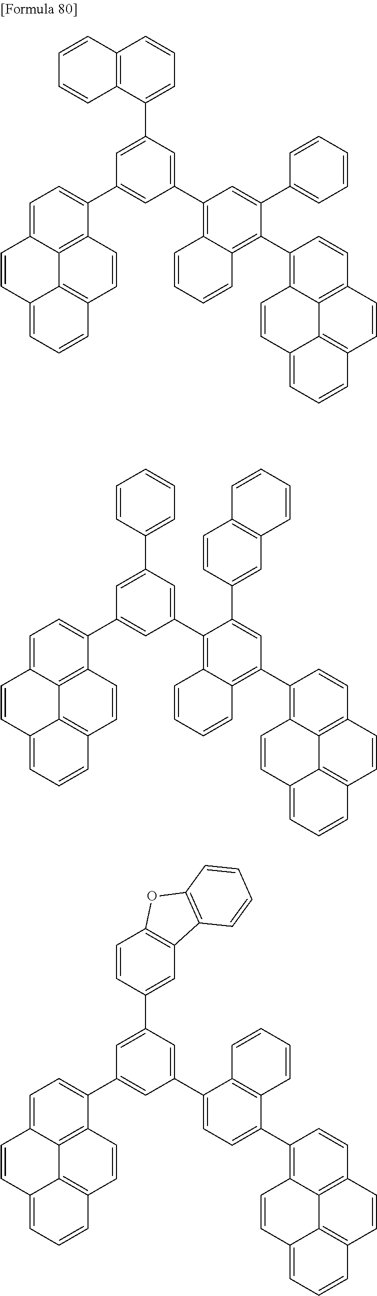

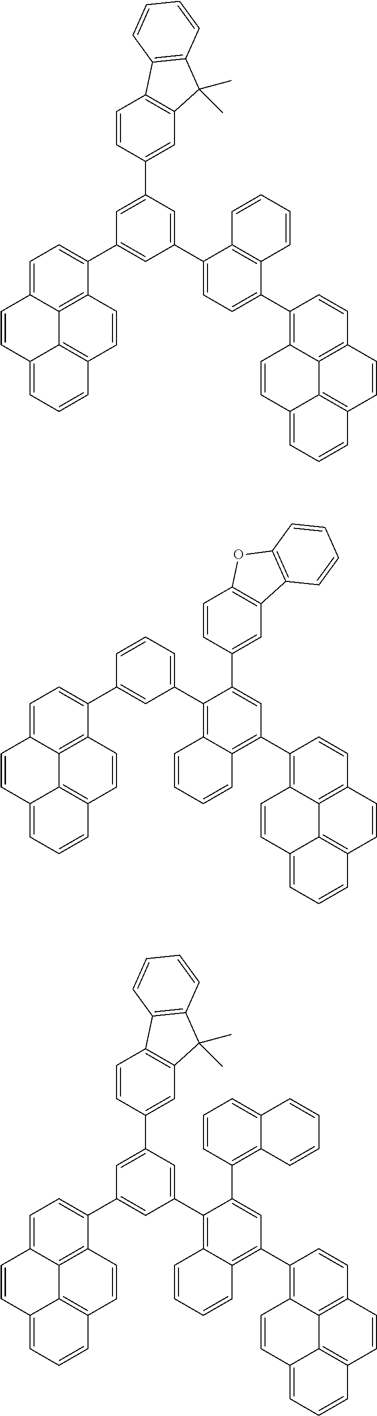

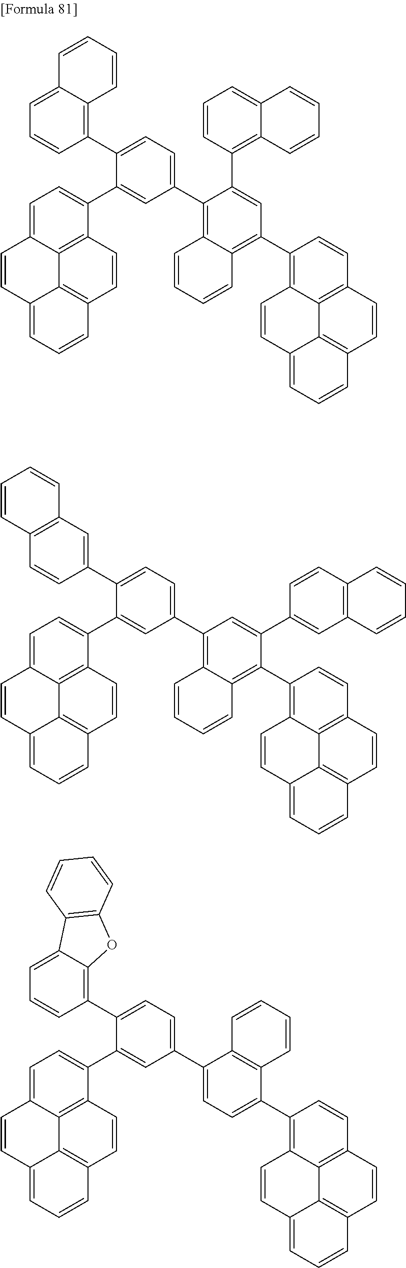

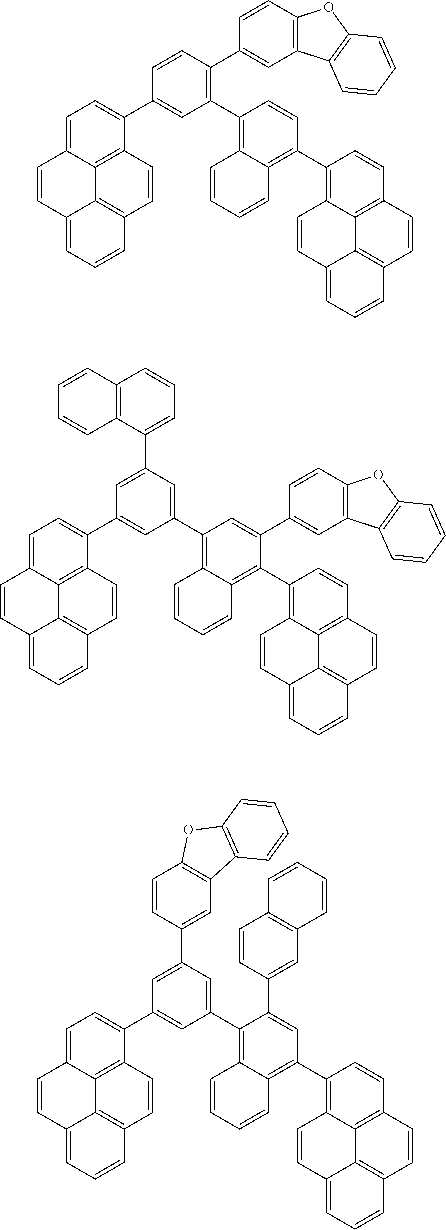 [SMILES: c1cc2ccc3ccc(-c4ccc(-c5ccc6oc7ccccc7c6c5)c(-c5ccc(-c6ccc7ccc8cccc9ccc6c7c89)c6ccccc56)c4)c4ccc(c1)c2c34 ; c1ccc2c(-c3cc(-c4cc(-c5ccc6oc7ccccc7c6c5)c(-c5ccc6ccc7cccc8ccc5c6c78)c5ccccc45)cc(-c4ccc5ccc6cccc7ccc4c5c67)c3)cccc2c1 ; c1ccc2cc(-c3cc(-c4ccc5ccc6cccc7ccc4c5c67)c4ccccc4c3-c3cc(-c4ccc5oc6ccccc6c5c4)cc(-c4ccc5ccc6cccc7ccc4c5c67)c3)ccc2c1]

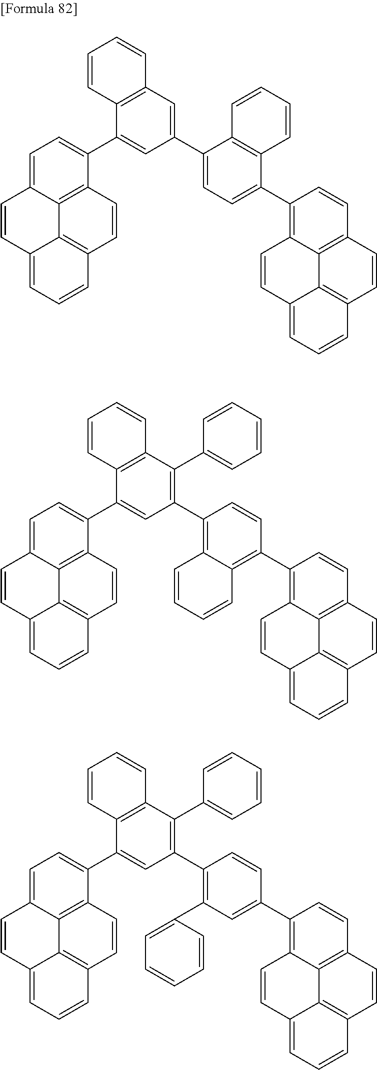

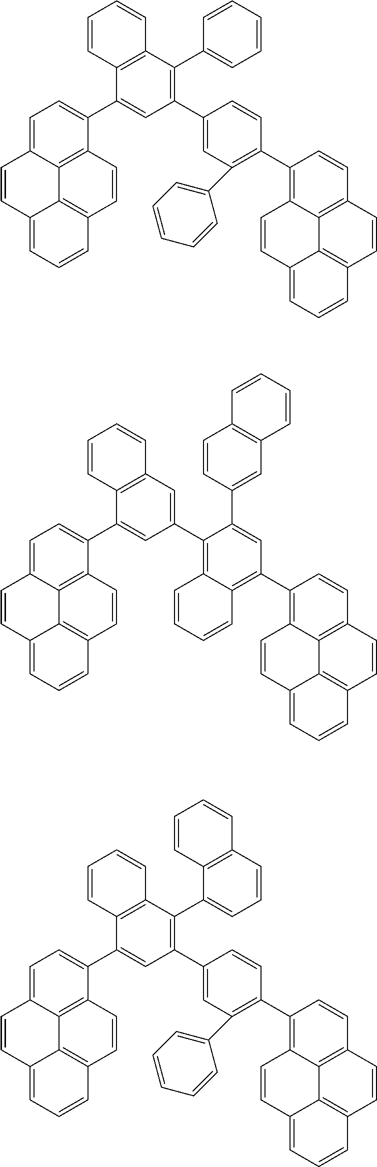

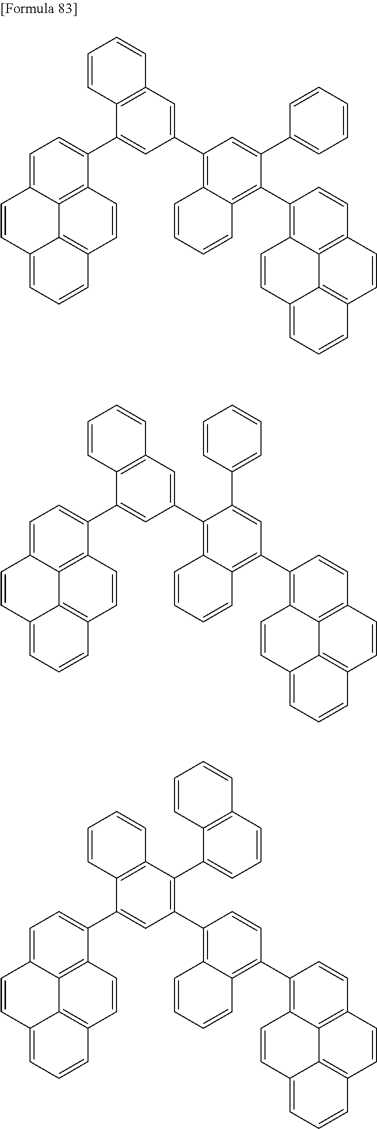

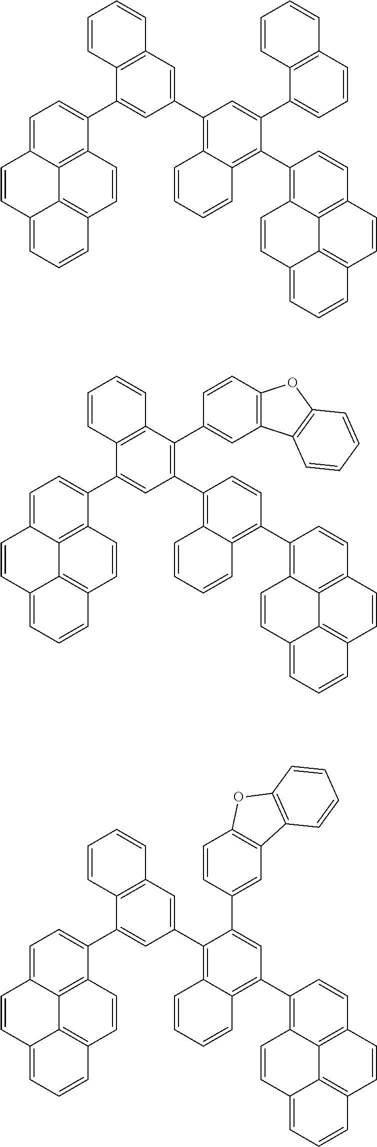

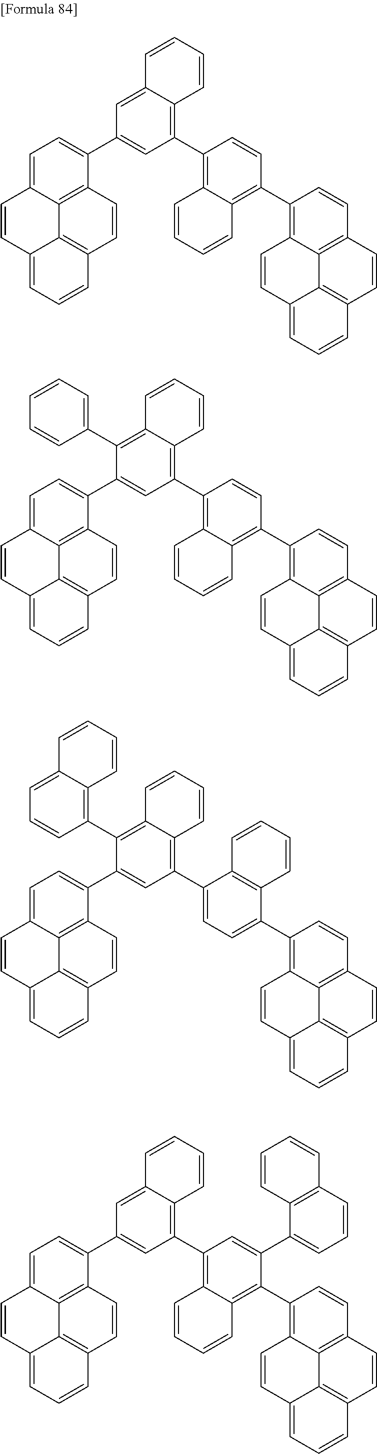

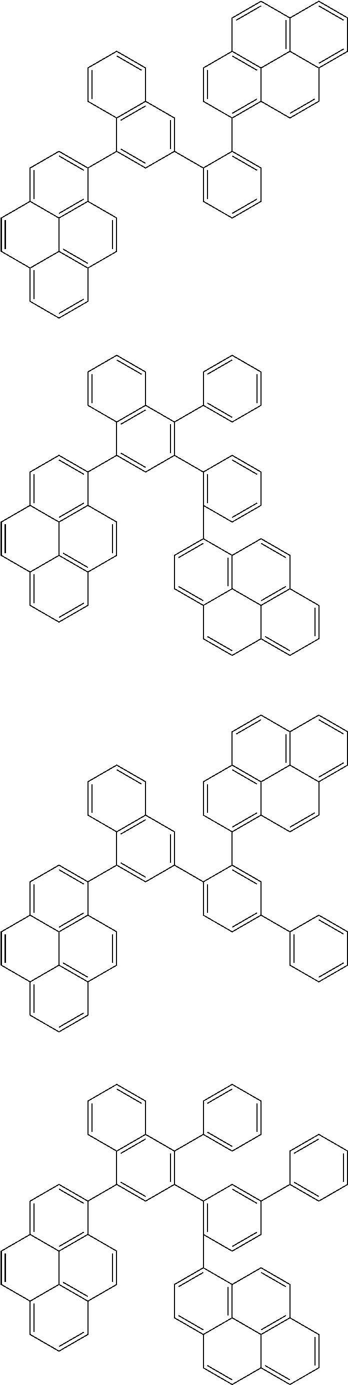

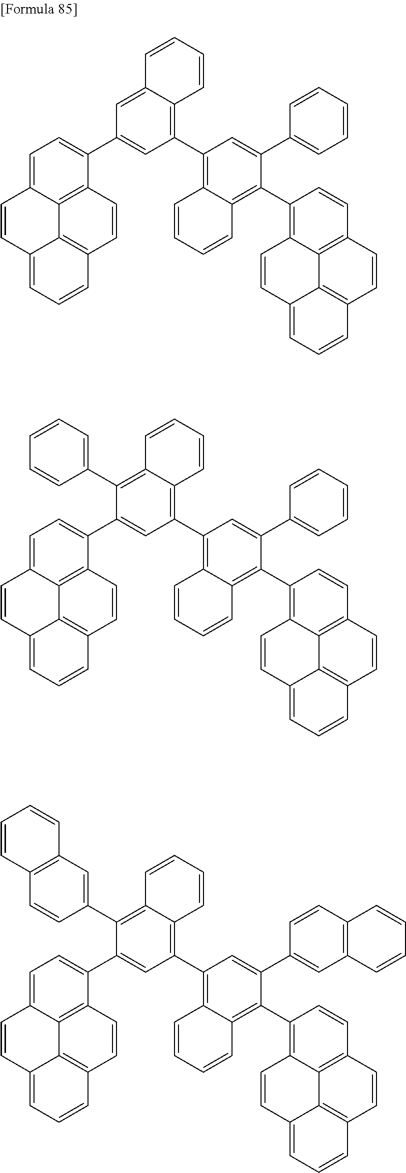

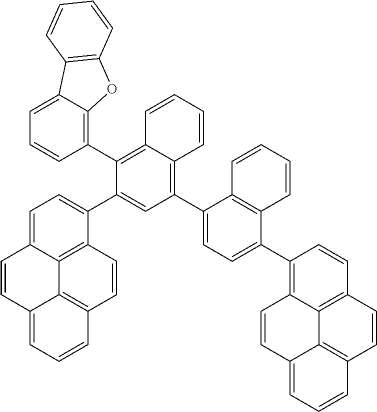 [SMILES: c1cc2ccc3ccc(-c4cc(-c5ccc(-c6ccc7ccc8cccc9ccc6c7c89)c6ccccc56)c5ccccc5c4-c4cccc5c4oc4ccccc45)c4ccc(c1)c2c34]

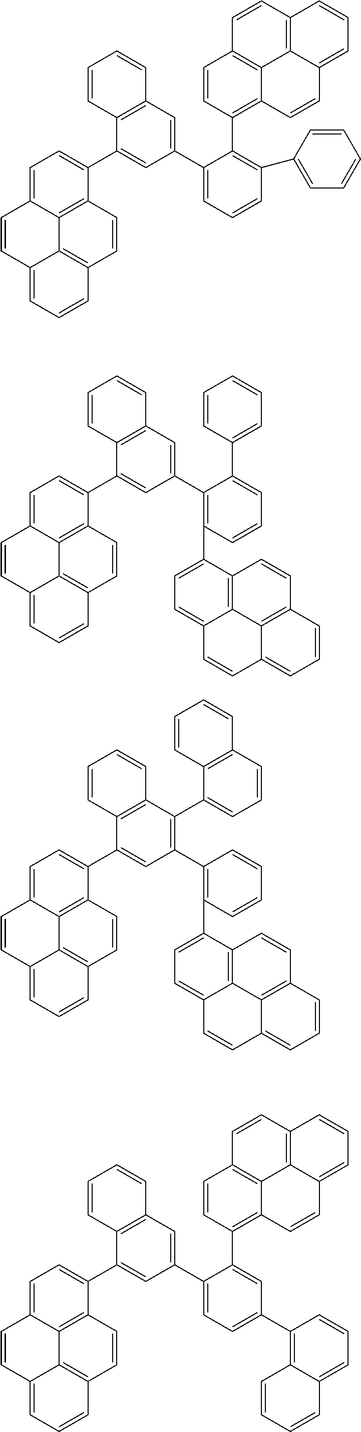

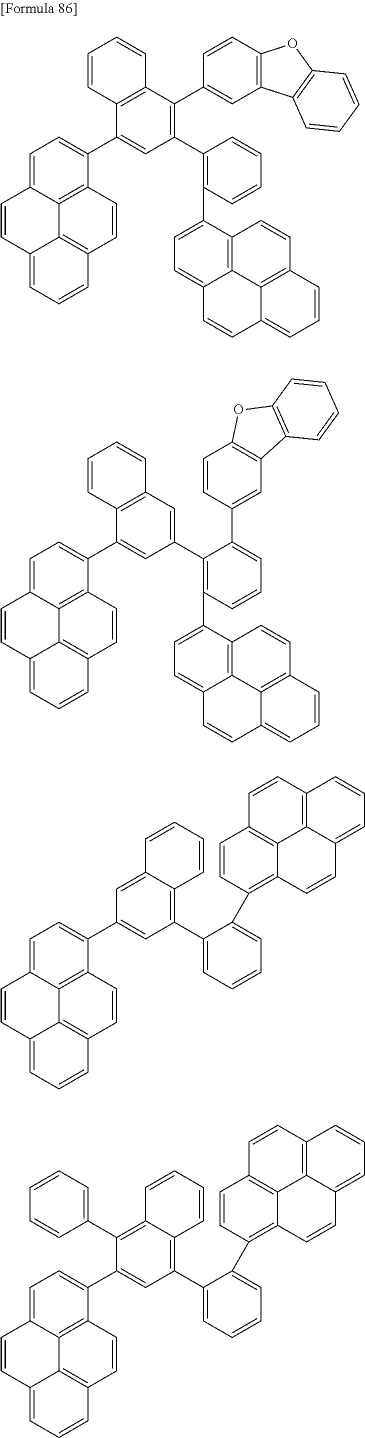

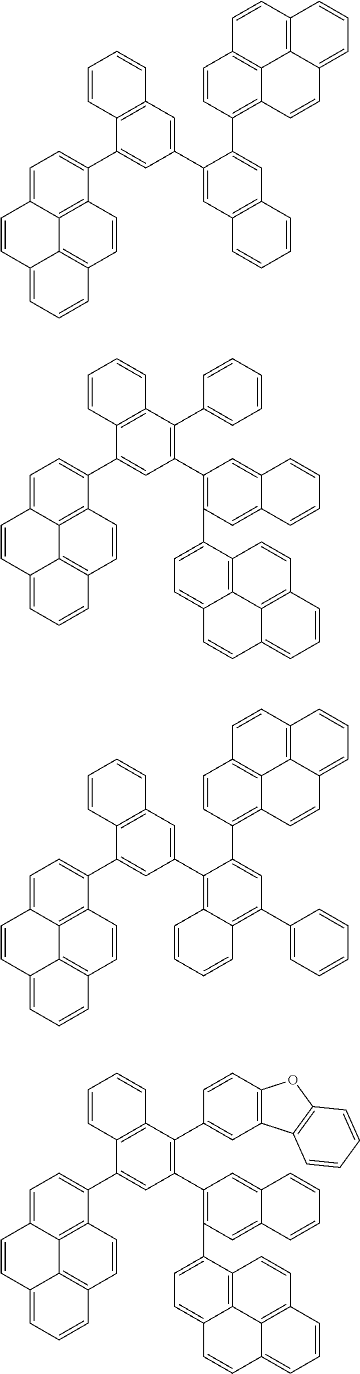

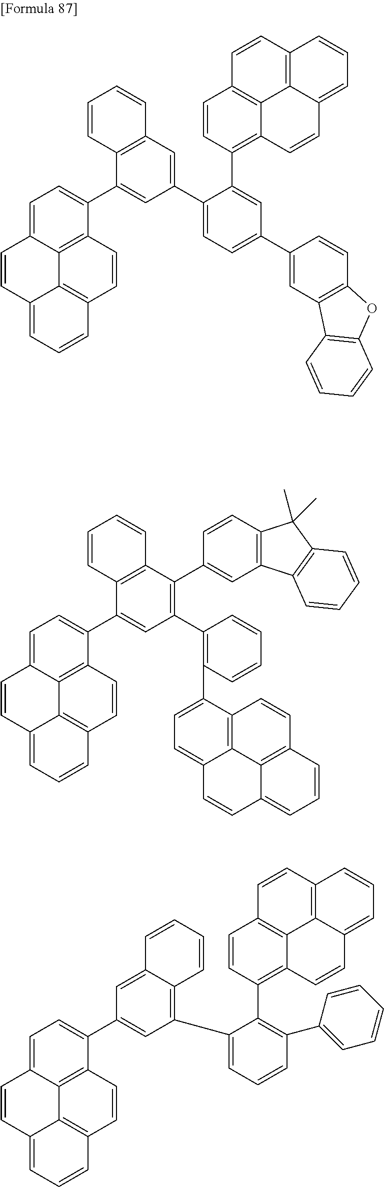

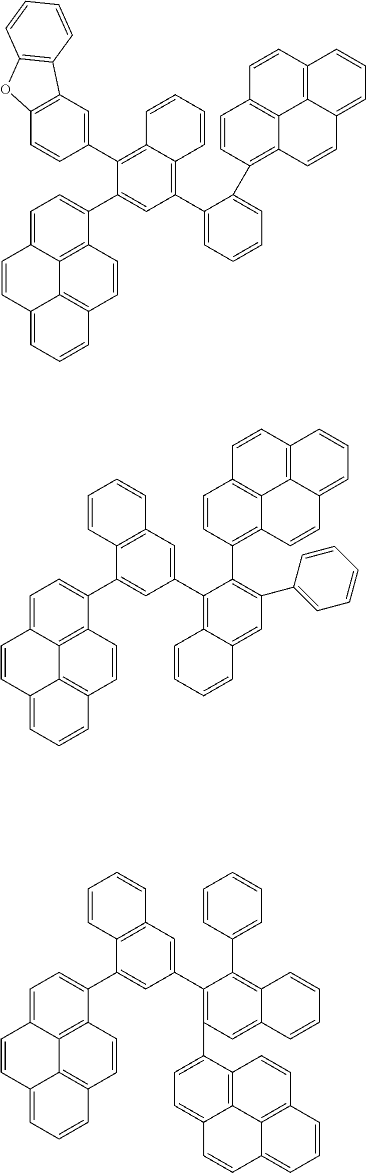

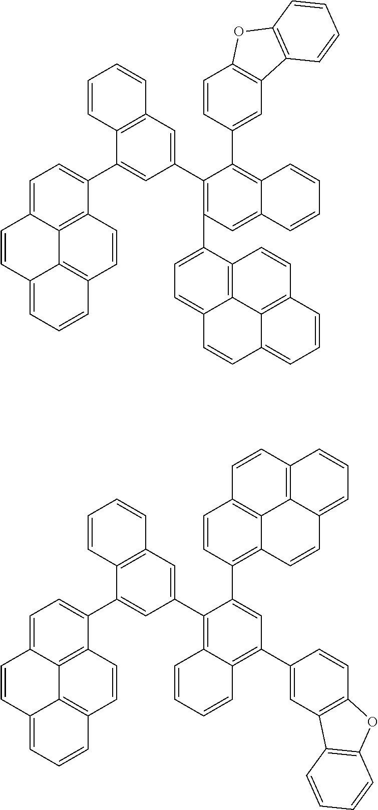 [SMILES: c1ccc2c(-c3ccc4ccc5cccc6ccc3c4c56)cc(-c3c(-c4ccc5ccc6cccc7ccc4c5c67)cc4ccccc4c3-c3ccc4oc5ccccc5c4c3)cc2c1 ; c1ccc2c(-c3ccc4ccc5cccc6ccc3c4c56)cc(-c3c(-c4ccc5ccc6cccc7ccc4c5c67)cc(-c4ccc5oc6ccccc6c5c4)c4ccccc34)cc2c1]

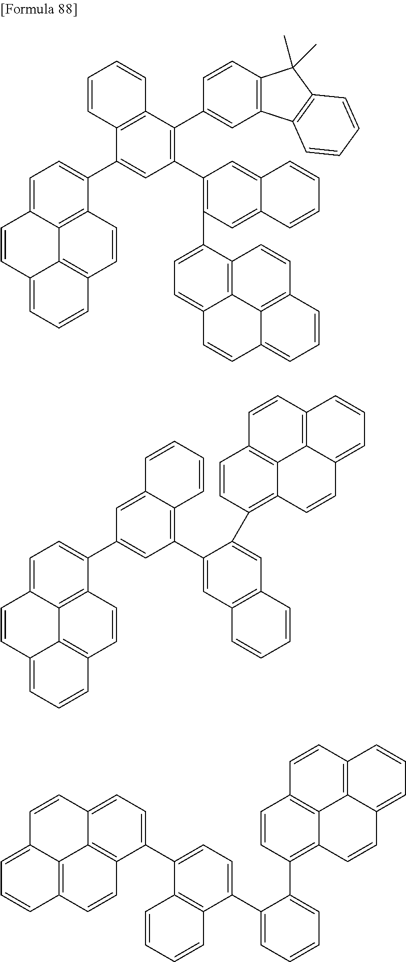

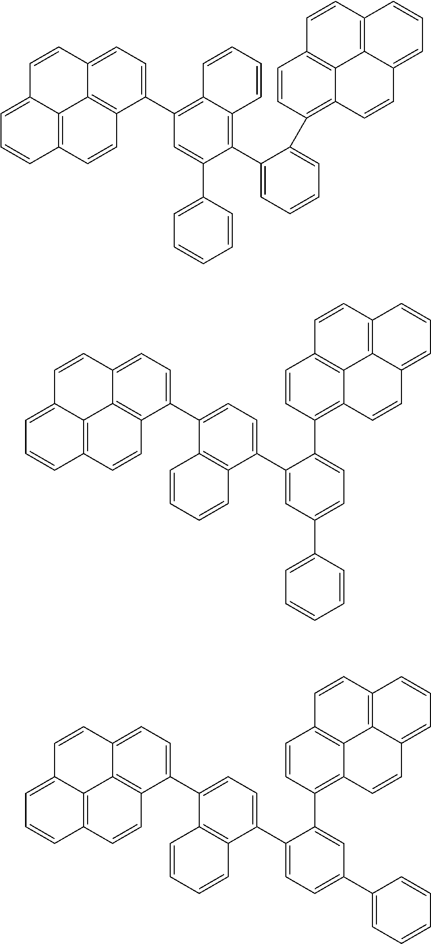

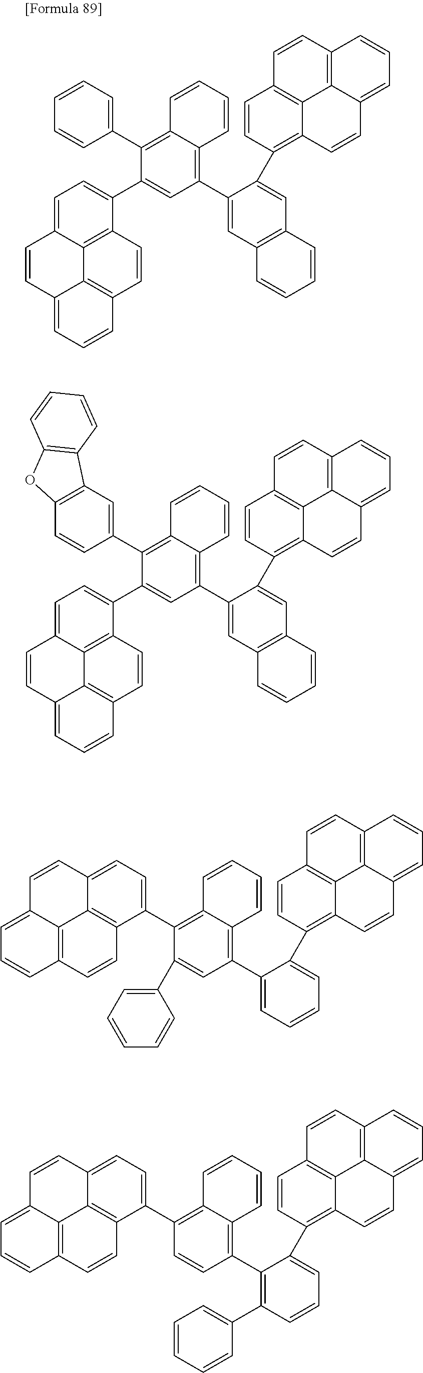

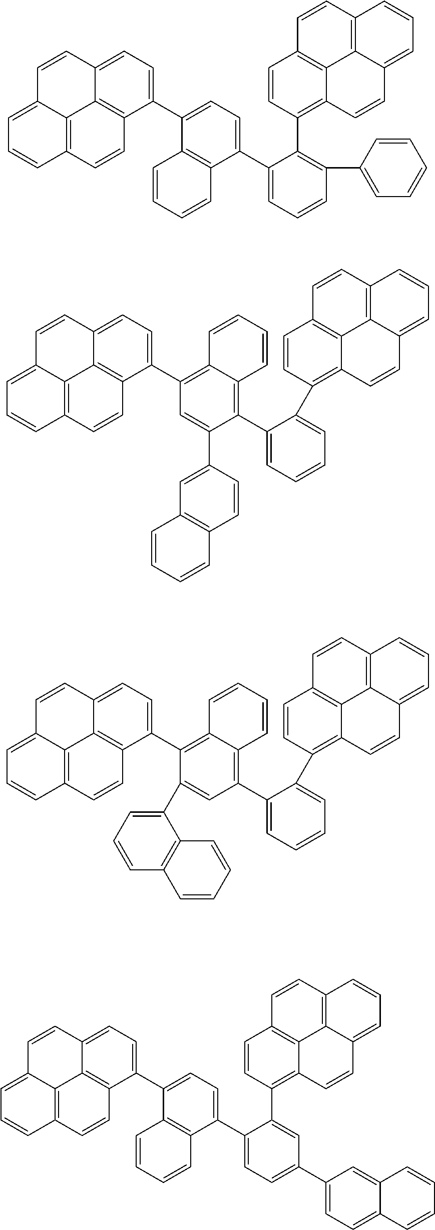

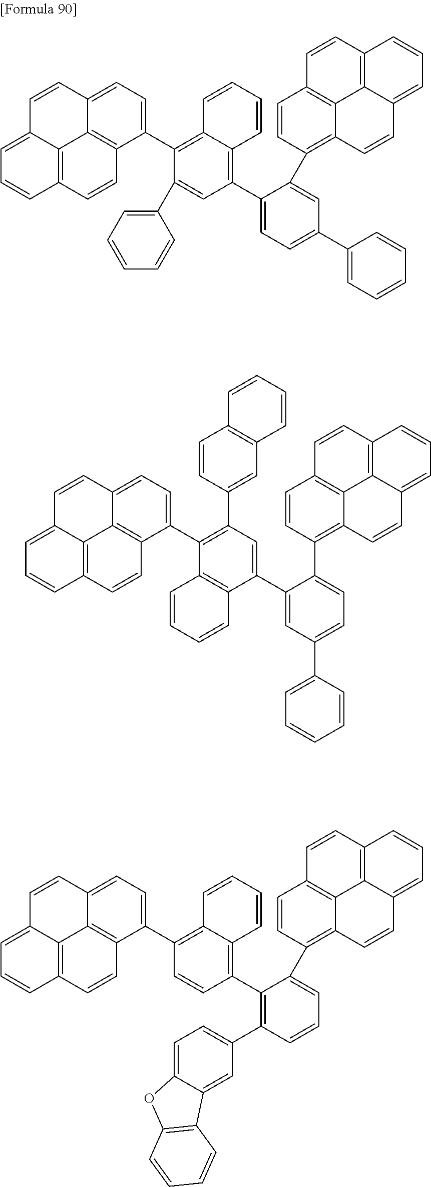

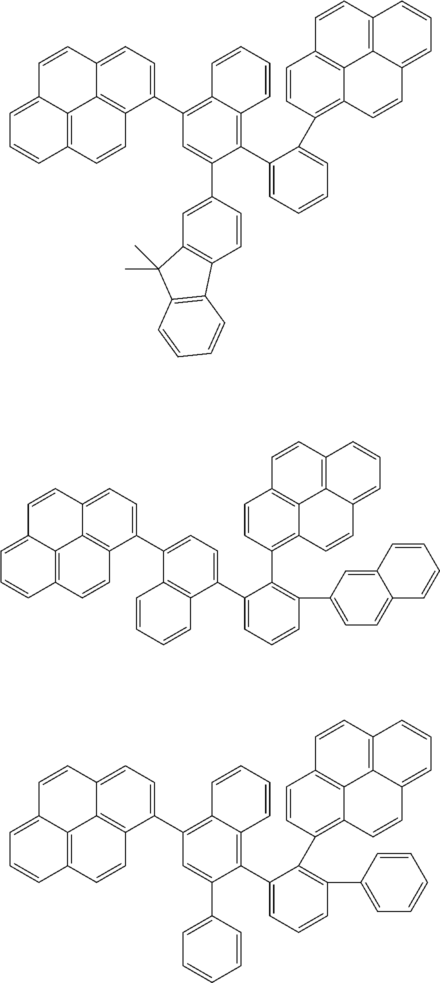

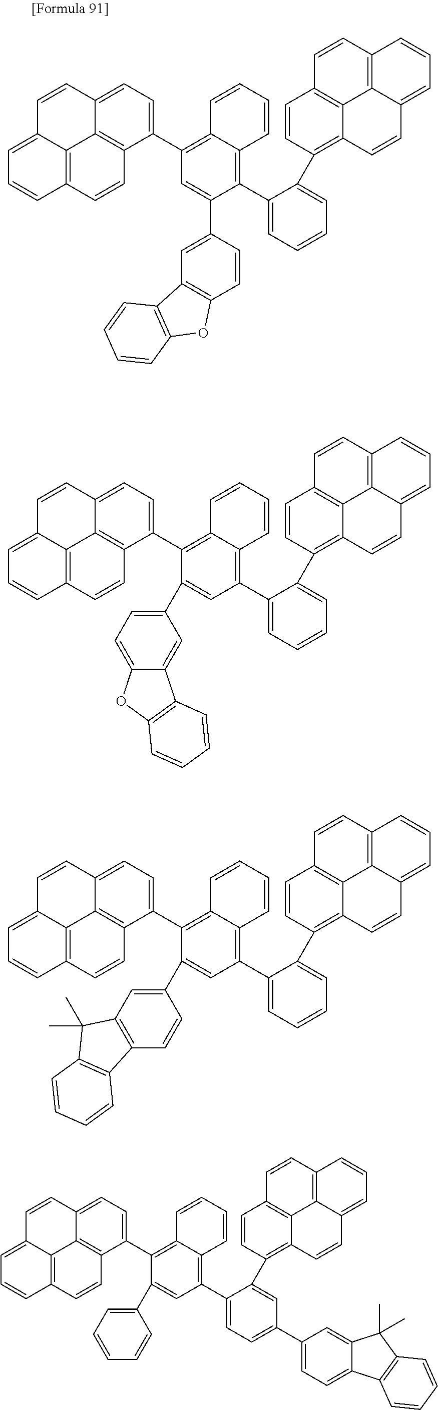

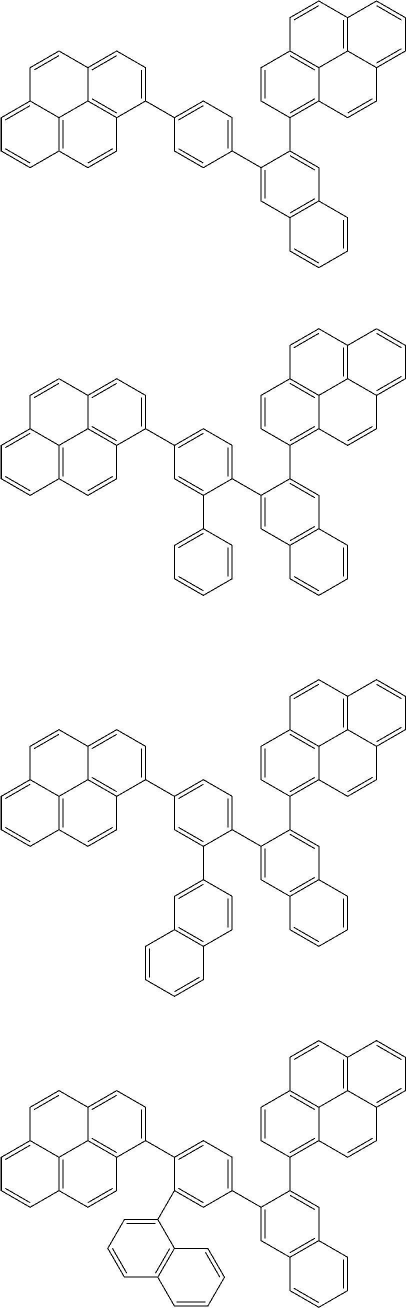

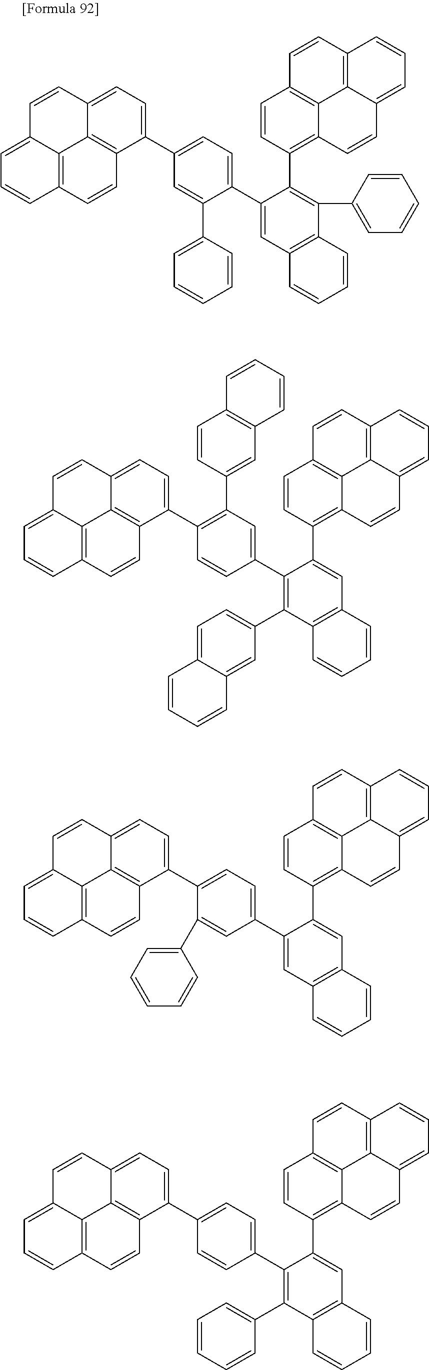

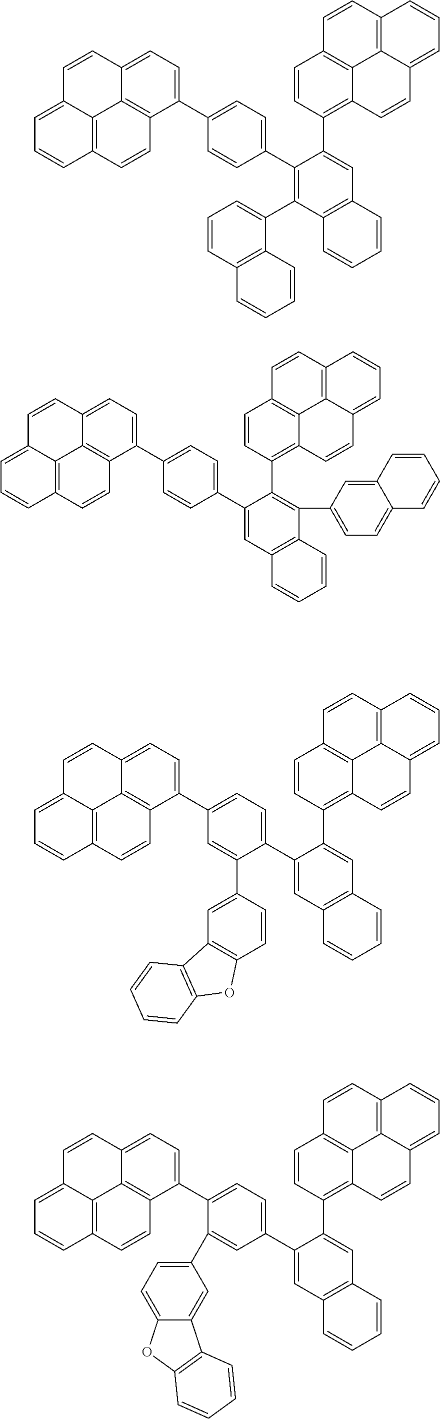

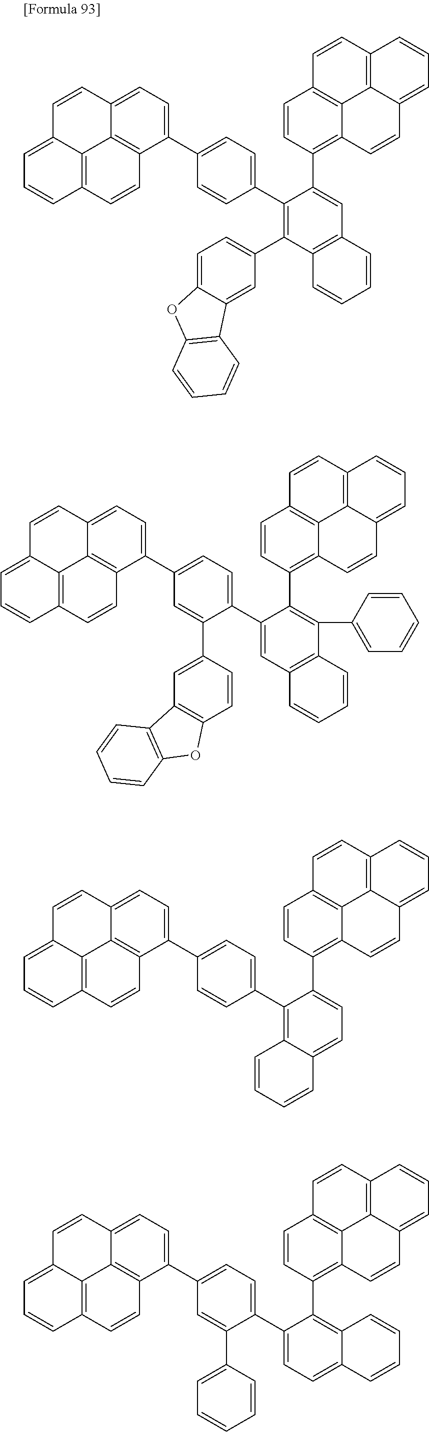

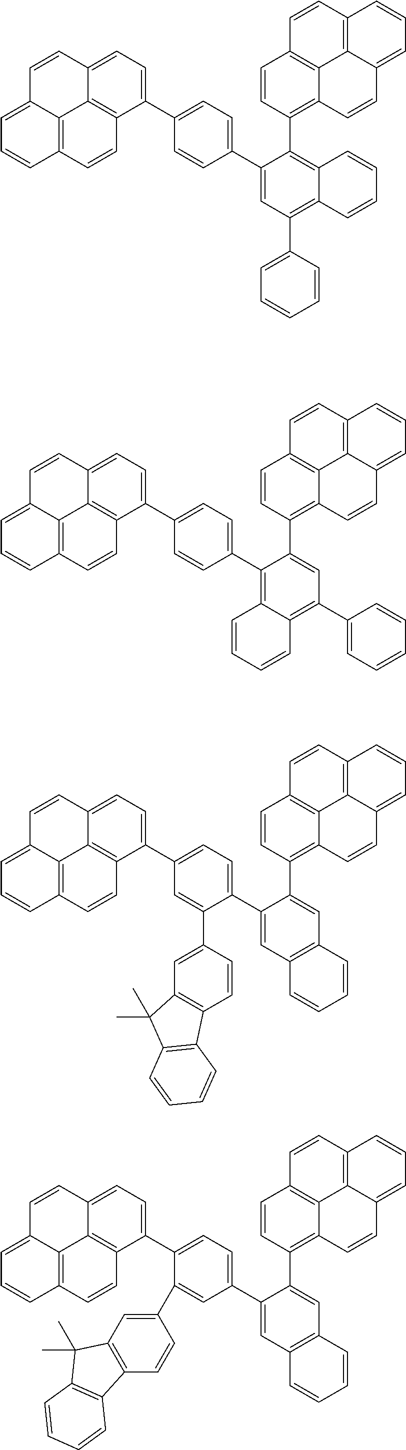

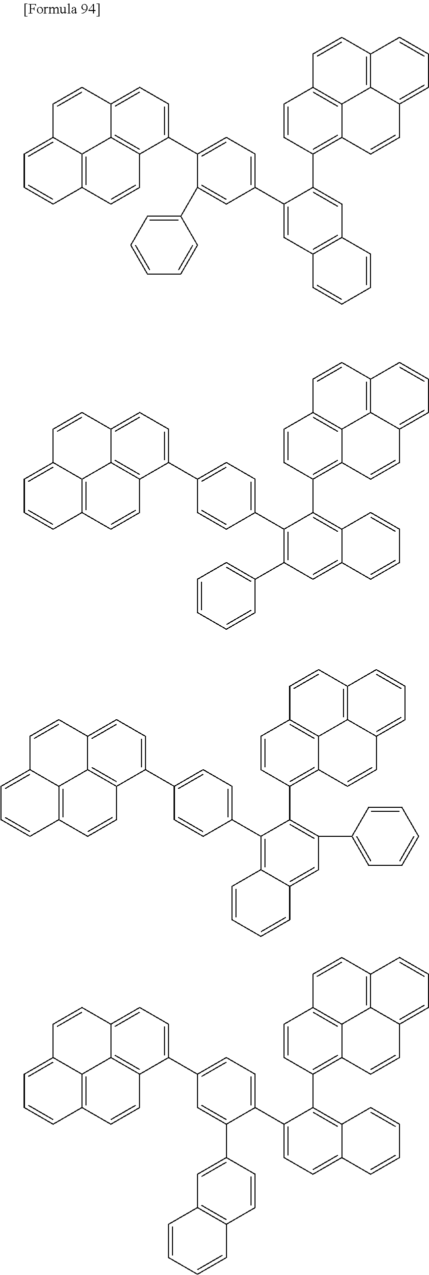

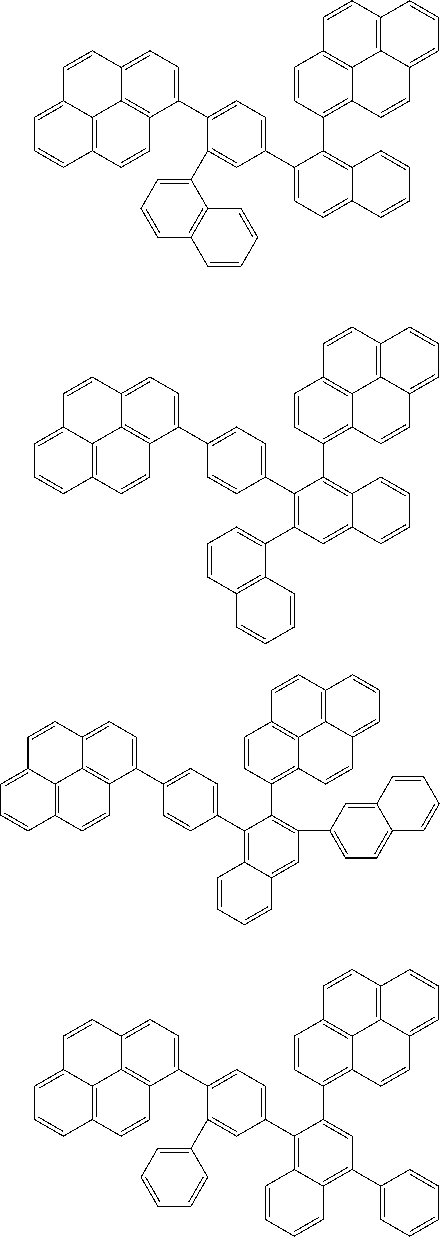

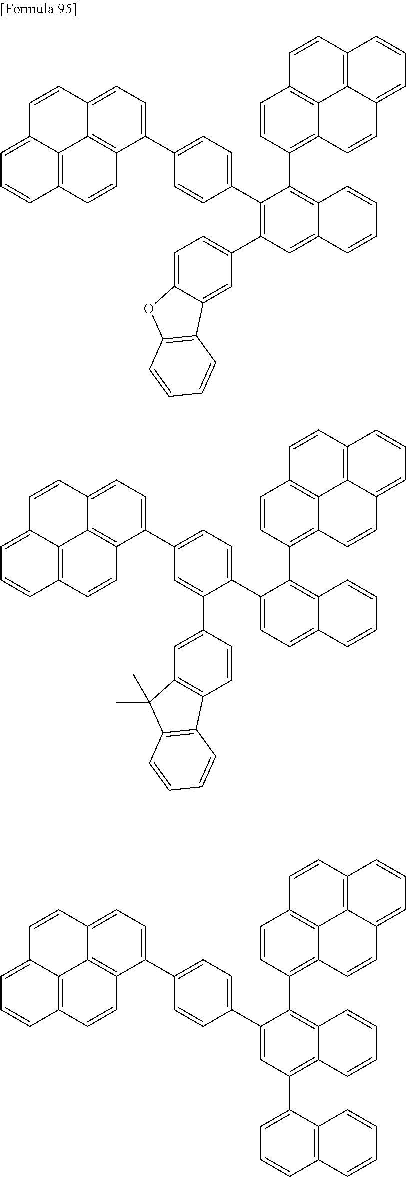

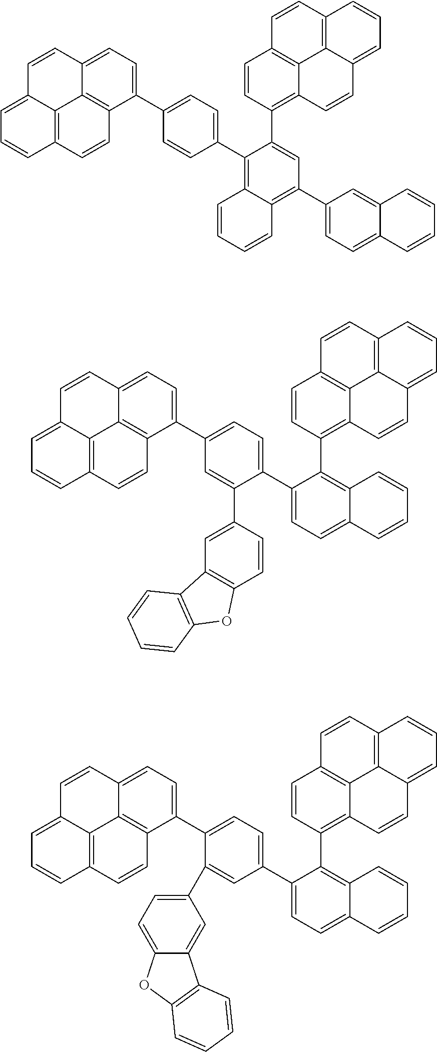

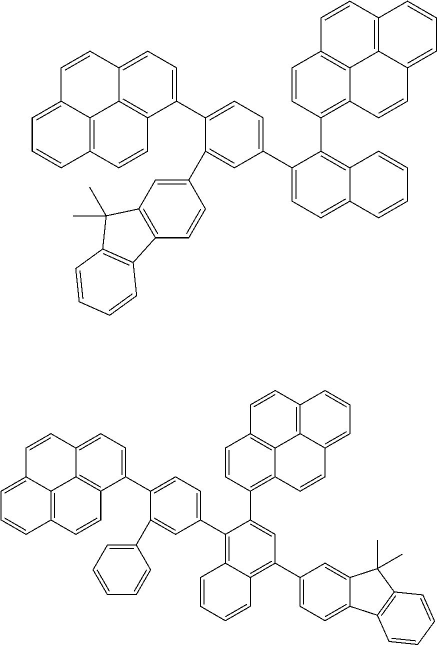

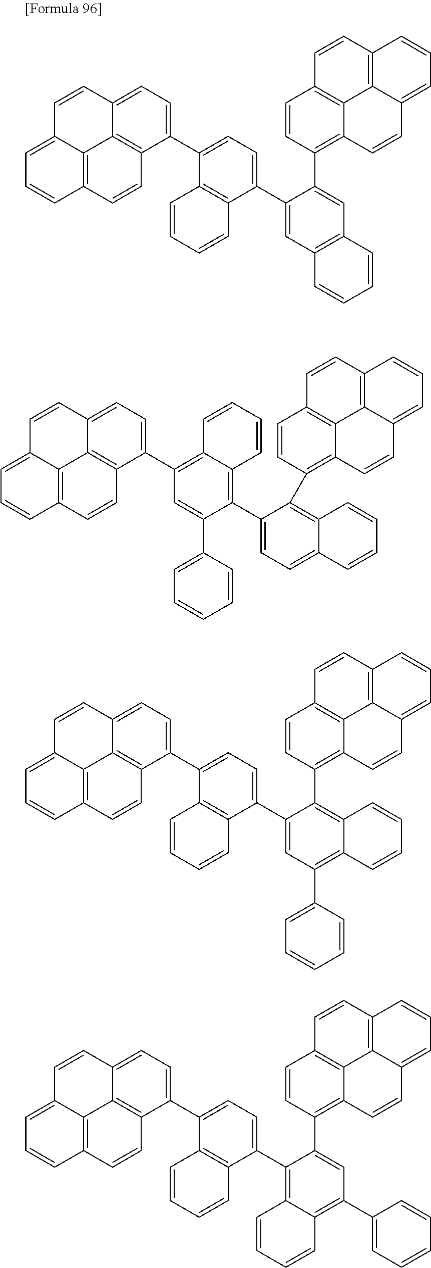

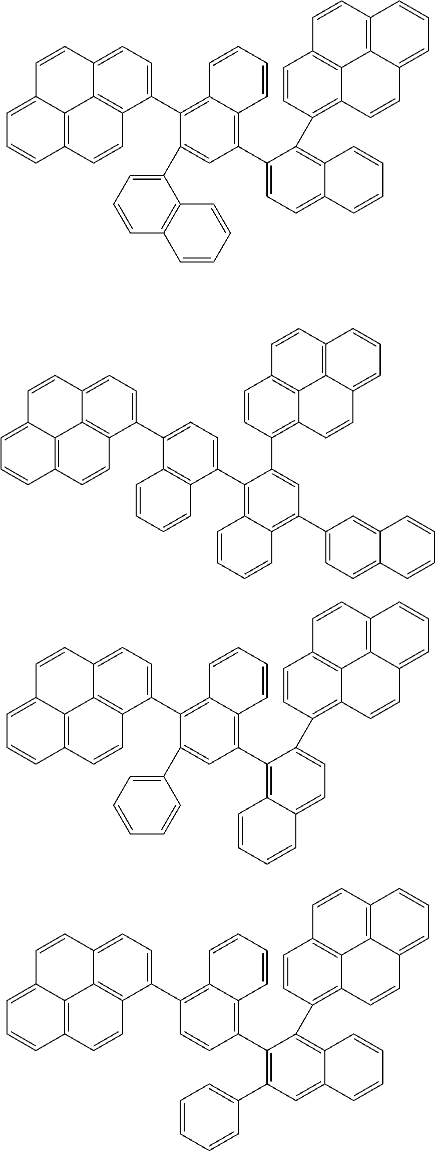

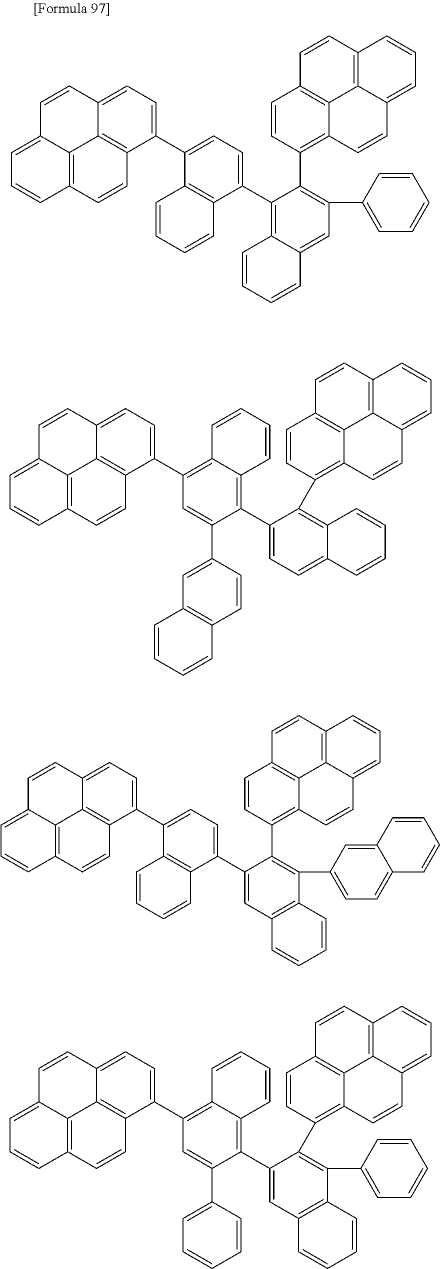

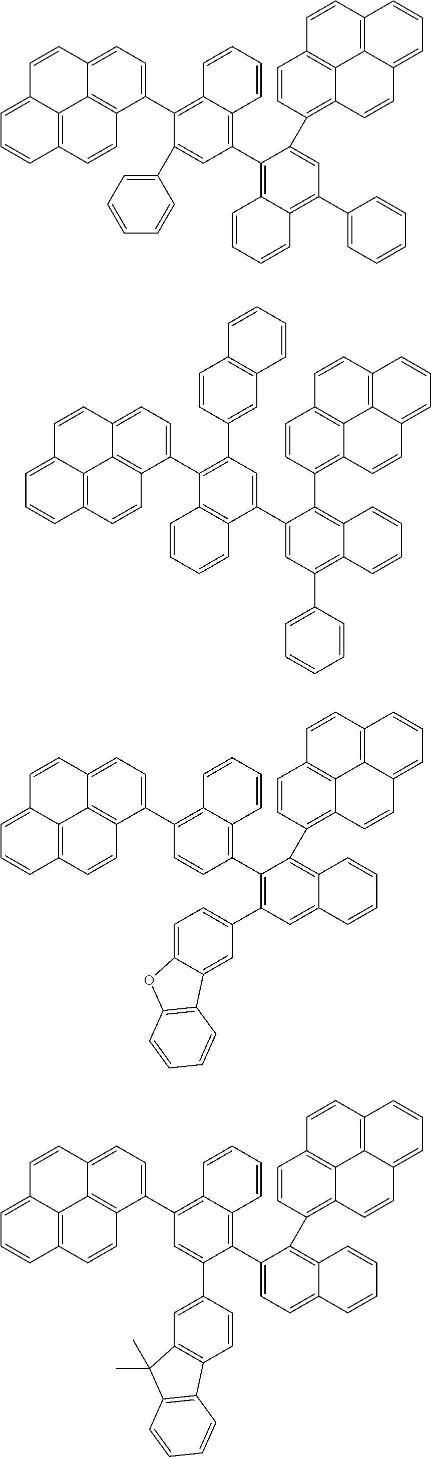

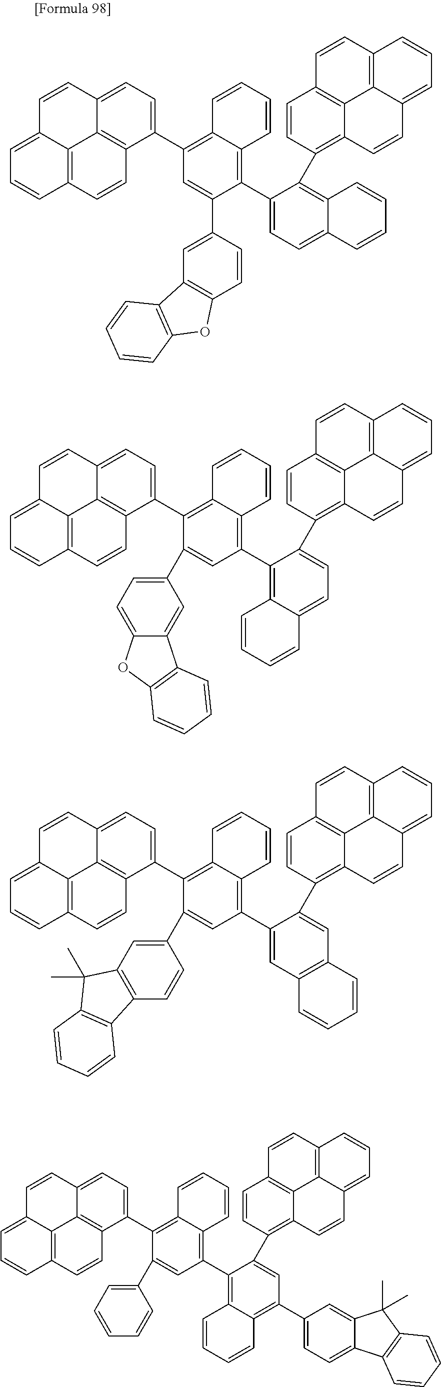

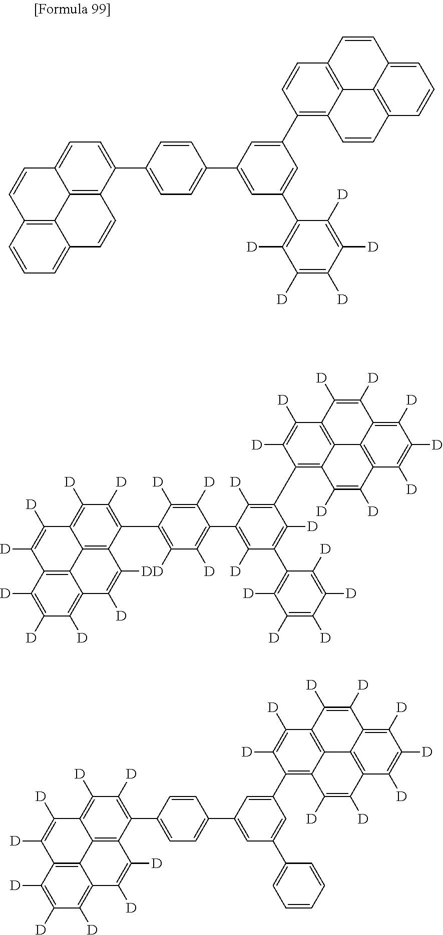

### Third Exemplary Embodiment

### Compound

The compound according to the third exemplary embodiment is a compound represented by the following formula (1), (2), or (3).

The compound according to the third exemplary embodiment (the compound represented by the following formula (1), (2), or (3)) is an embodiment of the first compound (the compound that has at least one group represented by the formula (11) and is represented by the formula (1A)).

The compound according to the third exemplary embodiment is a compound in which R₁₀₁ in the formula (1A) of the first compound is bonded to the formula (11) via a single bond.

A compound represented by the following formula (1), (2), or (3) is one example of the compound according to the first exemplary embodiment (a compound represented by the formula (12X)).

The compound according to the third exemplary embodiment, in which the linker linking two 1-pyrenyl groups in the following formula (1), (2), or (3) has an asymmetric structure (the asymmetric structure 4), has an asymmetric structure as a whole.

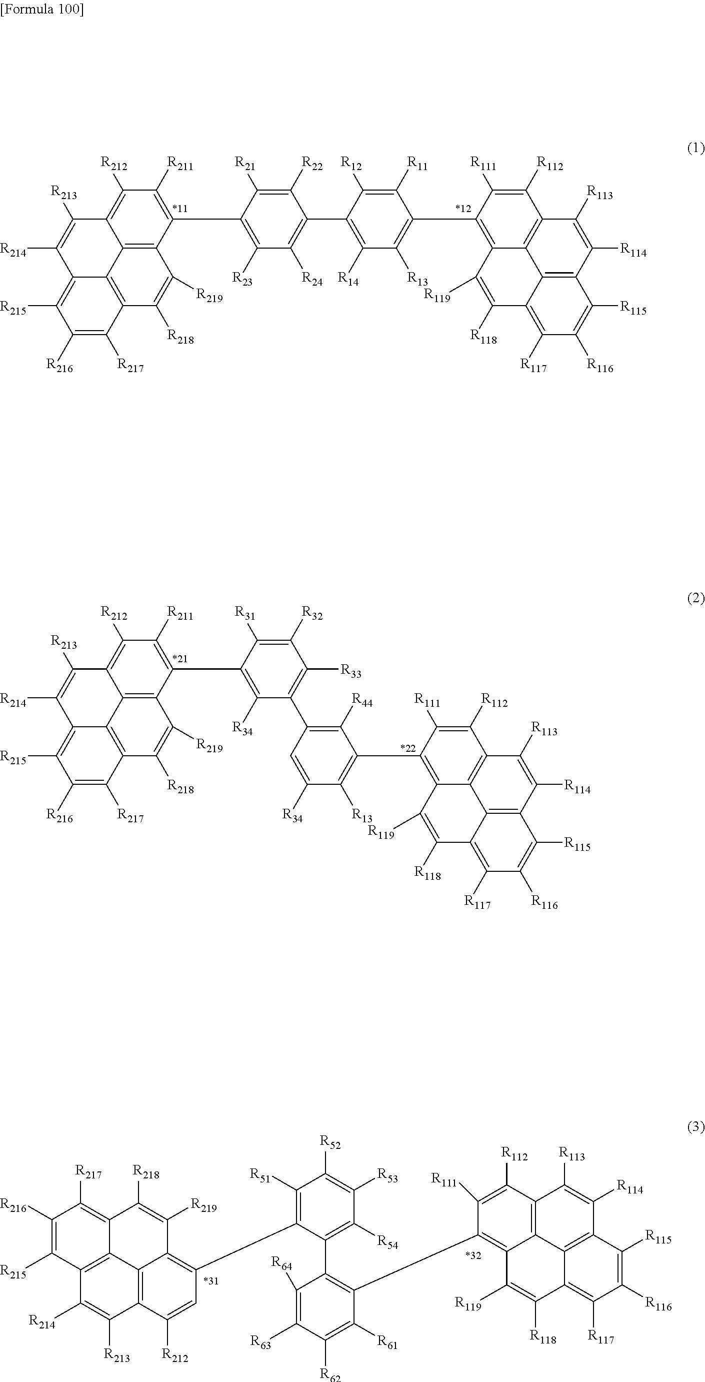

In the formulae (1) to (3),
R₁₁₁ to R₁₁₉ and R₂₁₁ to R₂₁₉ each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms,
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms.

In the formula (1):
at least one combination of a combination of R₁₁ and R₁₂, a combination of R₁₃ and R₁₄, a combination of R₂₁ and R₂₂, or a combination of R₂₃ and R₂₄ are mutually bonded to form a substituted or unsubstituted monocyclic ring, are mutually bonded to form a substituted or unsubstituted fused ring, or are not mutually bonded,
R₁₁, R₁₃, R₂₁, and R₂₃ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4),
R₁₂, R₁₄, R₂₂, and R₂₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4), and
at least one of R₁₁ to R₁₄ or R₂₁ to R₂₄ is not a hydrogen atom.

In the formulae (2) to (3),
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring, are mutually bonded to form a substituted or unsubstituted fused ring, or are not mutually bonded,
R₃₁ to R₃₄, R₄₁ to R₄₄, R₅₁ to R₅₄, and R₆₁ to R₆₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted spirobifluorenyl group, or a group represented by the following formula (4),
at least one of R₃₁ to R₃₄ or R₄₁ to R₄₄ is not a hydrogen atom, and
at least one of R₅₁ to R₅₄ or R₆₁ to R₆₄ is not a hydrogen atom.

In a compound represented by the formula (1), a combination of R₁₁ and R₁₃ and a combination of R₂₁ and R₂₃ are different combinations, or a combination of R₁₂ and R₁₄ and a combination of R₂₂ and R₂₄ are different combinations.

In a compound represented by the formula (2), a combination of R₃₁ and R₄₁ is different from at least one combination of a combination of R₃₂ and R₄₂, a combination of R₃₃ and R₄₃, or a combination of R₃₄ and R₄₄.

In a compound represented by the formula (3), a combination of R₅₁ and R₆₁ is different from at least one combination of a combination of R₅₂ and R₆₂, a combination of R₅₃ and R₆₃, or a combination of R₅₄ and R₆₄.

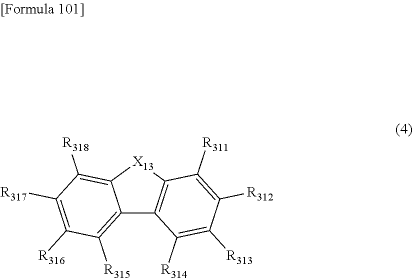

In the formula (4):
X₁₃ represents an oxygen atom, a sulfur atom, or NR₃₁₉,
at least one combination of adjacent two or more of R₃₁₁ to R₃₁₈ are mutually bonded to form a substituted or unsubstituted monocyclic ring, are mutually bonded to form a substituted or unsubstituted fused ring, or are not mutually bonded,
R₃₁₁ to R₃₁₉ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or substituted or unsubstituted 9,9-diphenylfluorenyl group, and
any one of R₃₁₁ to R₃₁₉ that do not form the substituted or unsubstituted monocyclic ring and do not form the substituted or unsubstituted fused ring is a single bond bonded to at least one of R₁₁ to R₁₄, R₂₁ to R₂₄, R₃₁ to R₃₄, R₄₁ to R₄₄, R₅₁ to R₅₄, Or R₆₁ to R₆₄.

In the formulae (1) to (3), a substituent for “substituted or unsubstituted” in R₁₁ to R₁₄, R₂₁ to R₂₄, R₃₁ to R₃₄, R₄₁ to R₄₄, R₅₁ to R₅₄, R₆₁ to R₆₄, R₁₁₁ to R₁₁₉, R₂₁₁ to R₂₁₉, and R₃₁₁ to R₃₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

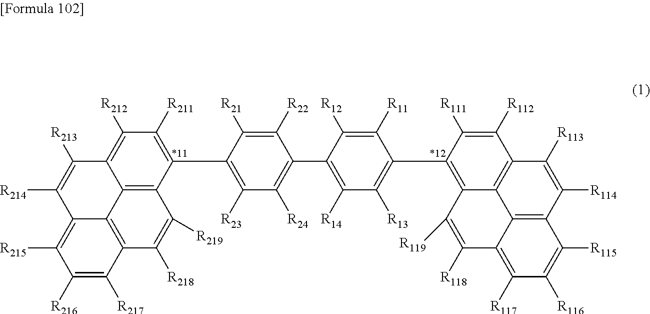

In the formula (1), the phenylene group with R₁₁ to R₁₄ corresponds to L₁ in the formula (12X) of the first exemplary embodiment, and the phenylene group with R₂₁ to R₂₄ corresponds to L₂ in the formula (12X) of the first exemplary embodiment.

In the formula (1), therefore, that “the linker linking two 1-pyrenyl groups has an asymmetric structure” refers to the asymmetric structure 4 in which the bonding position of the phenylene group (the phenylene group with R₁₁ to R₁₄) of L₁ and the bonding position of the phenylene group (the phenylene group with R₂₁ to R₃₄) of L₂ are para positions and are mutually the same, but the structure of L₁ (the phenylene group with R₁₁ to R₁₄) and the structure of L₂ (the phenylene group with R₂₁ to R₃₄) are mutually different including substituents.

In the formula (1), one embodiment in which the linker linking two 1-pyrenyl groups has an asymmetric structure is exemplified by an embodiment in which “a combination of R₁₁ and R₁₃ and a combination of R₂₁ and R₂₃ are different combinations, or a combination of R₁₂ and R₁₄ and a combination of R₂₂ and R₂₄ are different combinations”.

In the formula (1), “a combination of R₁₁ and R₁₃ and a combination of R₂₁ and R₂₃ are different combinations” is described below as an example. The following formulae (1a), (1b), and (1c) each represent a partial structure of the formula (1).

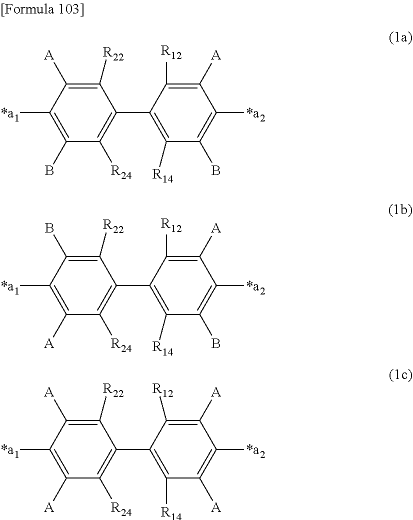

In the formulae (1a), (1b), and (1c):
R₁₂, R₁₄, R₂₂, and R₂₄ represent the same as R₁₂, R₁₄, R₂₂, and R₂₄, respectively, in the formula (1),
*a₁ represents the position of bonding to *11 in the formula (1), and
*a₂ represents the position of bonding to *12 in the formula (1).

For example, when R₁₁ represents “A” and R₁₃ represents “B”, that a combination of R₁₁ and R₁₃ and a combination of R₂₁ and R₂₃ are the same combination refers to that R₂₁ represents “A” and R₂₃ represents “B” (the formula (1a)), or R₂₁ represents “B” and R₂₃ represents “A” (the formula (1b)).

However, “A” is different from “B”.

When both R₁₁ and R₁₃ represent “A”, that a combination of R₁₁ and R₁₃ and a combination of R₂₁ and R₂₃ are the same combination refers to that both R₂₁ and R₂₃ represent “A” (the formula (1c)).

Thus, when a combination of R₁₁ and R₁₃ and a combination of R₂₁ and R₂₃ are different combinations, for example, R₁₁ represents “A”, and R₁₃ represents “B”, and both R₂₁ and R₂₃ represent “A”, or both R₂₁ and R₂₃ represent “B”, or at least one of R₂₁ or R₂₃ represents “C” that is different from “A” and “B”, or alternatively, both R₁₁ and R₁₃ represent “A”, and at least one of R₂₁ or R₂₃ represents “B”, or at least one of R₂₁ or R₂₃ represents “C” that is different from “A”.

The same applies to that “a combination of R₁₂ and R₁₄ and a combination of R₂₂ and R₂₄ are different combinations” in the formula (1).

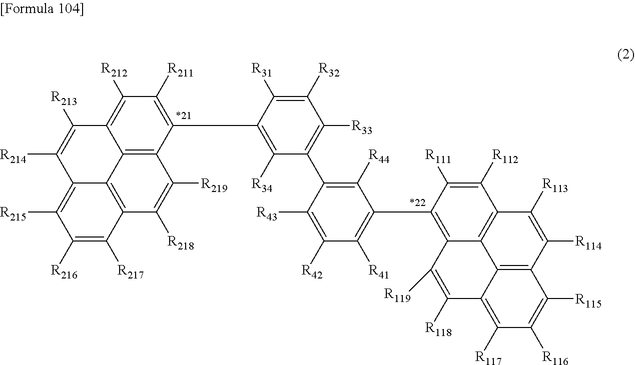

In the formula (2), the phenylene group with R₃₁ to R₃₄ corresponds to L₁ in the formula (12X) of the first exemplary embodiment, and the phenylene group with R₄₁ to R₄₄ corresponds to L₂ in the formula (12X) of the first exemplary embodiment.

In the formula (2), therefore, that “the linker linking two 1-pyrenyl groups has an asymmetric structure” refers to the asymmetric structure 4 in which the bonding position of the phenylene group (the phenylene group with R₃₁ to R₃₄) of L₁ and the bonding position of the phenylene group (the phenylene group with R₄₁ to R₄₄) of L₂ are meta positions and are mutually the same, but the structure of L₁ (the phenylene group with R₃₁ to R₃₄) and the structure of L₂ (the phenylene group with R₄₁ to R₄₄) are mutually different including substituents.

More specifically, that “the linker linking two 1-pyrenyl groups has an asymmetric structure” means that R₃₁ is different from R₄₁, R₃₂ is different from R₄₂, R₃₃ is different from R₄₃, or R₃₄ is different from R₄₄.

In the formula (2), one embodiment in which the linker linking two 1-pyrenyl groups has an asymmetric structure is exemplified by an embodiment in which “a combination of R₃₁ and R₄₁ is different from at least one combination of a combination of R₃₂ and R₄₂, a combination of R₃₃ and R₄₃, or a combination of R₃₄ and R₄₄”.

“A combination of R₃₁ and R₄₁ is different from at least one combination of a combination of R₃₂ and R₄₂, a combination of R₃₃ and R₄₃, or a combination of R₃₄ and R₄₄” in the formula (2) is described below with respect to an example in which “a combination of R₃₁ and R₄₁ is different from a combination of R₃₂ and R₄₂”. The following formulae (2a), (2b), and (2c) represent a partial structure of the formula (2).

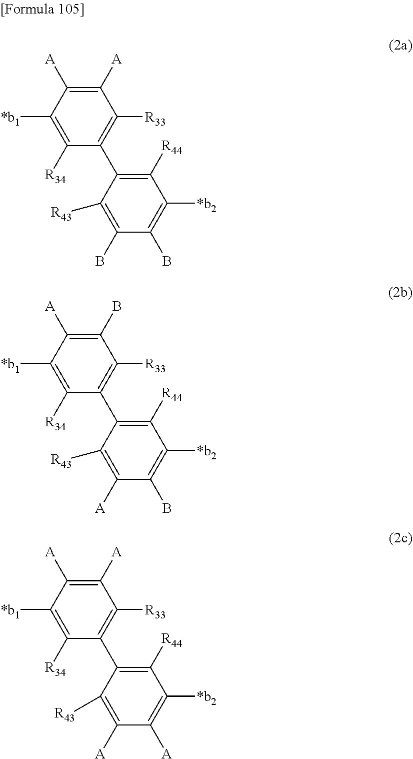

In the formulae (2a), (2b), and (2c), R₃₃, R₃₄, R₄₃, and R₄₄ represent the same as R₃₃, R₃₄, R₄₃, and R₄₄, respectively, in the formula (2).
*b₁ represents the position of bonding to *21 in the formula (2).
*b₂ represents the position of bonding to *22 in the formula (2).

For example, when R₃₁ represents “A”, and R₄₁ represents “B”, that a combination of R₃₁ and R₄₁ and a combination of R₃₂ and R₄₂ are the same combination refers to that R₃₂ represents “A”, and R₄₂ represents “B” (the formula (2a)), or R₃₂ represents “B”, and R₄₂ represents “A” (the formula (2b).

However, “A” is different from “B”.

When both R₃₁ and R₄₁ represent “A”, that a combination of R₃₁ and R₄₁ and a combination of R₃₂ and R₄₂ are the same combination refers to that both R₃₂ and R₄₂ represent “A” (the formula (2c)).

More specifically, when a combination of R₃₁ and R₄₁ and a combination of R₃₂ and R₄₂ are different combinations, for example, R₃₁ represents “A”, R₄₁ represents “B”, and both R₃₂ and R₄₂ represent “A”, or both R₃₂ and R₄₂ represent “B”, or at least one of R₃₂ or R₄₂ represents “C” that is different from “A” and “B”, or alternatively, both R₃₁ and R₄₁ represent “A”, and at least one of R₃₂ or R₄₂ represents “B”, or at least one of R₃₂ or R₄₂ represents “C” that is different from “A”.

The same applies to “a combination of R₃₁ and R₄₁ and a combination of R₃₃ and R₄₃ are different combinations” and “a combination of R₃₁ and R₄₁ and a combination of R₃₄ and R₄₄ are different combinations” in the formula (2).

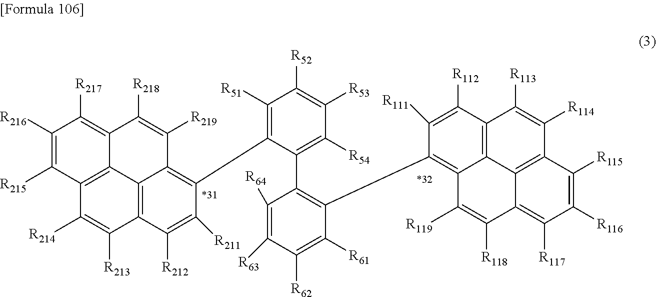

In the formula (3), the phenylene group with R₅₁ to R₅₄ corresponds to L₁ in the formula (12X) of the first exemplary embodiment, and the phenylene group with R₆₁ to R₆₄ corresponds to L₂ in the formula (12X) of the first exemplary embodiment.

In the formula (3), therefore, that “the linker linking two 1-pyrenyl groups has an asymmetric structure” refers to the asymmetric structure 4 in which the bonding position of the phenylene group (the phenylene group with R₅₁ to R₅₄) of L₁ and the bonding position of the phenylene group (the phenylene group with R₆₁ to R₆₄) of L₂ are ortho positions and are mutually the same, but the structure of L₁ (the phenylene group with R₅₁ to R₅₄) and the structure of L₂ (the phenylene group with R₆₁ to R₆₄) are mutually different including substituents.

More specifically, that “the linker linking two 1-pyrenyl groups has an asymmetric structure” means that R₅₁ is different from R₆₁, R₅₂ is different from R₆₂, R₅₃ is different from R₆₃, or R₅₄ is different from R₆₄.

In the formula (2), one embodiment in which the linker linking two 1-pyrenyl groups has an asymmetric structure is exemplified by an embodiment in which “a combination of R₅₁ and R₆₁ is different from at least one combination of a combination of R₅₂ and R₆₂, a combination of R₅₃ and R₆₃, or a combination of R₅₄ and R₆₄”.

“A combination of R₅₁ and R₆₁ is different from at least one combination of a combination of R₅₂ and R₆₂, a combination of R₅₃ and R₆₃, or a combination of R₅₄ and R₆₄” in the formula (3) is described below with respect to an example in which “a combination of R₅₁ and R₆₁ is different from a combination of R₅₂ and R₆₂”. The following formulae (3a), (3b), and (3c) represent a partial structure of the formula (3).

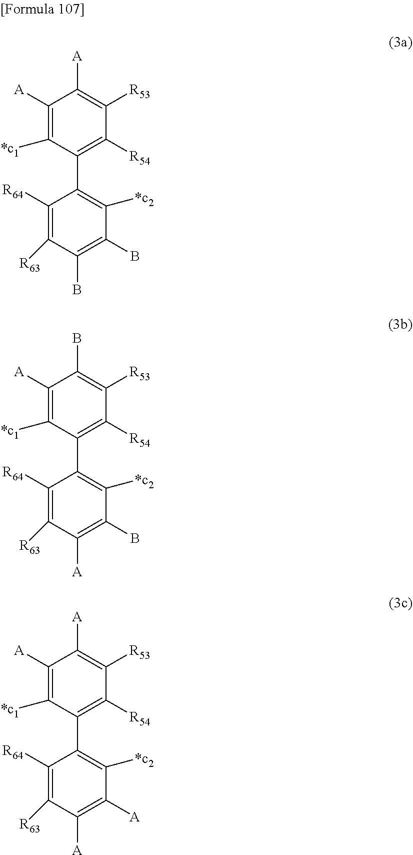

In the formulae (3a), (3b), and (3c),
R₅₃, R₅₄, R₆₃, and R₆₄ represent the same as R₅₃, R₅₄, R₆₃, and R₆₄, respectively, in the formula (3).
*c₁ represents the position of bonding to *31 in the formula (3).
*c₂ represents the position of bonding to *32 in the formula (3).

For example, when R₅₁ represents “A” and R₆₁ represents “B”, that a combination of R₅₁ and R₆₁ and a combination of R₅₂ and R₆₂ are the same combination refers to that R₅₂ represents “A” and R₆₂ represents “B” (the formula (3a)), or R₅₂ represents “B” and R₆₂ represents “A” (the formula (3b)).

However, “A” is different from “B”.

When both R₅₁ and R₆₁ represent “A”, that a combination of R₅₁ and R₆₁ and a combination of R₅₂ and R₆₂ are the same combination refers to that both R₅₂ and R₆₂ represent “A” (the formula (3c)).

Thus, when a combination of R₅₁ and R₆₁ and a combination of R₅₂ and R₆₂ are different combinations refers to, for example, when R₅₁ represents “A” and R₆₁ represents “B”, both R₅₂ and R₆₂ represent “A”, or both R₅₂ and R₆₂ represent “B”, or at least one of R₅₂ or R₆₂ represents “C” that is different from “A” and “B”, when both R₅₁ and R₆₁ represent “A” and at least one of R₅₂ or R₆₂ represents “B”, or when at least one of R₅₂ or R₆₂ represents “C” that is different from “A”.

The same applies to “a combination of R₅₁ and R₆₁ and a combination of R₅₃ and R₆₃ are different combinations” and “a combination of R₅₁ and R₆₁ and a combination of R₅₄ and R₆₄ are different combinations” in the formula (3).

In the compound according to the third exemplary embodiment, R₁₁₁ to R₁₁₉ and R₂₁₁ to R₂₁₉ preferably each independently represent a hydrogen atom, a substituted or unsubstituted alkyl group having 1 to 18 carbon atoms, a substituted or unsubstituted cycloalkyl group having 1 to 18 carbon atoms, a group represented by —Si(Rx)(Ry)(Rz), a substituted or unsubstituted aryl group having 6 to 18 ring carbon atoms, or a substituted or unsubstituted heterocyclic group having 5 to 18 ring atoms. Rx, Ry, and Rz in the —Si(Rx)(Ry)(Rz) preferably each independently represent an unsubstituted alkyl group having 1 to 18 carbon atoms or an unsubstituted aryl group having 6 to 18 ring carbon atoms.

In the compound according to the third exemplary embodiment, R₁₁₁ to R₁₁₉ and R₂₁₁ to R₂₁₉ preferably each independently represent a hydrogen atom, a substituted or unsubstituted alkyl group having 1 to 18 carbon atoms, a substituted or unsubstituted phenyl group, a substituted or unsubstituted naphthyl group, a substituted or unsubstituted phenanthryl group, a substituted or unsubstituted fluorenyl group, a substituted or unsubstituted dibenzofuranyl group, a substituted or unsubstituted dibenzothiophenyl group, a substituted or unsubstituted 9,9-dimethylfluorenyl group, or a substituted or unsubstituted 9,9-diphenylfluorenyl group.

In the compound according to the third exemplary embodiment, R₁₁₁ to R₁₁₉ and R₂₁₁ to R₂₁₉ preferably represent a hydrogen atom.

In the compound according to the third exemplary embodiment, preferably, at least one combination of a combination of R₁₁ and R₁₂, a combination of R₁₃ and R₁₄, a combination of R₂₁ and R₂₂, or a combination of R₂₃ and R₂₄ are not mutually bonded, and at least one combination of adjacent two or more of R₃₁ to R₃₃, R₄₁ to R₄₃, R₅₁ to R₅₄, and R₆₁ to R₆₄ are not mutually bonded.

The compound according to the third exemplary embodiment is preferably represented by the following formula (1-1), (2-1), or (3-1).

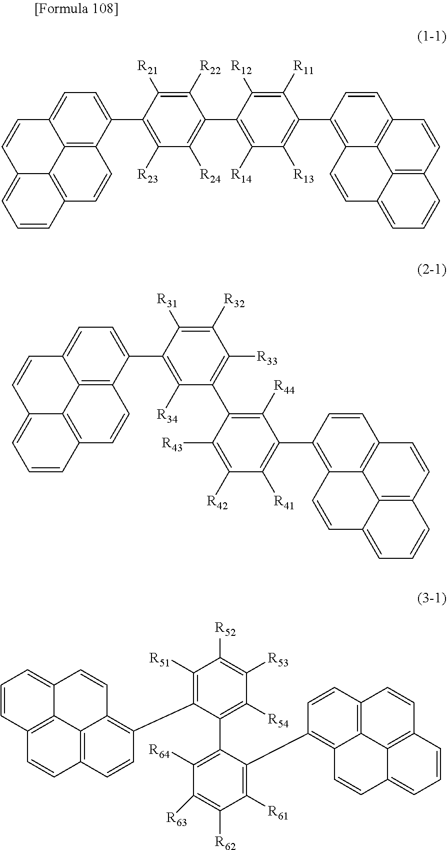

In the formulae (1-1), (2-1), and (3-1), R₁₁ to R₁₄, R₂₁ to R₂₄, R₃₁ to R₃₄, R₄₁ to R₄₄, R₅₁ to R₅₄, and R₆₁ to R₆₄ represent the same as R₁₁ to R₁₄, R₂₁ to R₂₄, R₃₁ to R₃₄, R₄₁ to R₄₄, R₅₁ to R₅₄, and R₆₁ to R₆₄, respectively, in the formulae (1) to (3).

In the compound according to the third exemplary embodiment, R₁₁ to R₁₄, R₂₁ to R₂₄, R₃₁ to R₃₄, R₄₁ to R₄₄, R₅₁ to R₅₄, and R₆₁ to R₆₄ preferably each independently represent a hydrogen atom, a substituted or unsubstituted alkyl group having 1 to 8 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, a substituted or unsubstituted 9,9-dimethylfluorenyl group, a substituted or unsubstituted 9,9-diphenylfluorenyl group, a substituted or unsubstituted dibenzofuranyl group, or a substituted or unsubstituted dibenzothiophenyl group, and R₁₂, R₁₄, R₂₂, and R₂₄ are preferably not a substituted or unsubstituted phenyl group.

In the compound according to the third exemplary embodiment, R₁₁ to R₁₄, R₂₁ to R₂₄, R₃₁ to R₃₄, R₄₁ to R₄₄, R₅₁ to R₅₄, and R₆₁ to R₆₄ more preferably each independently represent a hydrogen atom, an unsubstituted alkyl group having 1 to 8 carbon atoms, an unsubstituted phenyl group, an unsubstituted biphenyl group, an unsubstituted naphthyl group, an unsubstituted phenanthryl group, a substituted or unsubstituted fluorenyl group, an unsubstituted 9,9′-spirobifluorenyl group, an unsubstituted 9,9-dimethylfluorenyl group, an unsubstituted 9,9-diphenylfluorenyl group, an unsubstituted dibenzofuranyl group, or an unsubstituted dibenzothiophenyl group, and R₁₂, R₁₄, R₂₂, and R₂₄ are more preferably not a substituted or unsubstituted phenyl group.

In the compound according to the third exemplary embodiment, at least one combination of a combination of R₁₁ and R₁₂, a combination of R₁₃ and R₁₄, a combination of R₂₁ and R₂₂, or a combination of R₂₃ and R₂₄ is preferably mutually bonded to form a substituted or unsubstituted monocyclic ring or a substituted or unsubstituted fused ring, and at least one combination of adjacent two or more of R₃₁ to R₃₃, R₄₁ to R₄₃, R₅₁ to R₅₄, and R₆₁ to R₆₄ are preferably mutually bonded to form a substituted or unsubstituted monocyclic ring or a substituted or unsubstituted fused ring.

In the compound according to the third exemplary embodiment, a combination of R₁₁ and R₁₂, a combination of R₁₃ and R₁₄, a combination of R₂₁ and R₂₂, and a combination of R₂₃ and R₂₄ are also preferably not bonded mutually.

More specifically, in the compound according to the third exemplary embodiment, also preferably, a combination of R₁₁ and R₁₂ are not mutually bonded, a combination of R₁₃ and R₁₄ are not mutually bonded, a combination of R₂₁ and R₂₂ are not mutually bonded, and a combination of R₂₃ and R₂₄ are not mutually bonded.

In the compound according to the third exemplary embodiment, a combination of adjacent two or more of R₃₁ to R₃₃, R₄₁ to R₄₃, R₅₁ to R₅₄, and R₆₁ to R₆₄ are also preferably not bonded to each other.

The compound according to the third exemplary embodiment is preferably represented by one of the following formulae (1-2), (2-2) to (2-3), and (3-2) to (3-4).

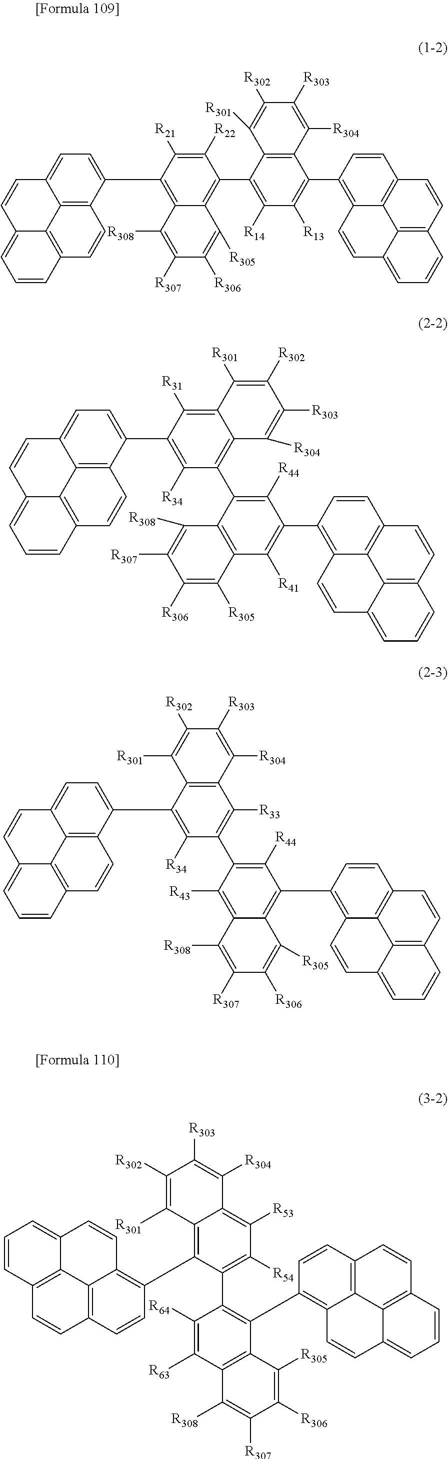

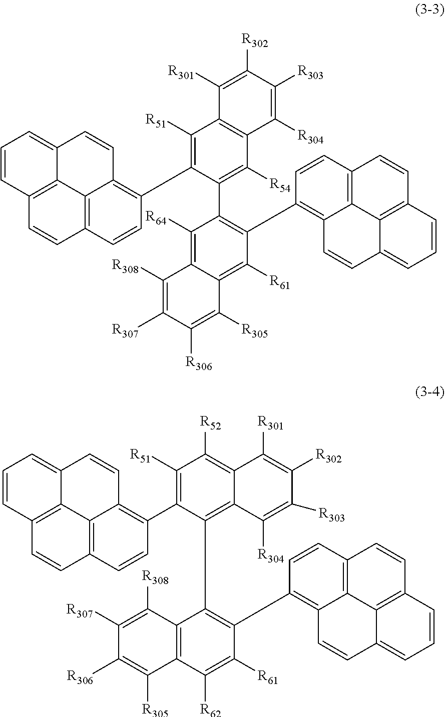

In the formulae (1-2), (2-2) to (2-3), and (3-2) to (3-4), R₁₃ to R₁₄, R₂₁ to R₂₂, R₃₁, R₃₃, R₃₄, R₄₁, R₄₃, R₄₄, R₅₁ to R₅₄, and R₆₁ to R₆₄ represent the same as R₁₃ to R₁₄, R₂₁ to R₂₂, R₃₁, R₃₃, R₃₄, R₄₁, R₄₃, R₄₄, R₅₁ to R₅₄, and R₆₁ to R₆₄, respectively, in the formulae (1) to (3), and R₃₀₁ to R₃₀₈ each independently represent the same as R₁₁ to R₁₄, R₂₁ to R₂₄, R₃₁ to R₃₄, R₄₁ to R₄₄, R₅₁ to R₅₄, and R₆₁ to R₆₄ in the formulae (1) to (3).

In the formulae (1-2), (2-2) to (2-3), and (3-2) to (3-4):
R₁₃ to R₁₄, R₂₁ to R₂₂, R₃₁, R₃₃, R₃₄, R₄₁, R₄₃, R₄₄, R₅₁ to R₅₄, R₆₁ to R₆₄, and R₃₀₁ to R₃₀₈ preferably each independently represent a hydrogen atom, a substituted or unsubstituted alkyl group having 1 to 8 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, a substituted or unsubstituted 9,9-dimethylfluorenyl group, a substituted or unsubstituted 9,9-diphenylfluorenyl group, a substituted or unsubstituted dibenzofuranyl group, or a substituted or unsubstituted dibenzothiophenyl group; and
R₁₄ and R₂₂ are preferably not a substituted or unsubstituted phenyl group.

In the formulae (1-2), (2-2) to (2-3), and (3-2) to (3-4):
R₁₃ to R₁₄, R₂₁ to R₂₂, R₃₁, R₃₃, R₃₄, R₄₁, R₄₃, R₄₄, R₅₁ to R₅₄, R₆₁ to R₆₄, and R₃₀₁ to R₃₀₈ preferably each independently represent a hydrogen atom, an unsubstituted alkyl group having 1 to 8 carbon atoms, an unsubstituted phenyl group, an unsubstituted biphenyl group, an unsubstituted naphthyl group, an unsubstituted phenanthryl group, a substituted or unsubstituted fluorenyl group, an unsubstituted 9,9′-spirobifluorenyl group, an unsubstituted 9,9-dimethylfluorenyl group, an unsubstituted 9,9-diphenylfluorenyl group, an unsubstituted dibenzofuranyl group, or an unsubstituted dibenzothiophenyl group; and
R₁₄ and R₂₂ are preferably not a substituted or unsubstituted phenyl group.

In the compound according to the third exemplary embodiment, a substituent for “substituted or unsubstituted” in R₁₁ to R₁₄, R₂₁ to R₂₄, R₃₁ to R₃₄, R₄₁ to R₄₄, R₅₁ to R₅₄, R₆₁ to R₆₄, R₁₁₁ to R₁₁₉, R₂₁₁ to R₂₁₉, and R₃₀₁ to R₃₀₈ is preferably each independently a substituted or unsubstituted alkyl group having 1 to 8 carbon atoms, a substituted or unsubstituted phenyl group, a substituted or unsubstituted naphthyl group, a substituted or unsubstituted phenanthryl group, a substituted or unsubstituted fluorenyl group, a substituted or unsubstituted dibenzofuranyl group, a substituted or unsubstituted dibenzothiophenyl group, a substituted or unsubstituted 9,9-dimethylfluorenyl group, or a substituted or unsubstituted 9,9-diphenylfluorenyl group.

In the compound according to the third exemplary embodiment, a substituent for “substituted or unsubstituted” in R₁₁ to R₁₄, R₂₁ to R₂₄, R₃₁ to R₃₄, R₄₁ to R₄₄, R₅₁ to R₅₄, R₆₁ to R₆₄, R₁₁₁ to R₁₁₉, R₂₁₁ to R₂₁₉, and R₃₀₁ to R₃₀₈ is preferably each independently an unsubstituted alkyl group having 1 to 8 carbon atoms, an unsubstituted phenyl group, an unsubstituted naphthyl group, an unsubstituted phenanthryl group, a substituted or unsubstituted fluorenyl group, an unsubstituted dibenzofuranyl group, an unsubstituted dibenzothiophenyl group, an unsubstituted 9,9-dimethylfluorenyl group, or an unsubstituted 9,9-diphenylfluorenyl group.

The compound according to the third exemplary embodiment in an emitting layer can improve luminous efficiency.

### Method for Producing Compound According to Third Exemplary Embodiment

The compound according to the third exemplary embodiment (a compound represented by the formula (1), (2), or (3)) can be produced by a known method. Furthermore, the compound according to the third exemplary embodiment can also be produced in accordance with a known method by using a known alternative reaction and raw materials suitable for the target compound.

Specific examples of the compound according to the third exemplary embodiment (a compound represented by the formula (1), (2), or (3)) include the following compounds. However, the invention is not limited to these specific examples.

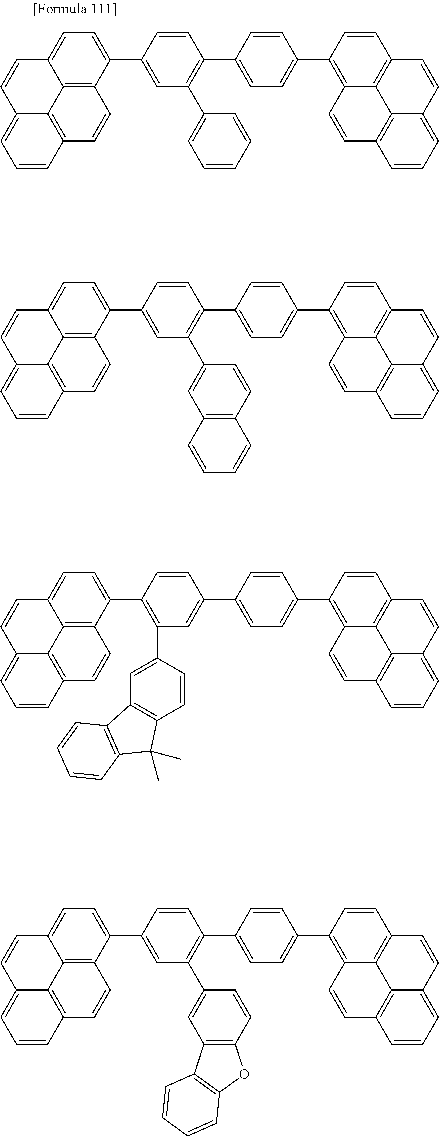

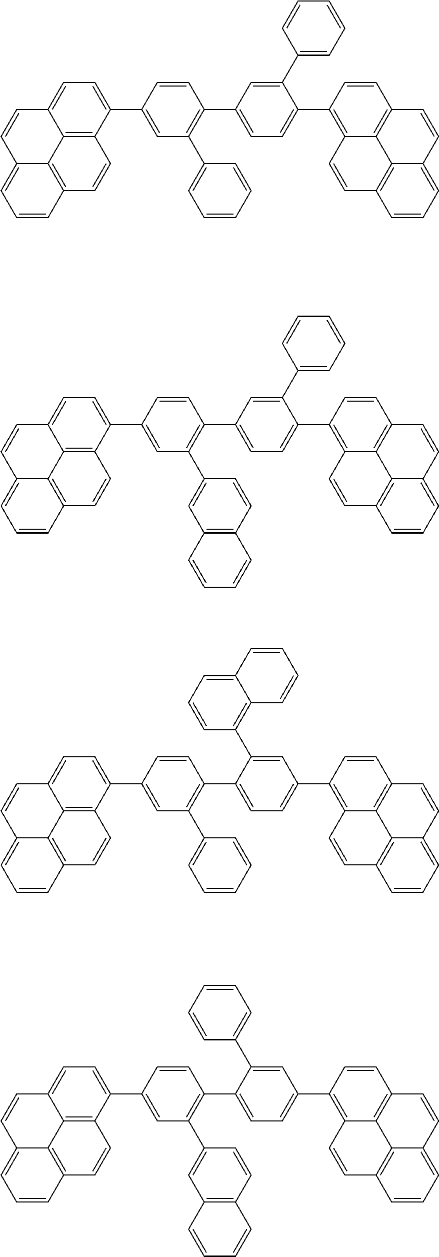

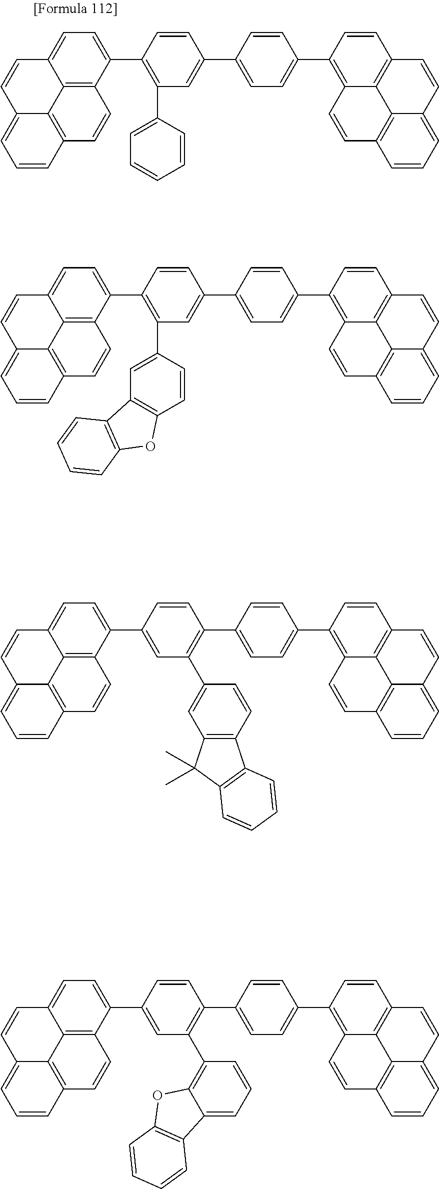

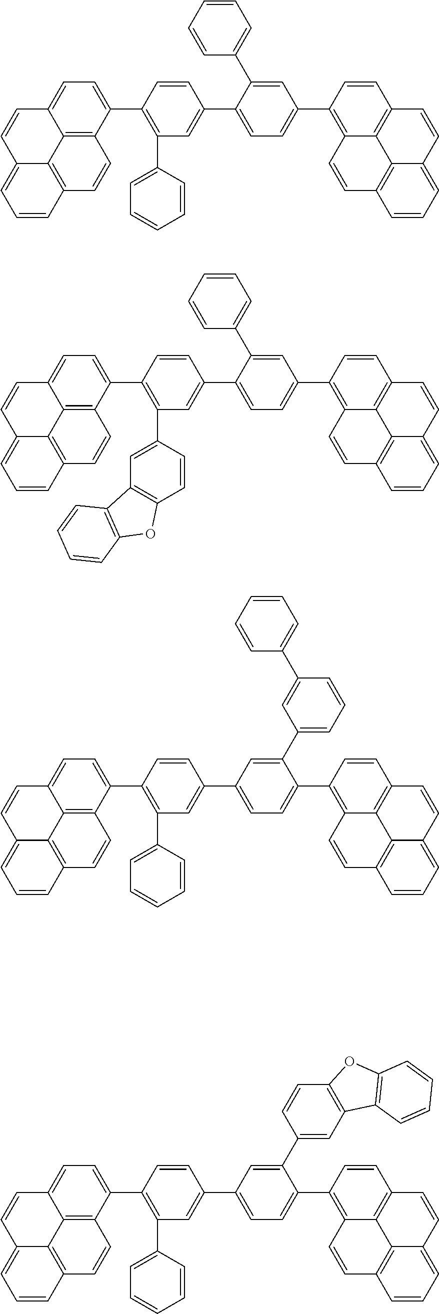

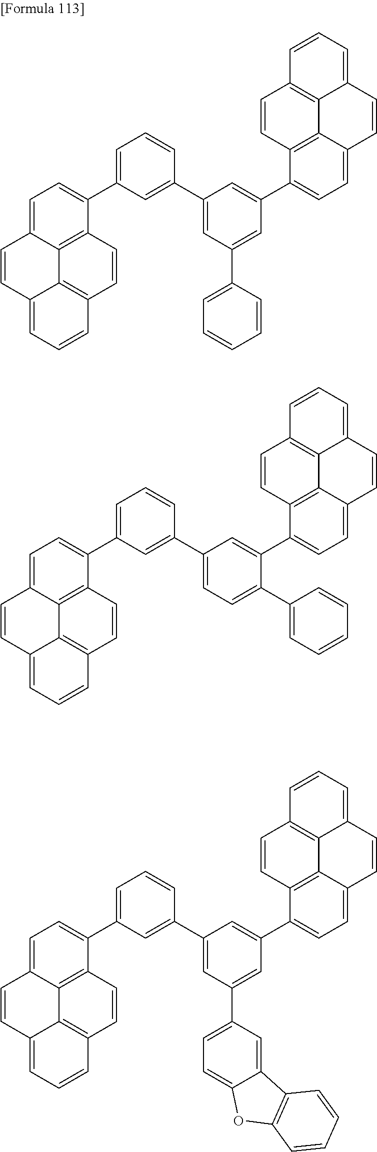

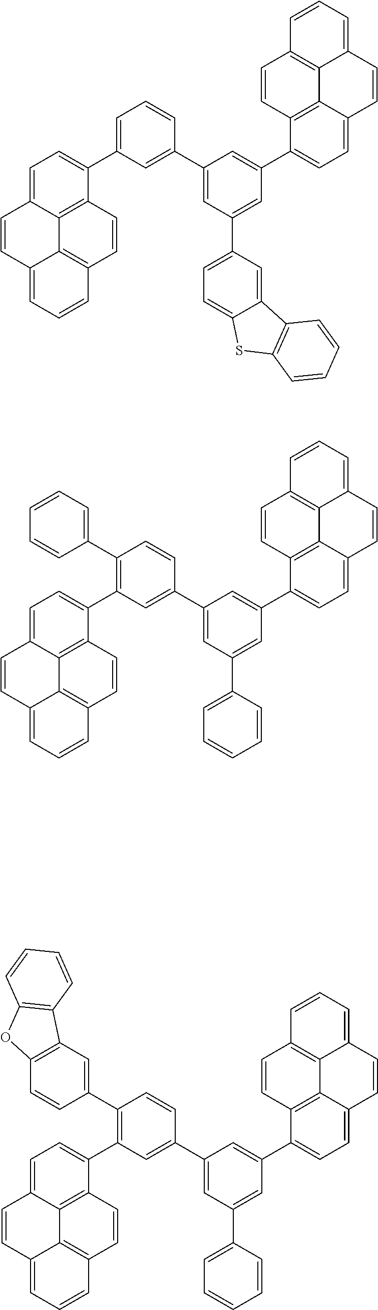

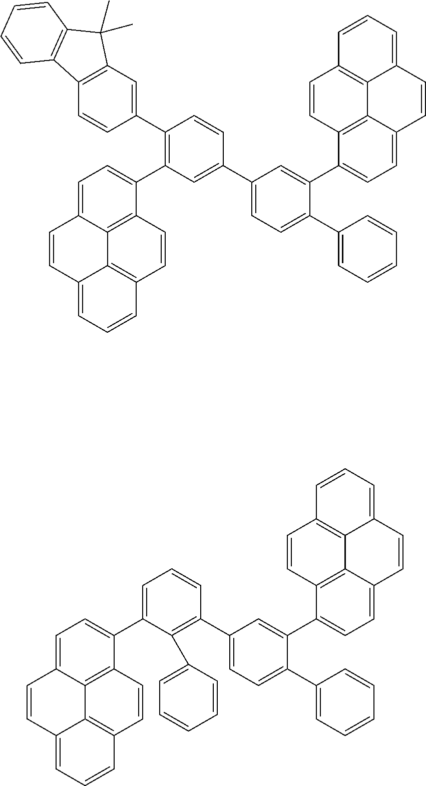

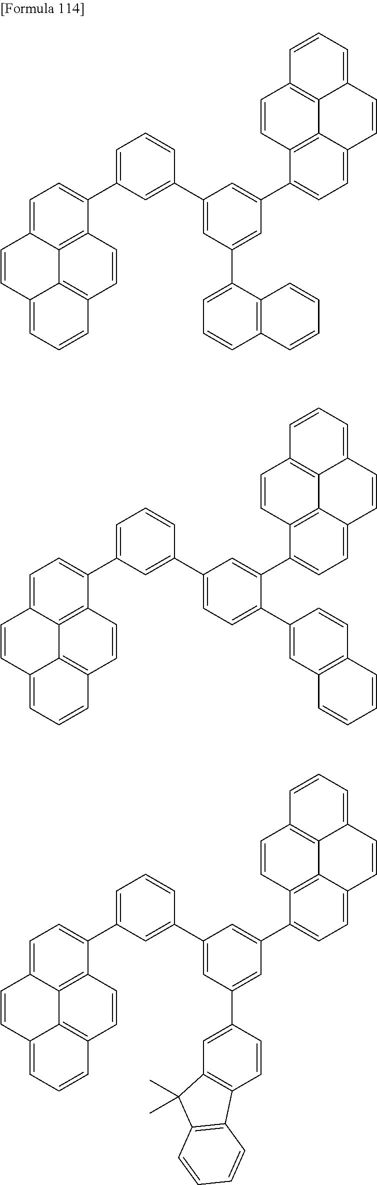

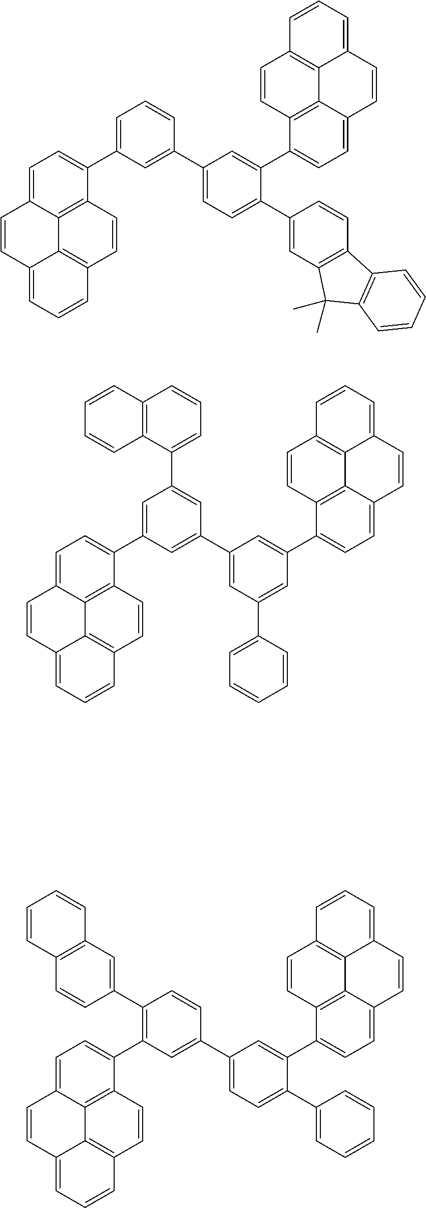

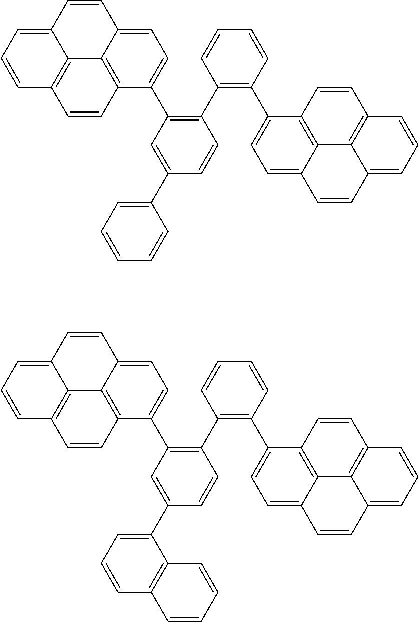

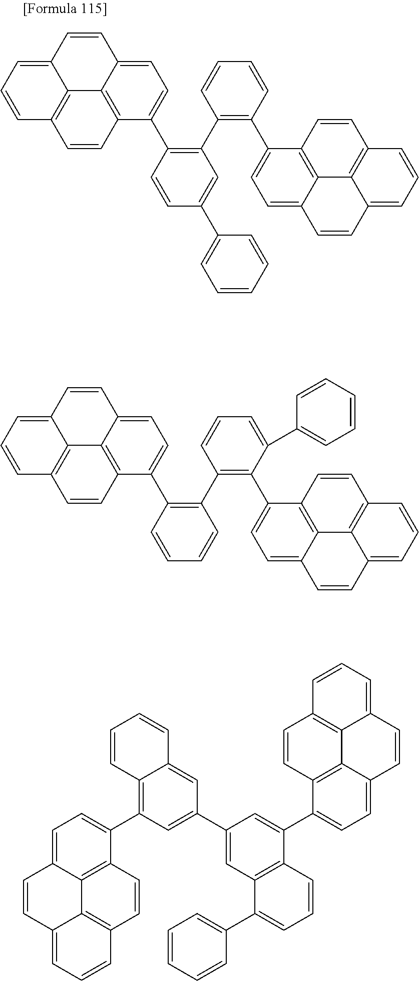

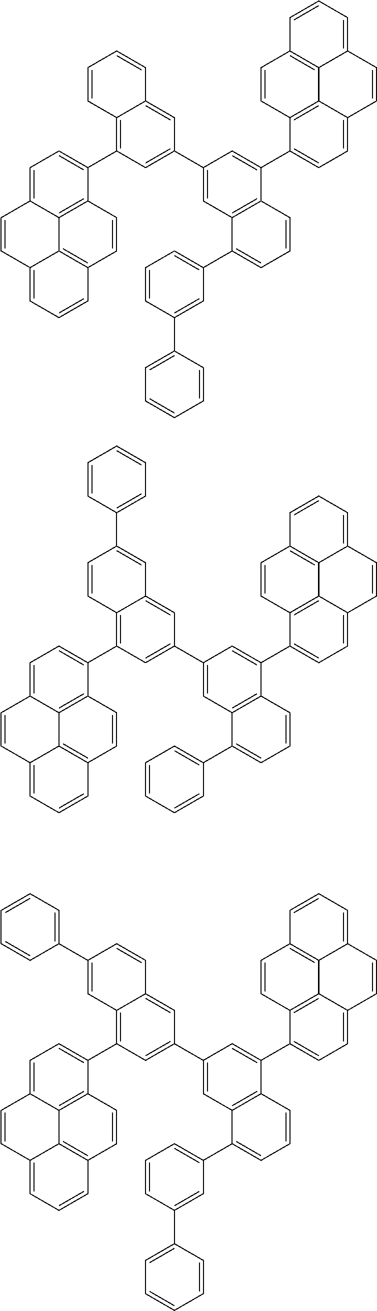

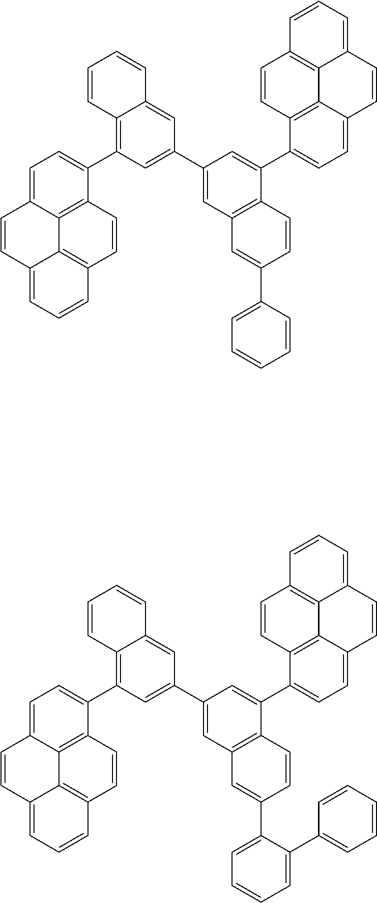

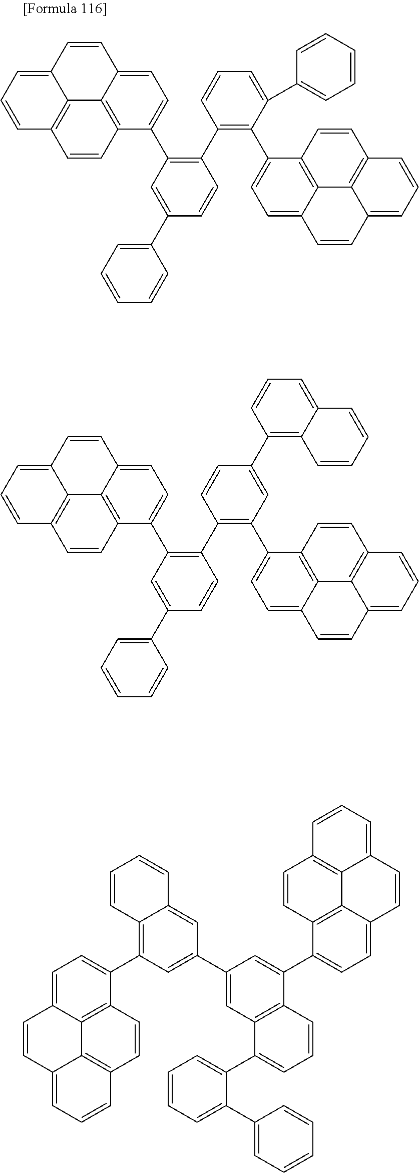

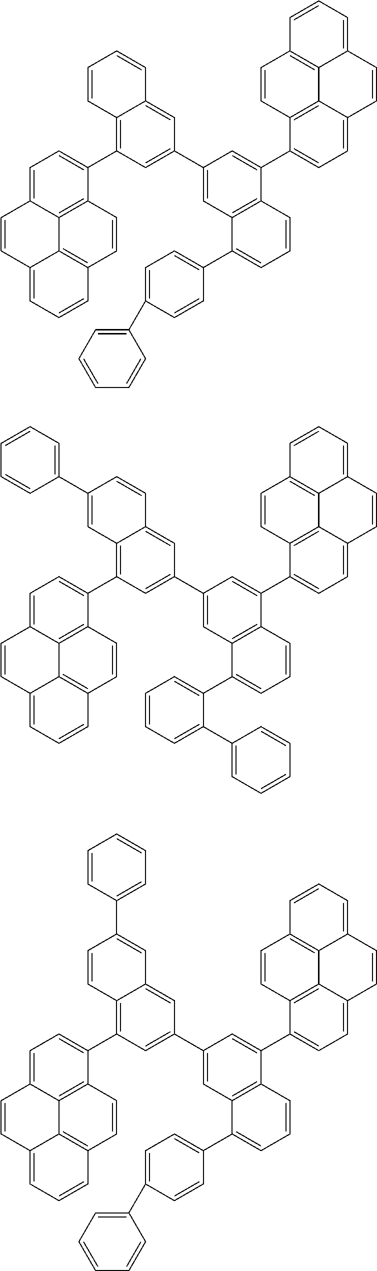

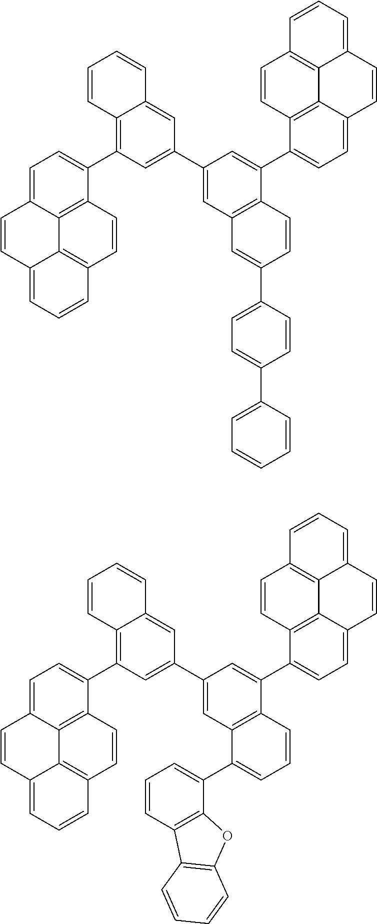

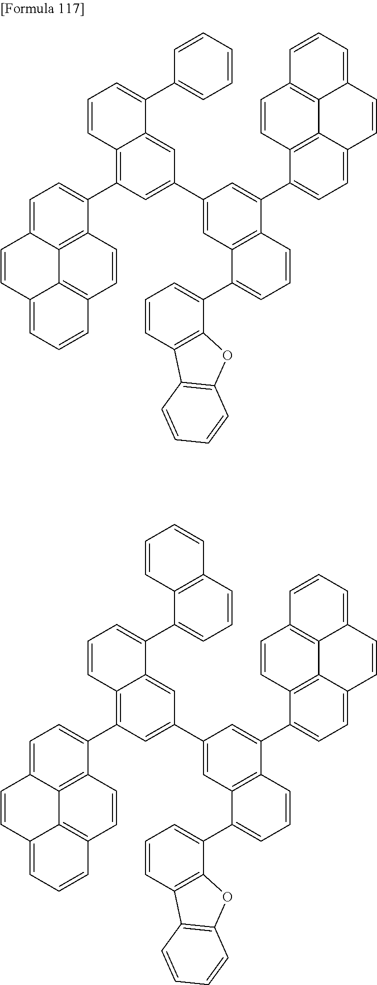 [SMILES: c1ccc(-c2cccc3c(-c4ccc5ccc6cccc7ccc4c5c67)cc(-c4cc(-c5ccc6ccc7cccc8ccc5c6c78)c5cccc(-c6cccc7c6oc6ccccc67)c5c4)cc23)cc1 ; c1ccc2c(-c3cccc4c(-c5ccc6ccc7cccc8ccc5c6c78)cc(-c5cc(-c6ccc7ccc8cccc9ccc6c7c89)c6cccc(-c7cccc8c7oc7ccccc78)c6c5)cc34)cccc2c1]

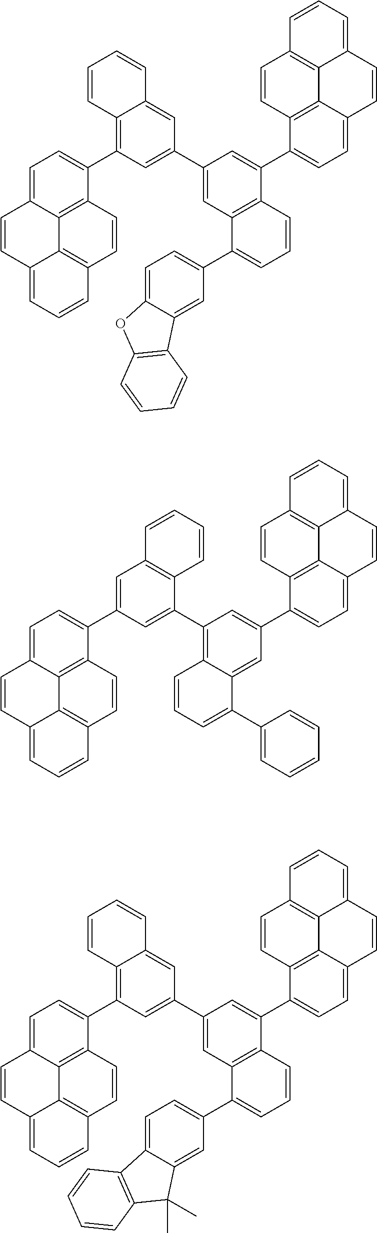

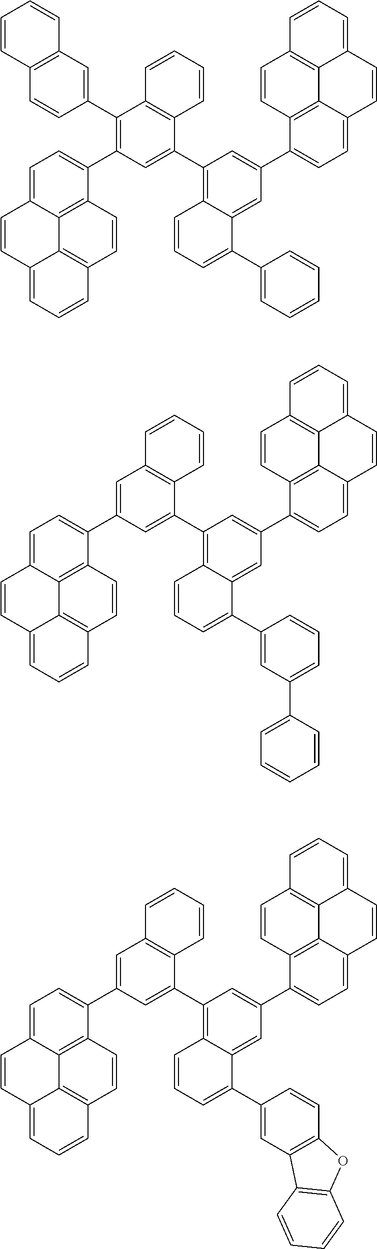

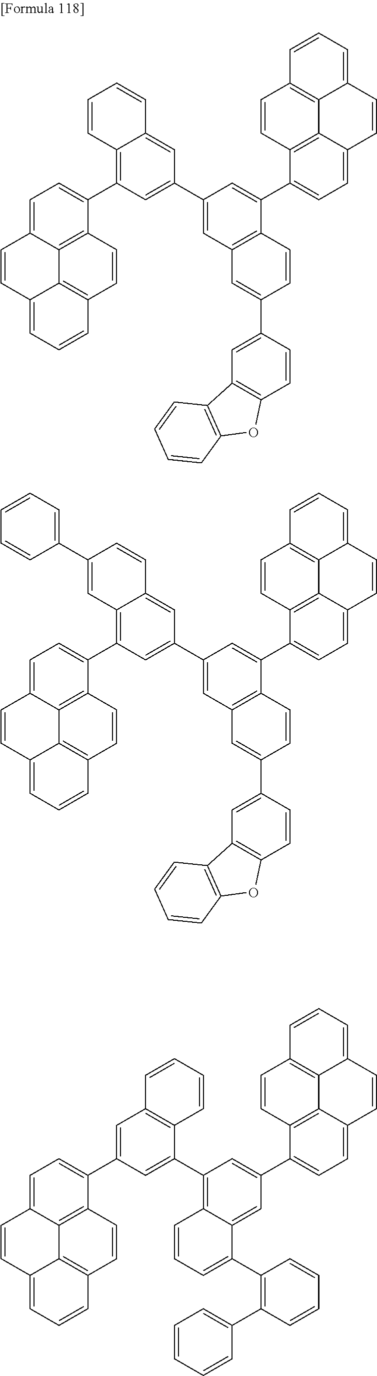

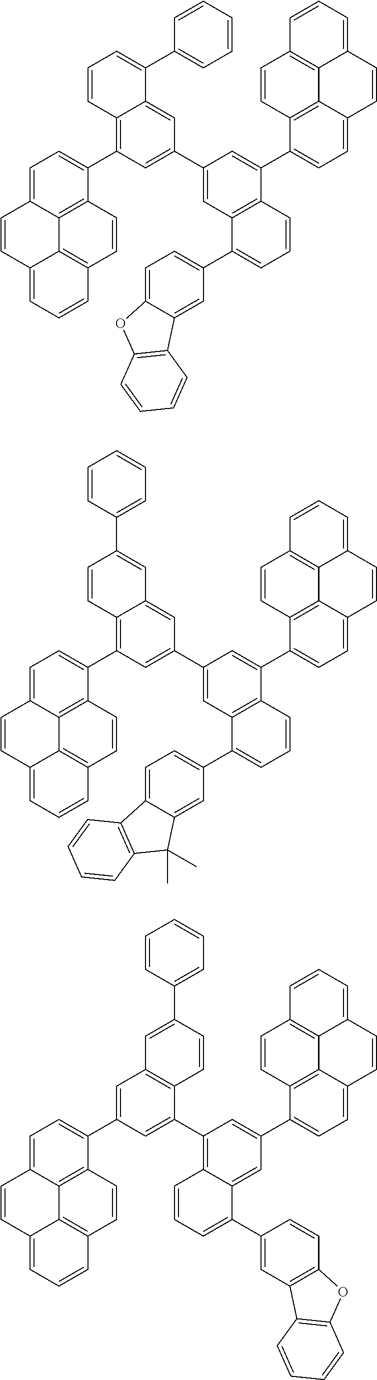

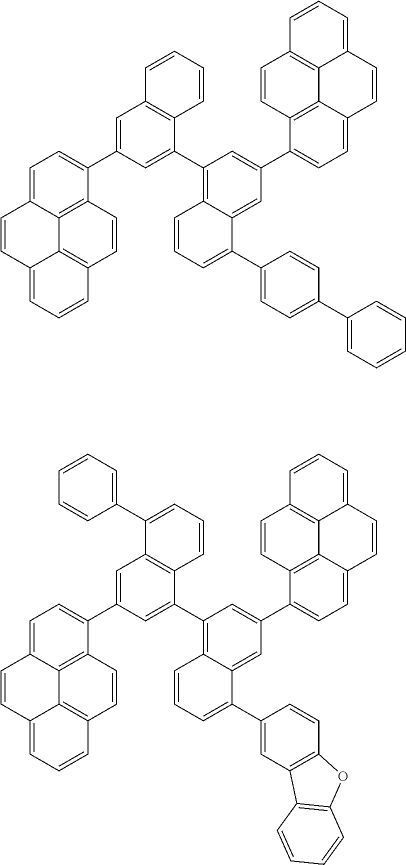

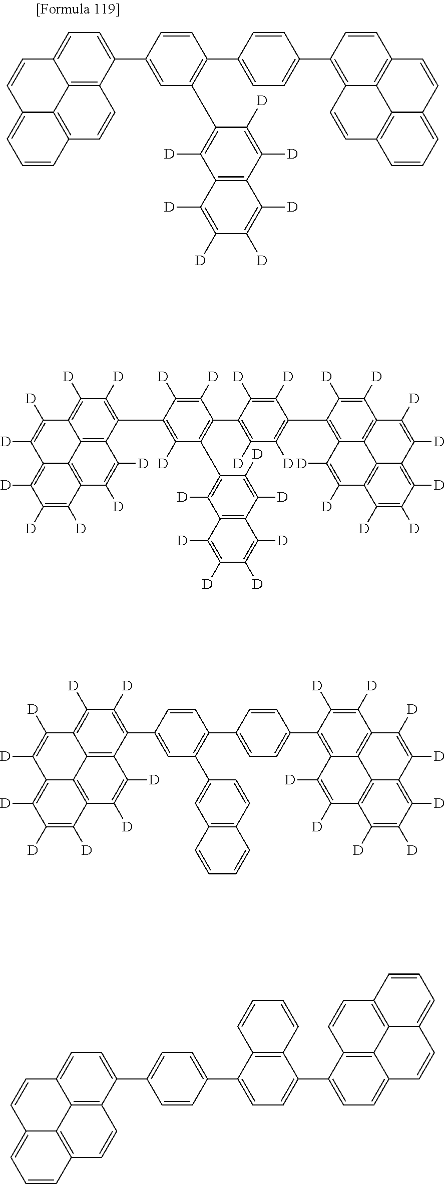

### Fourth Exemplary Embodiment

### Organic-EL-Device Material

An organic-EL-device material according to a fourth exemplary embodiment contains the compound according to the first exemplary embodiment (a compound represented by the formula (12X)), the compound according to the second exemplary embodiment (a compound represented by the formula (120)), or the compound according to the third exemplary embodiment (at least one of compounds represented by one of the formulae (1) to (3)).

The fourth exemplary embodiment provides an organic-EL-device material that can improve luminous efficiency.

The organic-EL-device material according to the fourth exemplary embodiment may further contain another compound. In the organic-EL-device material according to the fourth exemplary embodiment containing another compound, the other compound may be solid or liquid.

### Fifth Exemplary Embodiment

### Organic EL Device

The structure of an organic EL device according to a fifth exemplary embodiment is described below.

The organic EL device according to the fifth exemplary embodiment includes an anode, a cathode, and an emitting layer between the anode and the cathode.

The emitting layer contains the compound according to the first exemplary embodiment (a compound represented by the formula (12X)), the compound according to the second exemplary embodiment (a compound represented by the formula (120)), or the compound according to the third exemplary embodiment (at least one of compounds represented by one of the formulae (1) to (3)).

The emitting layer preferably contains the compound according to the first exemplary embodiment, the compound according to the second exemplary embodiment, or the compound according to the third exemplary embodiment as a host material.

Herein, the “host material” is a material constituting “50% or more by mass of the layer”, for example. Thus, for example, the emitting layer contains the compound according to the first exemplary embodiment, the compound according to the second exemplary embodiment, or the compound according to the third exemplary embodiment, which constitutes 50% or more by mass of the total mass of the emitting layer.

Emission Wavelength of Organic EL Device

The organic EL device according to the fifth exemplary embodiment preferably emits light with a main peak wavelength in the range of 430 to 480 nm when driven.

The main peak wavelength of light emitted from an organic EL device driven is measured as described below. While a voltage is applied to an organic EL device at an electric current density of 10 mA/cm², a spectral radiance spectrum is measured with a spectral radiance meter CS-2000 (manufactured by Konica Minolta, Inc. In a spectral radiance spectrum thus measured, the peak wavelength of an emission spectrum with a maximum luminous intensity is measured as a main peak wavelength (unit: nm).

The organic EL device according to the fifth exemplary embodiment may have at least one organic layer in addition to the emitting layer. The organic layer may be at least one layer selected from the group consisting of a hole injecting layer, a hole transporting layer, an electron injecting layer, an electron transporting layer, a hole blocking layer, and an electron blocking layer. The emitting layer may be composed of two or more layers.

In the organic EL device according to the fifth exemplary embodiment, the organic layer may consist of the emitting layer or may further have at least one layer selected from the group consisting of a hole injecting layer, a hole transporting layer, an electron injecting layer, an electron transporting layer, a hole blocking layer, an electron blocking layer, and the like.

The organic EL device according to the fifth exemplary embodiment preferably has a hole transporting layer between the anode and the emitting layer.

The organic EL device according to the fifth exemplary embodiment preferably has an electron transporting layer between the anode and the emitting layer.

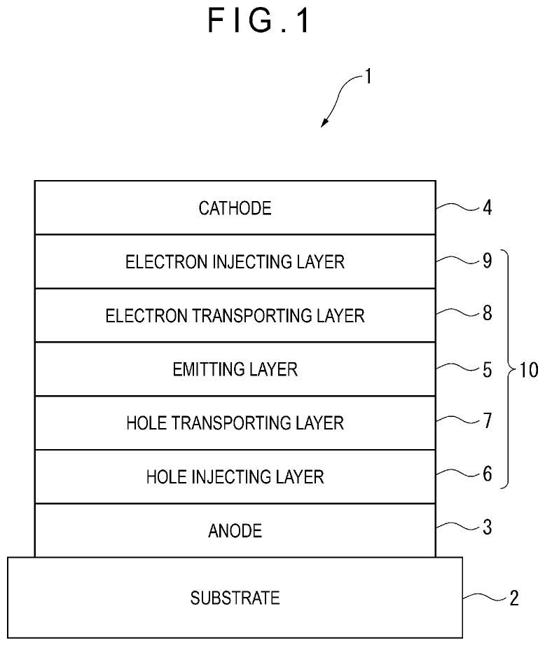

FIG. 1 is a schematic view of an example of the organic EL device according to the fifth exemplary embodiment.

An organic EL device 1 includes a light-transmitting substrate 2, an anode 3, a cathode 4, and an organic layer 10 between the anode 3 and the cathode 4. The organic layer 10 is composed of a hole injecting layer 6, a hole transporting layer 7, an emitting layer 5, an electron transporting layer 8, and an electron injecting layer 9 layered in this order on the side of the anode 3.

Fluorescent Compound

In the organic EL device 1 according to the fifth exemplary embodiment, the emitting layer 5 preferably further contains a fluorescent compound (hereinafter also referred to as a “compound M1”).

In the organic EL device 1 according to the fifth exemplary embodiment, the fluorescent compound (the compound M1) is preferably at least one compound selected from the group consisting of the compounds represented by the following formula (100), the compounds represented by the following formula (3), the compounds represented by the following formula (4), the compounds represented by the following formula (5), the compounds represented by the following formula (6), the compounds represented by the following formula (7), the compounds represented by the following formula (8), the compounds represented by the following formula (9), and the compounds represented by the following formula (10).

Compound Represented by Formula (100)

A compound represented by the formula (100) is described below.

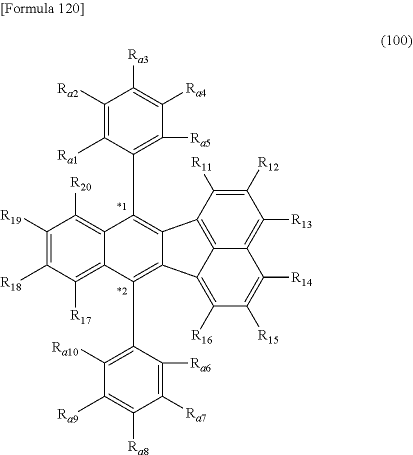

In the formula (100):
at least one combination of a combination of adjacent two or more of R₁₁ to R₁₆, a combination of adjacent two or more of R₁₇ to R₂₀, a combination of adjacent two or more of Rₐ₁ to Rₐ₅, and a combination of adjacent two or more of Rₐ₆ to Rₐ₁₀ are mutually bonded to form a substituted or unsubstituted monocyclic ring having 3 to 30 ring atoms, or form a substituted or unsubstituted fused ring having 3 to 30 ring atoms, or are not mutually bonded,
R₁₁ to R₂₀, Rₐ₁ to Rₐ₅, and Rₐ₆ to Rₐ₁₀ that do not form the substituted or unsubstituted monocyclic ring and do not form the substituted or unsubstituted fused ring each independently represent a hydrogen atom, a substituted or unsubstituted alkyl group having 1 to 30 carbon atoms, a substituted or unsubstituted cycloalkyl group having 3 to 30 ring carbon atoms, a substituted or unsubstituted alkoxy group having 1 to 30 carbon atoms, a substituted or unsubstituted alkylthio group having 1 to 30 carbon atoms, a substituted or unsubstituted amino group, a substituted or unsubstituted aryl group having 6 to 30 ring carbon atoms, a substituted or unsubstituted heterocyclic group having 5 to 30 ring atoms, a substituted or unsubstituted alkenyl group having 2 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted phosphanyl group, a substituted or unsubstituted phosphoryl group, a substituted or unsubstituted silyl group, a substituted or unsubstituted arylcarbonyl group having 6 to 30 ring carbon atoms, a cyano group, nitro group, carboxy group, or a halogen atom.

A specific example in which “at least one combination of a combination of adjacent two or more of R₁₁ to R₁₆, a combination of adjacent two or more of R₁₇ to R₂₀, a combination of adjacent two or more of Rₐ₁ to Rₐ₅, and a combination of adjacent two or more of Rₐ₆ to Rₐ₁₀” are mutually bonded to form a substituted or unsubstituted monocyclic ring or fused ring having 3 to 30 ring atoms is described below.

In a specific example in which a combination of adjacent two or more are mutually bonded to form a fused ring, in the case of R₁₇ to R₂₀ in the formula (100), a compound represented by the following formula (10A) is one example. In the compound represented by the following formula (10A), three adjacent R₁₁, R₁₉, and R₂₀ are mutually bonded to form a fused ring.

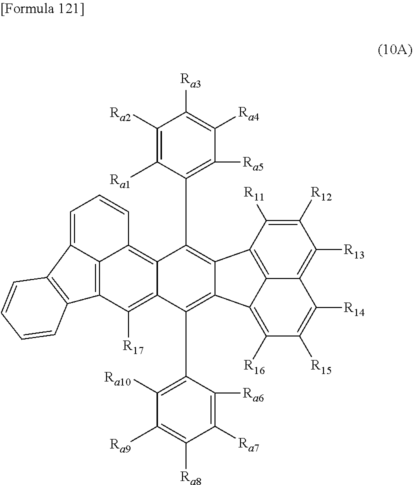

In the formula (10A), Rₐ₁ to Rₐ₁₀ and R₁₁ to R₁₇ represent the same as Rₐ₁ to Rₐ₁₀ and R₁₁ to R₁₇, respectively, in the formula (100).

In a specific example in which a combination of adjacent two or more are mutually bonded to form a monocyclic ring, for R₁₁ to R₁₆ in the formula (100), a compound represented by the following formula (10B) is one example. In the compound represented by the following formula (10B), a combination of R₁₂ and R₁₃ and a combination of R₁₄ and R₁₅ are independently bonded to each other to form two monocyclic rings.

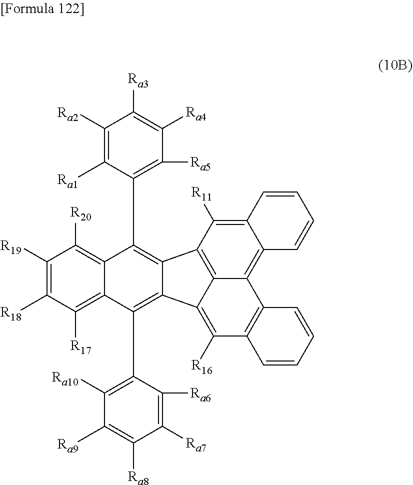

In the formula (10B), Rₐ₁ to Rₐ₁₀, R₁₁, and R₁₆ to R₂₀ represent the same as Rₐ₁ to Rₐ₁₀, R₁₁, and R₁₆ to R₂₀, respectively, in the formula (100).

In an exemplary embodiment, a compound represented by the formula (100) is a compound represented by the following formula (10-1).

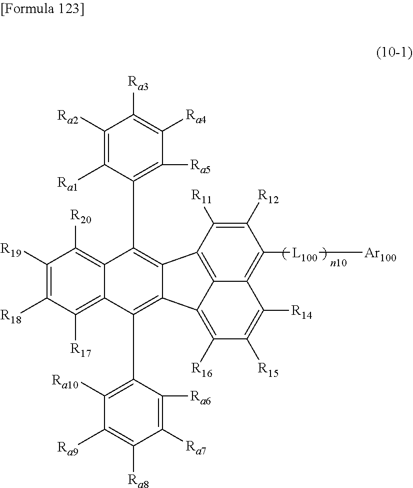

In the formula (10-1):
R₁₁ to R₁₂, R₁₄ to R₂₀, and Rₐ₁ to Rₐ₁₀ represent the same as R₁₁ to R₁₂, R₁₄ to R₂₀, and Rₐ₁ to Rₐ₁₀, respectively, in the formula (100),
n₁₀ represents 0, 1, 2, or 3,
L₁₀₀ represents a substituted or unsubstituted arylene group having 6 to 50 ring carbon atoms or a substituted or unsubstituted divalent heterocyclic group having 5 to 50 ring atoms,
when n₁₀ is 2, a plurality of L₁₀₀ are mutually the same or different,
when n₁₀ is 3, a plurality of L₁₀₀ are mutually the same or different, and
Ar₁₀₀ represents a substituted or unsubstituted aryl group having 6 to 50 ring carbon atoms, a substituted or unsubstituted heterocyclic group having 5 to 50 ring atoms, a cyano group, or a substituted silyl group.

In the formula (10-1), n₁₀ preferably represents 0, 1, or 2.

In the formula (10-1), L₁₀₀ preferably represents a divalent group derived by removing one hydrogen atom from a group selected from the group consisting of a single bond, a substituted or unsubstituted phenyl group, substituted or unsubstituted biphenyl group, substituted or unsubstituted terphenyl group, substituted or unsubstituted naphthyl group, substituted or unsubstituted anthryl group, substituted or unsubstituted phenanthryl group, substituted or unsubstituted fluorenyl group, substituted or unsubstituted 9,9′-spirobifluorenyl group, substituted or unsubstituted 9,9-dimethylfluorenyl group, substituted or unsubstituted 9,9-diphenylfluorenyl group, substituted or unsubstituted dibenzofuranyl group, substituted or unsubstituted naphthobenzofuranyl group, substituted or unsubstituted dibenzothiophenyl group, and substituted or unsubstituted naphthobenzothiophenyl group.

Ar₁₀₀ in the formula (10-1) preferably represents a cyano group, a substituted silyl group, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted fluorenyl group, a substituted or unsubstituted 9,9′-spirobifluorenyl group, a substituted or unsubstituted 9,9-dimethylfluorenyl group, a substituted or unsubstituted 9,9-diphenylfluorenyl group, a substituted or unsubstituted dibenzofuranyl group, a substituted or unsubstituted naphthobenzofuranyl group, a substituted or unsubstituted dibenzothiophenyl group, or a substituted or unsubstituted naphthobenzothiophenyl group.

In the formula (10-1), at least one combination of a combination of adjacent two or more of R₁₁ to R₁₂, R₁₄ to R₁₆, and R₁₇ to R₂₀, a combination of adjacent two or more of Rₐ₁ to Rₐ₅, and a combination of adjacent two or more of Rₐ₆ to Rₐ₁₀ are mutually bonded to form a substituted or unsubstituted monocyclic ring having 3 to 30 ring atoms or form a substituted or unsubstituted fused ring having 3 to 30 ring atoms, or are not mutually bonded.

In an exemplary embodiment, R₁₂ and R₁₃ in the formula (100) are mutually bonded to form a substituted or unsubstituted monocyclic ring having 3 to 30 ring atoms or form a substituted or unsubstituted fused ring having 3 to 30 ring atoms.

In an exemplary embodiment, a compound represented by the formula (100) is a compound represented by the following formula (10-2).

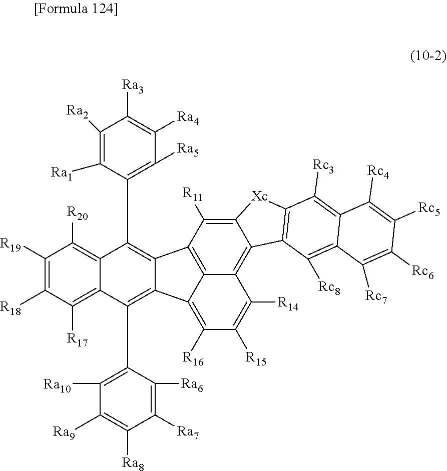

In the formula (10-2),
Xc represents an oxygen atom, a sulfur atom, or CR_{c1}R_{c2},
R₁₁, R₁₄ to R₂₀, Rₐ₁ to Rₐ₁₀, and R_{c3} to Rea each independently represent the same as R₁₁ to R₂₀ in the formula (100),
R_{c1} and R_{c2} each independently represent a hydrogen atom, an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, or an unsubstituted having 5 to 50 ring atoms monovalent heterocyclic group.
R₉₀₁ to R₉₀₇ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms.

When a plurality of R₉₀₁ are present, the plurality of R₉₀₁ are mutually the same or different. When a plurality of R₉₀₂ are present, the plurality of R₉₀₂ are mutually the same or different. When a plurality of R₉₀₃ are present, the plurality of R₉₀₃ are mutually the same or different. When a plurality of R₉₀₄ are present, the plurality of R₉₀₄ are mutually the same or different. When a plurality of R₉₀₅ are present, the plurality of R₉₀₅ are mutually the same or different. When a plurality of R₉₀₆ are present, the plurality of R₉₀₆ are mutually the same or different. When a plurality of R₉₀₇ are present, the plurality of R₉₀₇ are mutually the same or different.

In the formula (10-2), at least one combination of a combination of adjacent two or more of R₁₄ to R₁₆ and R₁₇ to R₂₀, a combination of adjacent two or more of Rₐ₁ to Rₐ₅, a combination of adjacent two or more of Rₐ₆ to Rₐ₁₀, a combination of adjacent two or more of R_{c3} to R_{c8}, and a combination of R_{c1} and R_{c2} are mutually bonded to form a substituted or unsubstituted monocyclic ring having 3 to 30 ring atoms or form a substituted or unsubstituted fused ring having 3 to 30 ring atoms, or are not mutually bonded.

In an exemplary embodiment, two or more of R₁, to R₂₀ in the formula (100) are mutually bonded to form a substituted or unsubstituted monocyclic ring having 3 to 30 ring atoms or form a substituted or unsubstituted fused ring having 3 to 30 ring atoms.

In an exemplary embodiment, a compound represented by the formula (100) is a compound represented by the following formula (10-3).

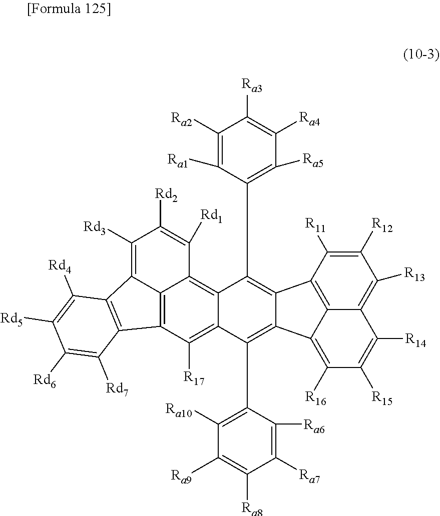

In the formula (10-3), R₁₁ to R₁₇, Rₐ₁ to Rₐ₁₀, and R_{d1} to R_{d7} each independently represent the same as R₁₁ to R₂₀ in the formula (100).

In the formula (10-3), at least one combination of a combination of adjacent two or more of R₁₁ to R₁₆, a combination of adjacent two or more of R₁₇ and R_{d1} to R_{d7}, a combination of adjacent two or more of Rₐ₁ to Rₐ₅, and a combination of adjacent two or more of Rₐ₆ to Rₐ₁₀ are mutually bonded to form a substituted or unsubstituted monocyclic ring having 3 to 30 ring atoms or form a substituted or unsubstituted fused ring having 3 to 30 ring atoms, or are not mutually bonded.

In an exemplary embodiment, R₁₁ to R₂₀, Rₐ₁ to Rₐ₅, Rₐ₆ to Rₐ₁₀, R_{c1} to R_{c8}, and R_{d1} to R_{d7} that are not involved in ring formation in the formulae (100) and (10-1) to (10-3) each independently represent a hydrogen atom, an unsubstituted aryl group having 6 to 50 ring carbon atoms, or an unsubstituted monovalent heterocyclic group having 5 to 50 ring atoms.

Specific examples of a compound represented by the formula (100) include the following compounds.

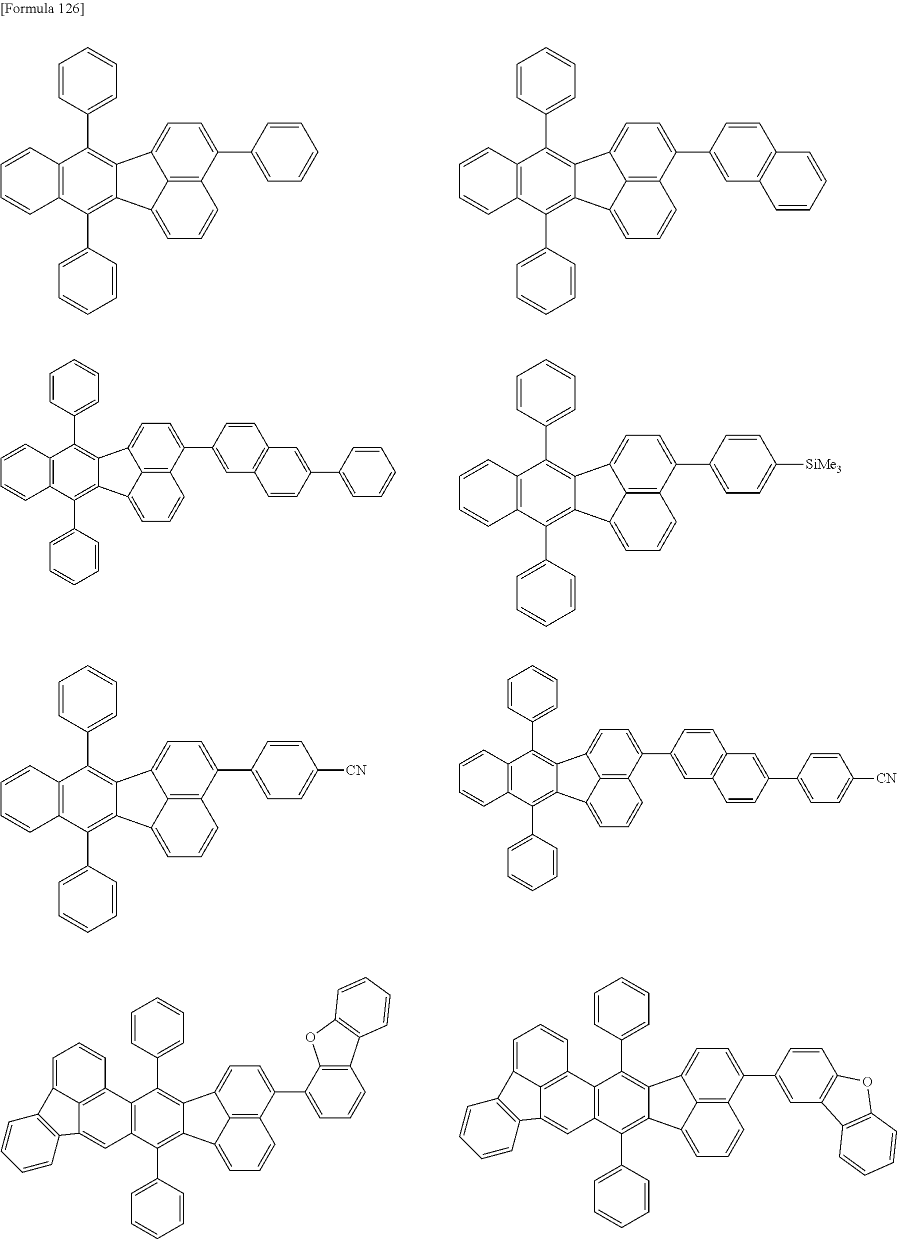

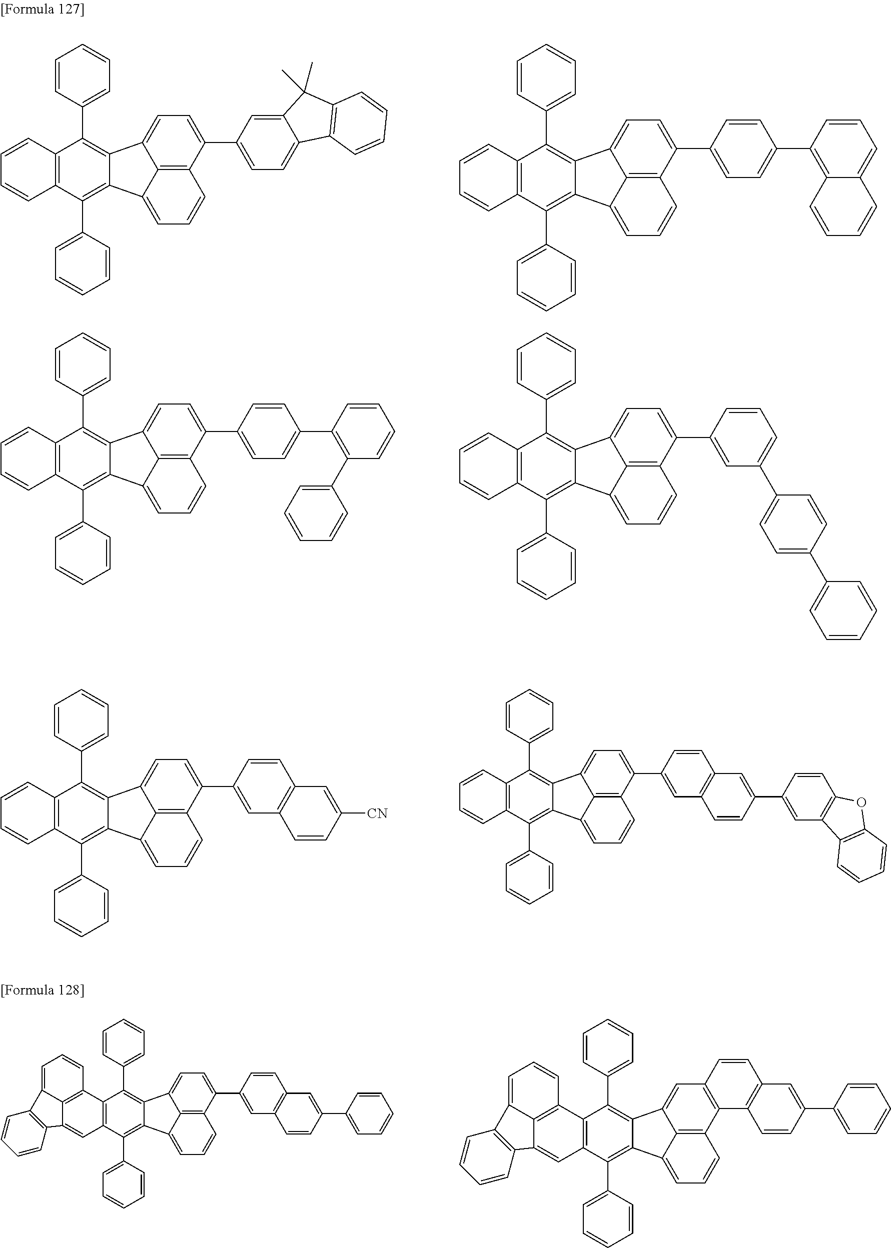

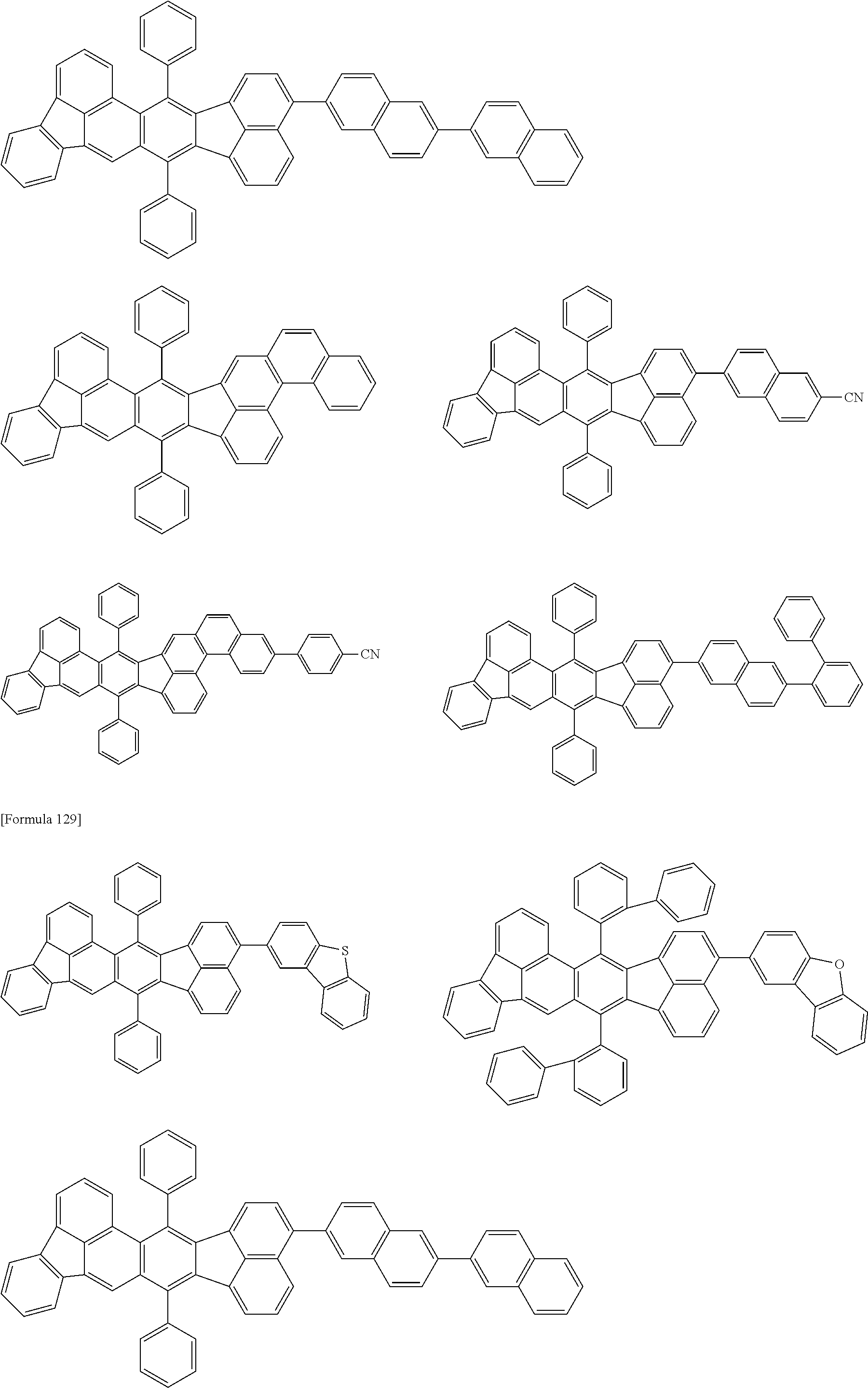

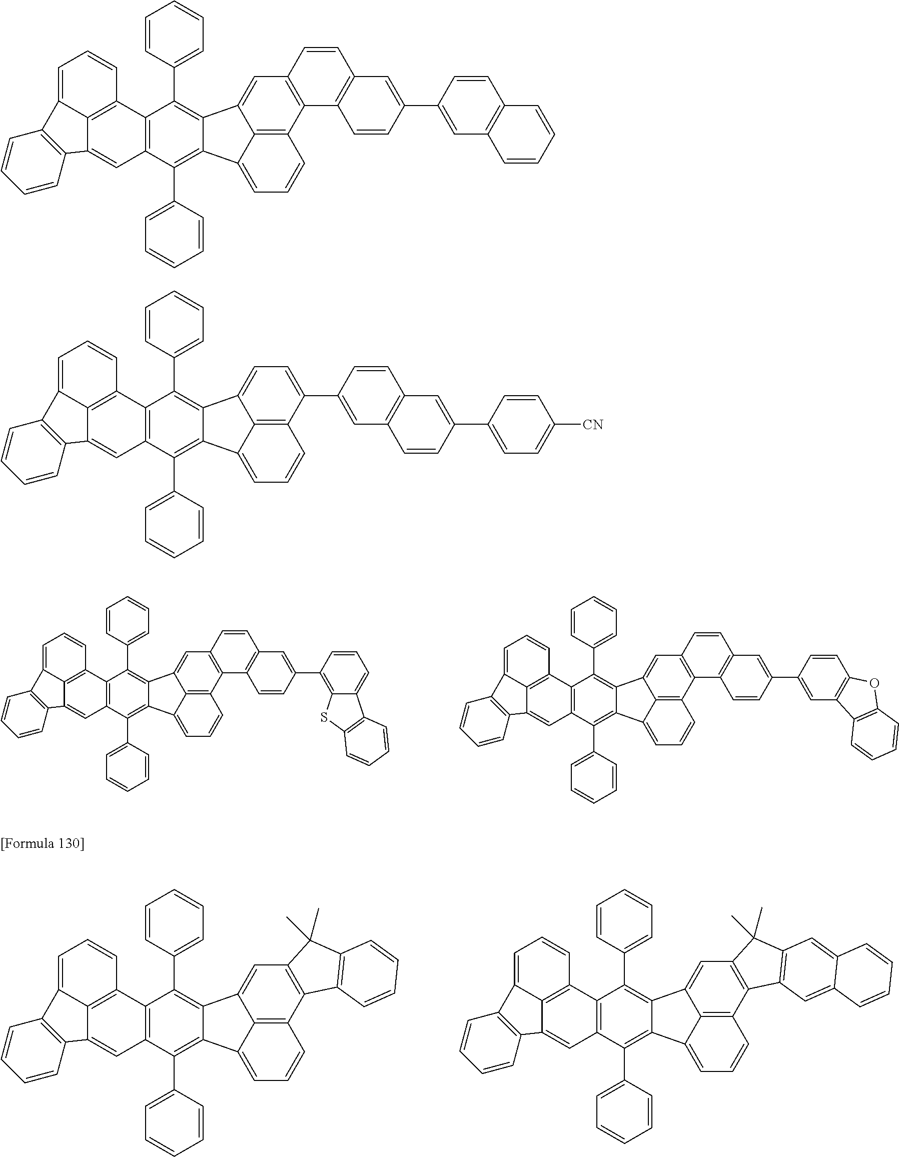

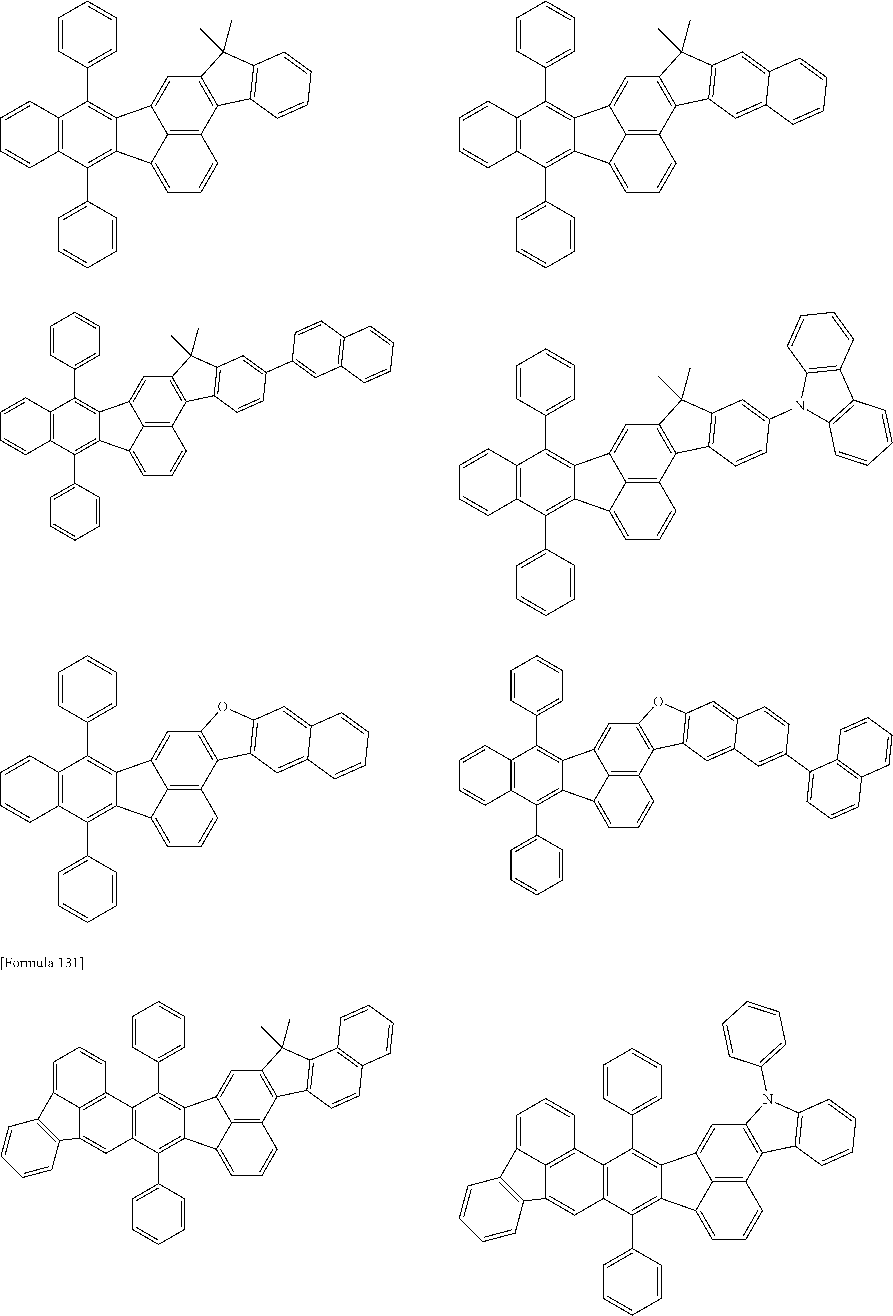

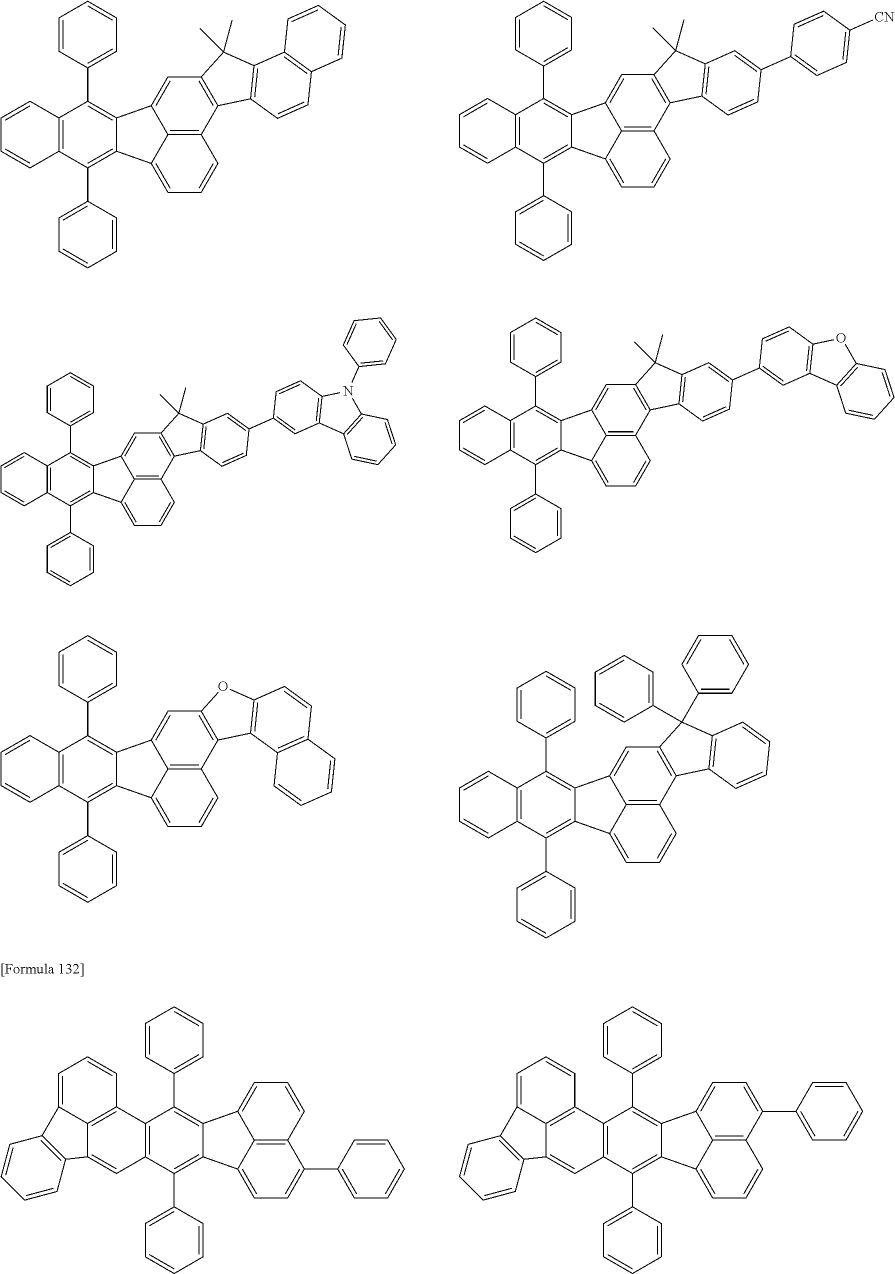

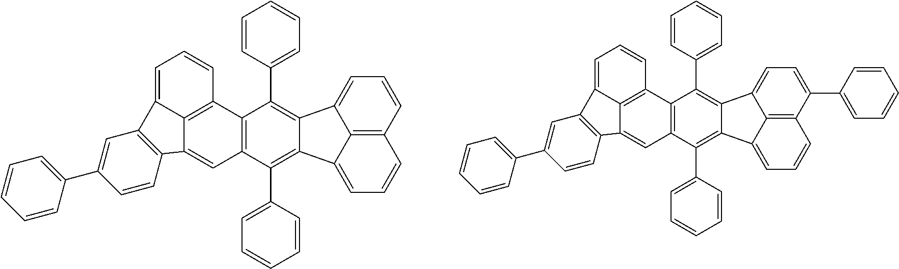

(Compound Represented by Formula (3))

A compound represented by the formula (3) is described below.

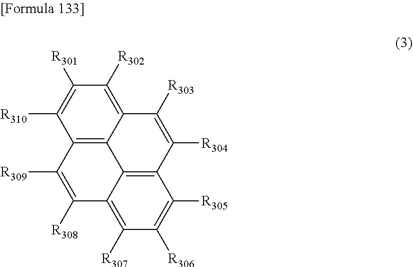

In the formula (3),
at least one combination of adjacent two or more of R₃₀₁ to R₃₁₀ are mutually bonded to form a substituted or unsubstituted monocyclic ring, are mutually bonded to form a substituted or unsubstituted fused ring, or are not mutually bonded,
at least one of R₃₀₁ to R₃₁₀ represents a monovalent group represented by the following formula (31), and
R₃₀₁ to R₃₁₀ that do not form the monocyclic ring, do not form the fused ring, and are not monovalent group represented by the following formula (31) each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

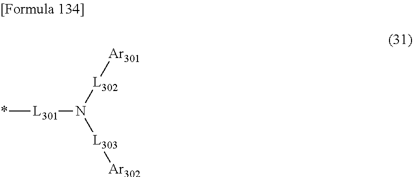

In the formula (31),
Ar₃₀₁ and Ar₃₀₂ each independently represent a substituted or unsubstituted aryl group having 6 to 50 ring carbon atoms, or a substituted or unsubstituted heterocyclic group having 5 to 50 ring atoms,
L₃₀₁ to L₃₀₃ each independently represent a single bond, a substituted or unsubstituted arylene group having 6 to 30 ring carbon atoms, or a substituted or unsubstituted divalent heterocyclic group having 5 to 30 ring atoms, and
* represents the bonding position of the pyrene ring in the formula (3).

In the fluorescent compound (the compound M1), R₉₀₁, R₉₀₂, R₉₀₃, R₉₀₄, R₉₀₅, R₉₀₆, and R₉₀₇ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted aryl group having 6 to 50 ring carbon atoms,
when a plurality of R₉₀₁ are present, the plurality of R₉₀₁ are mutually the same or different,
when a plurality of R₉₀₂ are present, the plurality of R₉₀₂ are mutually the same or different,
when a plurality of R₉₀₃ are present, the plurality of R₉₀₃ are mutually the same or different,
when a plurality of R₉₀₄ are present, the plurality of R₉₀₄ are mutually the same or different,
when a plurality of R₉₀₅ are present, the plurality of R₉₀₅ are mutually the same or different,
when a plurality of R₉₀₆ are present, the plurality of R₉₀₆ are mutually the same or different, and
when a plurality of R₉₀₇ are present, the plurality of R₉₀₇ are mutually the same or different.

In the formula (3), two of R₃₀₁ to R₃₁₀ preferably represent a group represented by the formula (31).

In an exemplary embodiment, a compound represented by the formula (3) is a compound represented by the following formula (33).

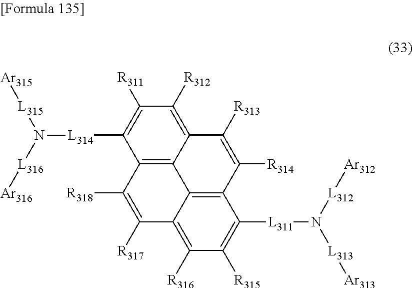

In the formula (33),
R₃₁₁ to R₃₁₈ each independently represent the same as R₃₀₁ to R₃₁₀ in the formula (3) that are not monovalent groups represented by the formula (31),
L₃₁₁ to L₃₁₆ each independently represent a single bond, a substituted or unsubstituted arylene group having 6 to 30 ring carbon atoms, or a substituted or unsubstituted divalent heterocyclic group having 5 to 30 ring atoms, and
Ar₃₁₂, Ar₃₁₃, Ar₃₁₅, and Ar₃₁₆ each independently represent a substituted or unsubstituted aryl group having 6 to 50 ring carbon atoms, or a substituted or unsubstituted heterocyclic group having 5 to 50 ring atoms.

In the formula (31), L₃₀₁ preferably represents a single bond, and L₃₀₂ and L₃₀₃ preferably represent a single bond.

In an exemplary embodiment, a compound represented by the formula (3) is represented by the following formula (34) or (35).

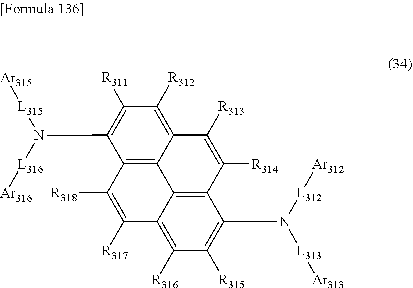

In the formula (34),
R₃₁₁ to R₃₁₈ each independently represent the same as R₃₀₁ to R₃₁₀ in the formula (3) that are not monovalent groups represented by the formula (31),
L₃₁₂, L₃₁₃, L₃₁₅, and L₃₁₆ represent the same as L₃₁₂, L₃₁₃, L₃₁₅, and L₃₁₆, respectively, in the formula (33), and
Ar₃₁₂, Ar₃₁₃, Ar₃₁₅, and Ar₃₁₆ represent the same as Ar₃₁₂, Ar₃₁₃, Ar₃₁₅, and Ar₃₁₆, respectively, in the formula (33).

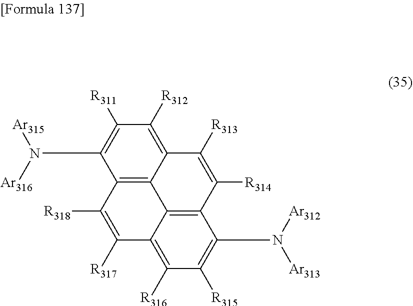

In the formula (35),
R₃₁₁ to R₃₁₈ each independently represent the same as R₃₀₁ to R₃₁₀ in the formula (3) that are not monovalent groups represented by the formula (31), and
Ar₃₁₂, Ar₃₁₃, Ar₃₁₅, and Ar₃₁₆ represent the same as Ar₃₁₂, Ar₃₁₃, Ar₃₁₅, and Ar₃₁₆, respectively, in the formula (33).

In the formula (31), preferably, at least one of Ar₃₀₁ or Ar₃₀₂ represents a group represented by the following formula (36).

In the formulae (33) to (35), preferably, at least one of Ar₃₁₂ or Ar₃₁₃ represents a group represented by the following formula (36).

In the formulae (33) to (35), preferably, at least one of Ar₃₁₅ or Ar₃₁₆ represents a group represented by the following formula (36).

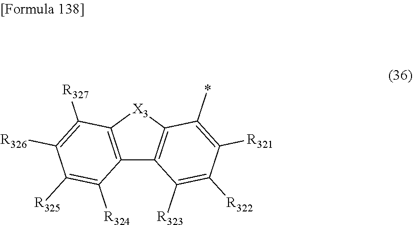

In the formula (36),
X₃ represents an oxygen atom or a sulfur atom,
at least one combination of adjacent two or more of R₃₂₁ to R₃₂₇ are mutually bonded to form a substituted or unsubstituted monocyclic ring, are mutually bonded to form a substituted or unsubstituted fused ring, or are not mutually bonded,
R₃₂₁ to R₃₂₇ that do not form the monocyclic ring and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms, and
* represents a position of bonding to L₃₀₂, L₃₀₃, L₃₁₂, L₃₁₃, L₃₁₅, or L₃₁₆.
X₃ preferably represents an oxygen atom.

At least one of R₃₂₁ to R₃₂₇ preferably represents a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31), Ar₃₀₁ preferably represents a group represented by the formula (36), and Ar₃₀₂ preferably represents a substituted or unsubstituted aryl group having 6 to 50 ring carbon atoms.

In the formulae (33) to (35), Ar₃₁₂ preferably represents a group represented by the formula (36), and Ar₃₁₃ preferably represents a substituted or unsubstituted aryl group having 6 to 50 ring carbon atoms.

In the formulae (33) to (35), Ar₃₁₅ preferably represents a group represented by the formula (36), Ar₃₁₆ preferably represents a substituted or unsubstituted aryl group having 6 to 50 ring carbon atoms.

In an exemplary embodiment, a compound represented by the formula (3) is represented by the following formula (37).

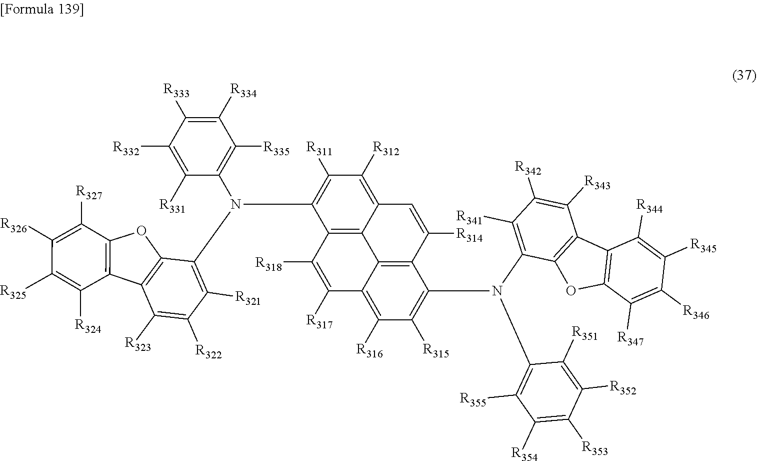

In the formula (37),
R₃₁₁ to R₃₁₈ each independently represent the same as R₃₀₁ to R₃₁₀ in the formula (3) that are not monovalent groups represented by the formula (31),
at least one combination of adjacent two or more of R₃₂₁ to R₃₂₇
are mutually bonded to form a substituted or unsubstituted monocyclic ring,
are mutually bonded to form a substituted or unsubstituted fused ring, or
are not mutually bonded,
at least one combination of adjacent two or more of R₃₄₁ to R₃₄₇ are mutually bonded to form a substituted or unsubstituted monocyclic ring, are mutually bonded to form a substituted or unsubstituted fused ring, or are not mutually bonded,
R₃₂₁ to R₃₂₇ and R₃₄₁ to R₃₄₇ that do not form the monocyclic ring and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms, and
R₃₃₁ to R₃₃₅ and R₃₅₁ to R₃₅₅ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of a compound represented by the formula (3) include the following compounds.

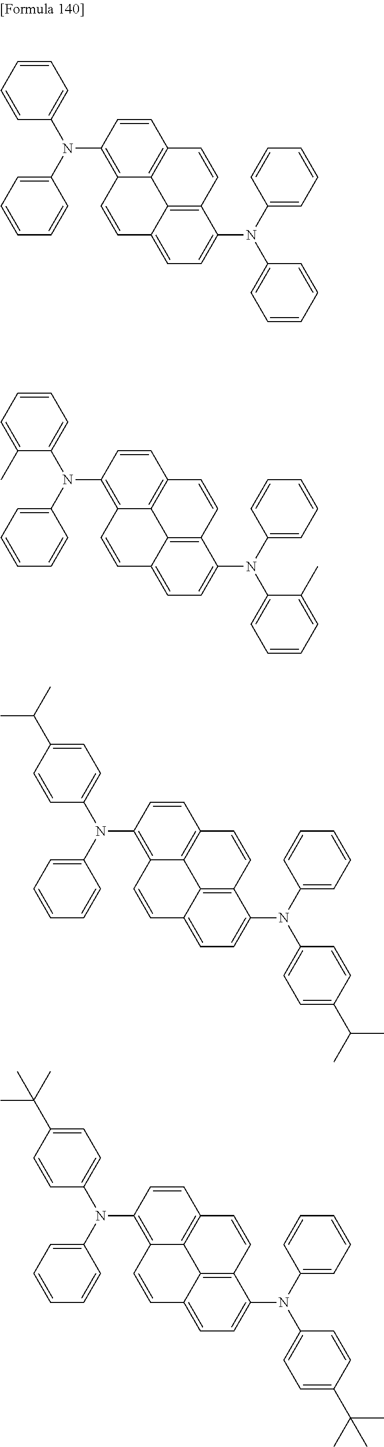

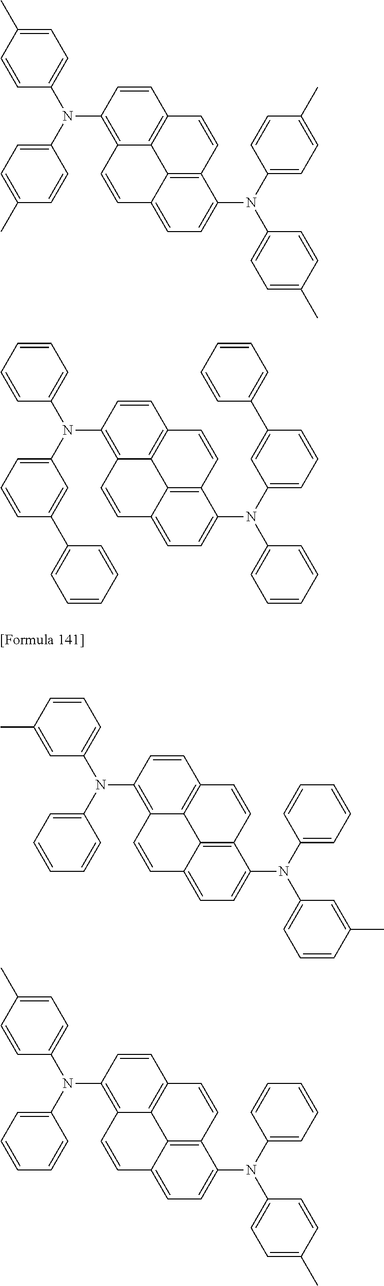

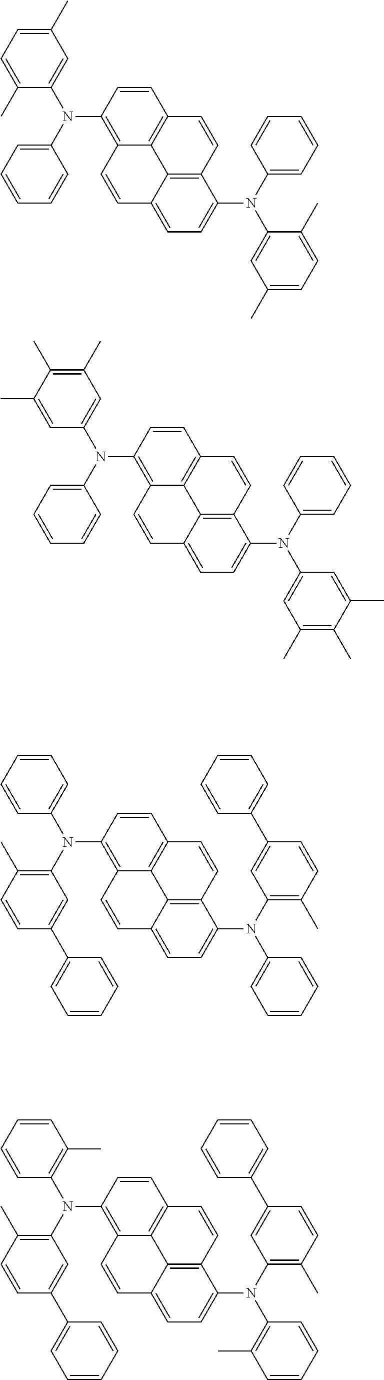

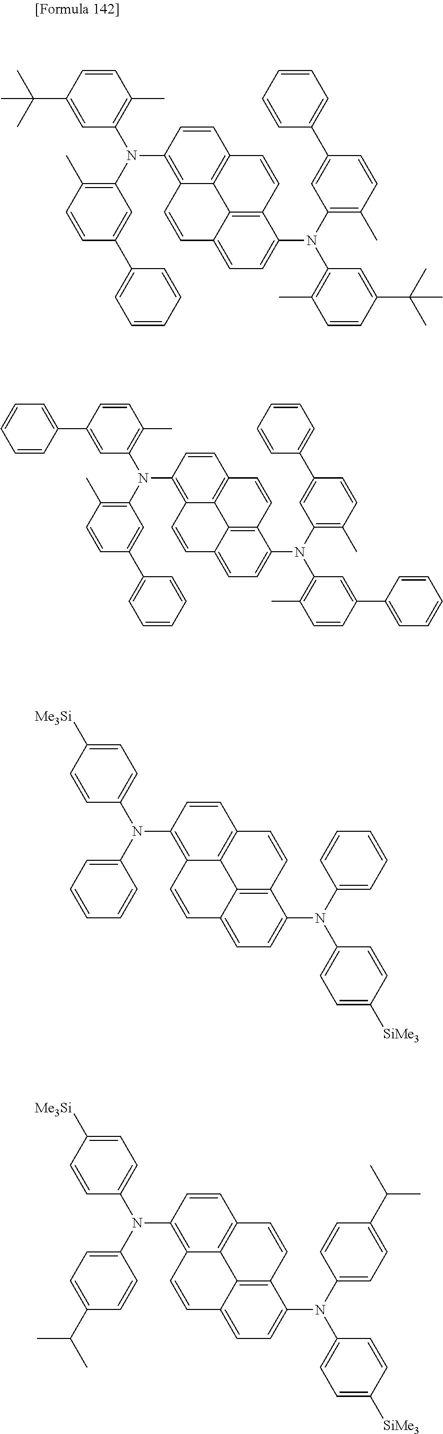

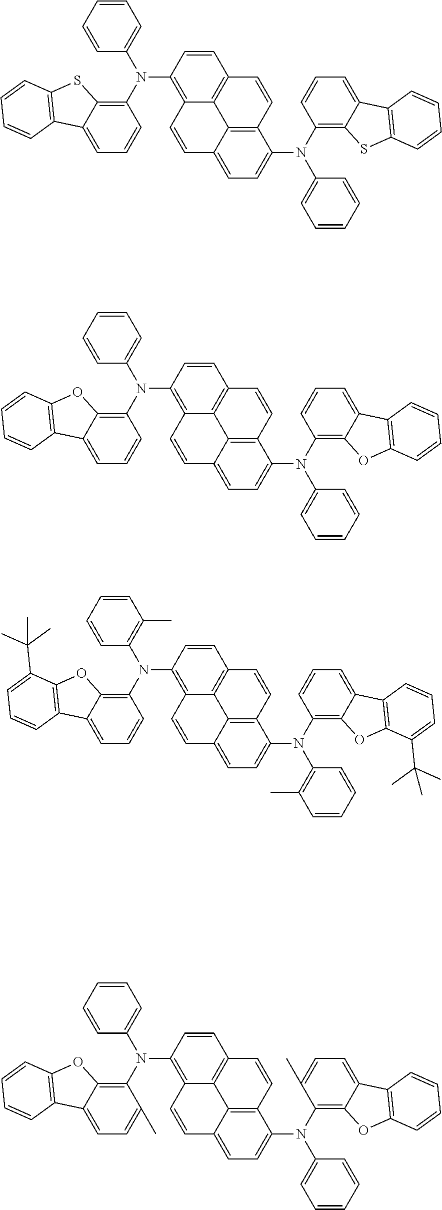 [SMILES: c1ccc(N(c2ccc3ccc4c(N(c5ccccc5)c5cccc6c5sc5ccccc56)ccc5ccc2c3c54)c2cccc3c2sc2ccccc23)cc1 ; c1ccc(N(c2ccc3ccc4c(N(c5ccccc5)c5cccc6c5oc5ccccc56)ccc5ccc2c3c54)c2cccc3c2oc2ccccc23)cc1 ; Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12 ; Cc1ccc2c(oc3ccccc32)c1N(c1ccccc1)c1ccc2ccc3c(N(c4ccccc4)c4c(C)ccc5c4oc4ccccc45)ccc4ccc1c2c43]

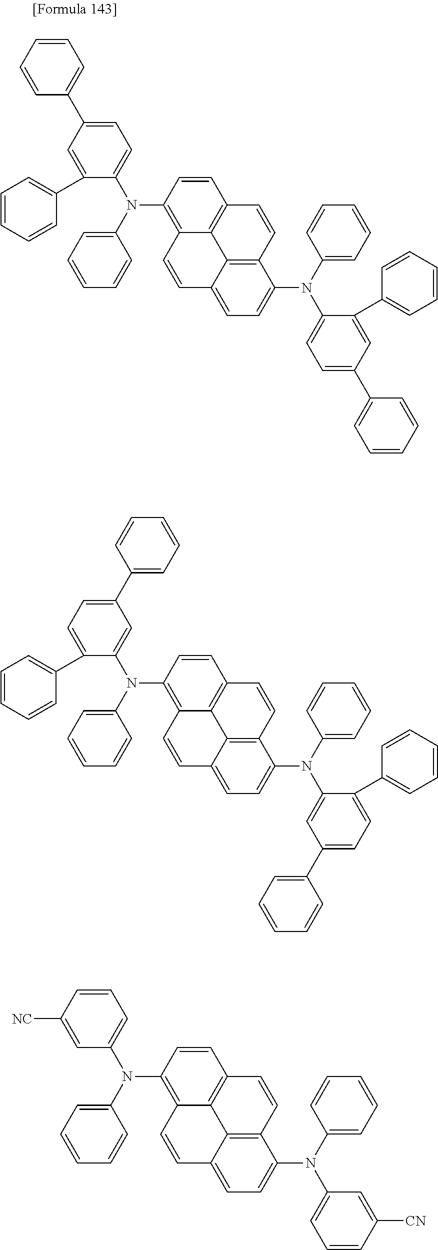 [SMILES: c1ccc(-c2ccc(N(c3ccccc3)c3ccc4ccc5c(N(c6ccccc6)c6ccc(-c7ccccc7)cc6-c6ccccc6)ccc6ccc3c4c65)c(-c3ccccc3)c2)cc1 ; c1ccc(-c2ccc(-c3ccccc3)c(N(c3ccccc3)c3ccc4ccc5c(N(c6ccccc6)c6cc(-c7ccccc7)ccc6-c6ccccc6)ccc6ccc3c4c65)c2)cc1 ; N#Cc1cccc(N(c2ccccc2)c2ccc3ccc4c(N(c5ccccc5)c5cccc(C#N)c5)ccc5ccc2c3c54)c1]

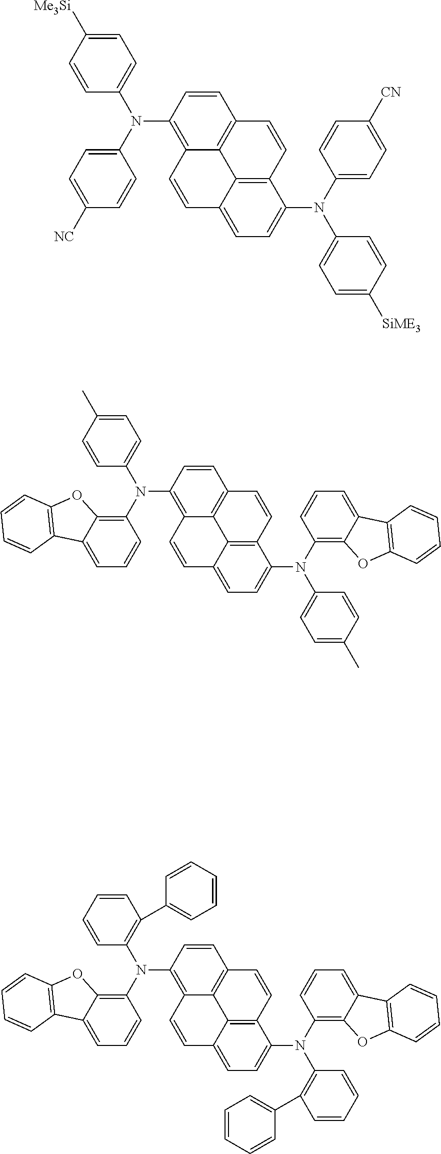

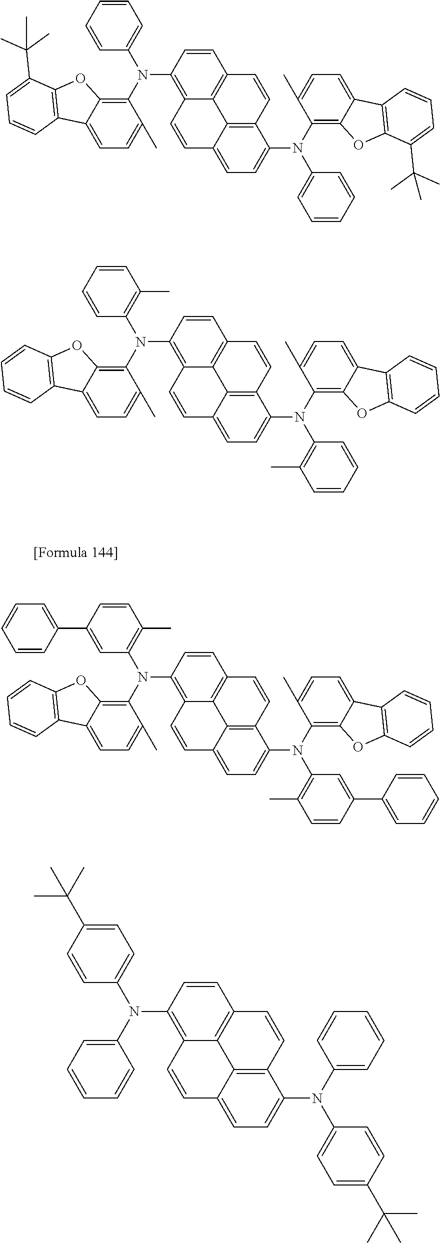

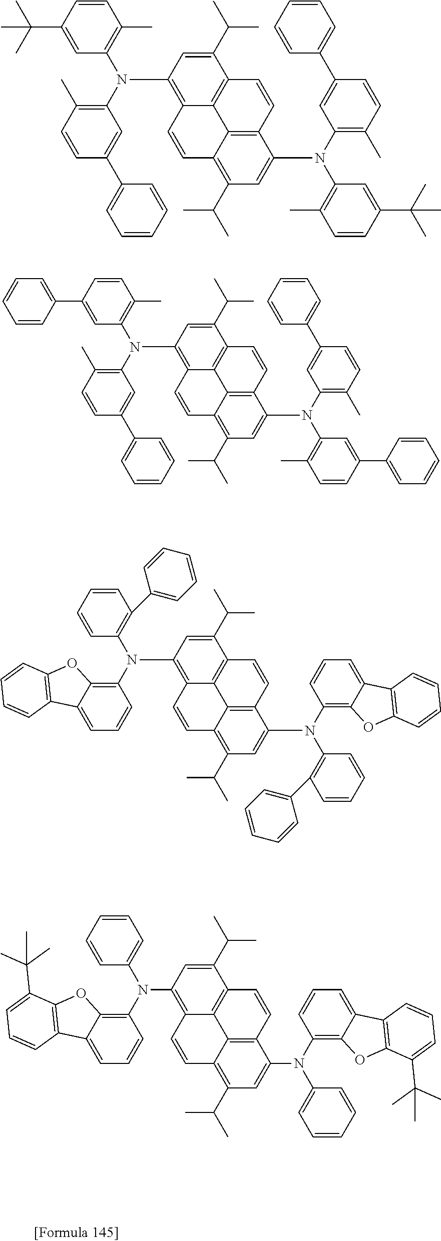

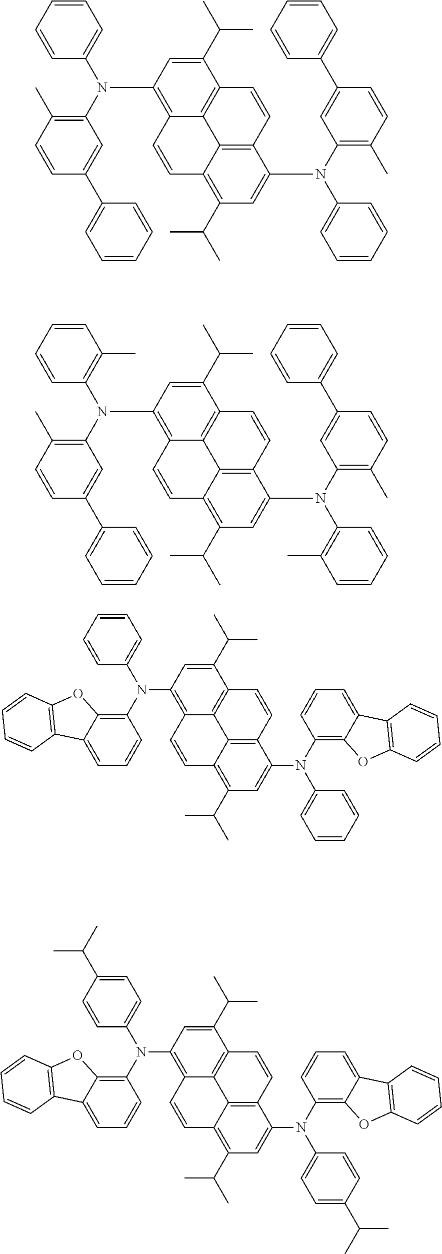

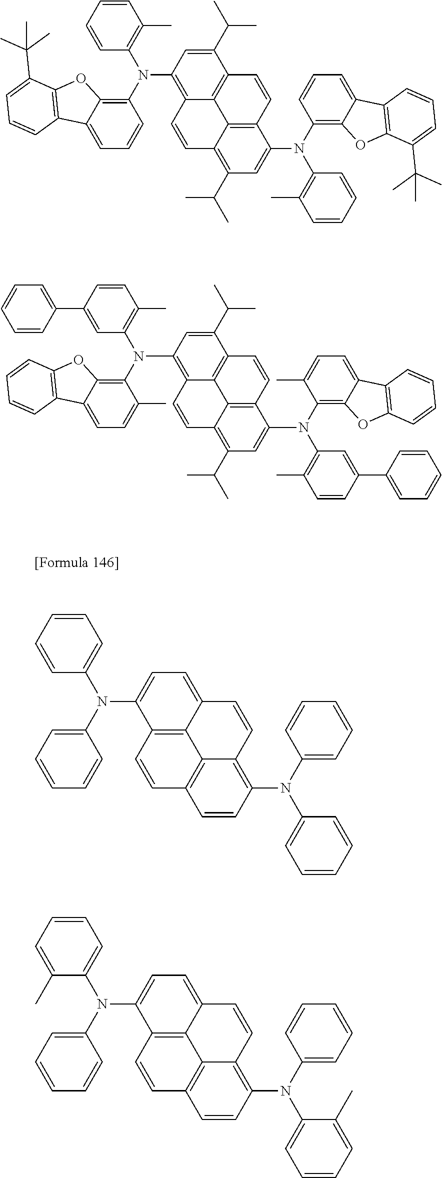 [SMILES: Cc1ccccc1N(c1cc(C(C)C)c2ccc3c(N(c4ccccc4C)c4cccc5c4oc4c(C(C)(C)C)cccc45)cc(C(C)C)c4ccc1c2c43)c1cccc2c1oc1c(C(C)(C)C)cccc12 ; Cc1ccc(-c2ccccc2)cc1N(c1cc(C(C)C)c2ccc3c(N(c4cc(-c5ccccc5)ccc4C)c4c(C)ccc5c4oc4ccccc45)cc(C(C)C)c4ccc1c2c43)c1c(C)ccc2c1oc1ccccc12 ; c1ccc(N(c2ccccc2)c2ccc3ccc4c(N(c5ccccc5)c5ccccc5)ccc5ccc2c3c54)cc1 ; Cc1ccccc1N(c1ccccc1)c1ccc2ccc3c(N(c4ccccc4)c4ccccc4C)ccc4ccc1c2c43]

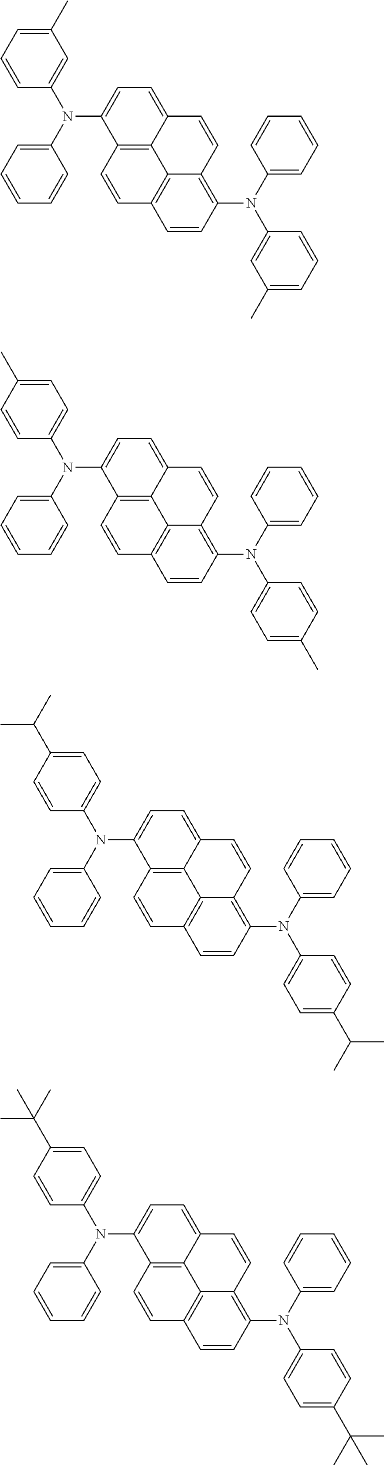

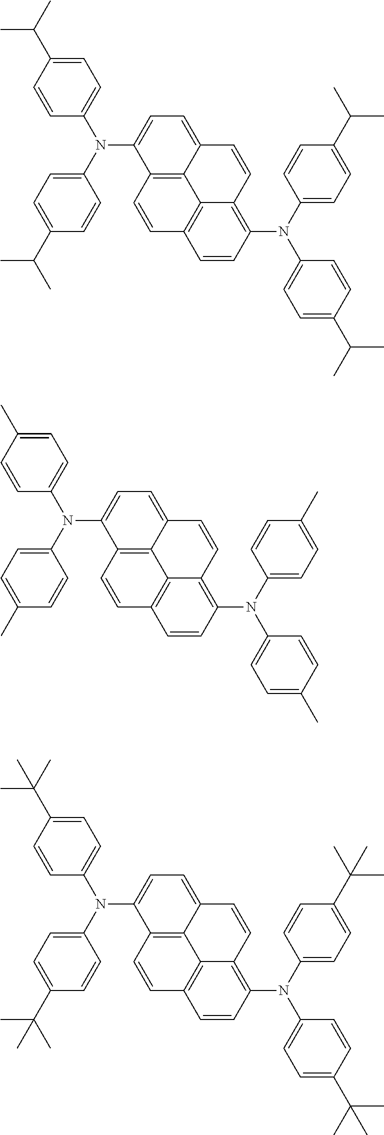

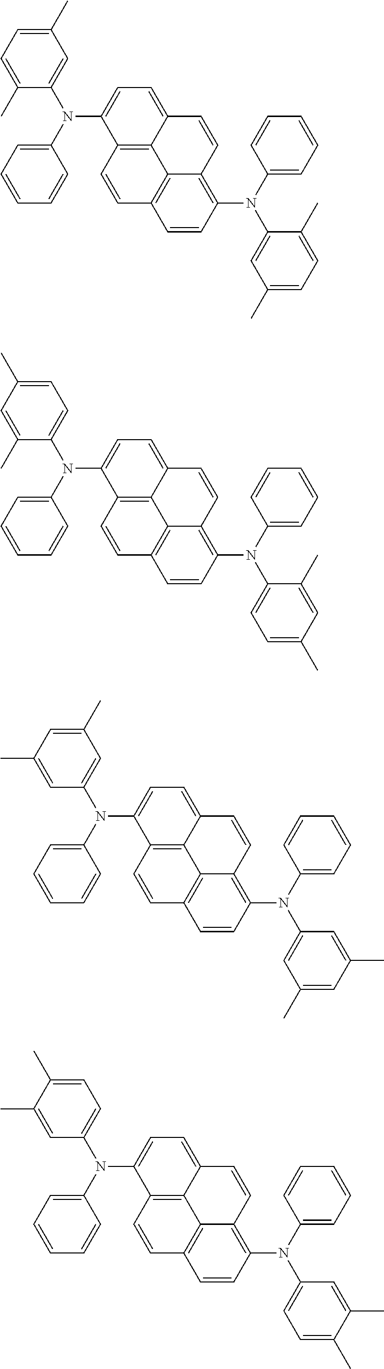

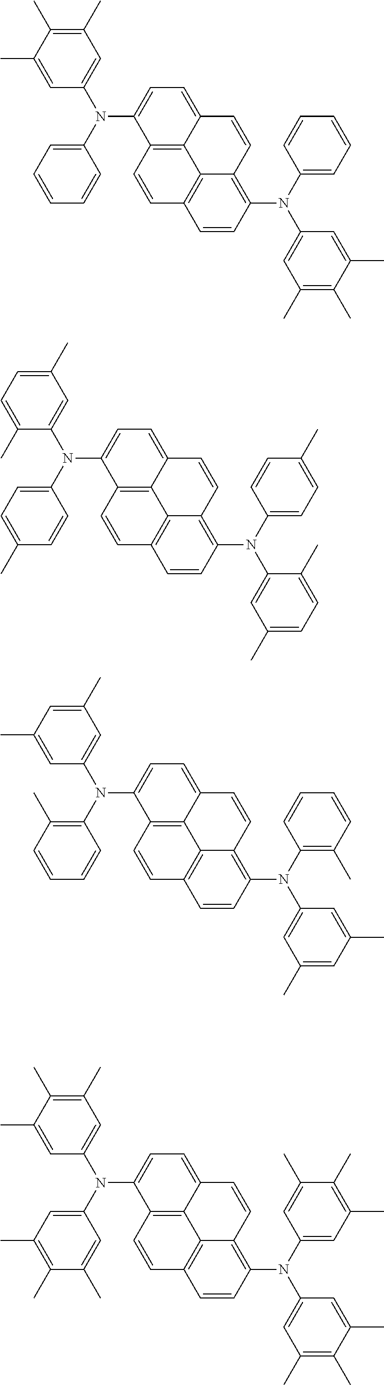

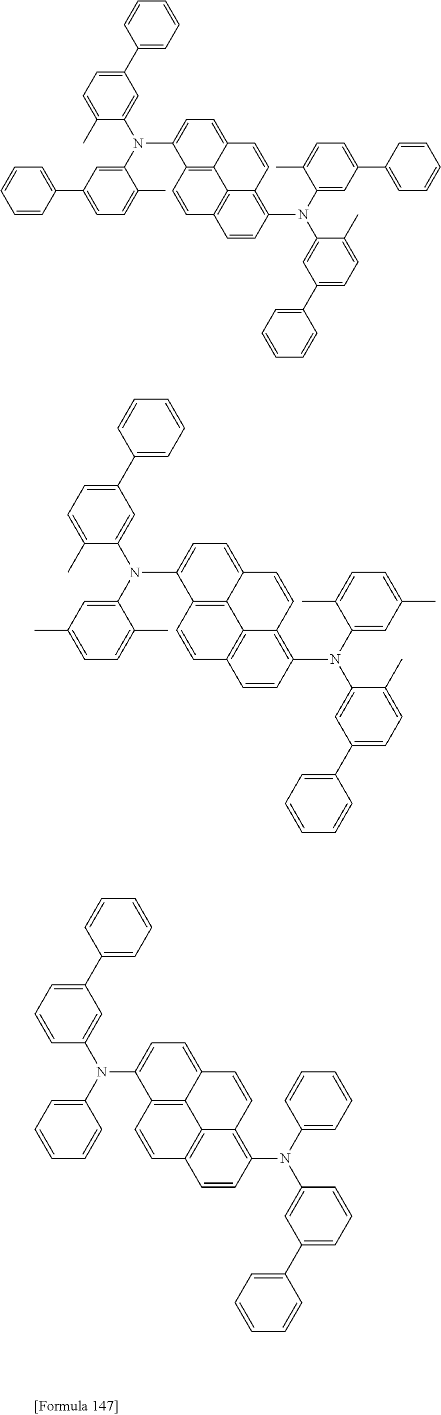

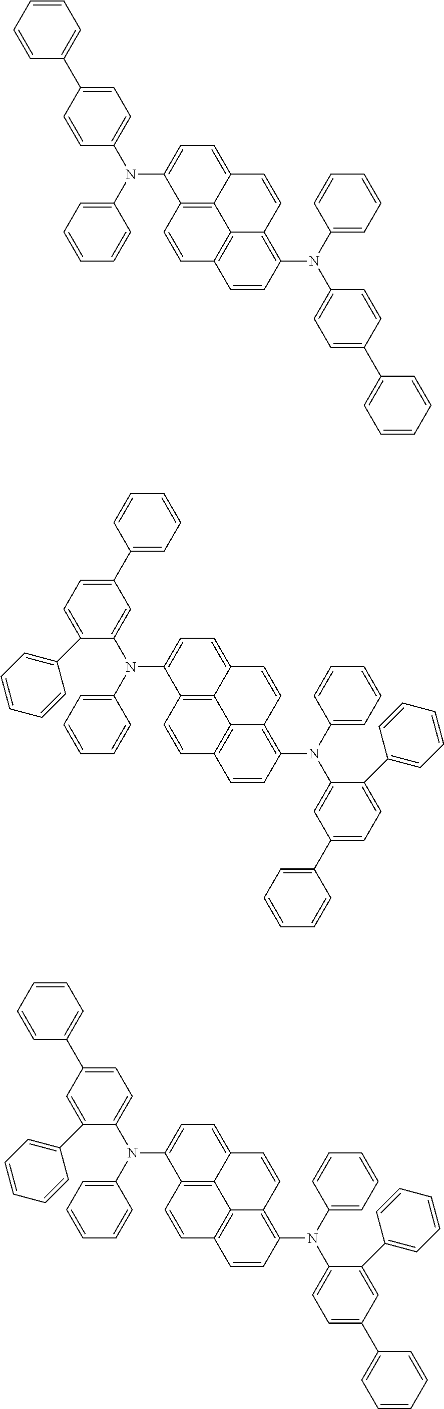 [SMILES: c1ccc(-c2ccc(N(c3ccccc3)c3ccc4ccc5c(N(c6ccccc6)c6ccc(-c7ccccc7)cc6)ccc6ccc3c4c65)cc2)cc1 ; c1ccc(-c2ccc(-c3ccccc3)c(N(c3ccccc3)c3ccc4ccc5c(N(c6ccccc6)c6cc(-c7ccccc7)ccc6-c6ccccc6)ccc6ccc3c4c65)c2)cc1 ; c1ccc(-c2ccc(N(c3ccccc3)c3ccc4ccc5c(N(c6ccccc6)c6ccc(-c7ccccc7)cc6-c6ccccc6)ccc6ccc3c4c65)c(-c3ccccc3)c2)cc1]

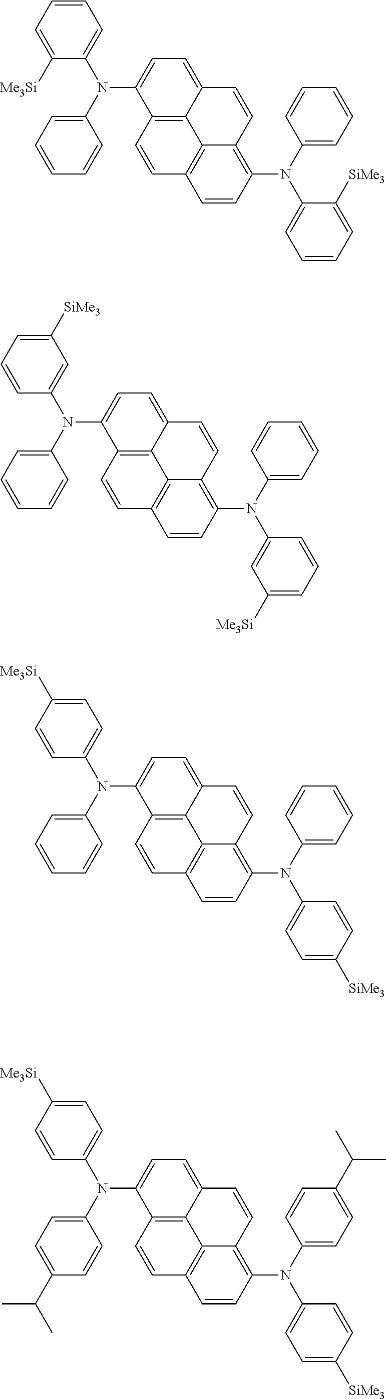 [SMILES: C[Si](C)(C)c1ccccc1N(c1ccccc1)c1ccc2ccc3c(N(c4ccccc4)c4ccccc4[Si](C)(C)C)ccc4ccc1c2c43 ; C[Si](C)(C)c1cccc(N(c2ccccc2)c2ccc3ccc4c(N(c5ccccc5)c5cccc([Si](C)(C)C)c5)ccc5ccc2c3c54)c1 ; C[Si](C)(C)c1ccc(N(c2ccccc2)c2ccc3ccc4c(N(c5ccccc5)c5ccc([Si](C)(C)C)cc5)ccc5ccc2c3c54)cc1 ; CC(C)c1ccc(N(c2ccc([Si](C)(C)C)cc2)c2ccc3ccc4c(N(c5ccc(C(C)C)cc5)c5ccc([Si](C)(C)C)cc5)ccc5ccc2c3c54)cc1]

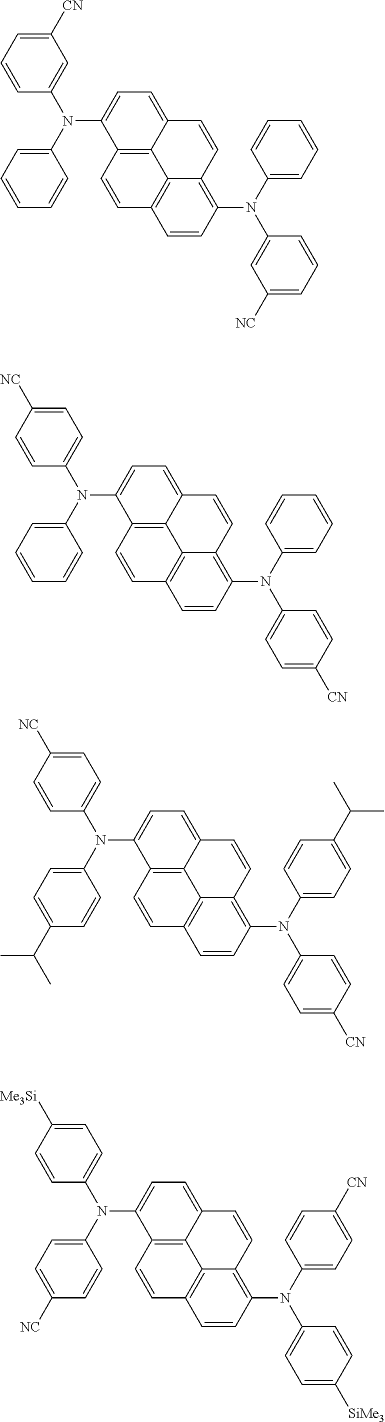 [SMILES: N#Cc1cccc(N(c2ccccc2)c2ccc3ccc4c(N(c5ccccc5)c5cccc(C#N)c5)ccc5ccc2c3c54)c1 ; N#Cc1ccc(N(c2ccccc2)c2ccc3ccc4c(N(c5ccccc5)c5ccc(C#N)cc5)ccc5ccc2c3c54)cc1 ; CC(C)c1ccc(N(c2ccc(C#N)cc2)c2ccc3ccc4c(N(c5ccc(C#N)cc5)c5ccc(C(C)C)cc5)ccc5ccc2c3c54)cc1 ; C[Si](C)(C)c1ccc(N(c2ccc(C#N)cc2)c2ccc3ccc4c(N(c5ccc(C#N)cc5)c5ccc([Si](C)(C)C)cc5)ccc5ccc2c3c54)cc1]

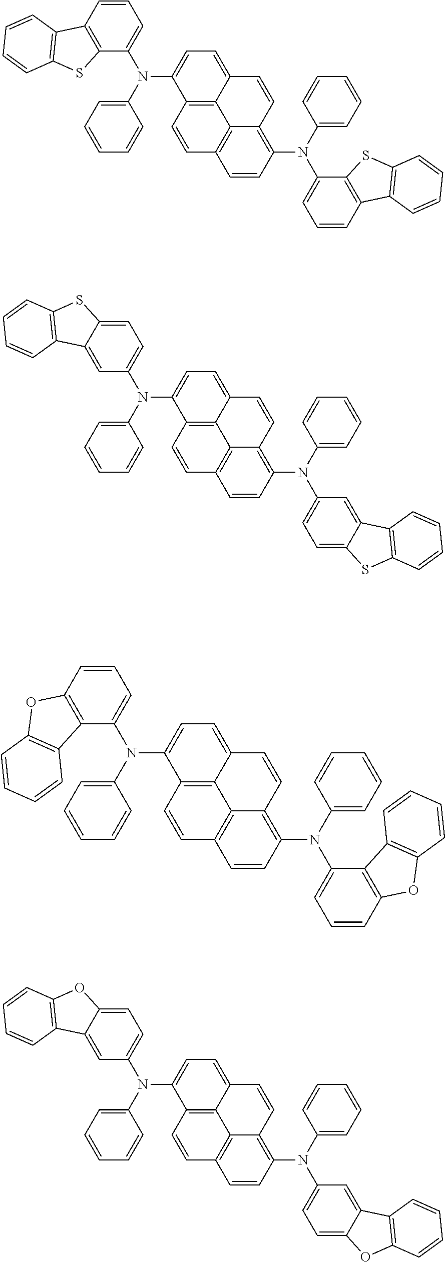 [SMILES: c1ccc(N(c2ccc3ccc4c(N(c5ccccc5)c5cccc6c5sc5ccccc56)ccc5ccc2c3c54)c2cccc3c2sc2ccccc23)cc1 ; c1ccc(N(c2ccc3sc4ccccc4c3c2)c2ccc3ccc4c(N(c5ccccc5)c5ccc6sc7ccccc7c6c5)ccc5ccc2c3c54)cc1 ; c1ccc(N(c2ccc3ccc4c(N(c5ccccc5)c5cccc6oc7ccccc7c56)ccc5ccc2c3c54)c2cccc3oc4ccccc4c23)cc1 ; c1ccc(N(c2ccc3oc4ccccc4c3c2)c2ccc3ccc4c(N(c5ccccc5)c5ccc6oc7ccccc7c6c5)ccc5ccc2c3c54)cc1]

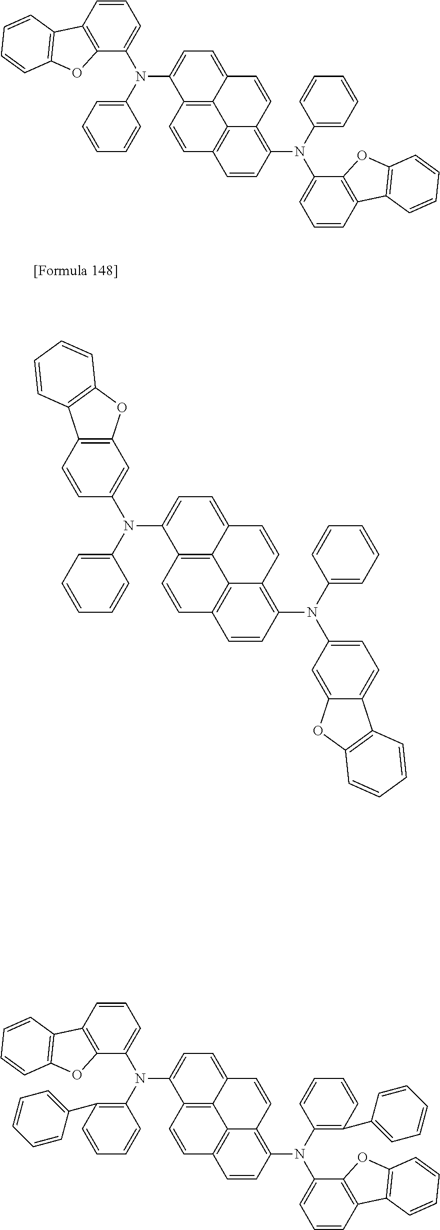 [SMILES: c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3c(c2)oc2ccccc23)c2ccc3ccc4c(N(c5ccccc5)c5ccc6c(c5)oc5ccccc56)ccc5ccc2c3c54)cc1 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1]

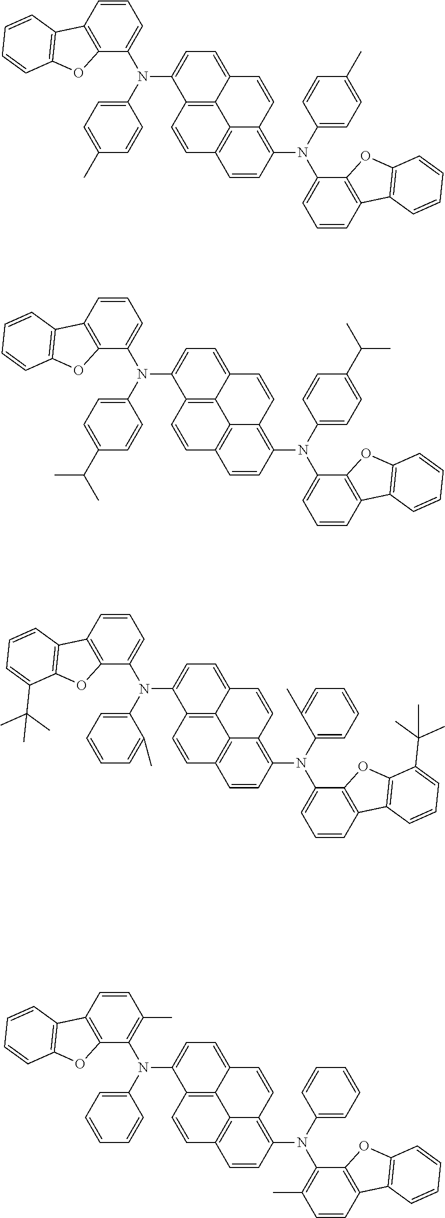 [SMILES: Cc1ccc(N(c2ccc3ccc4c(N(c5ccc(C)cc5)c5cccc6c5oc5ccccc56)ccc5ccc2c3c54)c2cccc3c2oc2ccccc23)cc1 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12 ; Cc1ccc2c(oc3ccccc32)c1N(c1ccccc1)c1ccc2ccc3c(N(c4ccccc4)c4c(C)ccc5c4oc4ccccc45)ccc4ccc1c2c43]

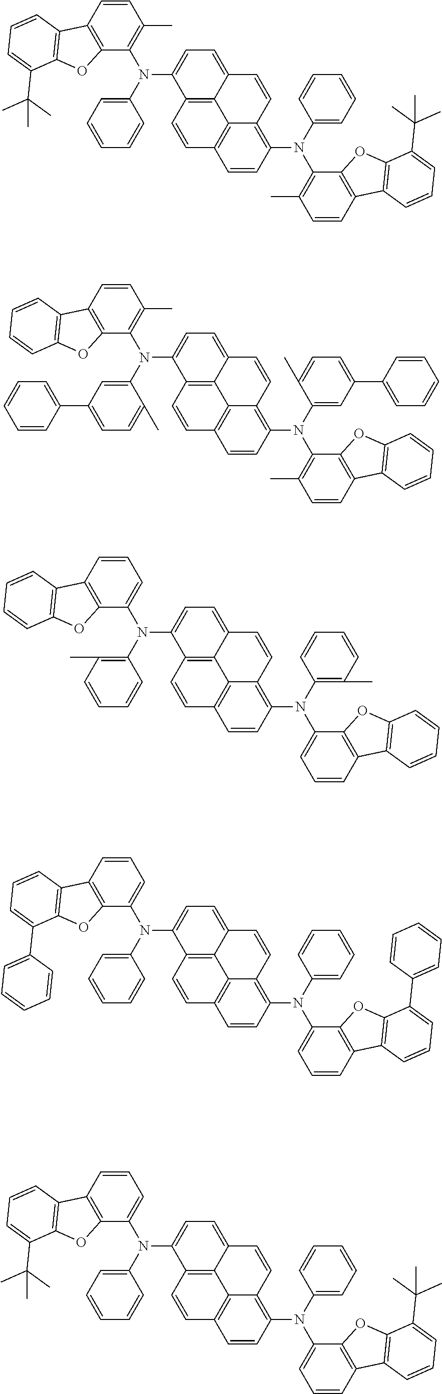

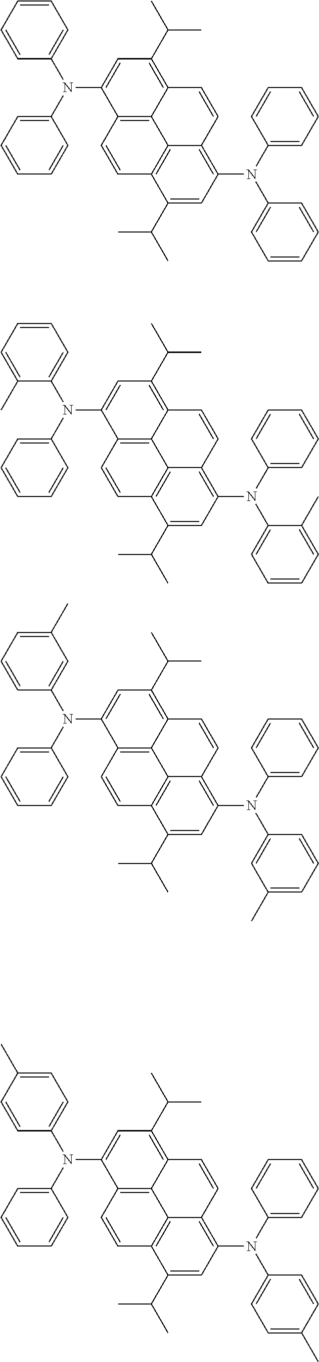 [SMILES: CC(C)c1cc(N(c2ccccc2)c2ccccc2)c2ccc3c(C(C)C)cc(N(c4ccccc4)c4ccccc4)c4ccc1c2c34 ; Cc1ccccc1N(c1ccccc1)c1cc(C(C)C)c2ccc3c(N(c4ccccc4)c4ccccc4C)cc(C(C)C)c4ccc1c2c43 ; Cc1cccc(N(c2ccccc2)c2cc(C(C)C)c3ccc4c(N(c5ccccc5)c5cccc(C)c5)cc(C(C)C)c5ccc2c3c54)c1 ; Cc1ccc(N(c2ccccc2)c2cc(C(C)C)c3ccc4c(N(c5ccccc5)c5ccc(C)cc5)cc(C(C)C)c5ccc2c3c54)cc1]

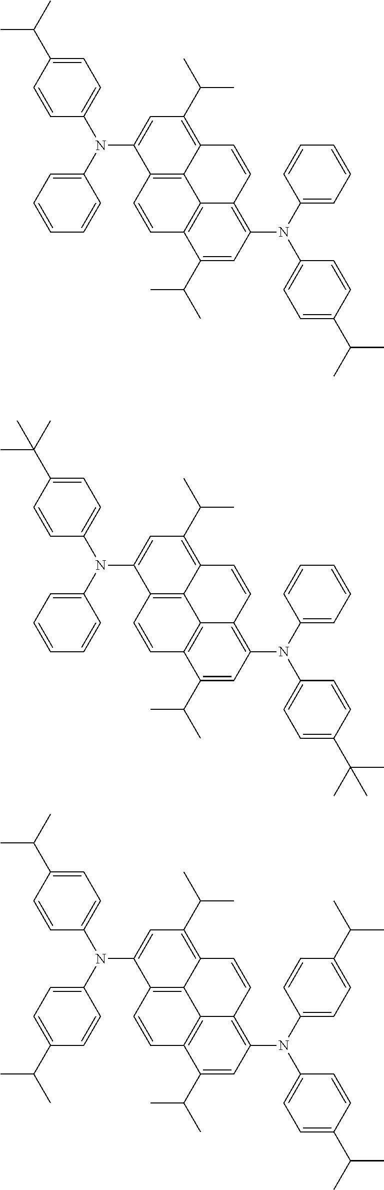

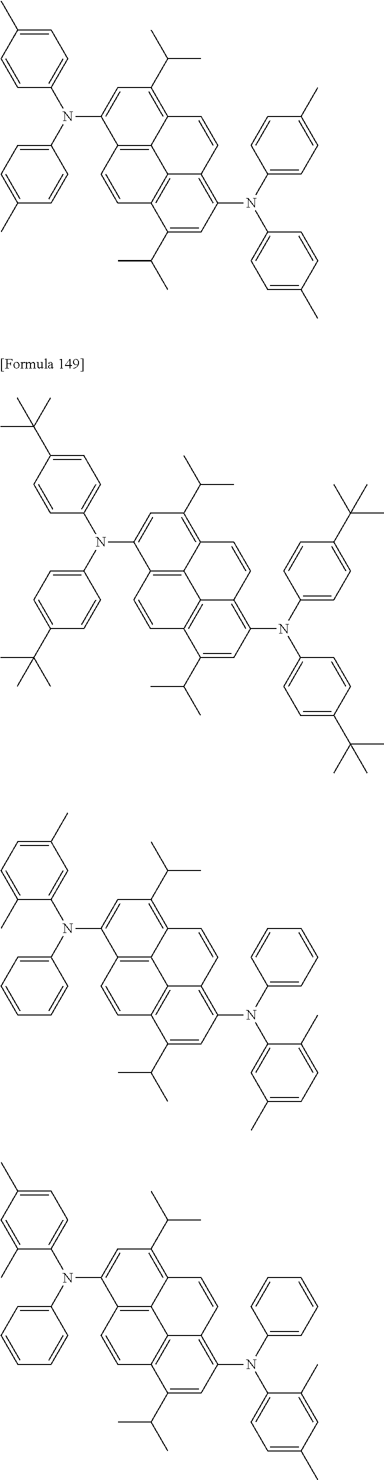

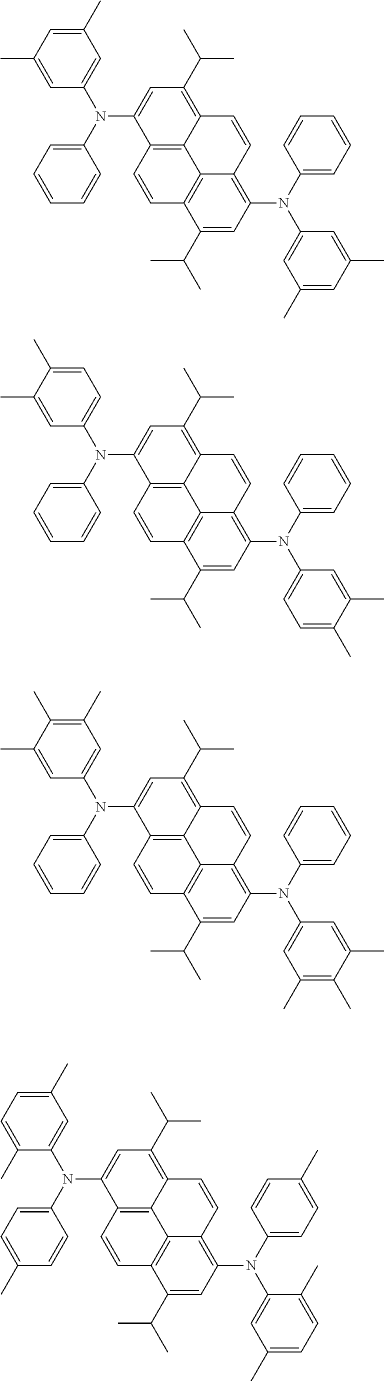

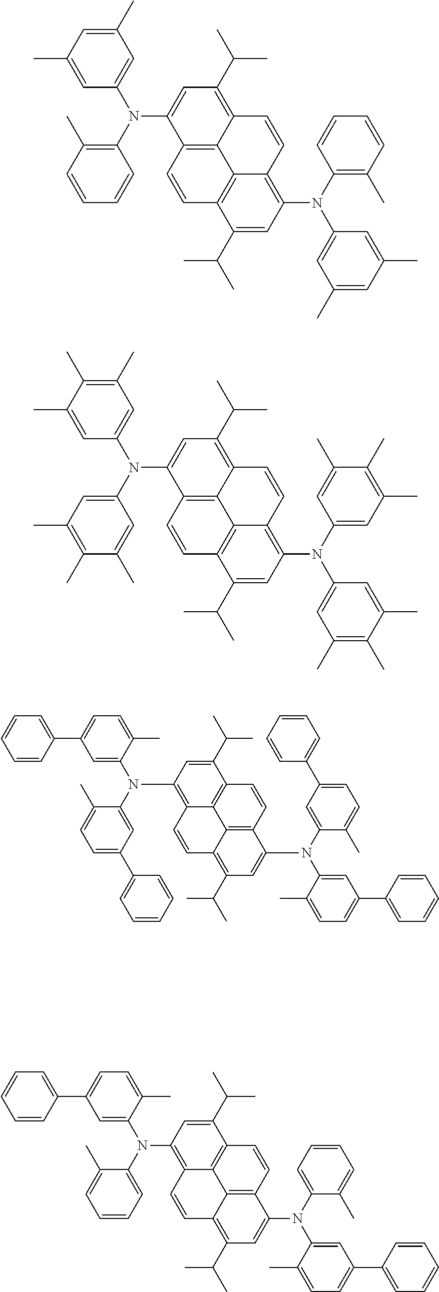

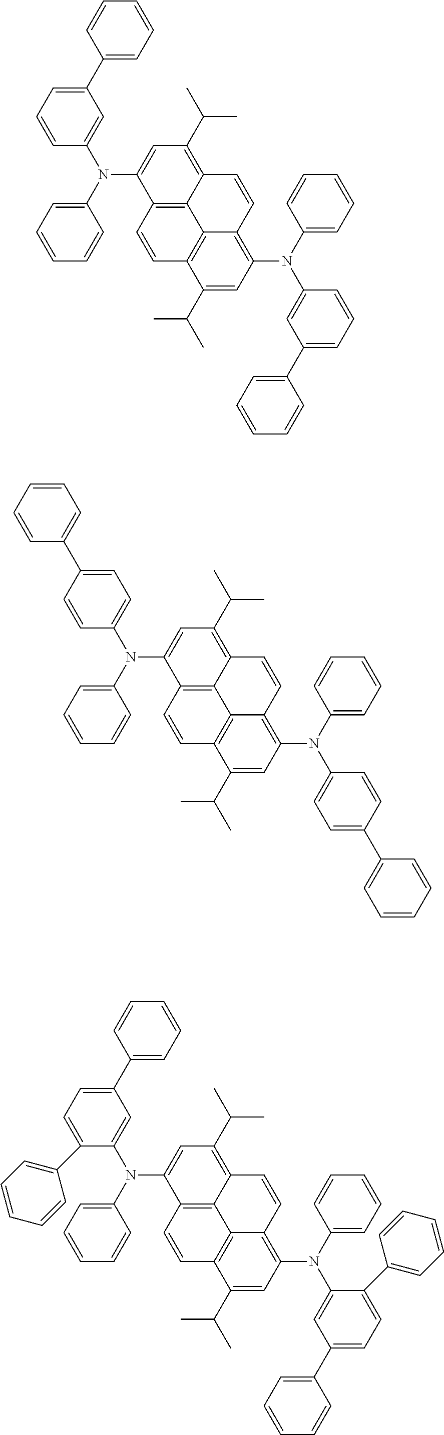 [SMILES: CC(C)c1cc(N(c2ccccc2)c2cccc(-c3ccccc3)c2)c2ccc3c(C(C)C)cc(N(c4ccccc4)c4cccc(-c5ccccc5)c4)c4ccc1c2c34 ; CC(C)c1cc(N(c2ccccc2)c2ccc(-c3ccccc3)cc2)c2ccc3c(C(C)C)cc(N(c4ccccc4)c4ccc(-c5ccccc5)cc4)c4ccc1c2c34 ; CC(C)c1cc(N(c2ccccc2)c2cc(-c3ccccc3)ccc2-c2ccccc2)c2ccc3c(C(C)C)cc(N(c4ccccc4)c4cc(-c5ccccc5)ccc4-c4ccccc4)c4ccc1c2c34]

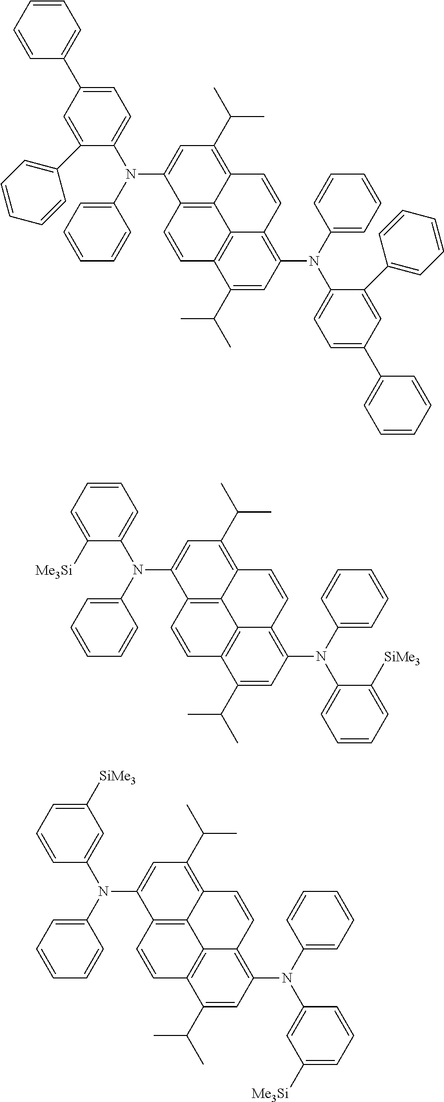 [SMILES: CC(C)c1cc(N(c2ccccc2)c2ccc(-c3ccccc3)cc2-c2ccccc2)c2ccc3c(C(C)C)cc(N(c4ccccc4)c4ccc(-c5ccccc5)cc4-c4ccccc4)c4ccc1c2c34 ; CC(C)c1cc(N(c2ccccc2)c2ccccc2[Si](C)(C)C)c2ccc3c(C(C)C)cc(N(c4ccccc4)c4ccccc4[Si](C)(C)C)c4ccc1c2c34 ; CC(C)c1cc(N(c2ccccc2)c2cccc([Si](C)(C)C)c2)c2ccc3c(C(C)C)cc(N(c4ccccc4)c4cccc([Si](C)(C)C)c4)c4ccc1c2c34]

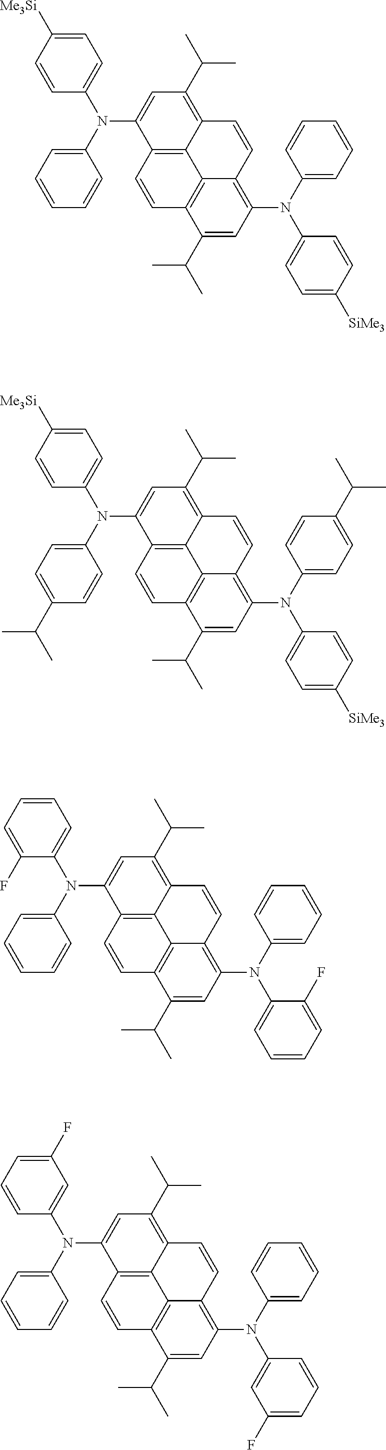 [SMILES: CC(C)c1cc(N(c2ccccc2)c2ccc([Si](C)(C)C)cc2)c2ccc3c(C(C)C)cc(N(c4ccccc4)c4ccc([Si](C)(C)C)cc4)c4ccc1c2c34 ; CC(C)c1ccc(N(c2ccc([Si](C)(C)C)cc2)c2cc(C(C)C)c3ccc4c(N(c5ccc(C(C)C)cc5)c5ccc([Si](C)(C)C)cc5)cc(C(C)C)c5ccc2c3c54)cc1 ; CC(C)c1cc(N(c2ccccc2)c2ccccc2F)c2ccc3c(C(C)C)cc(N(c4ccccc4)c4ccccc4F)c4ccc1c2c34 ; CC(C)c1cc(N(c2ccccc2)c2cccc(F)c2)c2ccc3c(C(C)C)cc(N(c4ccccc4)c4cccc(F)c4)c4ccc1c2c34]

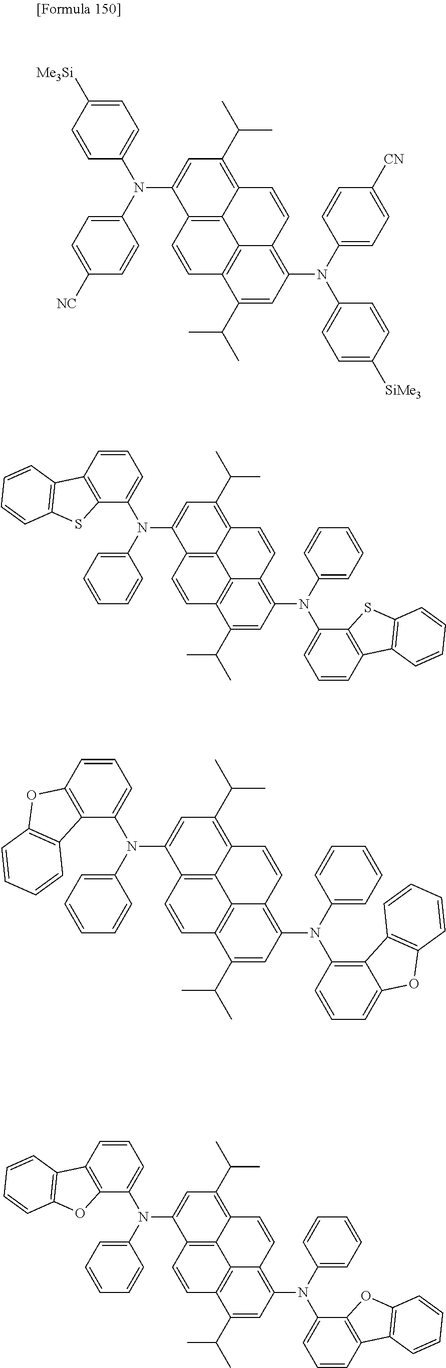 [SMILES: CC(C)c1cc(N(c2ccc(C#N)cc2)c2ccc([Si](C)(C)C)cc2)c2ccc3c(C(C)C)cc(N(c4ccc(C#N)cc4)c4ccc([Si](C)(C)C)cc4)c4ccc1c2c34 ; CC(C)c1cc(N(c2ccccc2)c2cccc3c2sc2ccccc23)c2ccc3c(C(C)C)cc(N(c4ccccc4)c4cccc5c4sc4ccccc45)c4ccc1c2c34 ; CC(C)c1cc(N(c2ccccc2)c2cccc3oc4ccccc4c23)c2ccc3c(C(C)C)cc(N(c4ccccc4)c4cccc5oc6ccccc6c45)c4ccc1c2c34 ; CC(C)c1cc(N(c2ccccc2)c2cccc3c2oc2ccccc23)c2ccc3c(C(C)C)cc(N(c4ccccc4)c4cccc5c4oc4ccccc45)c4ccc1c2c34]

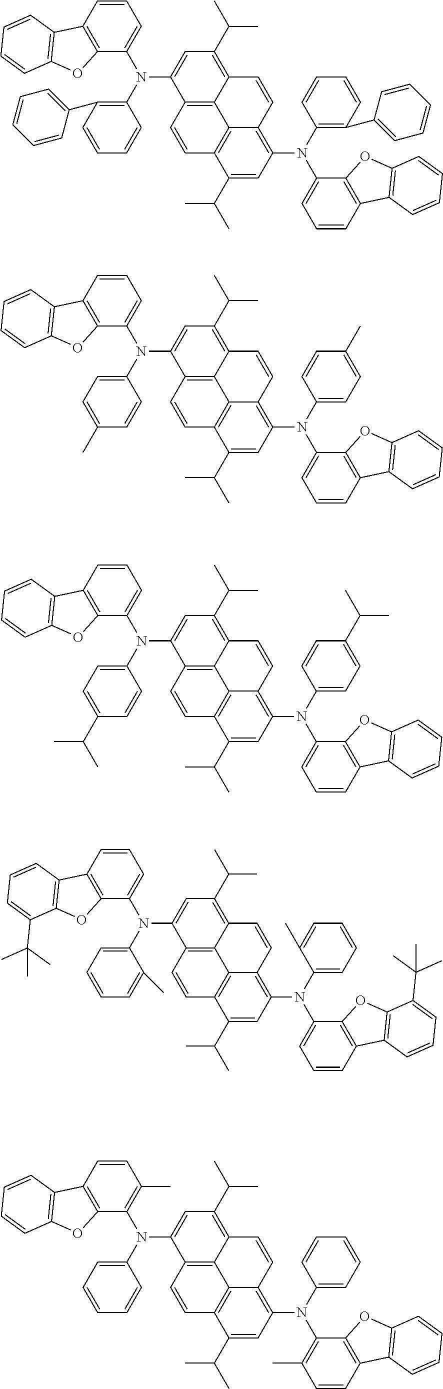

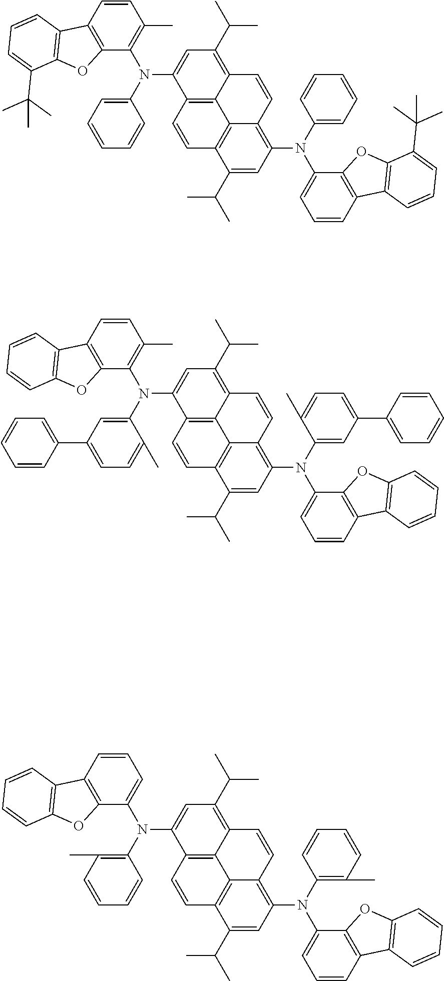 [SMILES: Cc1ccc2c(oc3c(C(C)(C)C)cccc32)c1N(c1ccccc1)c1cc(C(C)C)c2ccc3c(N(c4ccccc4)c4cccc5c4oc4c(C(C)(C)C)cccc45)cc(C(C)C)c4ccc1c2c43 ; Cc1ccc(-c2ccccc2)cc1N(c1cc(C(C)C)c2ccc3c(N(c4cc(-c5ccccc5)ccc4C)c4c(C)ccc5c4oc4ccccc45)cc(C(C)C)c4ccc1c2c43)c1cccc2c1oc1ccccc12 ; Cc1ccccc1N(c1cc(C(C)C)c2ccc3c(N(c4ccccc4C)c4cccc5c4oc4ccccc45)cc(C(C)C)c4ccc1c2c43)c1cccc2c1oc1ccccc12]

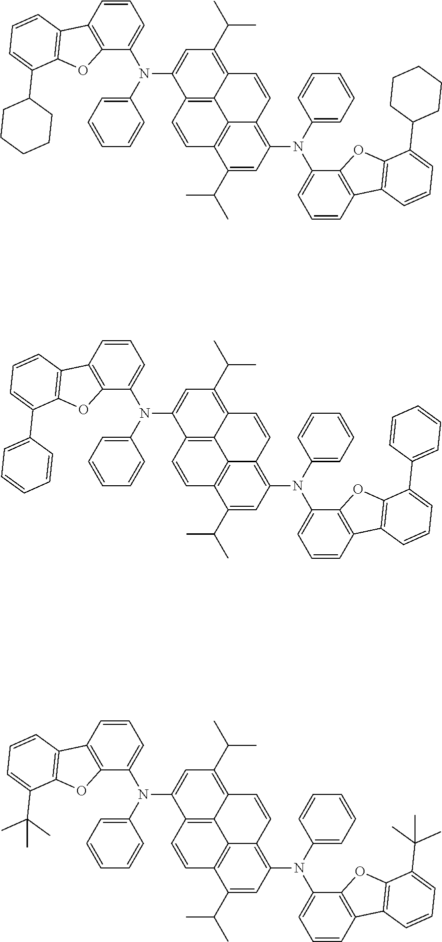 [SMILES: CC(C)c1cc(N(c2ccccc2)c2cccc3c2oc2c(C4CCCCC4)cccc23)c2ccc3c(C(C)C)cc(N(c4ccccc4)c4cccc5c4oc4c(C6CCCCC6)cccc45)c4ccc1c2c34 ; CC(C)c1cc(N(c2ccccc2)c2cccc3c2oc2c(-c4ccccc4)cccc23)c2ccc3c(C(C)C)cc(N(c4ccccc4)c4cccc5c4oc4c(-c6ccccc6)cccc45)c4ccc1c2c34 ; CC(C)c1cc(N(c2ccccc2)c2cccc3c2oc2c(C(C)(C)C)cccc23)c2ccc3c(C(C)C)cc(N(c4ccccc4)c4cccc5c4oc4c(C(C)(C)C)cccc45)c4ccc1c2c34]

Compound Represented by Formula (4)

A compound represented by the formula (4) is described below.

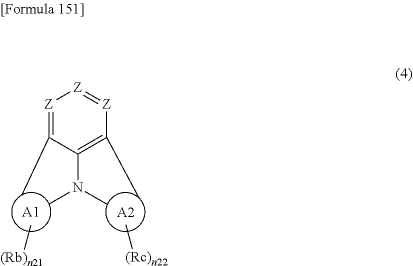

In the formula (4),
Z each independently represent CRa or a nitrogen atom, the A1 ring and the A2 ring each independently represent a substituted or unsubstituted aromatic hydrocarbon ring having 6 to 50 ring carbon atoms, or a substituted or unsubstituted heterocycle having 5 to 50 ring atoms, and
when a plurality of Ra are present, at least one combination of adjacent two or more of the plurality of Ra are mutually bonded to form a substituted or unsubstituted monocyclic ring, are mutually bonded to form a substituted or unsubstituted fused ring, or are not mutually bonded,
n21 and n22 each independently represent 0, 1, 2, 3, or 4,
when a plurality of Rb are present, at least one combination of adjacent two or more of the plurality of Rb are mutually bonded to form a substituted or unsubstituted monocyclic ring, are mutually bonded to form a substituted or unsubstituted fused ring, or are not mutually bonded,
when a plurality of Rc are present, at least one combination of adjacent two or more of the plurality of Rc are mutually bonded to form a substituted or unsubstituted monocyclic ring, are mutually bonded to form a substituted or unsubstituted fused ring, or are not mutually bonded, and
Ra, Rb, and Rc that do not form the monocyclic ring and do not form the fused ring each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, cyano group, a nitro group, a substituted or unsubstituted aryl group having 6 to 50 ring carbon atoms, or a substituted or unsubstituted heterocyclic group having 5 to 50 ring atoms.

The “aromatic hydrocarbon rings” of the A1 ring and the A2 ring have the same structure as compounds in which a hydrogen atom is introduced into the “aryl group” described above.

The “aromatic hydrocarbon rings” of the A1 ring and the A2 ring have two carbon atoms on the central fused bicyclic structure of the formula (4) as ring atoms.

Specific examples of the “substituted or unsubstituted aromatic hydrocarbon ring having 6 to 50 ring carbon atoms” include compounds in which a hydrogen atom is introduced into the “aryl group” described in the specific example group G1.

The “heterocycles” of the A1 ring and the A2 ring have the same structure as compounds in which a hydrogen atom is introduced into the “heterocyclic group” described above.

The “heterocycles” of the A1 ring and the A2 ring have two carbon atoms on the central fused two-ring structure of the formula (4) as ring atoms.

Specific examples of the “substituted or unsubstituted heterocycle having 5 to 50 ring atoms” include compounds in which a hydrogen atom is introduced into the “heterocyclic groups” described in the specific example group G2.

Rb is bonded to one of the carbon atoms forming the aromatic hydrocarbon ring of the A1 ring or to one of the atoms forming the heterocycle of the A1 ring.

Rc is bonded to one of the carbon atoms forming the aromatic hydrocarbon ring of the A2 ring or to one of the atoms forming the heterocycle of the A2 ring.

At least one of Ra, Rb, or Rc is preferably a group represented by the following formula (4a), and at least two of Ra, Rb, or Rc are more preferably groups represented by the following formula (4a).

[Formula 152]

*-L₄₀₁-Ar₄₀₁ (4a)

In the formula (4a),
L₄₀₁ represents a single bond, a substituted or unsubstituted arylene group having 6 to 30 ring carbon atoms, or a substituted or unsubstituted divalent heterocyclic group having 5 to 30 ring atoms, and
Ar₄₀₁ represents a substituted or unsubstituted aryl group having 6 to 50 ring carbon atoms, a substituted or unsubstituted heterocyclic group having 5 to 50 ring atoms, or a group represented by the following formula (4b).

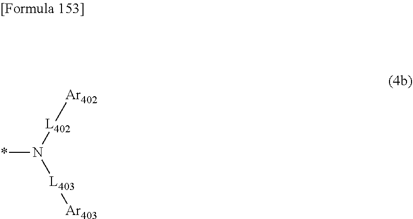

In the formula (4b),
L₄₀₂ and L₄₀₃ each independently represent a single bond, a substituted or unsubstituted arylene group having 6 to 30 ring carbon atoms, or a substituted or unsubstituted divalent heterocyclic group having 5 to 30 ring atoms,
a pair of Ar₄₀₂ and Ar₄₀₃ are mutually bonded to form a substituted or unsubstituted monocyclic ring, are mutually bonded to form a substituted or unsubstituted fused ring, or are not mutually bonded, and
Ar₄₀₂ and Ar₄₀₃ that do not form the monocyclic ring and do not form the fused ring each independently represent a substituted or unsubstituted aryl group having 6 to 50 ring carbon atoms, or a substituted or unsubstituted heterocyclic group having 5 to 50 ring atoms.

In an exemplary embodiment, a compound represented by the formula (4) is represented by the following formula (42).

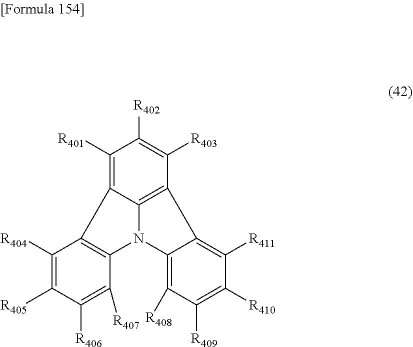

In the formula (42),
at least one combination of adjacent two or more of R₄₀₁ to R₄₁₁ are mutually bonded to form a substituted or unsubstituted monocyclic ring, are mutually bonded to form a substituted or unsubstituted fused ring, or are not mutually bonded, and
R₄₀₁ to R₄₁₁ that do not form the monocyclic ring and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of R₄₀₁ to R₄₁₁ is preferably a group represented by the formula (4a), and at least two of Ra, Rb, or Rc are more preferably groups represented by the formula (4a).

R₄₀₄ and R₄₁₁ are preferably groups represented by the formula (4a).

In an exemplary embodiment, a compound represented by the formula (4) is a compound in which a structure represented by the formula (4-1) or the formula (4-2) described below is bonded to the A1 ring.

In an exemplary embodiment, a compound represented by the formula (42) is a compound in which a structure represented by the formula (4-1) or the formula (4-2) described below is bonded to the ring bonded to R₄₀₄ to R₄₀₇.

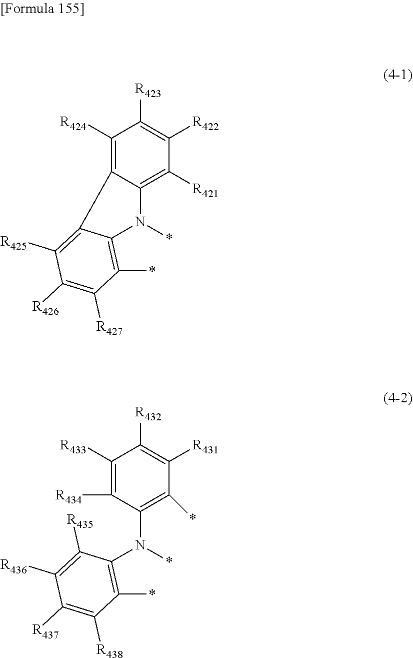

Two * in the formula (4-1) are each independently bonded to a ring carbon atom of the aromatic hydrocarbon ring or a ring atom of the heterocycle of the A1 ring in the formula (4), or to one of R₄₀₄ to R₄₀₇ in the formula (42),
three * in the formula (4-2) are each independently bonded to a ring carbon atom of the aromatic hydrocarbon ring or a ring atom of the heterocycle of the A1 ring in the formula (4), or to one of R₄₀₄ to R₄₀₇ in the formula (42),
at least one combination of adjacent two or more of R₄₂₁ to R₄₂₇ are mutually bonded to form a substituted or unsubstituted monocyclic ring, are mutually bonded to form a substituted or unsubstituted fused ring, or are not mutually bonded,
at least one combination of adjacent two or more of R₄₃₁ to R₄₃₈ are mutually bonded to form a substituted or unsubstituted monocyclic ring, are mutually bonded to form a substituted or unsubstituted fused ring, or are not mutually bonded, and
R₄₂₁ to R₄₂₇ and R₄₃₁ to R₄₃₈ that do not form the monocyclic ring and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, a compound represented by the formula (4) is a compound represented by the following formula (41-3), (41-4), or (41-5).

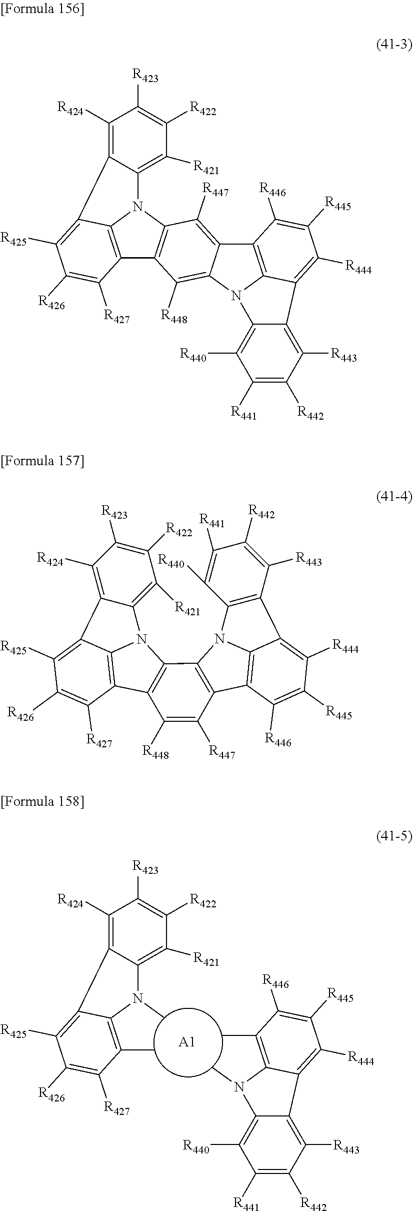

In the formulae (41-3), (41-4), and (41-5),
the A1 ring is as defined in the formula (4),
R₄₂₁ to R₄₂₇ represent the same as R₄₂₁ to R₄₂₇, respectively, in the formula (4-1), and
R₄₄₀ to R₄₄₈ each independently represent the same as R₄₀₁ to R₄₁₁ in the formula (42).

In an exemplary embodiment, a substituted or unsubstituted aromatic hydrocarbon ring having 6 to 50 ring carbon atoms of the A1 ring in the formula (41-5) is a substituted or unsubstituted naphthalene ring, or a substituted or unsubstituted fluorene ring.

In an exemplary embodiment, a substituted or unsubstituted heterocycle having 5 to 50 ring atoms of the A1 ring in the formula (41-5) is a substituted or unsubstituted dibenzofuran ring, a substituted or unsubstituted carbazole ring, or a substituted or unsubstituted dibenzothiophene ring.

In an exemplary embodiment, a compound represented by the formula (4) or (42) is selected from the group consisting of the compounds represented by the following formulae (461) to (467).

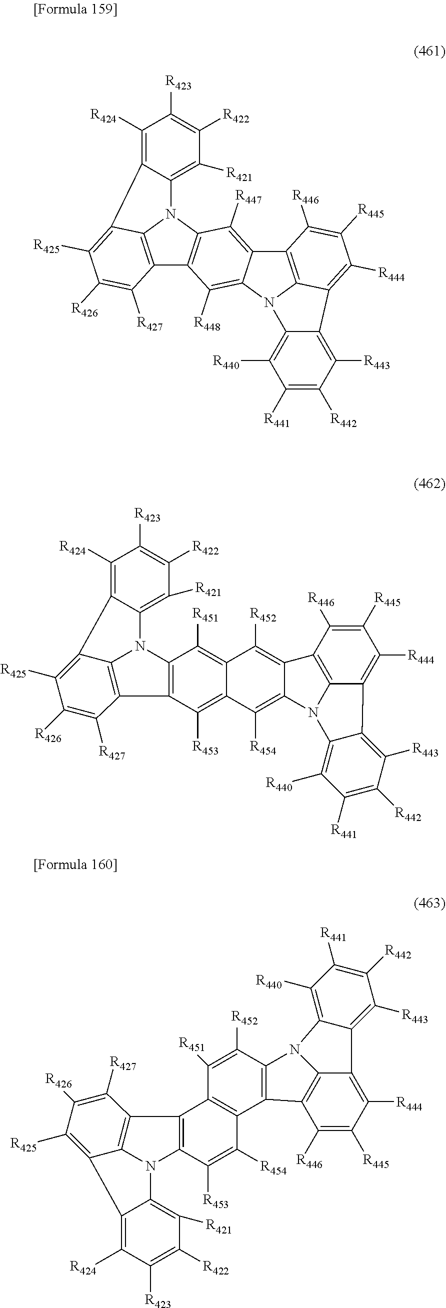

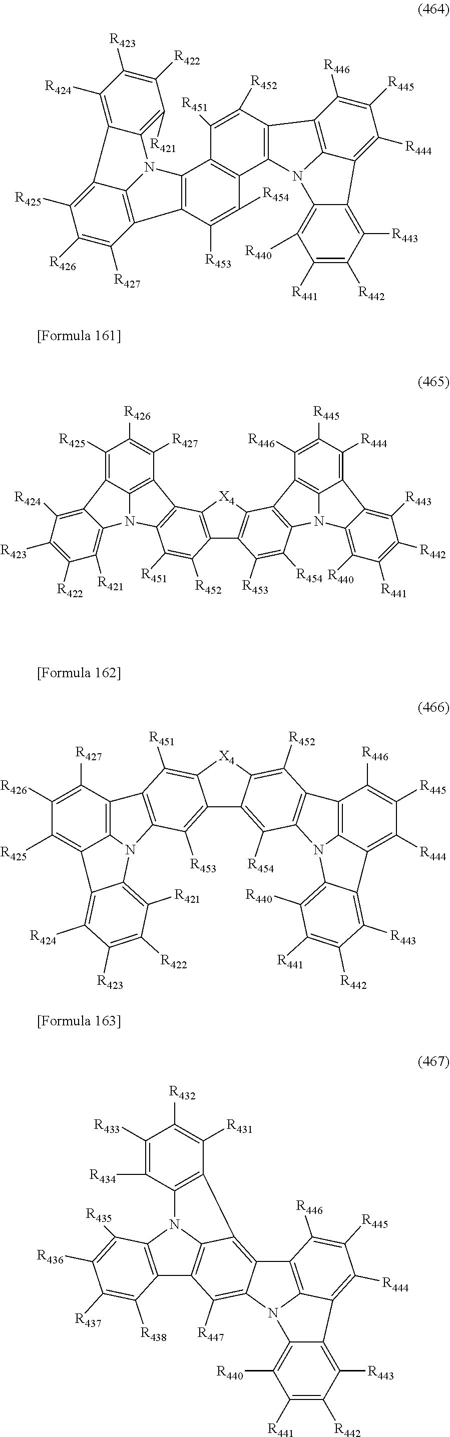

In the formula (461), the formula (462), the formula (463), the formula (464), the formula (465), the formula (466), and the formula (467),
R₄₂₁ to R₄₂₇ represent the same as R₄₂₁ to R₄₂₇, respectively, in the formula (4-1),
R₄₃₁ to R₄₃₈ represent the same as R₄₃₁ to R₄₃₈, respectively, in the formula (4-2),
R₄₄₀ to R₄₄₈ and R₄₅₁ to R₄₅₄ each independently represent the same as R₄₀₁ to R₄₁₁ in the formula (42),
X₄ represents an oxygen atom, NR₈₀₁, or C(R₈₀₂)(R₈₀₃),
R₈₀₁, R₈₀₂ and R₈₀₃ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted aryl group having 6 to 50 ring carbon atoms,
when a plurality of R₈₀₁ are present, the plurality of R₈₀₁ are mutually the same or different,
when a plurality of R₈₀₂ are present, the plurality of R₈₀₂ are mutually the same or different, and
when a plurality of R₈₀₃ are present, the plurality of R₈₀₃ are mutually the same or different.

In an exemplary embodiment, at least one combination of adjacent two or more of R₄₀₁ to R₄₁₁ in a compound represented by the formula (42) are mutually bonded to form a substituted or unsubstituted monocyclic ring, or are mutually bonded to form a substituted or unsubstituted fused ring. This exemplary embodiment is described in detail below with respect to a compound represented by the following formula (45).

Compound Represented by Formula (45)

A compound represented by the formula (45) is described below.

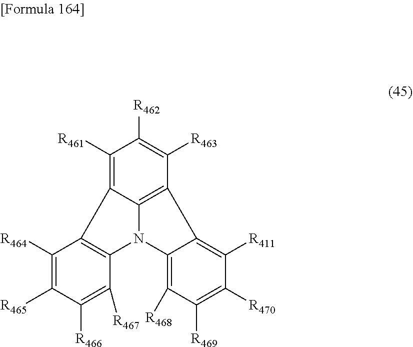

In the formula (45),
two or more combinations selected from the group consisting of a combination of R₄₆₁ and R₄₆₂, a combination of R₄₆₂ and R₄₆₃, a combination of R₄₆₄ and R₄₆₅, a combination of R₄₆₅ and R₄₆₆, a combination of R₄₆₆ and R₄₆₇, a combination of R₄₆₈ and R₄₆₉, a combination of R₄₆₉ and R₄₇₀, and a combination of R₄₇₀ and R₄₇₁ are mutually bonded to form a substituted or unsubstituted monocyclic ring or a substituted or unsubstituted fused ring,
a combination of R₄₆₁ and R₄₆₂ and a combination of R₄₆₂ and R₄₆₃, a combination of R₄₆₄ and R₄₆₅ and a combination of R₄₆₅ and R₄₆₆, a combination of R₄₆₅ and R₄₆₆ and a combination of R₄₆₆ and R₄₆₇, a combination of R₄₆₈ and R₄₆₉ and a combination of R₄₆₉ and R₄₇₀, and a combination of R₄₆₉ and R₄₇₀ and a combination of R₄₇₀ and R₄₇₁ do not simultaneously form a ring,
two or more rings formed of R₄₆₁ to R₄₇₁ are mutually the same or different, and
R₄₆₁ to R₄₇₁ that do not form the monocyclic ring and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅) or —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (45), Rₙ and Rₙ₊₁ (n represents an integer selected from 461, 462, 464 to 466, and 468 to 470) are mutually bonded and, together with two ring carbon atoms bonded to Rₙ and Rₙ₊₁ form a substituted or unsubstituted monocyclic ring or a substituted or unsubstituted fused ring. The ring is preferably composed of atoms selected from the group consisting of a carbon atom, an oxygen atom, a sulfur atom, and a nitrogen atom, and the number of atoms in the ring preferably ranges from 3 to 7, more preferably 5 or 6.

The number of the ring structures in a compound represented by the formula (45) is two, three, or four, for example. The two or more ring structures may be present on the same benzene ring or different benzene rings on the parent skeleton of the formula (45). For example, for three ring structures, one ring structure may be present in each of the three benzene rings of the formula (45).

The ring structures in a compound represented by the formula (45) are structures represented by the following formulae (451) to (460), for example.

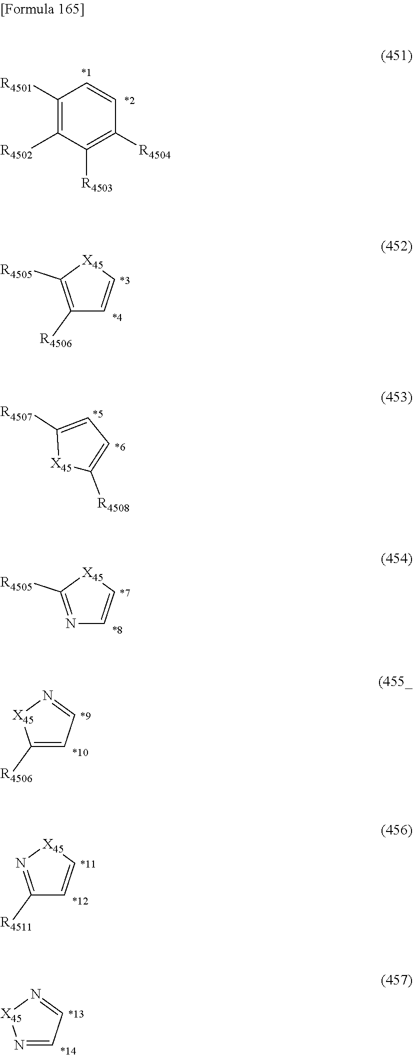

In the formulae (451) to (457),
*1 and *2, *3 and *4, *5 and *6, *7 and *8, *9 and *10, *11 and *12, and *13 and *14 independently represent the two ring carbon atoms bonded to Rₙ and Rₙ₊₁,
the ring carbon atom bonded to Rn may be one of the two ring carbon atoms represented by *1 and *2, *3 and *4, *5 and *6, *7 and *8, *9 and *10, *11 and *12, and *13 and *14,
X₄₅ represents C(R₄₅₁₂)(R₄₅₁₃), NR₄₅₁₄, an oxygen atom, or a sulfur atom,
at least one combination of adjacent two or more of R₄₅₀₁ to R₄₅₀₆ and R₄₅₁₂ to R₄₅₁₃ are mutually bonded to form a substituted or unsubstituted monocyclic ring, are mutually bonded to form a substituted or unsubstituted fused ring, or are not mutually bonded, and
R₄₅₀₁ to R₄₅₁₄ that do not form the monocyclic ring and do not form the fused ring each independently represent the same as R₄₆₁ to R₄₇₁ in the formula (45).

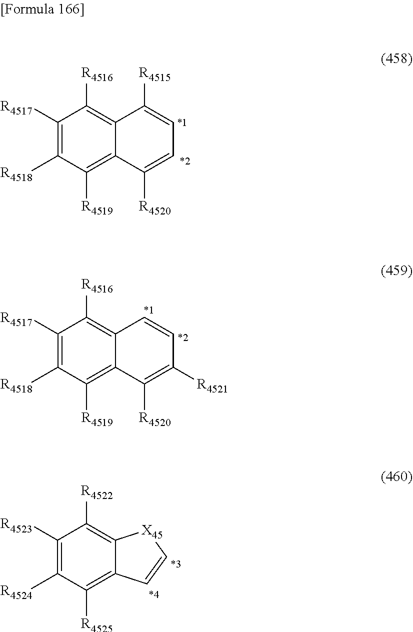

In the formulae (458) to (460),
*1 and *2, and *3 and *4 independently represent the two ring carbon atoms bonded to Rₙ and Rₙ₊₁,
the ring carbon atom bonded to Rn may be one of the two ring carbon atoms represented by *1 and *2, or *3 and *4,
X₄₅ represents C(R₄₅₁₂)(R₄₅₁₃), NR₄₅₁₄, an oxygen atom, or a sulfur atom,
at least one combination of adjacent two or more of R₄₅₁₂ to R₄₅₁₃ and R₄₅₁₅ to R₄₅₂₅ are mutually bonded to form a substituted or unsubstituted monocyclic ring, are mutually bonded to form a substituted or unsubstituted fused ring, or are not mutually bonded, and
R₄₅₁₂ to R₄₅₁₃, R₄₅₁₅ to R₄₅₂₁, R₄₅₂₂ to R₄₅₂₅, and R₄₅₁₄ that do not form the monocyclic ring and do not form the fused ring each independently represent the same as R₄₆₁ to R₄₇₁ in the formula (45).

In the formula (45), at least one of R₄₆₂, R₄₆₄, R₄₆₅, R₄₇₀, or R₄₇₁ (preferably at least one of R₄₆₂, R₄₆₅, or R₄₇₀, more preferably R₄₆₂) is preferably a group that does not form a ring structure.

It is preferable that (i) a substituent of a ring structure formed by Rₙ and Rₙ₊₁ having the substituent in the formula (45), (ii) R₄₆₁ to R₄₇₁ that do not form a ring structure in the formula (45), and (iii) R₄₅₀₁ to R₄₅₁₄ and R₄₅₁₅ to R₄₅₂₅ in the formulae (451) to (460) each independently represent a group selected from the group consisting of a hydrogen atom, substituted or unsubstituted alkyl group having 1 to 50 carbon atoms, substituted or unsubstituted alkenyl group having 2 to 50 carbon atoms, substituted or unsubstituted alkynyl group having 2 to 50 carbon atoms, substituted or unsubstituted cycloalkyl group having 3 to 50 ring carbon atoms, group represented by —N(R₉₀₆)(R₉₀₇), substituted or unsubstituted aryl group having 6 to 50 ring carbon atoms, substituted or unsubstituted heterocyclic group having 5 to 50 ring atoms, and group represented by the following formulae (461) to (464).

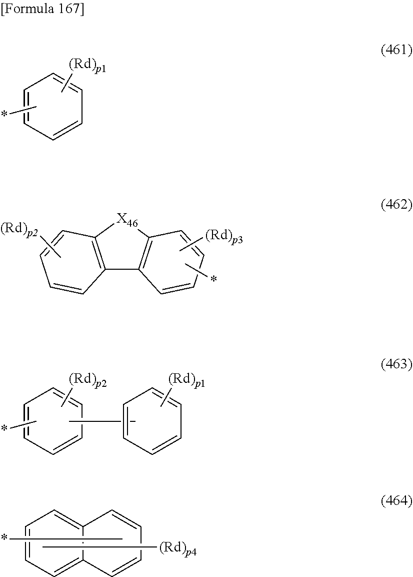

In the formulae (461) to (464),
R_{d} each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms,
X₄₆ represents C(R₈₀₁)(R₈₀₂), NR₈₀₃, an oxygen atom, or a sulfur atom,
R₈₀₁, R₈₀₂ and R₈₀₃ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted aryl group having 6 to 50 ring carbon atoms,
when a plurality of R₈₀₁ are present, the plurality of R₈₀₁ are mutually the same or different,
when a plurality of R₈₀₂ are present, the plurality of R₈₀₂ are mutually the same or different,
when a plurality of R₈₀₃ are present, the plurality of R₈₀₃ are mutually the same or different,
p1 is 5, p2 is 4, p3 is 3, p4 is 7, and
* in the formulae (461) to (464) each independently represents a position of bonding to a ring structure.

In the fluorescent compound (the compound M1), R₉₀₁ to R₉₀₇ are as defined above.

In an exemplary embodiment, a compound represented by the formula (45) is represented by one of the following formulae (45-1) to (45-6).

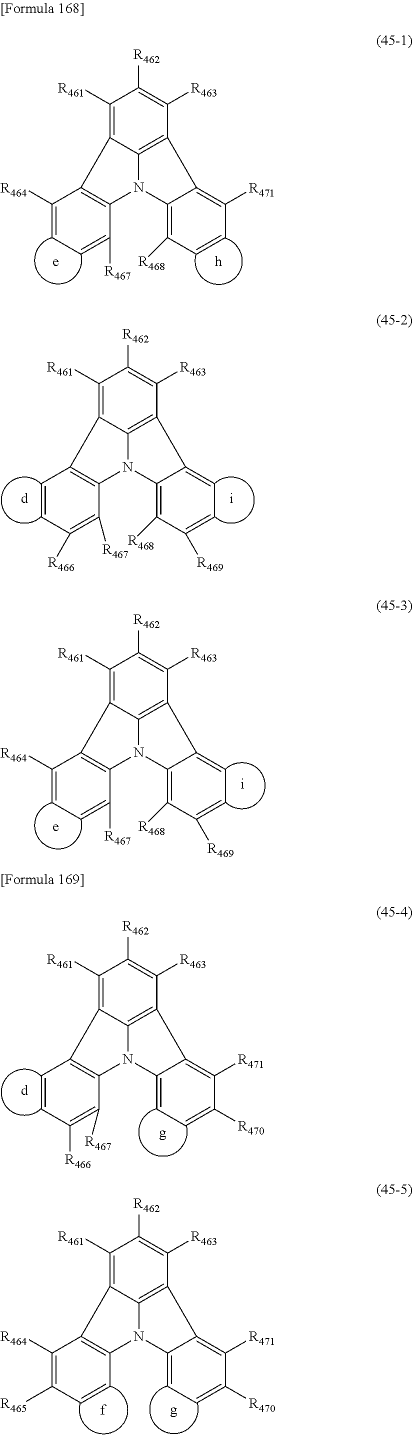

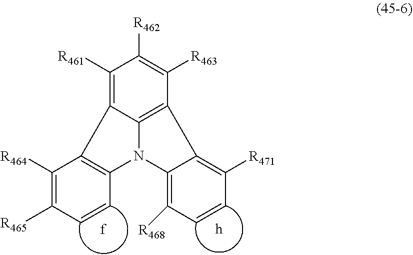

In the formulae (45-1) to (45-6), rings d to i each independently represent a substituted or unsubstituted monocyclic ring or a substituted or unsubstituted fused ring, and R₄₆₁ to R₄₇₁ represent the same as R₄₆₁ to R₄₇₁, respectively, in the formula (45).

In an exemplary embodiment, a compound represented by the formula (45) is represented by one of the following formulae (45-7) to (45-12).

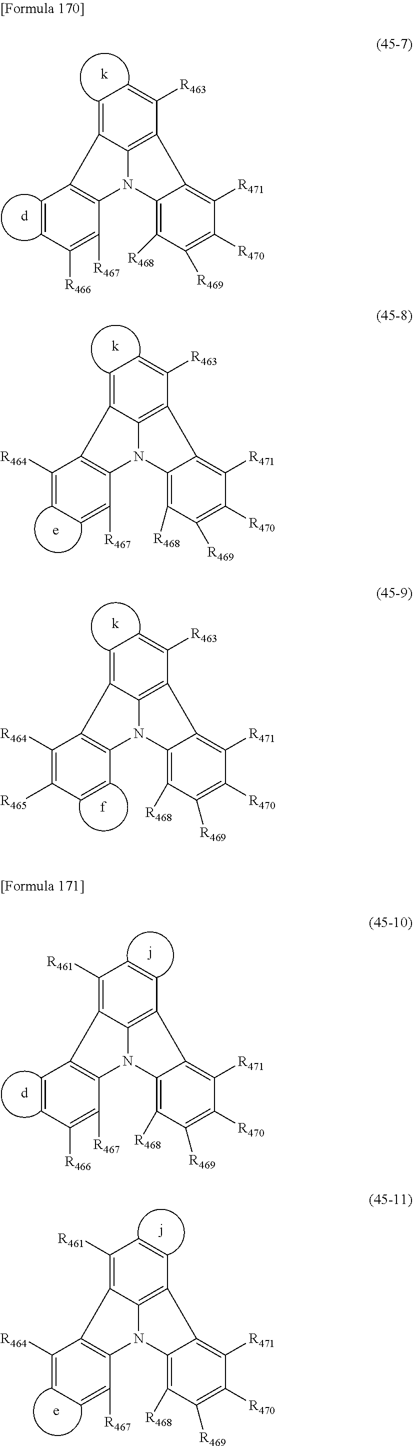

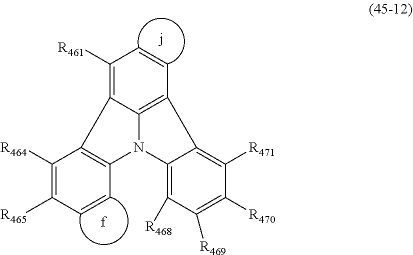

In the formulae (45-7) to (45-12), rings d to f, k, and j each independently represent a substituted or unsubstituted monocyclic ring or a substituted or unsubstituted fused ring, and R₄₆₁ to R₄₇₁ represent the same as R₄₆₁ to R₄₇₁, respectively, in the formula (45).

In an exemplary embodiment, a compound represented by the formula (45) is represented by one of the following formulae (45-13) to (45-21).

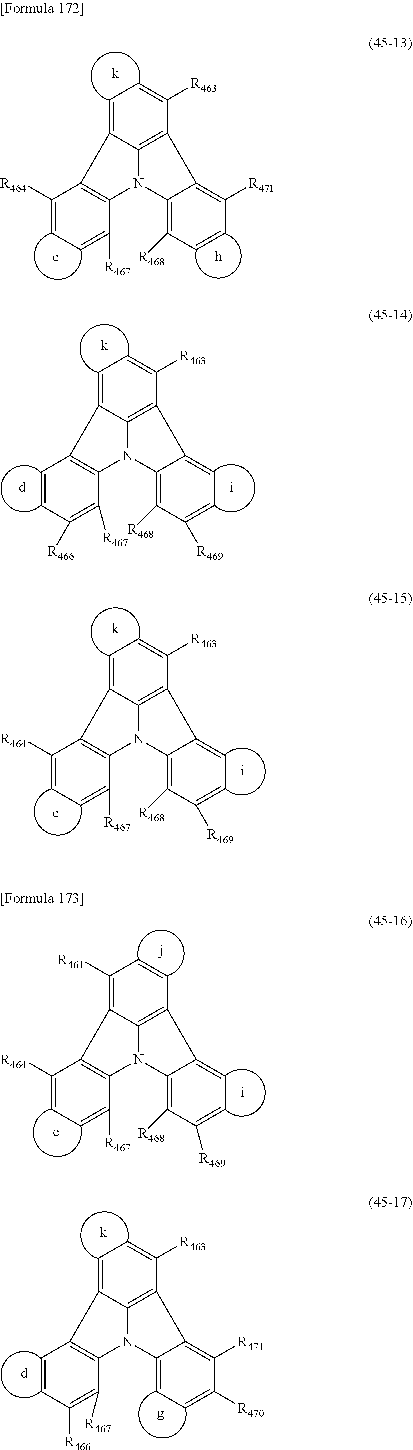

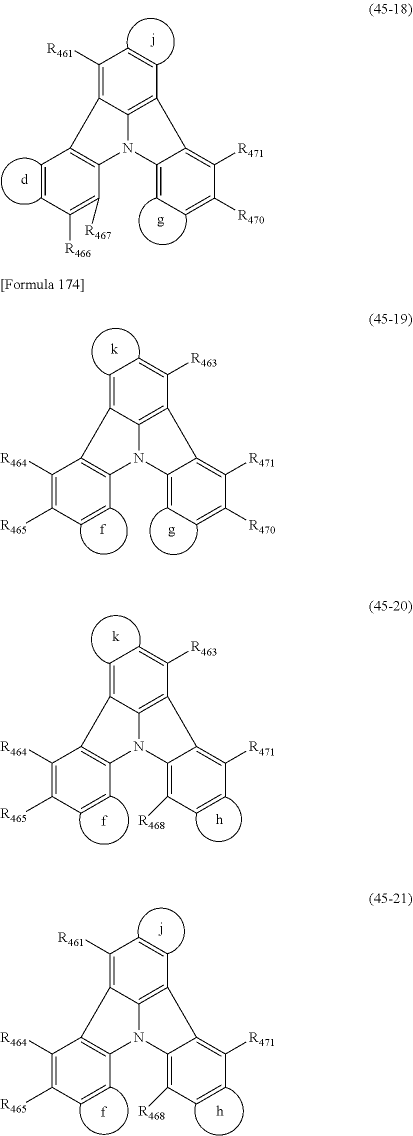

In the formulae (45-13) to (45-21), rings d to k each independently represent a substituted or unsubstituted monocyclic ring or a substituted or unsubstituted fused ring, and R₄₆₁ to R₄₇₁ represent the same as R₄₆₁ to R₄₇₁, respectively, in the formula (45).

When the ring g or the ring h further has a substituent, the substituent is, for example, a substituted or unsubstituted alkyl group having 1 to 50 carbon atoms, a substituted or unsubstituted aryl group having 6 to 50 ring carbon atoms, a group represented by the formula (461), a group represented by the formula (463), or a group represented by the formula (464).

In an exemplary embodiment, a compound represented by the formula (45) is represented by one of the following formulae (45-22) to (45-25).

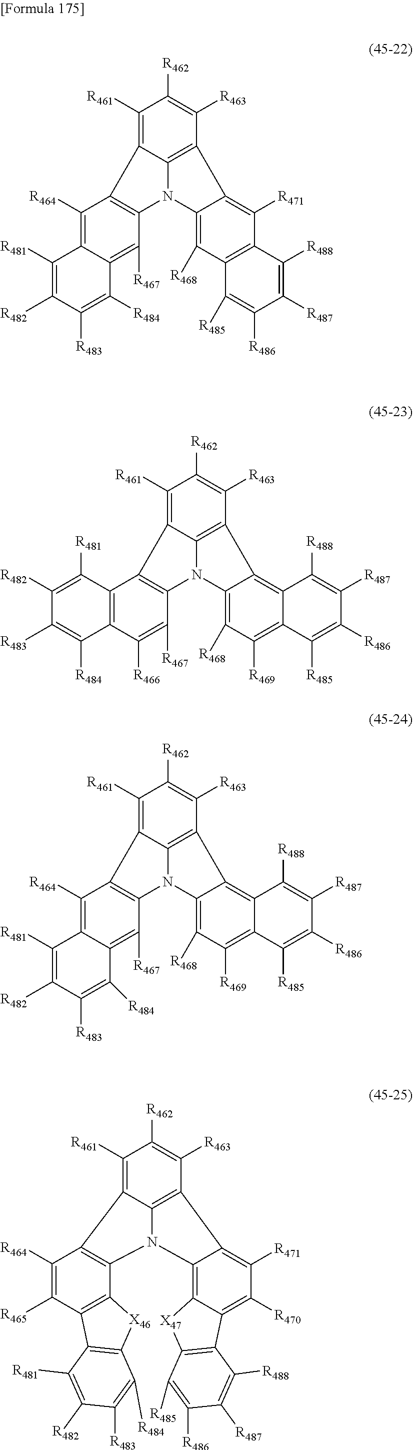

In the formulae (45-22) to (45-25),
X₄₆ and X₄₇ each independently represent C(R₈₀₁)(R₈₀₂), NR₈₀₃, an oxygen atom, or a sulfur atom,
R₄₆₁ to R₄₇₁ and R₄₈₁ to R₄₈₈ each independently represent the same as R₄₆₁ to R₄₇₁ in the formula (45),
R₈₀₁, R₈₀₂ and R₈₀₃ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted aryl group having 6 to 50 ring carbon atoms,
when a plurality of R₈₀₁ are present, the plurality of R₈₀₁ are mutually the same or different,
when a plurality of R₈₀₂ are present, the plurality of R₈₀₂ are mutually the same or different, and
when a plurality of R₈₀₃ are present, the plurality of R₈₀₃ are mutually the same or different.

In an exemplary embodiment, a compound represented by the formula (45) is represented by the following formula (45-26).

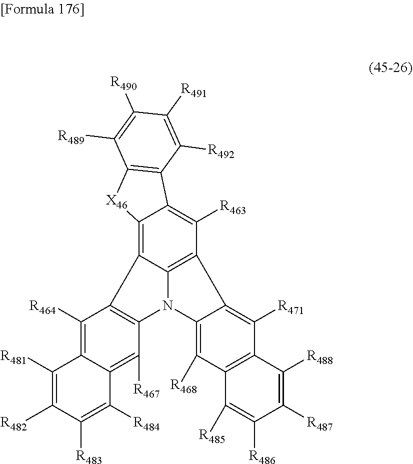

In the formula (45-26),
X₄₆ represents C(R₈₀₁)(R₈₀₂), NR₈₀₃, an oxygen atom, or a sulfur atom, and
R₄₆₃, R₄₆₄, R₄₆₇, R₄₆₈, R₄₇₁, and R₄₈₁ to R₄₉₂ each independently represent the same as R₄₆₁ to R₄₇₁ in the formula (45).
R₈₀₁, R₈₀₂, and R₈₀₃ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted aryl group having 6 to 50 ring carbon atoms,
when a plurality of R₈₀₁ are present, the plurality of R₈₀₁ are mutually the same or different,
when a plurality of R₈₀₂ are present, the plurality of R₈₀₂ are mutually the same or different, and
when a plurality of R₈₀₃ are present, the plurality of R₈₀₃ are mutually the same or different.

Specific examples of a compound represented by the formula (4) include the following compounds.

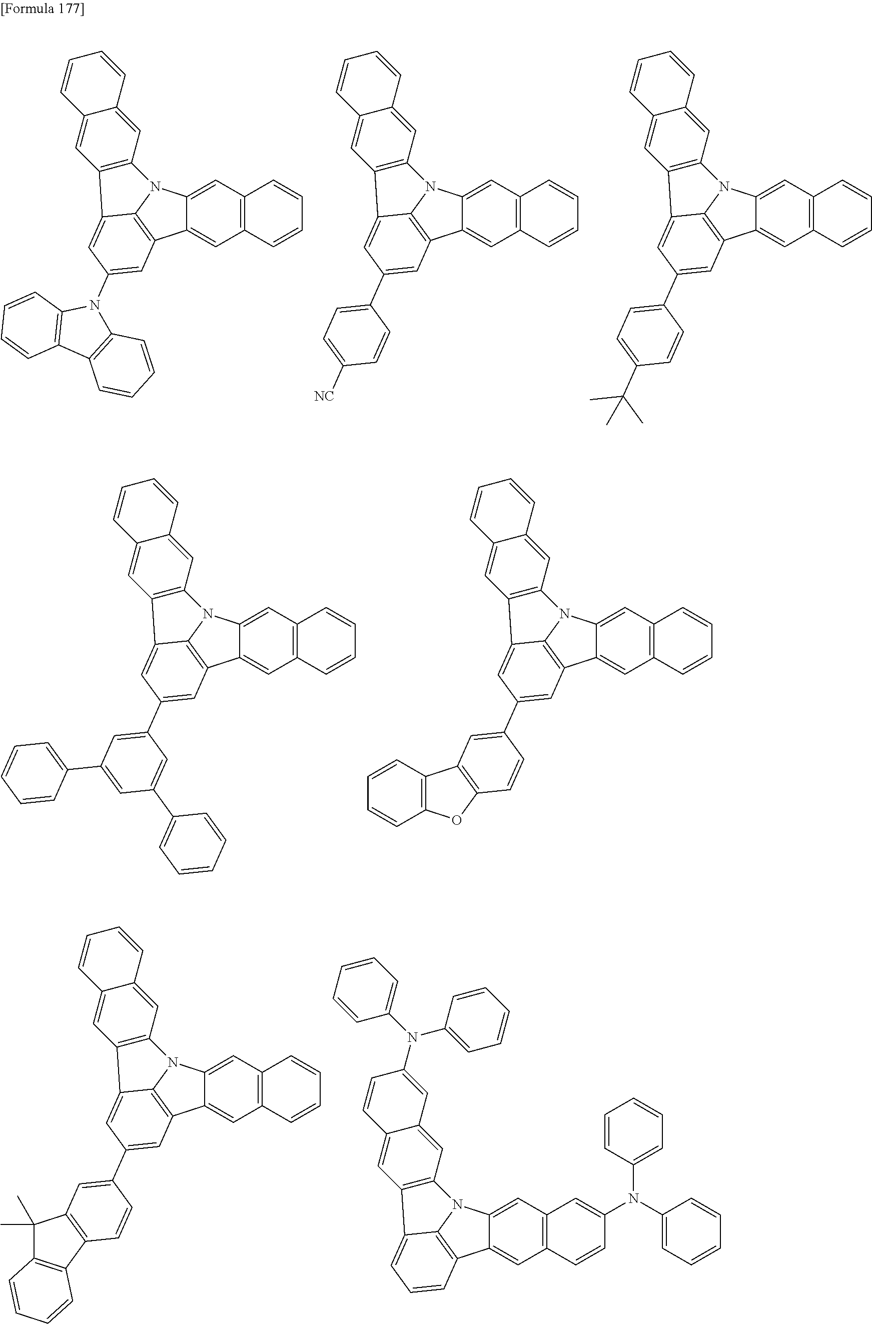

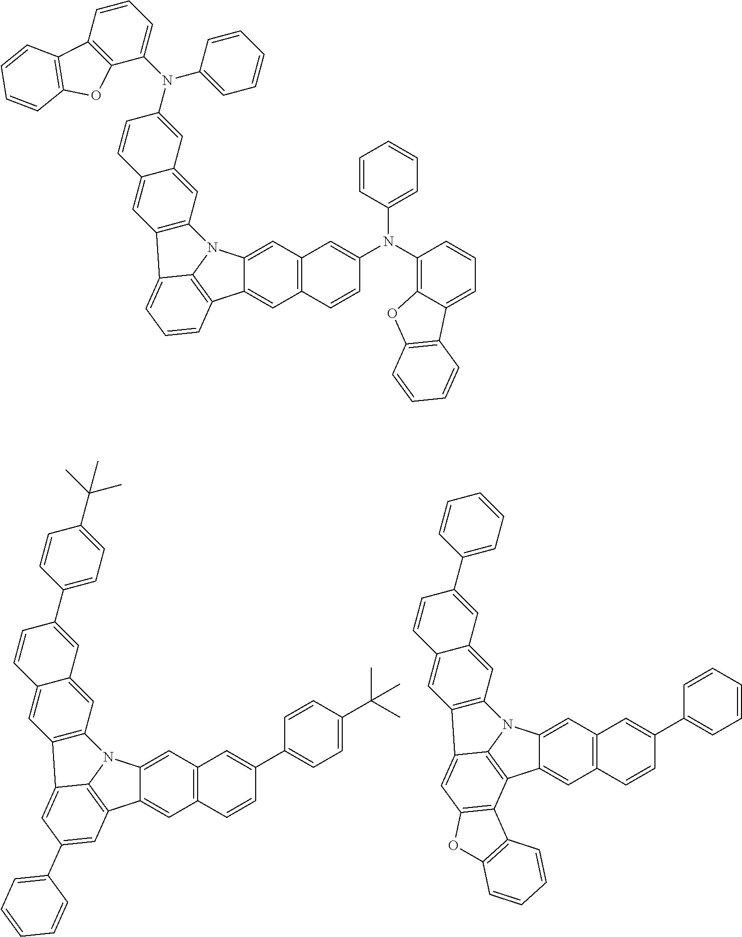

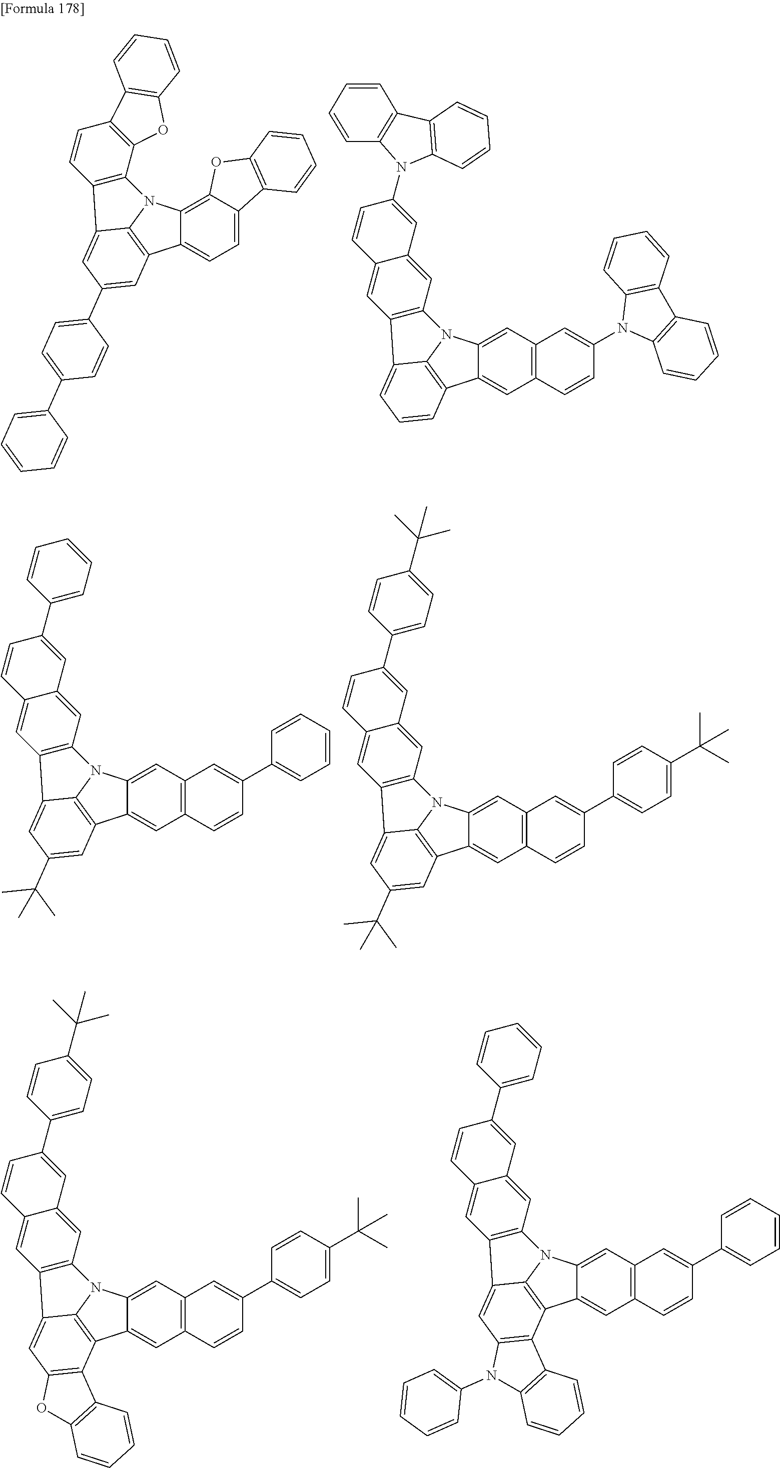

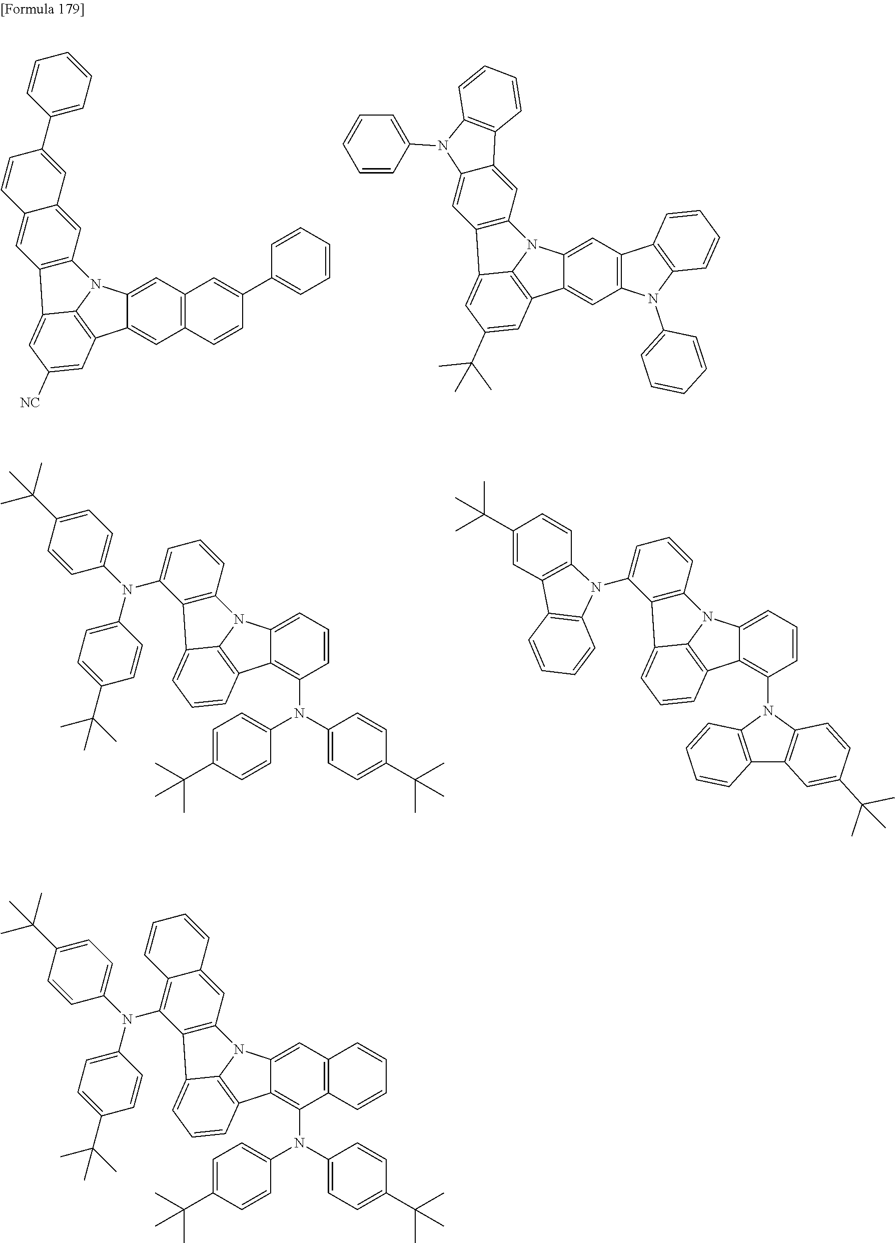

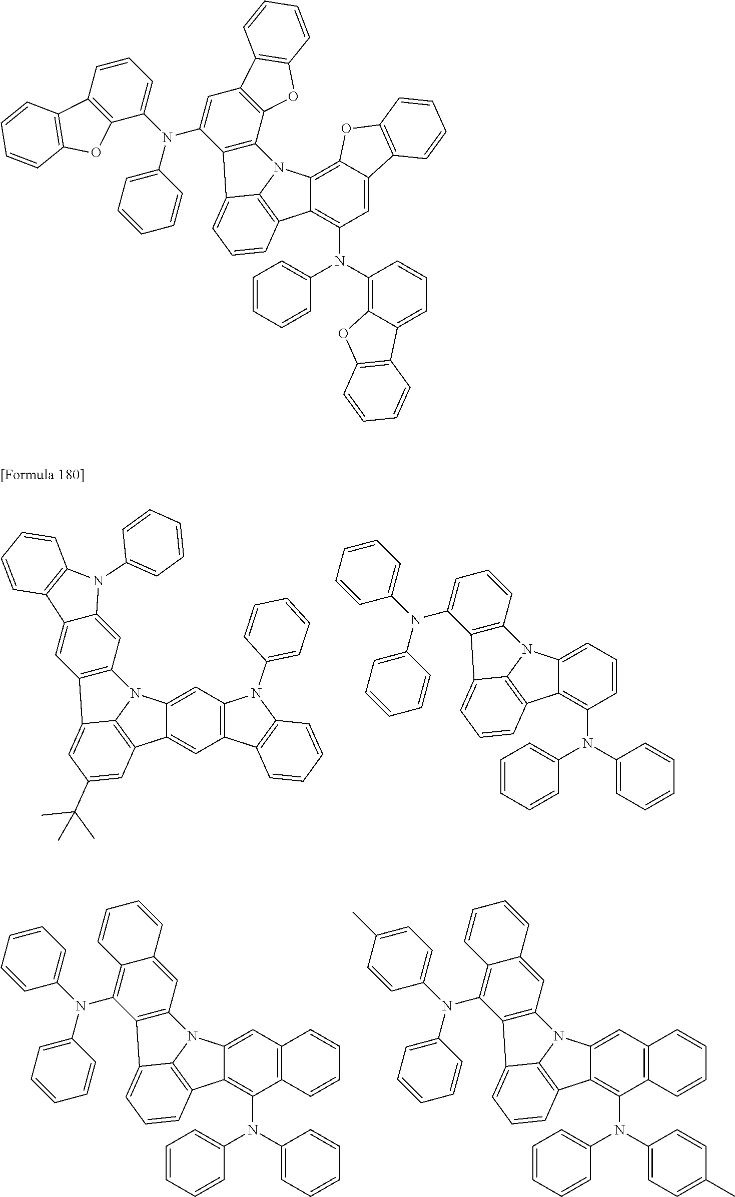

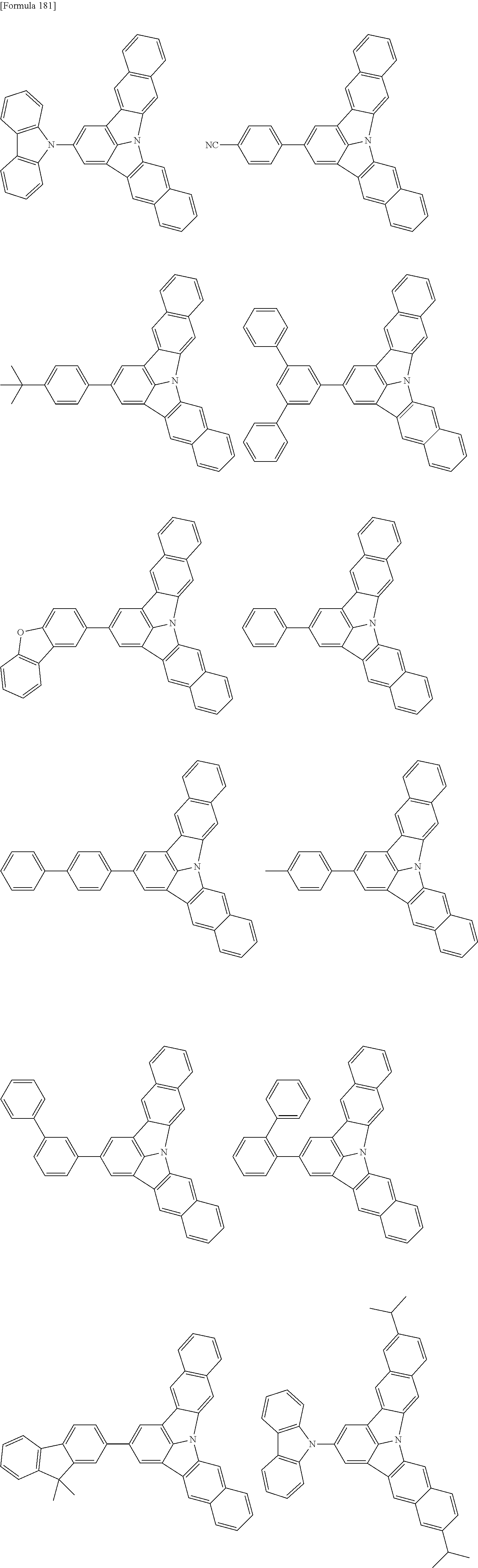

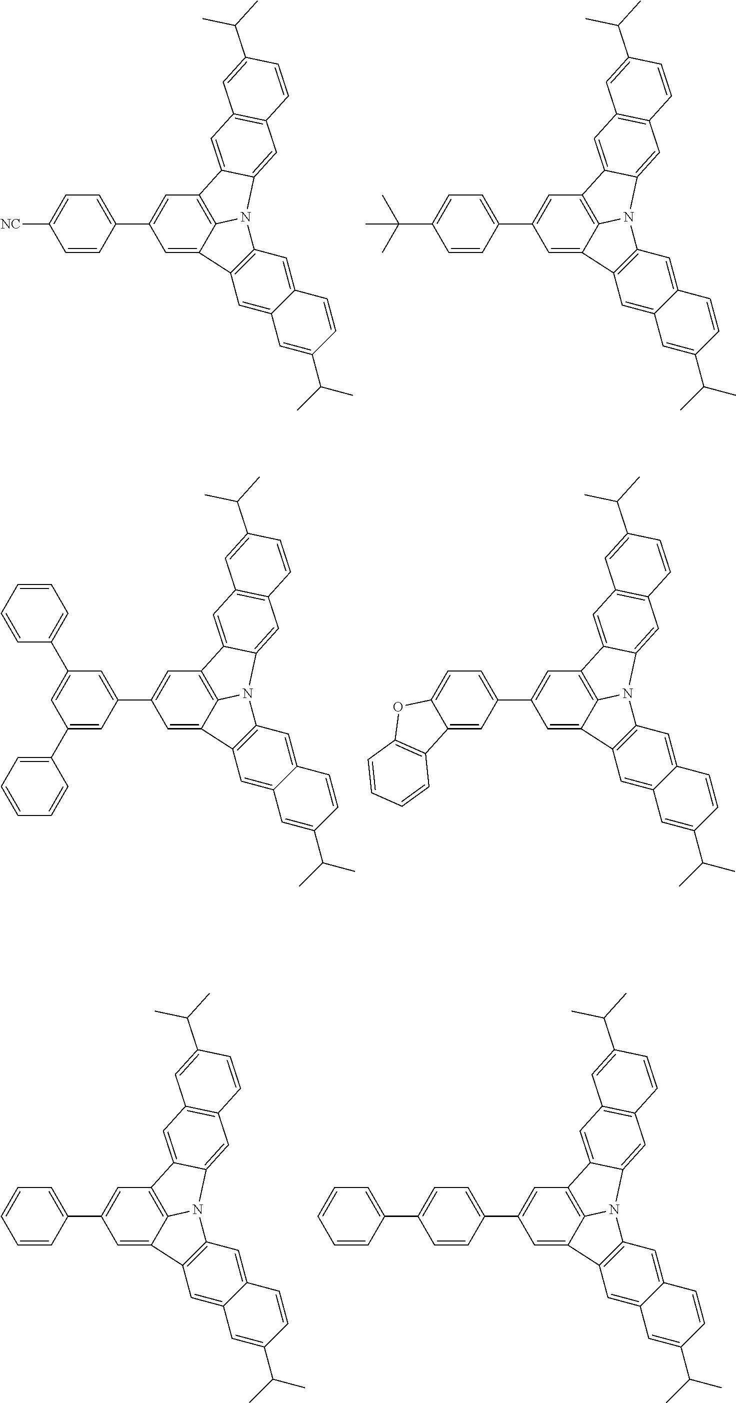

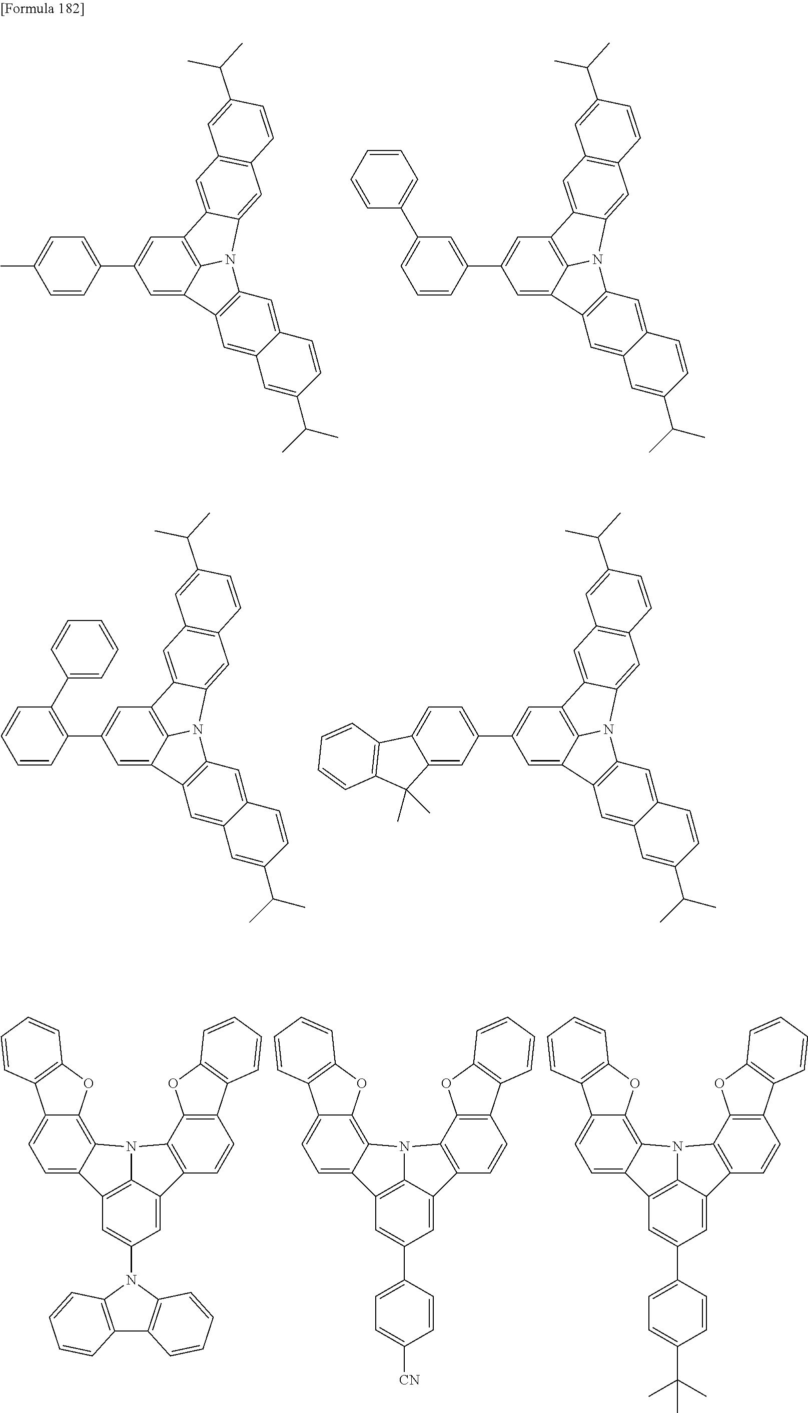

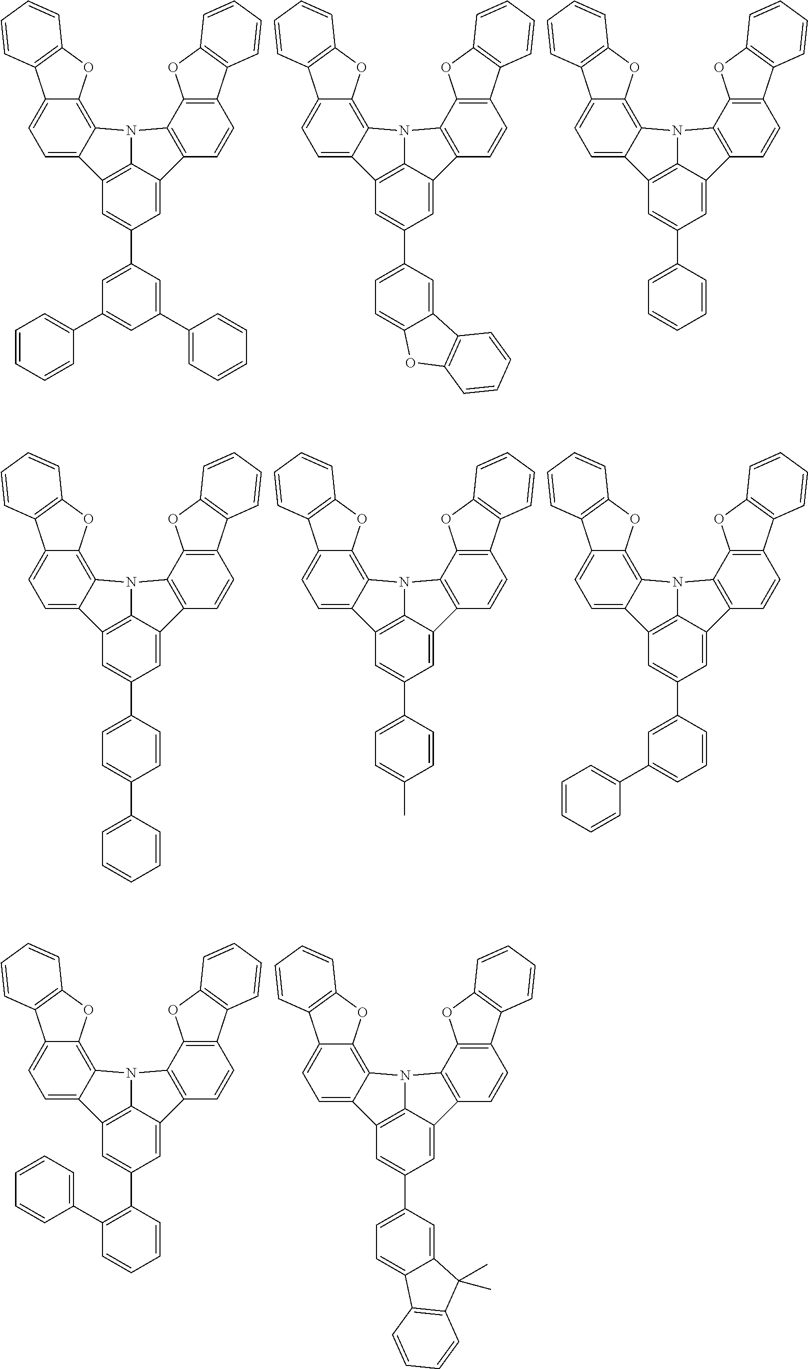

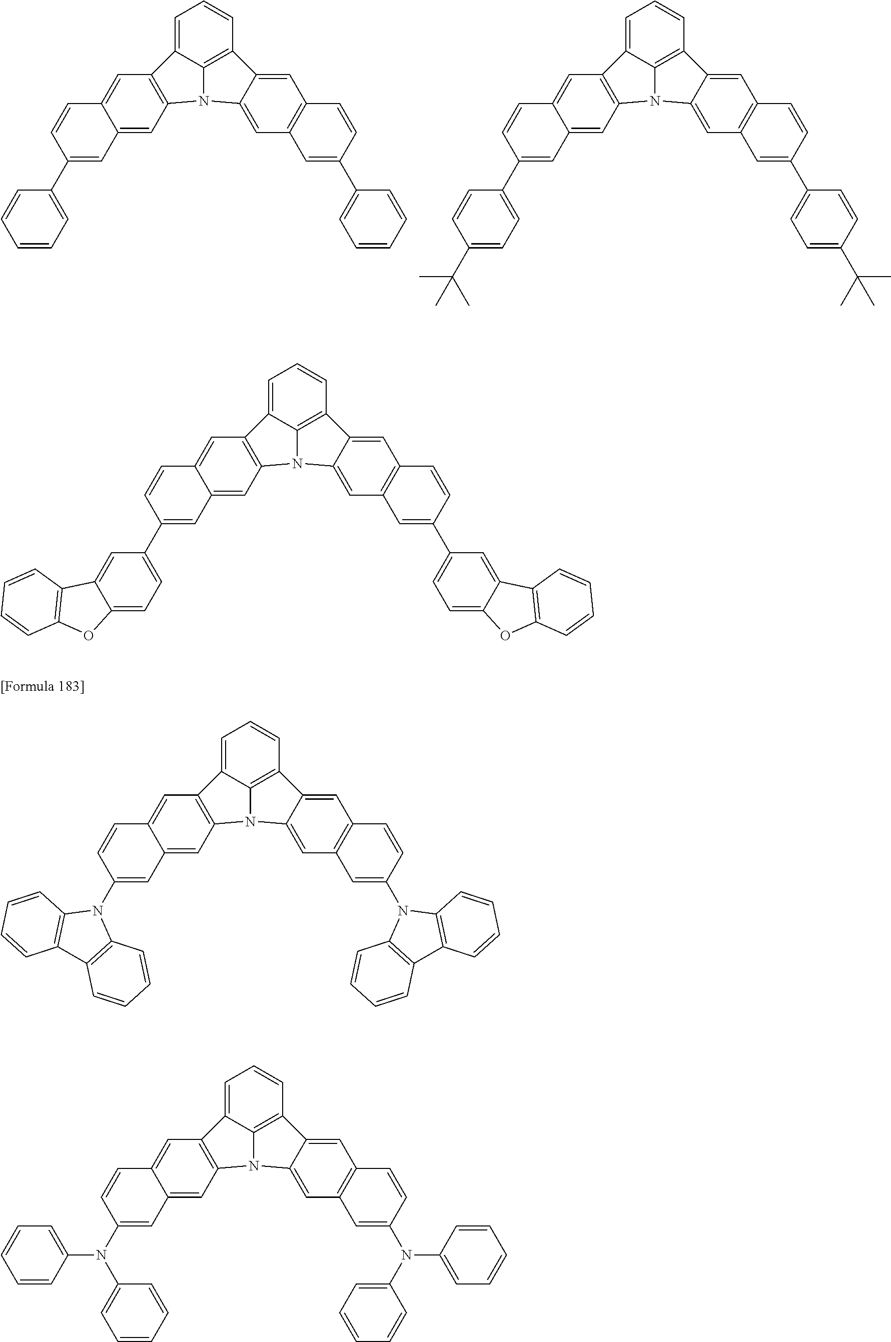

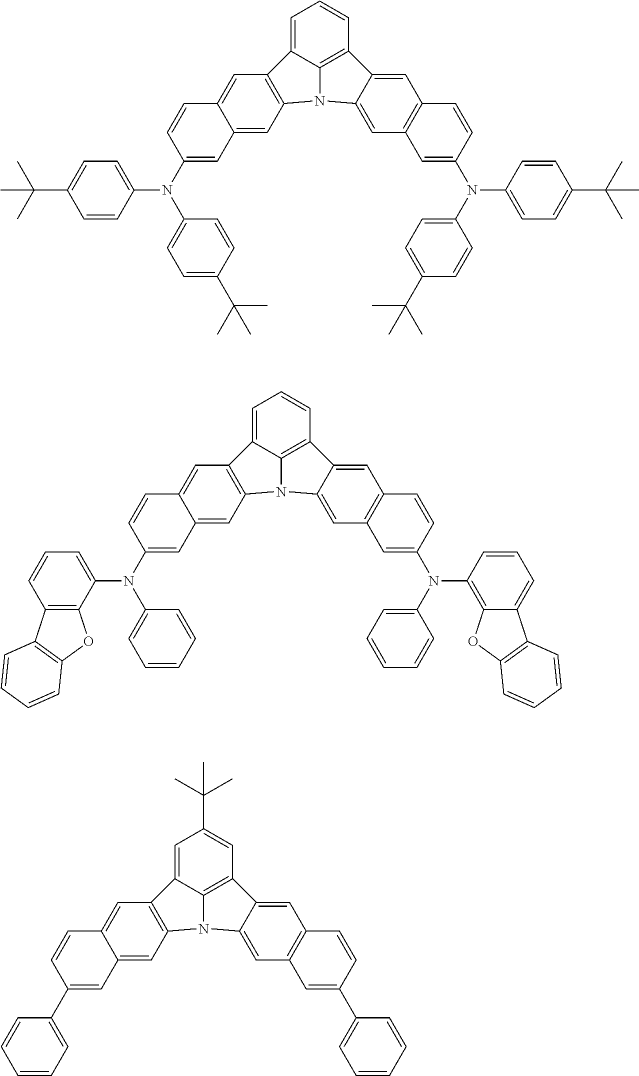 [SMILES: CC(C)(C)c1ccc(N(c2ccc(C(C)(C)C)cc2)c2ccc3cc4c5cccc6c7cc8ccc(N(c9ccc(C(C)(C)C)cc9)c9ccc(C(C)(C)C)cc9)cc8cc7n(c4cc3c2)c56)cc1 ; c1ccc(N(c2ccc3cc4c5cccc6c7cc8ccc(N(c9ccccc9)c9cccc%10c9oc9ccccc9%10)cc8cc7n(c4cc3c2)c56)c2cccc3c2oc2ccccc23)cc1 ; CC(C)(C)c1cc2c3cc4ccc(-c5ccccc5)cc4cc3n3c4cc5cc(-c6ccccc6)ccc5cc4c(c1)c23]

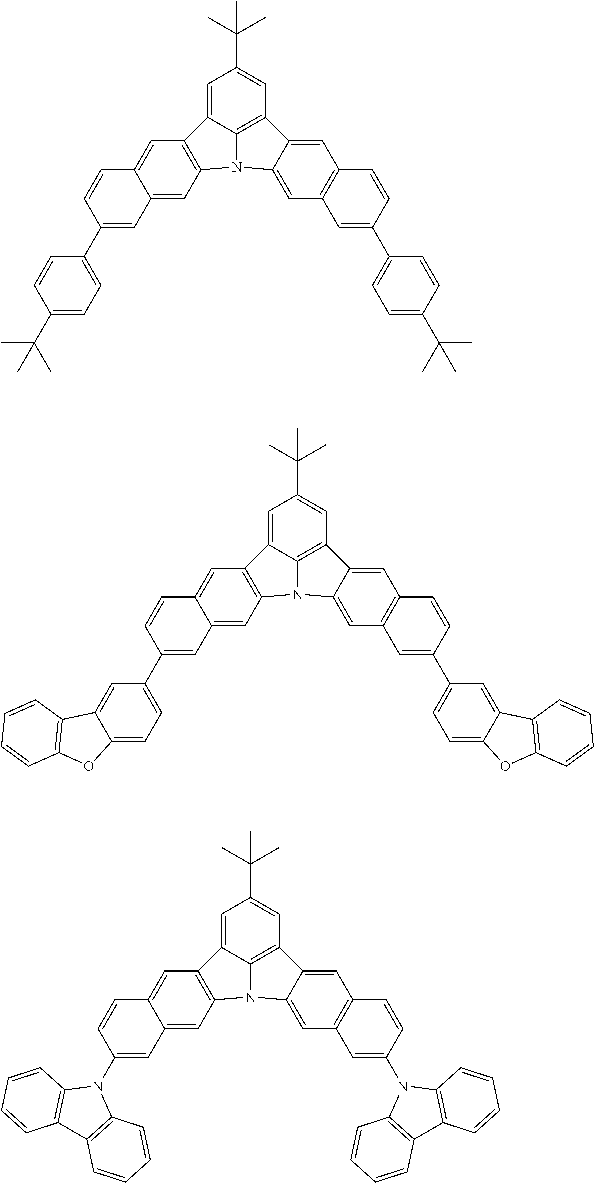 [SMILES: CC(C)(C)c1ccc(-c2ccc3cc4c5cc(C(C)(C)C)cc6c7cc8ccc(-c9ccc(C(C)(C)C)cc9)cc8cc7n(c4cc3c2)c56)cc1 ; CC(C)(C)c1cc2c3cc4ccc(-c5ccc6oc7ccccc7c6c5)cc4cc3n3c4cc5cc(-c6ccc7oc8ccccc8c7c6)ccc5cc4c(c1)c23 ; CC(C)(C)c1cc2c3cc4ccc(-n5c6ccccc6c6ccccc65)cc4cc3n3c4cc5cc(-n6c7ccccc7c7ccccc76)ccc5cc4c(c1)c23]

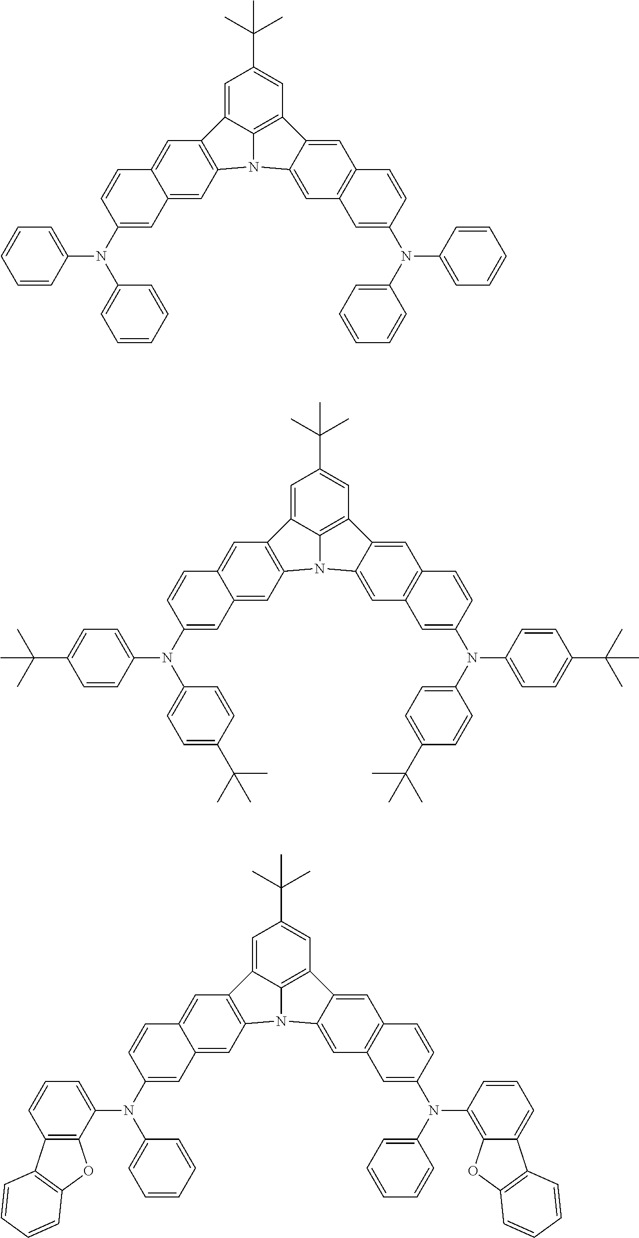 [SMILES: CC(C)(C)c1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C(C)(C)C)cc6c7cc8ccc(N(c9ccc(C(C)(C)C)cc9)c9ccc(C(C)(C)C)cc9)cc8cc7n(c4cc3c2)c56)cc1 ; CC(C)(C)c1cc2c3cc4ccc(N(c5ccccc5)c5cccc6c5oc5ccccc56)cc4cc3n3c4cc5cc(N(c6ccccc6)c6cccc7c6oc6ccccc67)ccc5cc4c(c1)c23]

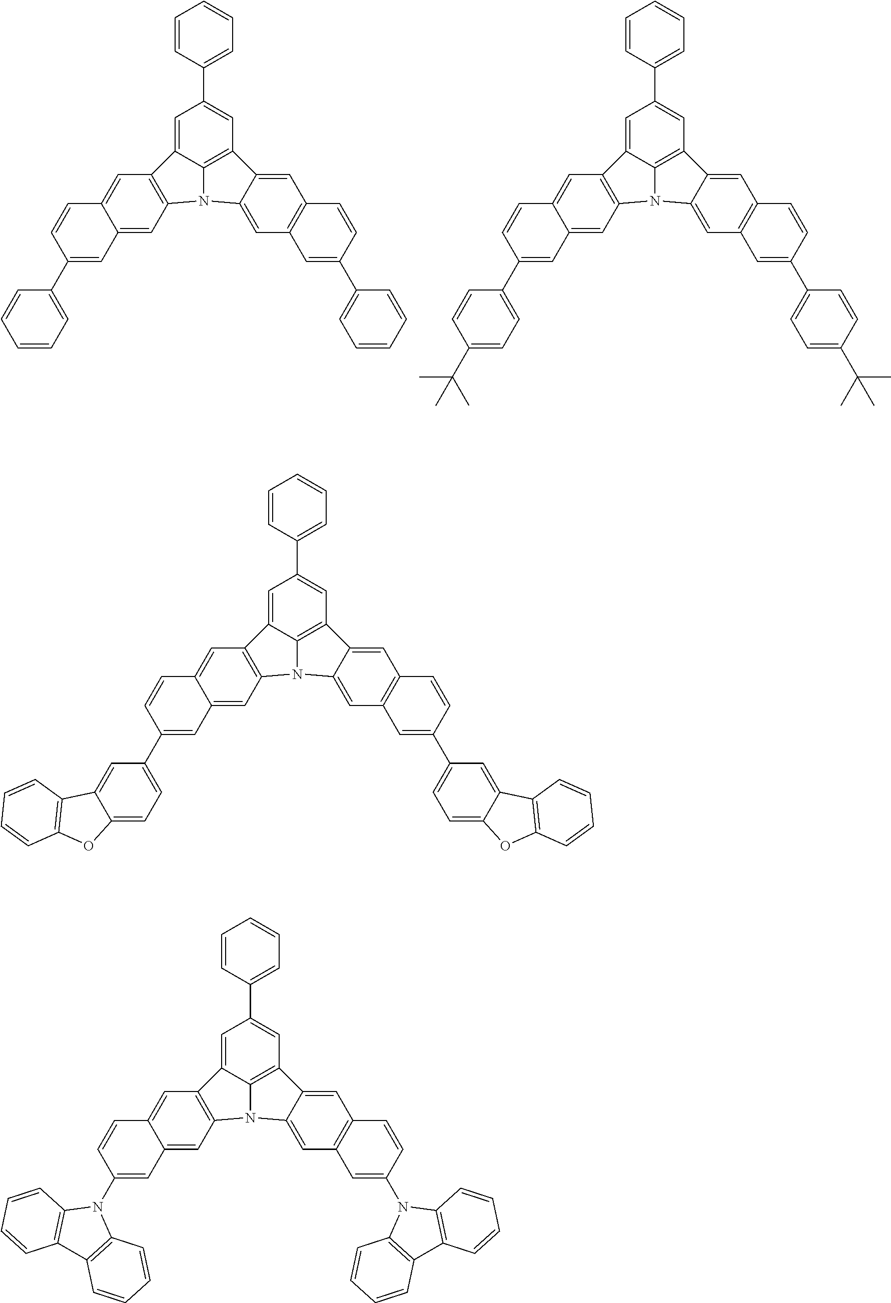

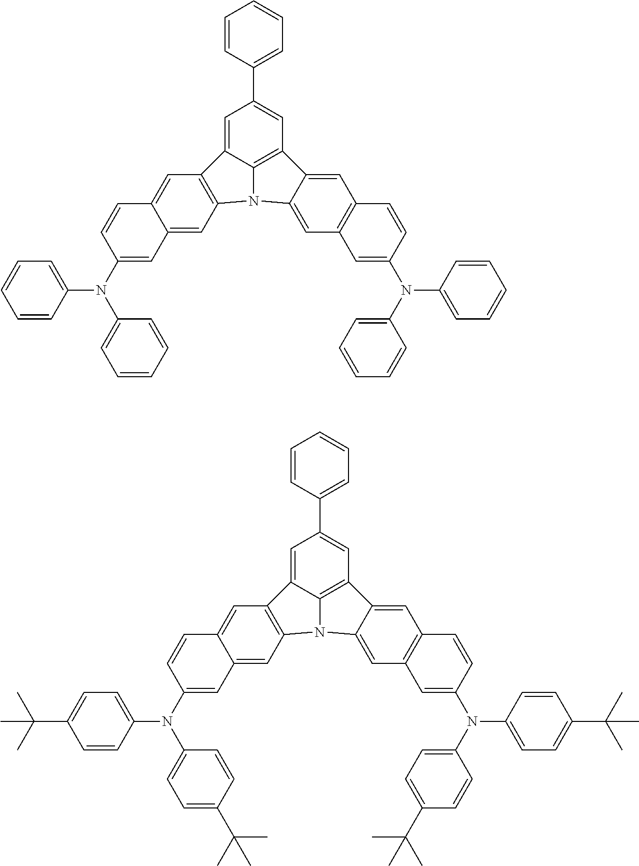 [SMILES: c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; CC(C)(C)c1ccc(N(c2ccc(C(C)(C)C)cc2)c2ccc3cc4c5cc(-c6ccccc6)cc6c7cc8ccc(N(c9ccc(C(C)(C)C)cc9)c9ccc(C(C)(C)C)cc9)cc8cc7n(c4cc3c2)c56)cc1]

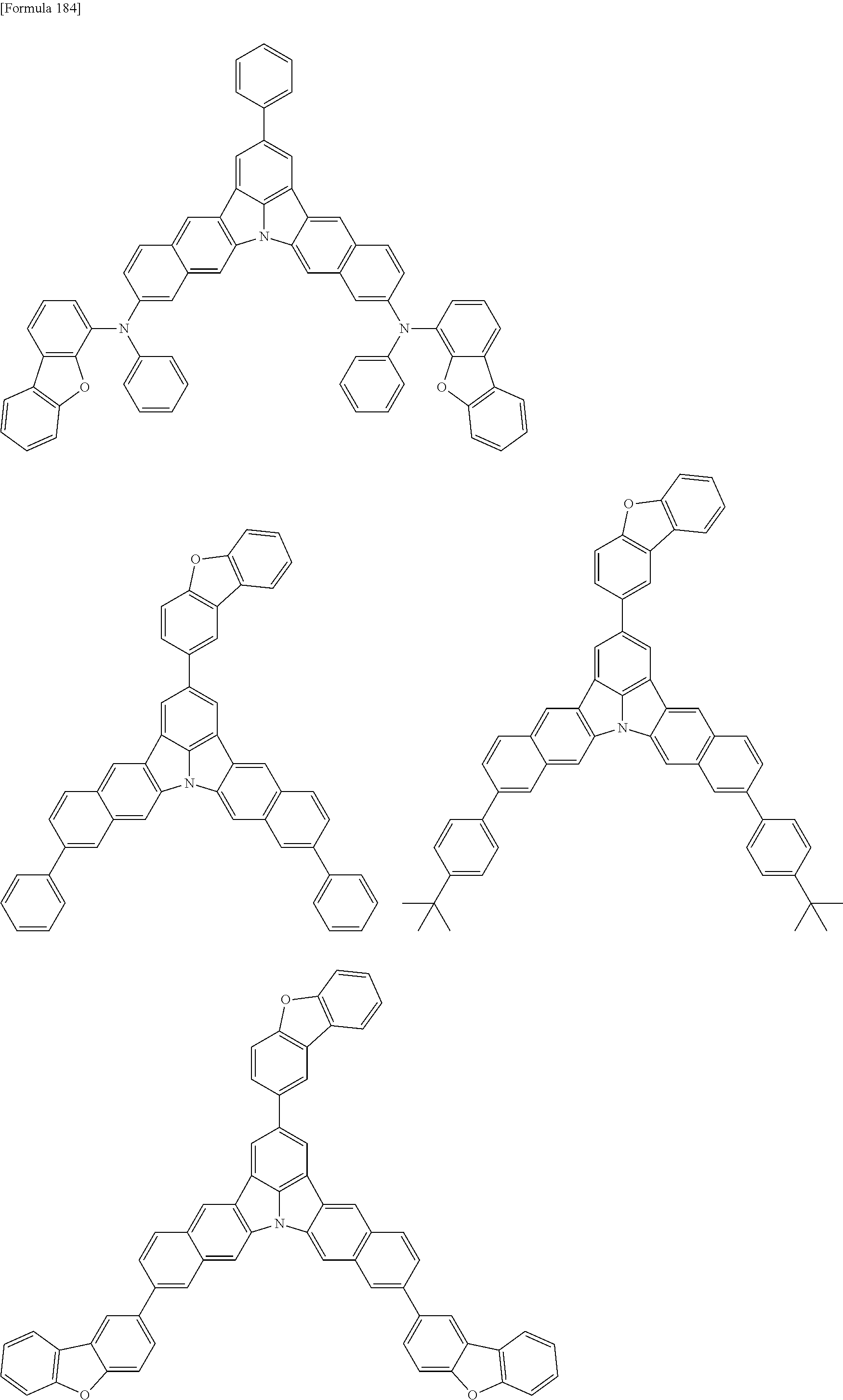

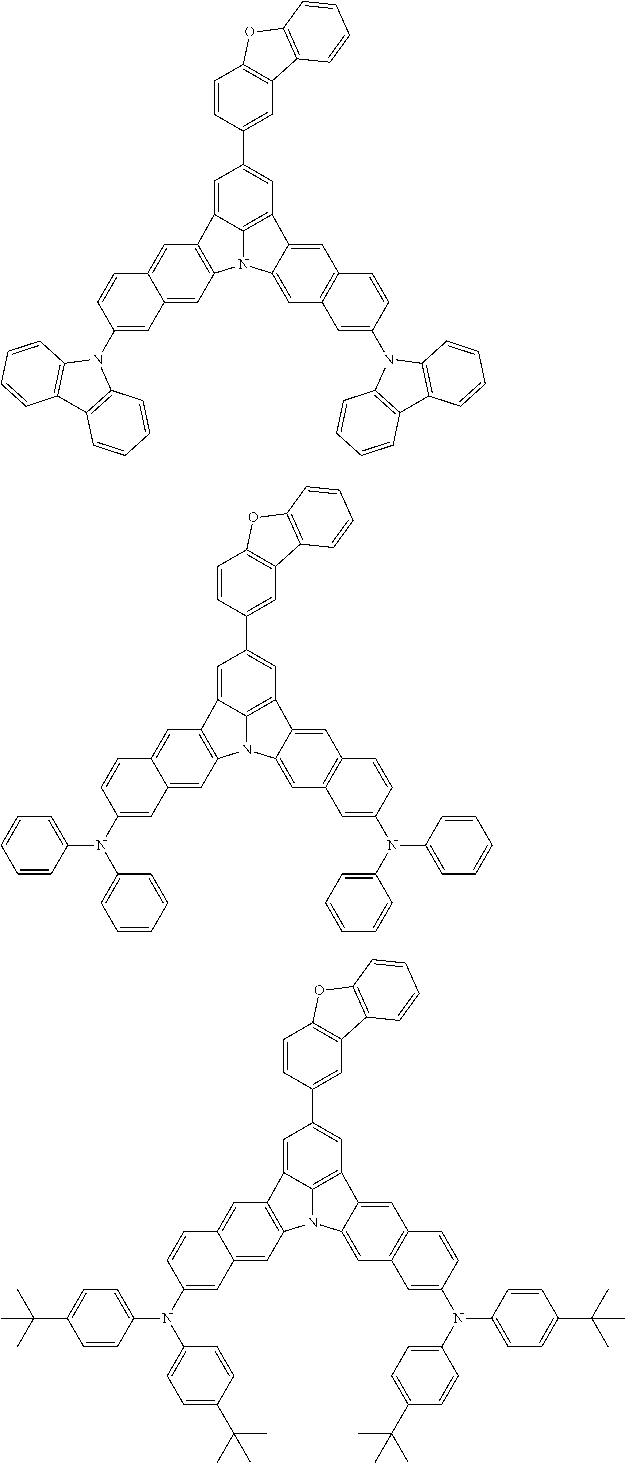

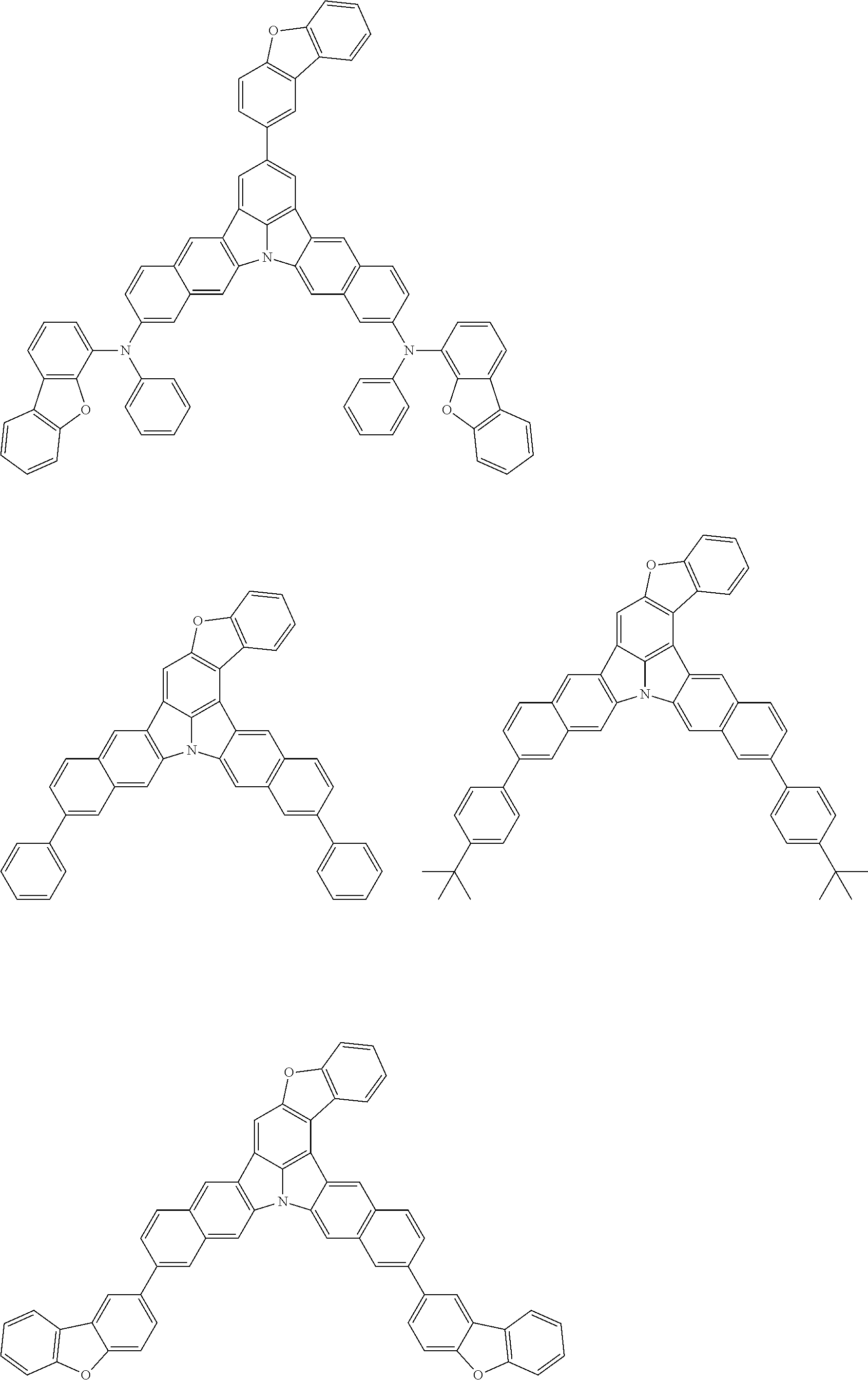

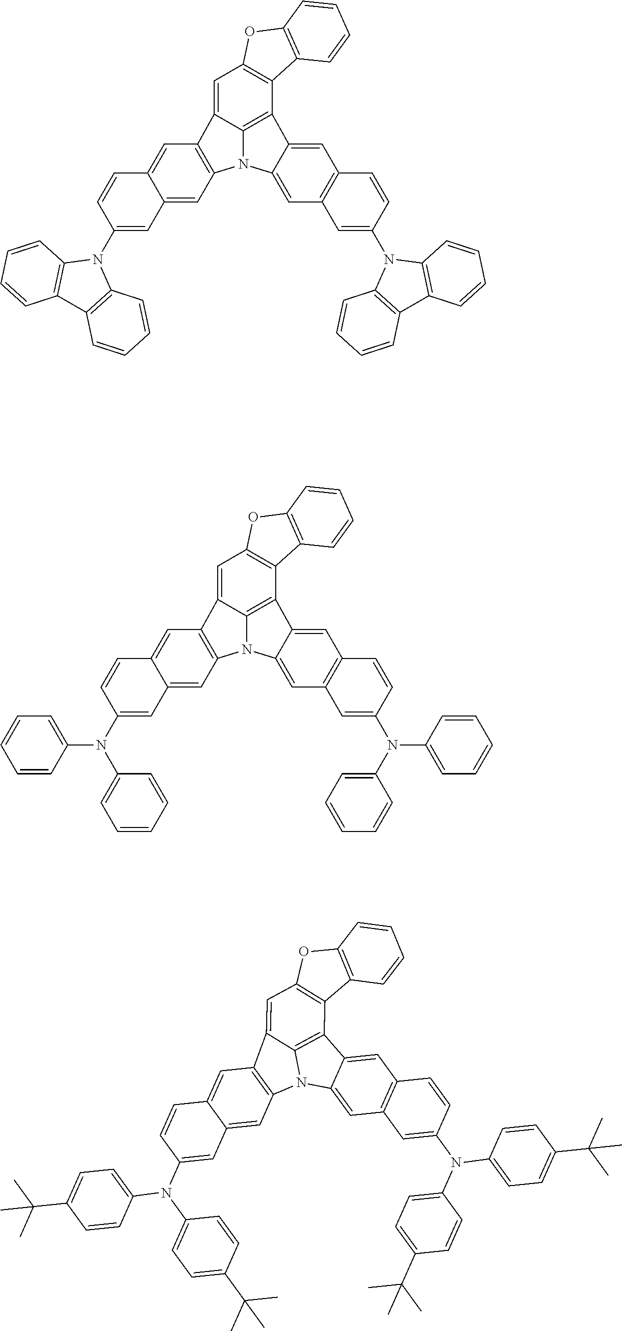 [SMILES: c1ccc2c(c1)oc1cc3c4cc5ccc(-n6c7ccccc7c7ccccc76)cc5cc4n4c5cc6cc(-n7c8ccccc8c8ccccc87)ccc6cc5c(c12)c34 ; c1ccc(N(c2ccccc2)c2ccc3cc4c5cc6oc7ccccc7c6c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6oc7ccccc7c6c6c7cc8ccc(N(c9ccc(C(C)(C)C)cc9)c9ccc(C(C)(C)C)cc9)cc8cc7n(c4cc3c2)c56)cc1]

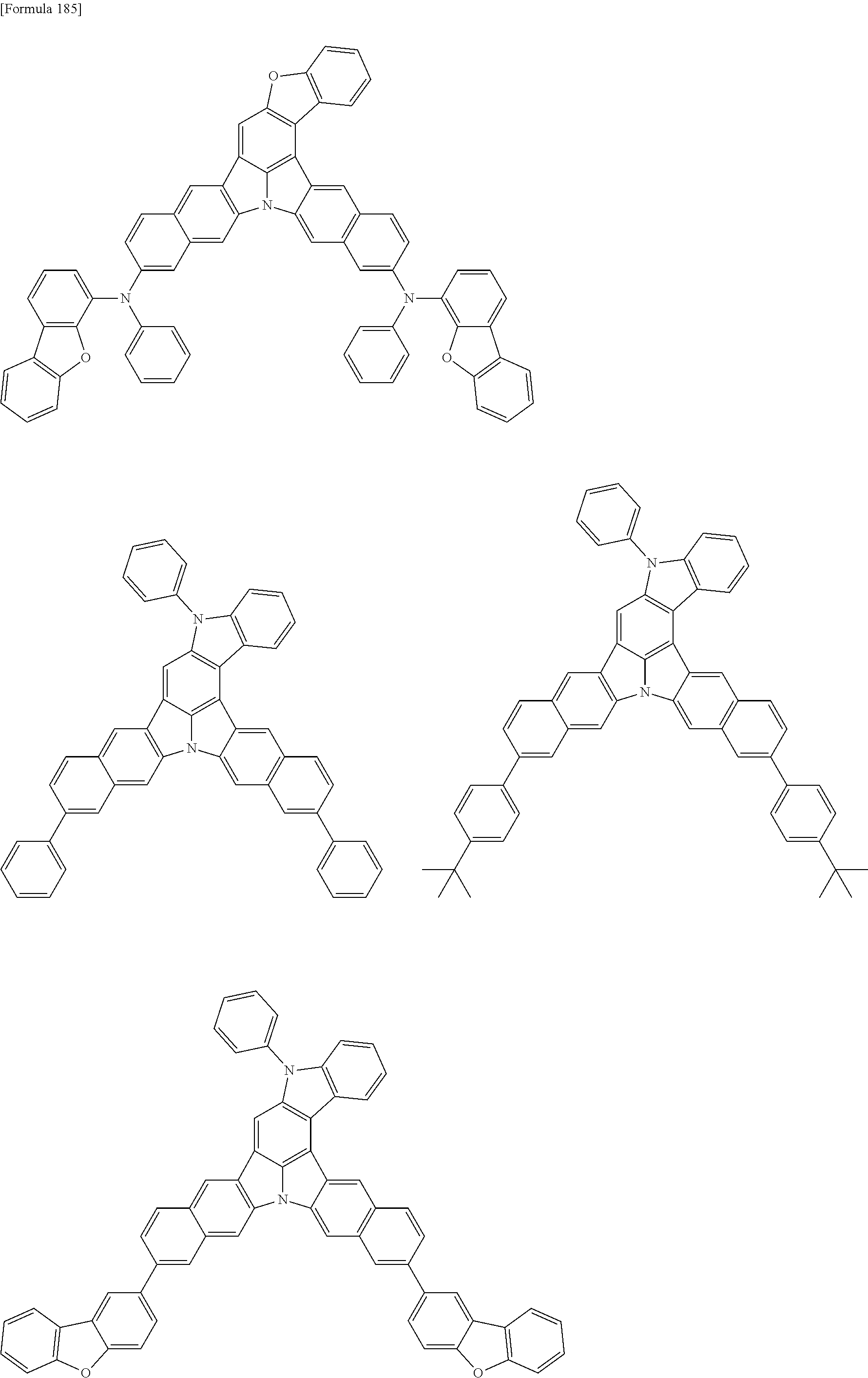

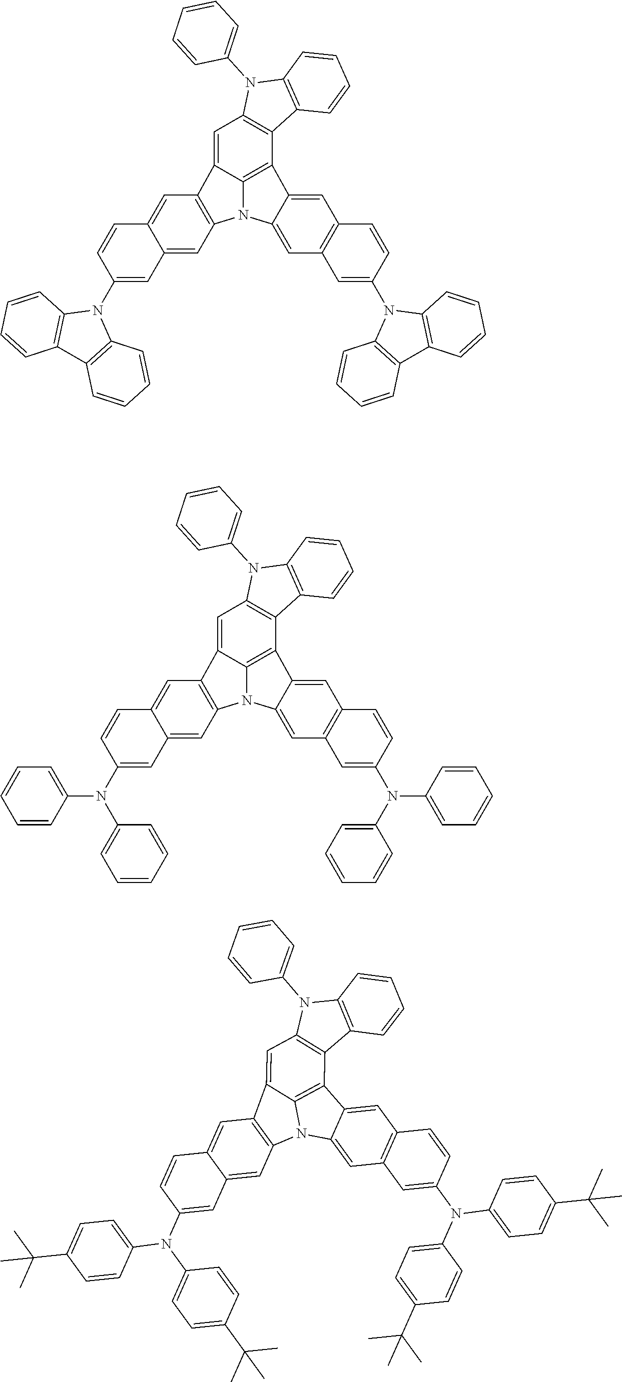 [SMILES: c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1 ; c1ccc(N(c2ccccc2)c2ccc3cc4c5cc6c(c7ccccc7n6-c6ccccc6)c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6c(c7ccccc7n6-c6ccccc6)c6c7cc8ccc(N(c9ccc(C(C)(C)C)cc9)c9ccc(C(C)(C)C)cc9)cc8cc7n(c4cc3c2)c56)cc1]

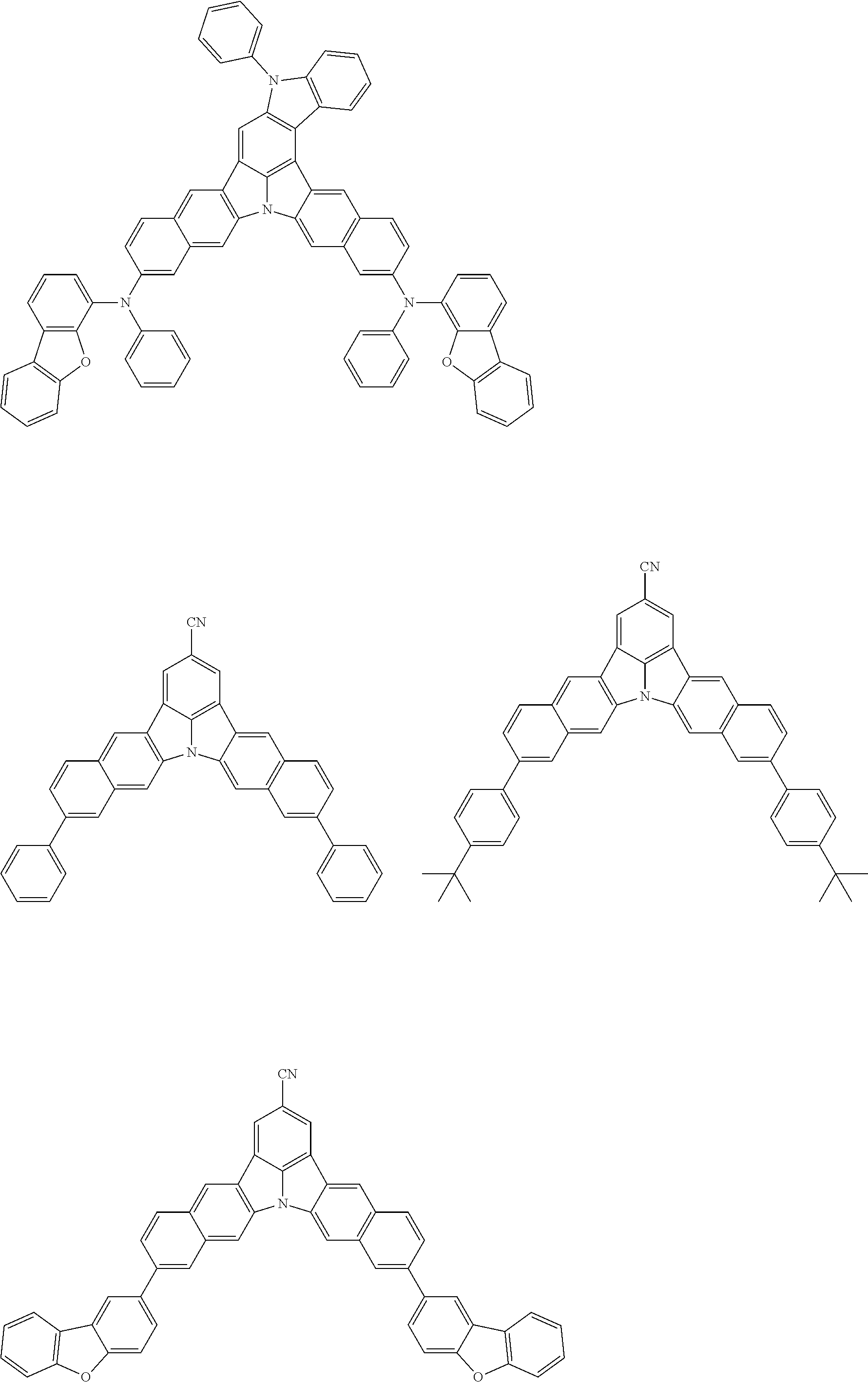

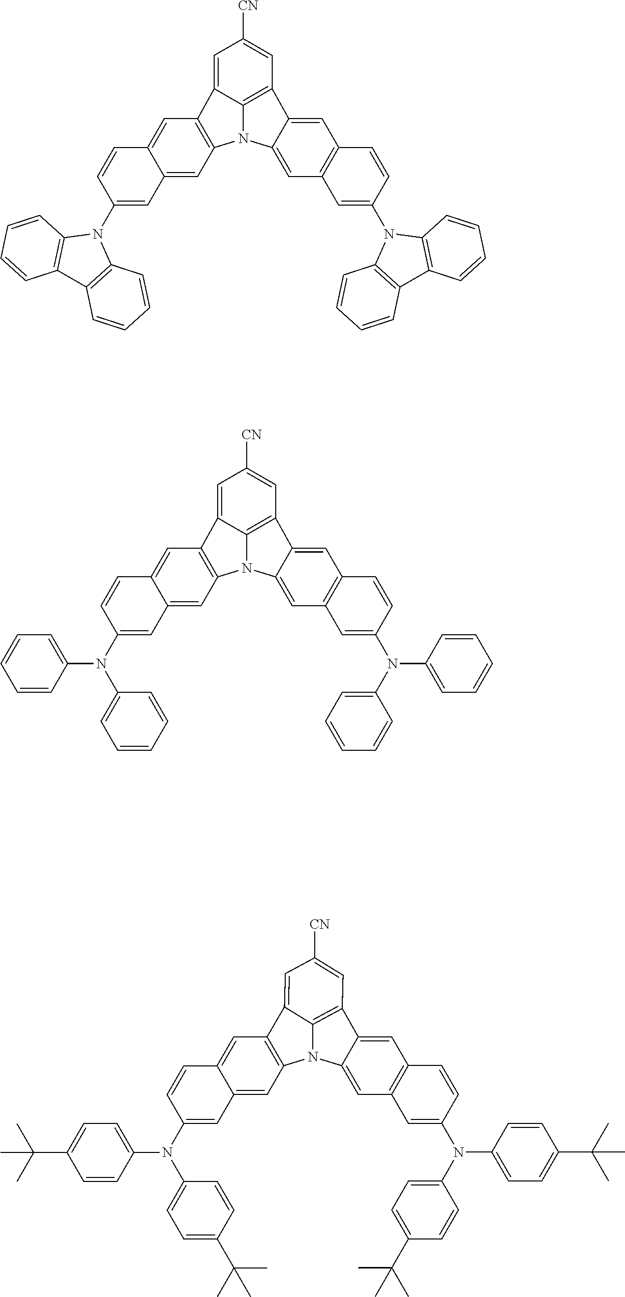 [SMILES: N#Cc1cc2c3cc4ccc(-n5c6ccccc6c6ccccc65)cc4cc3n3c4cc5cc(-n6c7ccccc7c7ccccc76)ccc5cc4c(c1)c23 ; N#Cc1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C#N)cc6c7cc8ccc(N(c9ccc(C(C)(C)C)cc9)c9ccc(C(C)(C)C)cc9)cc8cc7n(c4cc3c2)c56)cc1]

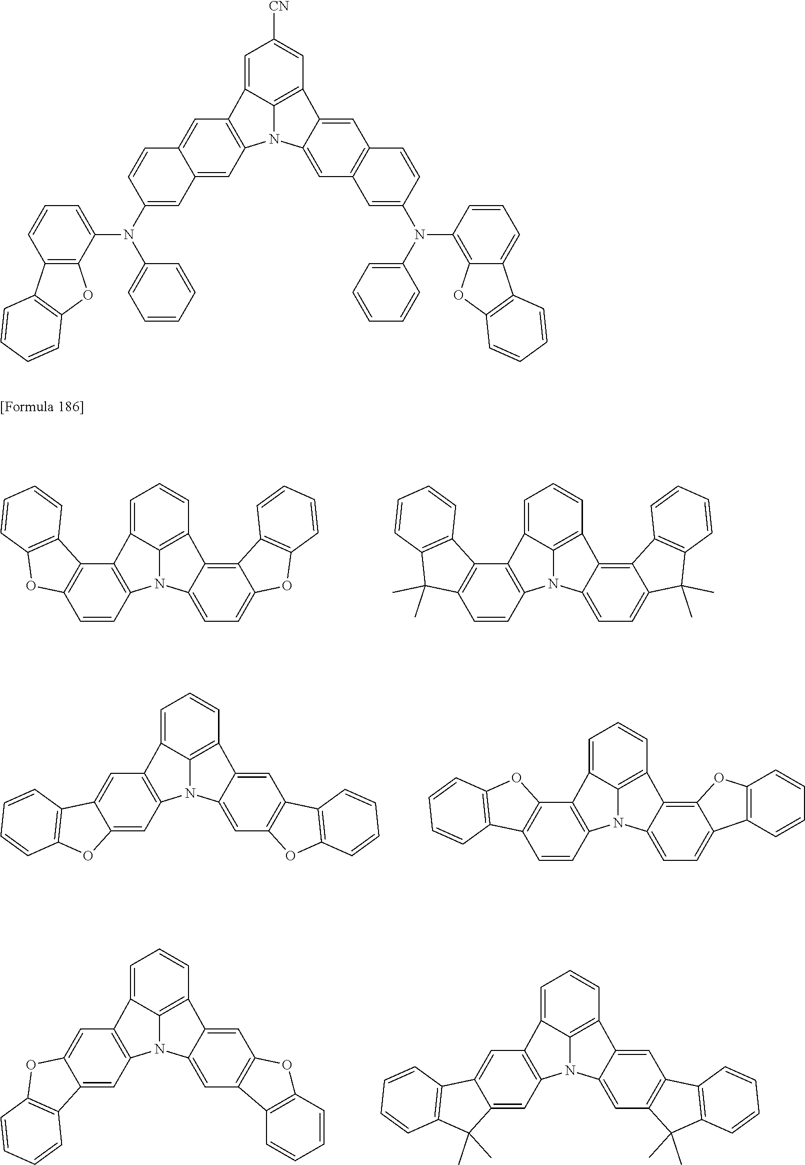

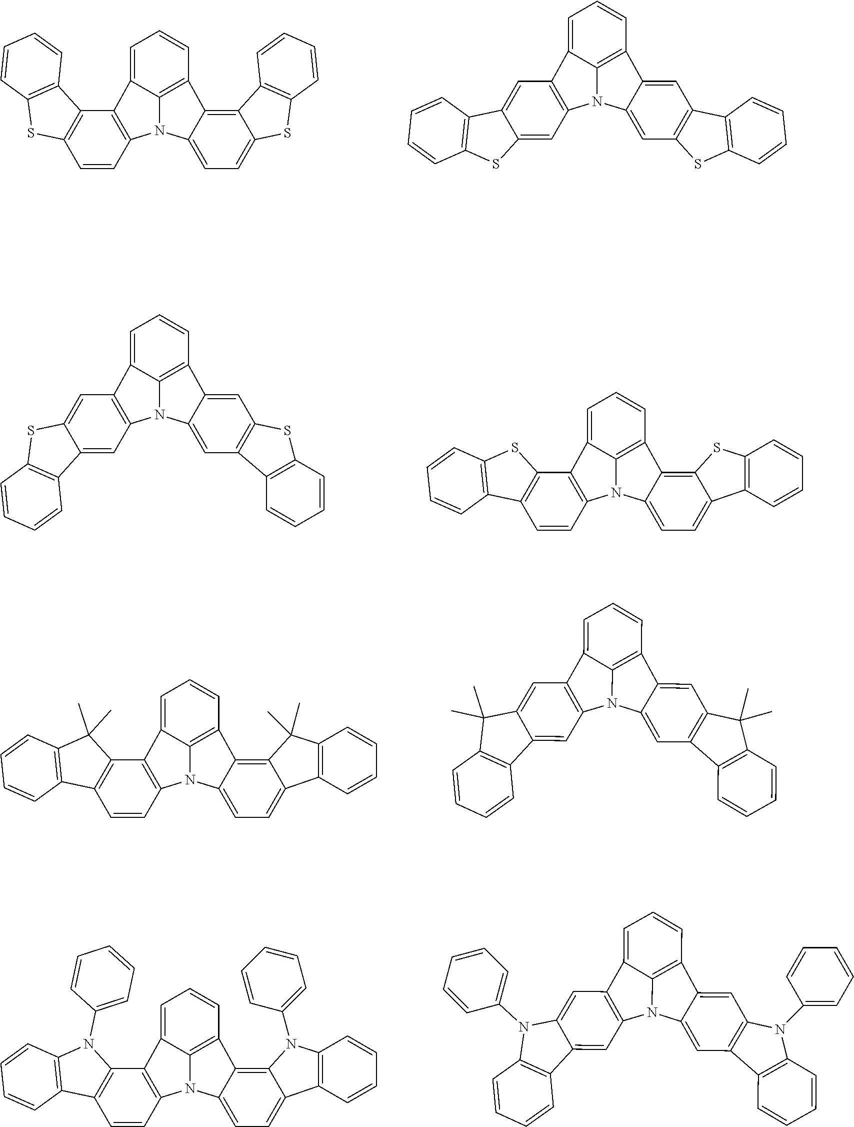

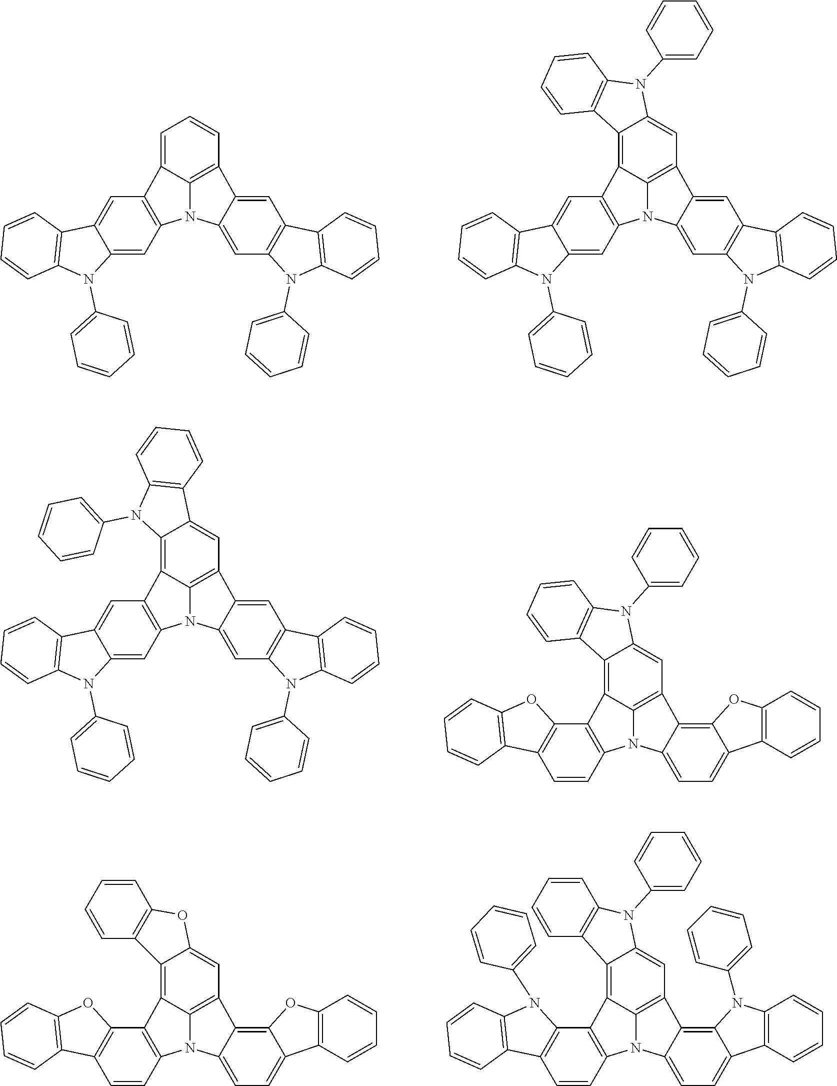

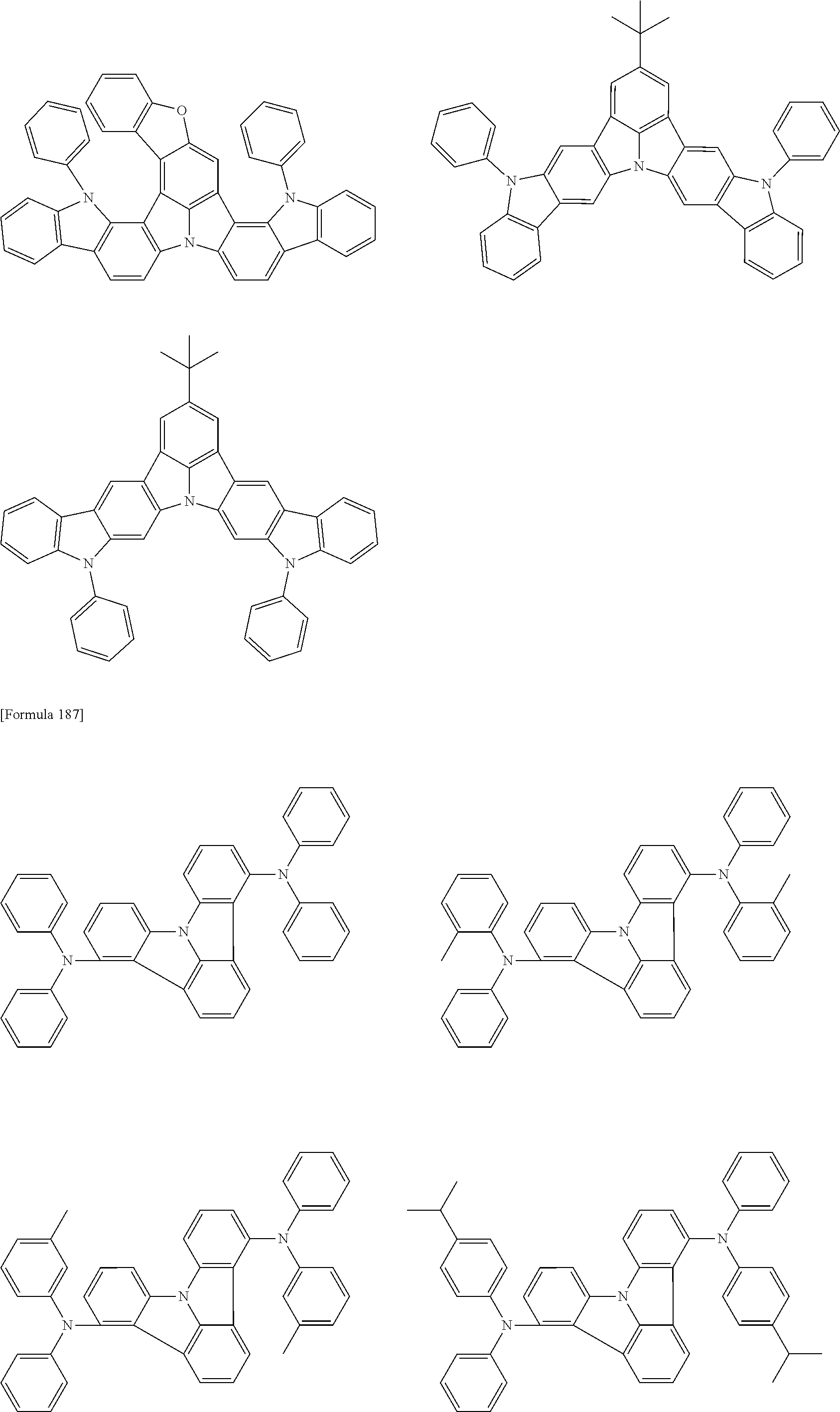

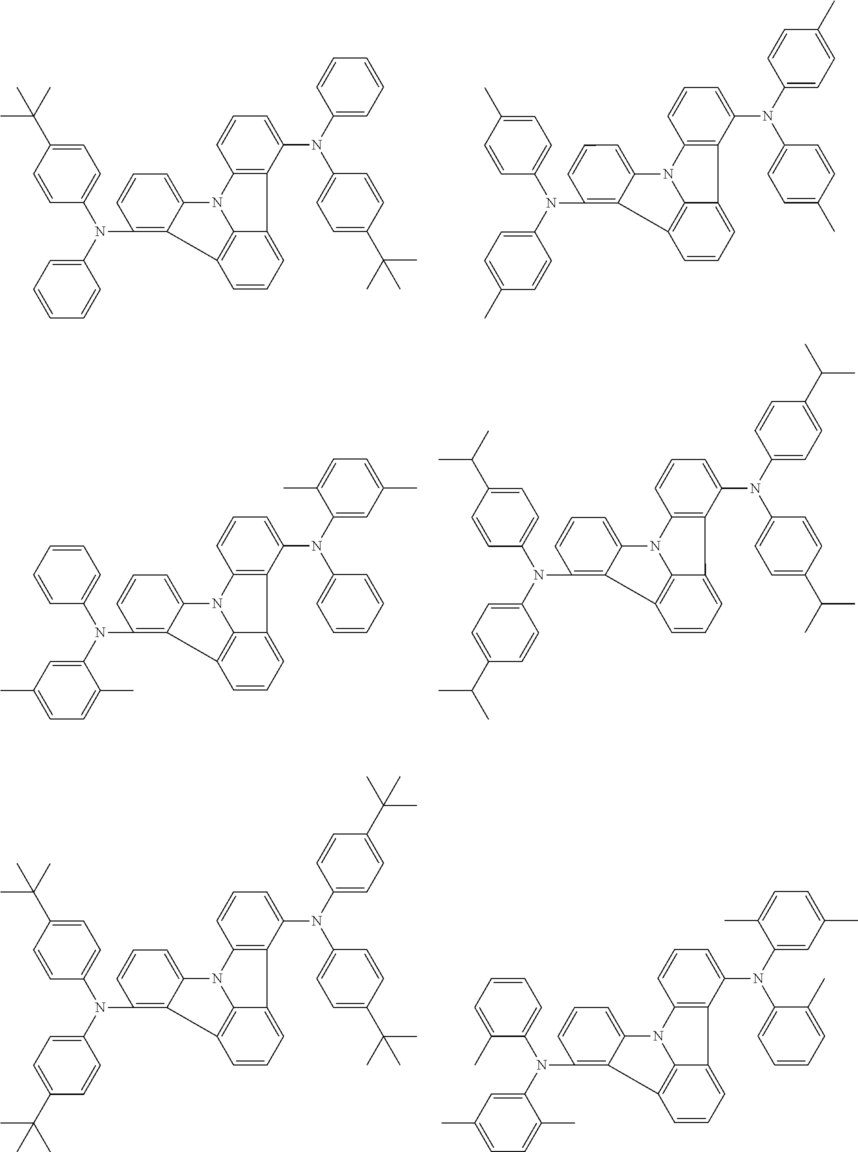

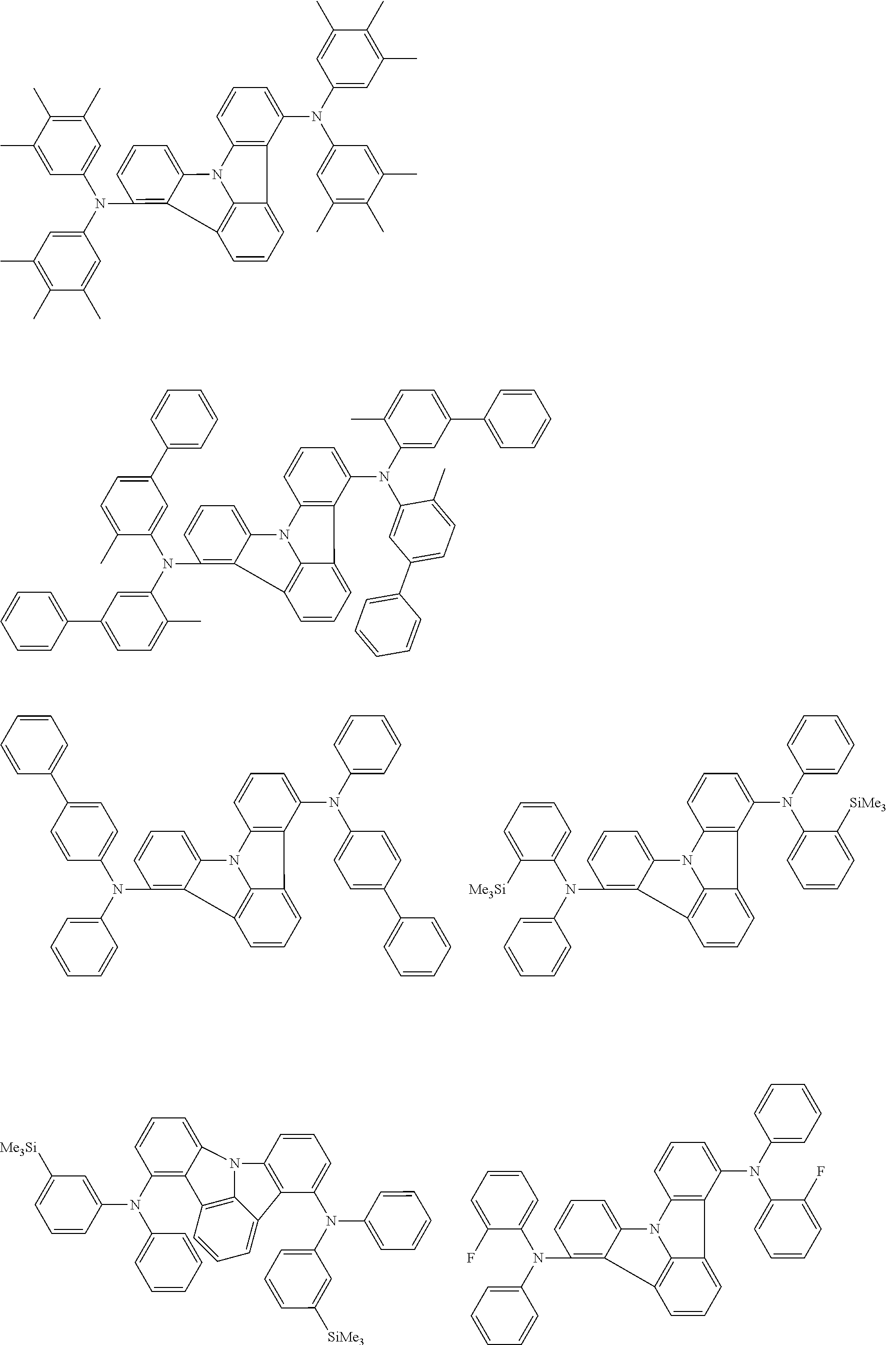

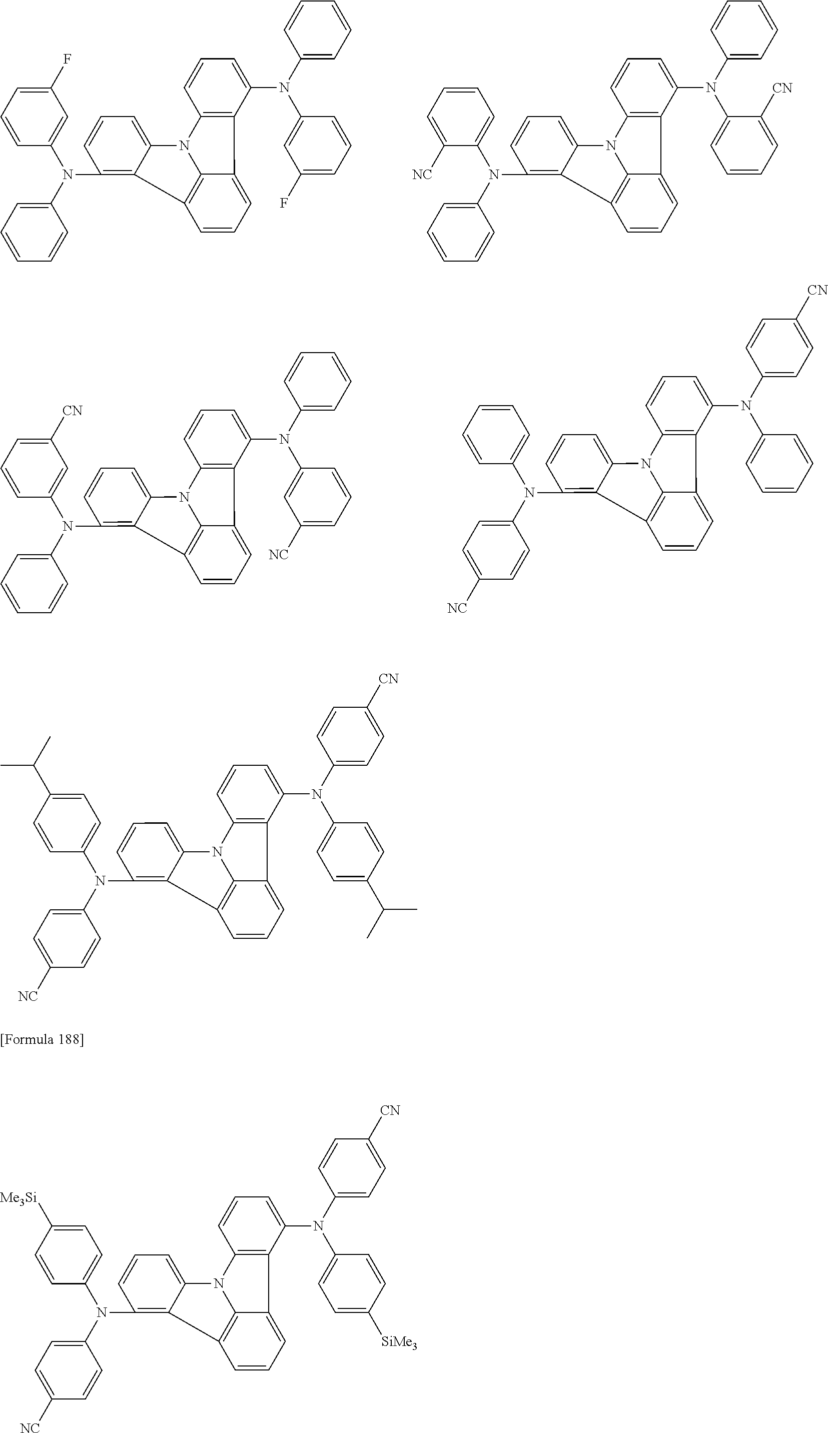

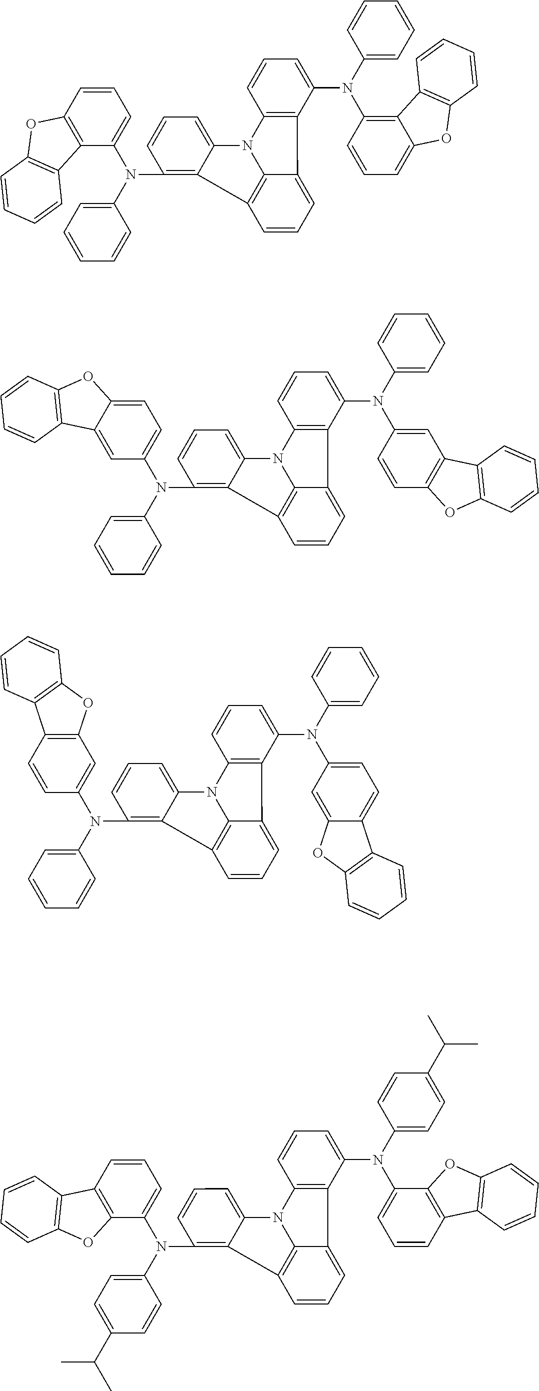 [SMILES: c1ccc(N(c2cccc3oc4ccccc4c23)c2cccc3c2c2cccc4c5c(N(c6ccccc6)c6cccc7oc8ccccc8c67)cccc5n3c24)cc1 ; c1ccc(N(c2ccc3oc4ccccc4c3c2)c2cccc3c2c2cccc4c5c(N(c6ccccc6)c6ccc7oc8ccccc8c7c6)cccc5n3c24)cc1 ; c1ccc(N(c2ccc3c(c2)oc2ccccc23)c2cccc3c2c2cccc4c5c(N(c6ccccc6)c6ccc7c(c6)oc6ccccc67)cccc5n3c24)cc1 ; CC(C)c1ccc(N(c2cccc3c2oc2ccccc23)c2cccc3c2c2cccc4c5c(N(c6ccc(C(C)C)cc6)c6cccc7c6oc6ccccc67)cccc5n3c24)cc1]

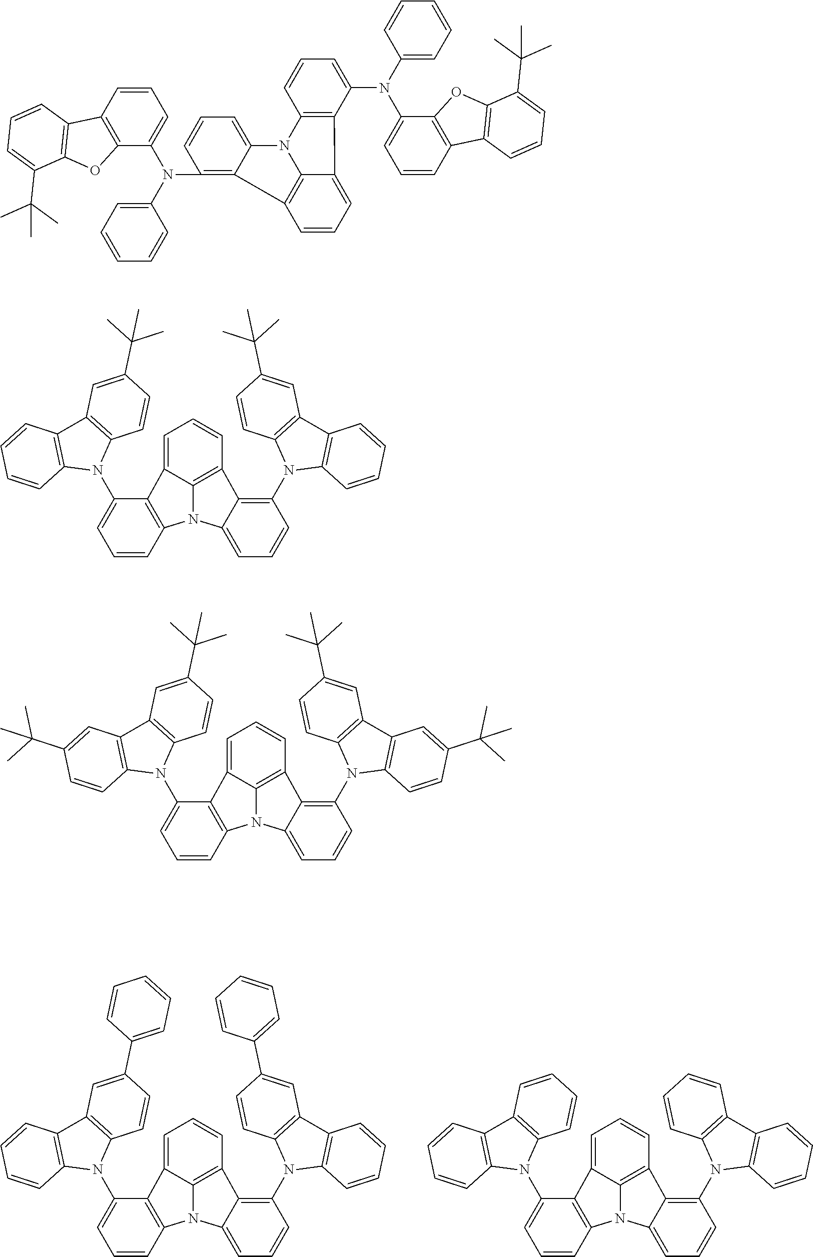

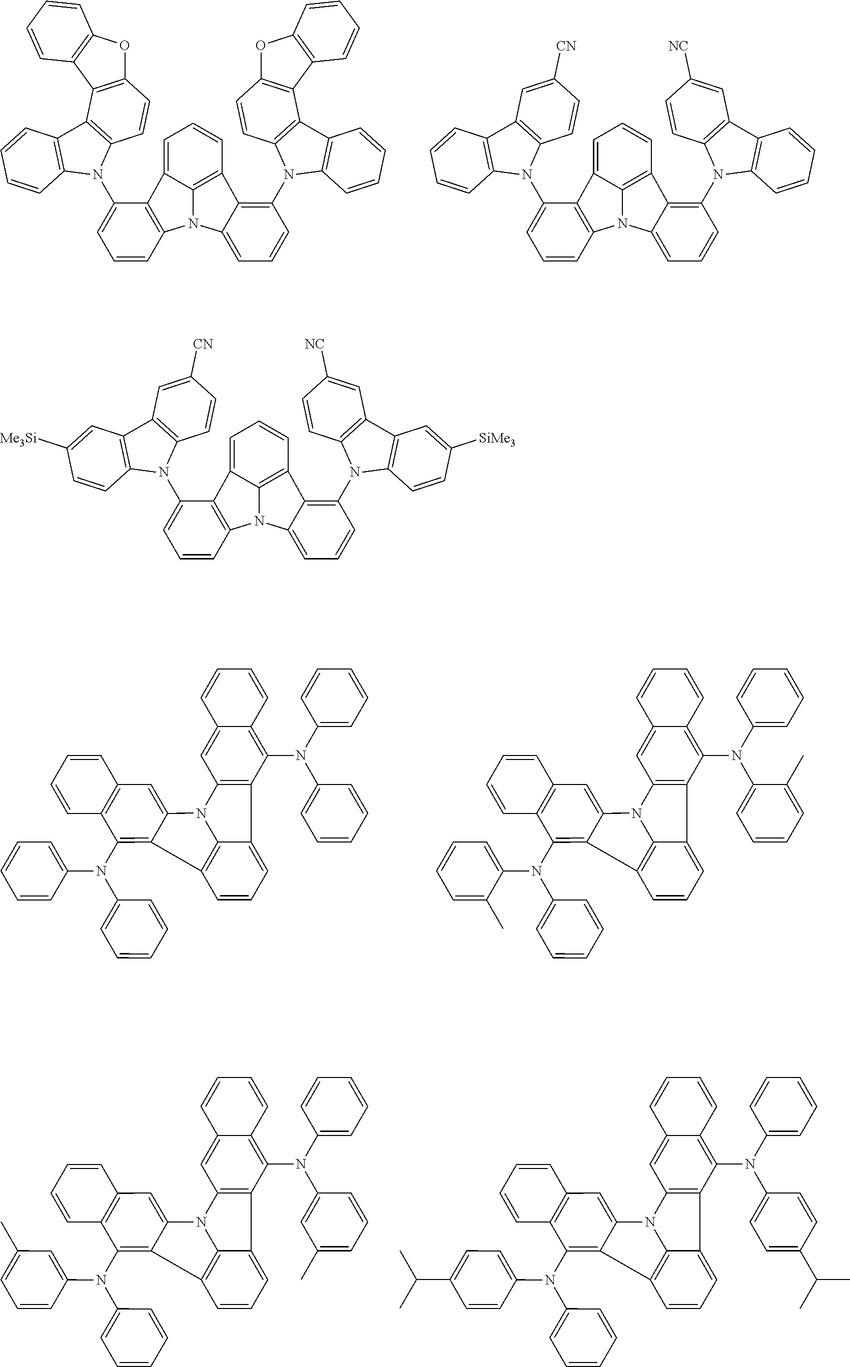

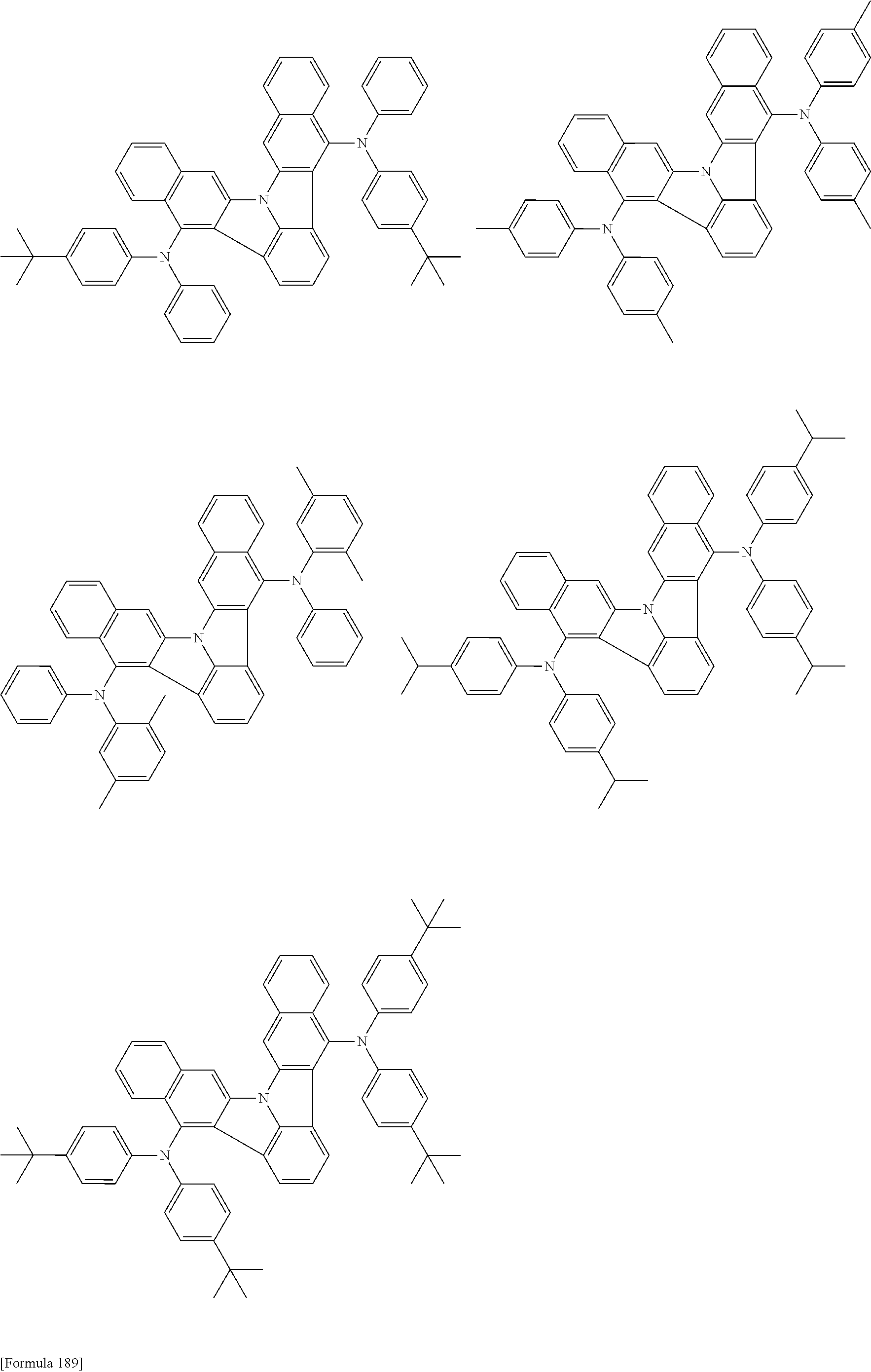

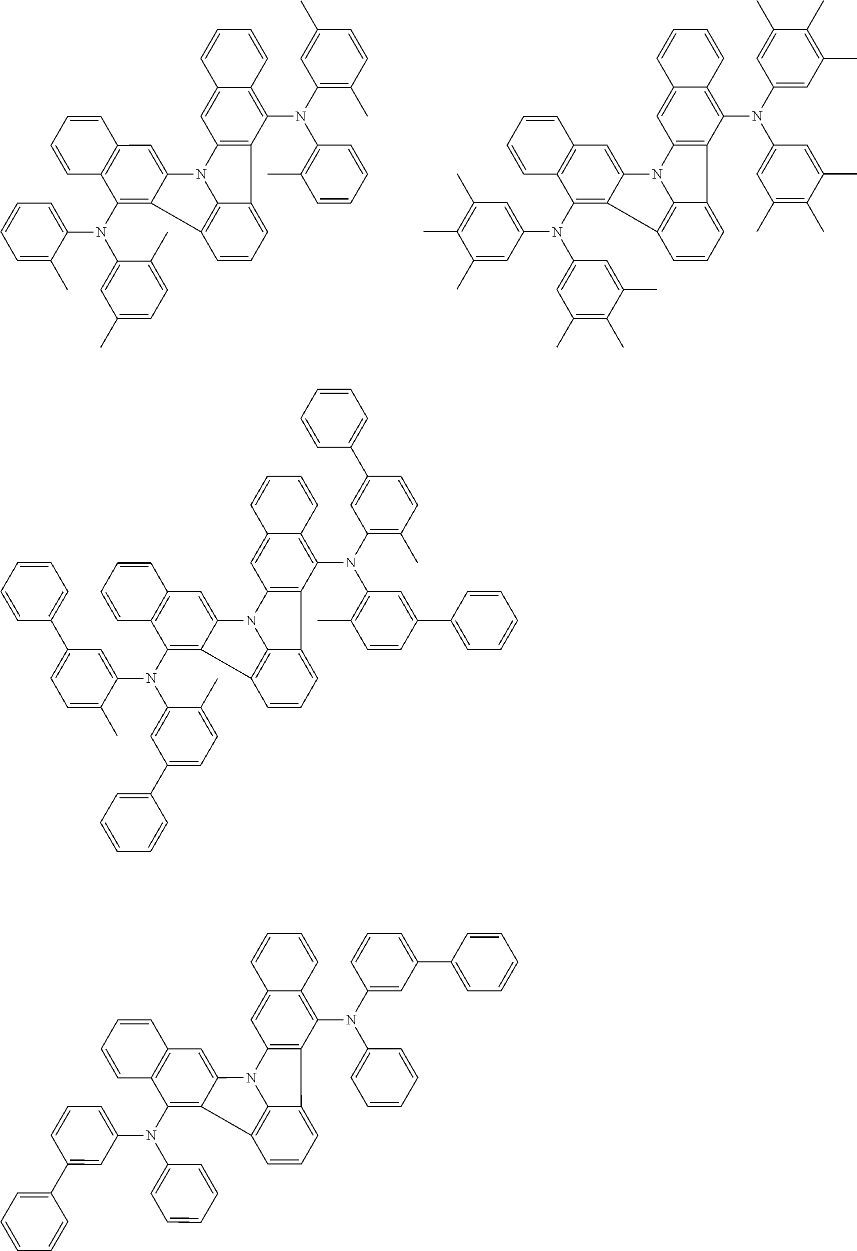

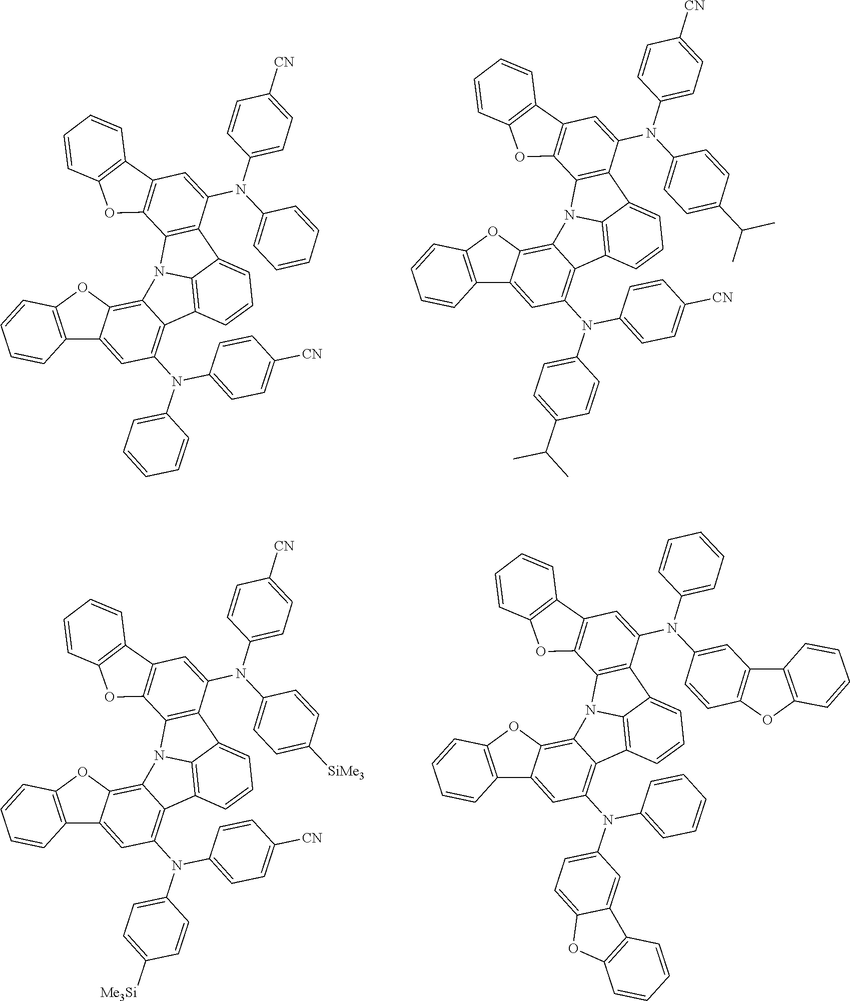 [SMILES: N#Cc1ccc(N(c2ccccc2)c2cc3c4ccccc4oc3c3c2c2cccc4c5c(N(c6ccccc6)c6ccc(C#N)cc6)cc6c7ccccc7oc6c5n3c24)cc1 ; CC(C)c1ccc(N(c2ccc(C#N)cc2)c2cc3c4ccccc4oc3c3c2c2cccc4c5c(N(c6ccc(C#N)cc6)c6ccc(C(C)C)cc6)cc6c7ccccc7oc6c5n3c24)cc1 ; C[Si](C)(C)c1ccc(N(c2ccc(C#N)cc2)c2cc3c4ccccc4oc3c3c2c2cccc4c5c(N(c6ccc(C#N)cc6)c6ccc([Si](C)(C)C)cc6)cc6c7ccccc7oc6c5n3c24)cc1 ; c1ccc(N(c2ccc3oc4ccccc4c3c2)c2cc3c4ccccc4oc3c3c2c2cccc4c5c(N(c6ccccc6)c6ccc7oc8ccccc8c7c6)cc6c7ccccc7oc6c5n3c24)cc1]

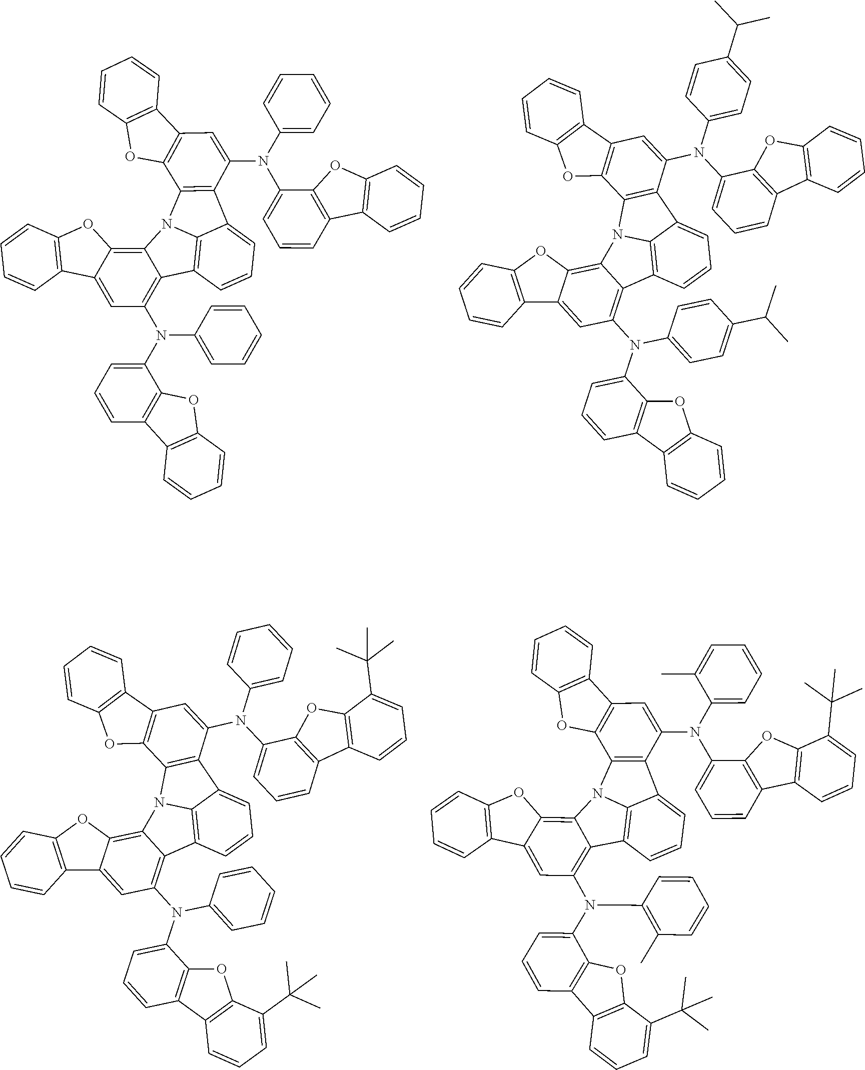

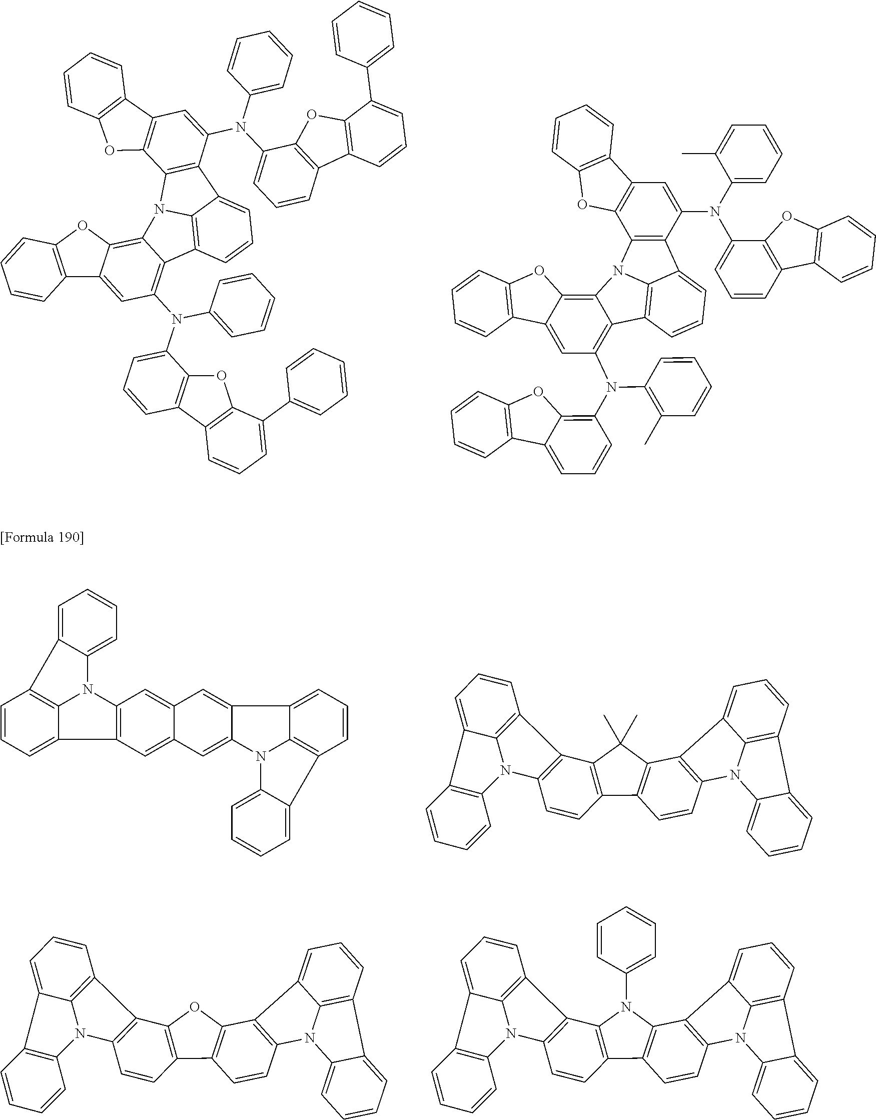

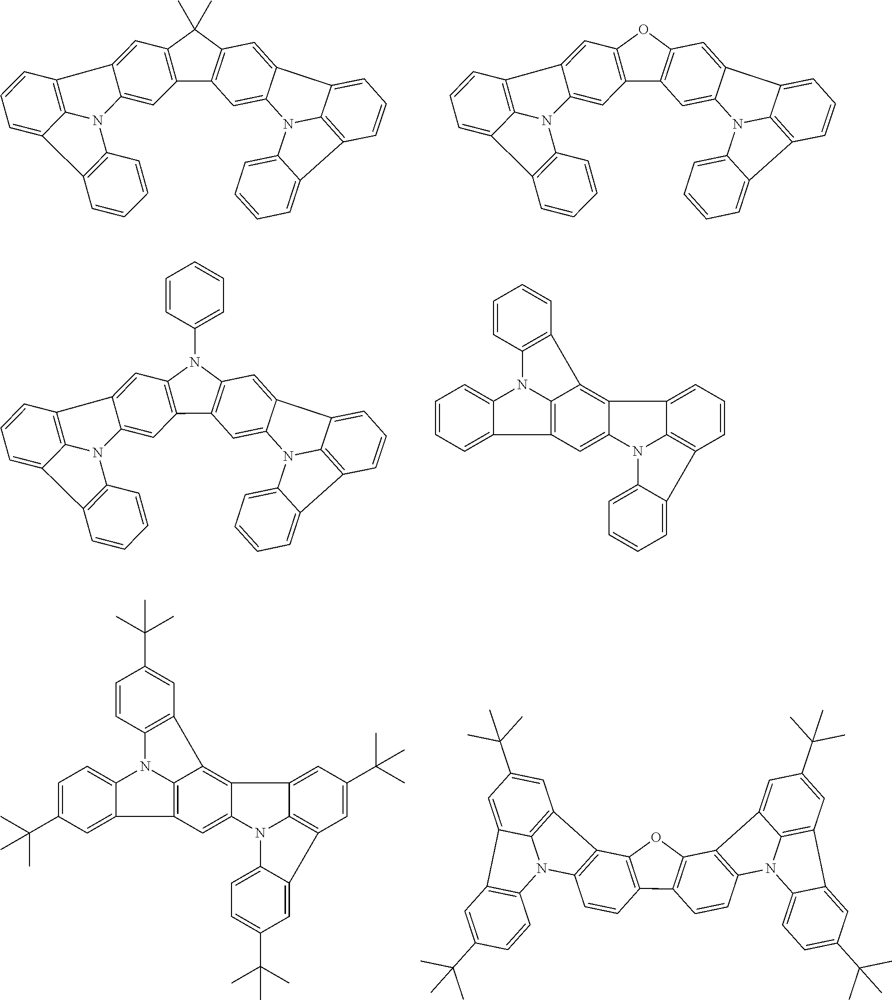 [SMILES: CC1(C)c2cc3c4cccc5c6ccccc6n(c3cc2-c2cc3c(cc21)c1cccc2c6ccccc6n3c21)c54 ; c1ccc2c(c1)c1cccc3c4cc5oc6cc7c8cccc9c%10ccccc%10n(c7cc6c5cc4n2c13)c98 ; c1ccc(-n2c3cc4c5cccc6c7ccccc7n(c4cc3c3cc4c(cc32)c2cccc3c7ccccc7n4c32)c65)cc1 ; c1ccc2c(c1)c1cccc3c4c5c6ccccc6n6c7ccccc7c(cc4n2c13)c56 ; CC(C)(C)c1ccc2c(c1)c1cc(C(C)(C)C)cc3c4c5c6cc(C(C)(C)C)ccc6n6c7ccc(C(C)(C)C)cc7c(cc4n2c13)c56 ; CC(C)(C)c1ccc2c(c1)c1cc(C(C)(C)C)cc3c4c5oc6c(ccc7c6c6cc(C(C)(C)C)cc8c9cc(C(C)(C)C)ccc9n7c86)c5ccc4n2c13]

Compound Represented by Formula (5)

A compound represented by the formula (5) is described below. A compound represented by the formula (5) is a compound corresponding to a compound represented by the formula (41-3).

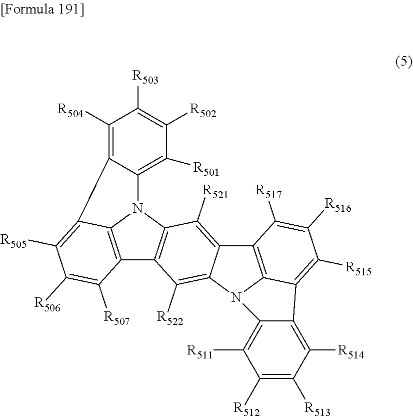

In the formula (5),
at least one combination of adjacent two or more of R₅₀₁ to R₅₀₇ and R₅₁₁ to R₅₁₇ are mutually bonded to form a substituted or unsubstituted monocyclic ring, are mutually bonded to form a substituted or unsubstituted fused ring, or are not mutually bonded,
R₅₀₁ to R₅₀₇ and R₅₁₁ to R₅₁₇ that do not form the monocyclic ring and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.
R₅₂₁ and R₅₂₂ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

“A combination of adjacent two or more of R₅₀₁ to R₅₀₇ and R₅₁₁ to R₅₁₇” is a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, or a combination of R₅₀₁, R₅₀₂, and R₅₀₃, for example.

In an exemplary embodiment, at least one, preferably two, of R₅₀₁ to R₅₀₇ or R₅₁₁ to R₅₁₇ is a group represented by —N(R₉₀₆)(R₉₀₇).

In an exemplary embodiment, R₅₀₁ to R₅₀₇ and R₅₁₁ to R₅₁₇ each independently represent a hydrogen atom, a substituted or unsubstituted aryl group having 6 to 50 ring carbon atoms, or a substituted or unsubstituted heterocyclic group having 5 to 50 ring atoms.

In an exemplary embodiment, a compound represented by the formula (5) is a compound represented by the following formula (52).

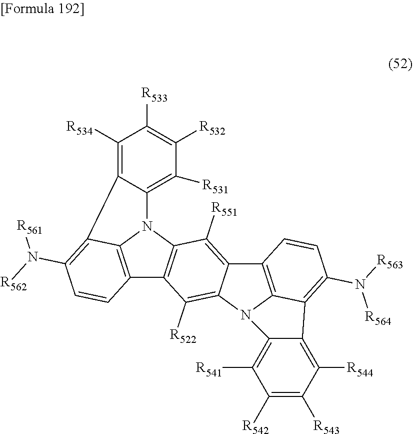

In the formula (52),
at least one combination of adjacent two or more of R₅₃₁ to R₅₃₄ and R₅₄₁ to R₅₄₄ are mutually bonded to form a substituted or unsubstituted monocyclic ring, are mutually bonded to form a substituted or unsubstituted fused ring, or are not mutually bonded,
R₅₃₁ to R₅₃₄, R₅₄₁ to R₅₄₄, and R₅₅₁ and R₅₅₂ that do not form the monocyclic ring and do not form the fused ring each independently represent a hydrogen atom, a substituted or unsubstituted aryl group having 6 to 50 ring carbon atoms, or a substituted or unsubstituted heterocyclic group having 5 to 50 ring atoms, and
R₅₆₁ to R₅₆₄ each independently represent a substituted or unsubstituted aryl group having 6 to 50 ring carbon atoms, or a substituted or unsubstituted heterocyclic group having 5 to 50 ring atoms.

In an exemplary embodiment, a compound represented by the formula (5) is a compound represented by the following formula (53).

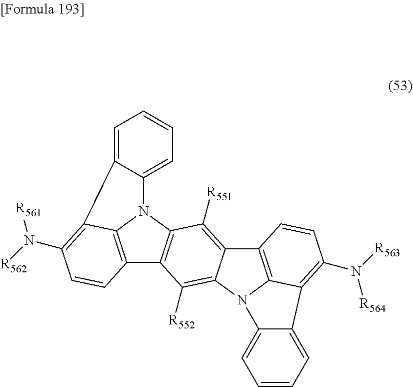

In the formula (53), R₅₅₁, R₅₅₂, and R₅₆₁ to R₅₆₄ represent the same as R₅₅₁, R₅₅₂, and R₅₆₁ to R₅₆₄, respectively, in the formula (52).

In an exemplary embodiment, R₅₆₁ to R₅₆₄ in the formula (52) and the formula (53) each independently represent a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formula (52) and the formula (53) are hydrogen atoms.

In an exemplary embodiment, a substituent for “substituted or unsubstituted” in the formula (5), the formula (52), and the formula (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

Specific examples of a compound represented by the formula (5) include the following compounds.

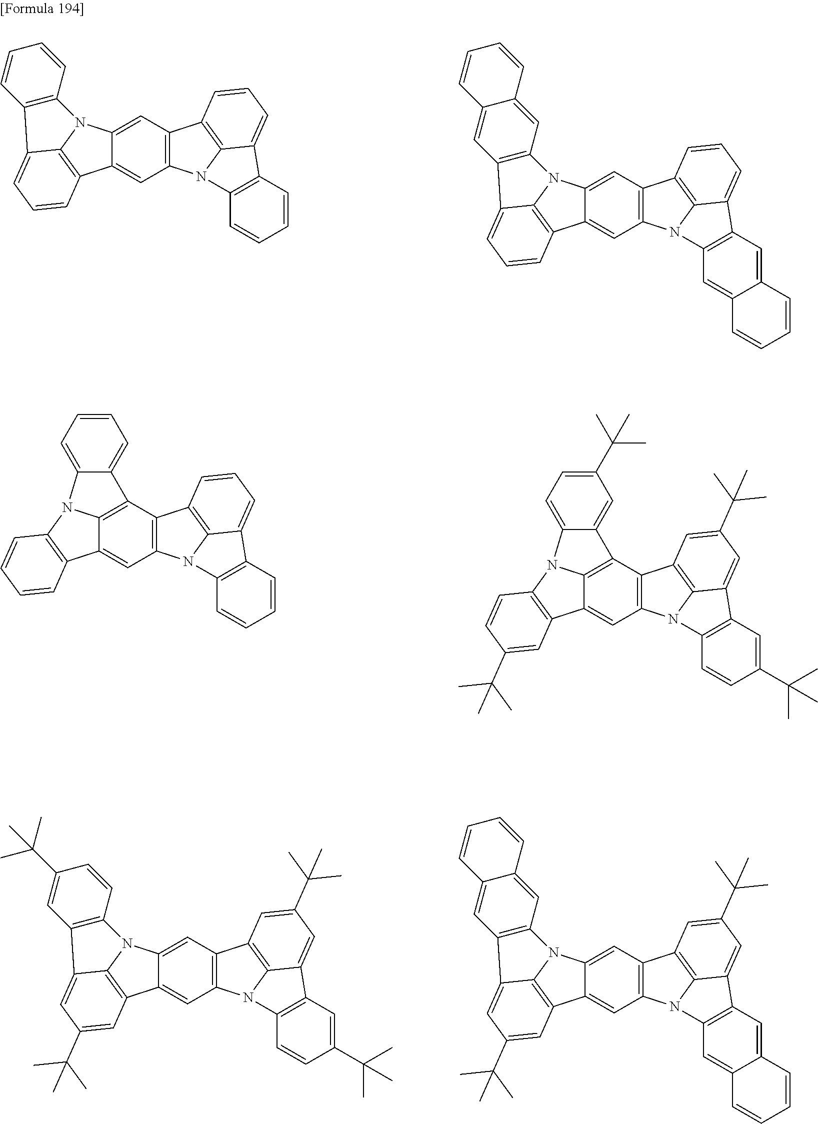 [SMILES: c1ccc2c(c1)c1cccc3c4cc5c(cc4n2c13)c1cccc2c3ccccc3n5c21 ; c1ccc2cc3c(cc2c1)c1cccc2c4cc5c(cc4n3c12)c1cccc2c3cc4ccccc4cc3n5c21 ; c1ccc2c(c1)c1cccc3c4c5c6ccccc6n6c7ccccc7c(cc4n2c13)c56 ; CC(C)(C)c1ccc2c(c1)c1cc(C(C)(C)C)cc3c4c5c6cc(C(C)(C)C)ccc6n6c7ccc(C(C)(C)C)cc7c(cc4n2c13)c56 ; CC(C)(C)c1ccc2c(c1)c1cc(C(C)(C)C)cc3c4cc5c(cc4n2c13)c1cc(C(C)(C)C)cc2c3cc(C(C)(C)C)ccc3n5c21 ; CC(C)(C)c1cc2c3cc4ccccc4cc3n3c4cc5c6cc(C(C)(C)C)cc7c8cc9ccccc9cc8n(c5cc4c(c1)c23)c76]

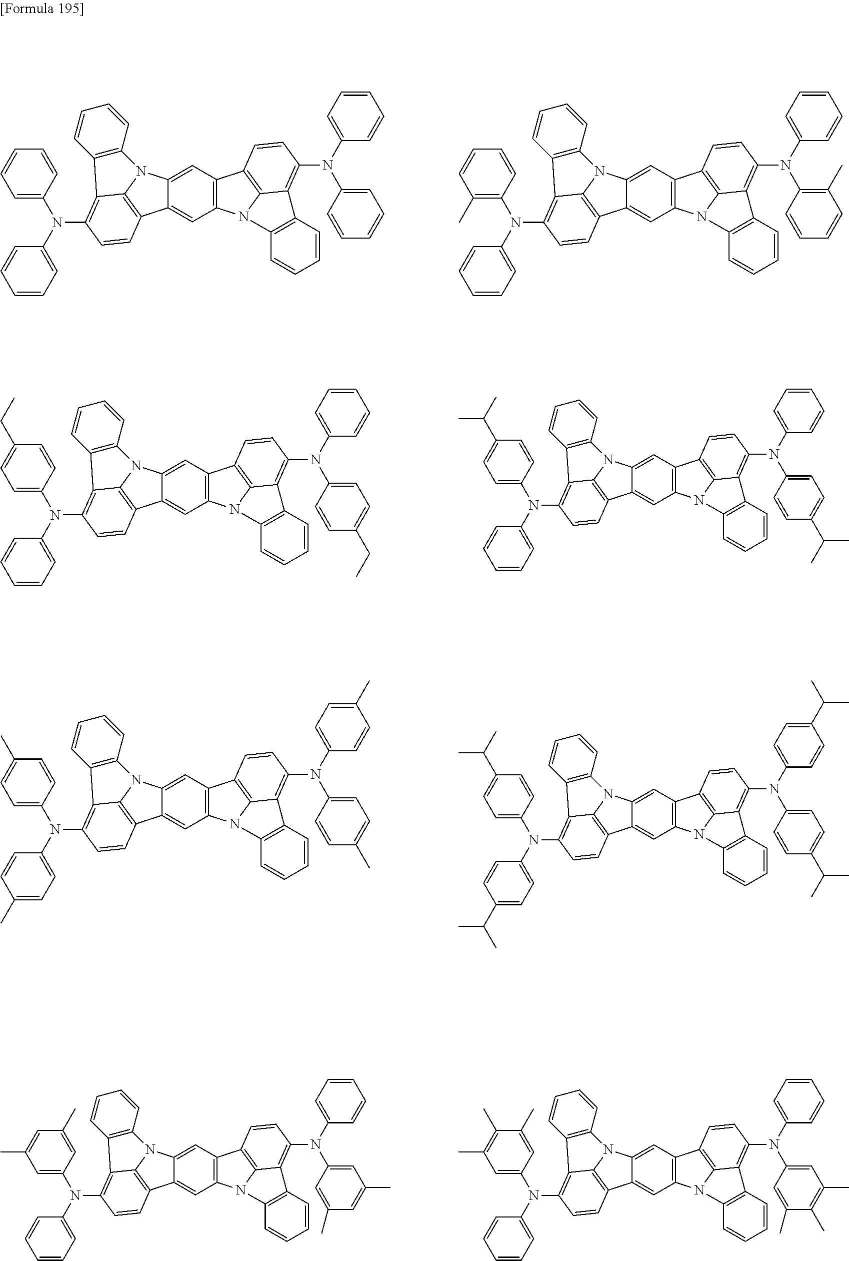

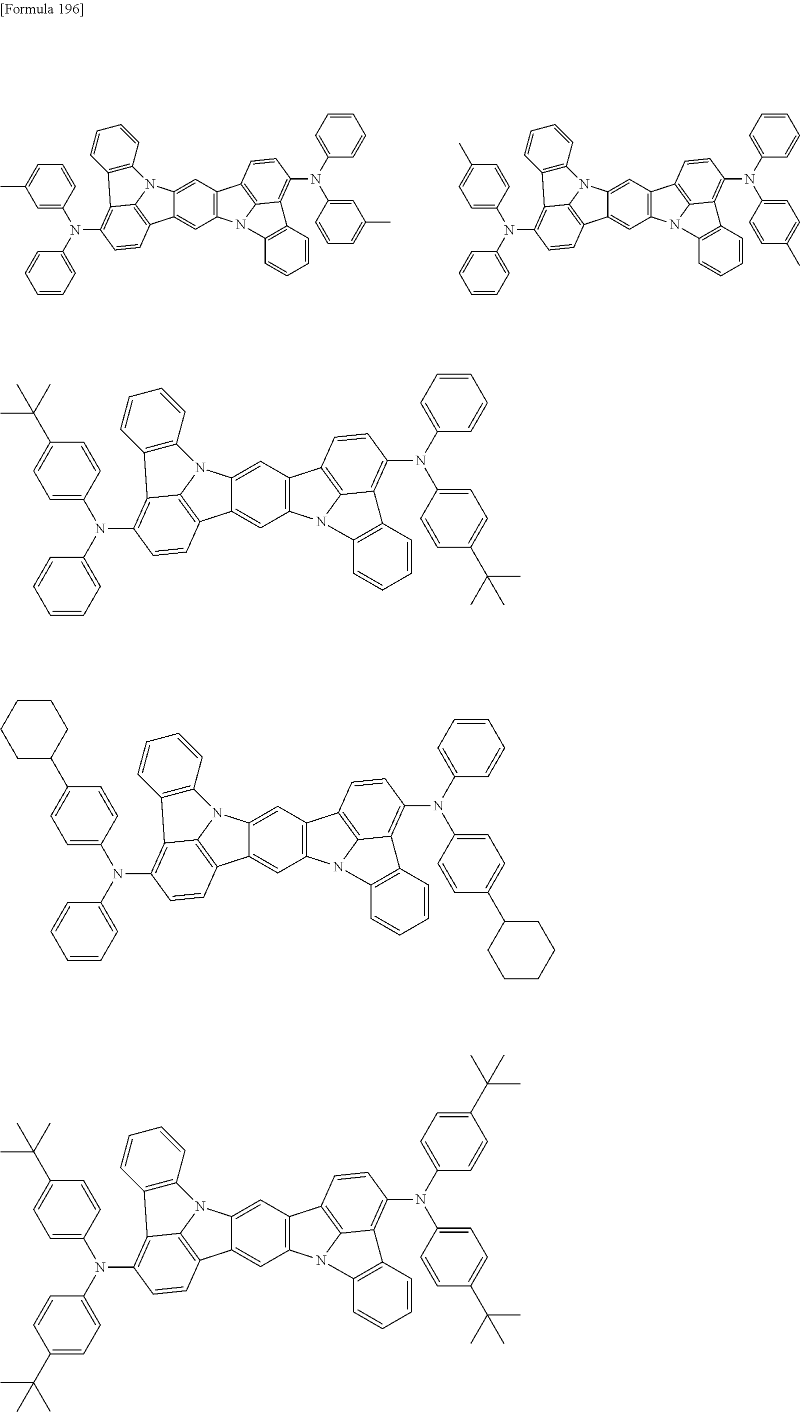

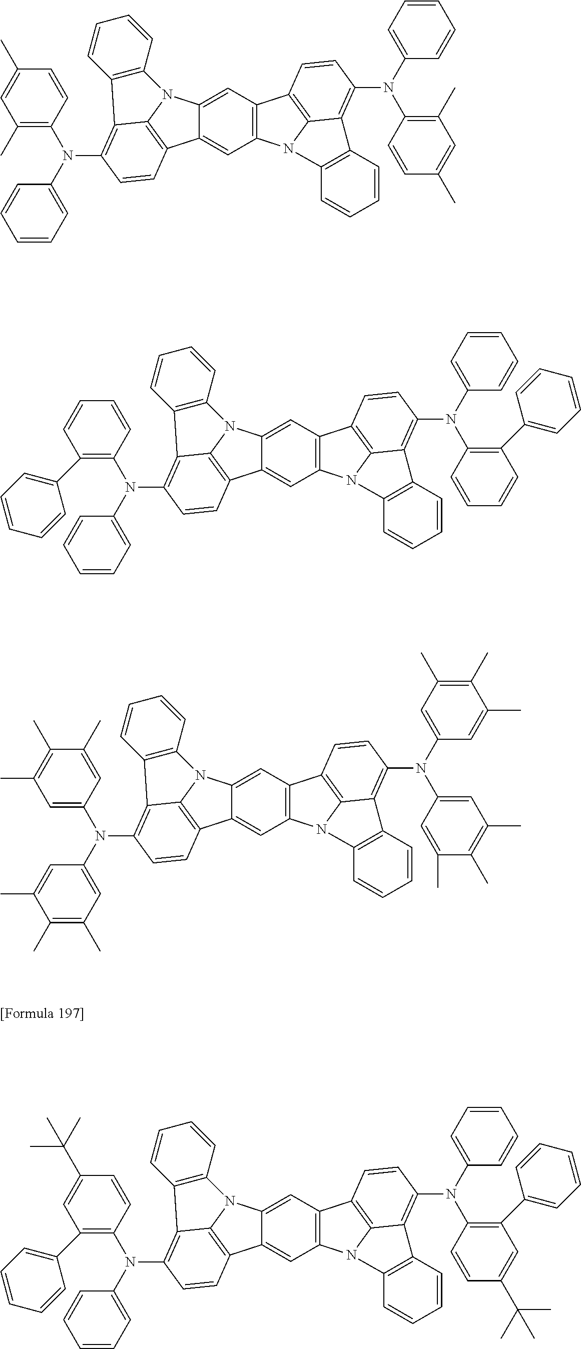

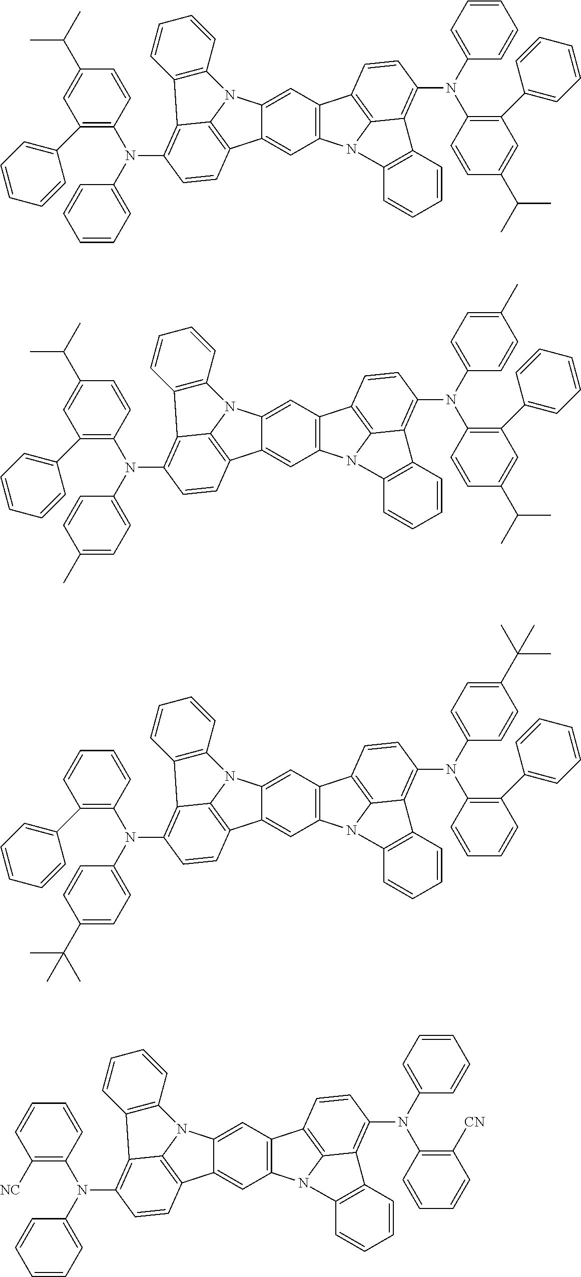 [SMILES: CC(C)c1ccc(N(c2ccccc2)c2ccc3c4cc5c(cc4n4c6ccccc6c2c34)c2ccc(N(c3ccccc3)c3ccc(C(C)C)cc3-c3ccccc3)c3c4ccccc4n5c23)c(-c2ccccc2)c1 ; Cc1ccc(N(c2ccc(C(C)C)cc2-c2ccccc2)c2ccc3c4cc5c(cc4n4c6ccccc6c2c34)c2ccc(N(c3ccc(C)cc3)c3ccc(C(C)C)cc3-c3ccccc3)c3c4ccccc4n5c23)cc1 ; CC(C)(C)c1ccc(N(c2ccccc2-c2ccccc2)c2ccc3c4cc5c(cc4n4c6ccccc6c2c34)c2ccc(N(c3ccc(C(C)(C)C)cc3)c3ccccc3-c3ccccc3)c3c4ccccc4n5c23)cc1 ; N#Cc1ccccc1N(c1ccccc1)c1ccc2c3cc4c(cc3n3c5ccccc5c1c23)c1ccc(N(c2ccccc2)c2ccccc2C#N)c2c3ccccc3n4c12]

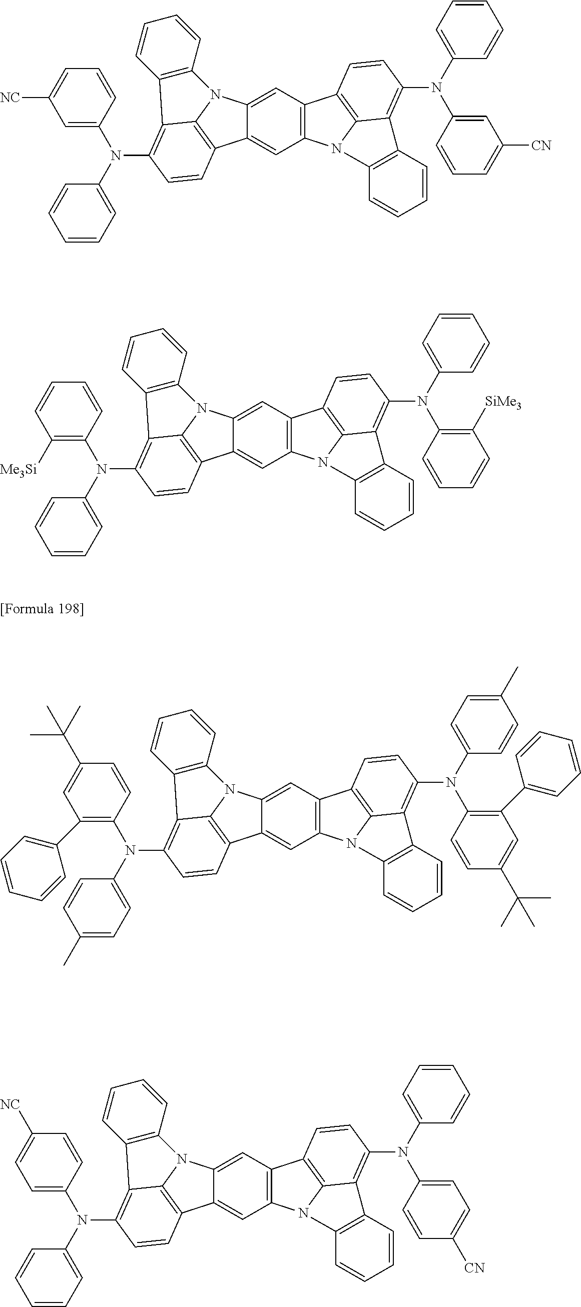 [SMILES: N#Cc1cccc(N(c2ccccc2)c2ccc3c4cc5c(cc4n4c6ccccc6c2c34)c2ccc(N(c3ccccc3)c3cccc(C#N)c3)c3c4ccccc4n5c23)c1 ; C[Si](C)(C)c1ccccc1N(c1ccccc1)c1ccc2c3cc4c(cc3n3c5ccccc5c1c23)c1ccc(N(c2ccccc2)c2ccccc2[Si](C)(C)C)c2c3ccccc3n4c12 ; Cc1ccc(N(c2ccc(C(C)(C)C)cc2-c2ccccc2)c2ccc3c4cc5c(cc4n4c6ccccc6c2c34)c2ccc(N(c3ccc(C)cc3)c3ccc(C(C)(C)C)cc3-c3ccccc3)c3c4ccccc4n5c23)cc1 ; N#Cc1ccc(N(c2ccccc2)c2ccc3c4cc5c(cc4n4c6ccccc6c2c34)c2ccc(N(c3ccccc3)c3ccc(C#N)cc3)c3c4ccccc4n5c23)cc1]

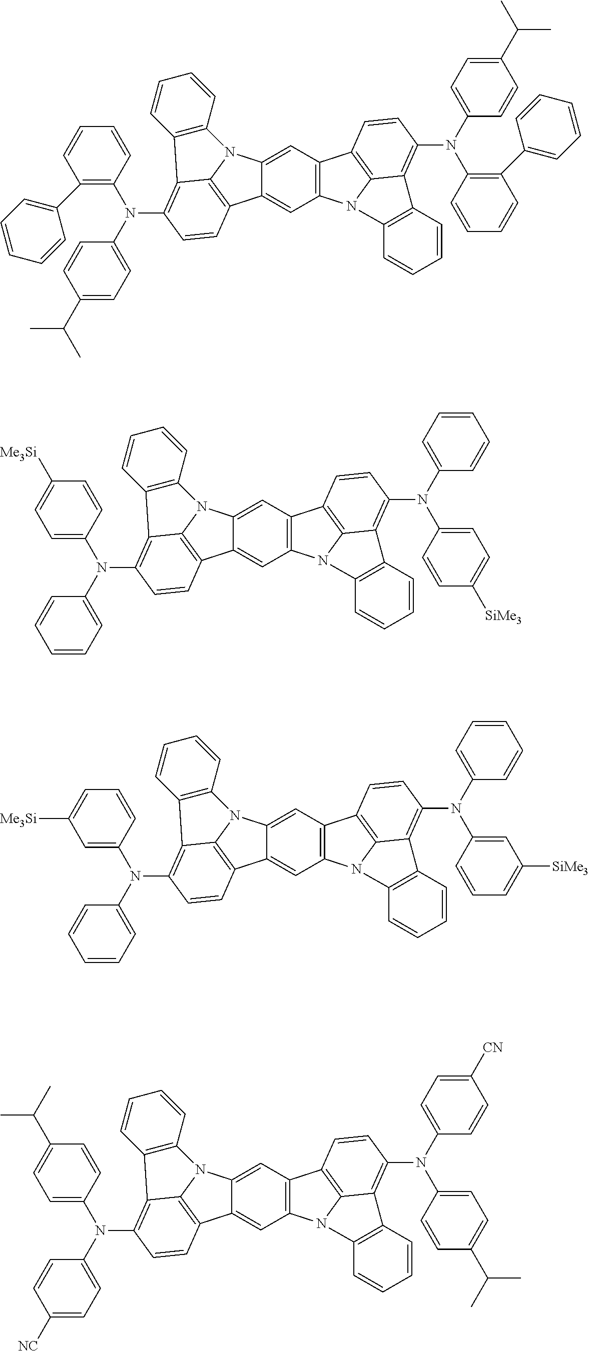 [SMILES: CC(C)c1ccc(N(c2ccccc2-c2ccccc2)c2ccc3c4cc5c(cc4n4c6ccccc6c2c34)c2ccc(N(c3ccc(C(C)C)cc3)c3ccccc3-c3ccccc3)c3c4ccccc4n5c23)cc1 ; C[Si](C)(C)c1ccc(N(c2ccccc2)c2ccc3c4cc5c(cc4n4c6ccccc6c2c34)c2ccc(N(c3ccccc3)c3ccc([Si](C)(C)C)cc3)c3c4ccccc4n5c23)cc1 ; C[Si](C)(C)c1cccc(N(c2ccccc2)c2ccc3c4cc5c(cc4n4c6ccccc6c2c34)c2ccc(N(c3ccccc3)c3cccc([Si](C)(C)C)c3)c3c4ccccc4n5c23)c1 ; CC(C)c1ccc(N(c2ccc(C#N)cc2)c2ccc3c4cc5c(cc4n4c6ccccc6c2c34)c2ccc(N(c3ccc(C#N)cc3)c3ccc(C(C)C)cc3)c3c4ccccc4n5c23)cc1]

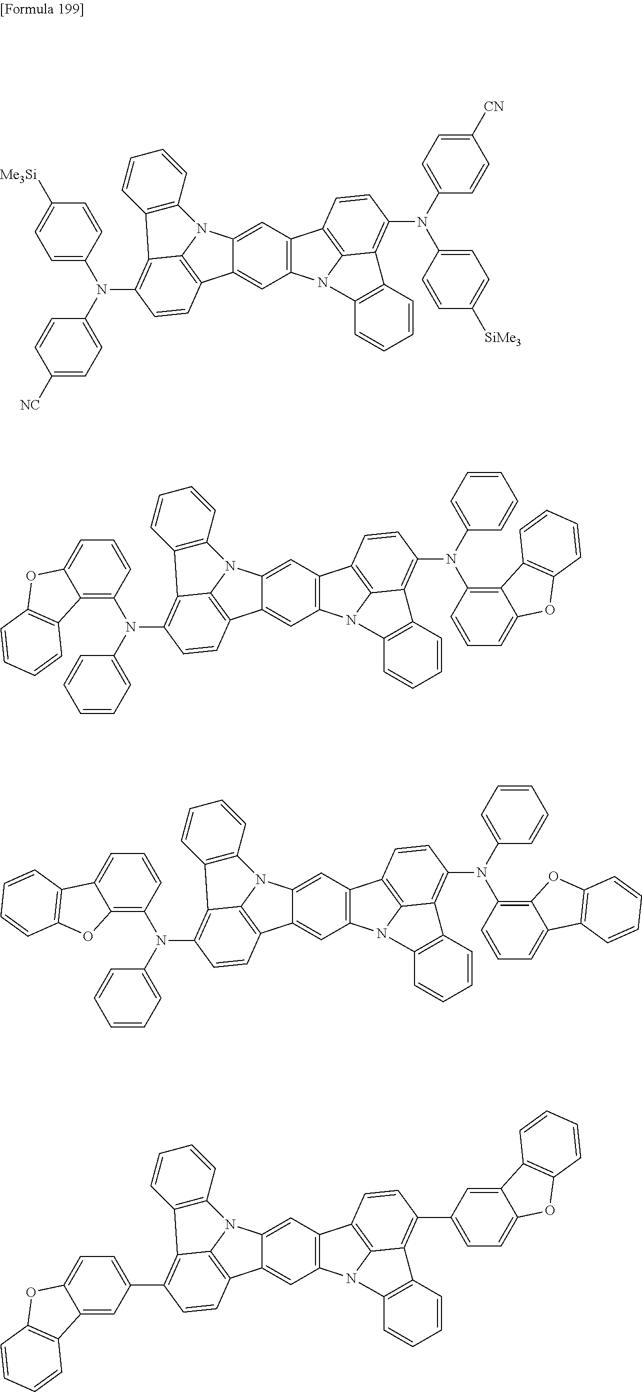 [SMILES: C[Si](C)(C)c1ccc(N(c2ccc(C#N)cc2)c2ccc3c4cc5c(cc4n4c6ccccc6c2c34)c2ccc(N(c3ccc(C#N)cc3)c3ccc([Si](C)(C)C)cc3)c3c4ccccc4n5c23)cc1 ; c1ccc(N(c2cccc3oc4ccccc4c23)c2ccc3c4cc5c(cc4n4c6ccccc6c2c34)c2ccc(N(c3ccccc3)c3cccc4oc6ccccc6c34)c3c4ccccc4n5c23)cc1 ; c1ccc(N(c2cccc3c2oc2ccccc23)c2ccc3c4cc5c(cc4n4c6ccccc6c2c34)c2ccc(N(c3ccccc3)c3cccc4c3oc3ccccc34)c3c4ccccc4n5c23)cc1 ; c1ccc2c(c1)oc1ccc(-c3ccc4c5cc6c(cc5n5c7ccccc7c3c45)c3ccc(-c4ccc5oc7ccccc7c5c4)c4c5ccccc5n6c34)cc12]

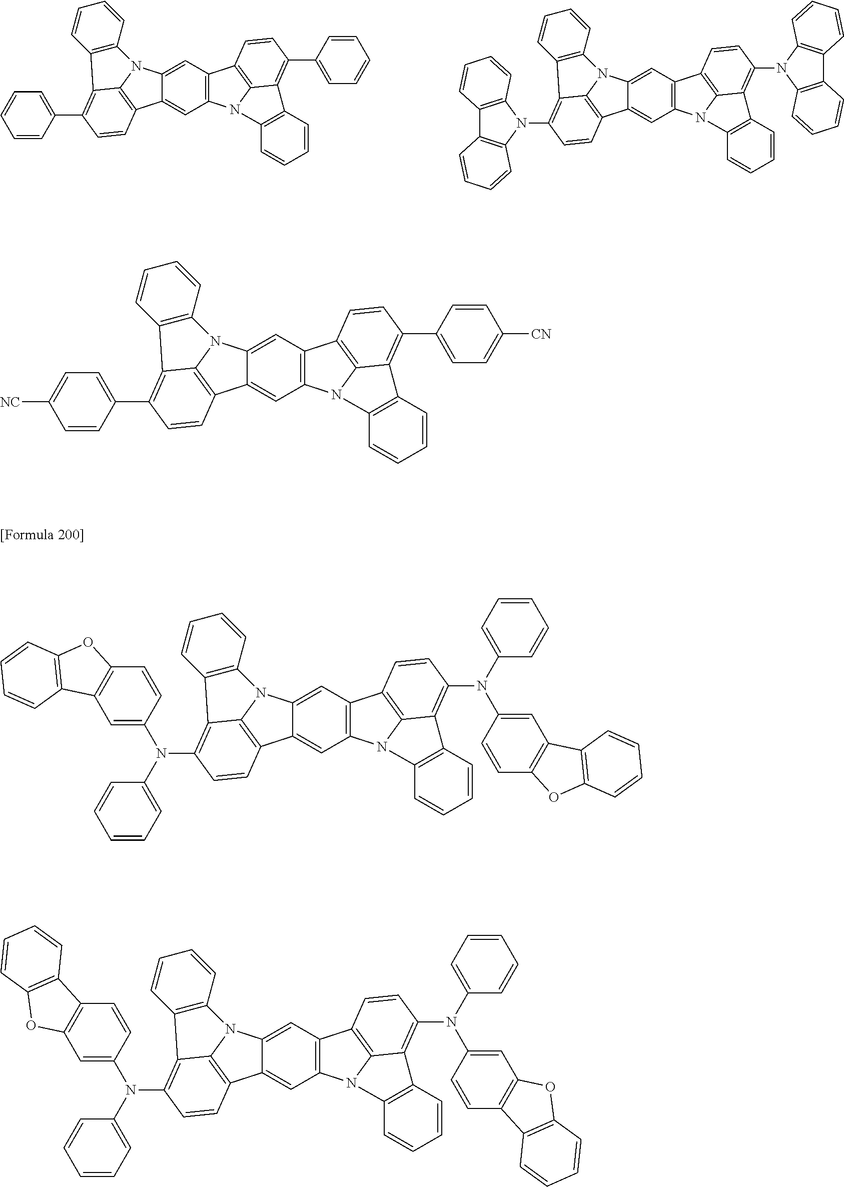 [SMILES: c1ccc(-c2ccc3c4cc5c(cc4n4c6ccccc6c2c34)c2ccc(-c3ccccc3)c3c4ccccc4n5c23)cc1 ; c1ccc2c(c1)c1ccccc1n2-c1ccc2c3cc4c(cc3n3c5ccccc5c1c23)c1ccc(-n2c3ccccc3c3ccccc32)c2c3ccccc3n4c12 ; N#Cc1ccc(-c2ccc3c4cc5c(cc4n4c6ccccc6c2c34)c2ccc(-c3ccc(C#N)cc3)c3c4ccccc4n5c23)cc1 ; c1ccc(N(c2ccc3oc4ccccc4c3c2)c2ccc3c4cc5c(cc4n4c6ccccc6c2c34)c2ccc(N(c3ccccc3)c3ccc4oc6ccccc6c4c3)c3c4ccccc4n5c23)cc1 ; c1ccc(N(c2ccc3c(c2)oc2ccccc23)c2ccc3c4cc5c(cc4n4c6ccccc6c2c34)c2ccc(N(c3ccccc3)c3ccc4c(c3)oc3ccccc34)c3c4ccccc4n5c23)cc1]

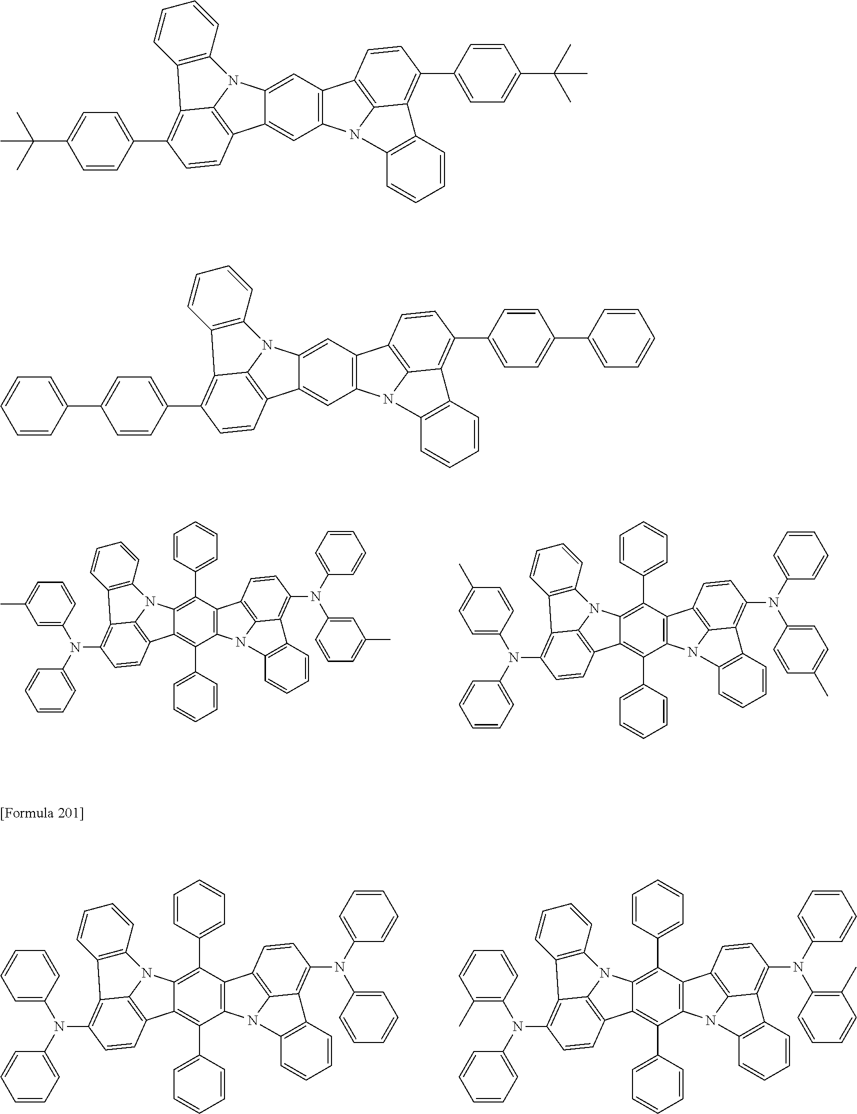

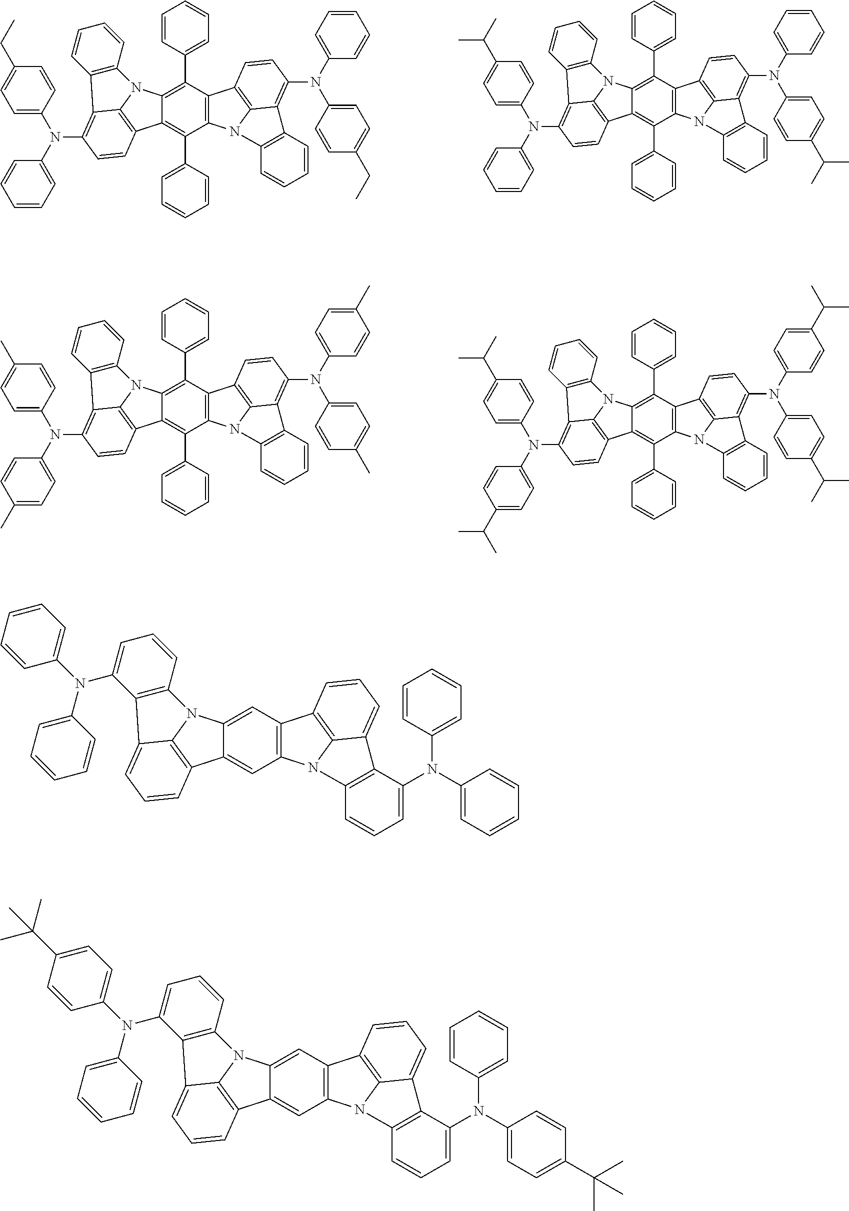

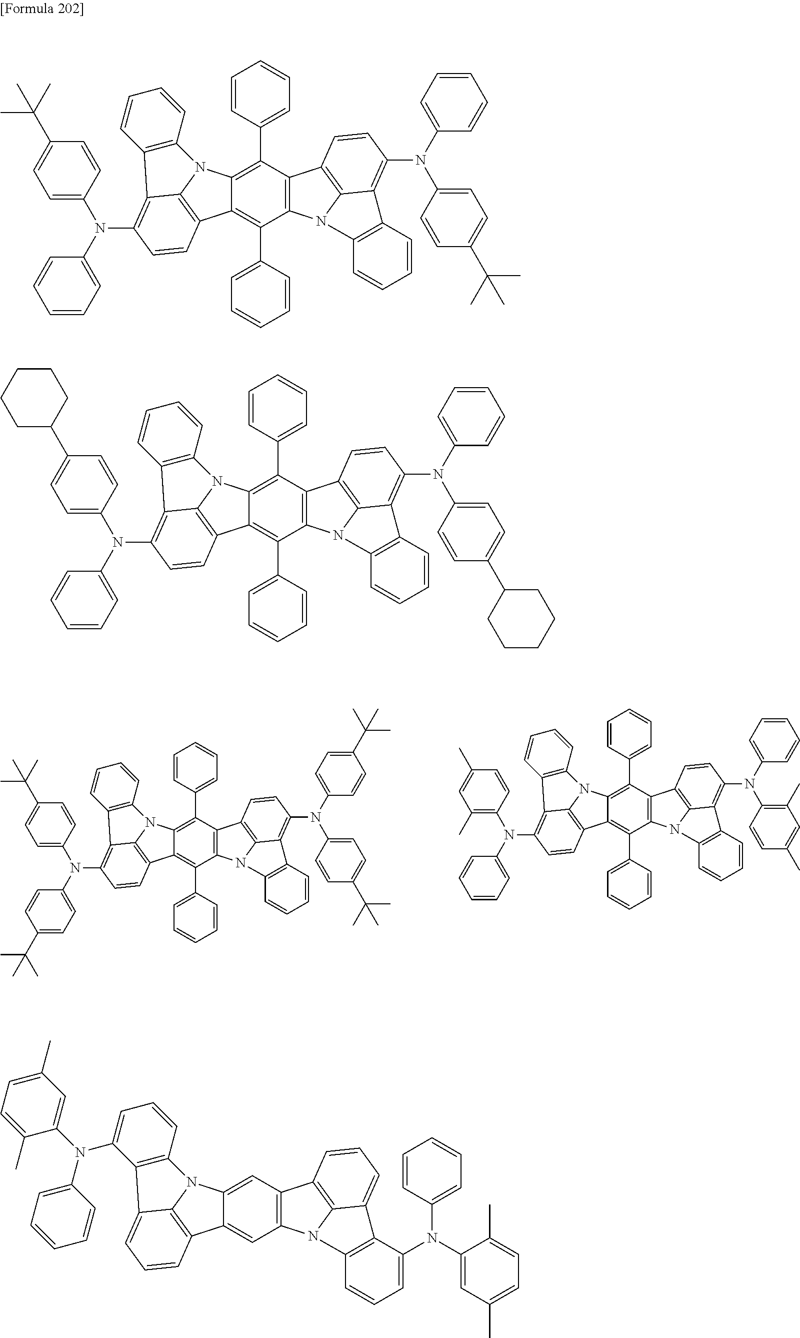

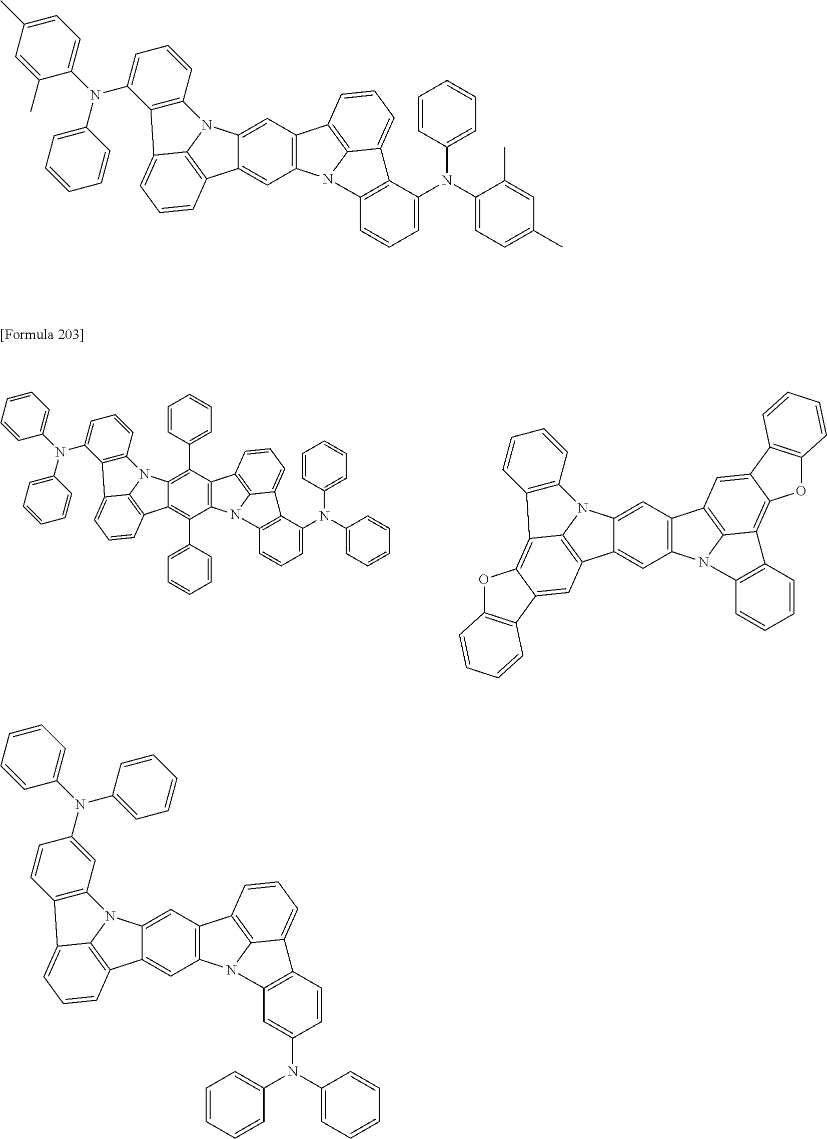

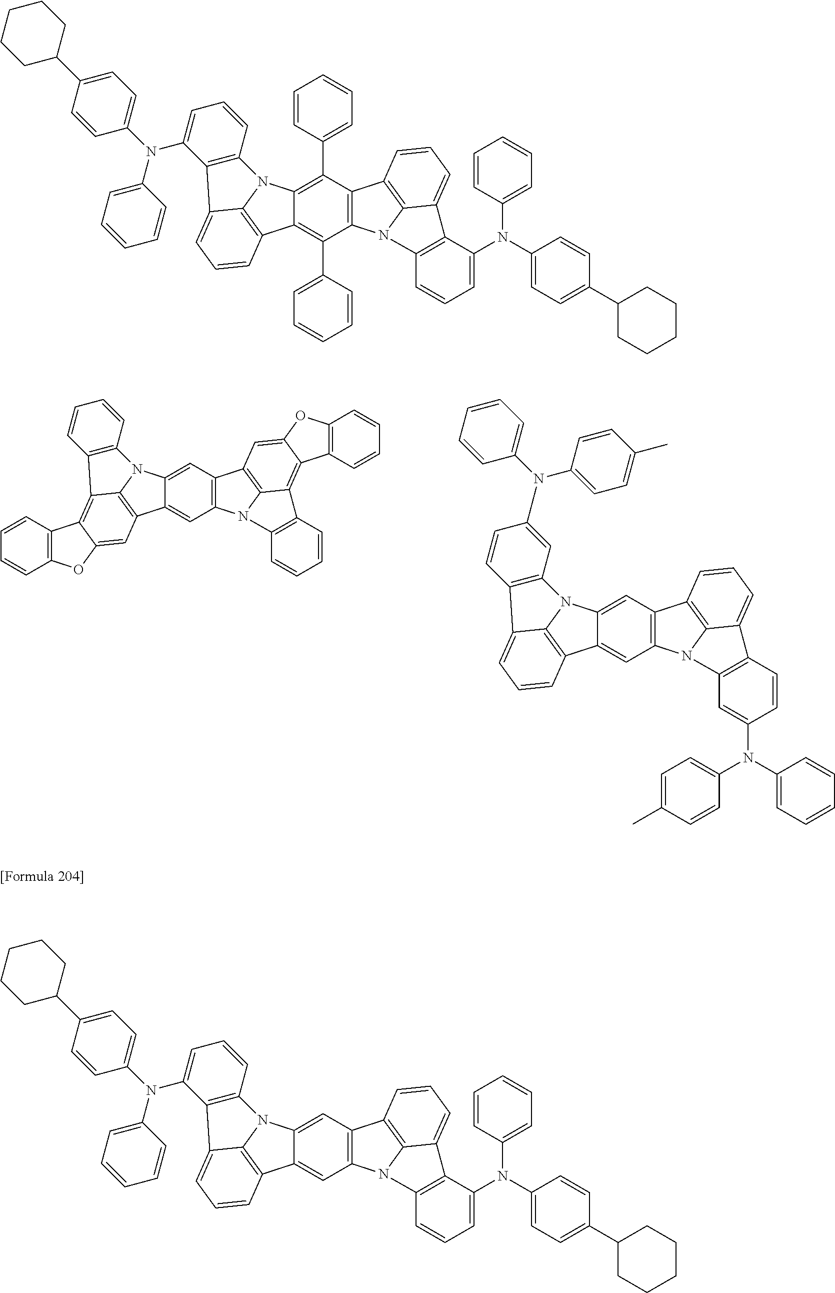

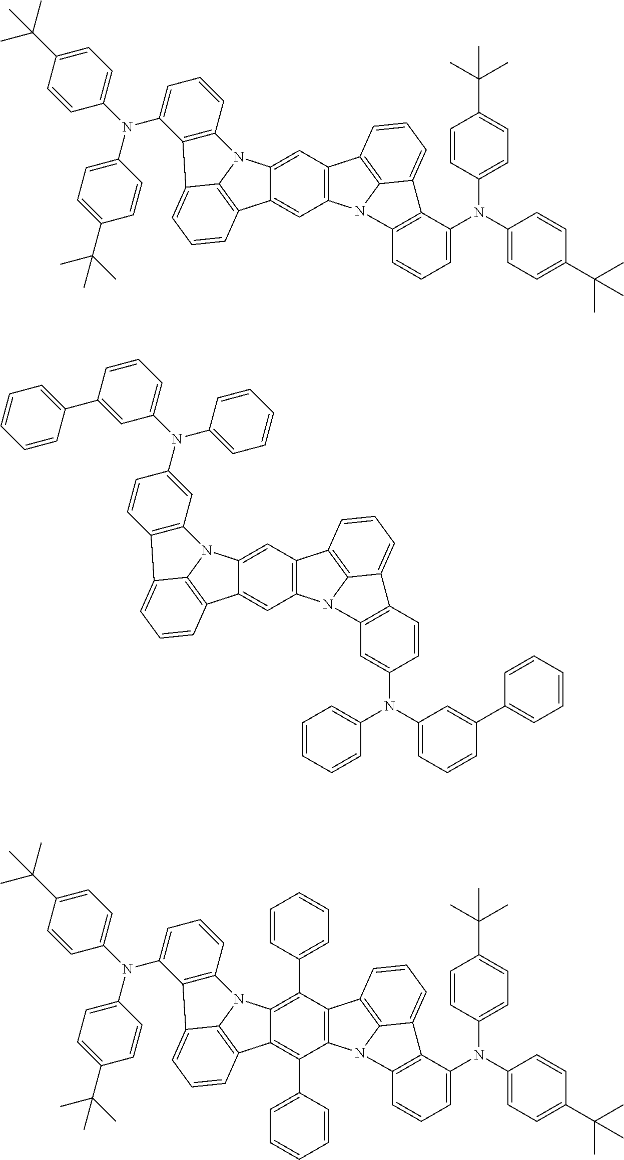

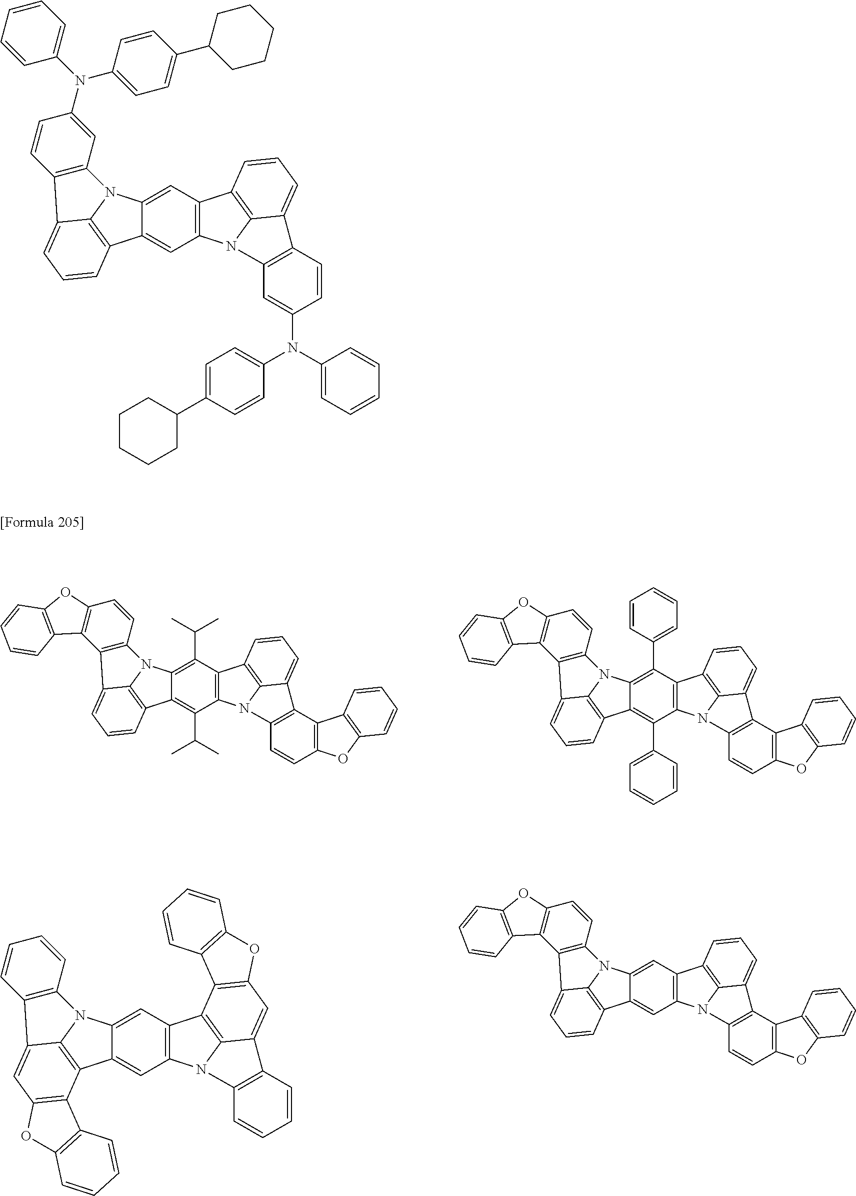

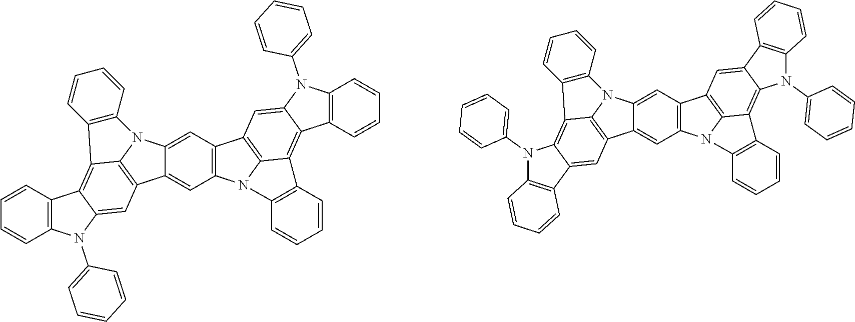 [SMILES: c1ccc(-n2c3ccccc3c3c4c5ccccc5n5c6cc7c8cc9c(c%10ccccc%10n9-c9ccccc9)c9c%10ccccc%10n(c7cc6c(cc32)c45)c89)cc1 ; c1ccc(-n2c3ccccc3c3cc4c5cc6c(cc5n5c7ccccc7c(c32)c45)c2cc3c4ccccc4n(-c4ccccc4)c3c3c4ccccc4n6c23)cc1]

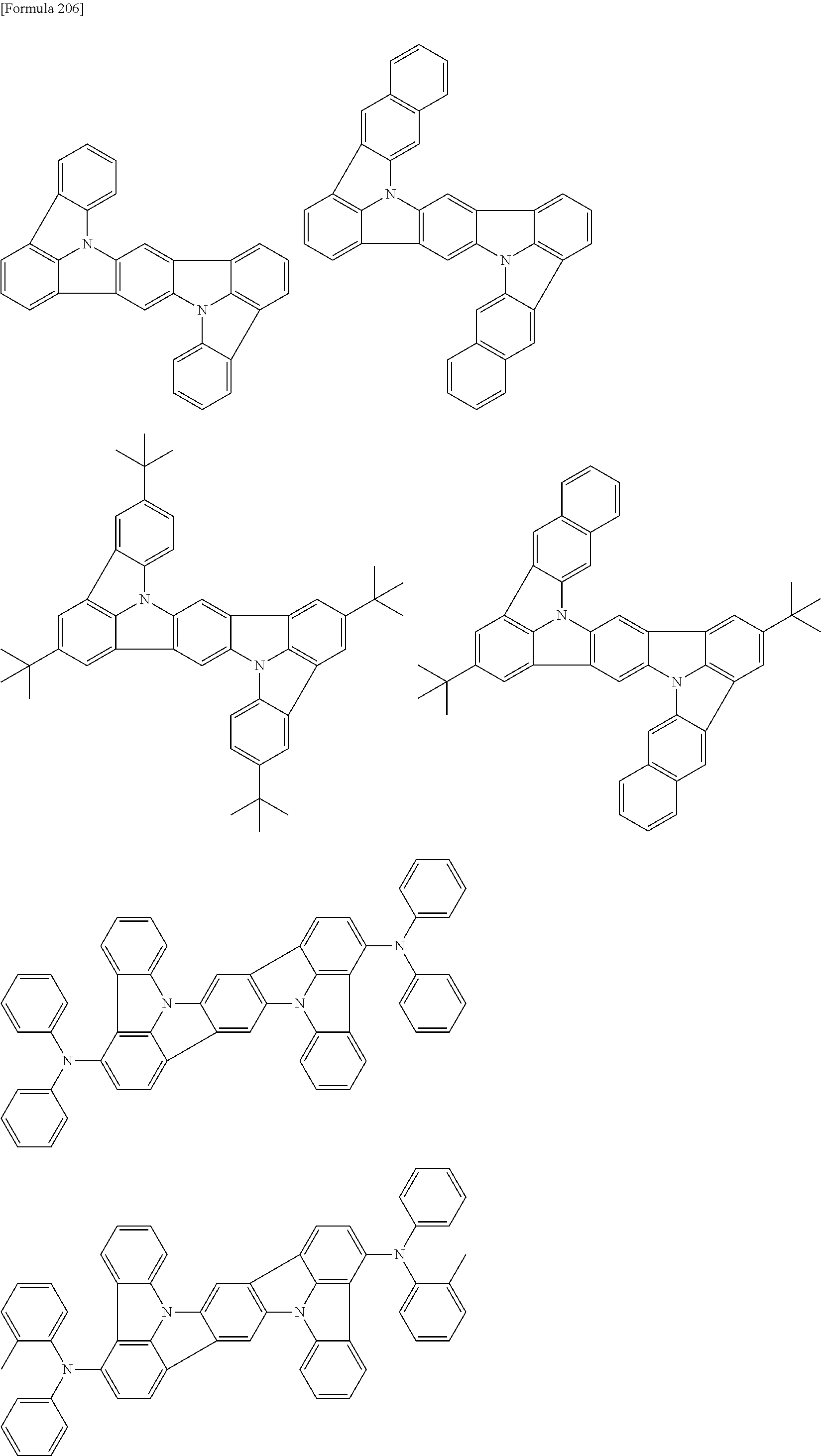

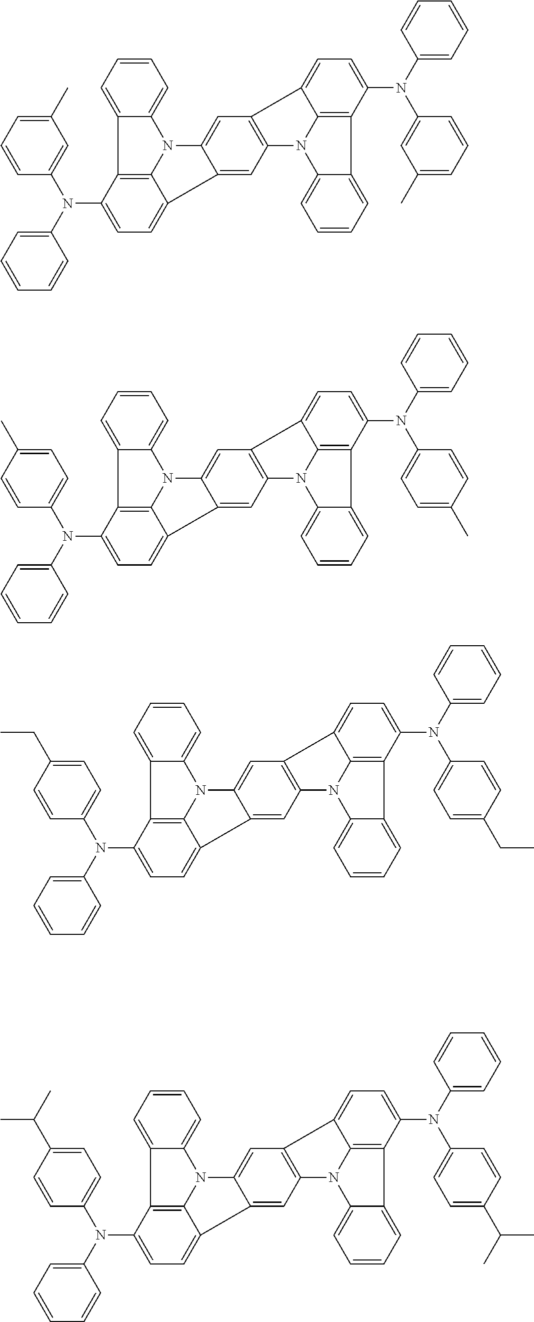

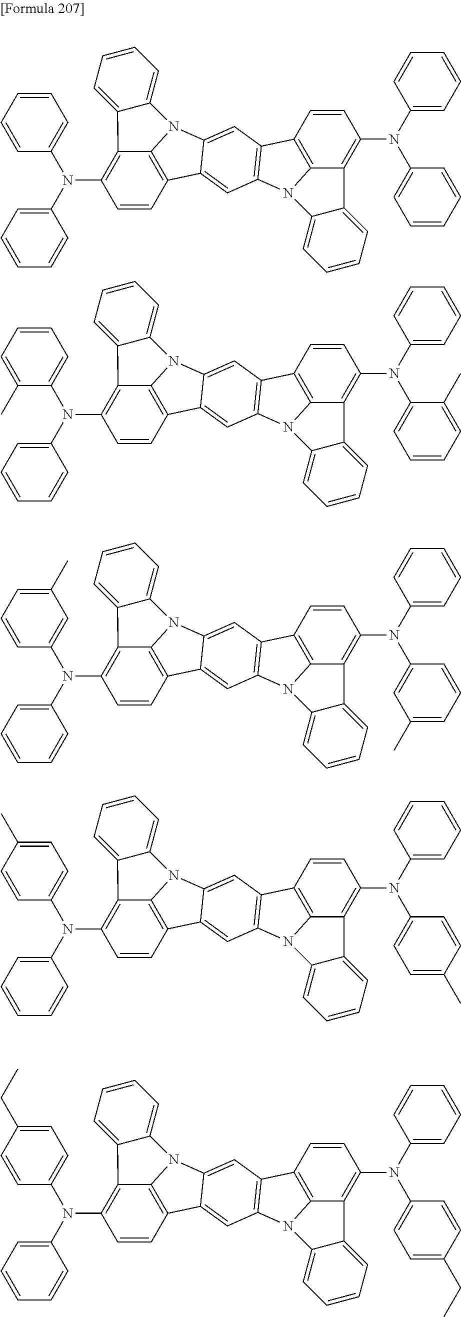 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1 ; Cc1ccc(N(c2ccccc2)c2ccc3c4cc5c(cc4n4c6ccccc6c2c34)c2ccc(N(c3ccccc3)c3ccc(C)cc3)c3c4ccccc4n5c23)cc1 ; CCc1ccc(N(c2ccccc2)c2ccc3c4cc5c(cc4n4c6ccccc6c2c34)c2ccc(N(c3ccccc3)c3ccc(CC)cc3)c3c4ccccc4n5c23)cc1]

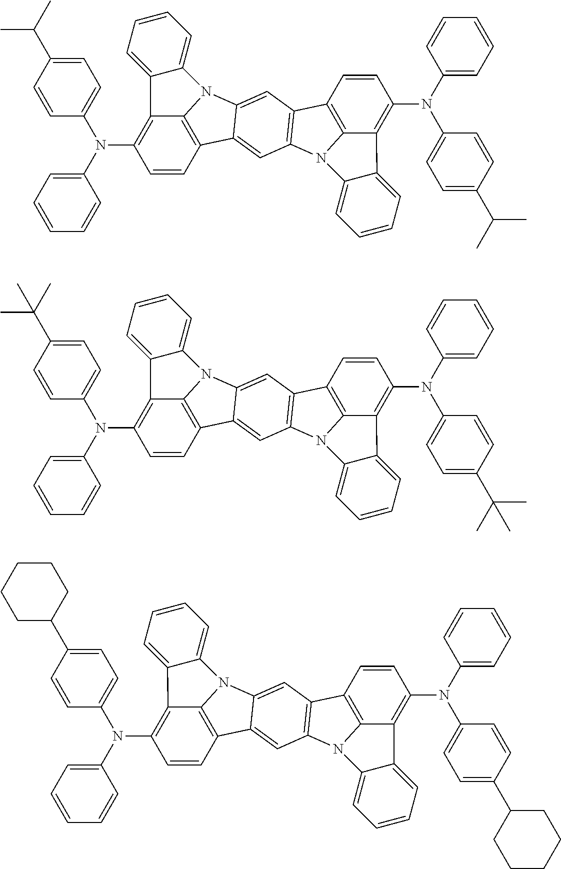

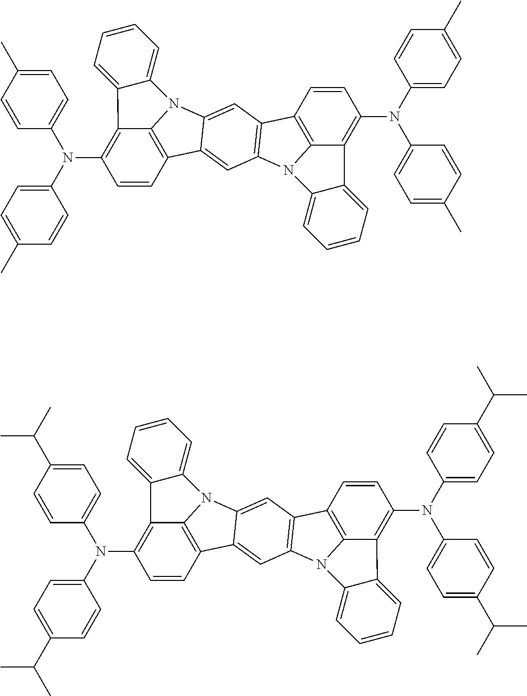

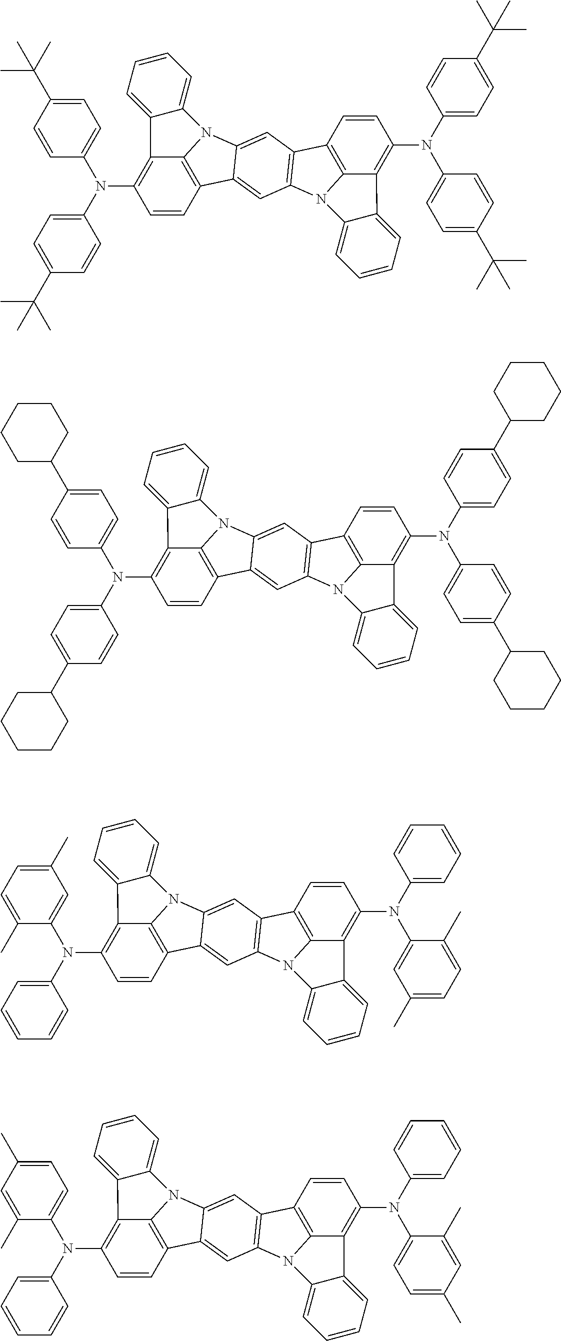 [SMILES: CC(C)(C)c1ccc(N(c2ccc(C(C)(C)C)cc2)c2ccc3c4cc5c(cc4n4c6ccccc6c2c34)c2ccc(N(c3ccc(C(C)(C)C)cc3)c3ccc(C(C)(C)C)cc3)c3c4ccccc4n5c23)cc1 ; c1ccc2c(c1)c1c(N(c3ccc(C4CCCCC4)cc3)c3ccc(C4CCCCC4)cc3)ccc3c4cc5c(cc4n2c31)c1ccc(N(c2ccc(C3CCCCC3)cc2)c2ccc(C3CCCCC3)cc2)c2c3ccccc3n5c12 ; Cc1ccc(C)c(N(c2ccccc2)c2ccc3c4cc5c(cc4n4c6ccccc6c2c34)c2ccc(N(c3ccccc3)c3cc(C)ccc3C)c3c4ccccc4n5c23)c1 ; Cc1ccc(N(c2ccccc2)c2ccc3c4cc5c(cc4n4c6ccccc6c2c34)c2ccc(N(c3ccccc3)c3ccc(C)cc3C)c3c4ccccc4n5c23)c(C)c1]

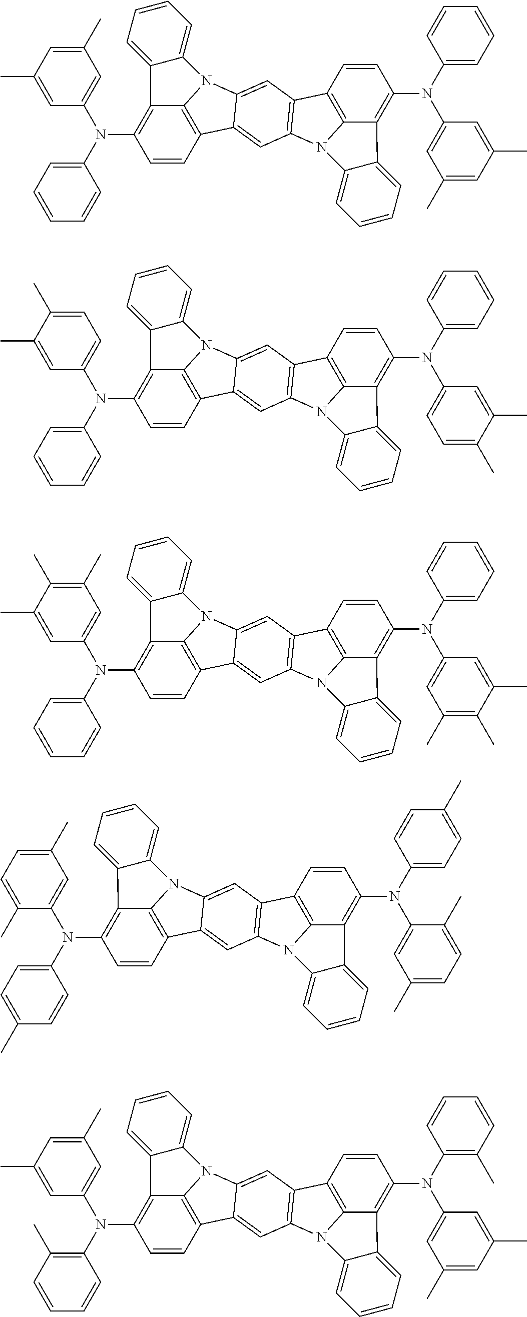

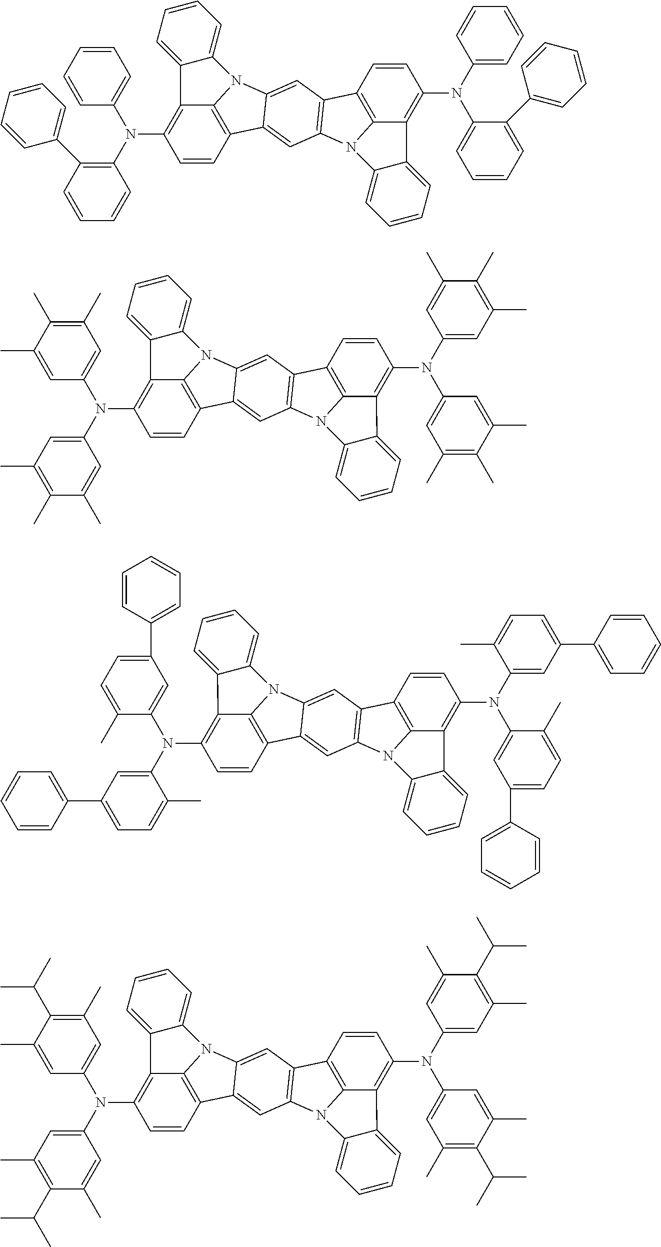

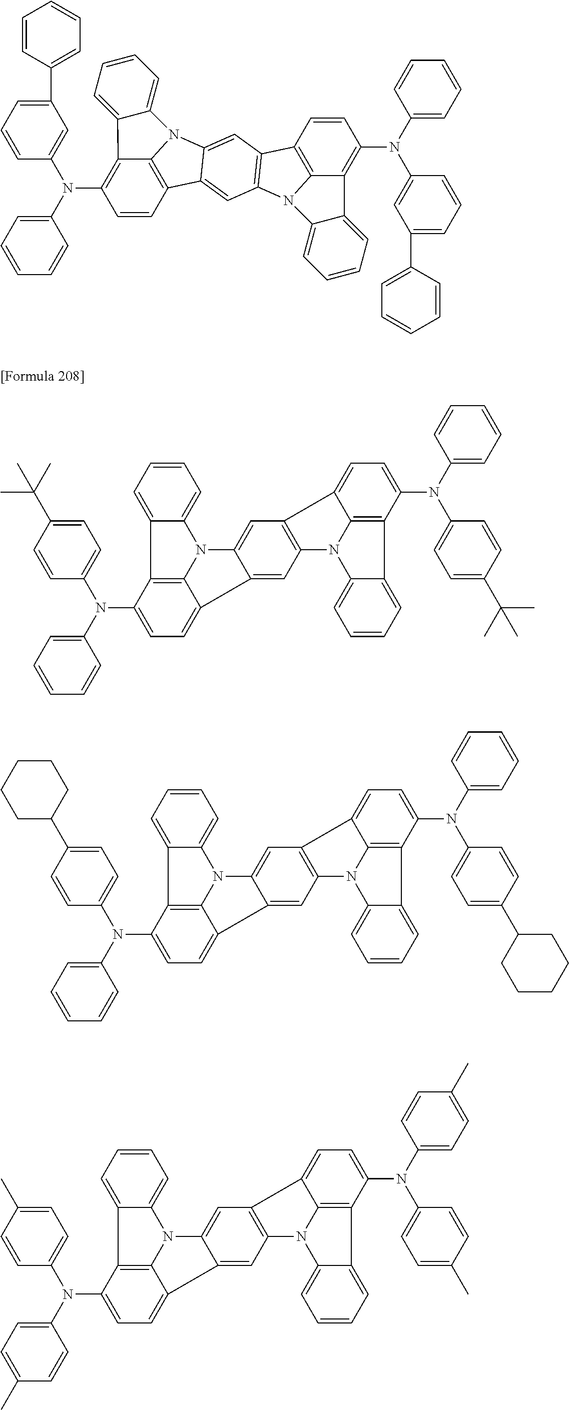 [SMILES: c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1 ; CC(C)(C)c1ccc(N(c2ccccc2)c2ccc3c4cc5c(cc4n4c6ccccc6c2c34)c2ccc(N(c3ccccc3)c3ccc(C(C)(C)C)cc3)c3c4ccccc4n5c23)cc1 ; c1ccc(N(c2ccc(C3CCCCC3)cc2)c2ccc3c4cc5c(cc4n4c6ccccc6c2c34)c2ccc(N(c3ccccc3)c3ccc(C4CCCCC4)cc3)c3c4ccccc4n5c23)cc1 ; Cc1ccc(N(c2ccc(C)cc2)c2ccc3c4cc5c(cc4n4c6ccccc6c2c34)c2ccc(N(c3ccc(C)cc3)c3ccc(C)cc3)c3c4ccccc4n5c23)cc1]

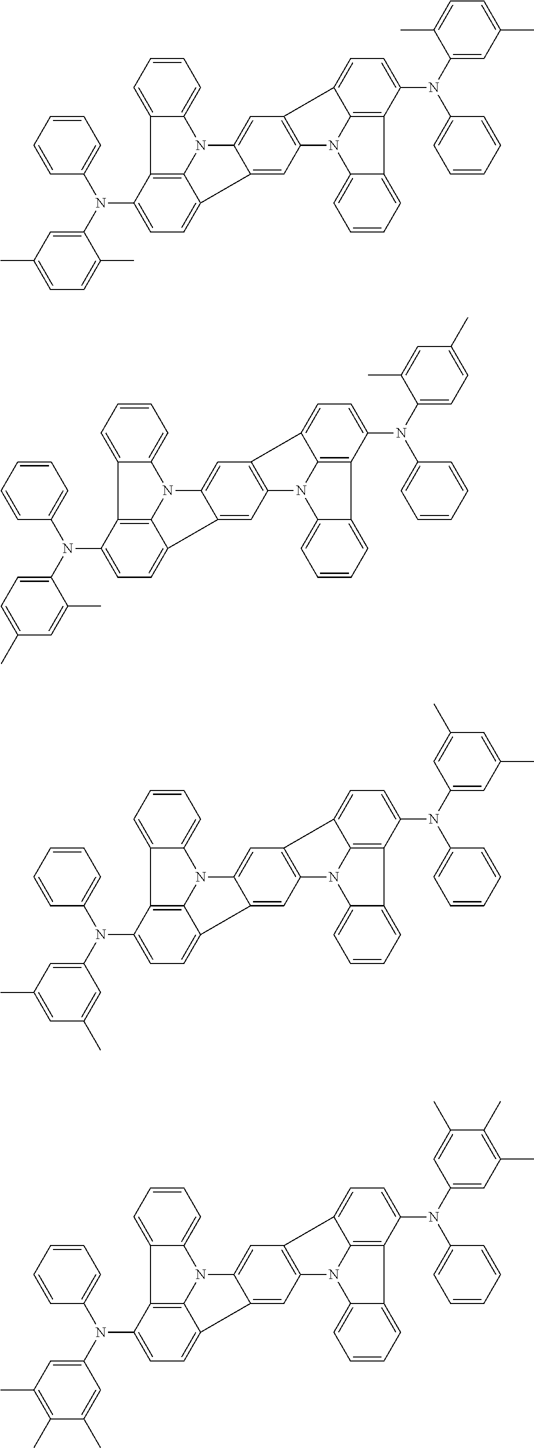

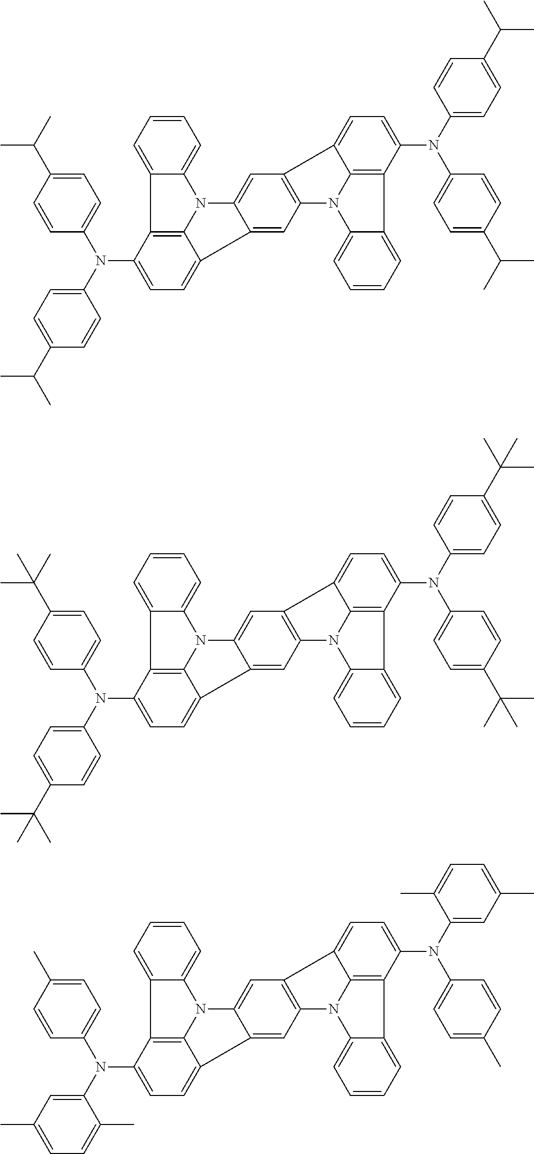

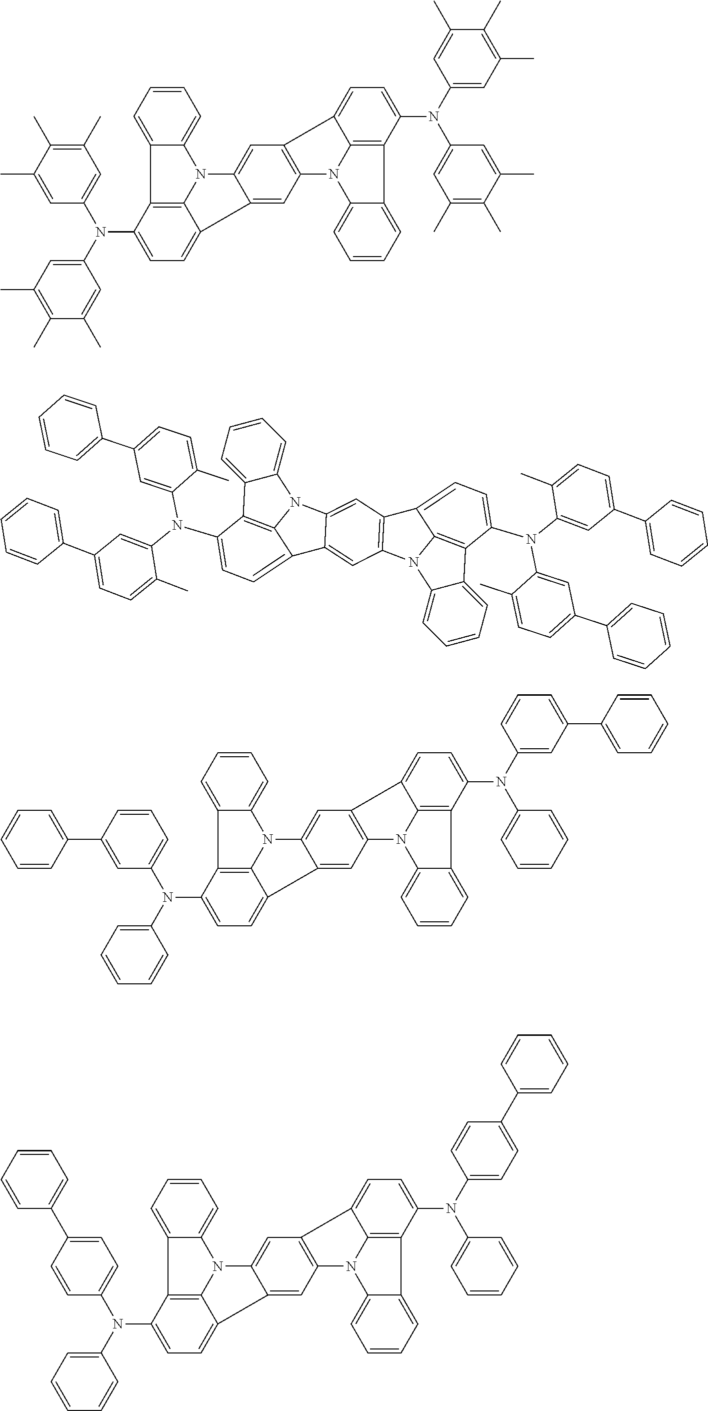

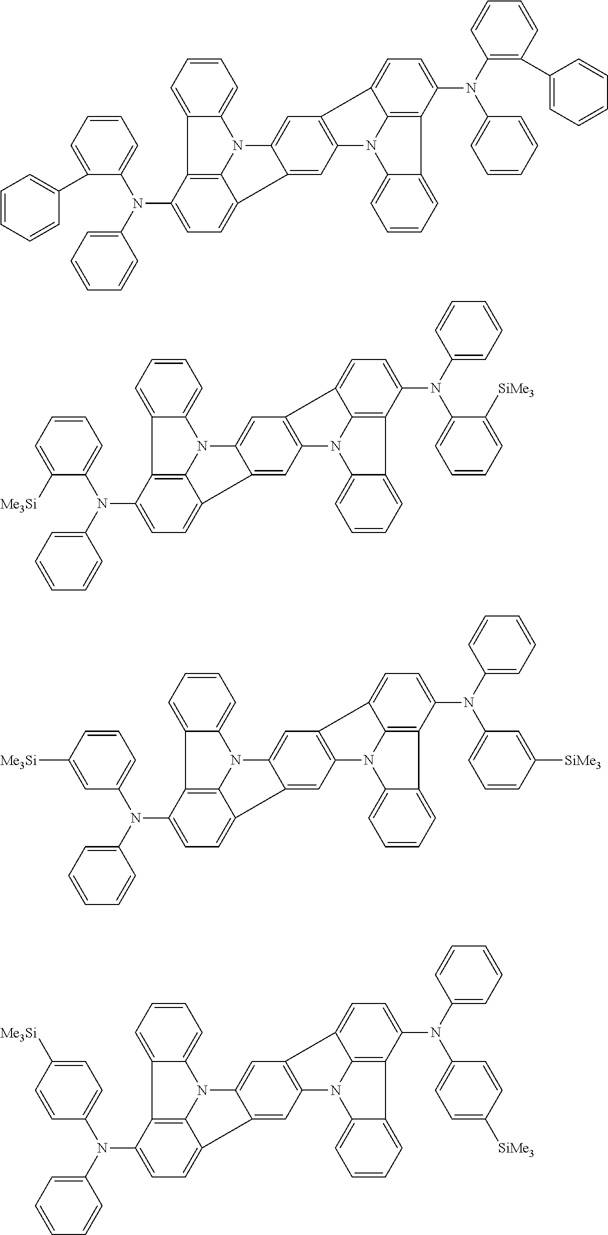 [SMILES: c1ccc(-c2ccccc2N(c2ccccc2)c2ccc3c4cc5c(cc4n4c6ccccc6c2c34)c2ccc(N(c3ccccc3)c3ccccc3-c3ccccc3)c3c4ccccc4n5c23)cc1 ; C[Si](C)(C)c1ccccc1N(c1ccccc1)c1ccc2c3cc4c(cc3n3c5ccccc5c1c23)c1ccc(N(c2ccccc2)c2ccccc2[Si](C)(C)C)c2c3ccccc3n4c12 ; C[Si](C)(C)c1cccc(N(c2ccccc2)c2ccc3c4cc5c(cc4n4c6ccccc6c2c34)c2ccc(N(c3ccccc3)c3cccc([Si](C)(C)C)c3)c3c4ccccc4n5c23)c1 ; C[Si](C)(C)c1ccc(N(c2ccccc2)c2ccc3c4cc5c(cc4n4c6ccccc6c2c34)c2ccc(N(c3ccccc3)c3ccc([Si](C)(C)C)cc3)c3c4ccccc4n5c23)cc1]

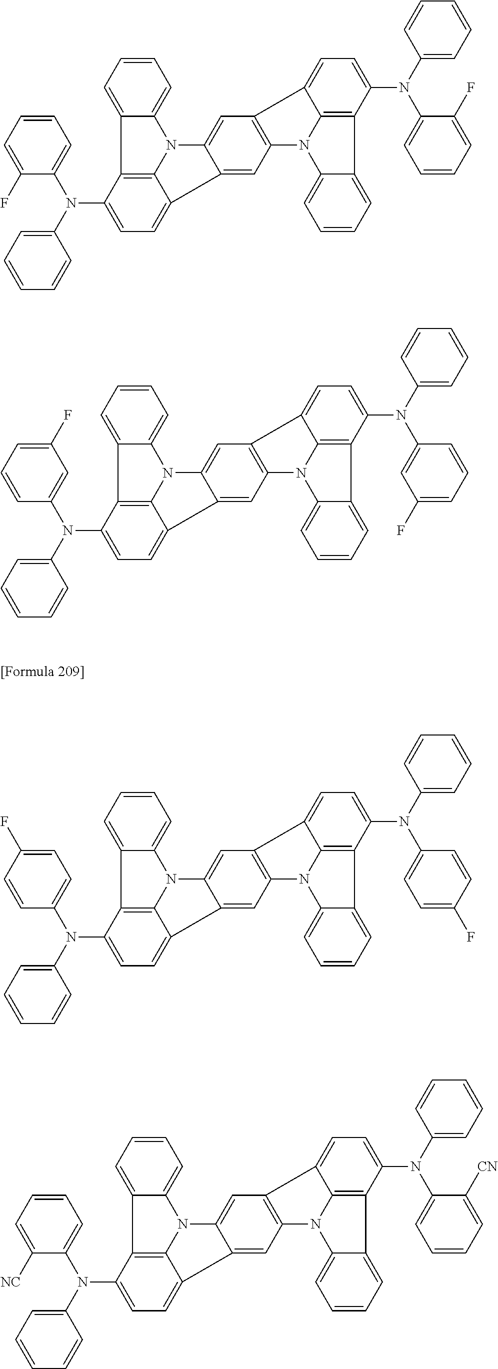 [SMILES: Fc1ccccc1N(c1ccccc1)c1ccc2c3cc4c(cc3n3c5ccccc5c1c23)c1ccc(N(c2ccccc2)c2ccccc2F)c2c3ccccc3n4c12 ; Fc1cccc(N(c2ccccc2)c2ccc3c4cc5c(cc4n4c6ccccc6c2c34)c2ccc(N(c3ccccc3)c3cccc(F)c3)c3c4ccccc4n5c23)c1 ; Fc1ccc(N(c2ccccc2)c2ccc3c4cc5c(cc4n4c6ccccc6c2c34)c2ccc(N(c3ccccc3)c3ccc(F)cc3)c3c4ccccc4n5c23)cc1 ; N#Cc1ccccc1N(c1ccccc1)c1ccc2c3cc4c(cc3n3c5ccccc5c1c23)c1ccc(N(c2ccccc2)c2ccccc2C#N)c2c3ccccc3n4c12]

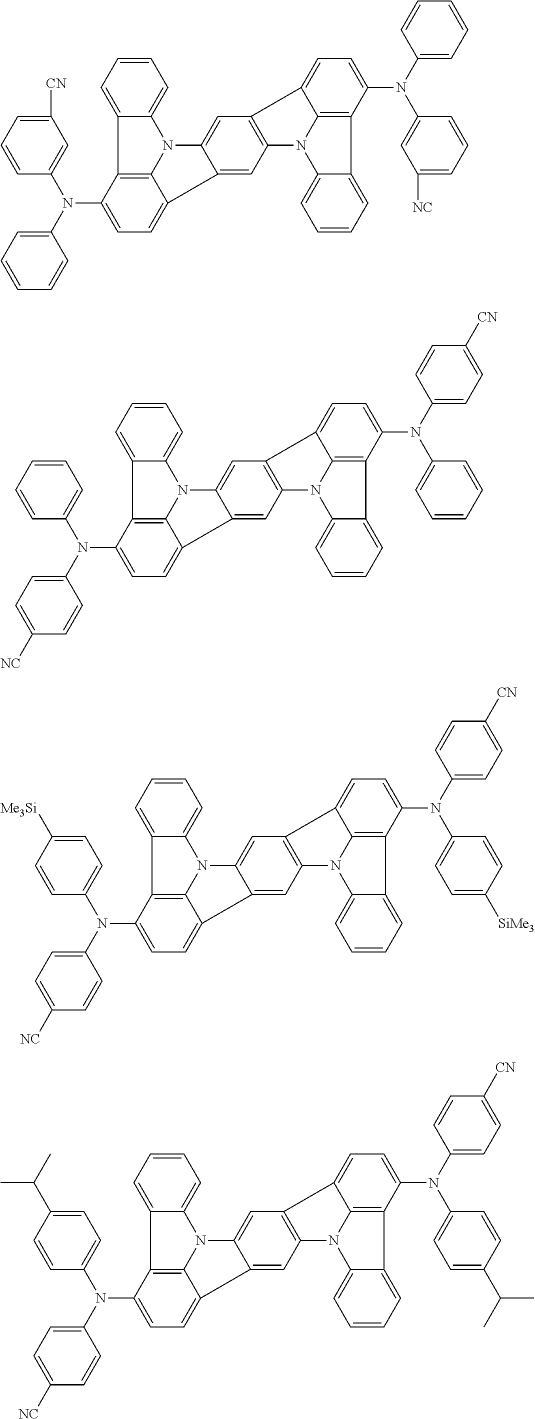

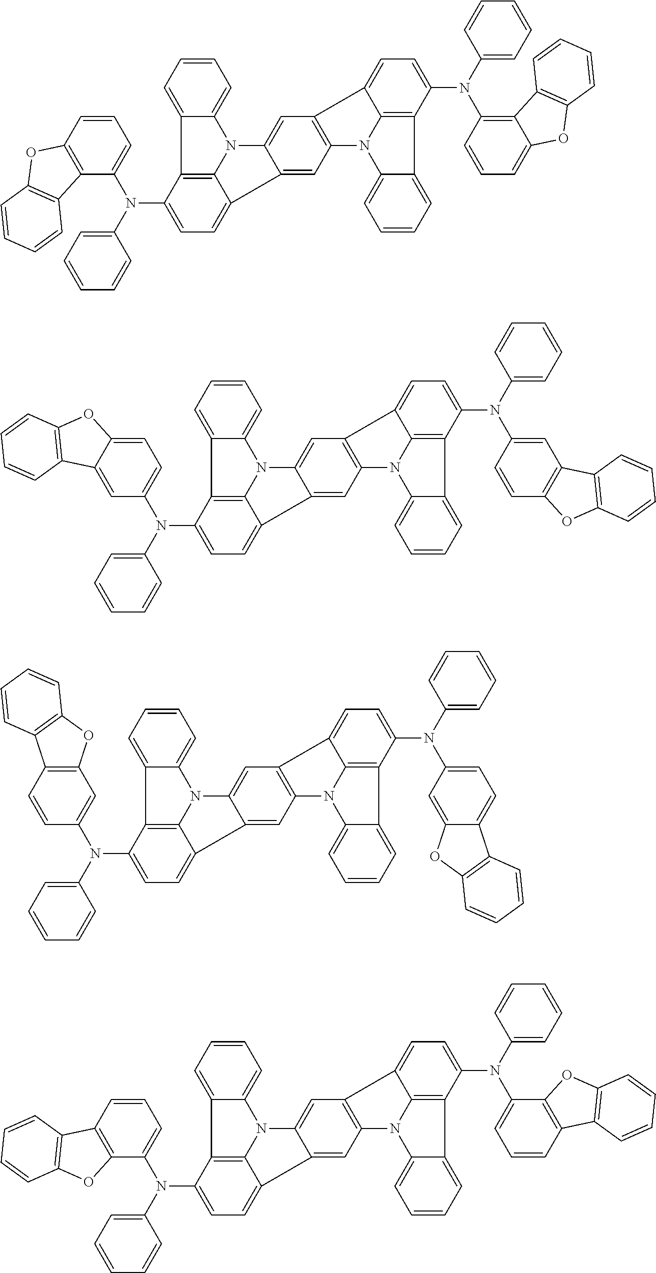 [SMILES: c1ccc(N(c2cccc3oc4ccccc4c23)c2ccc3c4cc5c(cc4n4c6ccccc6c2c34)c2ccc(N(c3ccccc3)c3cccc4oc6ccccc6c34)c3c4ccccc4n5c23)cc1 ; c1ccc(N(c2ccc3oc4ccccc4c3c2)c2ccc3c4cc5c(cc4n4c6ccccc6c2c34)c2ccc(N(c3ccccc3)c3ccc4oc6ccccc6c4c3)c3c4ccccc4n5c23)cc1 ; c1ccc(N(c2ccc3c(c2)oc2ccccc23)c2ccc3c4cc5c(cc4n4c6ccccc6c2c34)c2ccc(N(c3ccccc3)c3ccc4c(c3)oc3ccccc34)c3c4ccccc4n5c23)cc1 ; c1ccc(N(c2cccc3c2oc2ccccc23)c2ccc3c4cc5c(cc4n4c6ccccc6c2c34)c2ccc(N(c3ccccc3)c3cccc4c3oc3ccccc34)c3c4ccccc4n5c23)cc1]

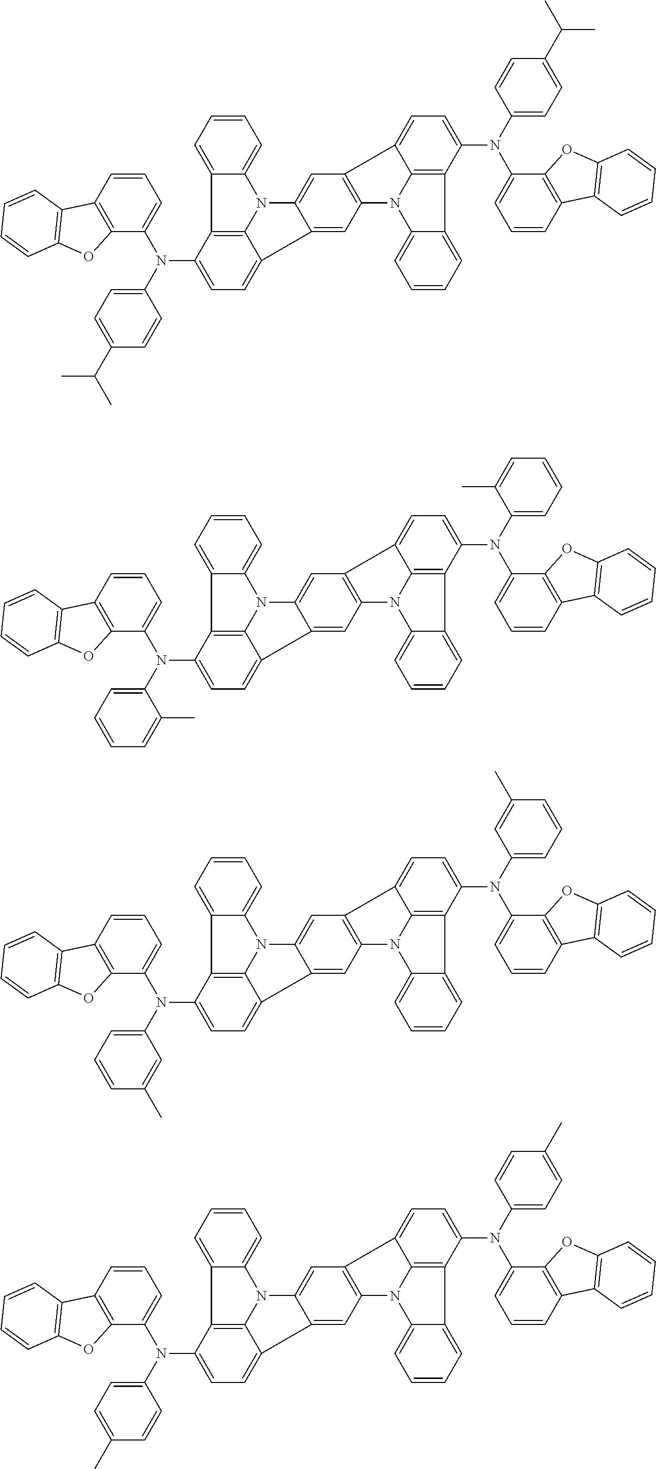 [SMILES: CC(C)c1ccc(N(c2cccc3c2oc2ccccc23)c2ccc3c4cc5c(cc4n4c6ccccc6c2c34)c2ccc(N(c3ccc(C(C)C)cc3)c3cccc4c3oc3ccccc34)c3c4ccccc4n5c23)cc1 ; Cc1ccccc1N(c1cccc2c1oc1ccccc12)c1ccc2c3cc4c(cc3n3c5ccccc5c1c23)c1ccc(N(c2ccccc2C)c2cccc3c2oc2ccccc23)c2c3ccccc3n4c12 ; Cc1cccc(N(c2cccc3c2oc2ccccc23)c2ccc3c4cc5c(cc4n4c6ccccc6c2c34)c2ccc(N(c3cccc(C)c3)c3cccc4c3oc3ccccc34)c3c4ccccc4n5c23)c1 ; Cc1ccc(N(c2cccc3c2oc2ccccc23)c2ccc3c4cc5c(cc4n4c6ccccc6c2c34)c2ccc(N(c3ccc(C)cc3)c3cccc4c3oc3ccccc34)c3c4ccccc4n5c23)cc1]

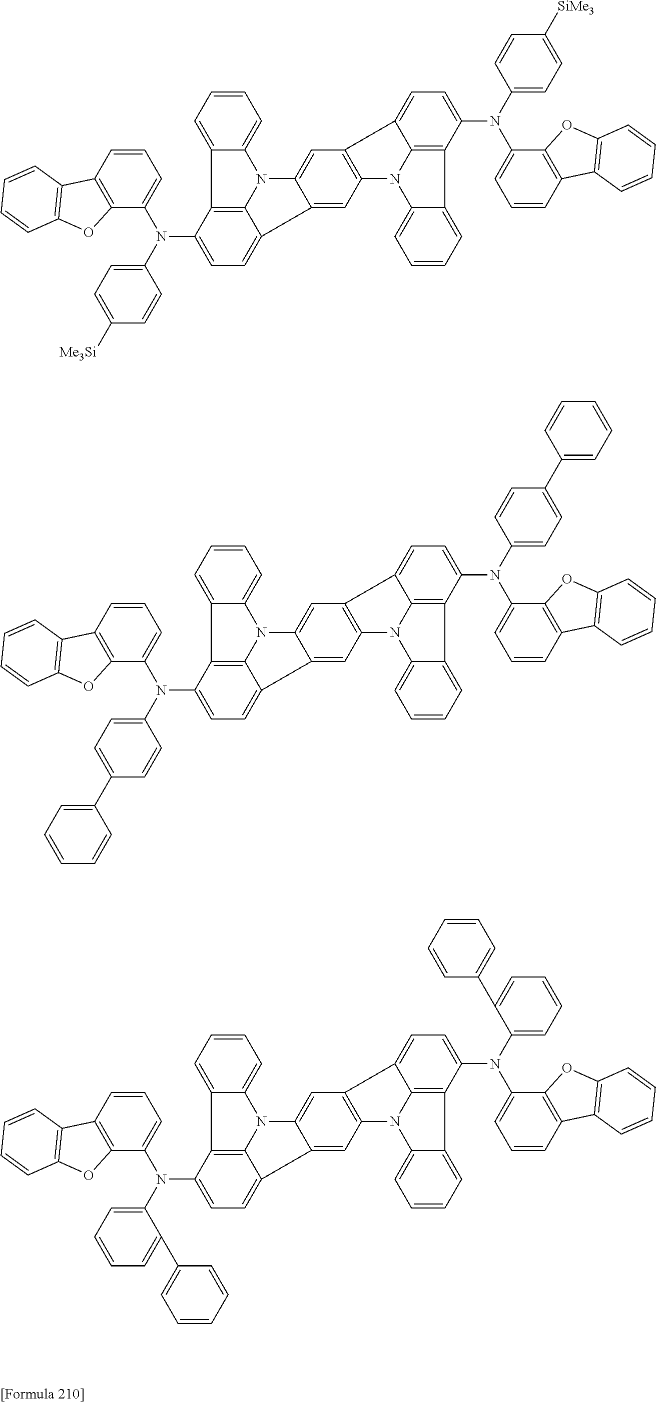 [SMILES: C[Si](C)(C)c1ccc(N(c2cccc3c2oc2ccccc23)c2ccc3c4cc5c(cc4n4c6ccccc6c2c34)c2ccc(N(c3ccc([Si](C)(C)C)cc3)c3cccc4c3oc3ccccc34)c3c4ccccc4n5c23)cc1 ; c1ccc(-c2ccc(N(c3cccc4c3oc3ccccc34)c3ccc4c5cc6c(cc5n5c7ccccc7c3c45)c3ccc(N(c4ccc(-c5ccccc5)cc4)c4cccc5c4oc4ccccc45)c4c5ccccc5n6c34)cc2)cc1 ; c1ccc(-c2ccccc2N(c2cccc3c2oc2ccccc23)c2ccc3c4cc5c(cc4n4c6ccccc6c2c34)c2ccc(N(c3ccccc3-c3ccccc3)c3cccc4c3oc3ccccc34)c3c4ccccc4n5c23)cc1]

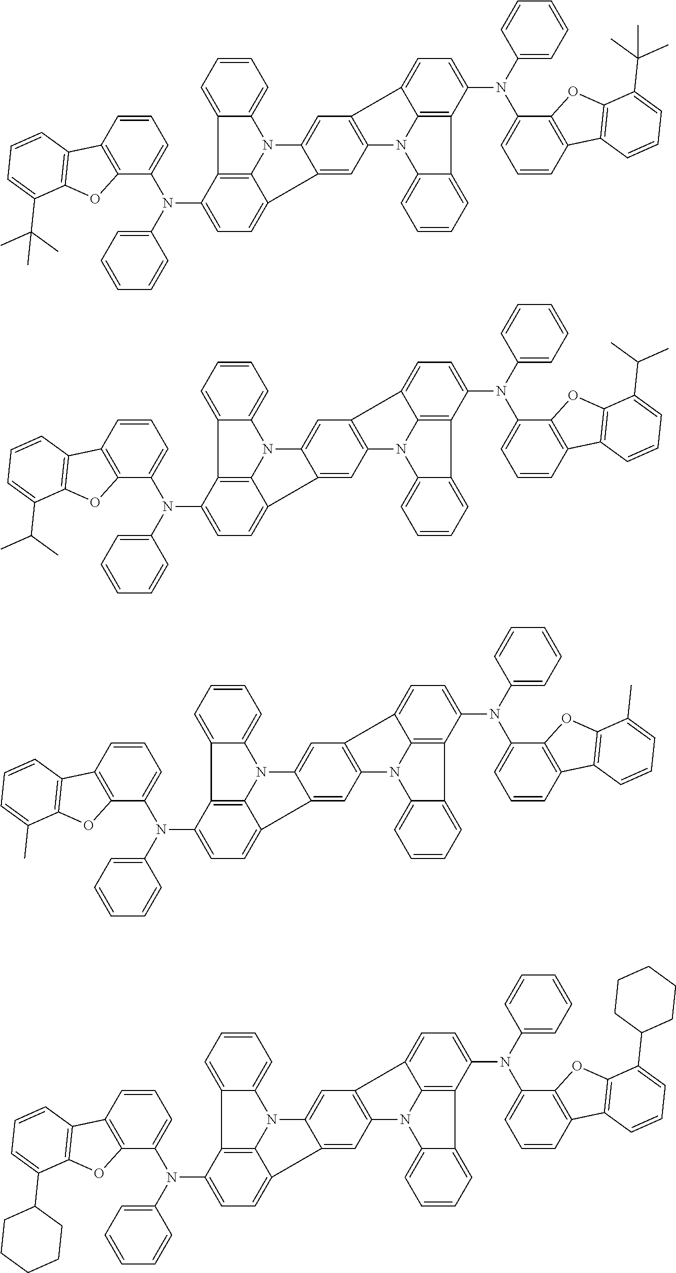

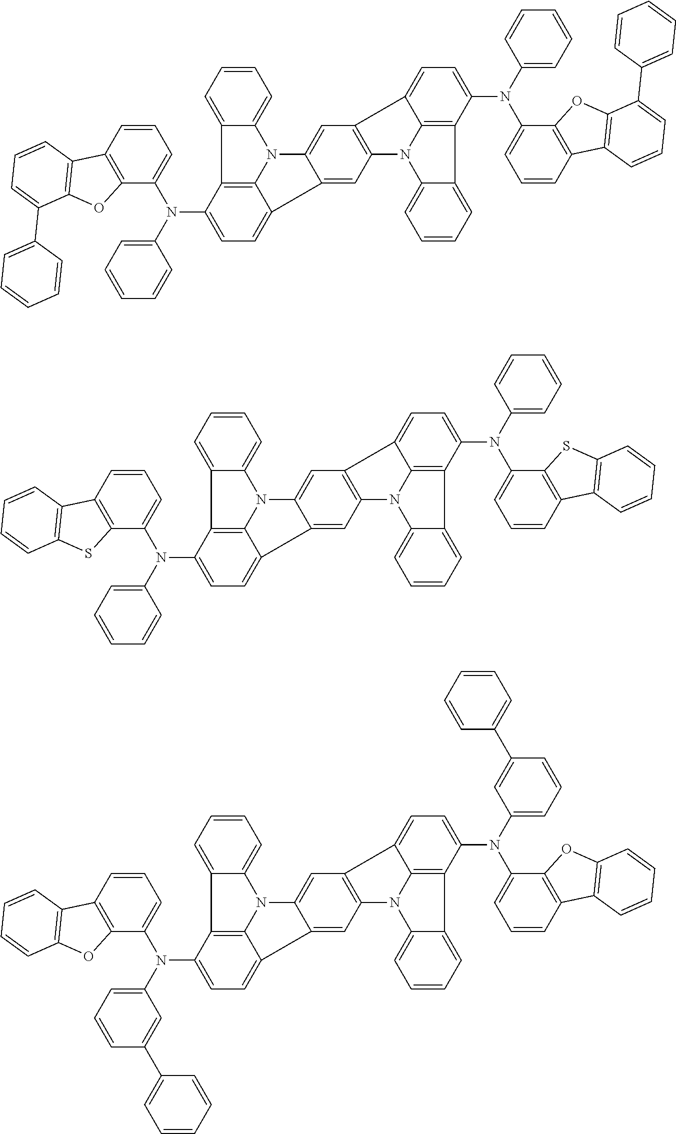 [SMILES: c1ccc(-c2cccc3c2oc2c(N(c4ccccc4)c4ccc5c6cc7c(cc6n6c8ccccc8c4c56)c4ccc(N(c5ccccc5)c5cccc6c5oc5c(-c8ccccc8)cccc56)c5c6ccccc6n7c45)cccc23)cc1 ; c1ccc(N(c2cccc3c2sc2ccccc23)c2ccc3c4cc5c(cc4n4c6ccccc6c2c34)c2ccc(N(c3ccccc3)c3cccc4c3sc3ccccc34)c3c4ccccc4n5c23)cc1 ; c1ccc(-c2cccc(N(c3cccc4c3oc3ccccc34)c3ccc4c5cc6c(cc5n5c7ccccc7c3c45)c3ccc(N(c4cccc(-c5ccccc5)c4)c4cccc5c4oc4ccccc45)c4c5ccccc5n6c34)c2)cc1]

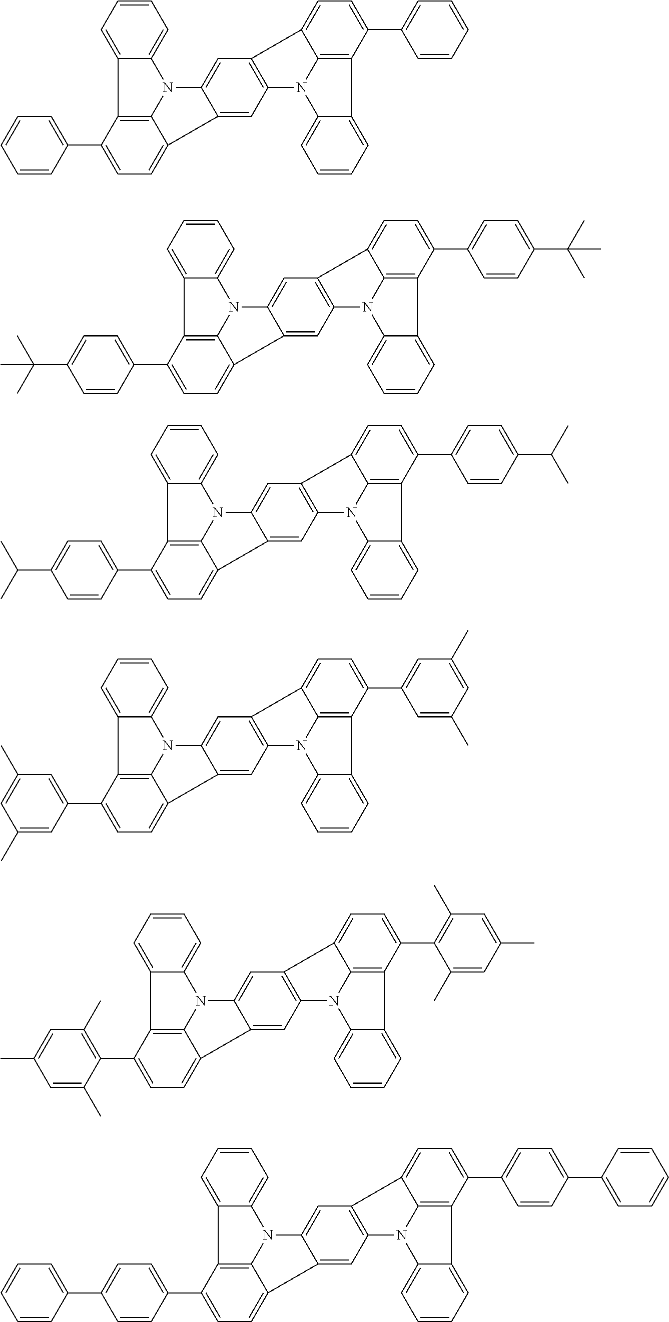

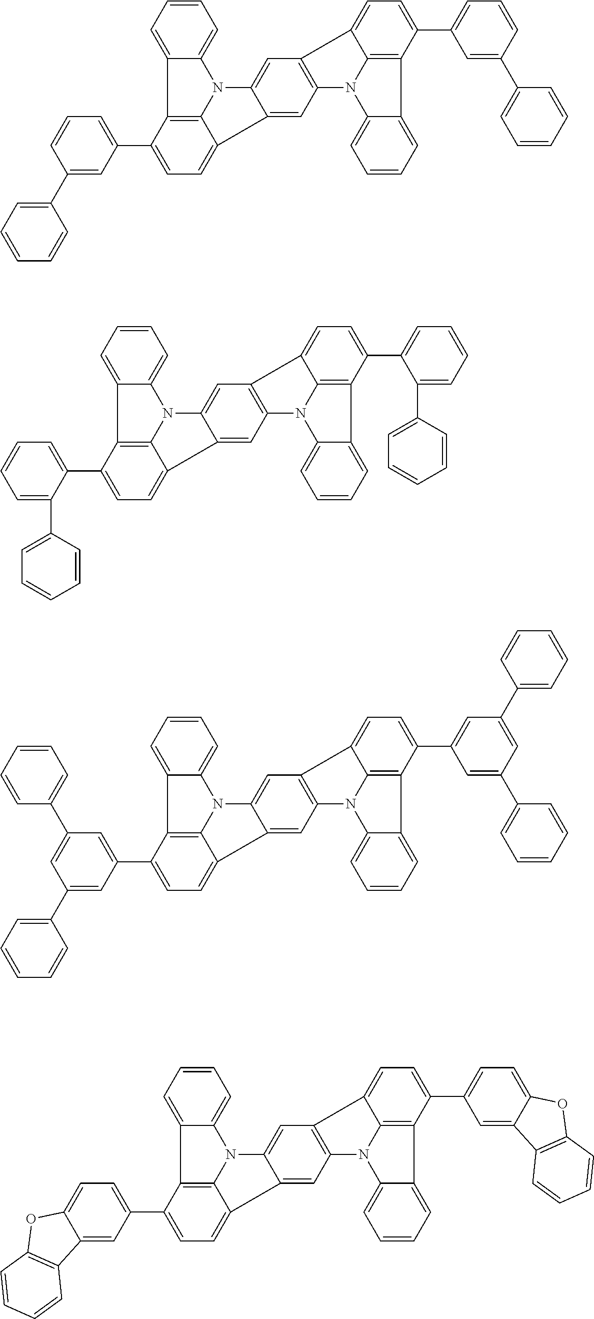 [SMILES: c1ccc(-c2cccc(-c3ccc4c5cc6c(cc5n5c7ccccc7c3c45)c3ccc(-c4cccc(-c5ccccc5)c4)c4c5ccccc5n6c34)c2)cc1 ; c1ccc(-c2ccccc2-c2ccc3c4cc5c(cc4n4c6ccccc6c2c34)c2ccc(-c3ccccc3-c3ccccc3)c3c4ccccc4n5c23)cc1 ; c1ccc(-c2cc(-c3ccccc3)cc(-c3ccc4c5cc6c(cc5n5c7ccccc7c3c45)c3ccc(-c4cc(-c5ccccc5)cc(-c5ccccc5)c4)c4c5ccccc5n6c34)c2)cc1 ; c1ccc2c(c1)oc1ccc(-c3ccc4c5cc6c(cc5n5c7ccccc7c3c45)c3ccc(-c4ccc5oc7ccccc7c5c4)c4c5ccccc5n6c34)cc12]

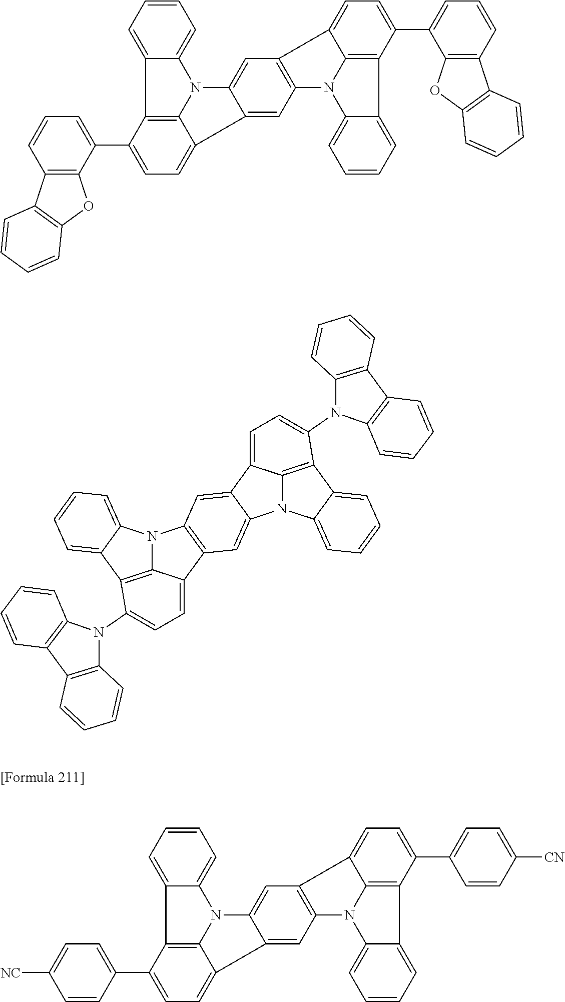 [SMILES: c1ccc2c(c1)oc1c(-c3ccc4c5cc6c(cc5n5c7ccccc7c3c45)c3ccc(-c4cccc5c4oc4ccccc45)c4c5ccccc5n6c34)cccc12 ; c1ccc2c(c1)c1ccccc1n2-c1ccc2c3cc4c(cc3n3c5ccccc5c1c23)c1ccc(-n2c3ccccc3c3ccccc32)c2c3ccccc3n4c12 ; N#Cc1ccc(-c2ccc3c4cc5c(cc4n4c6ccccc6c2c34)c2ccc(-c3ccc(C#N)cc3)c3c4ccccc4n5c23)cc1]

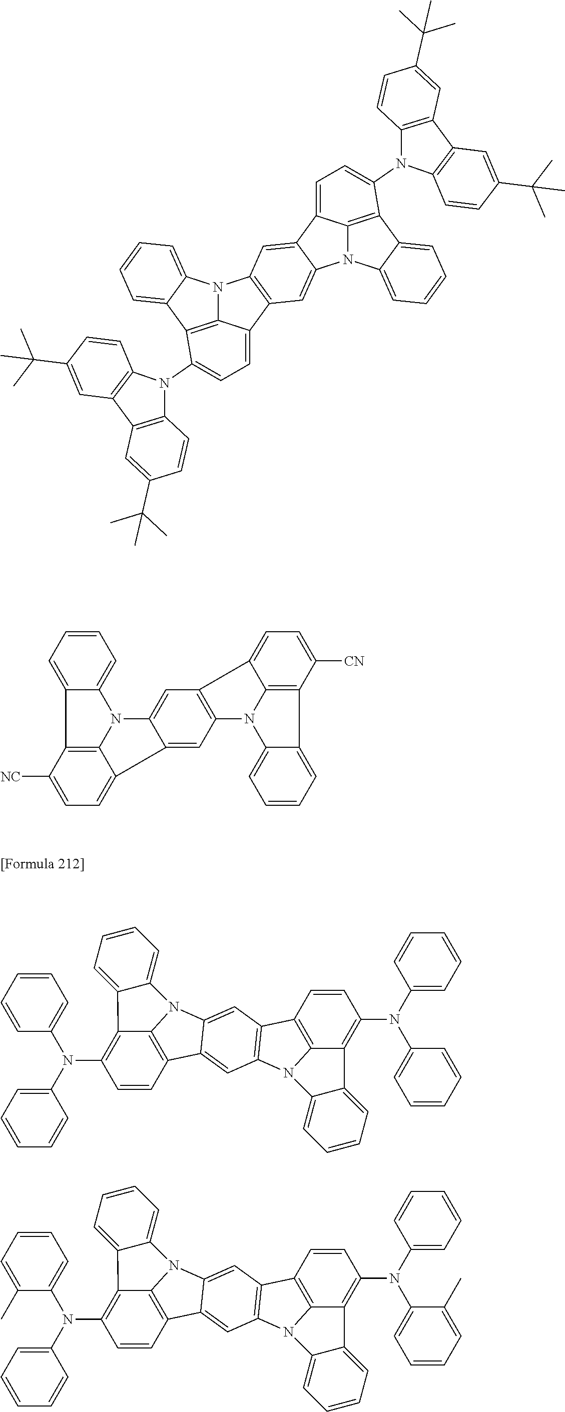 [SMILES: CC(C)(C)c1ccc2c(c1)c1cc(C(C)(C)C)ccc1n2-c1ccc2c3cc4c(cc3n3c5ccccc5c1c23)c1ccc(-n2c3ccc(C(C)(C)C)cc3c3cc(C(C)(C)C)ccc32)c2c3ccccc3n4c12 ; N#Cc1ccc2c3cc4c(cc3n3c5ccccc5c1c23)c1ccc(C#N)c2c3ccccc3n4c12 ; c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12]

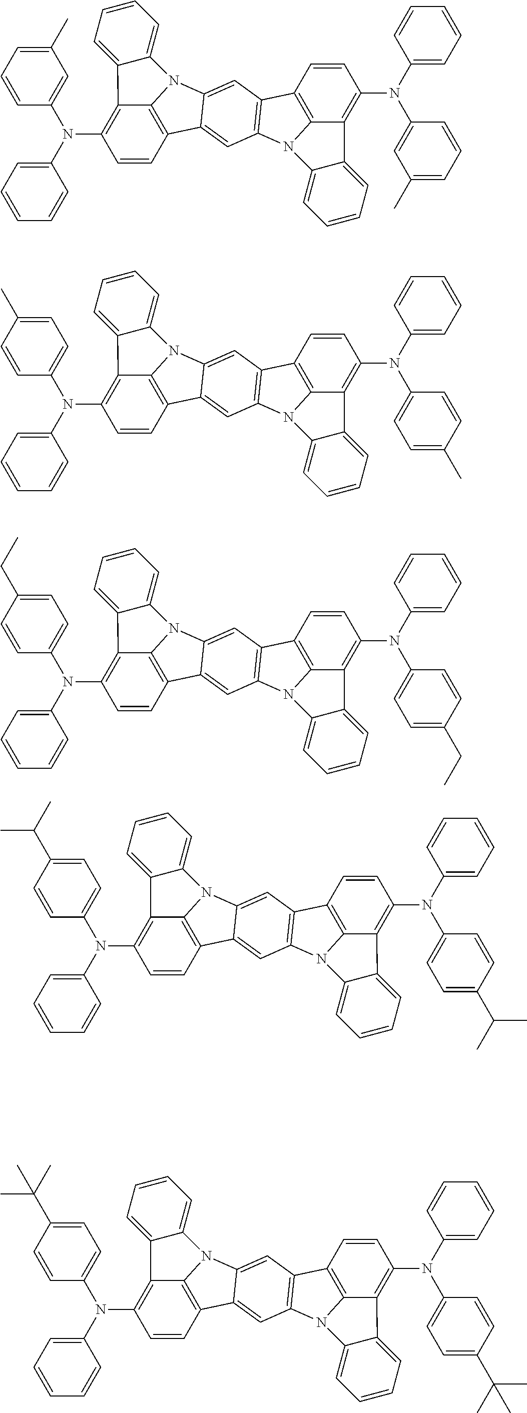

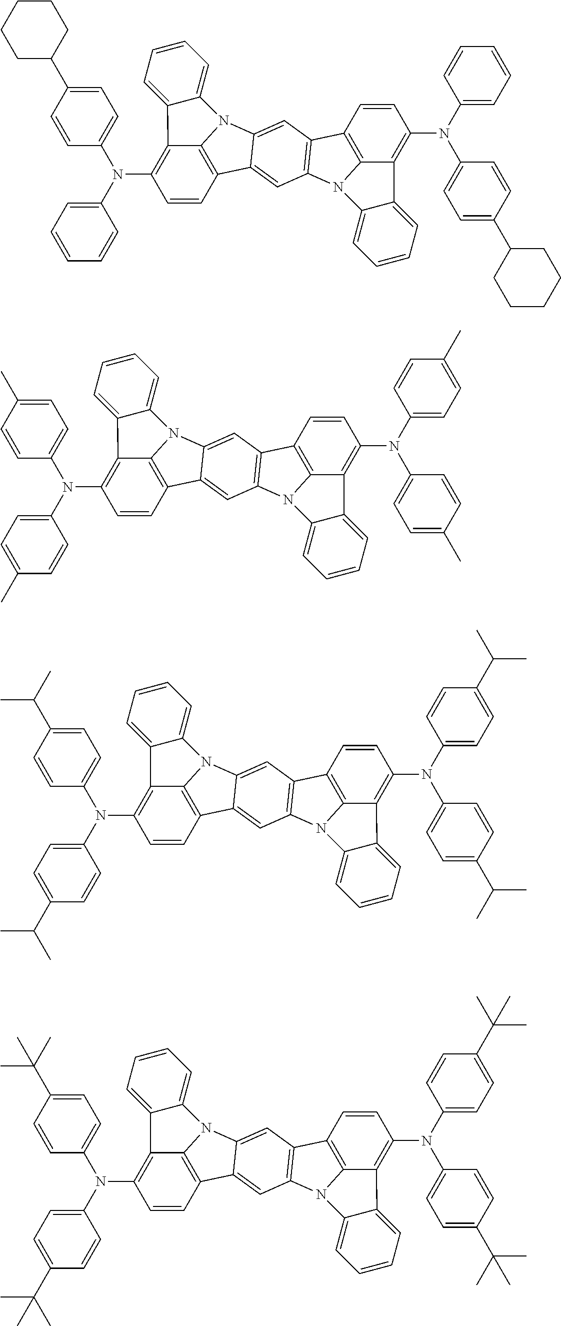

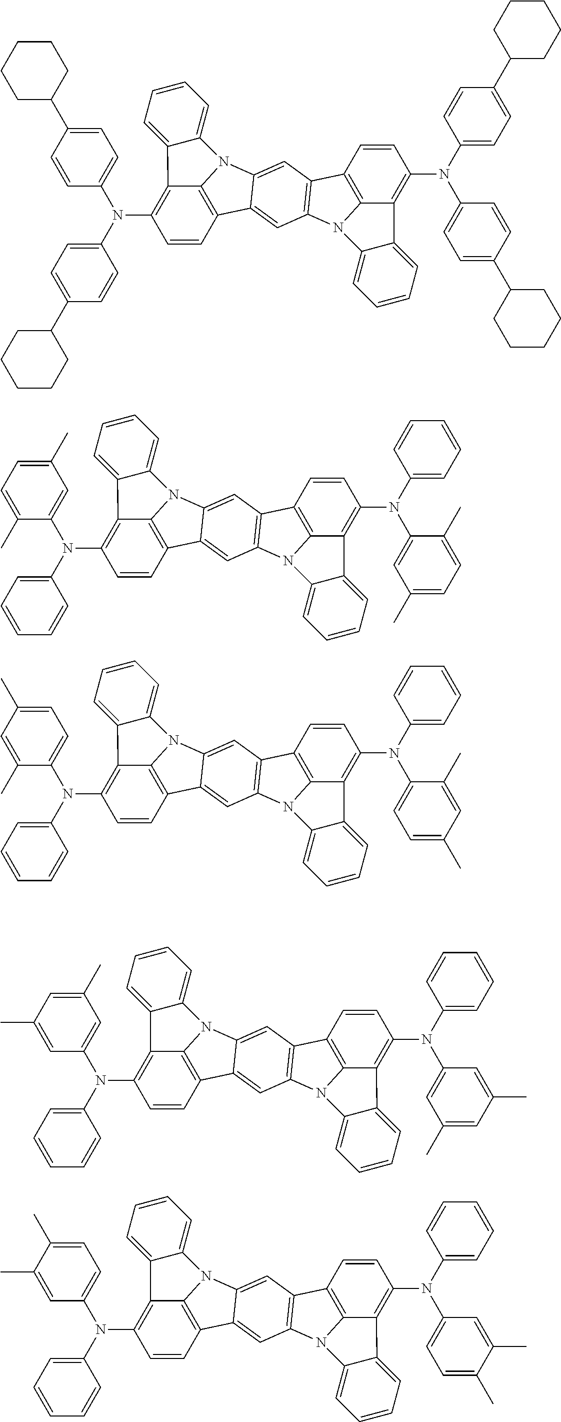

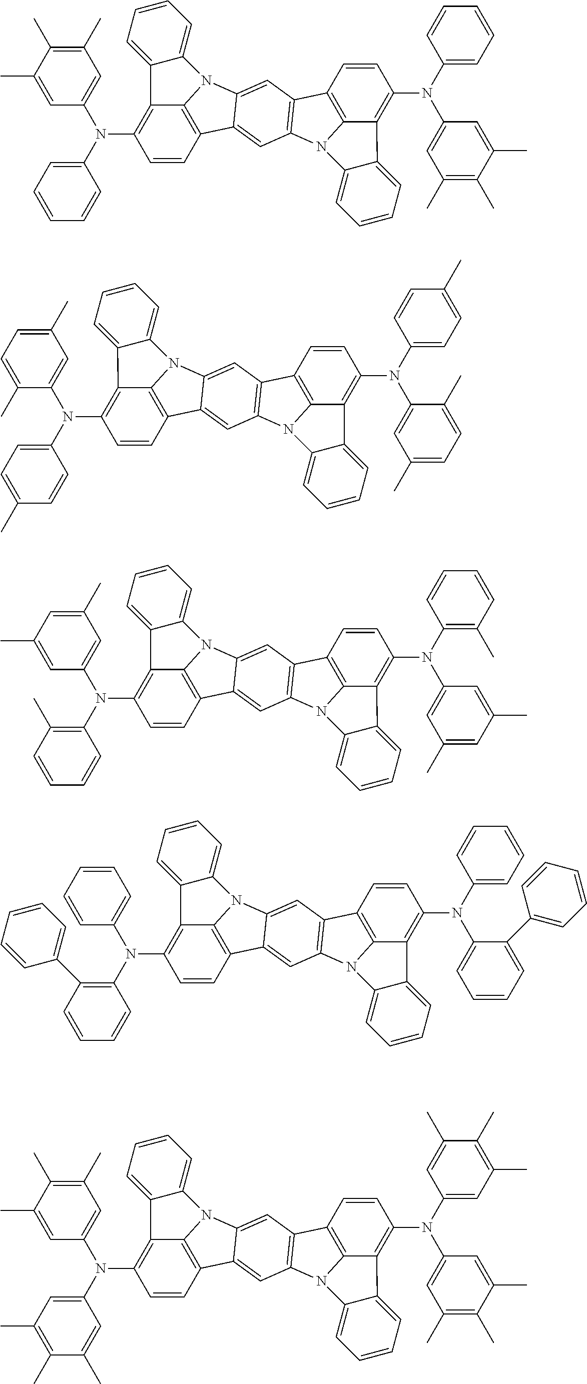

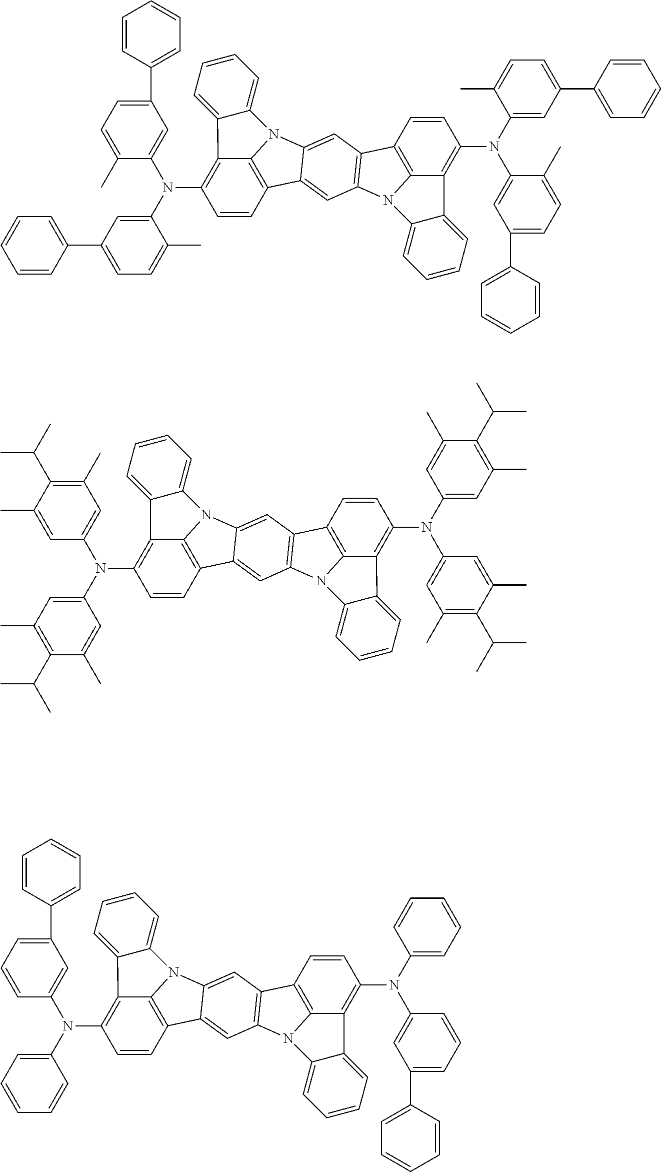 [SMILES: Cc1ccc(-c2ccccc2)cc1N(c1cc(-c2ccccc2)ccc1C)c1ccc2c3cc4c(cc3n3c5ccccc5c1c23)c1ccc(N(c2cc(-c3ccccc3)ccc2C)c2cc(-c3ccccc3)ccc2C)c2c3ccccc3n4c12 ; Cc1cc(N(c2cc(C)c(C(C)C)c(C)c2)c2ccc3c4cc5c(cc4n4c6ccccc6c2c34)c2ccc(N(c3cc(C)c(C(C)C)c(C)c3)c3cc(C)c(C(C)C)c(C)c3)c3c4ccccc4n5c23)cc(C)c1C(C)C ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1]

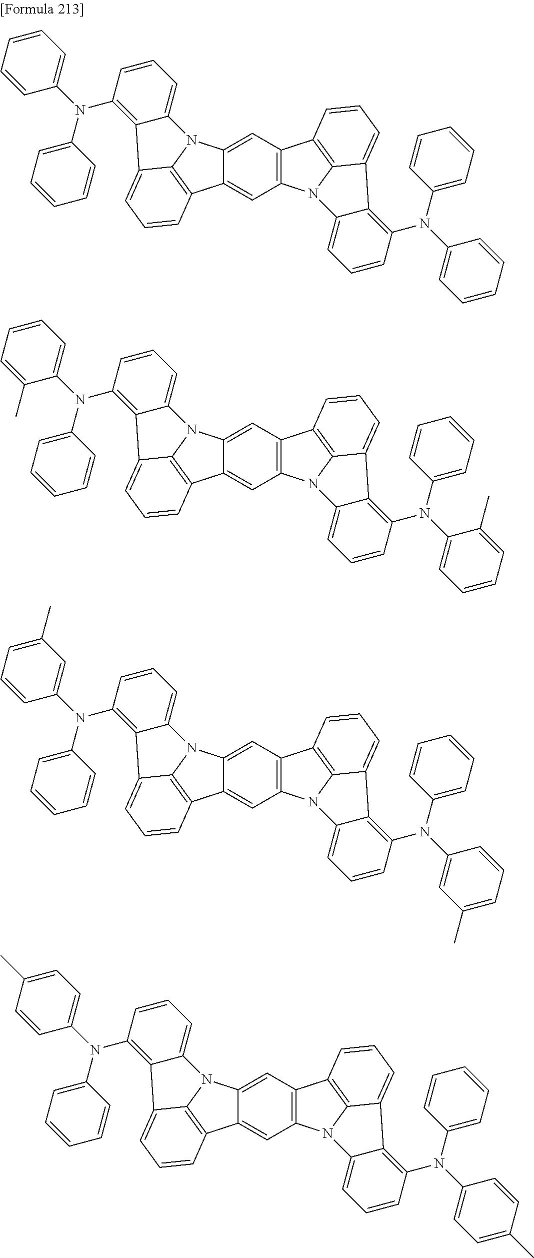

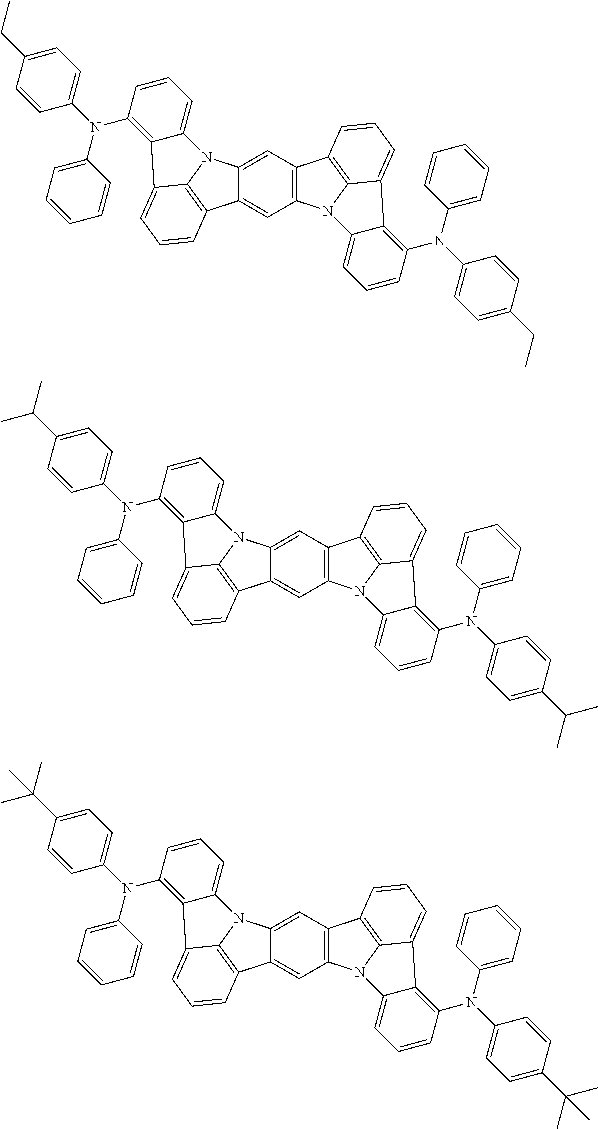

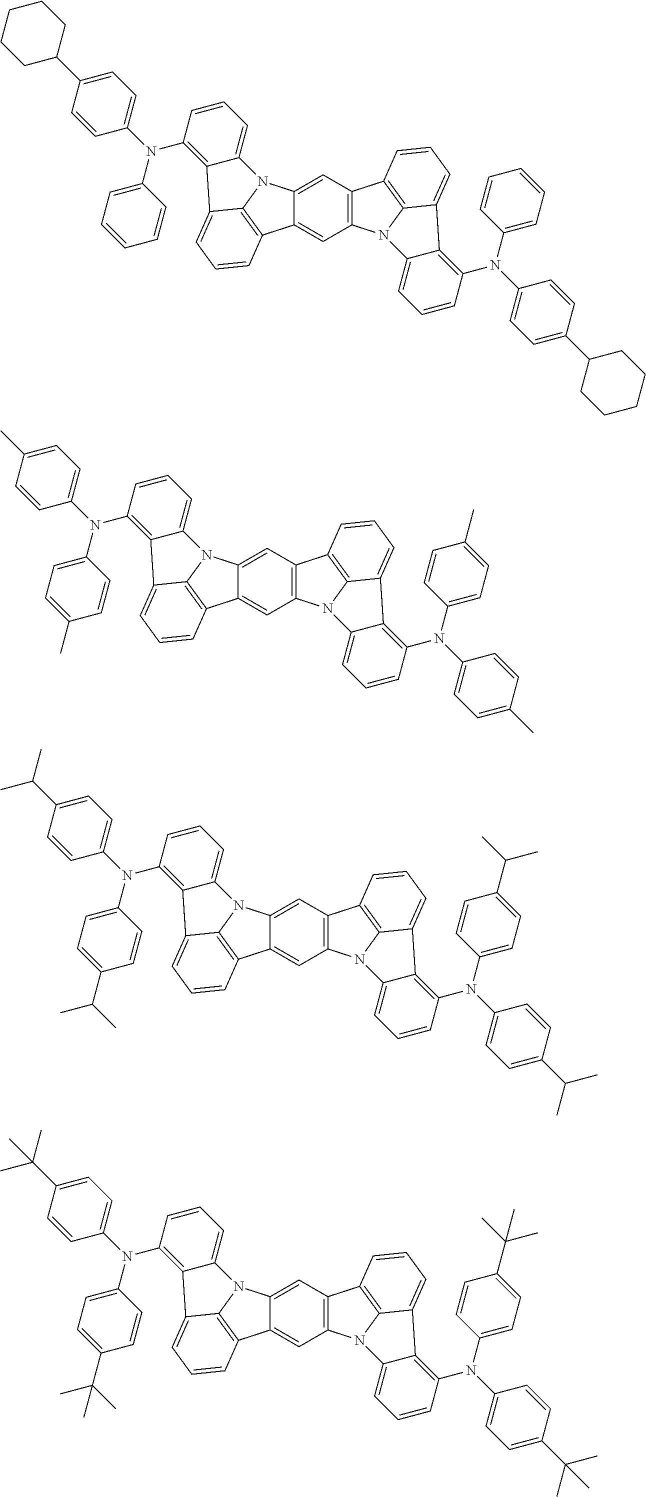

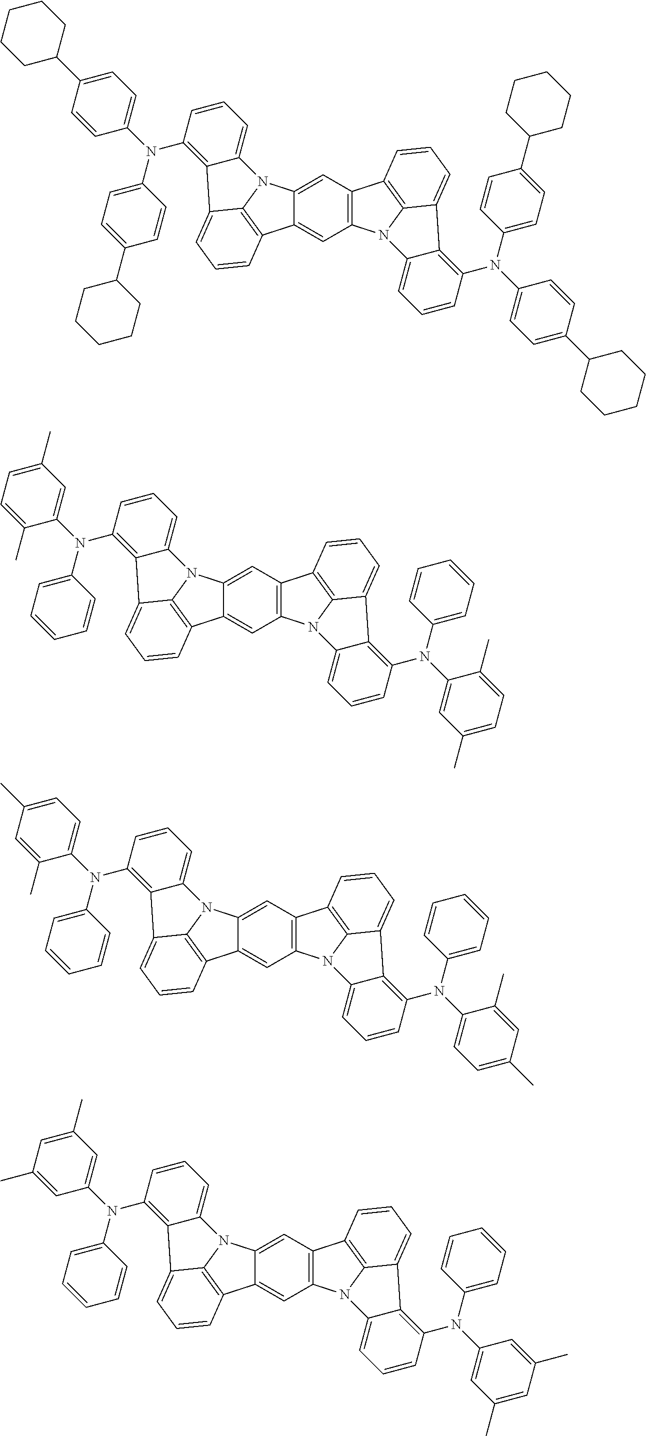

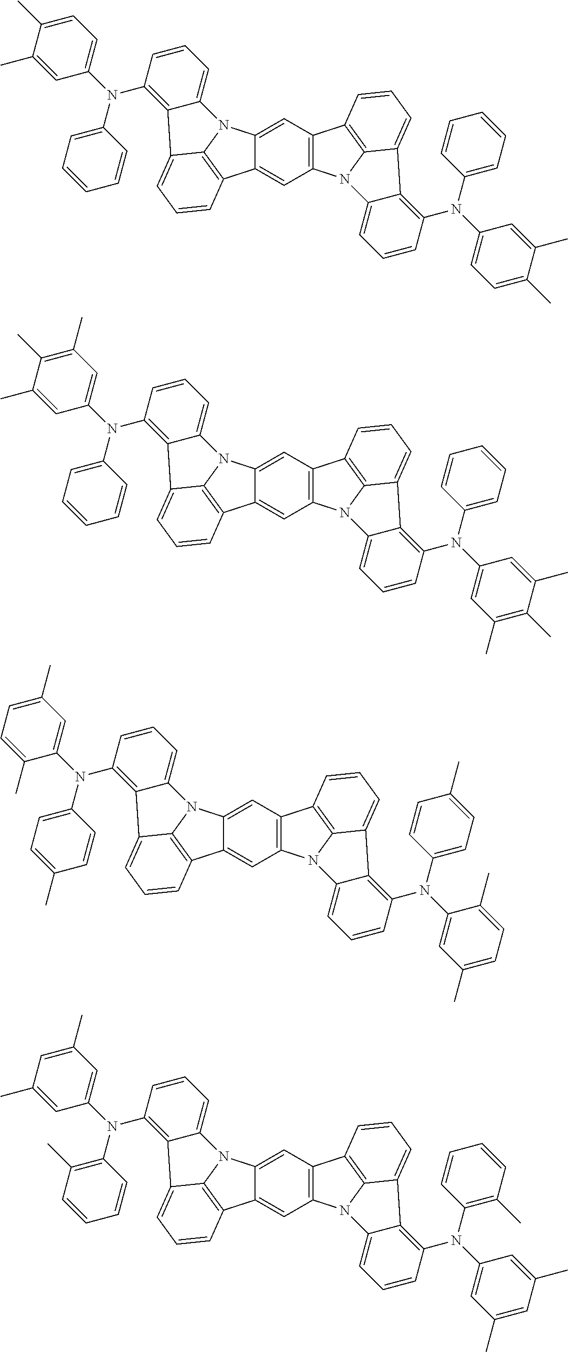

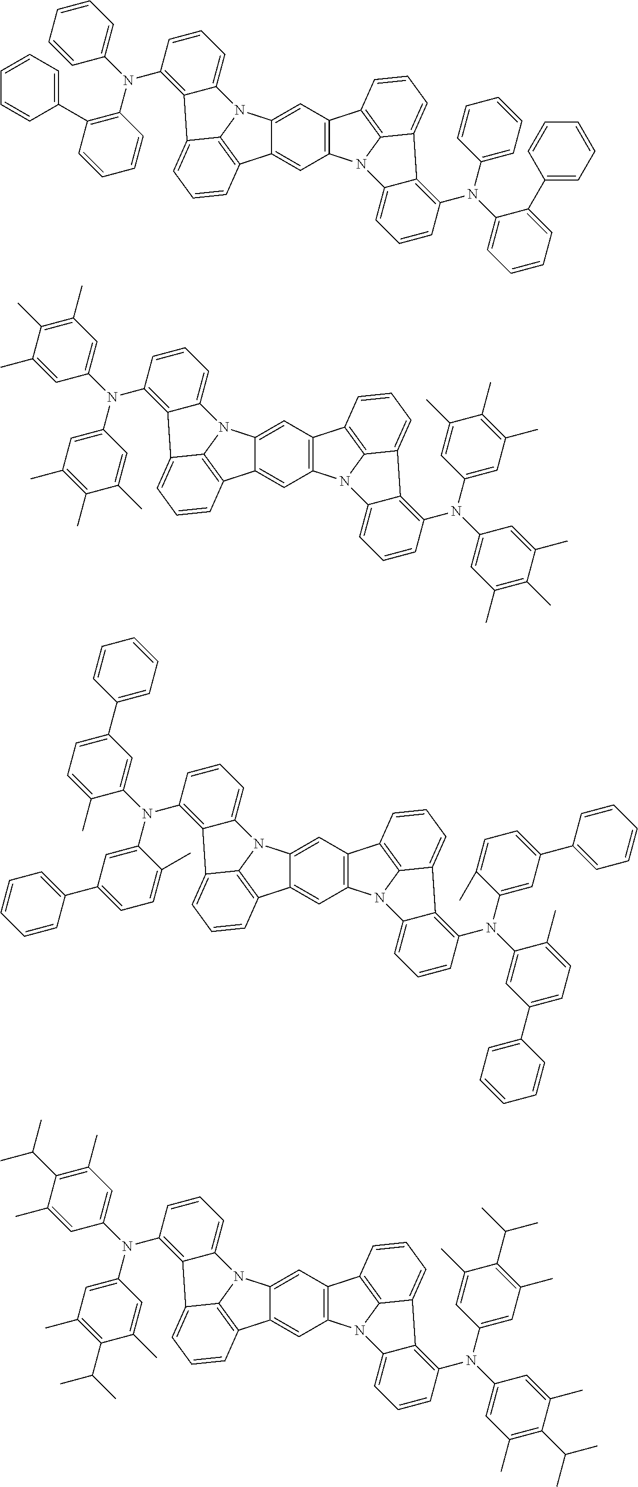

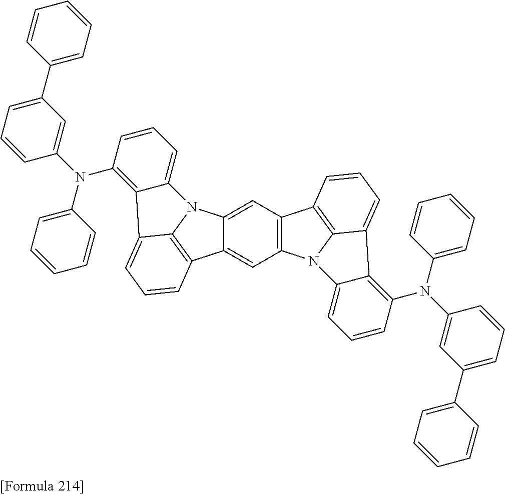 [SMILES: c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1]

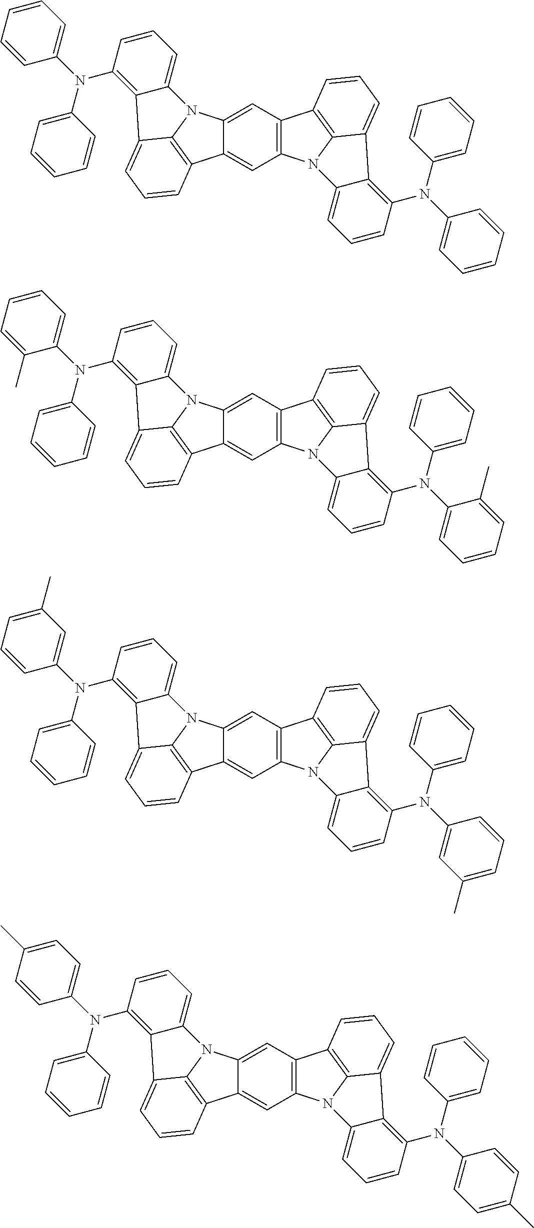

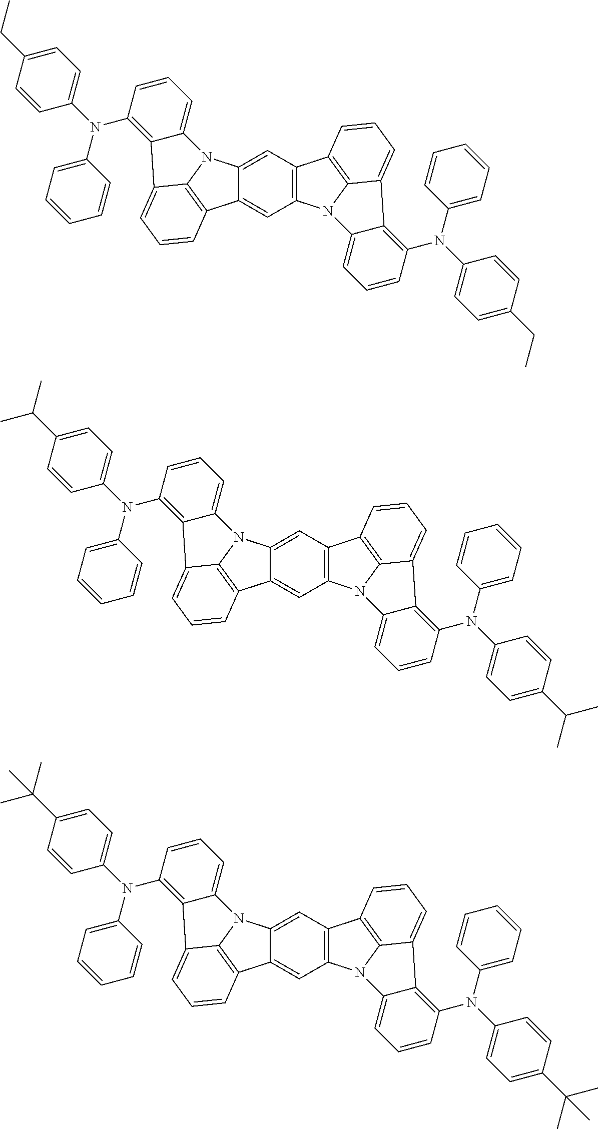

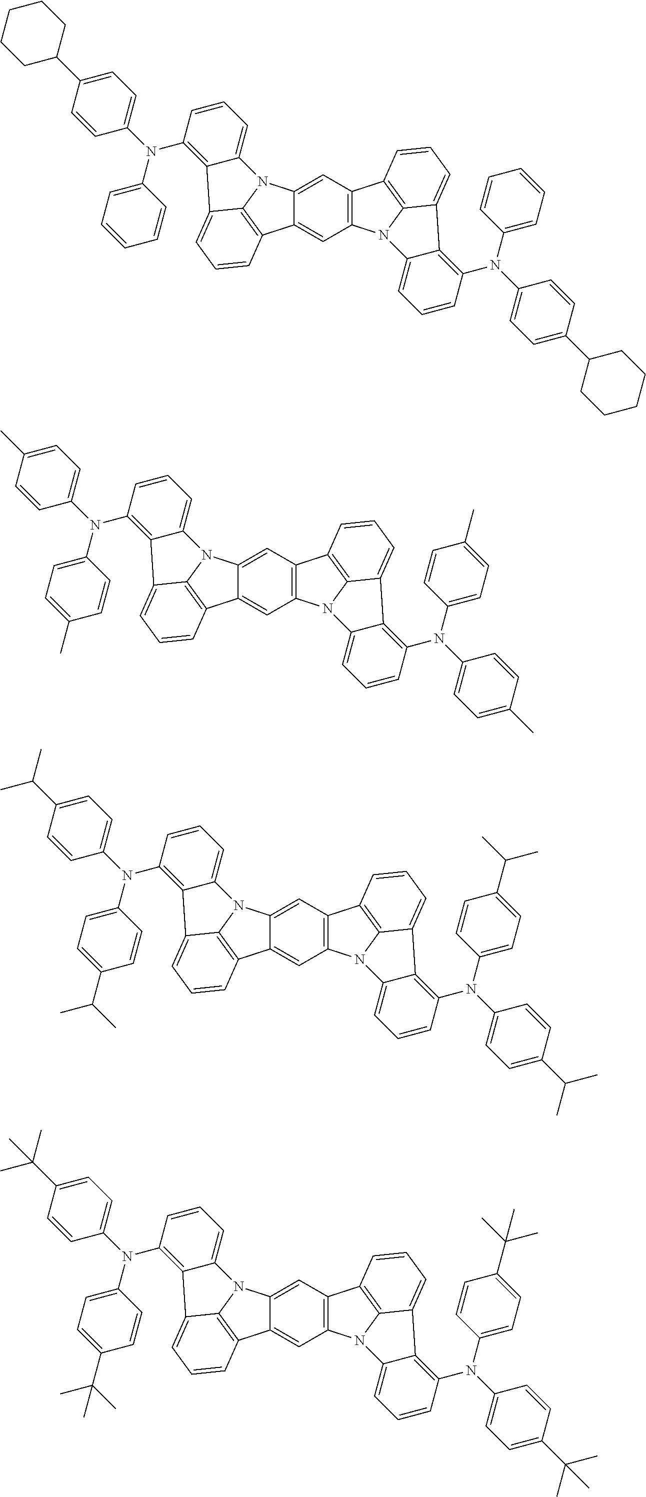

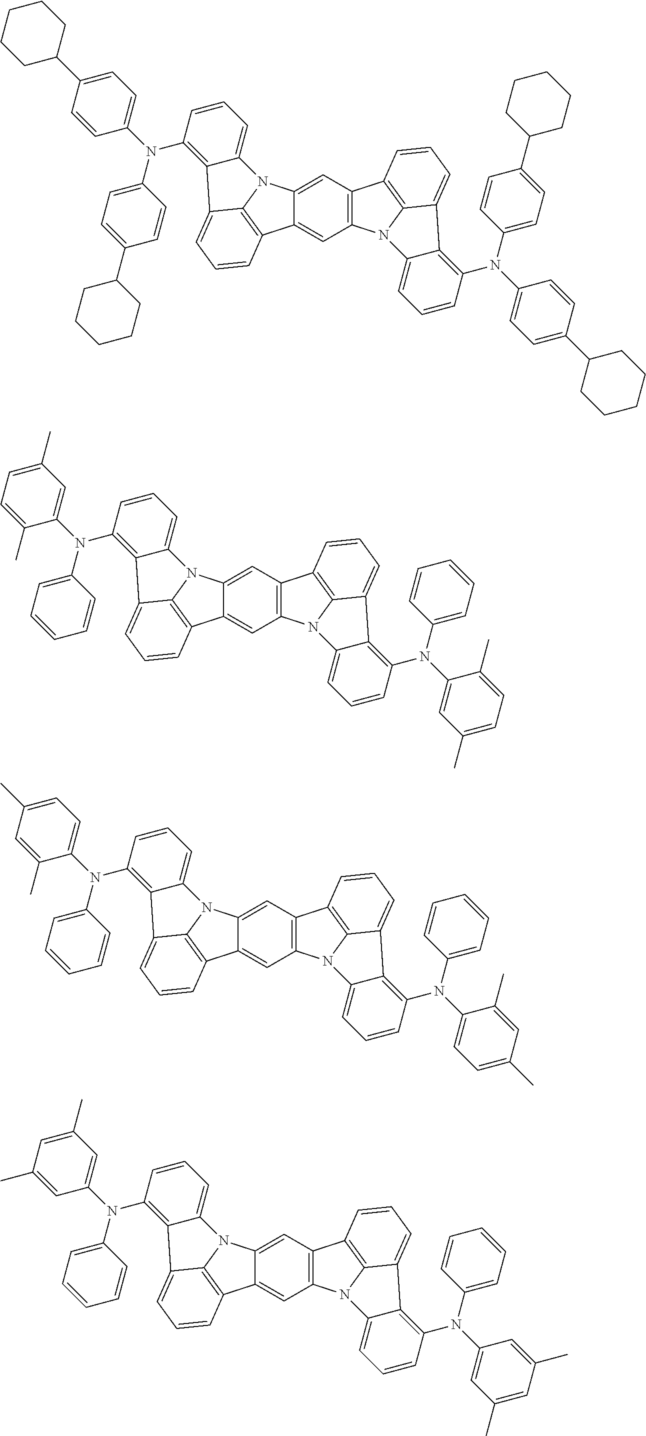

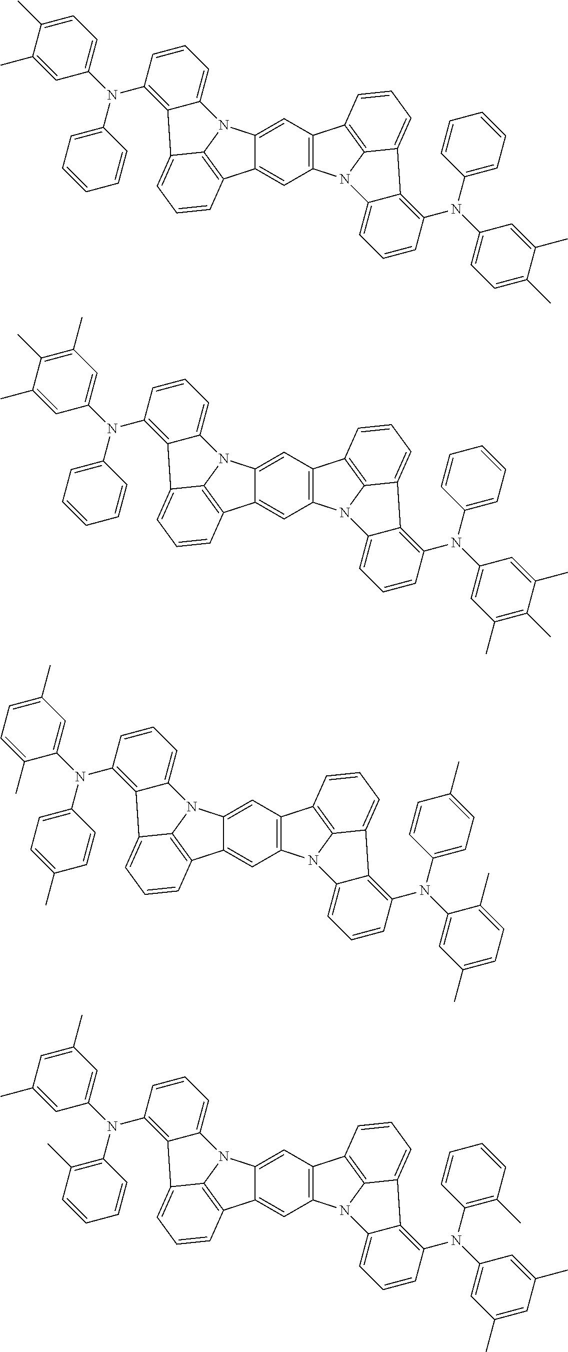

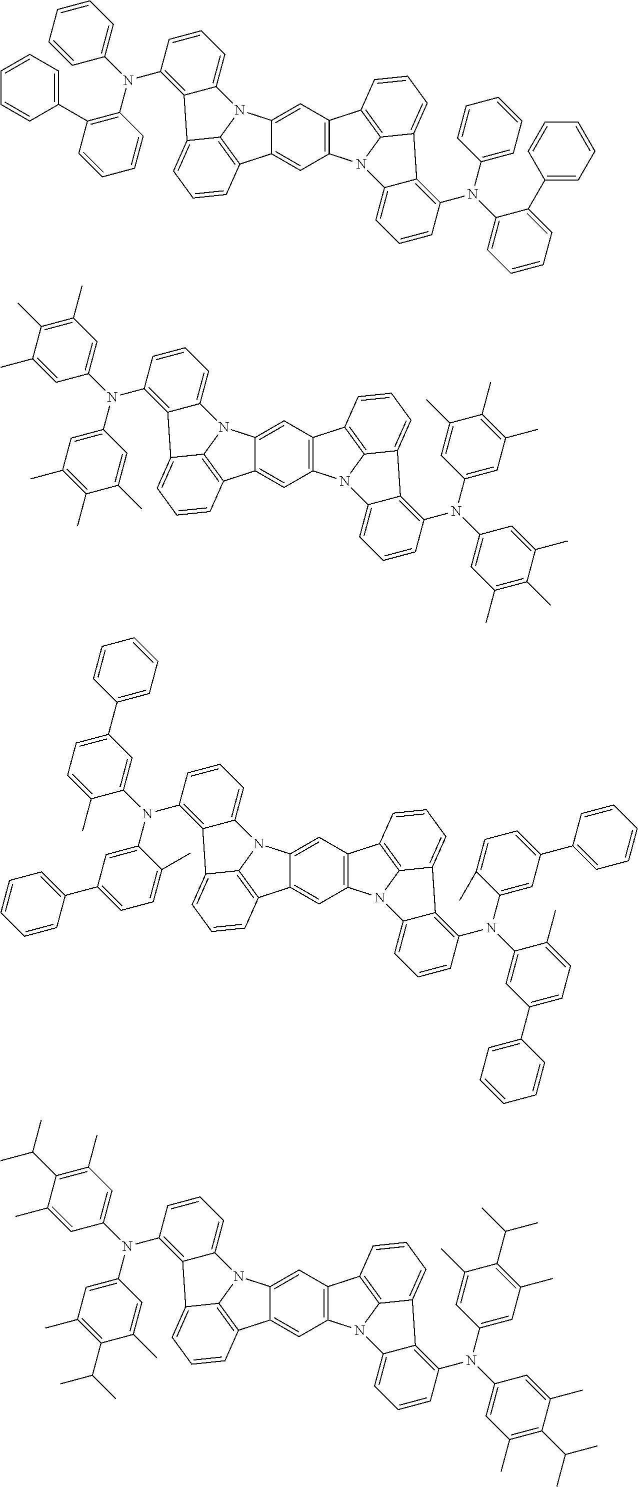

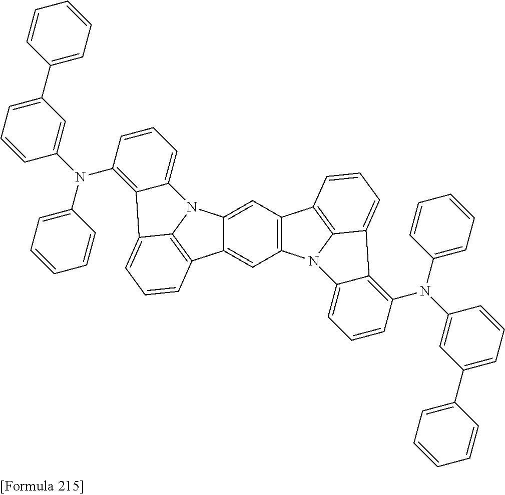 [SMILES: c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1]

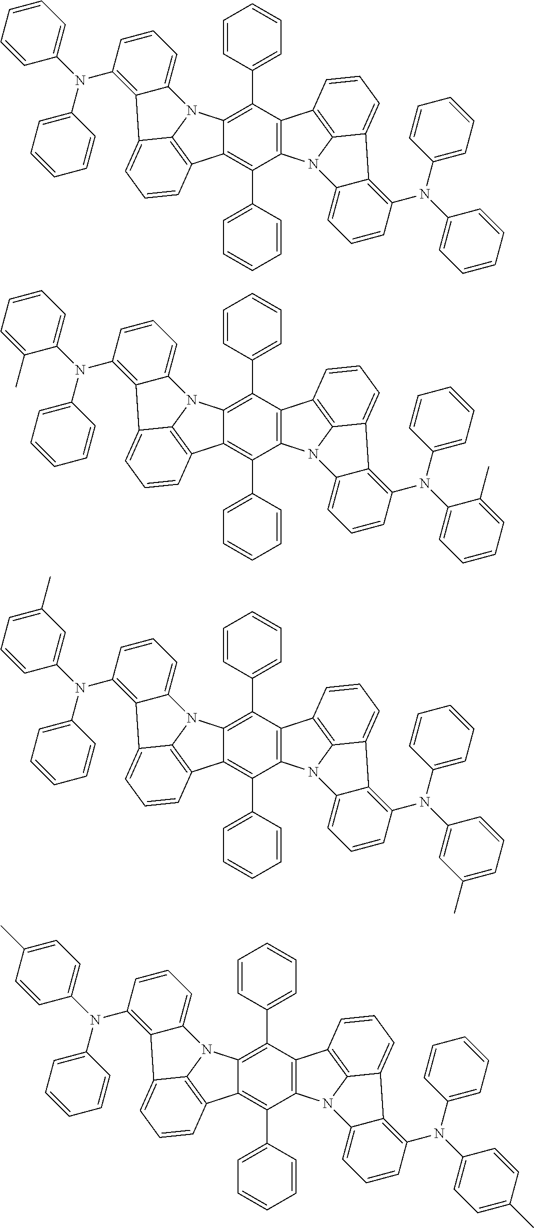

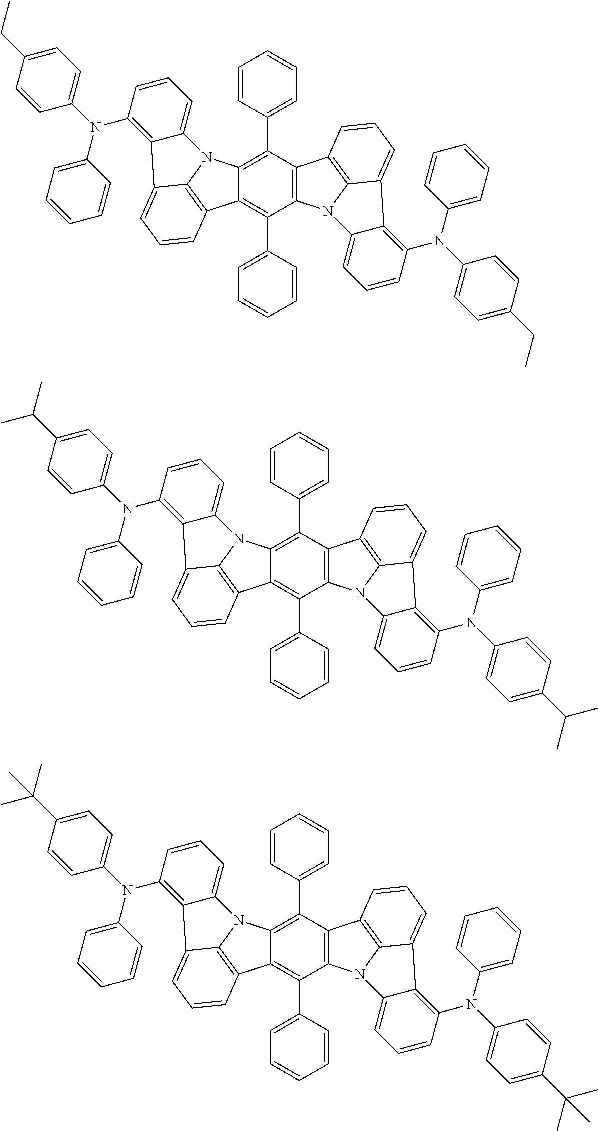

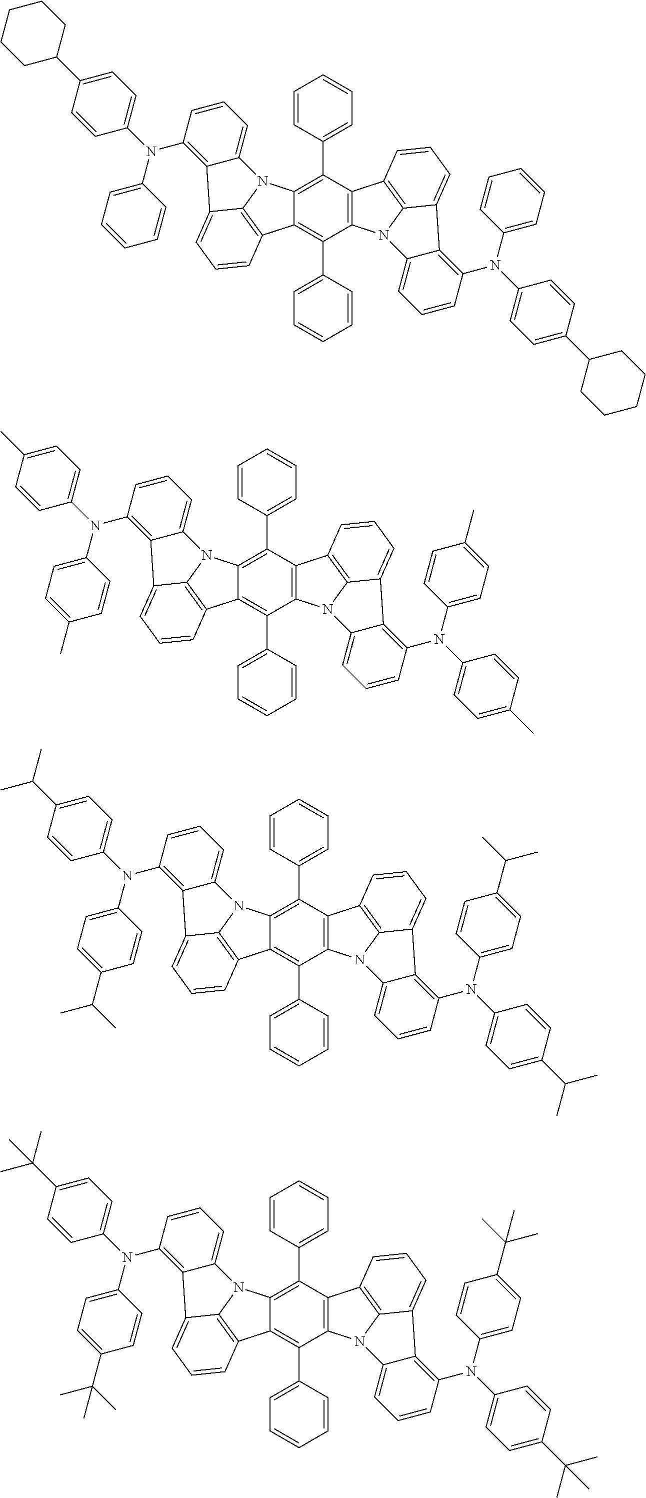

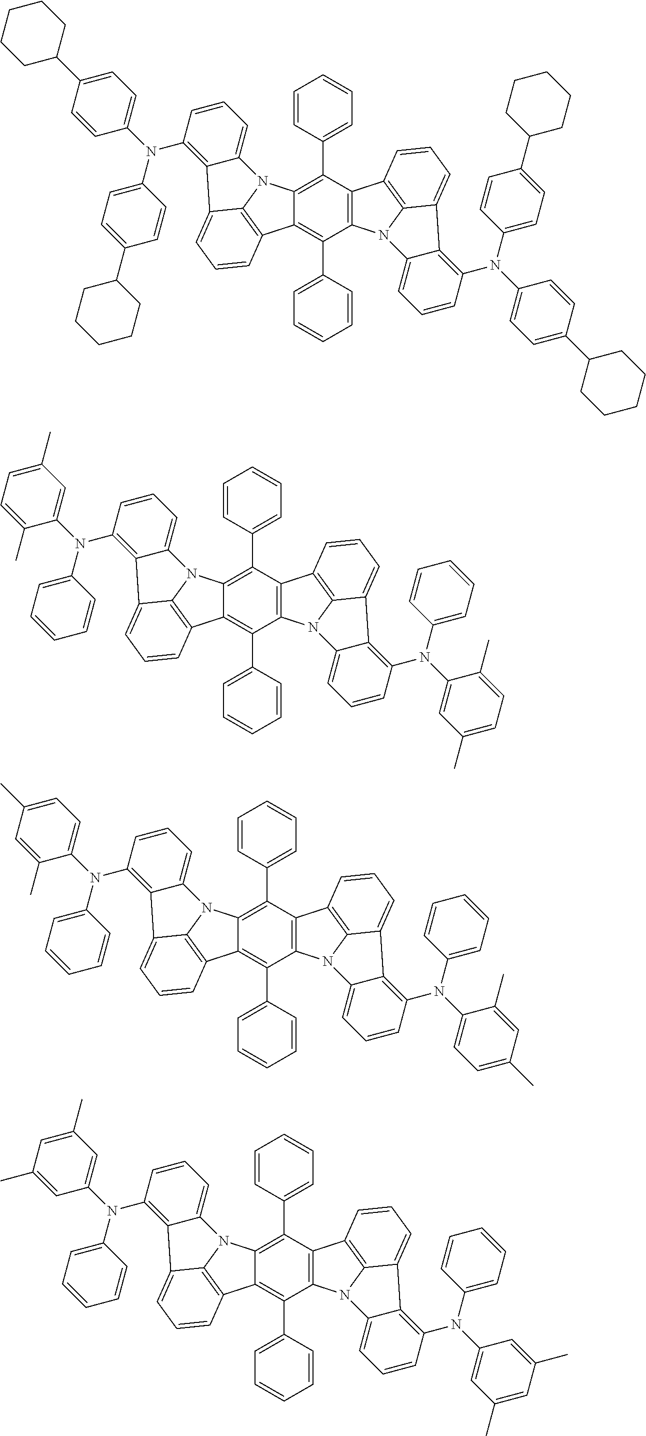

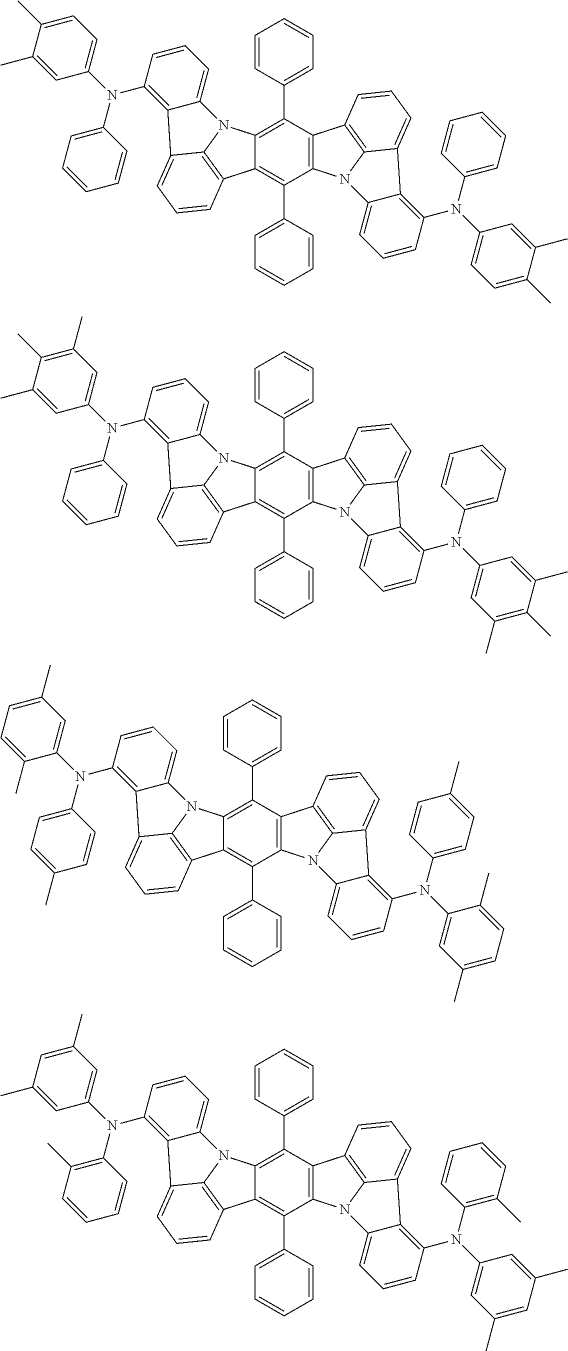

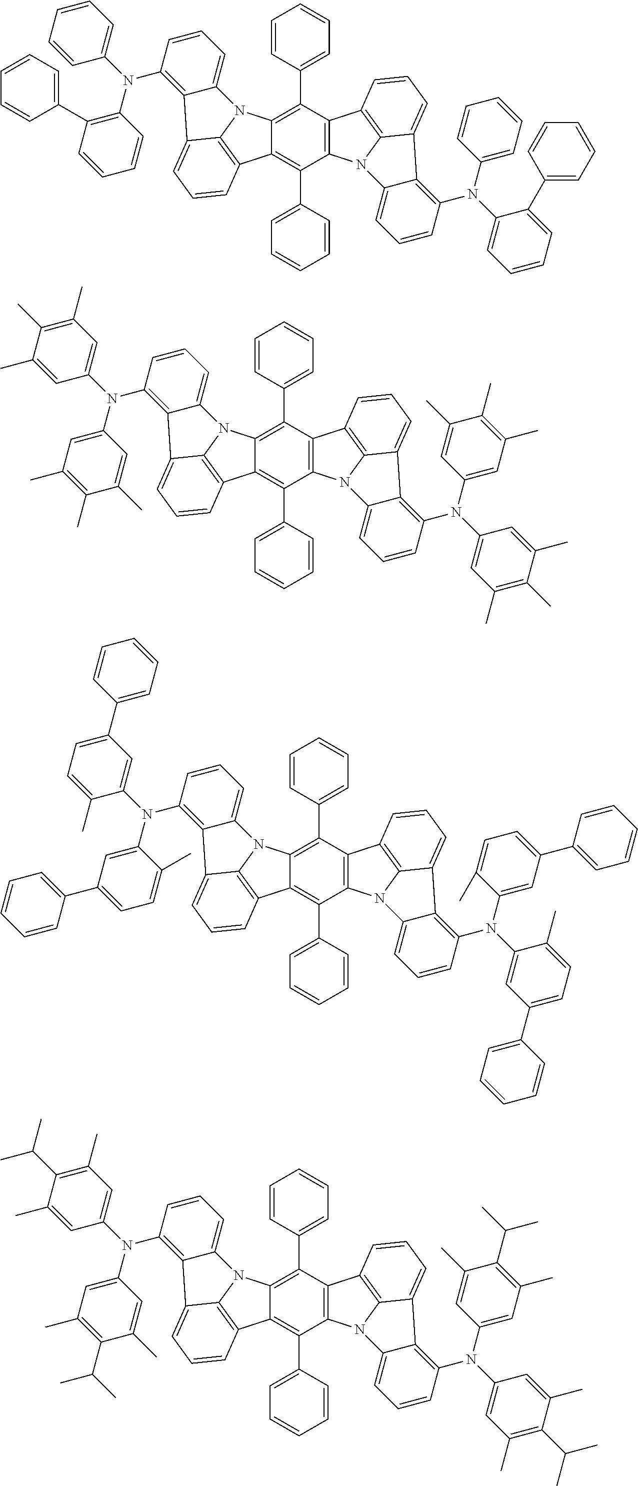

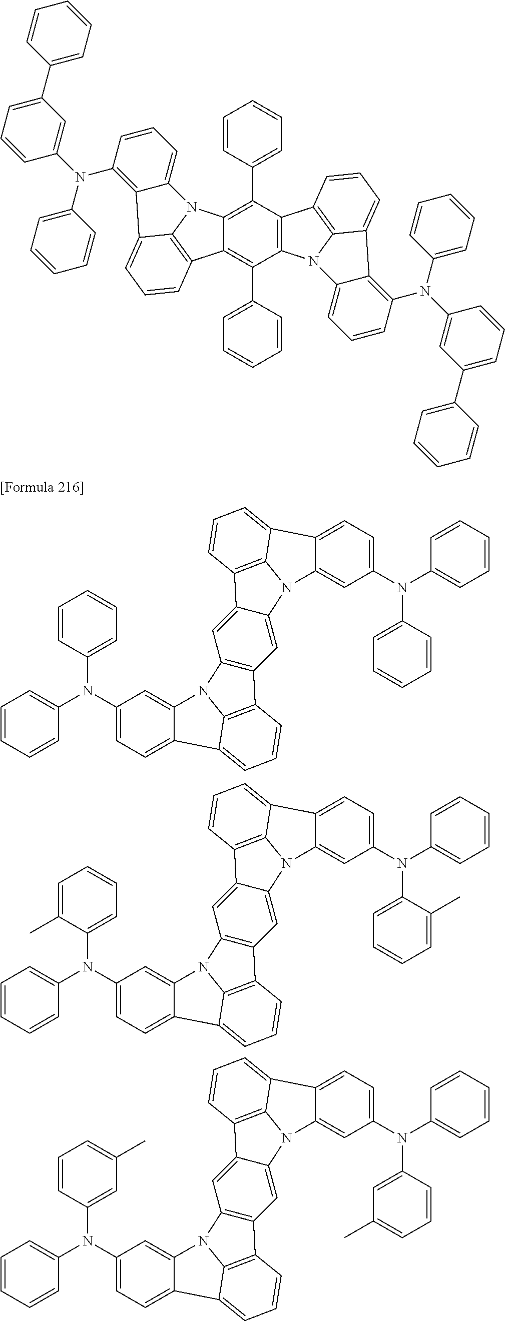

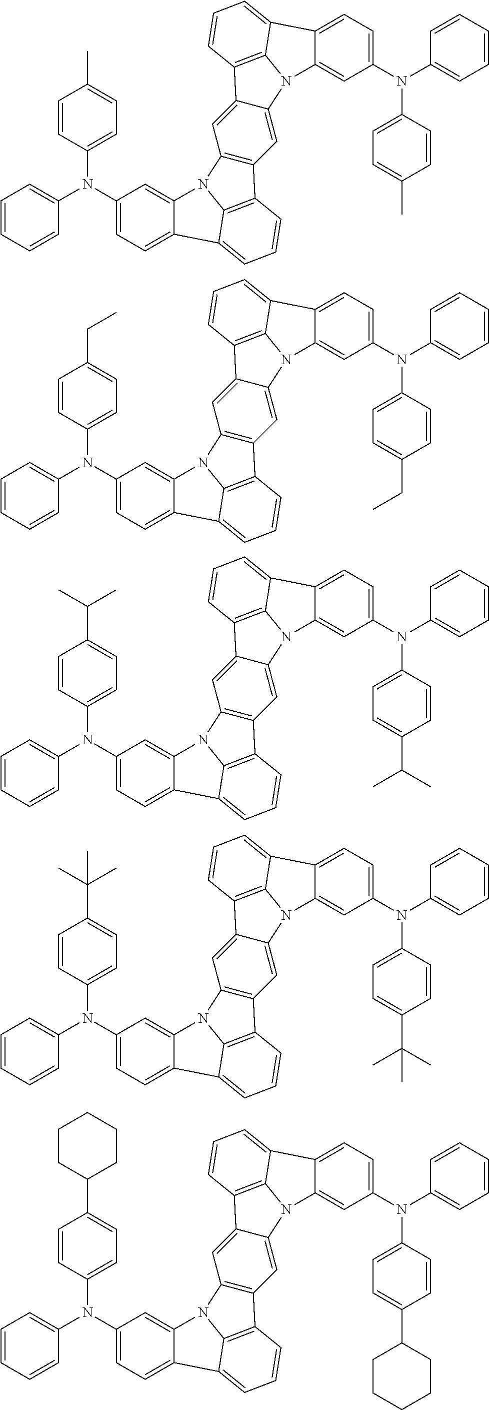

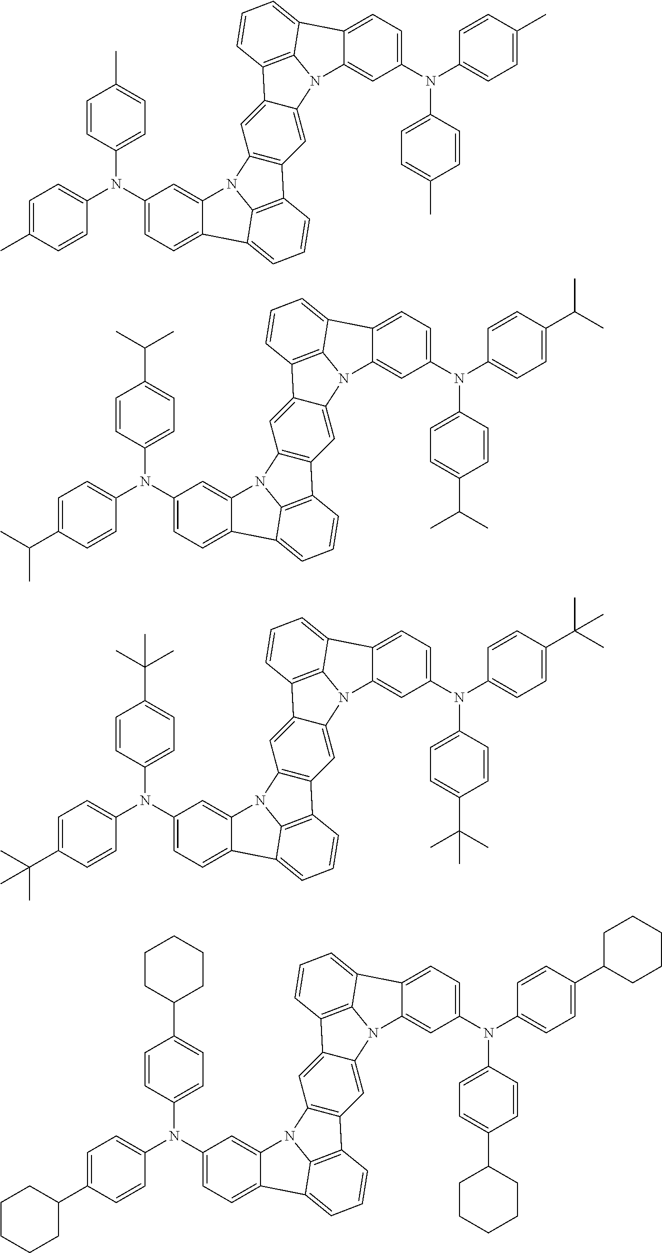

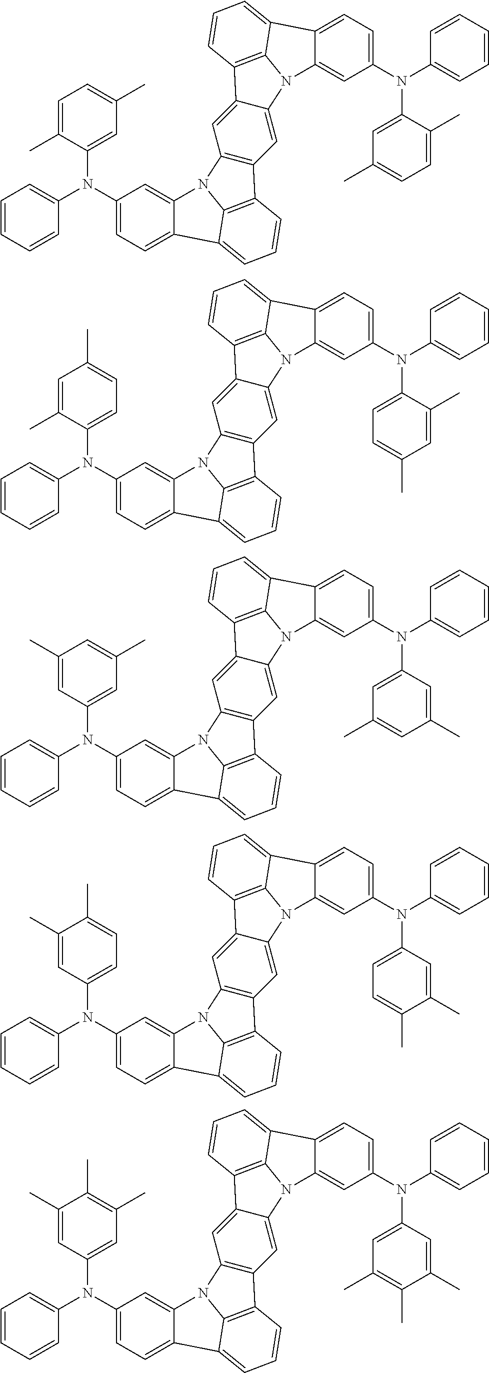

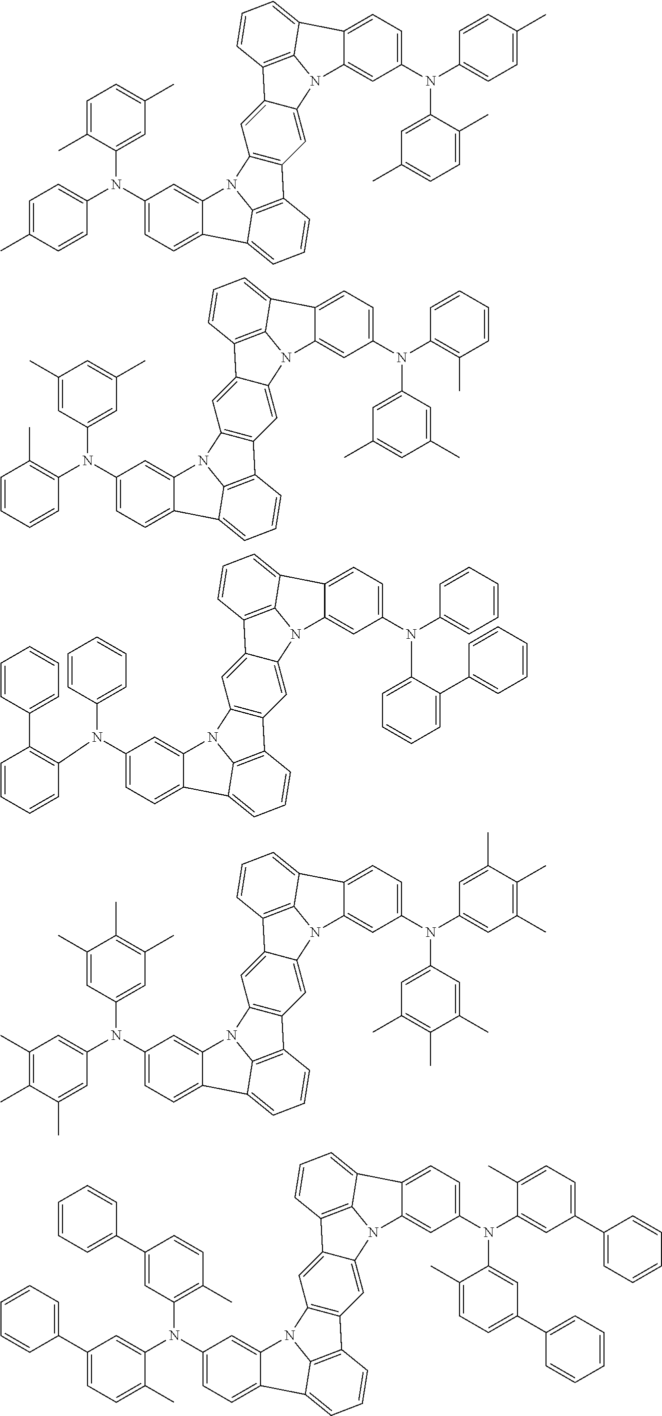

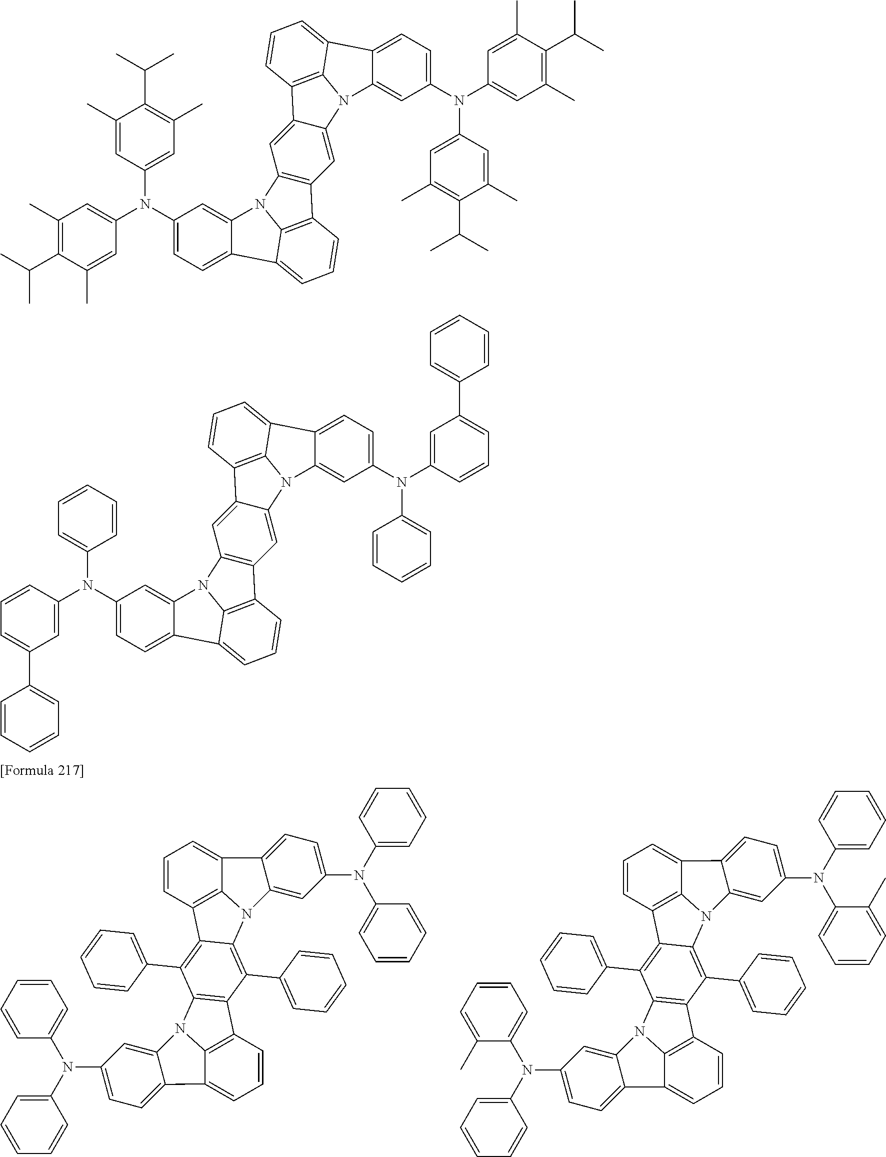

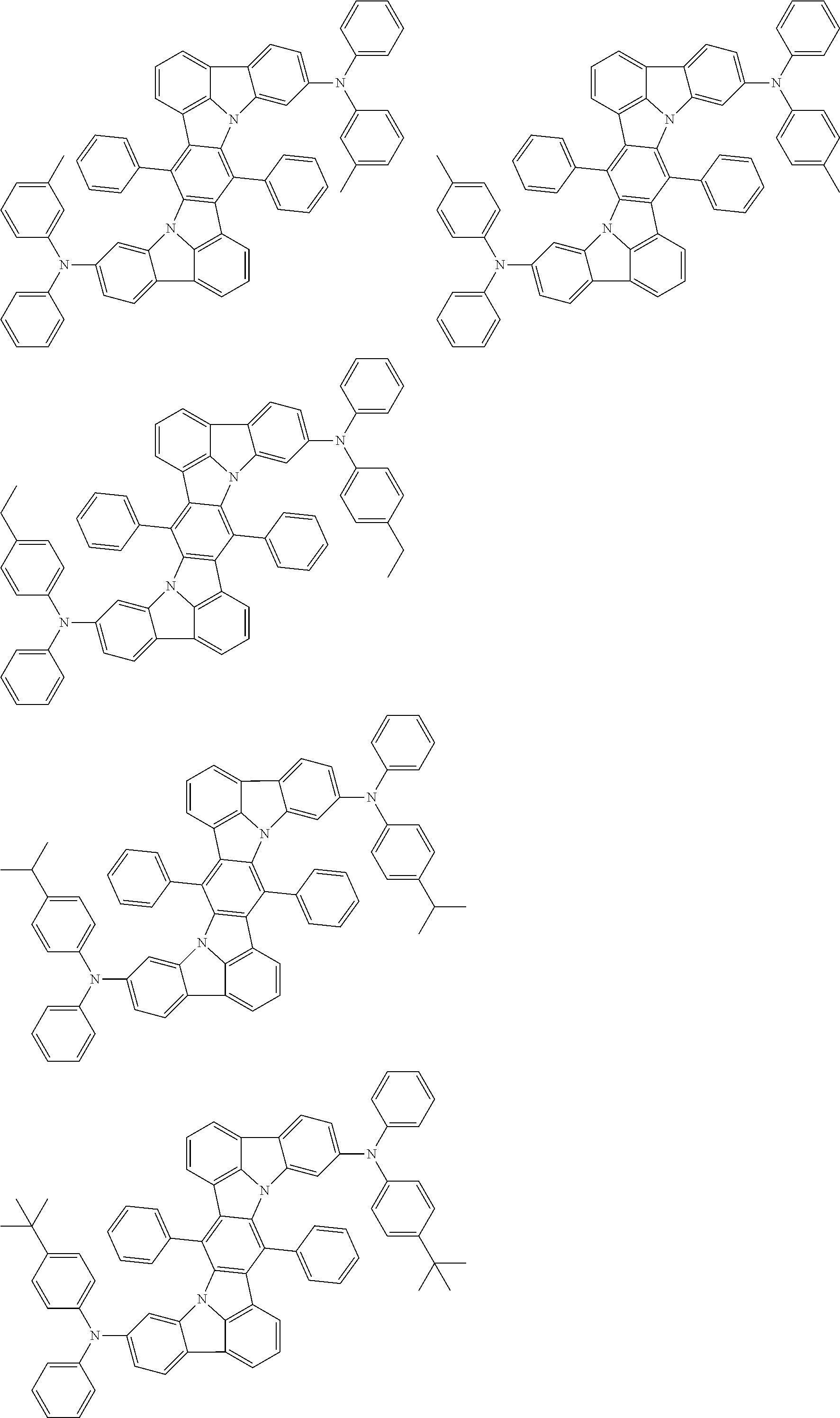

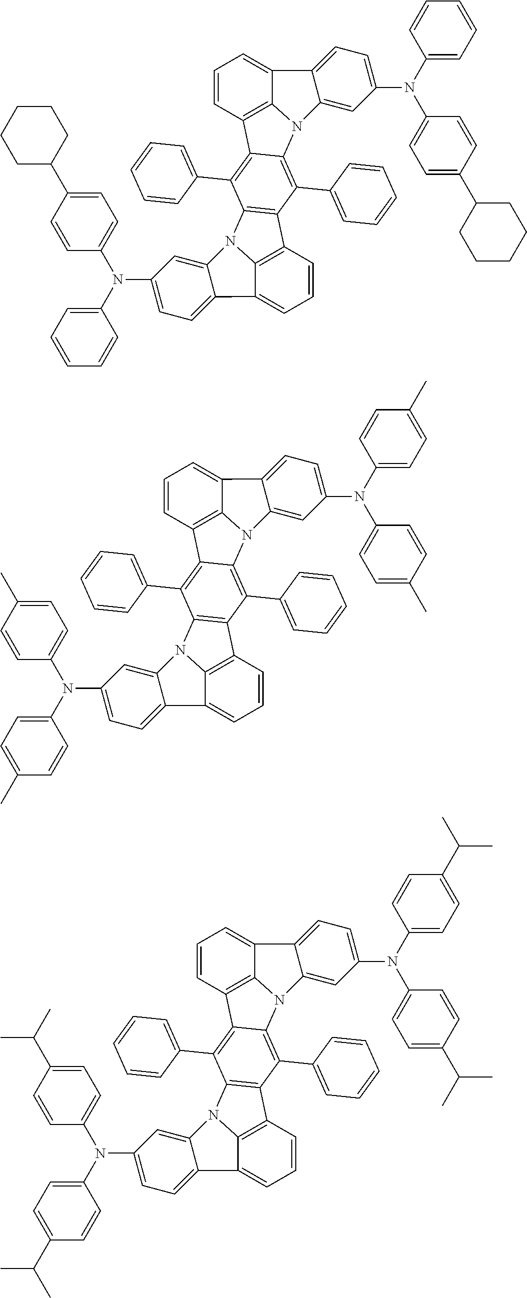

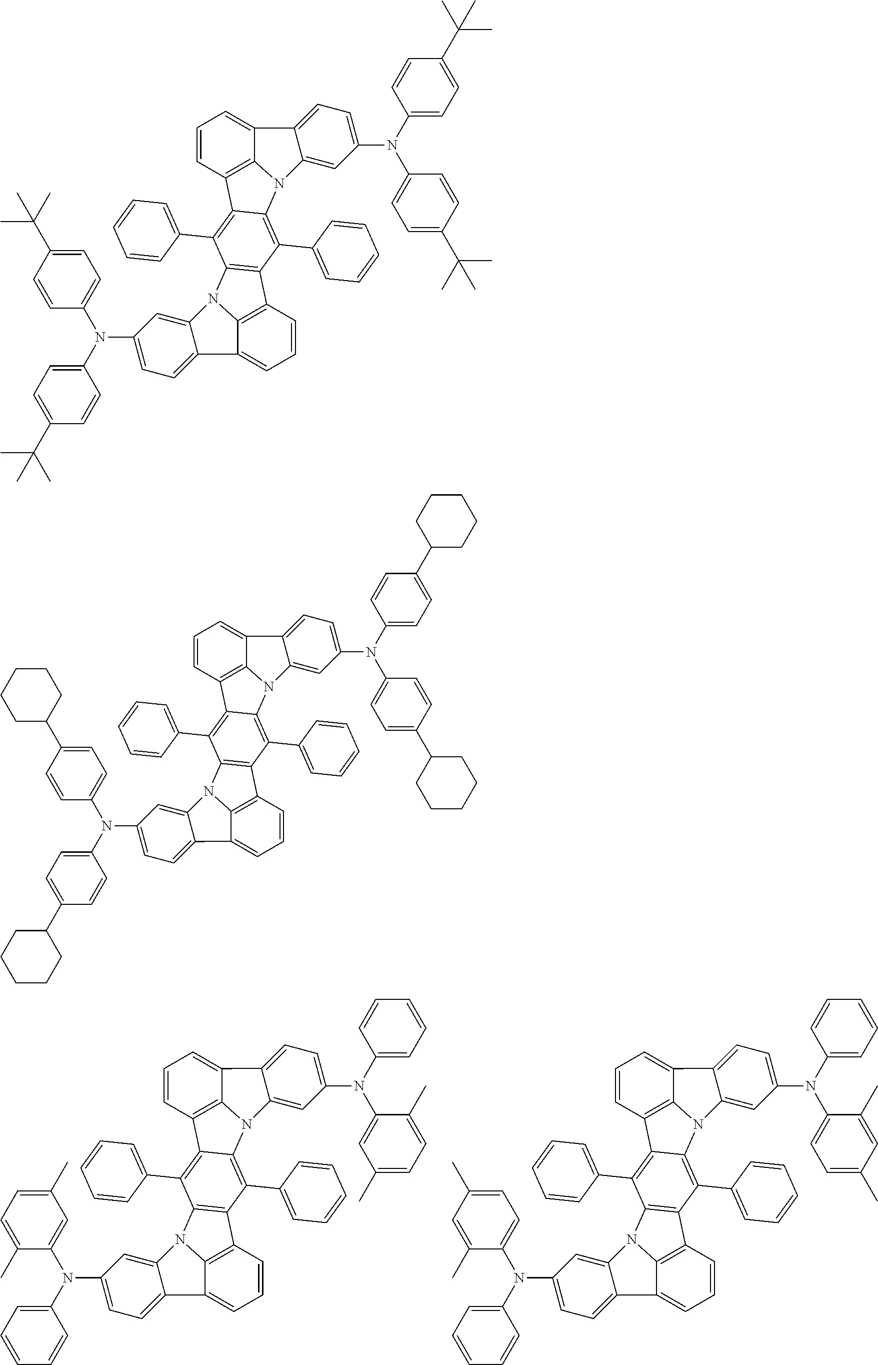

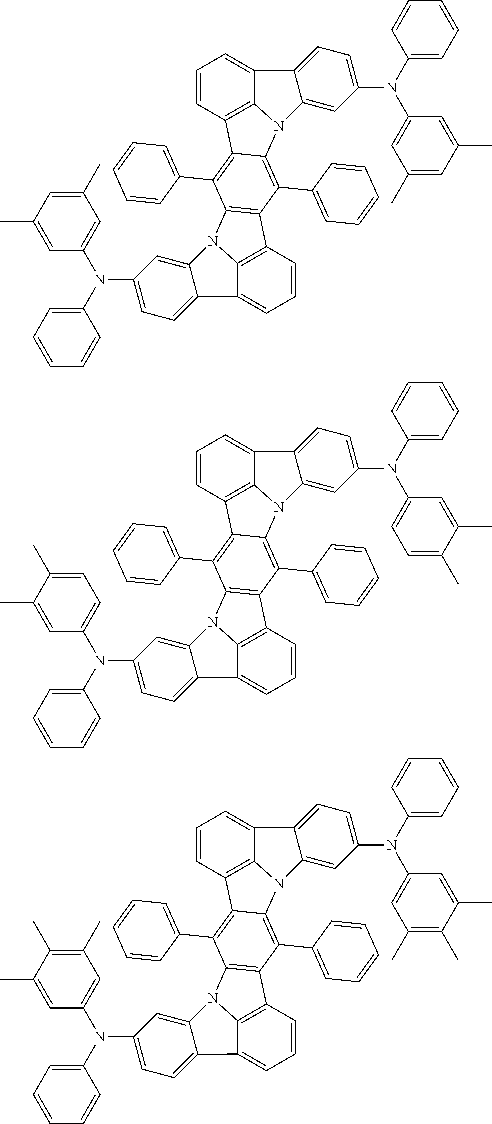

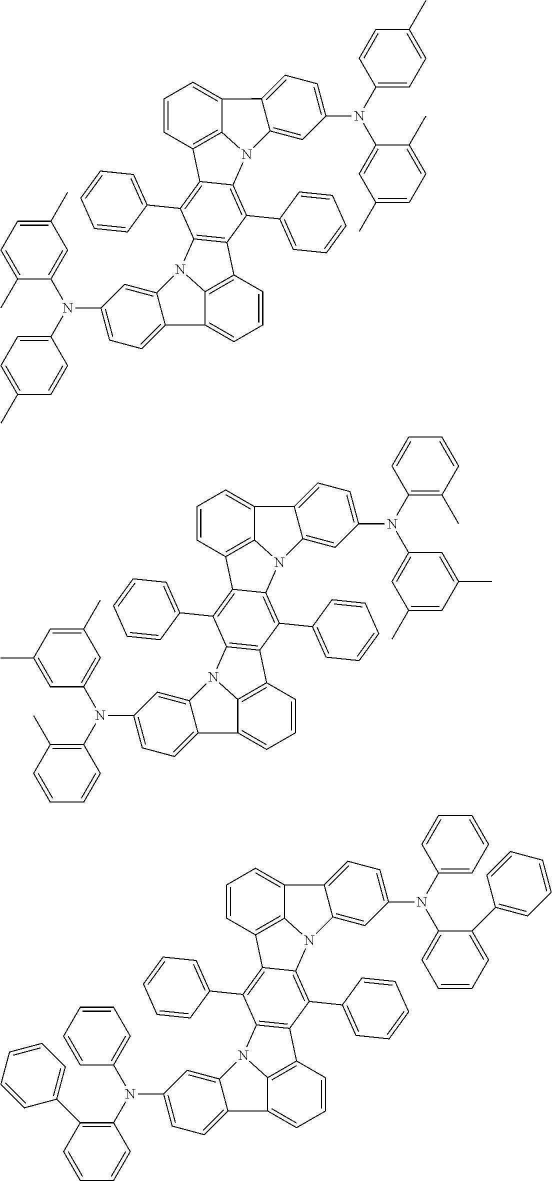

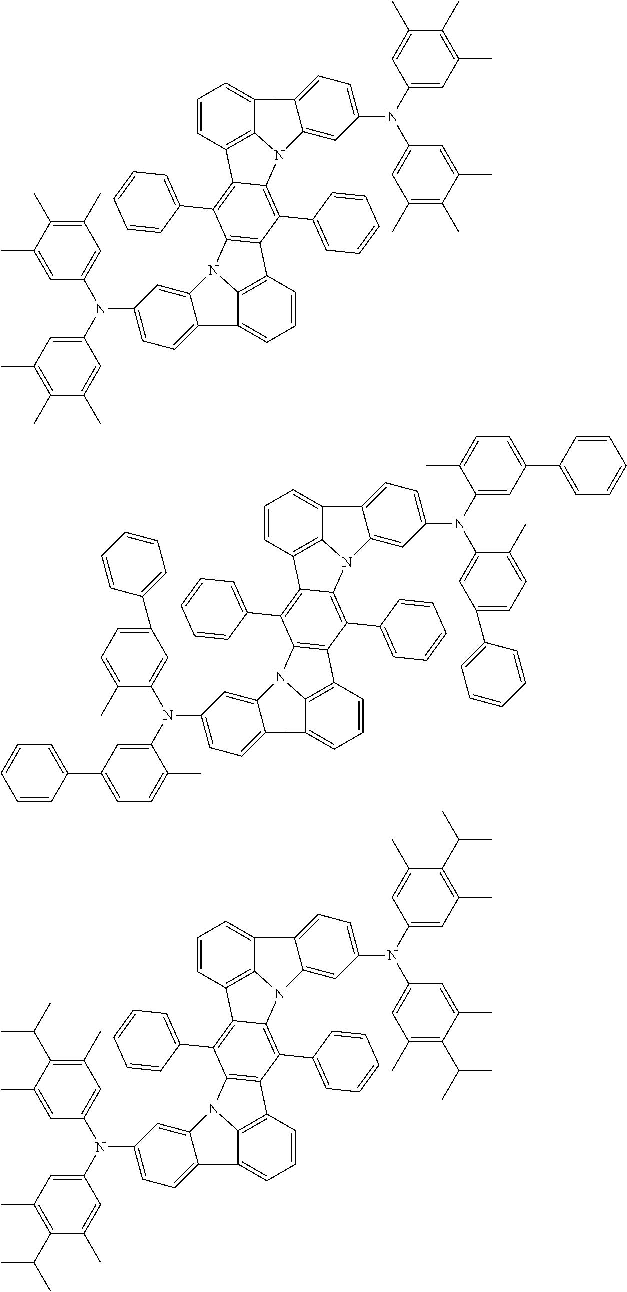

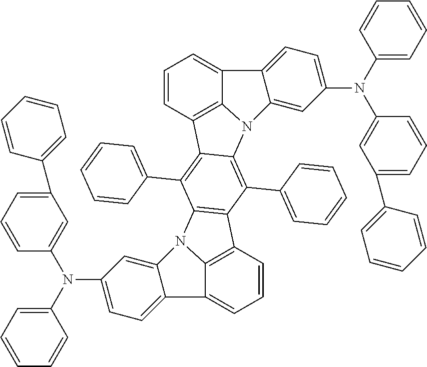 [SMILES: c1ccc(-c2cccc(N(c3ccccc3)c3ccc4c5cccc6c7c(-c8ccccc8)c8c(c(-c9ccccc9)c7n(c4c3)c56)c3cccc4c5ccc(N(c6ccccc6)c6cccc(-c7ccccc7)c6)cc5n8c43)c2)cc1]

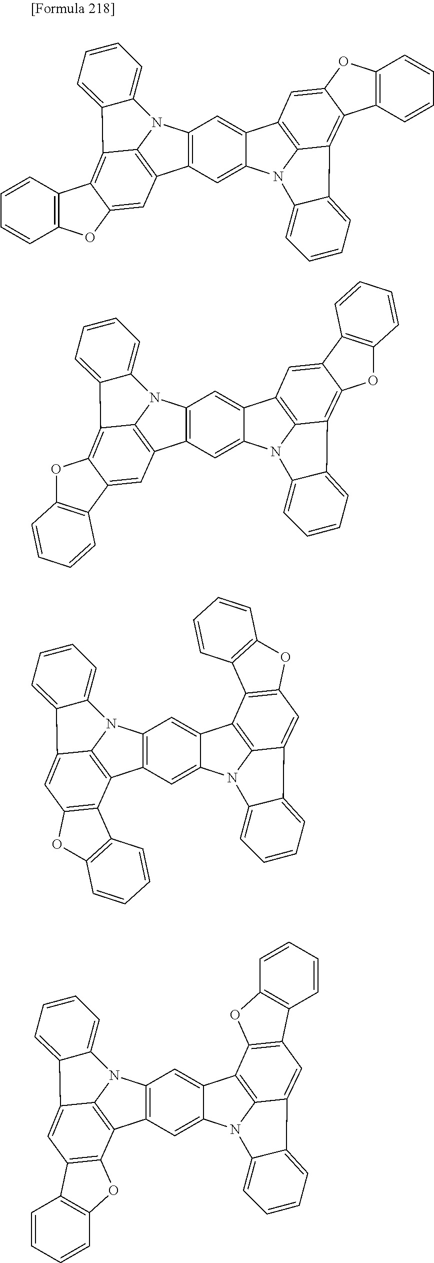

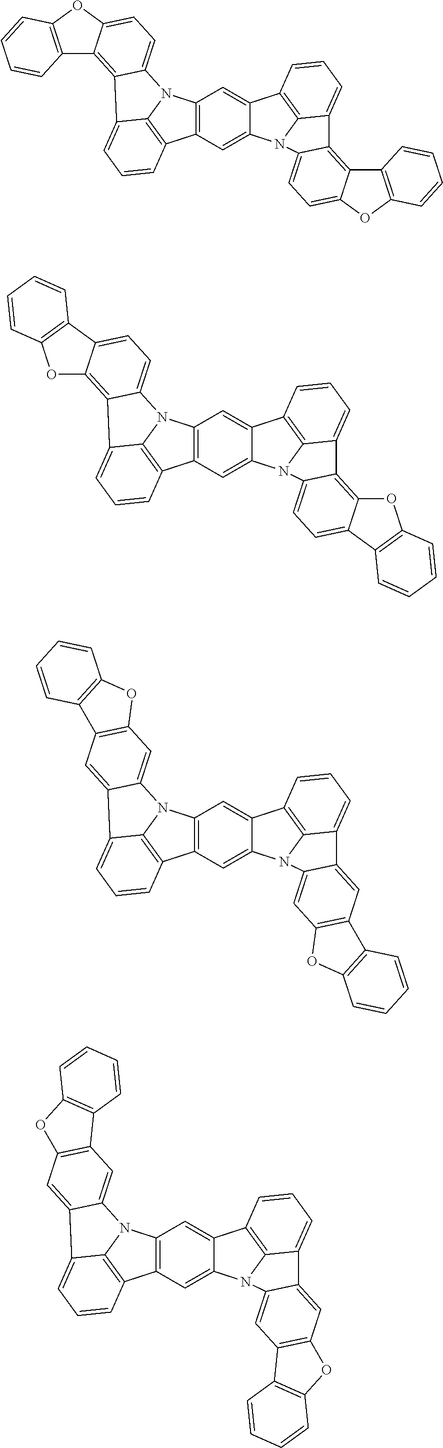 [SMILES: c1ccc2c(c1)oc1ccc3c(c4cccc5c6cc7c(cc6n3c54)c3cccc4c5c6c(ccc5n7c34)oc3ccccc36)c12 ; c1ccc2c(c1)oc1c2ccc2c1c1cccc3c4cc5c(cc4n2c31)c1cccc2c3c4oc6ccccc6c4ccc3n5c12 ; c1ccc2c(c1)oc1cc3c(cc12)c1cccc2c4cc5c(cc4n3c12)c1cccc2c3cc4c(cc3n5c21)oc1ccccc14 ; c1ccc2c(c1)oc1cc3c4cccc5c6cc7c(cc6n(c3cc12)c45)c1cccc2c3cc4oc5ccccc5c4cc3n7c21]

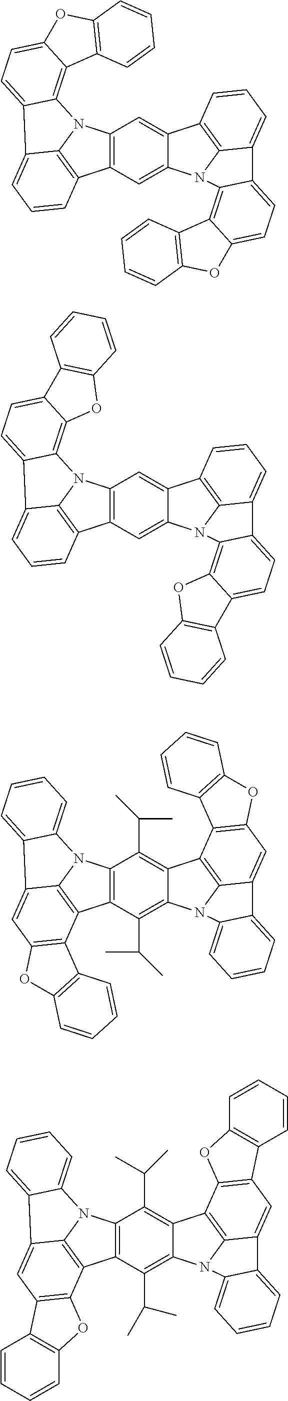 [SMILES: c1ccc2c(c1)oc1ccc3c4cccc5c6cc7c(cc6n(c54)c3c12)c1cccc2c3ccc4oc5ccccc5c4c3n7c12 ; c1ccc2c(c1)oc1c2ccc2c3cccc4c5cc6c(cc5n(c43)c21)c1cccc2c3ccc4c5ccccc5oc4c3n6c12 ; CC(C)c1c2c3c4c(cc5c6ccccc6n(c2c(C(C)C)c2c6c7c(cc8c9ccccc9n(c12)c86)oc1ccccc17)c53)oc1ccccc14 ; CC(C)c1c2c3c4oc5ccccc5c4cc4c5ccccc5n(c2c(C(C)C)c2c5c6oc7ccccc7c6cc6c7ccccc7n(c12)c65)c43]

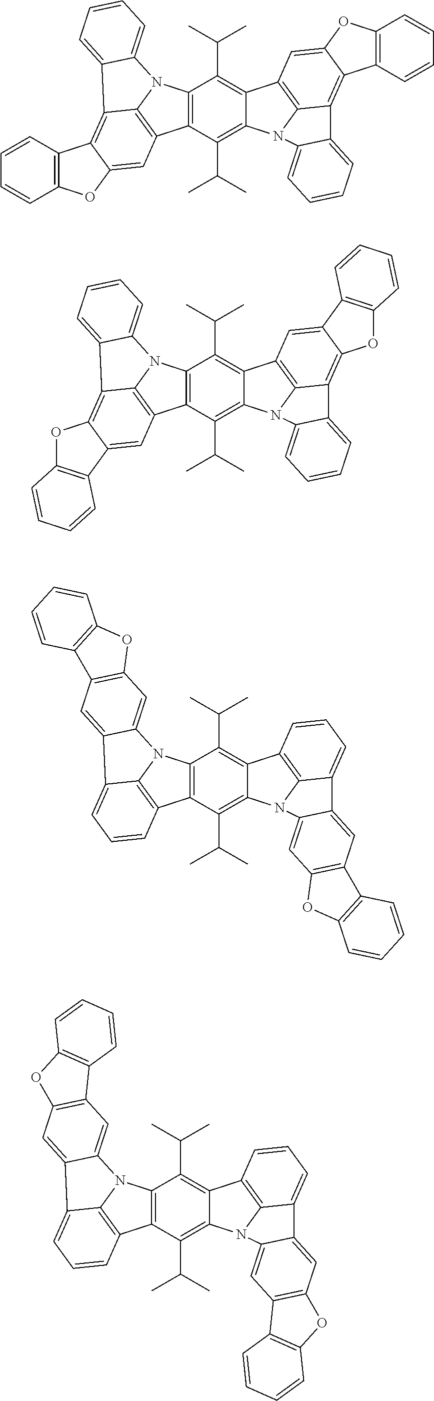 [SMILES: CC(C)c1c2c3cc4oc5ccccc5c4c4c5ccccc5n(c2c(C(C)C)c2c5cc6oc7ccccc7c6c6c7ccccc7n(c12)c56)c34 ; CC(C)c1c2c3cc4c5ccccc5oc4c4c5ccccc5n(c2c(C(C)C)c2c5cc6c7ccccc7oc6c6c7ccccc7n(c12)c56)c34 ; CC(C)c1c2c3cccc4c5cc6c(cc5n(c2c(C(C)C)c2c5cccc7c8cc9c(cc8n(c12)c75)oc1ccccc19)c43)oc1ccccc16 ; CC(C)c1c2c3cccc4c5cc6oc7ccccc7c6cc5n(c2c(C(C)C)c2c5cccc6c7cc8oc9ccccc9c8cc7n(c12)c65)c43]

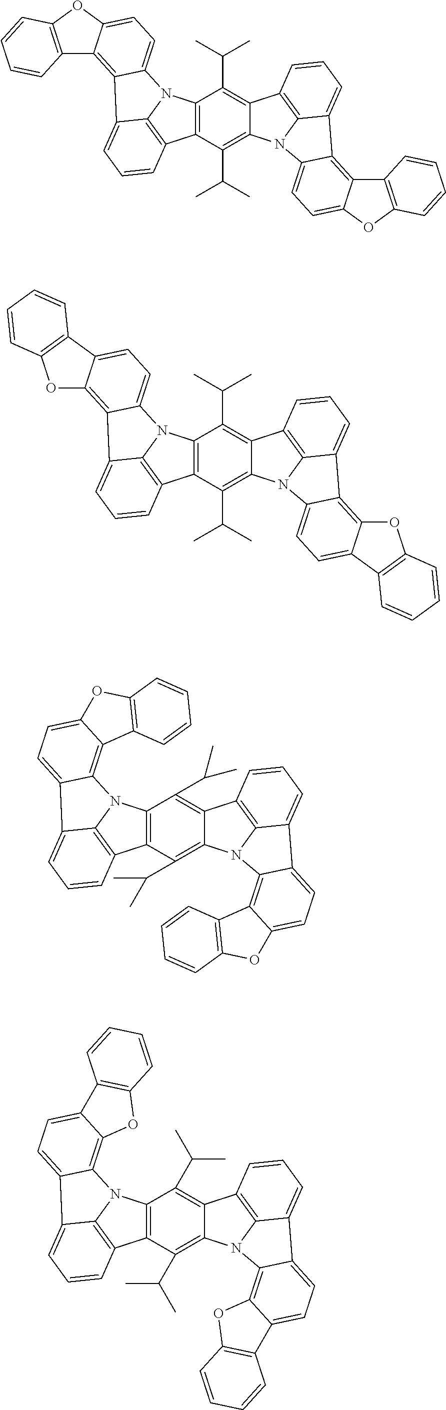 [SMILES: CC(C)c1c2c3cccc4c5c6c(ccc5n(c2c(C(C)C)c2c5cccc7c8c9c(ccc8n(c12)c57)oc1ccccc19)c34)oc1ccccc16 ; CC(C)c1c2c3cccc4c5c6oc7ccccc7c6ccc5n(c2c(C(C)C)c2c5cccc6c7c8oc9ccccc9c8ccc7n(c12)c56)c34 ; CC(C)c1c2c3cccc4c5ccc6oc7ccccc7c6c5n(c2c(C(C)C)c2c5cccc6c7ccc8oc9ccccc9c8c7n(c12)c65)c43 ; CC(C)c1c2c3cccc4c5ccc6c7ccccc7oc6c5n(c2c(C(C)C)c2c5cccc6c7ccc8c9ccccc9oc8c7n(c12)c65)c43]

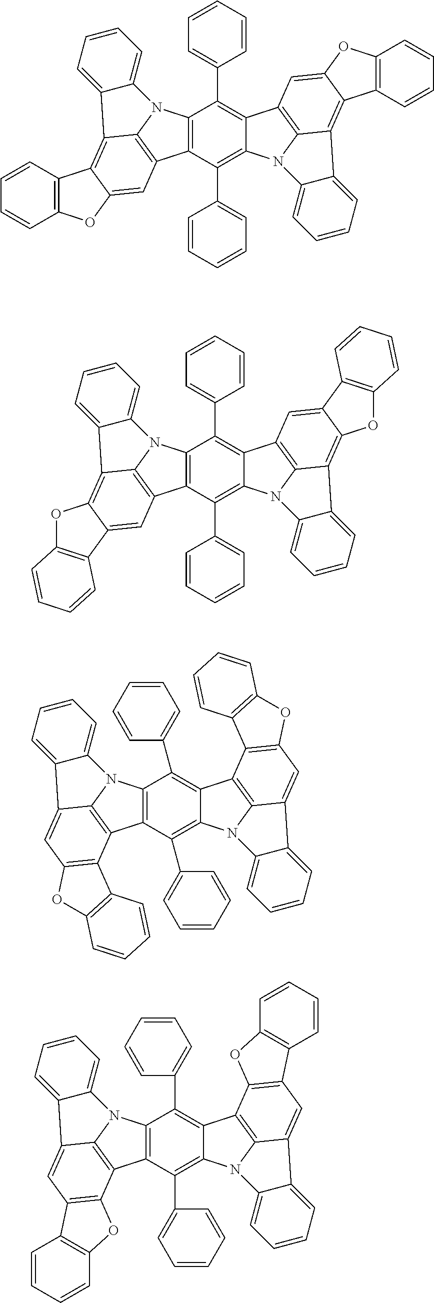 [SMILES: c1ccc(-c2c3c4cc5oc6ccccc6c5c5c6ccccc6n(c3c(-c3ccccc3)c3c6cc7oc8ccccc8c7c7c8ccccc8n(c23)c67)c45)cc1 ; c1ccc(-c2c3c4cc5c6ccccc6oc5c5c6ccccc6n(c3c(-c3ccccc3)c3c6cc7c8ccccc8oc7c7c8ccccc8n(c23)c67)c45)cc1 ; c1ccc(-c2c3c4c5c(cc6c7ccccc7n(c3c(-c3ccccc3)c3c7c8c(cc9c%10ccccc%10n(c23)c97)oc2ccccc28)c64)oc2ccccc25)cc1 ; c1ccc(-c2c3c4c5oc6ccccc6c5cc5c6ccccc6n(c3c(-c3ccccc3)c3c6c7oc8ccccc8c7cc7c8ccccc8n(c23)c76)c54)cc1]

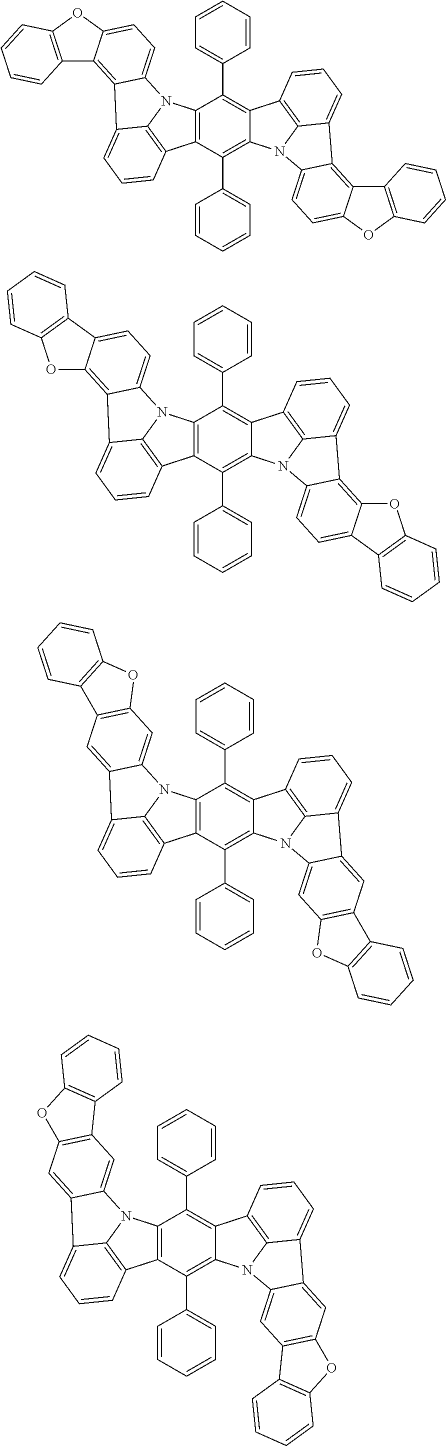 [SMILES: c1ccc(-c2c3c4cccc5c6c7c(ccc6n(c3c(-c3ccccc3)c3c6cccc8c9c%10c(ccc9n(c23)c68)oc2ccccc2%10)c45)oc2ccccc27)cc1 ; c1ccc(-c2c3c4cccc5c6c7oc8ccccc8c7ccc6n(c3c(-c3ccccc3)c3c6cccc7c8c9oc%10ccccc%10c9ccc8n(c23)c67)c45)cc1 ; c1ccc(-c2c3c4cccc5c6cc7c(cc6n(c3c(-c3ccccc3)c3c6cccc8c9cc%10c(cc9n(c23)c86)oc2ccccc2%10)c54)oc2ccccc27)cc1 ; c1ccc(-c2c3c4cccc5c6cc7oc8ccccc8c7cc6n(c3c(-c3ccccc3)c3c6cccc7c8cc9oc%10ccccc%10c9cc8n(c23)c76)c54)cc1]

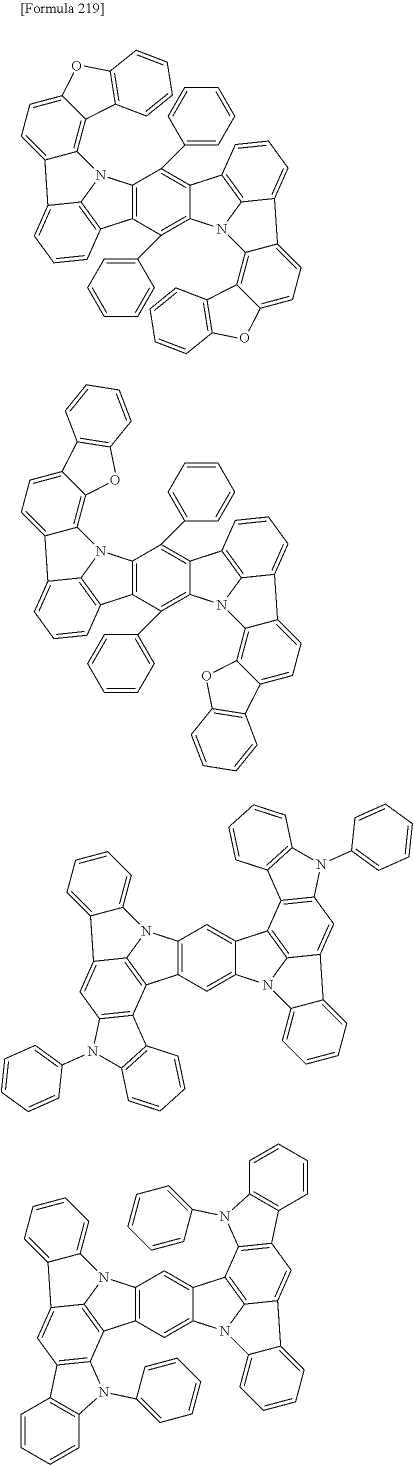

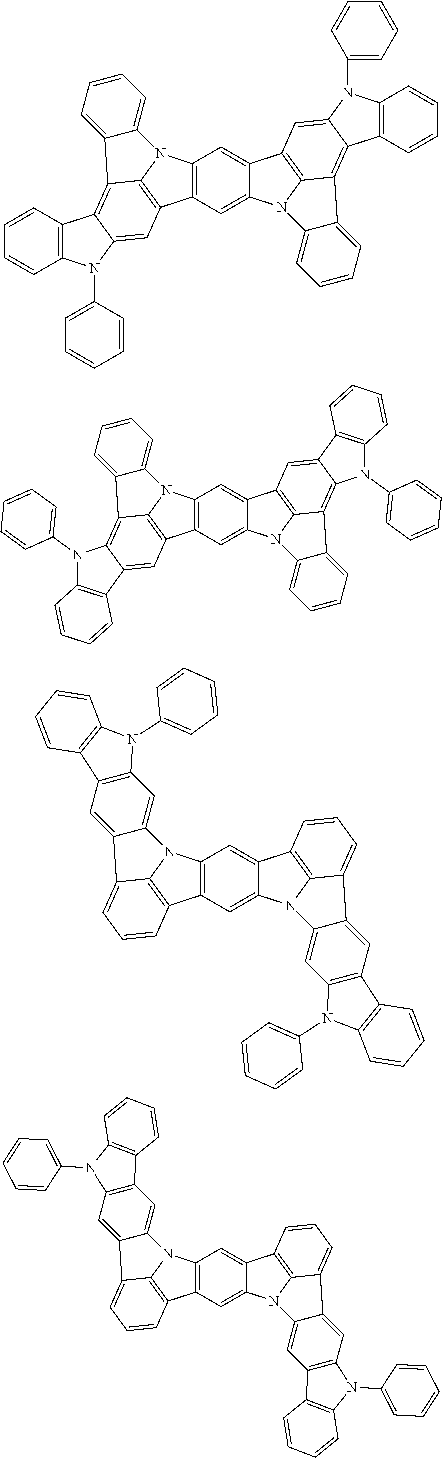 [SMILES: c1ccc(-n2c3ccccc3c3c4c5ccccc5n5c6cc7c8cc9c(c%10ccccc%10n9-c9ccccc9)c9c%10ccccc%10n(c7cc6c(cc32)c45)c89)cc1 ; c1ccc(-n2c3ccccc3c3cc4c5cc6c(cc5n5c7ccccc7c(c32)c45)c2cc3c4ccccc4n(-c4ccccc4)c3c3c4ccccc4n6c23)cc1 ; c1ccc(-n2c3ccccc3c3cc4c5cccc6c7cc8c(cc7n(c4cc32)c65)c2cccc3c4cc5c6ccccc6n(-c6ccccc6)c5cc4n8c23)cc1 ; c1ccc(-n2c3ccccc3c3cc4c(cc32)c2cccc3c5cc6c(cc5n4c23)c2cccc3c4cc5c(cc4n6c32)c2ccccc2n5-c2ccccc2)cc1]

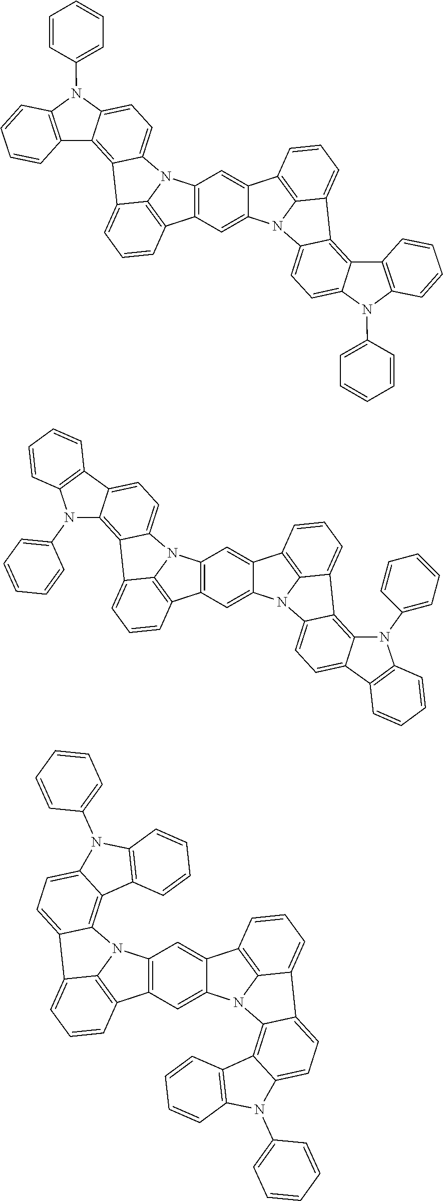 [SMILES: c1ccc(-n2c3ccccc3c3c4c5cccc6c7cc8c(cc7n(c4ccc32)c65)c2cccc3c4c5c6ccccc6n(-c6ccccc6)c5ccc4n8c23)cc1 ; c1ccc(-n2c3ccccc3c3ccc4c(c5cccc6c7cc8c(cc7n4c65)c4cccc5c6c7c(ccc6n8c45)c4ccccc4n7-c4ccccc4)c32)cc1 ; c1ccc(-n2c3ccccc3c3c2ccc2c4cccc5c6cc7c(cc6n(c54)c23)c2cccc3c4ccc5c(c6ccccc6n5-c5ccccc5)c4n7c23)cc1]

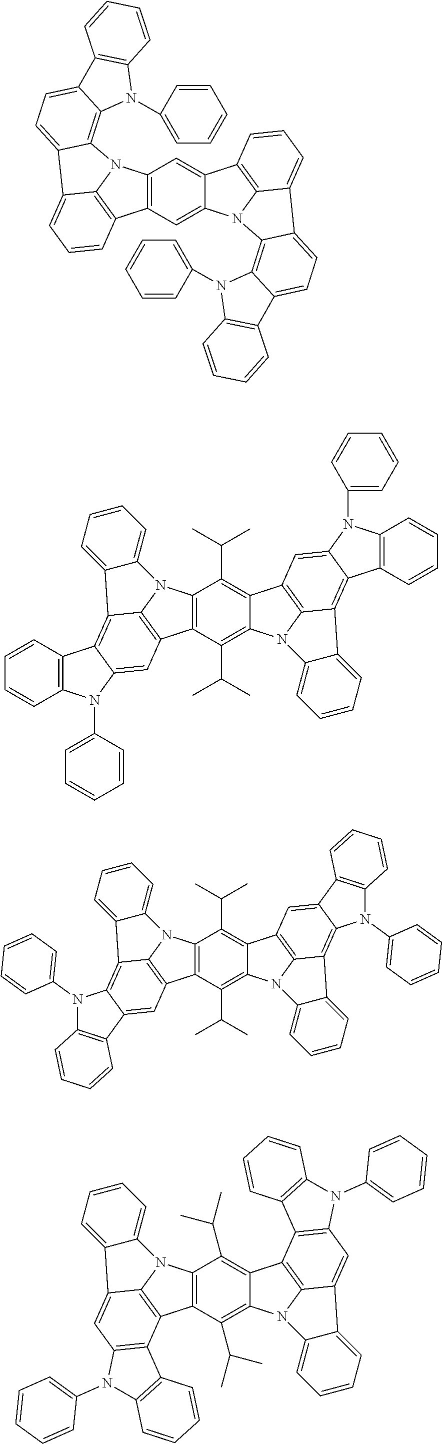 [SMILES: c1ccc(-n2c3ccccc3c3ccc4c5cccc6c7cc8c(cc7n(c65)c4c32)c2cccc3c4ccc5c6ccccc6n(-c6ccccc6)c5c4n8c23)cc1 ; CC(C)c1c2c3cc4c(c5ccccc5n4-c4ccccc4)c4c5ccccc5n(c2c(C(C)C)c2c5cc6c(c7ccccc7n6-c6ccccc6)c6c7ccccc7n(c12)c56)c34 ; CC(C)c1c2c3cc4c5ccccc5n(-c5ccccc5)c4c4c5ccccc5n(c2c(C(C)C)c2c5cc6c7ccccc7n(-c7ccccc7)c6c6c7ccccc7n(c12)c56)c34 ; CC(C)c1c2c3c4c5ccccc5n(-c5ccccc5)c4cc4c5ccccc5n(c2c(C(C)C)c2c5c6c7ccccc7n(-c7ccccc7)c6cc6c7ccccc7n(c12)c65)c43]

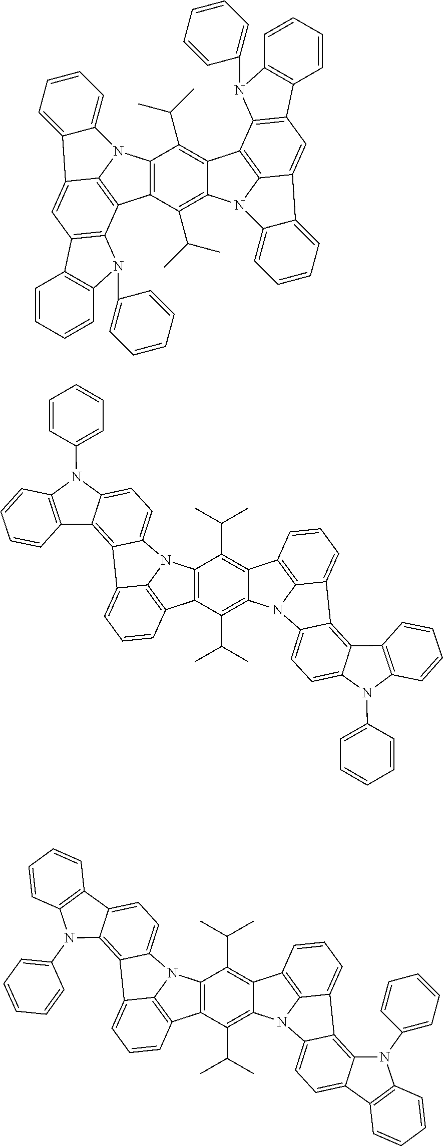 [SMILES: CC(C)c1c2c3c4c(cc5c6ccccc6n(c2c(C(C)C)c2c6c7c(cc8c9ccccc9n(c12)c86)c1ccccc1n7-c1ccccc1)c53)c1ccccc1n4-c1ccccc1 ; CC(C)c1c2c3cccc4c5c6c7ccccc7n(-c7ccccc7)c6ccc5n(c2c(C(C)C)c2c5cccc6c7c8c9ccccc9n(-c9ccccc9)c8ccc7n(c12)c56)c34 ; CC(C)c1c2c3cccc4c5c6c(ccc5n(c2c(C(C)C)c2c5cccc7c8c9c(ccc8n(c12)c57)c1ccccc1n9-c1ccccc1)c34)c1ccccc1n6-c1ccccc1]

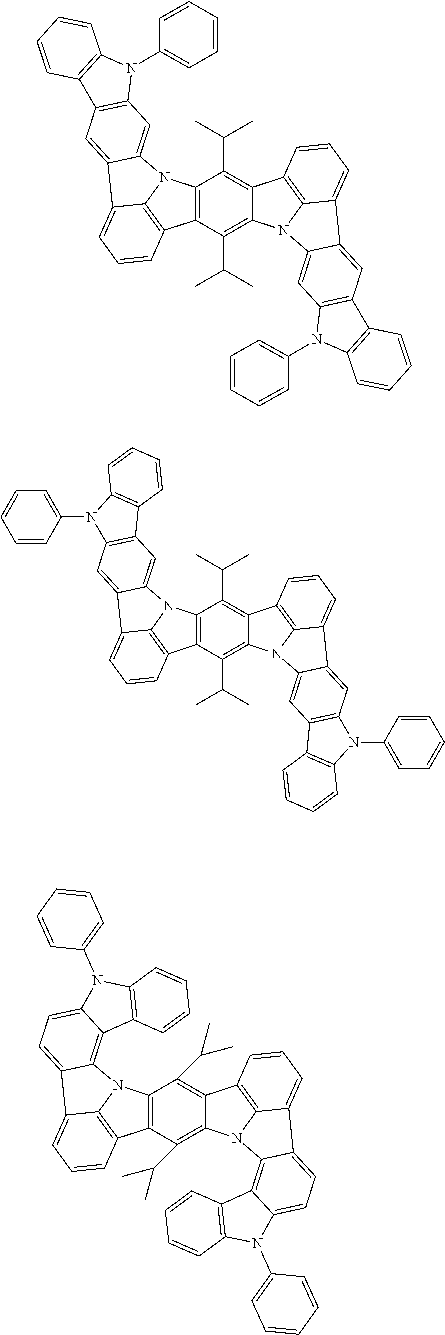 [SMILES: CC(C)c1c2c3cccc4c5cc6c7ccccc7n(-c7ccccc7)c6cc5n(c2c(C(C)C)c2c5cccc6c7cc8c9ccccc9n(-c9ccccc9)c8cc7n(c12)c65)c43 ; CC(C)c1c2c3cccc4c5cc6c(cc5n(c2c(C(C)C)c2c5cccc7c8cc9c(cc8n(c12)c75)c1ccccc1n9-c1ccccc1)c43)c1ccccc1n6-c1ccccc1 ; CC(C)c1c2c3cccc4c5ccc6c(c7ccccc7n6-c6ccccc6)c5n(c2c(C(C)C)c2c5cccc6c7ccc8c(c9ccccc9n8-c8ccccc8)c7n(c12)c65)c43]

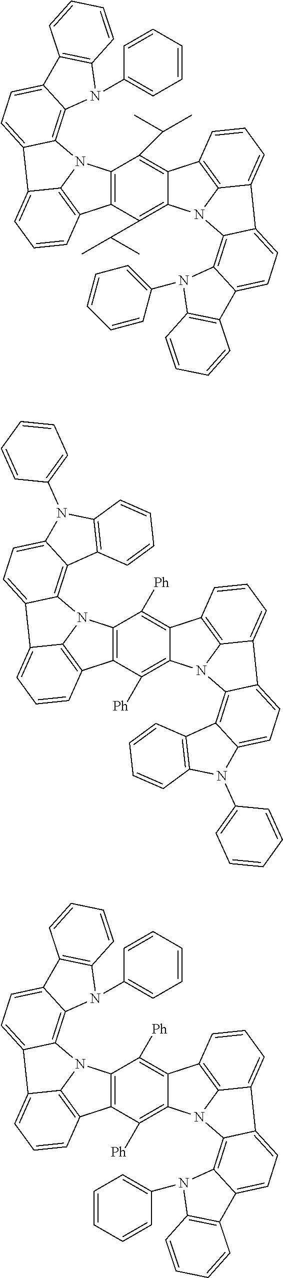 [SMILES: CC(C)c1c2c3cccc4c5ccc6c7ccccc7n(-c7ccccc7)c6c5n(c2c(C(C)C)c2c5cccc6c7ccc8c9ccccc9n(-c9ccccc9)c8c7n(c12)c65)c43 ; c1ccc(-c2c3c4cccc5c6ccc7c(c8ccccc8n7-c7ccccc7)c6n(c3c(-c3ccccc3)c3c6cccc7c8ccc9c(c%10ccccc%10n9-c9ccccc9)c8n(c23)c76)c54)cc1 ; c1ccc(-c2c3c4cccc5c6ccc7c8ccccc8n(-c8ccccc8)c7c6n(c3c(-c3ccccc3)c3c6cccc7c8ccc9c%10ccccc%10n(-c%10ccccc%10)c9c8n(c23)c76)c54)cc1]

(In the formulae, Ph denotes a phenyl group.)

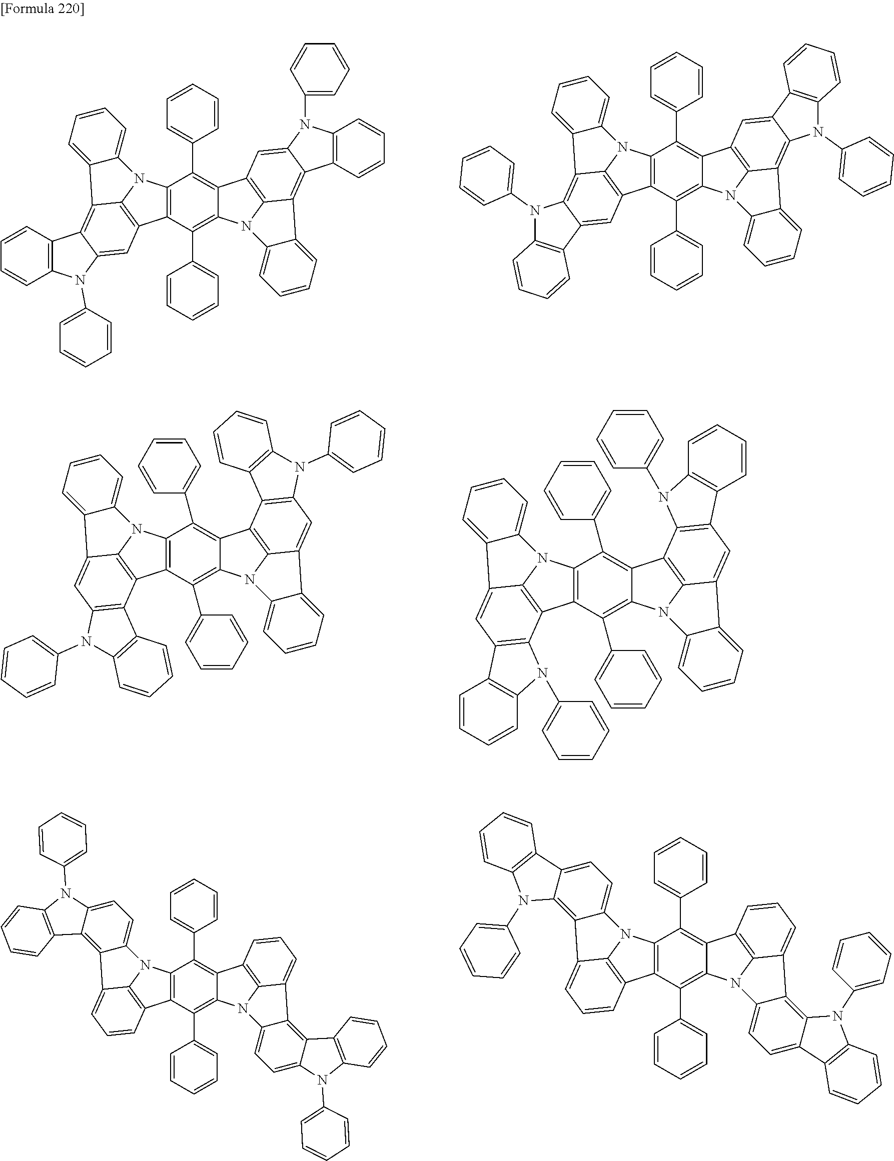

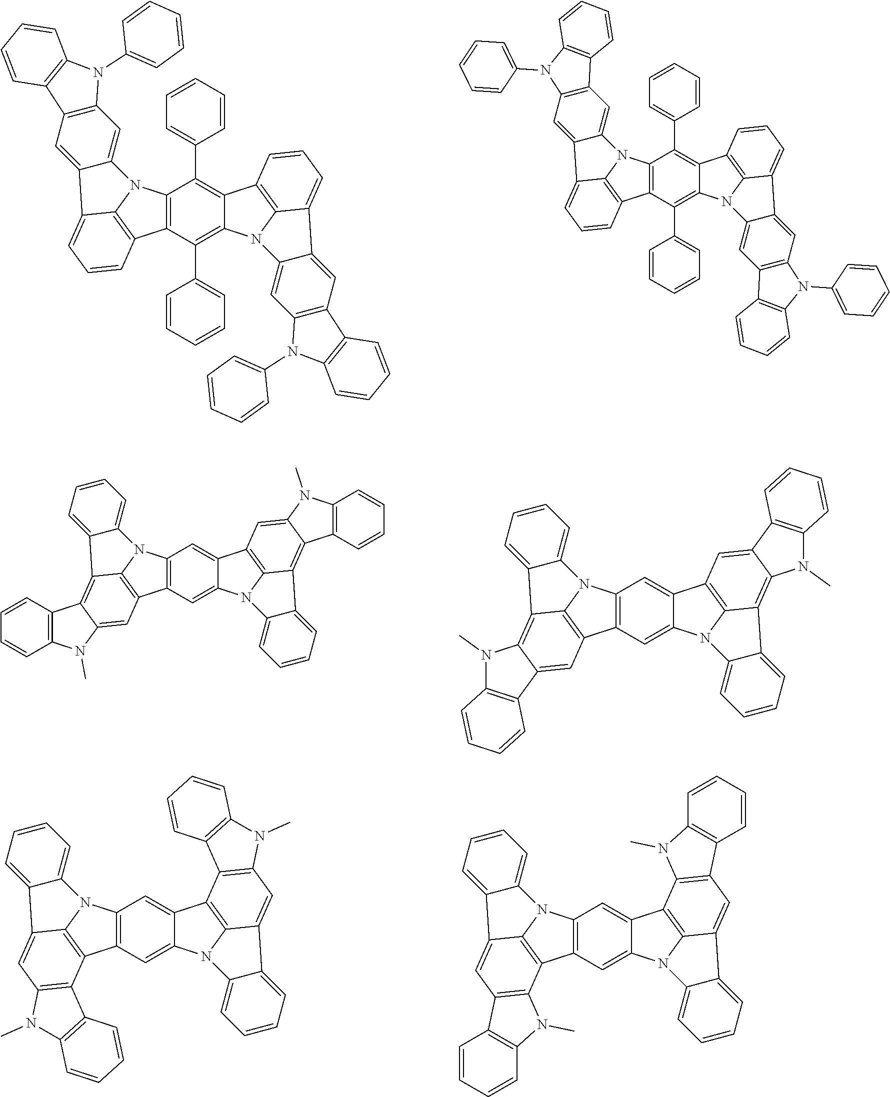

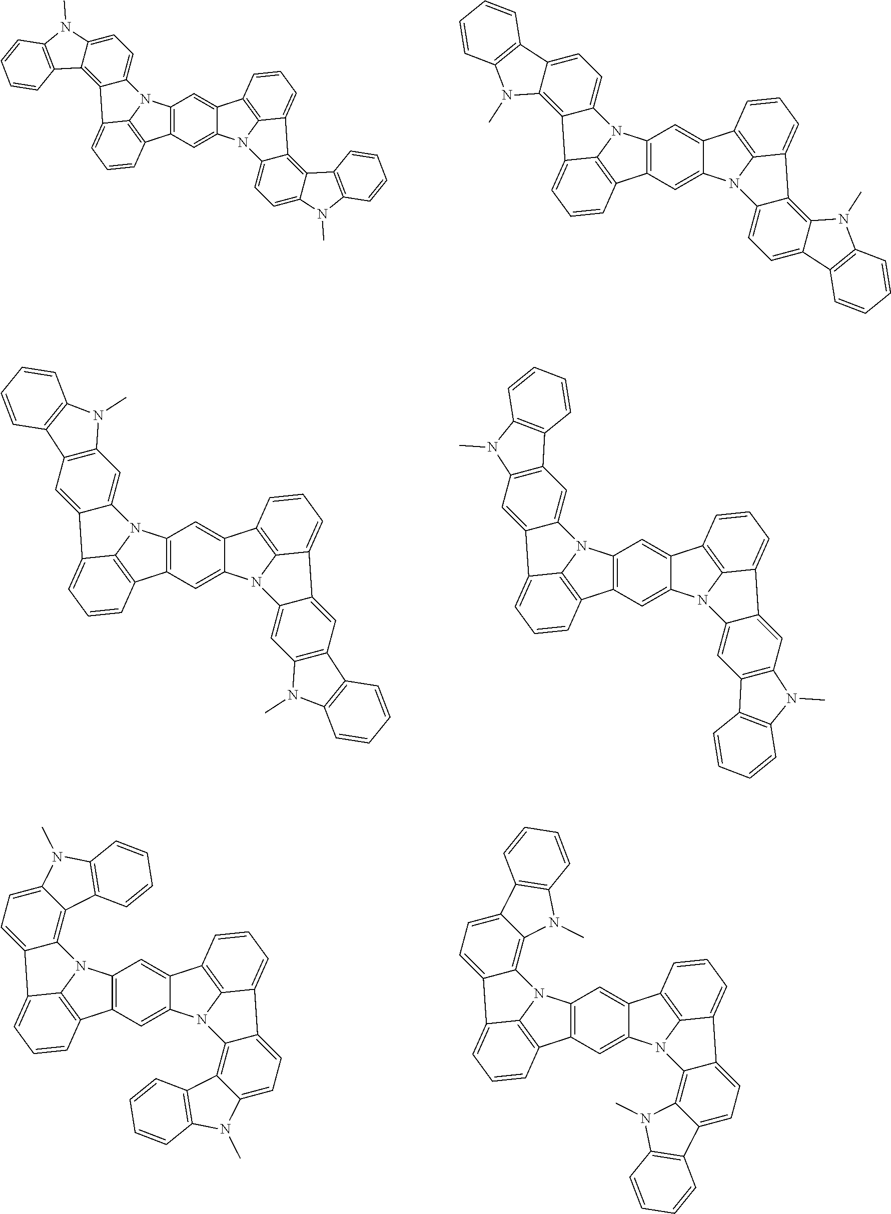

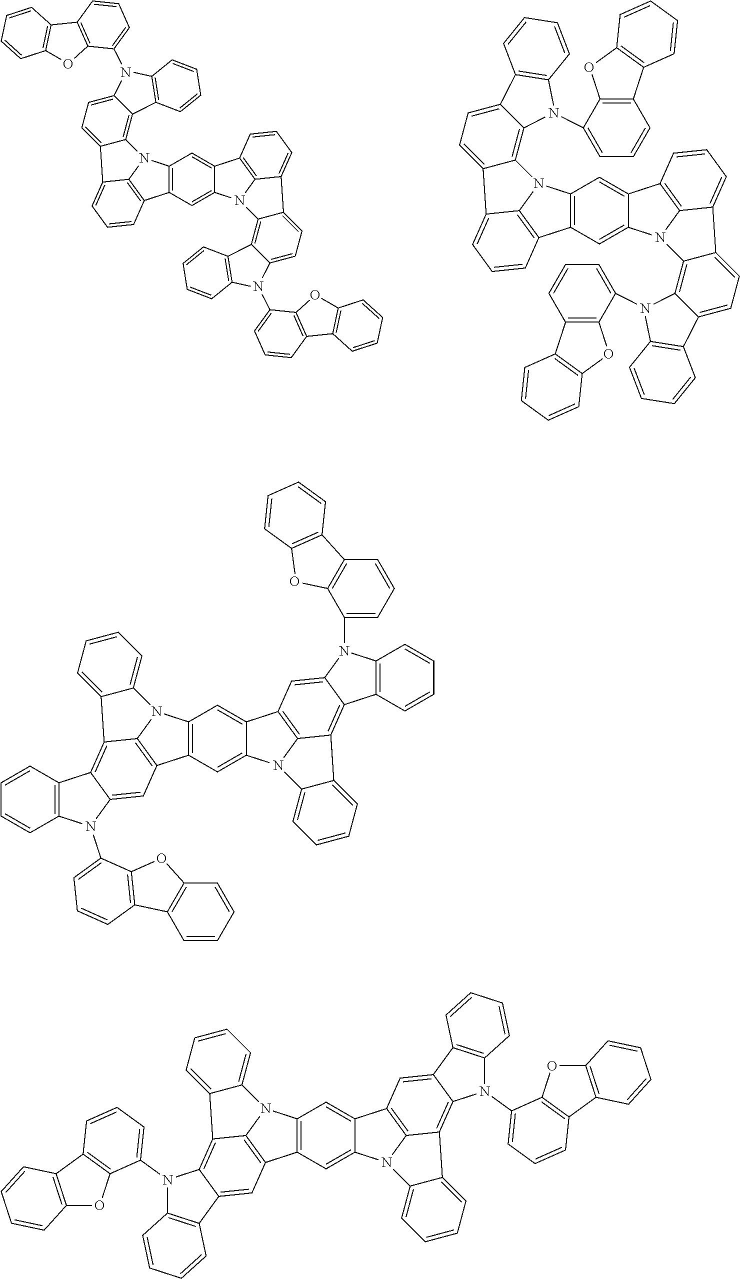

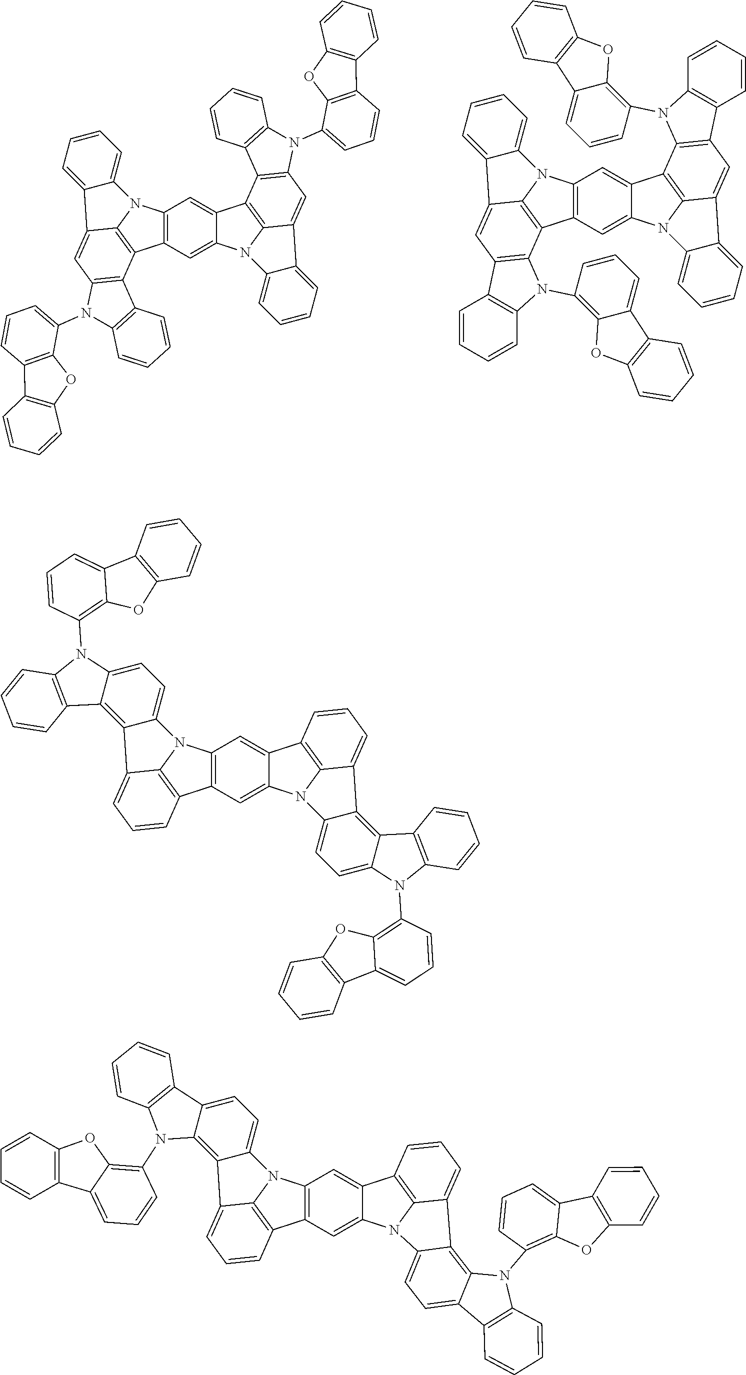

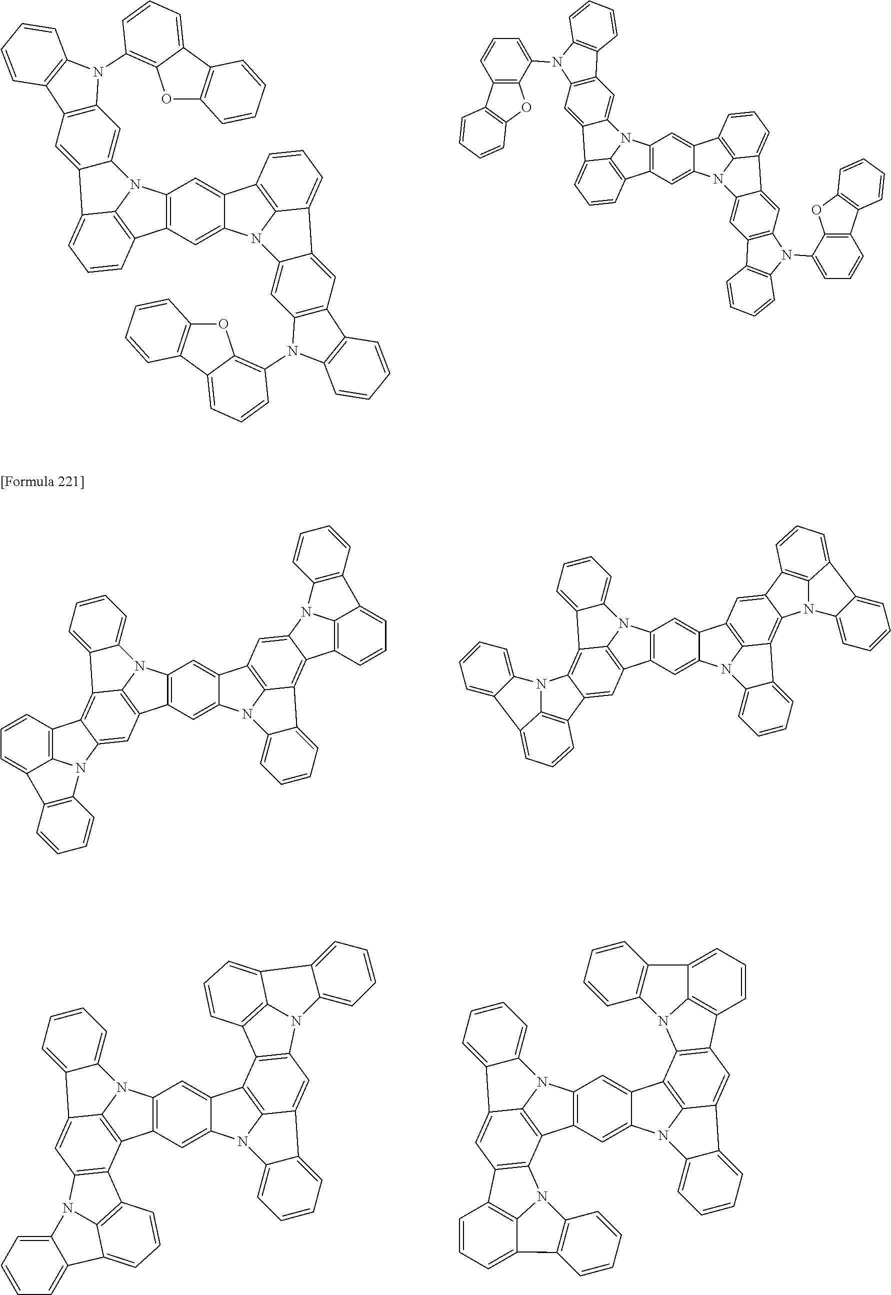

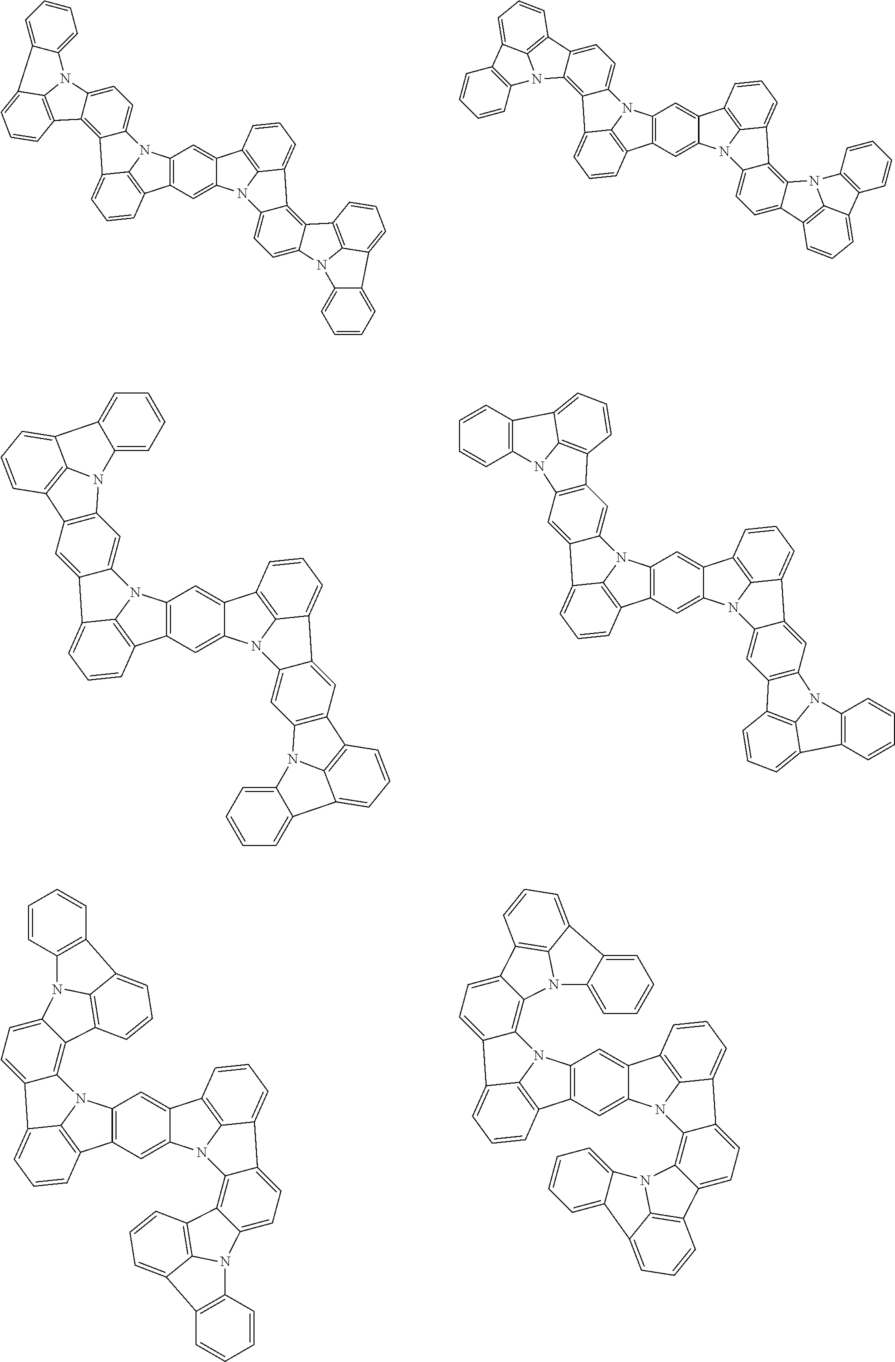

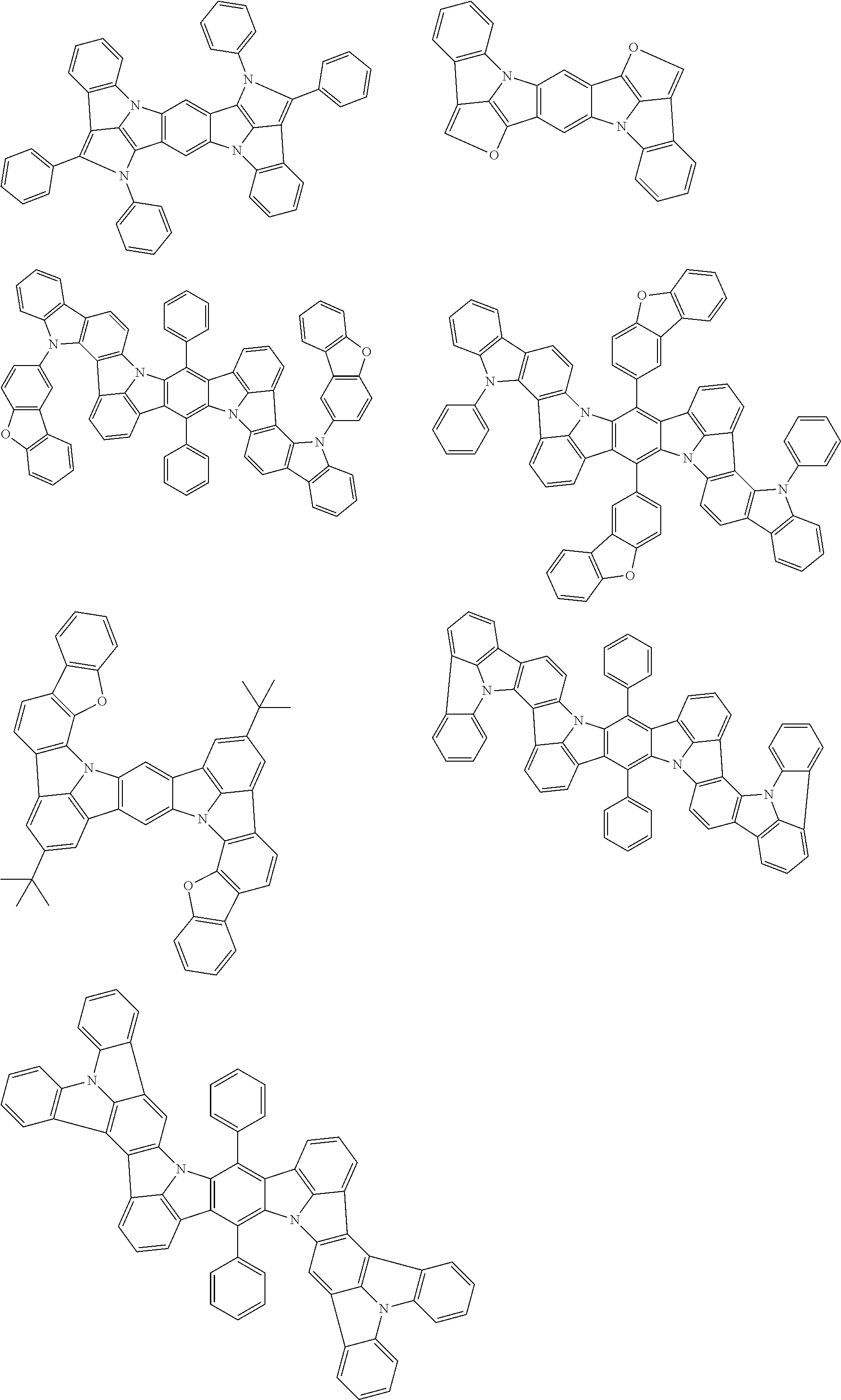

Compound Represented by Formula (6)

A compound represented by the formula (6) is described below.

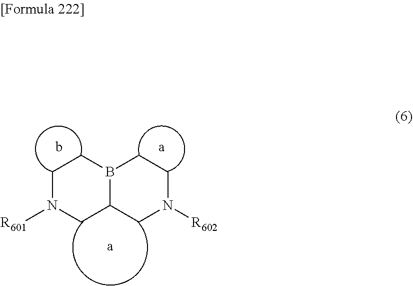

In the formula (6),
the a ring, the b ring, and the c ring are each independently a substituted or unsubstituted aromatic hydrocarbon ring having 6 to 50 ring carbon atoms, or a substituted or unsubstituted heterocycle having 5 to 50 ring atoms,
R₆₀₁ and R₆₀₂ are each independently bonded to the a ring, the b ring, or the c ring to form a substituted or unsubstituted heterocycle, or do not form a substituted or unsubstituted heterocycle, and
R₆₀₁ and R₆₀₂ that do not form the substituted or unsubstituted heterocycle each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The a ring, the b ring, and the c ring are rings (substituted or unsubstituted aromatic hydrocarbon rings having 6 to 50 ring carbon atoms or substituted or unsubstituted heterocycles having 5 to 50 ring atoms) fused to the central fused bicyclic structure of the formula (6) including a boron atom and two nitrogen atoms.

The “aromatic hydrocarbon rings” of the a ring, the b ring, and the c ring have the same structure as compounds in which a hydrogen atom is introduced into the “aryl group” described above.

The “aromatic hydrocarbon ring” of the a ring has three carbon atoms on the central fused bicyclic structure of the formula (6) as ring atoms.

The “aromatic hydrocarbon rings” of the b and c rings have two carbon atoms on the central fused bicyclic structure of the formula (6) as ring atoms.

Specific examples of the “substituted or unsubstituted aromatic hydrocarbon ring having 6 to 50 ring carbon atoms” include compounds in which a hydrogen atom is introduced into the “aryl group” described in the specific example group G1.

The “heterocycles” of the a ring, the b ring, and the c ring have the same structure as compounds in which a hydrogen atom is introduced into the “heterocyclic group” described above.

The “heterocycle” of the a ring has three carbon atoms on the central fused bicyclic structure of the formula (6) as ring atoms. The “heterocycles” of the b ring and the c ring have two carbon atoms on the central fused bicyclic structure of the formula (6) as ring atoms. Specific examples of the “substituted or unsubstituted heterocycle having 5 to 50 ring atoms” include compounds in which a hydrogen atom is introduced into the “heterocyclic group” described in the specific example group G2.

R₆₀₁ and R₆₀₂ may be each independently bonded to the a ring, the b ring, or the c ring to form a substituted or unsubstituted heterocycle. This heterocycle has the nitrogen atom on the central fused bicyclic structure of the formula (6). The heterocycle may have a heteroatom other than the nitrogen atom. The phrase “R₆₀₁ and R₆₀₂ are each independently bonded to the a ring, the b ring, or the c ring” more specifically means that an atom constituting the a ring, the b ring, or the c ring is bonded to an atom constituting R₆₀₁ and R₆₀₂. For example, R₆₀₁ may be bonded to the a ring to form a fused bicyclic (or fused tricyclic or higher cyclic) nitrogen-containing heterocycle in which the ring with R₆₀₁ is fused to the a ring. Specific examples of the nitrogen-containing heterocycle include compounds corresponding to fused bicyclic or higher cyclic heterocyclic group containing nitrogen of the specific example group G2.

The same applies to when R₆₀₁ is bonded to the b ring, when R₆₀₂ is bonded to the a ring, and when R₆₀₂ is bonded to the c ring.

In an exemplary embodiment, the a ring, the b ring, and the c ring in the formula (6) are each independently a substituted or unsubstituted aromatic hydrocarbon ring having 6 to 50 ring carbon atoms.

In an exemplary embodiment, the a ring, the b ring, and the c ring in the formula (6) are each independently a substituted or unsubstituted benzene ring or naphthalene ring.

In an exemplary embodiment, R₆₀₁ and R₆₀₂ in the formula (6) each independently represent a substituted or unsubstituted aryl group having 6 to 50 ring carbon atoms, or a substituted or unsubstituted heterocyclic group having 5 to 50 ring atoms, preferably a substituted or unsubstituted aryl group having 6 to 50 ring carbon atoms.

In an exemplary embodiment, a compound represented by the formula (6) is a compound represented by the following formula (62).

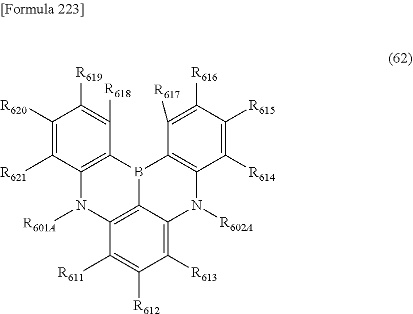

In the formula (62),
R_{601A} is bonded to at least one selected from the group consisting of R₆₁₁ and R₆₂₁ to form a substituted or unsubstituted heterocycle, or does not form a substituted or unsubstituted heterocycle,
R_{602A} is bonded to at least one selected from the group consisting of R₆₁₃ and R₆₁₄ to form a substituted or unsubstituted heterocycle, or does not form a substituted or unsubstituted heterocycle,
R_{601A} and R_{602A} that do not form the substituted or unsubstituted heterocycle each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,
at least one combination of adjacent two or more of R₆₁₁ to R₆₂₁ are mutually bonded to form a substituted or unsubstituted monocyclic ring, are mutually bonded to form a substituted or unsubstituted fused ring, or are not mutually bonded, and
R₆₁₁ to R₆₂₁ that do not form the substituted or unsubstituted heterocycle, do not form the monocyclic ring, and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.
R_{601A} and R_{602A} in the formula (62) are groups corresponding to R₆₀₁ and R₆₀₂, respectively, in the formula (6).

For example, R_{601A} may be bonded to R₆₁₁ to form a fused bicyclic (or fused tricyclic or higher cyclic) nitrogen-containing heterocycle in which and the ring with R_{601A} and R₆₁₁ is fused to a benzene ring corresponding to the a ring. Specific examples of the nitrogen-containing heterocycle include compounds corresponding to fused bicyclic or higher cyclic heterocyclic groups containing nitrogen of the specific example group G2. The same applies to when R_{601A} is bonded to R₆₂₁, when R_{602A} is bonded to R₆₁₃, and when R_{602A} is bonded to R₆₁₄.

At least one combination of adjacent two or more of R₆₁₁ to R₆₂₁
may be mutually bonded to form a substituted or unsubstituted monocyclic ring, or may be mutually bonded to form a substituted or unsubstituted fused ring.

For example, R₆₁₁ and R₆₁₂ may be mutually bonded to form a structure in which a benzene ring, an indole ring, a pyrrole ring, a benzofuran ring, or a benzothiophene ring is fused to the 6-membered ring bonded to R₆₁₁ and R₆₁₂. The fused ring thus formed is a naphthalene ring, a carbazole ring, an indole ring, a dibenzofuran ring, or a dibenzothiophene ring.

In an exemplary embodiment, R₆₁₁ to R₆₂₁ that do not contribute to ring formation each independently represent a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ that do not contribute to ring formation each independently represent a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ that do not contribute to ring formation each independently represent a hydrogen atom, or a substituted or unsubstituted alkyl group having 1 to 50 carbon atoms.

In an exemplary embodiment, R₆₁₁ to R₆₂₁ that do not contribute to ring formation each independently represent a hydrogen atom, or a substituted or unsubstituted alkyl group having 1 to 50 carbon atoms, in which at least one of R₆₁₁ to R₆₂₁ is a substituted or unsubstituted alkyl group having 1 to 50 carbon atoms.

In an exemplary embodiment, a compound represented by the formula (62) is a compound represented by the following formula (63).

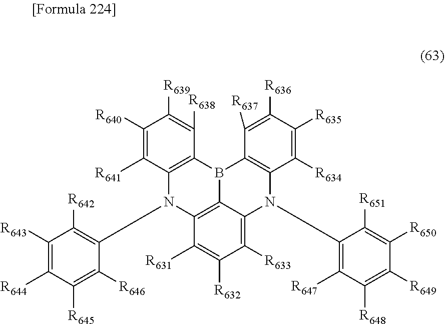

In the formula (63),
R₆₃₁ is bonded to R₆₄₆ to form a substituted or unsubstituted heterocycle, or does not form a substituted or unsubstituted heterocycle,
R₆₃₃ is bonded to R₆₄₇ to form a substituted or unsubstituted heterocycle, or does not form a substituted or unsubstituted heterocycle,
R₆₃₄ is bonded to R₆₅₁ to form a substituted or unsubstituted heterocycle, or does not form a substituted or unsubstituted heterocycle,
R₆₄₁ is bonded to R₆₄₂ to form a substituted or unsubstituted heterocycle, or does not form a substituted or unsubstituted heterocycle,
at least one combination of adjacent two or more of R₆₃₁ to R₆₅₁ are mutually bonded to form a substituted or unsubstituted monocyclic ring, are mutually bonded to form a substituted or unsubstituted fused ring, or are not mutually bonded, and
R₆₃₁ to R₆₅₁ that do not form the substituted or unsubstituted heterocycle, do not form the monocyclic ring, and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.
R₆₃₁ may be bonded to R₆₄₆ to form a substituted or unsubstituted heterocycle. For example, R₆₃₁ may be bonded to R₆₄₆ to form a fused tricyclic or higher cyclic nitrogen-containing heterocycle in which the benzene ring bonded to R₆₄₆, the ring containing N, and the benzene ring corresponding to the a ring are fused. Specific examples of the nitrogen-containing heterocycle include compounds corresponding to fused tricyclic or higher cyclic heterocyclic groups containing nitrogen of the specific example group G2. The same applies to when R₆₃₃ is bonded to R₆₄₇, when R₆₃₄ is bonded to R₆₅₁, and when R₆₄₁ is bonded to R₆₄₂.

In an exemplary embodiment, R₆₃₁ to R₆₅₁ that do not contribute to ring formation each independently represent a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ that do not contribute to ring formation each independently represent a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ that do not contribute to ring formation each independently represent a hydrogen atom, or a substituted or unsubstituted alkyl group having 1 to 50 carbon atoms.

In an exemplary embodiment, R₆₃₁ to R₆₅₁ that do not contribute to ring formation each independently represent a hydrogen atom, or a substituted or unsubstituted alkyl group having 1 to 50 carbon atoms, and at least one of R₆₃₁ to R₆₅₁ is a substituted or unsubstituted alkyl group having 1 to 50 carbon atoms.

In an exemplary embodiment, a compound represented by the formula (63) is a compound represented by the following formula (63A).

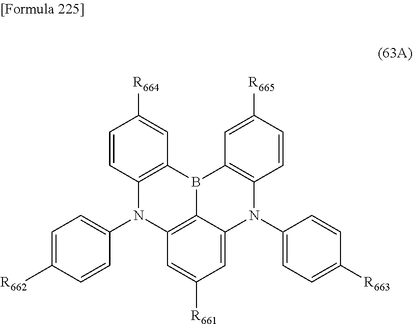

In the formula (63A),
R₆₆₁ represents a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms, and
R₆₆₂ to R₆₆₅ each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅ each independently represent a substituted or unsubstituted alkyl group having 1 to 50 carbon atoms, or a substituted or unsubstituted aryl group having 6 to 50 ring carbon atoms.

In an exemplary embodiment, R₆₆₁ to R₆₆₅ each independently represent a substituted or unsubstituted alkyl group having 1 to 50 carbon atoms.

In an exemplary embodiment, a compound represented by the formula (63) is a compound represented by the following formula (63B).

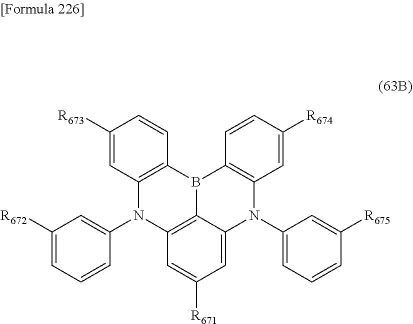

In the formula (63B),
R₆₇₁ and R₆₇₂ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms, and
R₆₇₃ to R₆₇₅ each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, a compound represented by the formula (63) is a compound represented by the following formula (63B′).

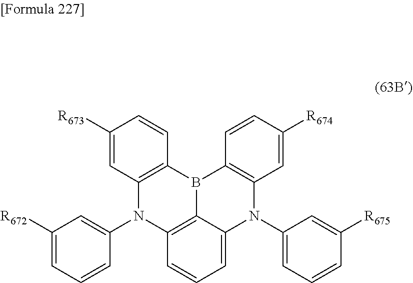

In the formula (63B′), R₆₇₂ to R₆₇₅ represent the same as R₆₇₂ to R₆₇₅, respectively, in the formula (63B).

In an exemplary embodiment, at least one of R₆₇₁ to R₆₇₅ represents a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, R₆₇₂ represents a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms, and
R₆₇₁ and R₆₇₃ to R₆₇₅ each independently represent a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, a compound represented by the formula (63) is a compound represented by the following formula (63C).

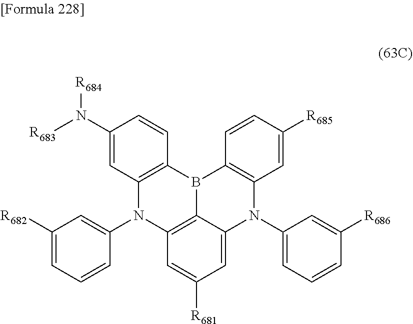

In the formula (63C),
R₆₈₁ and R₆₈₂ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

R₆₈₃ to R₆₈₆ each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, a compound represented by the formula (63) is a compound represented by the following formula (63C′).

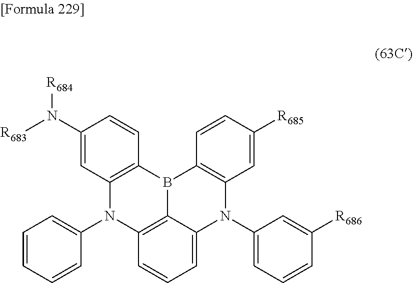

In the formula (63C′), R₆₈₃ to R₆₈₆ represent the same as R₆₈₃ to R₆₈₆, respectively, in the formula (63C).

In an exemplary embodiment, R₆₈₁ to R₆₈₆ each independently represent a substituted or unsubstituted alkyl group having 1 to 50 carbon atoms, or a substituted or unsubstituted aryl group having 6 to 50 ring carbon atoms.

In an exemplary embodiment, R₆₈₁ to R₆₈₆ each independently represent a substituted or unsubstituted aryl group having 6 to 50 ring carbon atoms.

A compound represented by the formula (6) can be produced by first bonding the a ring, the b ring, and the c ring to each other with a linking group (a group containing N—R₆₀₁ and a group containing N—R₆₀₂) to produce an intermediate (a first reaction) and then bonding the a ring, the b ring, and the c ring to each other with a linking group (a group containing a boron atom) to produce an end product (a second reaction). In the first reaction, an amination reaction, such as a Buchwald-Hartwig reaction, can be used. In the second reaction, a tandem hetero Friedel-Crafts reaction or the like can be used.

Specific examples of a compound represented by the formula (6) are described below. These are only examples, and a compound represented by the formula (6) is not limited to these specific examples.

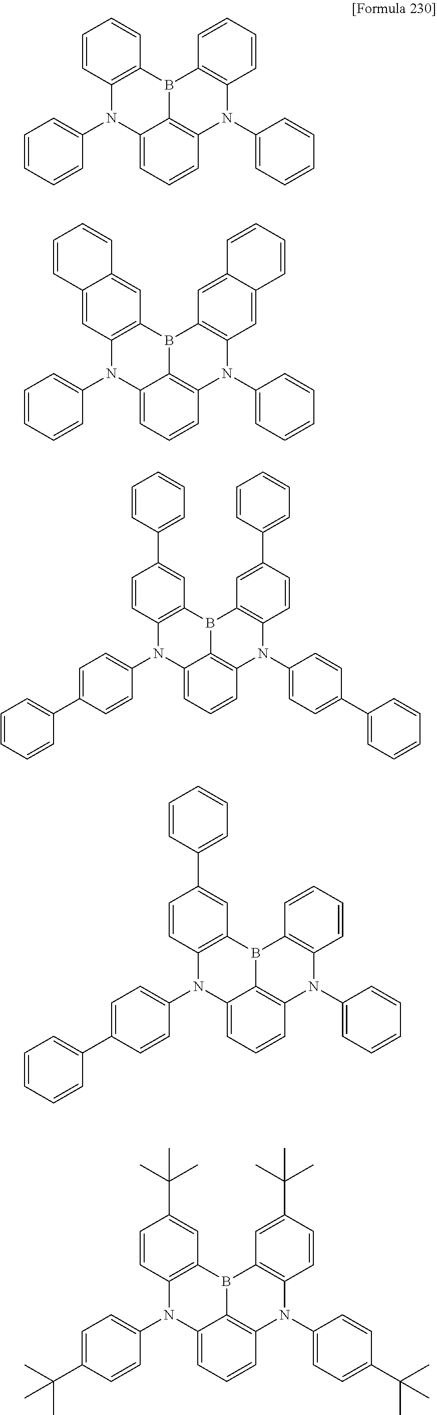

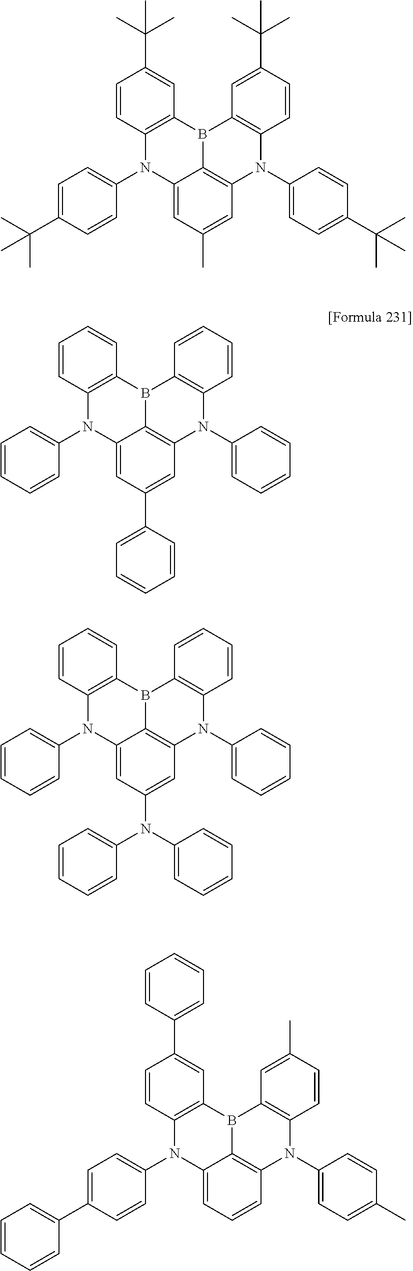 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(-c2cc3c4c(c2)N(c2ccccc2)c2ccccc2B4c2ccccc2N3c2ccccc2)cc1 ; c1ccc(N(c2ccccc2)c2cc3c4c(c2)N(c2ccccc2)c2ccccc2B4c2ccccc2N3c2ccccc2)cc1 ; Cc1ccc(N2c3ccc(C)cc3B3c4cc(-c5ccccc5)ccc4N(c4ccc(-c5ccccc5)cc4)c4cccc2c43)cc1]

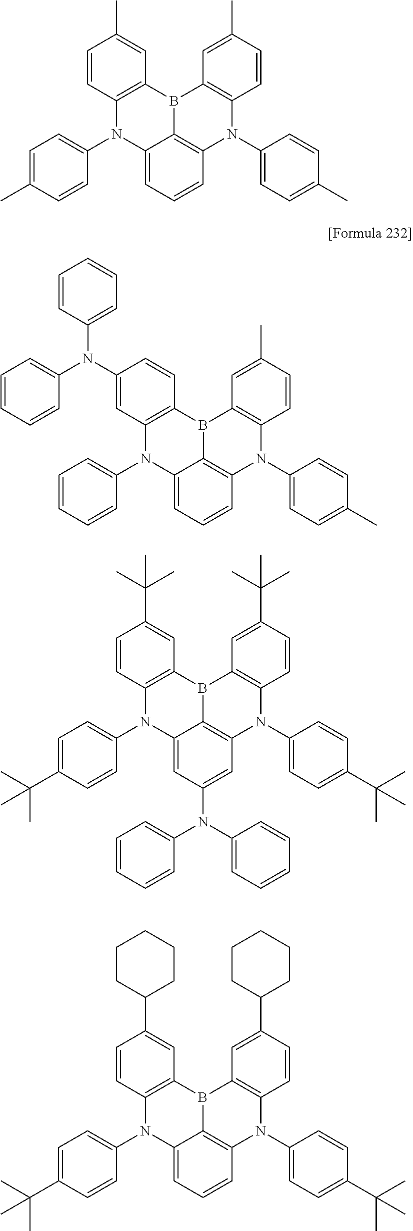

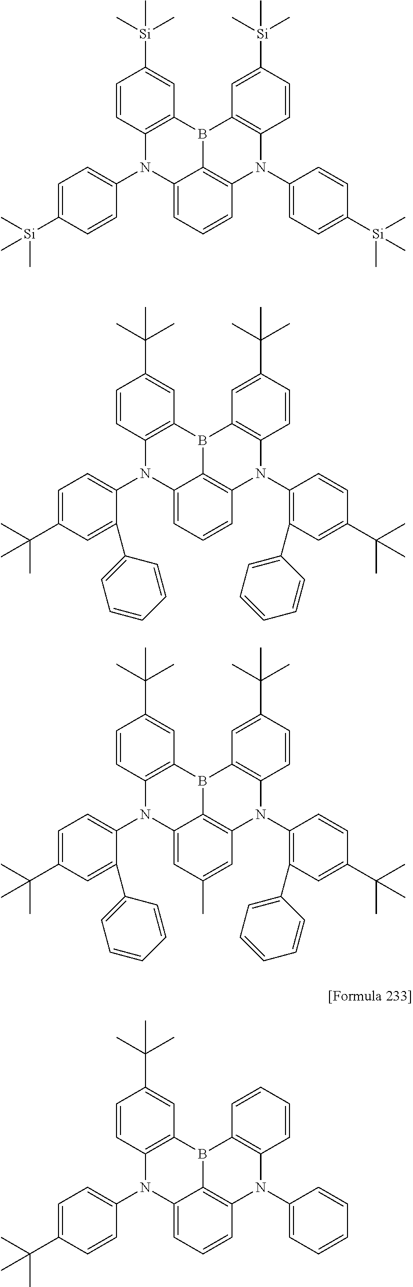

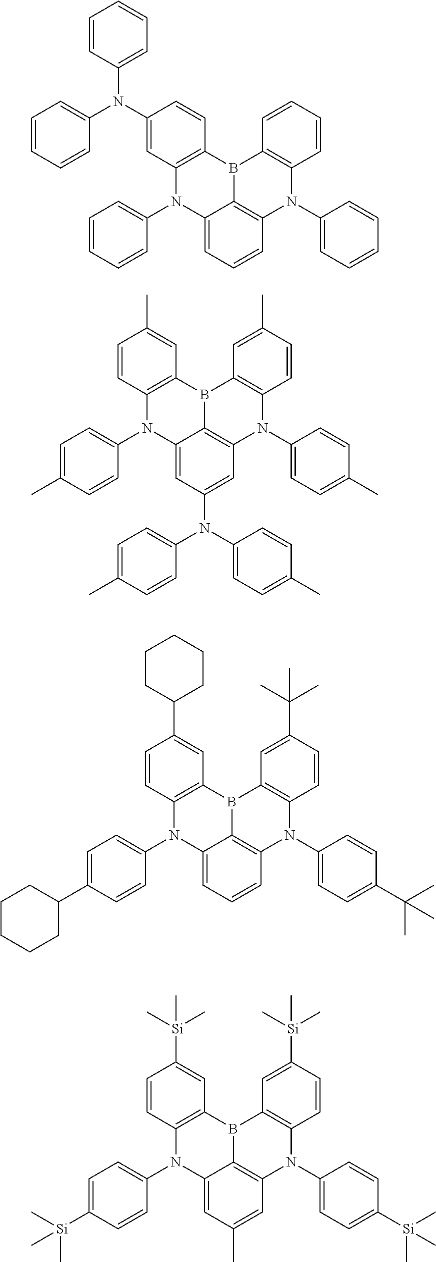

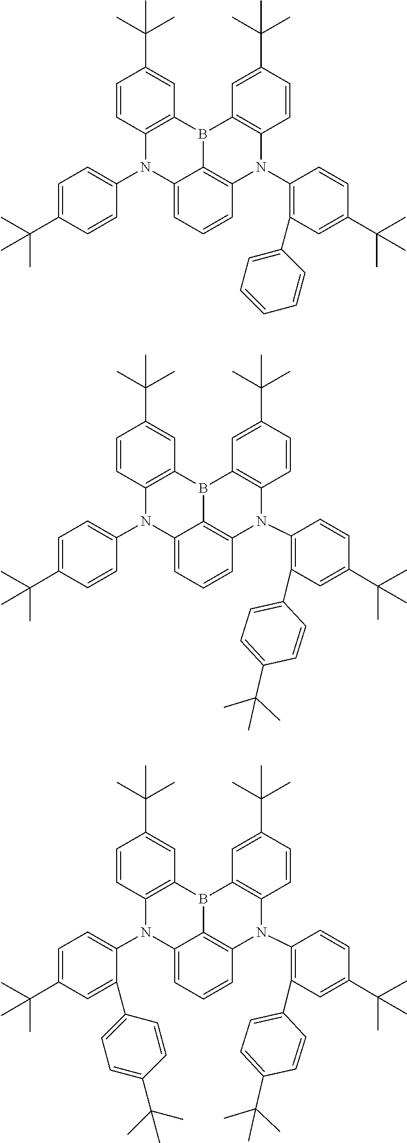

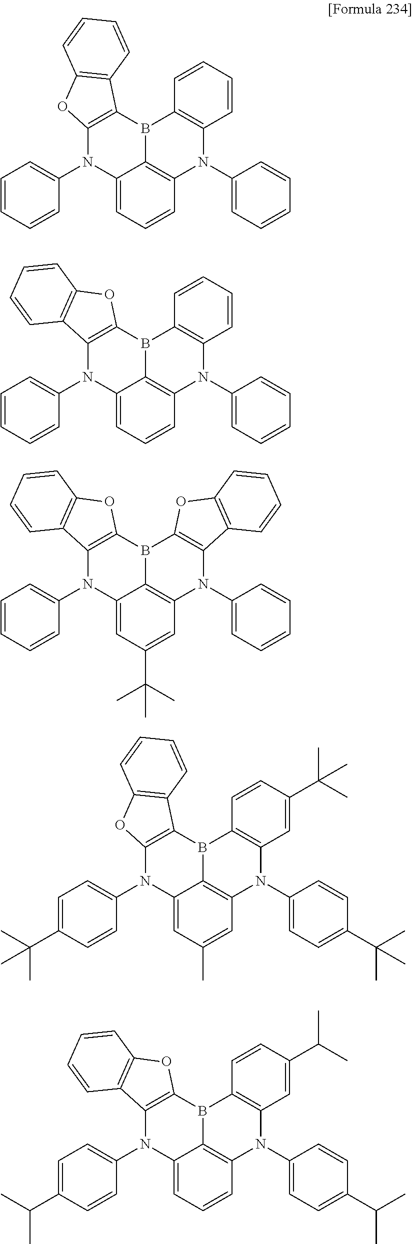

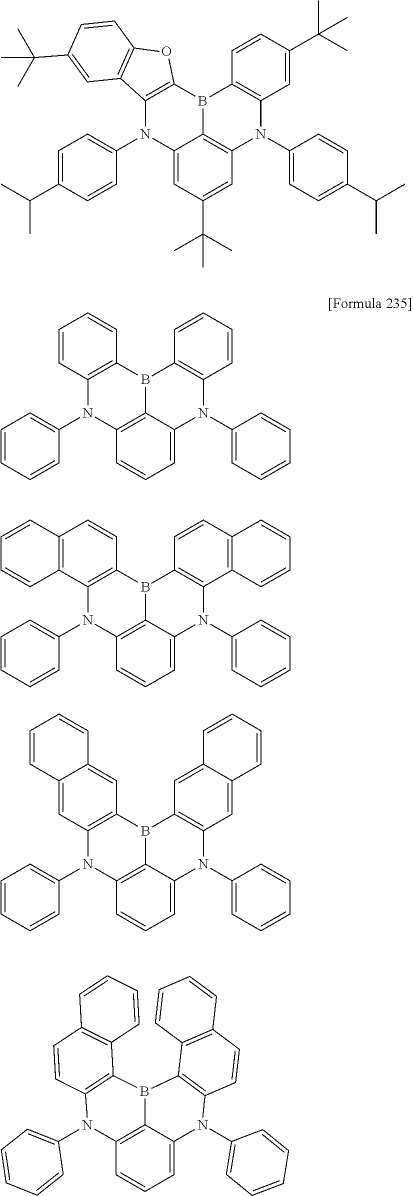

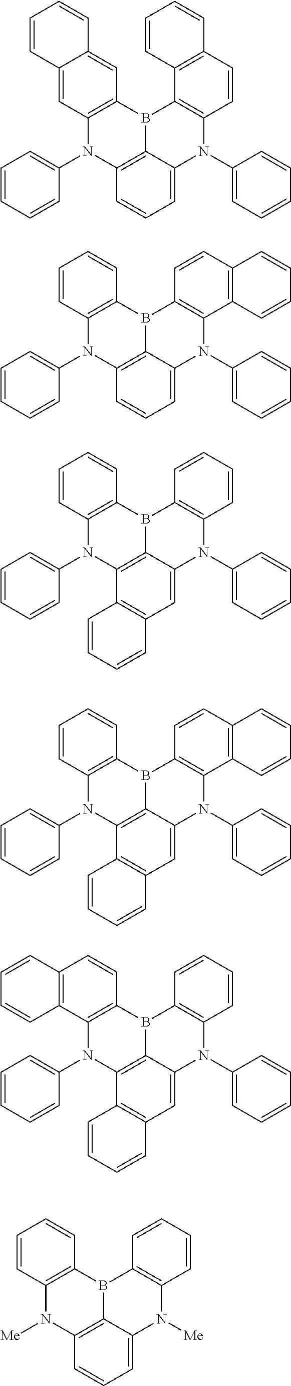

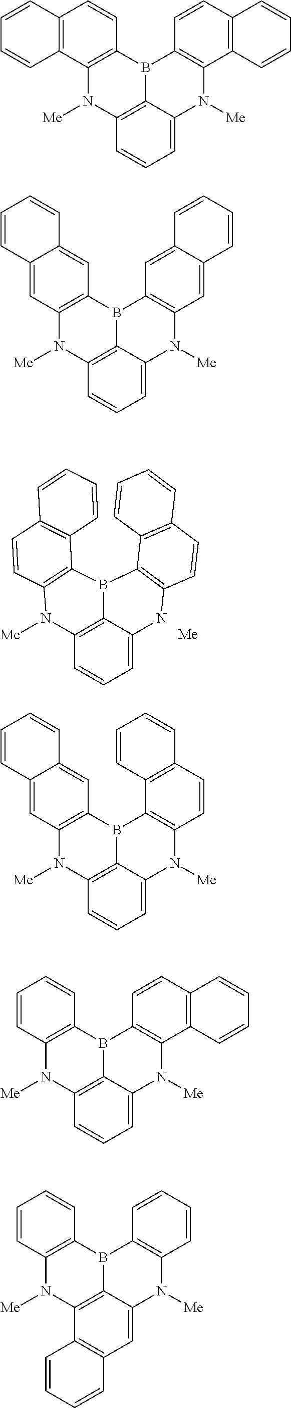

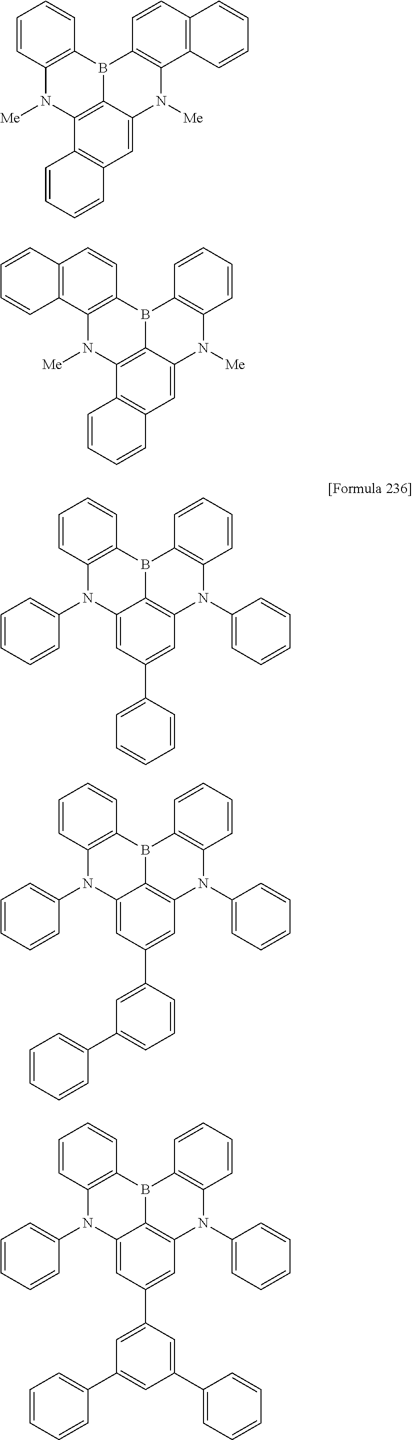

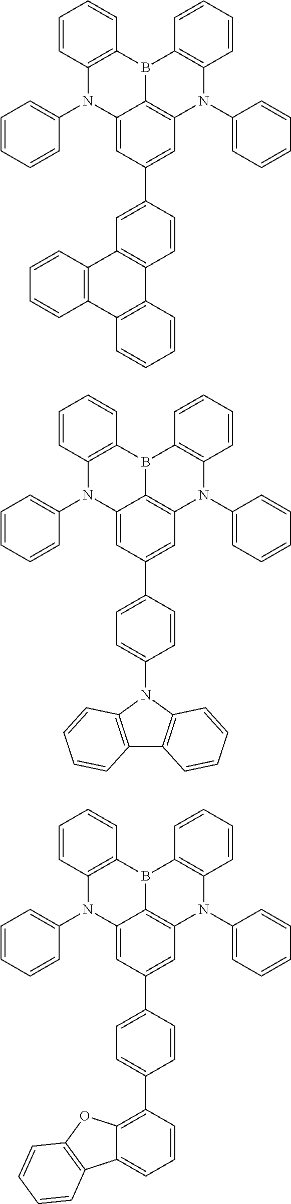

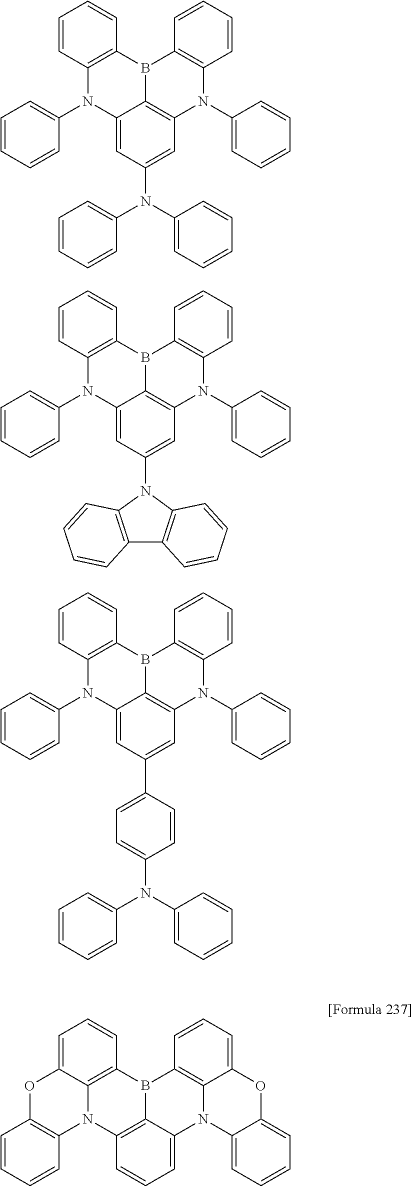

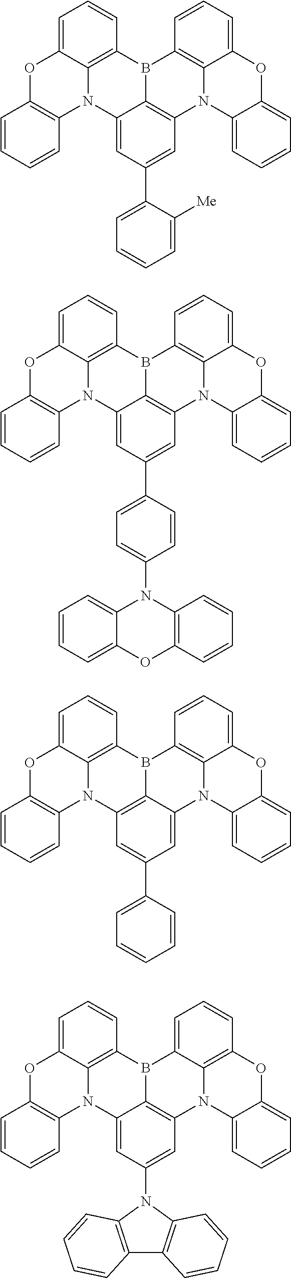

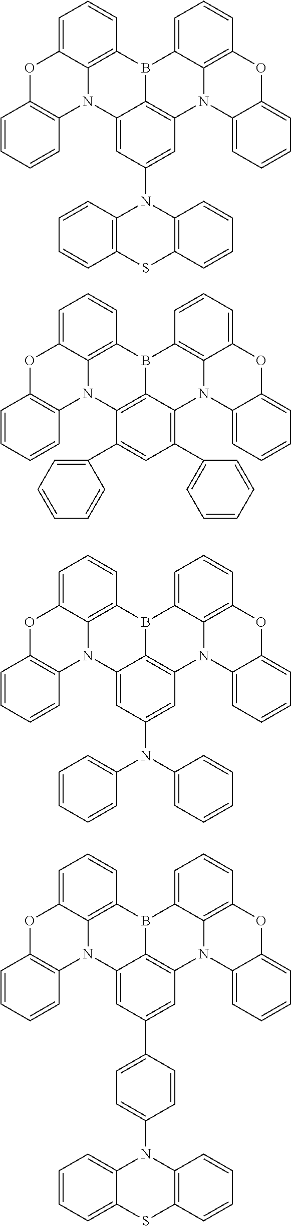

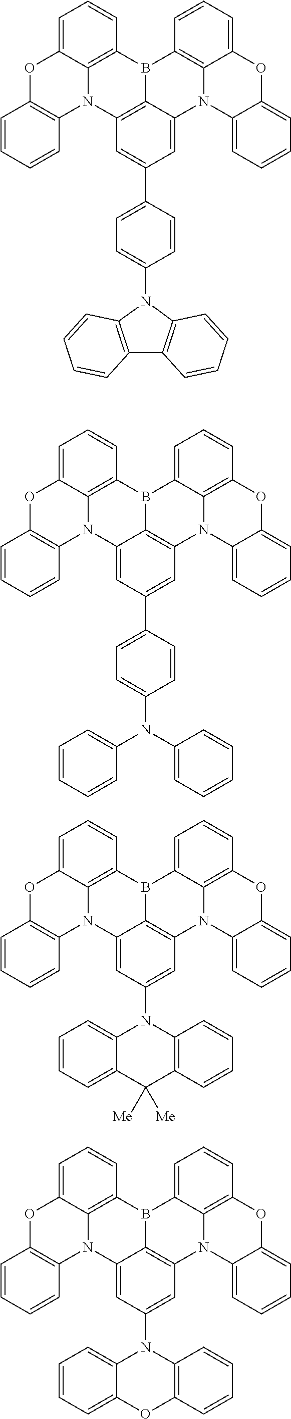

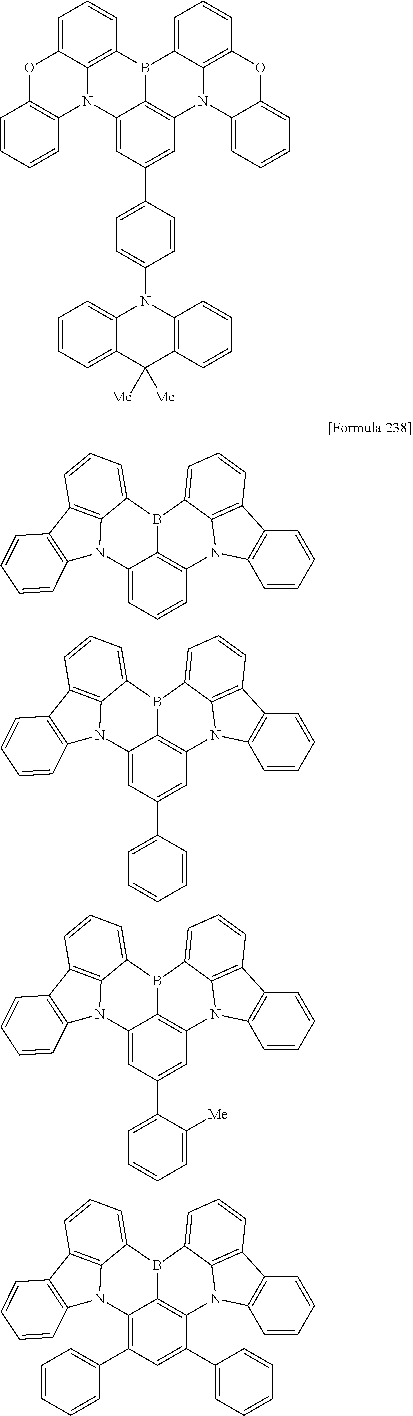

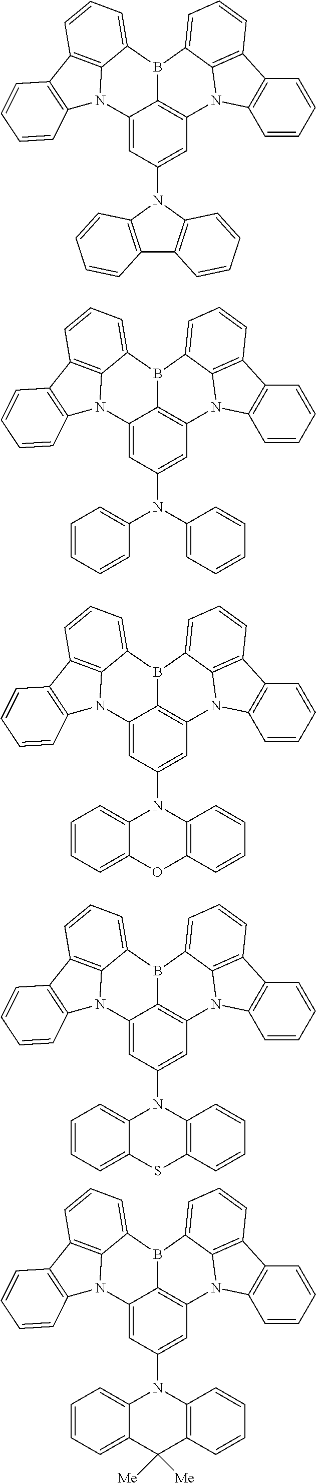

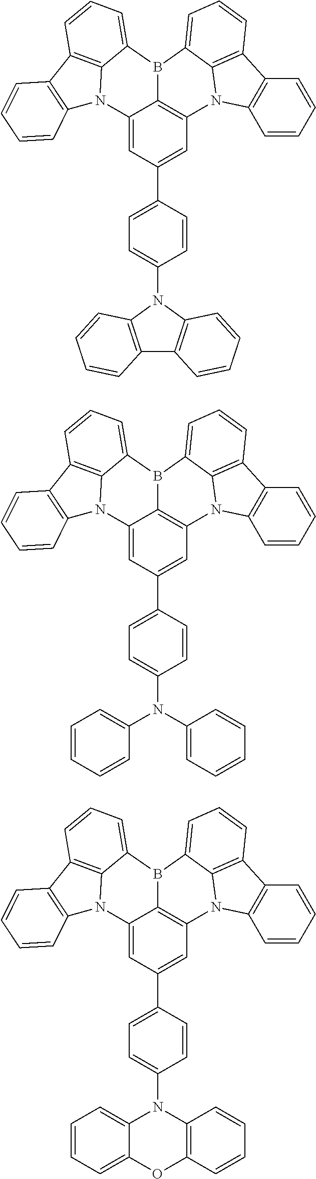

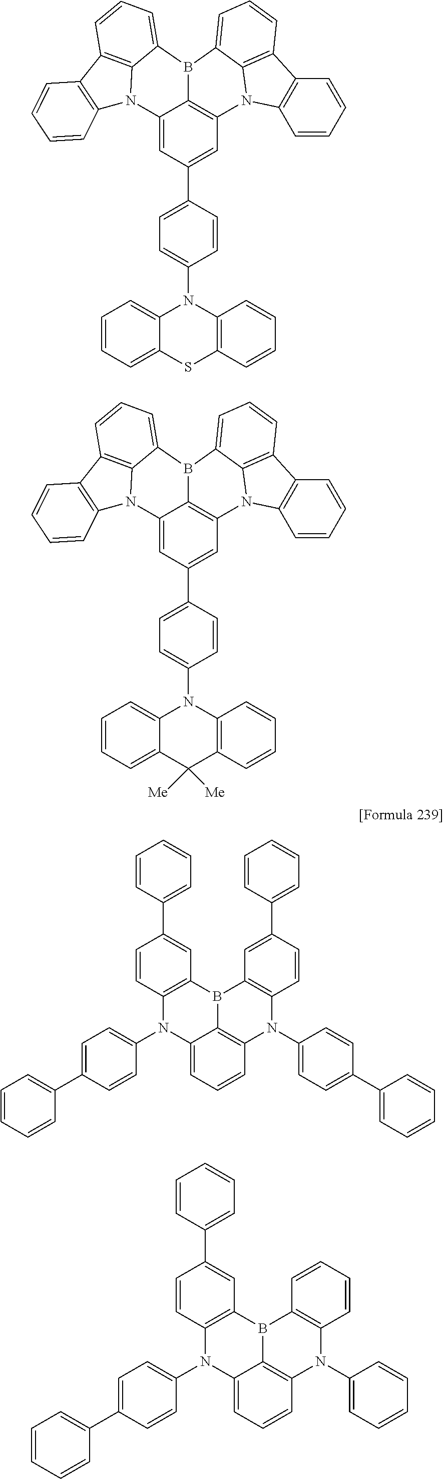

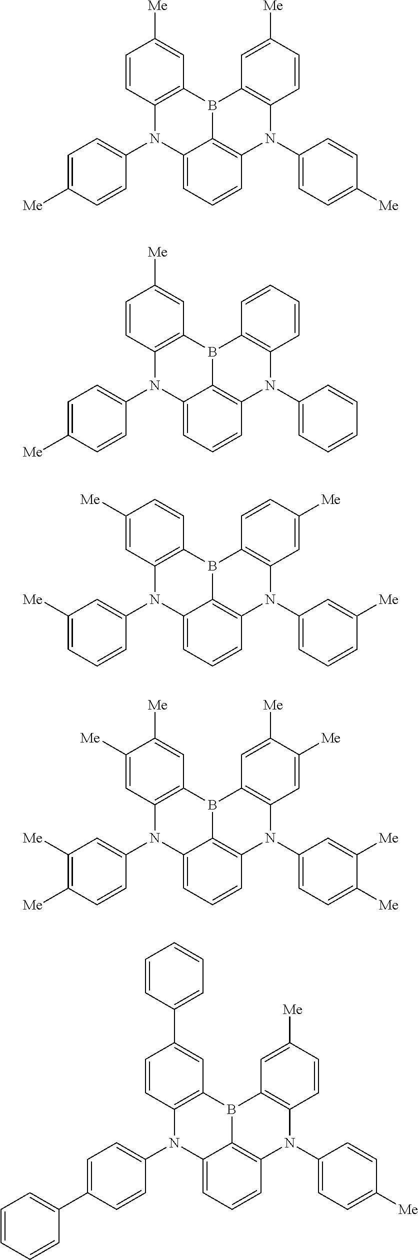 [SMILES: Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1]

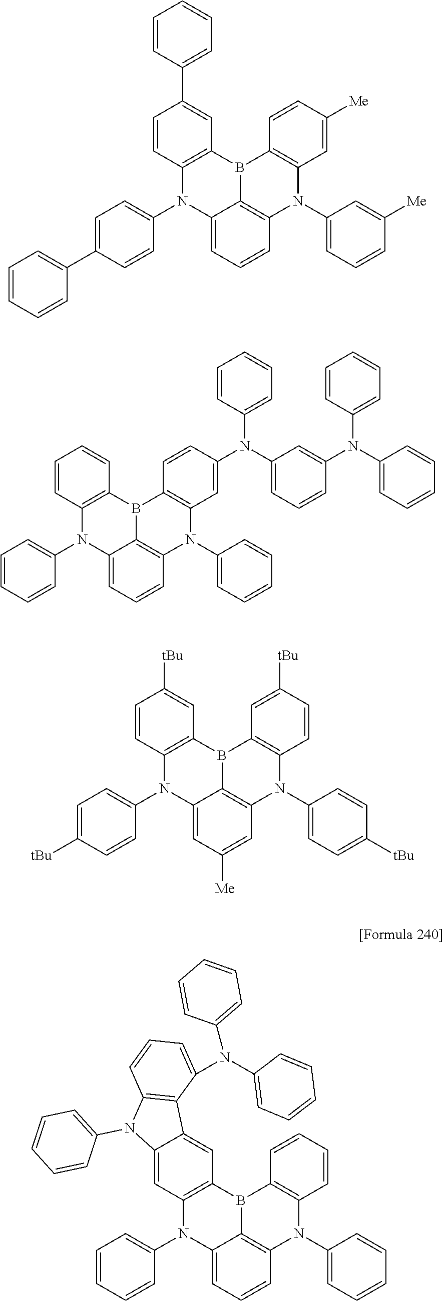 [SMILES: Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1]

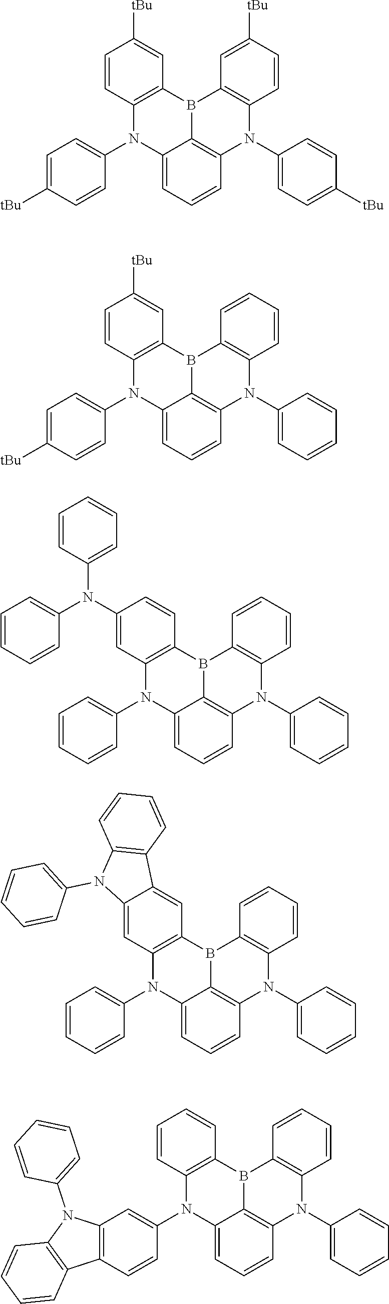

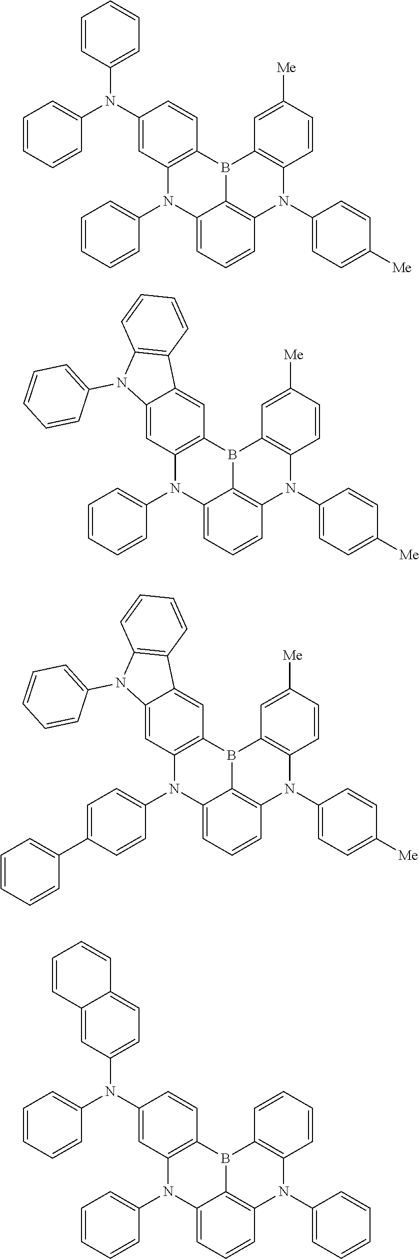

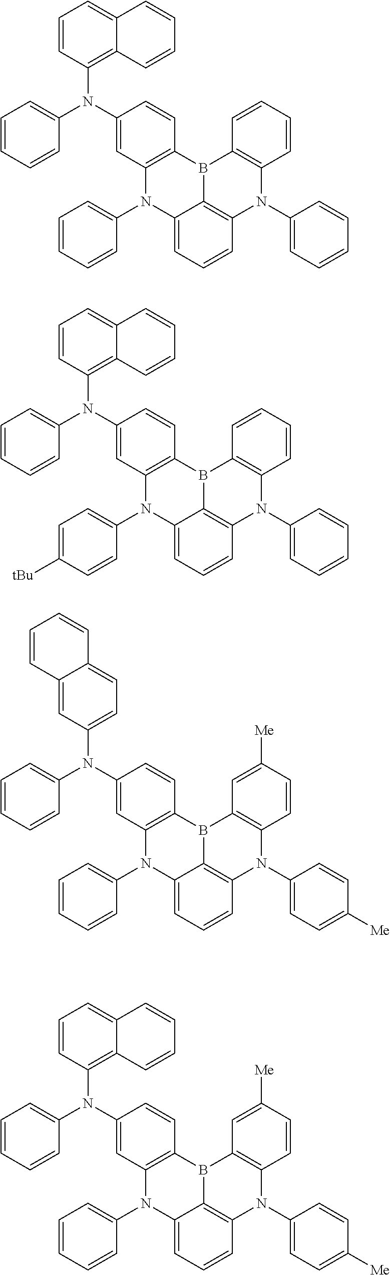

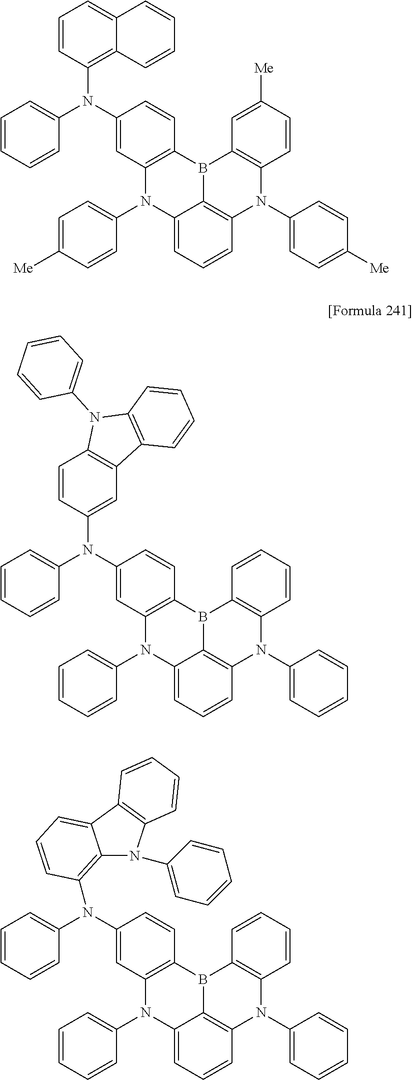 [SMILES: Cc1ccc(N2c3ccc(C)cc3B3c4ccc(N(c5ccccc5)c5cccc6ccccc56)cc4N(c4ccc(C)cc4)c4cccc2c43)cc1 ; c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1]

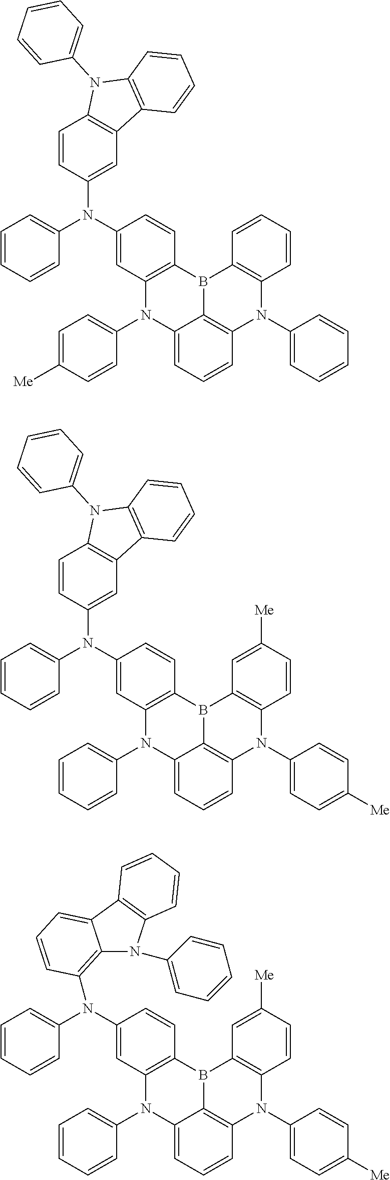 [SMILES: Cc1ccc(N2c3cc(N(c4ccccc4)c4ccc5c(c4)c4ccccc4n5-c4ccccc4)ccc3B3c4ccccc4N(c4ccccc4)c4cccc2c43)cc1 ; Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1]

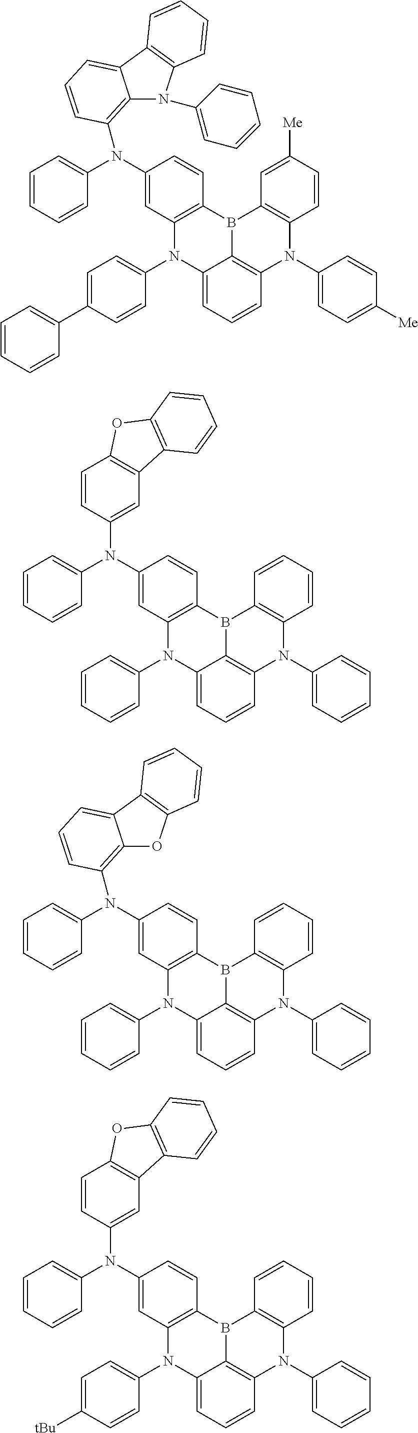

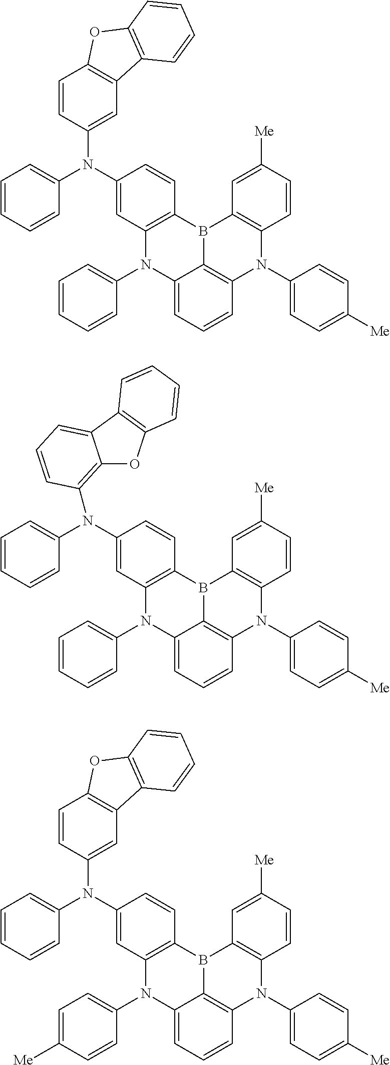 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

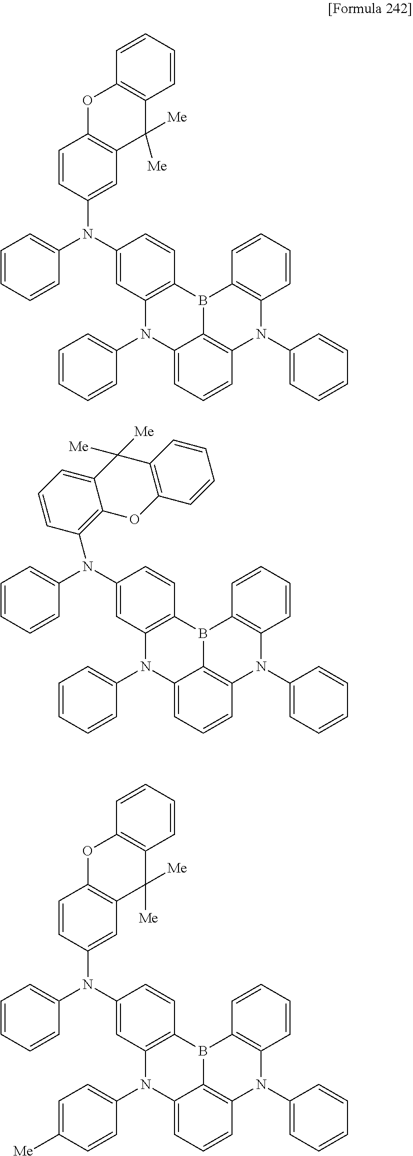

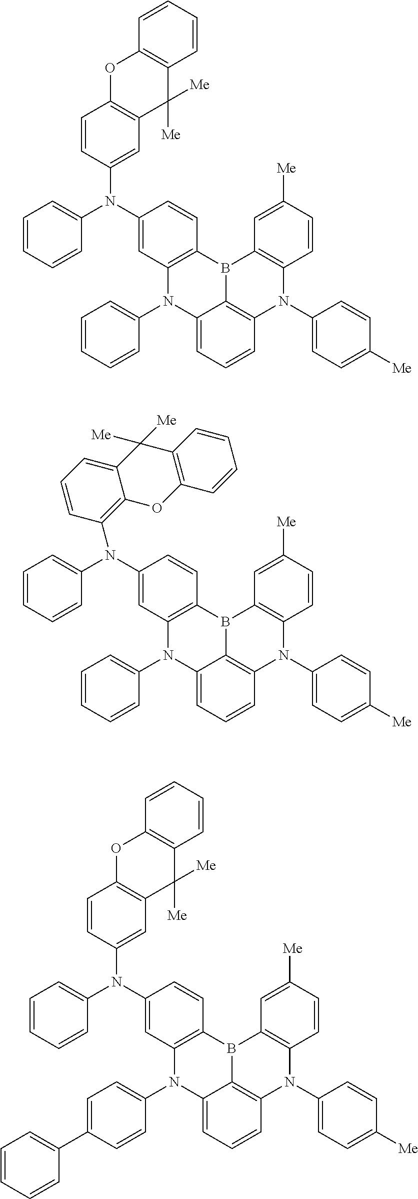 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

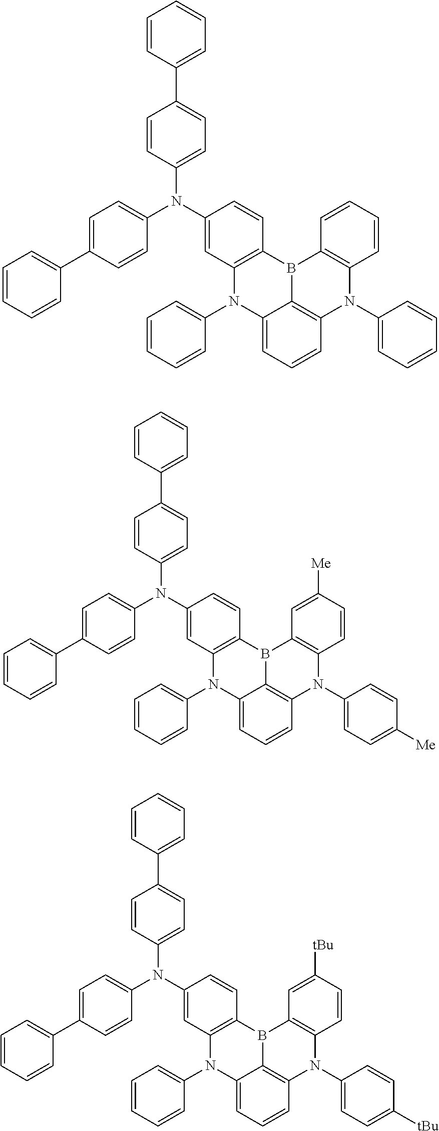 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1]

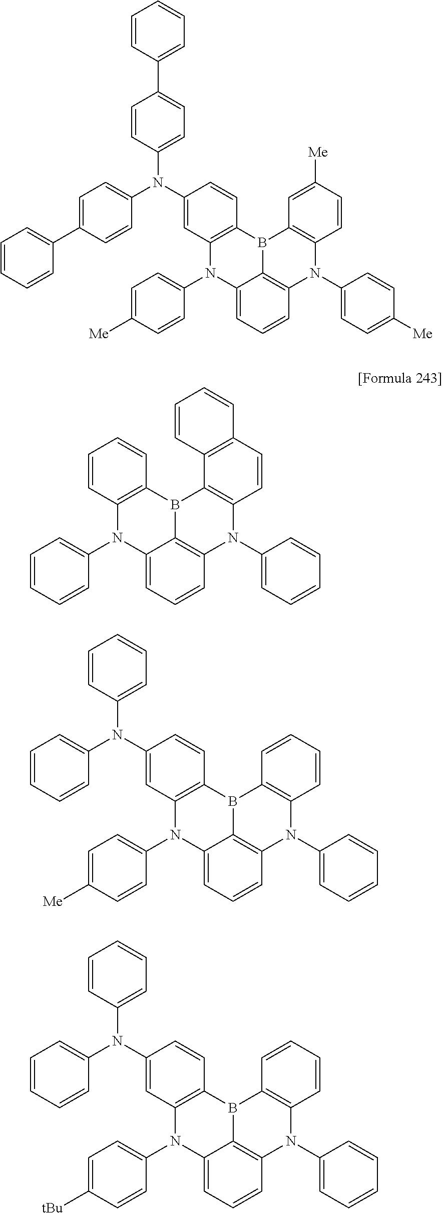

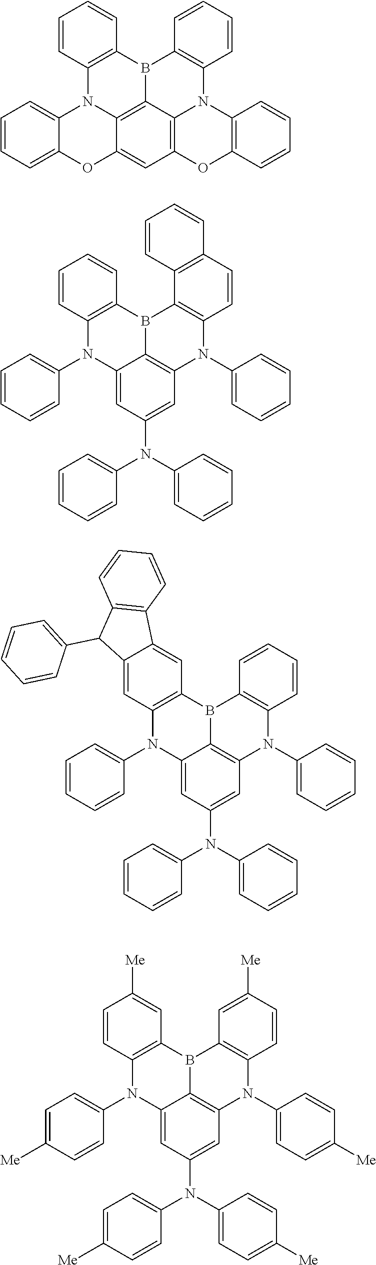

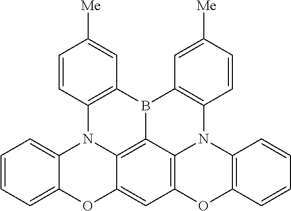 [SMILES: Cc1ccc2c(c1)B1c3cc(C)ccc3-n3c4ccccc4oc4cc5oc6ccccc6n-2c5c1c43]

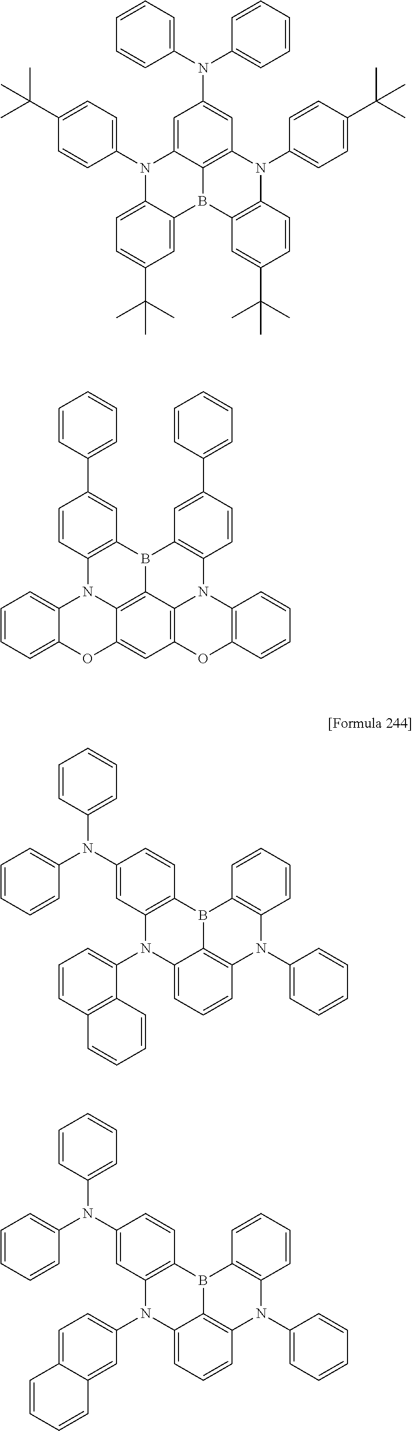 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; c1ccc(-c2ccc3c(c2)B2c4cc(-c5ccccc5)ccc4-n4c5ccccc5oc5cc6oc7ccccc7n-3c6c2c54)cc1 ; c1ccc(N(c2ccccc2)c2ccc3c(c2)N(c2cccc4ccccc24)c2cccc4c2B3c2ccccc2N4c2ccccc2)cc1 ; c1ccc(N(c2ccccc2)c2ccc3c(c2)N(c2ccc4ccccc4c2)c2cccc4c2B3c2ccccc2N4c2ccccc2)cc1]

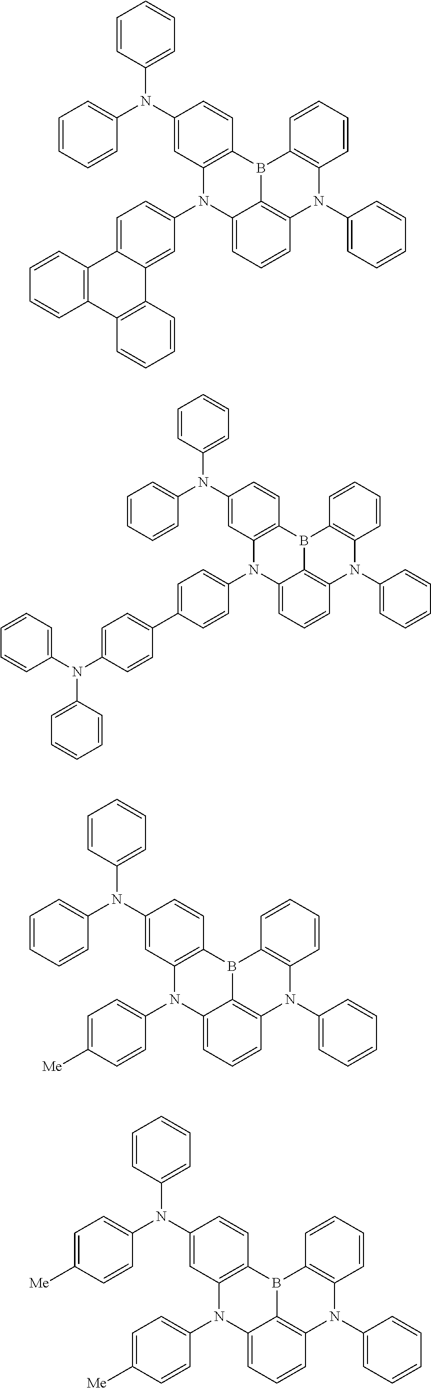 [SMILES: c1ccc(N(c2ccccc2)c2ccc3c(c2)N(c2ccc4c5ccccc5c5ccccc5c4c2)c2cccc4c2B3c2ccccc2N4c2ccccc2)cc1 ; c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1]

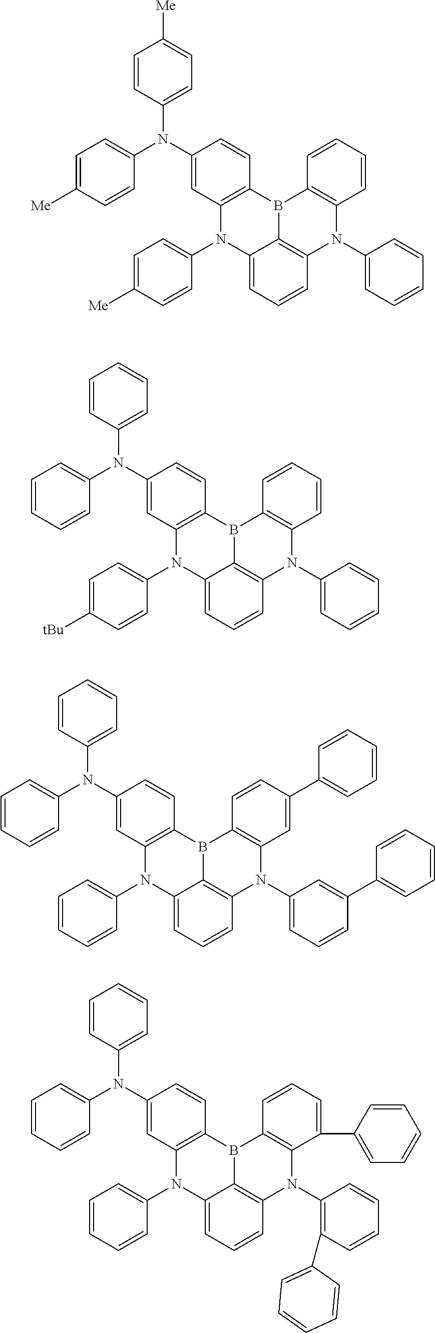 [SMILES: Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

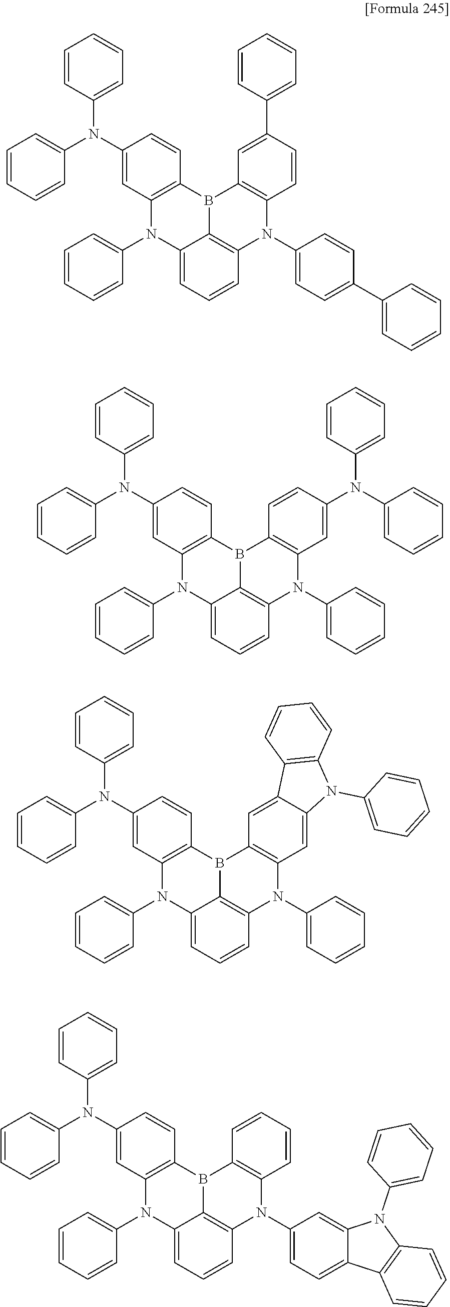 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1 ; c1ccc(N(c2ccccc2)c2ccc3c(c2)N(c2ccccc2)c2cccc4c2B3c2ccccc2N4c2ccc3c4ccccc4n(-c4ccccc4)c3c2)cc1]

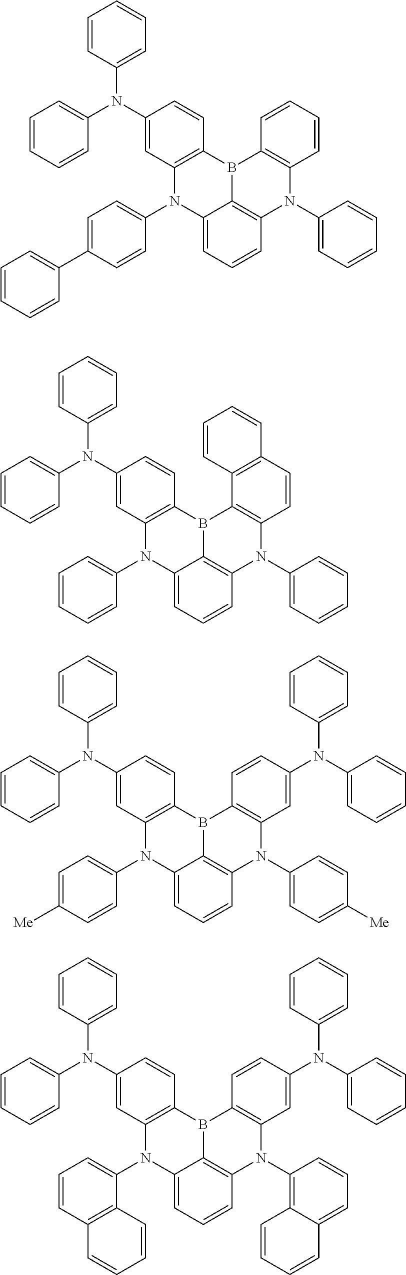

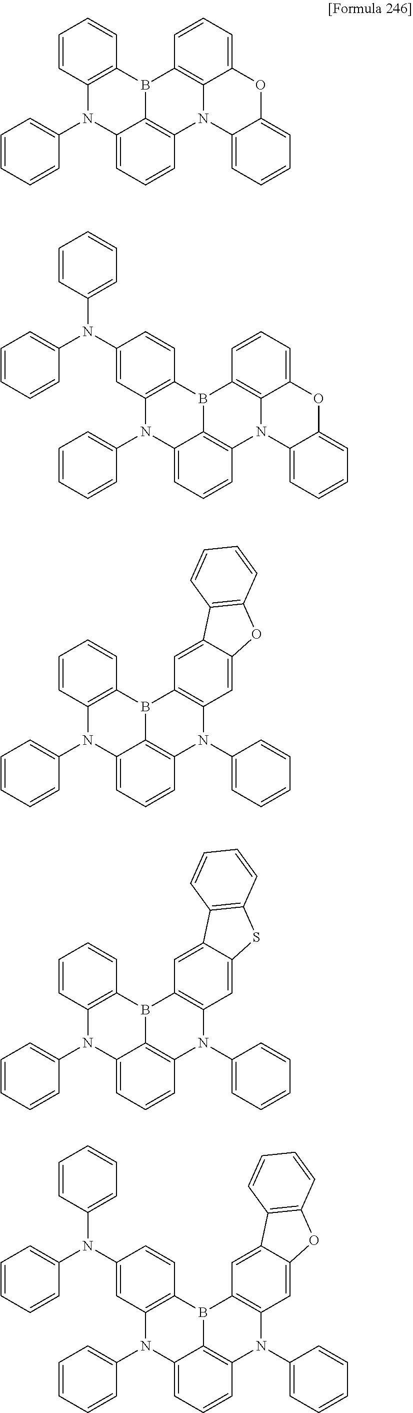

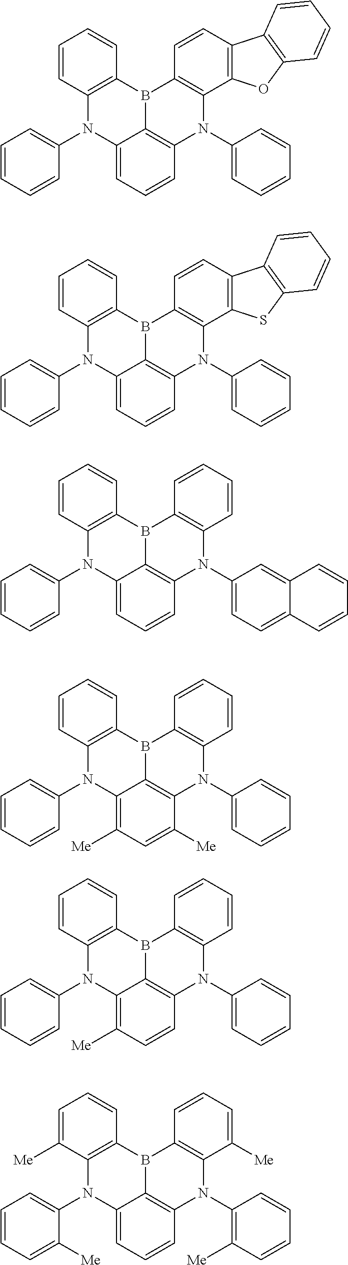

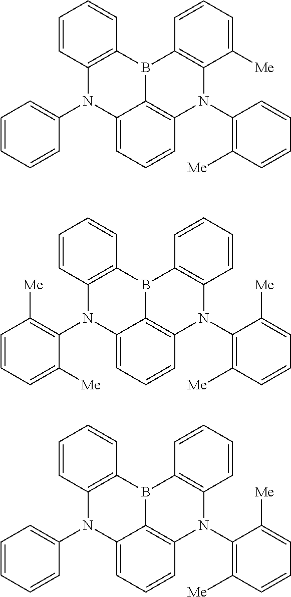

Compound Represented by Formula (7)

A compound represented by the formula (7) is described below.

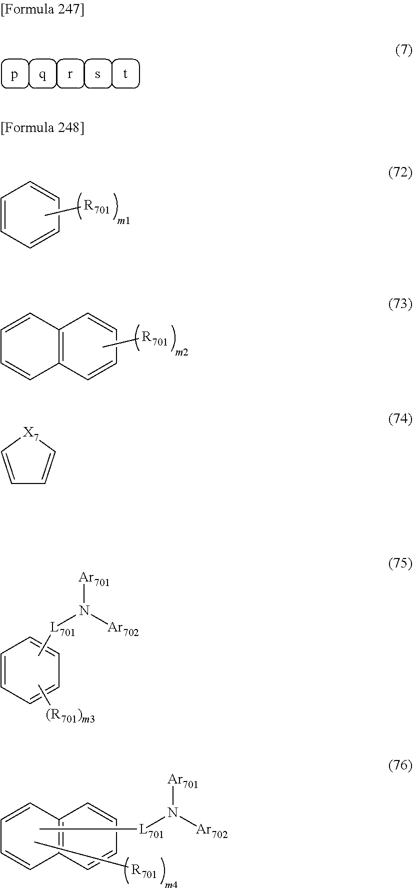

In the formula (7),
the r ring is a ring represented by the formula (72) or the formula (73) and fused at any position of an adjacent ring,
the q ring and the s ring are each independently a ring represented by the formula (74) and fused at any position of an adjacent ring,
the p ring and t ring are each independently a structure represented by the formula (75) or the formula (76) and fused at any position of an adjacent ring, and
X₇ represents an oxygen atom, a sulfur atom, or NR₇₀₂.

When a plurality of R₇₀₁ are present, a plurality of adjacent R₇₀₁ are mutually bonded to form a substituted or unsubstituted monocyclic ring, are mutually bonded to form a substituted or unsubstituted fused ring, or are not mutually bonded,
R₇₀₁ and R₇₀₂ that do not form the monocyclic ring and do not form the fused ring each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms,
Ar₇₀₁ and Ar₇₀₂ each independently represent a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,
L₇₀₁ represents a substituted or unsubstituted alkylene group having 1 to 50 carbon atoms, a substituted or unsubstituted alkenylene group having 2 to 50 carbon atoms, a substituted or unsubstituted alkynylene group having 2 to 50 carbon atoms, a substituted or unsubstituted cycloalkylene group having 3 to 50 ring carbon atoms, a substituted or unsubstituted arylene group having 6 to 50 ring carbon atoms, or a substituted or unsubstituted divalent heterocyclic group having 5 to 50 ring atoms,
m1 is 0, 1, or 2,
m2 is 0, 1, 2, 3, or 4,
m3 is each independently 0, 1, 2, or 3,
m4 is each independently 0, 1, 2, 3, 4, or 5,
when a plurality of R₇₀₁ are present, the plurality of R₇₀₁ are mutually the same or different,
when a plurality of X₇ are present, the plurality of X₇ are mutually the same or different,
when a plurality of R₇₀₂ are present, the plurality of R₇₀₂ are mutually the same or different,
when a plurality of Ar₇₀₁ are present, the plurality of Ar₇₀₁ are mutually the same or different,
when a plurality of Ar₇₀₂ are present, the plurality of Ar₇₀₂ are mutually the same or different, and
when a plurality of L₇₀₁ are present, the plurality of L₇₀₁ are mutually the same or different.

In the formula (7), each of the p ring, the q ring, the r ring, the s ring, and the t ring is fused to an adjacent ring by sharing two carbon atoms. The position and direction where the rings are fused are not particularly limited. The rings may be fused at any position and in any direction.

In an exemplary embodiment, in the r ring represented by the formula (72) or the formula (73), m1 is 0, or m2 is 0.

In an exemplary embodiment, a compound represented by the formula (7) is represented by one of the following formulae (71-1) to (71-6).

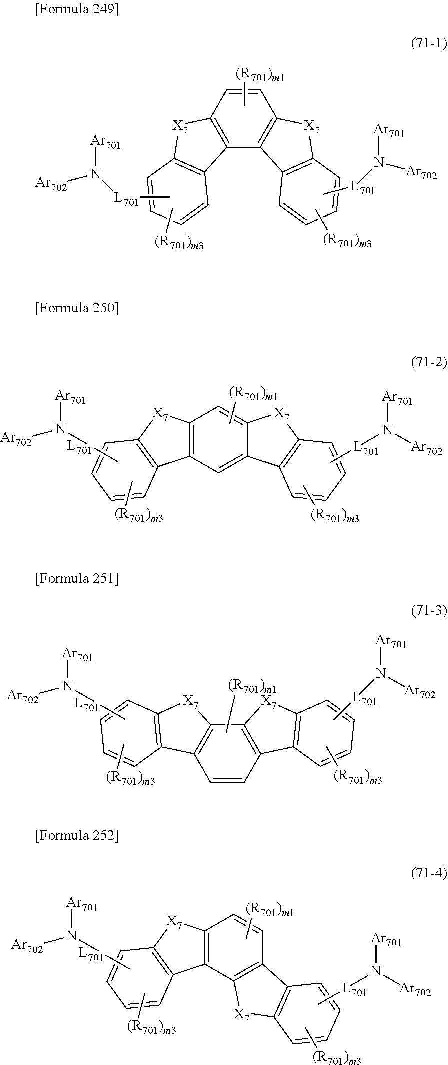

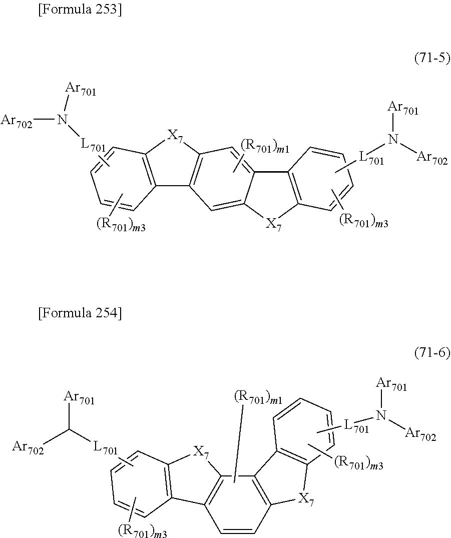

In the formulae (71-1) to (71-6), R₇₀₁, X₇, Ar₇₀₁, Ar₇₀₂, L₇₀₁, m1, and m3 represent the same as R₇₀₁, X₇, Ar₇₀₁, Ar₇₀₂, L₇₀₁, m1, and m3, respectively, in the formula (7).

In an exemplary embodiment, a compound represented by the formula (7) is represented by one of the following formulae (71-11) to (71-13).

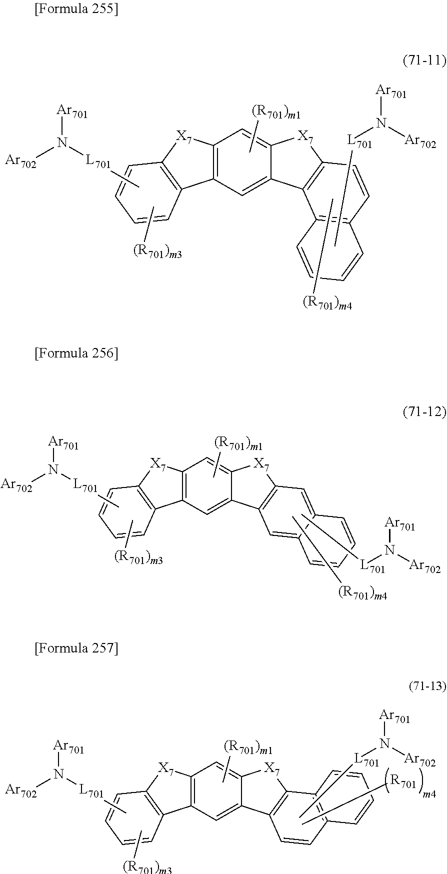

In the formula (71-11) to the formula (71-13), R₇₀₁, X₇, Ar₇₀₁, Ar₇₀₂, L₇₀₁, m1, m3, and m4 represent the same as R₇₀₁, X₇, Ar₇₀₁, Ar₇₀₂, L₇₀₁, m1, m3, and m4, respectively, in the formula (7).

In an exemplary embodiment, a compound represented by the formula (7) is represented by one of the following formulae (71-21) to (71-25).

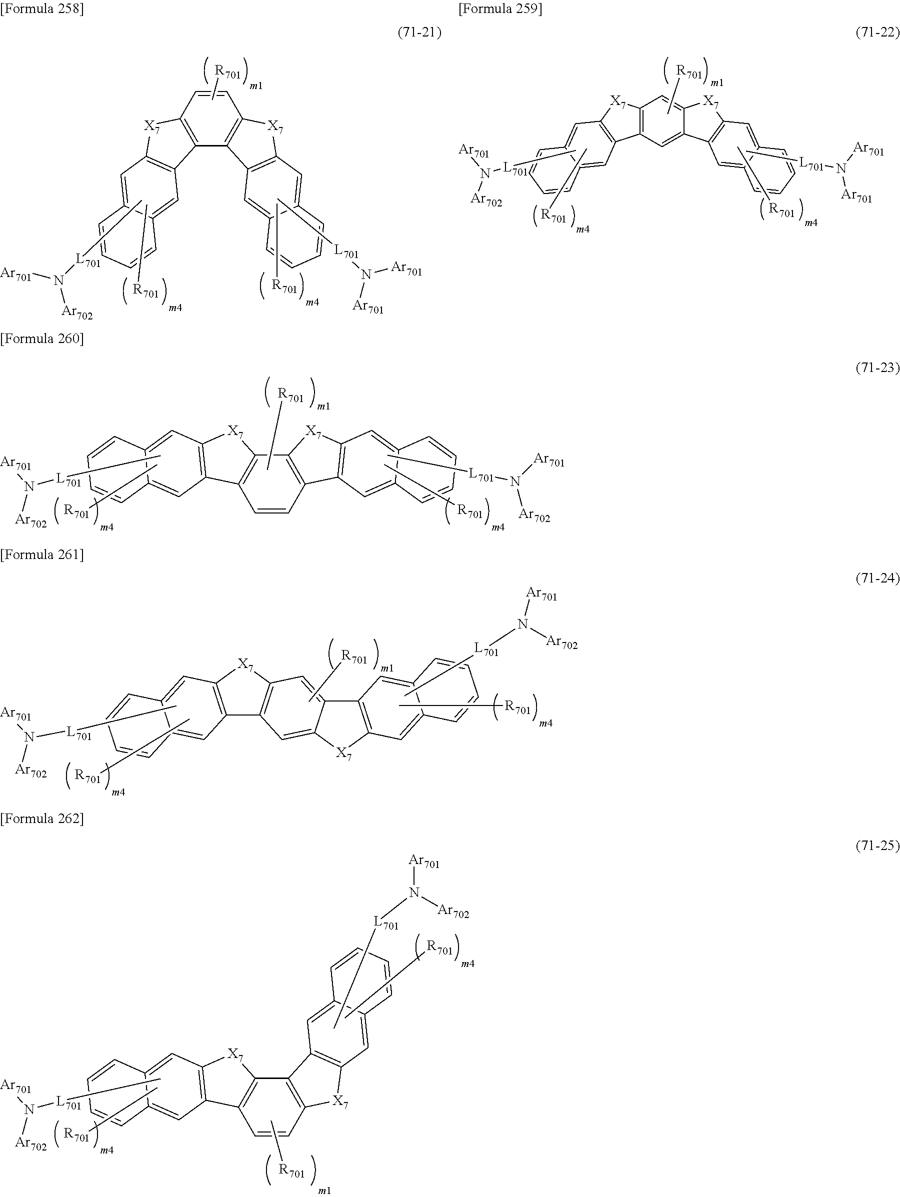

In the formula (71-21) to the formula (71-25), R₇₀₁, X₇, Ar₇₀₁, Ar₇₀₂, L₇₀₁, m1, and m4 represent the same as R₇₀₁, X₇, Ar₇₀₁, Ar₇₀₂, L₇₀₁, m1, and m4, respectively, in the formula (7).

In an exemplary embodiment, a compound represented by the formula (7) is represented by one of the following formulae (71-31) to (71-33).

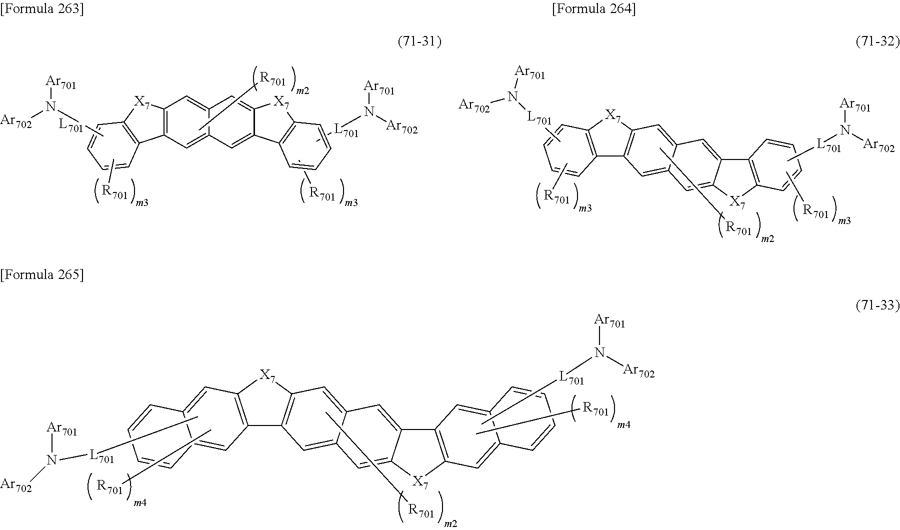

In the formula (71-31) to the formula (71-33), R₇₀₁, X₇, Ar₇₀₁, Ar₇₀₂, L₇₀₁, m2, to m4 represent the same as R₇₀₁, X₇, Ar₇₀₁, Ar₇₀₂, L₇₀₁, m2, to m4, respectively, in the formula (7).

In an exemplary embodiment, Ar₇₀₁ and Ar₇₀₂ each independently represent a substituted or unsubstituted aryl group having 6 to 50 ring carbon atoms.

In an exemplary embodiment, one of Ar₇₀₁ and Ar₇₀₂ represents a substituted or unsubstituted aryl group having 6 to 50 ring carbon atoms, and the other of Ar₇₀₁ and Ar₇₀₂ represents a substituted or unsubstituted heterocyclic group having 5 to 50 ring atoms.

Specific examples of a compound represented by the formula (7) include the following compounds.

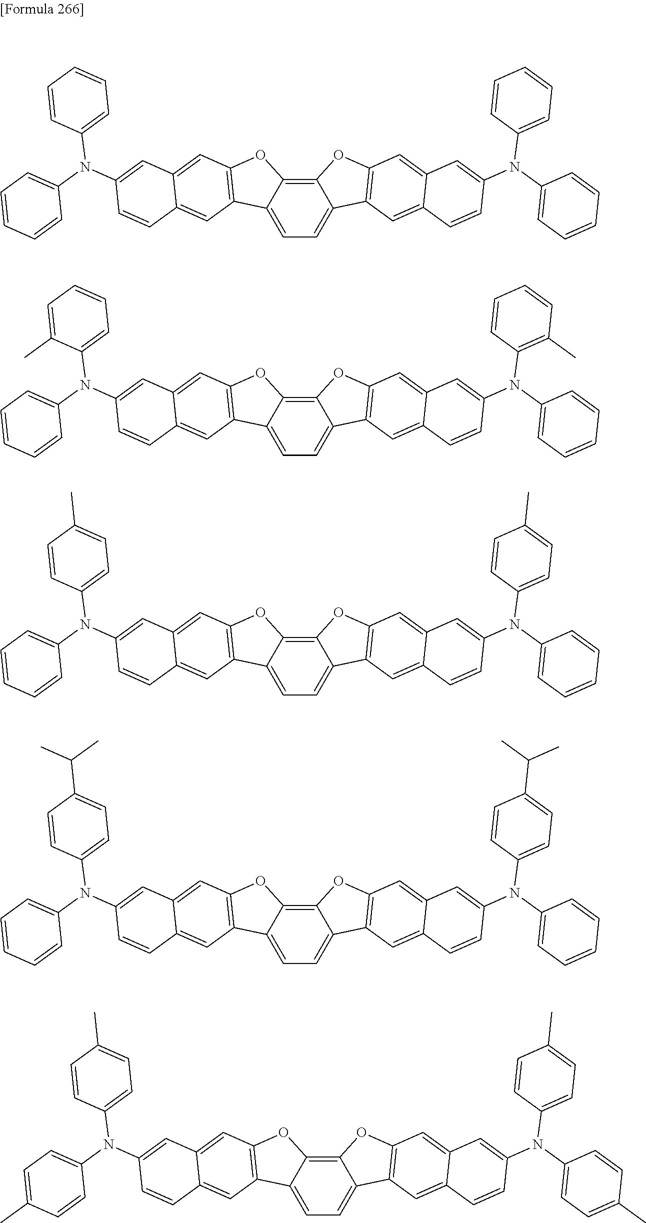

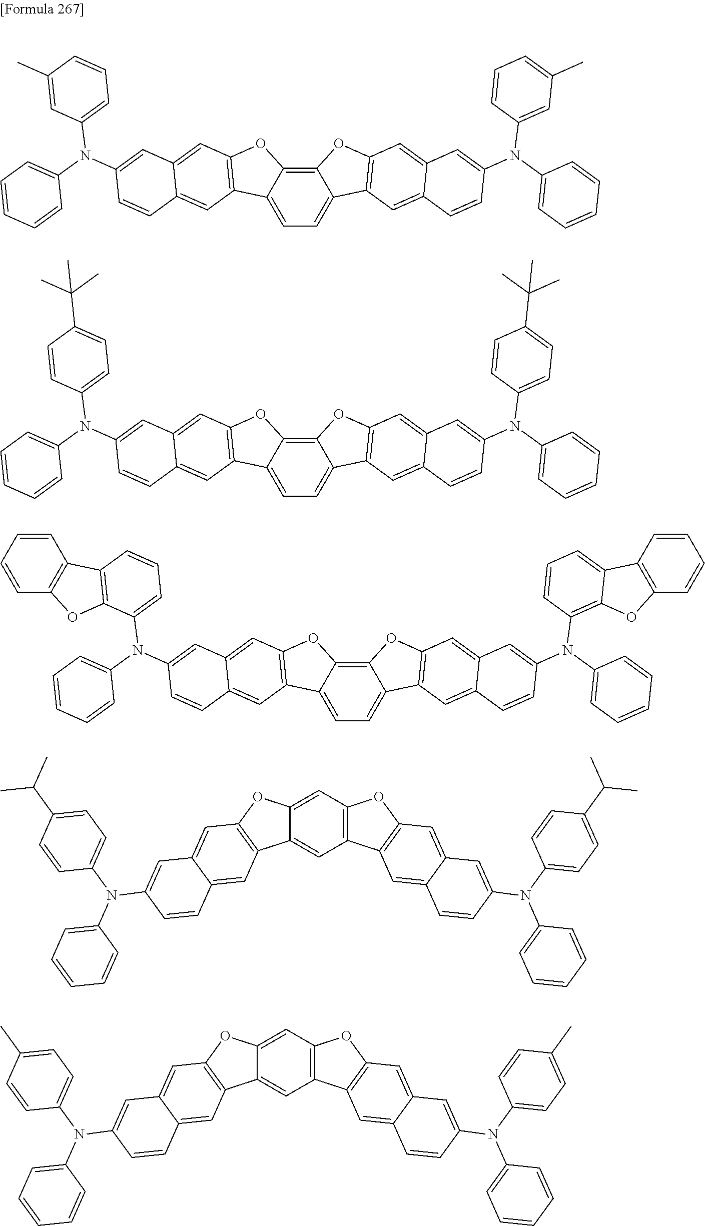

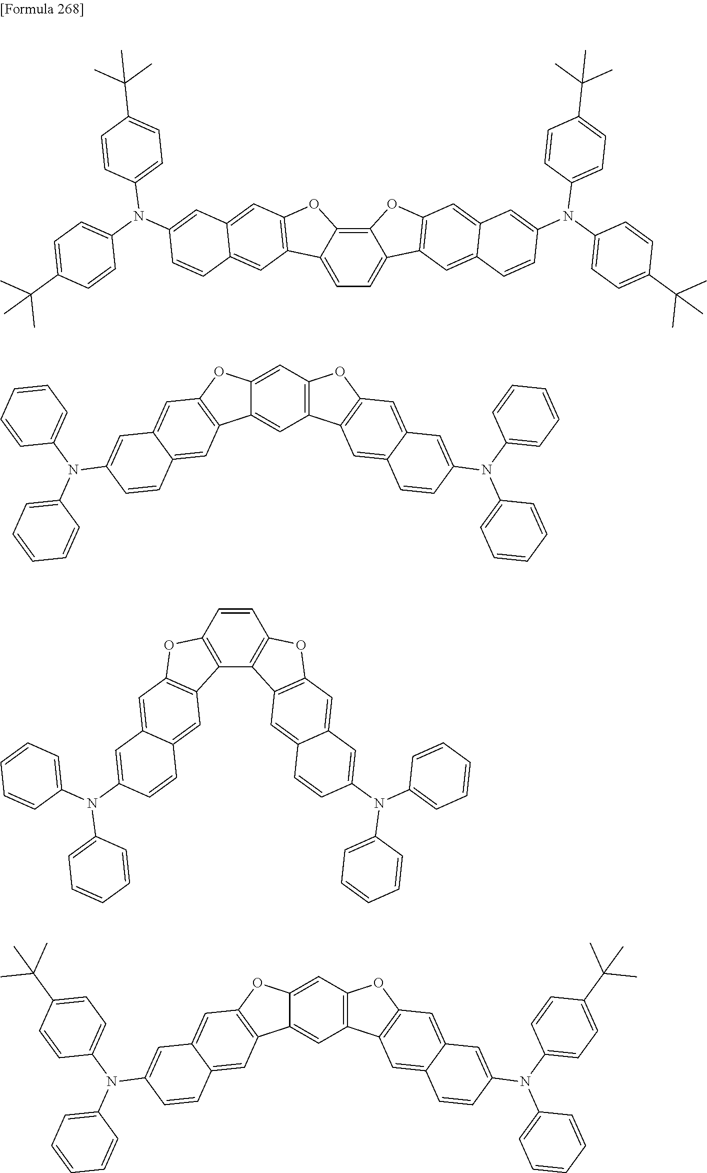

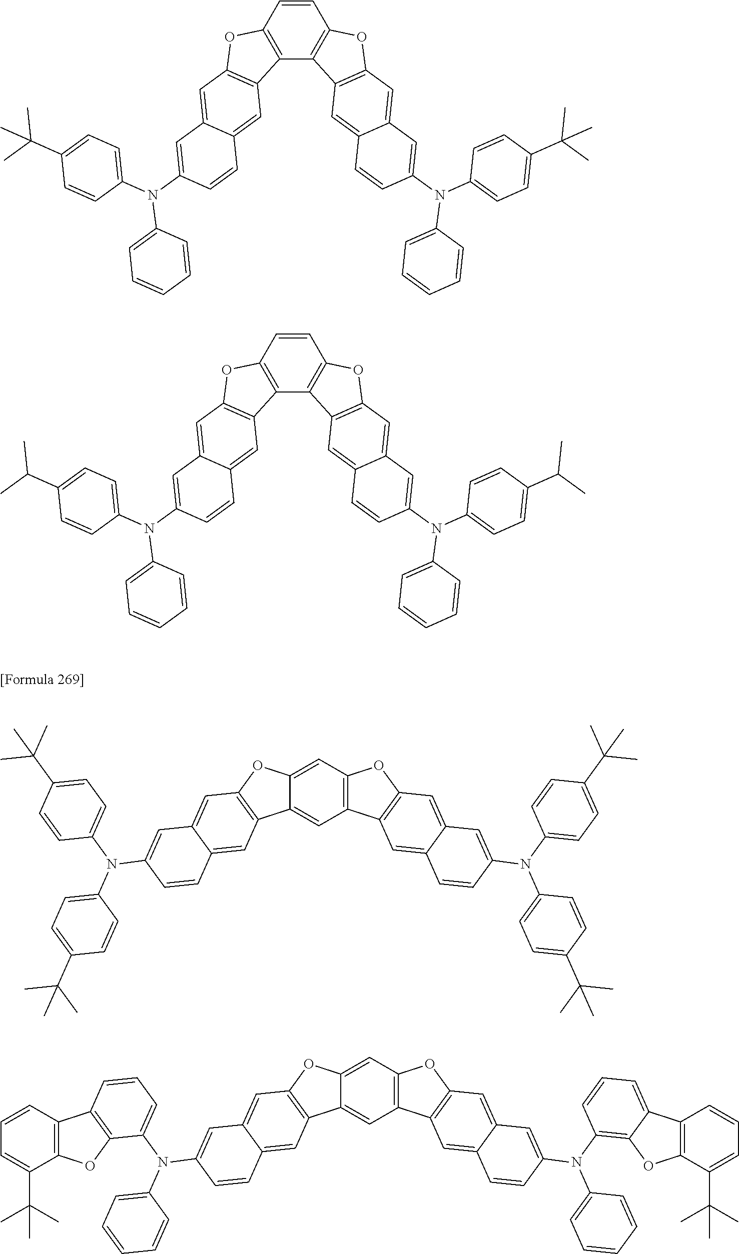

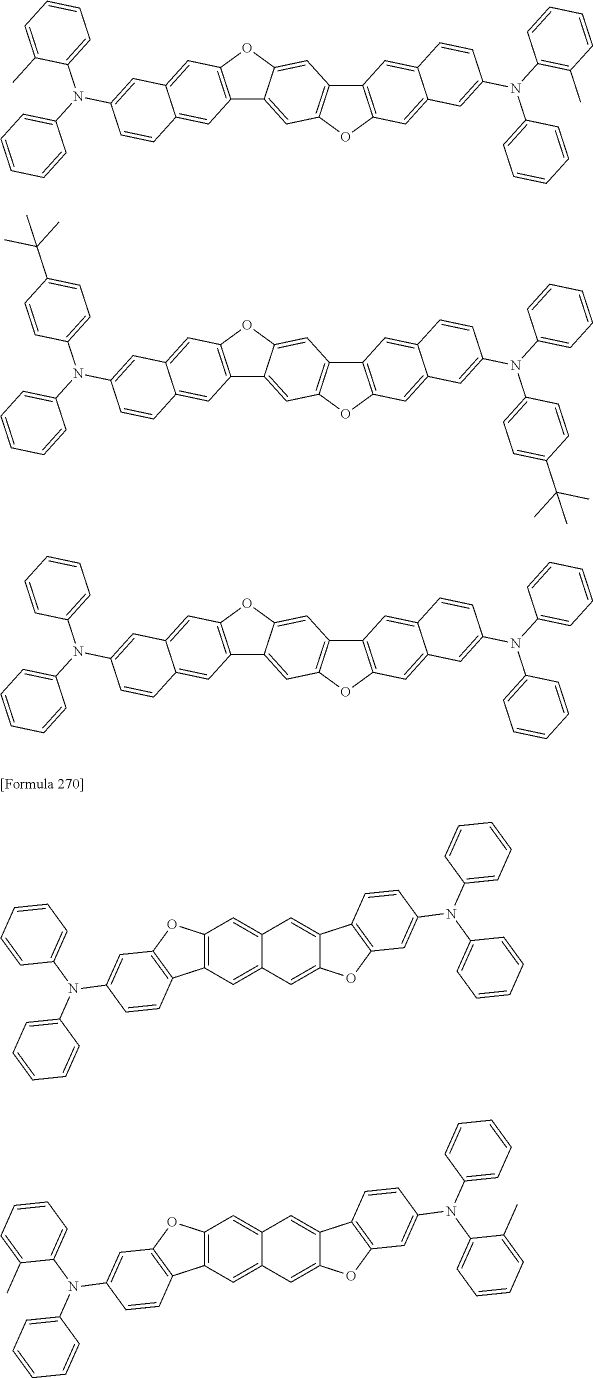

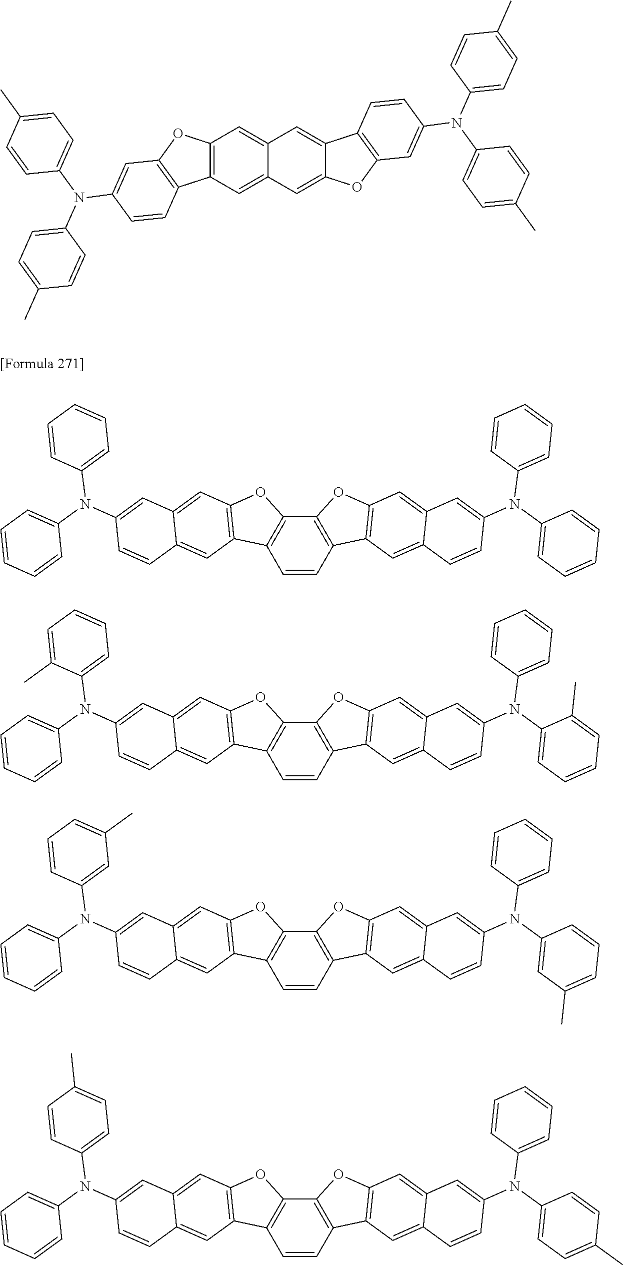

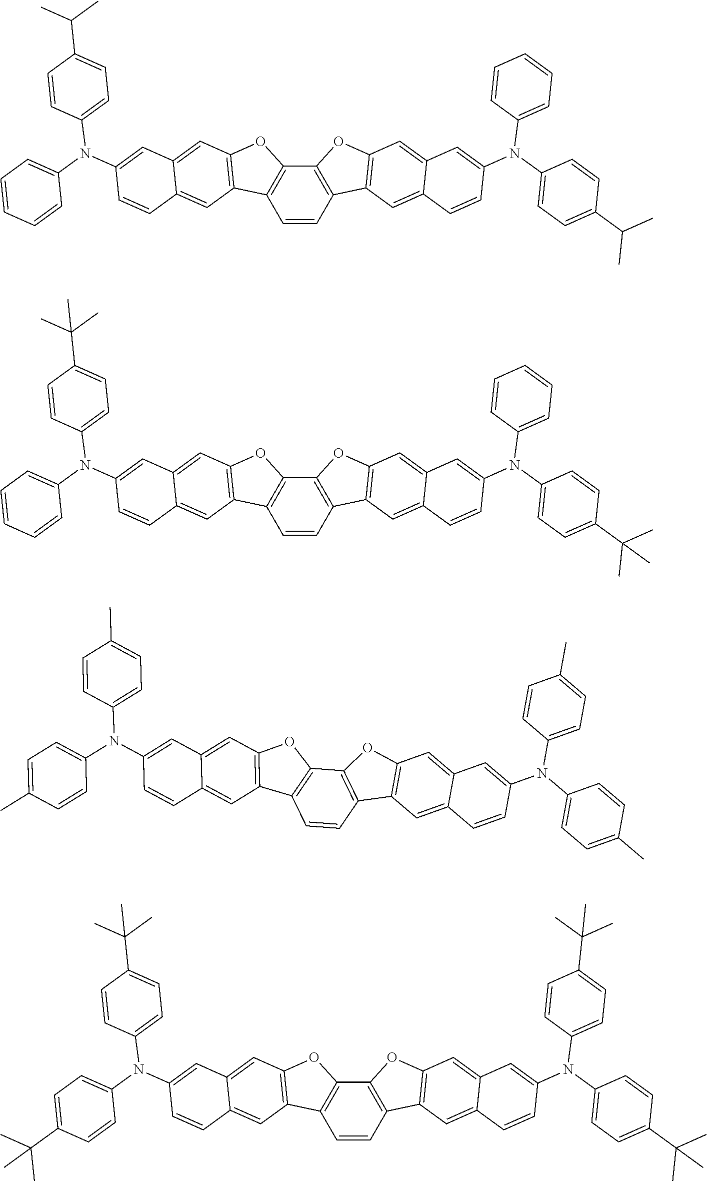

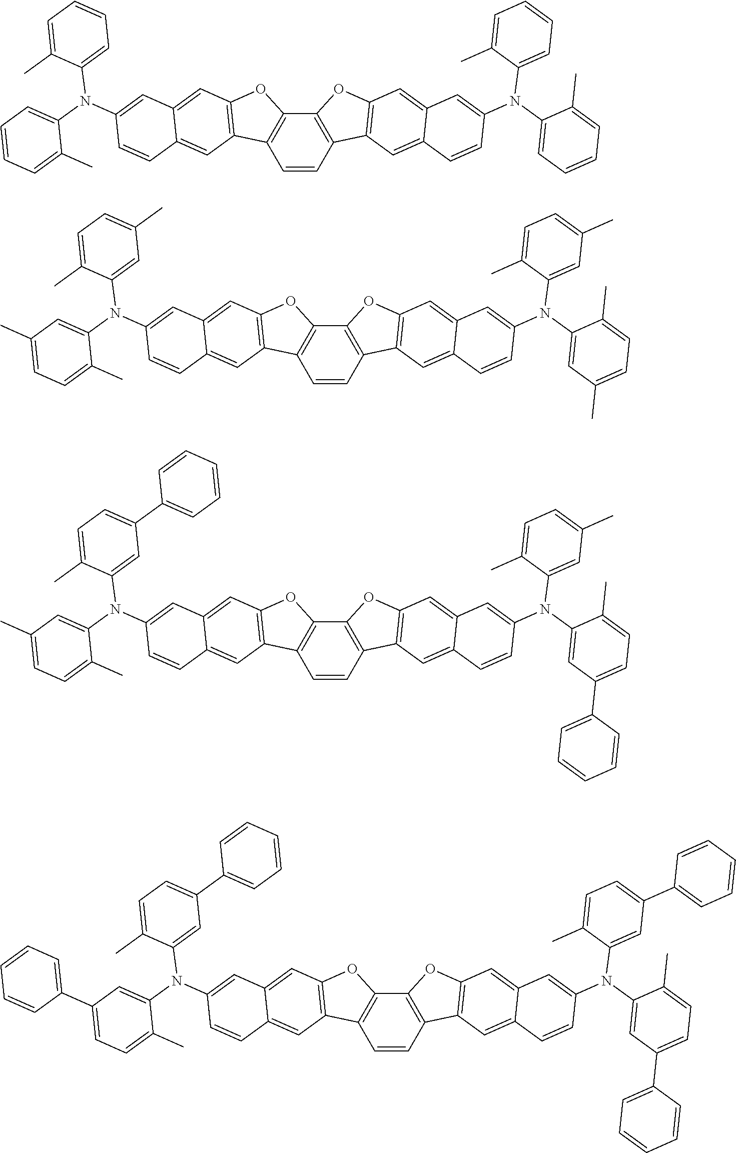

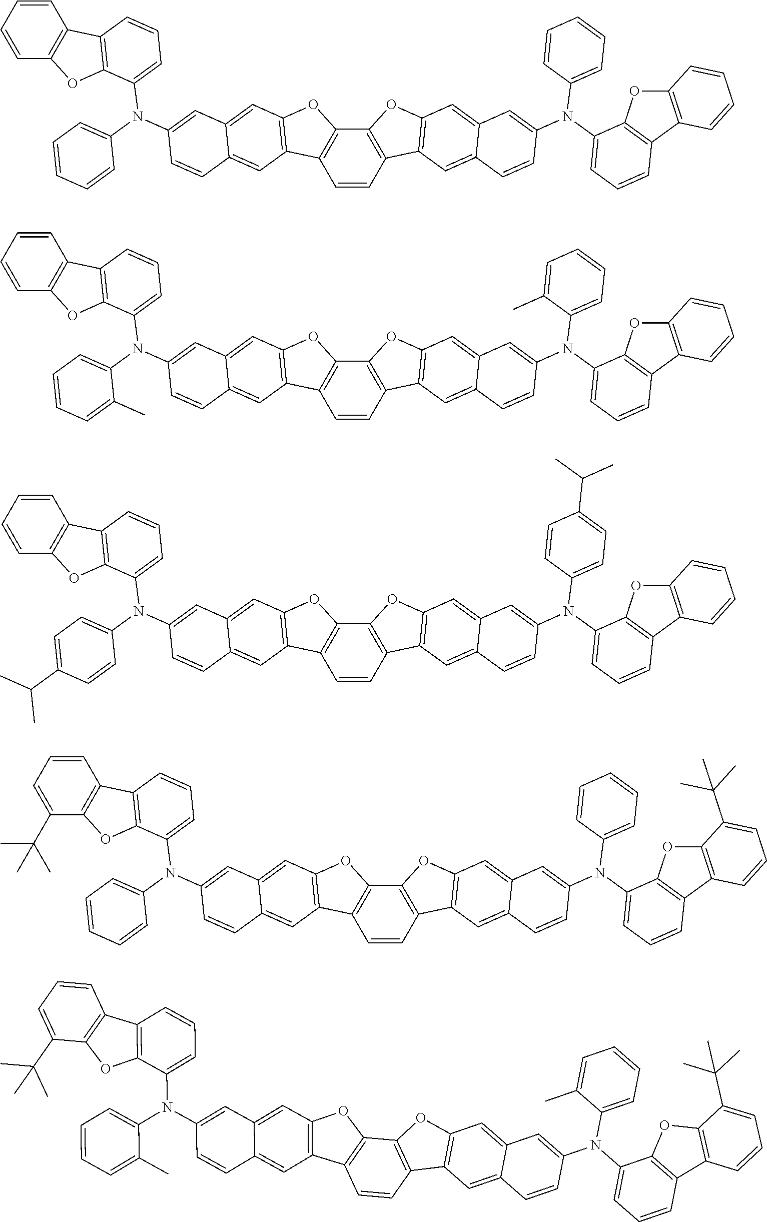

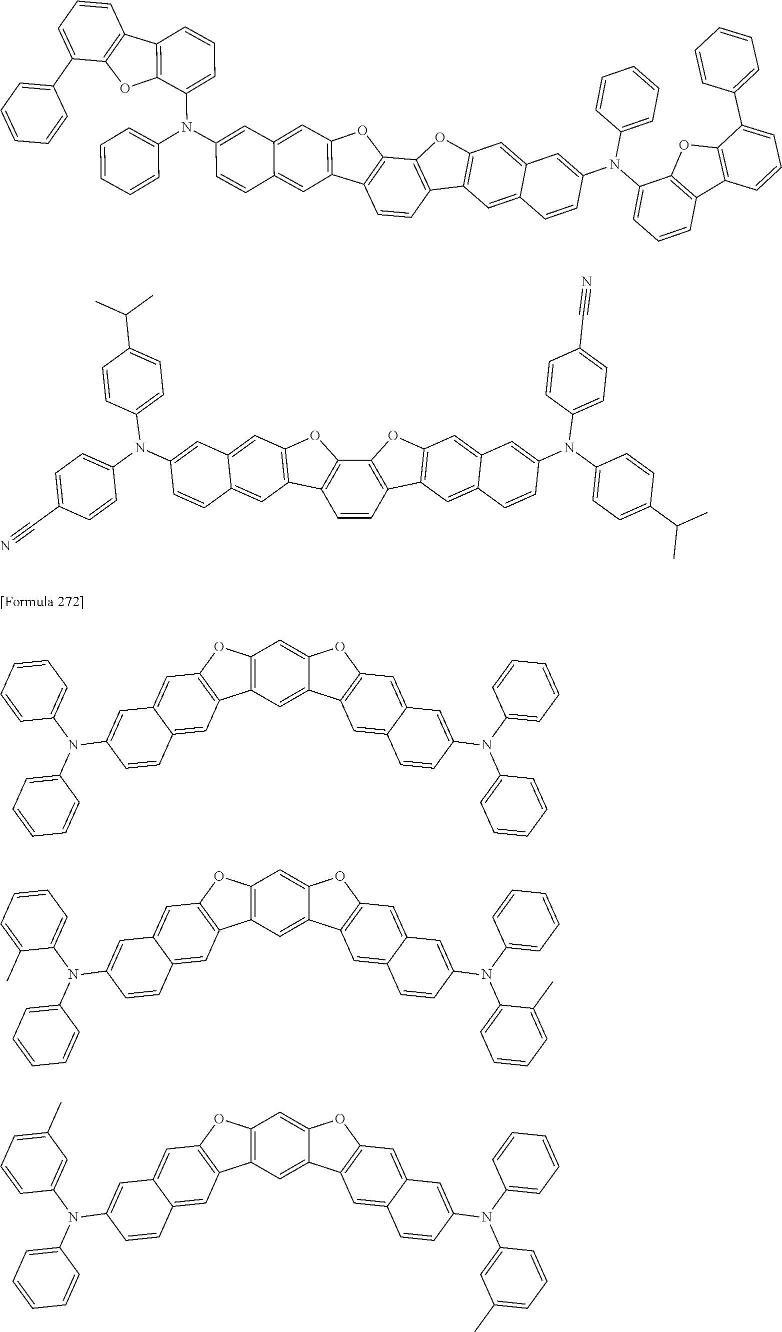

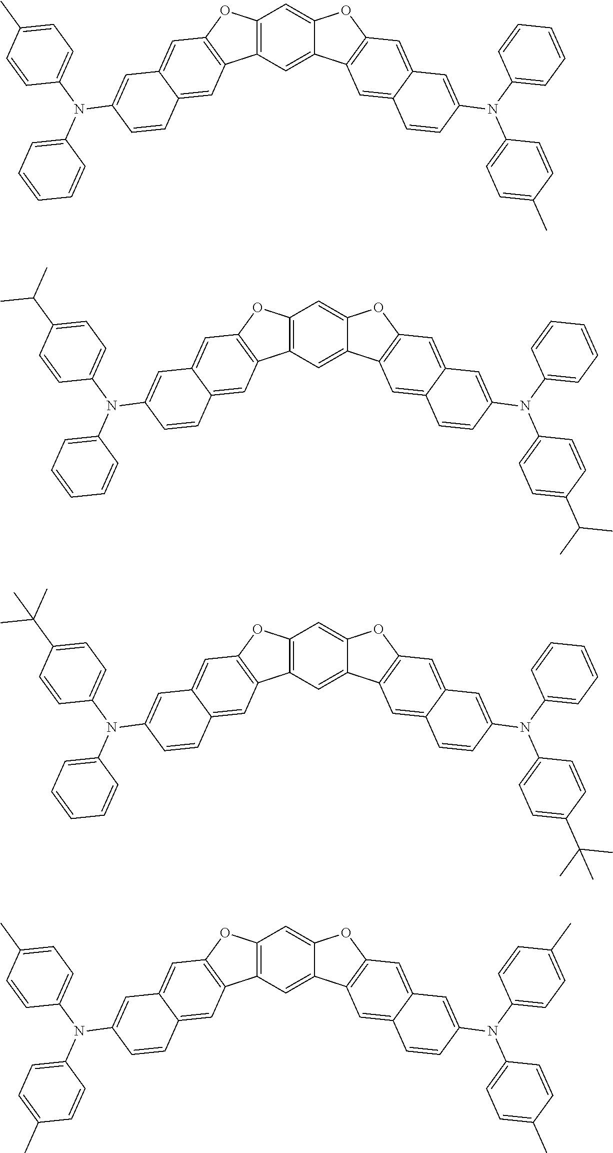

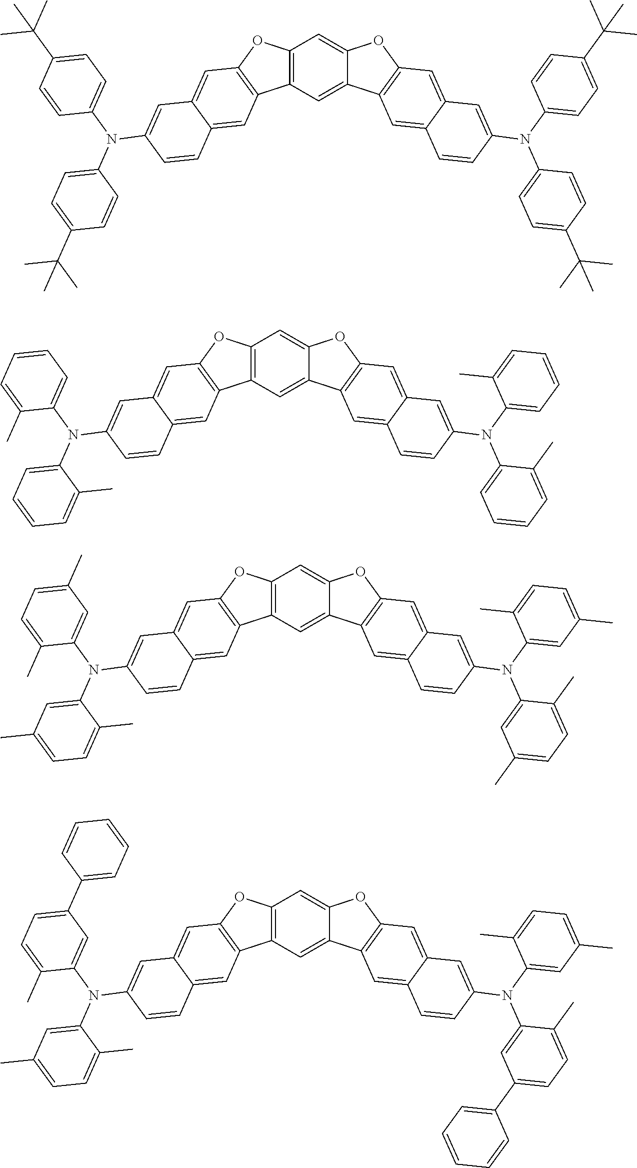

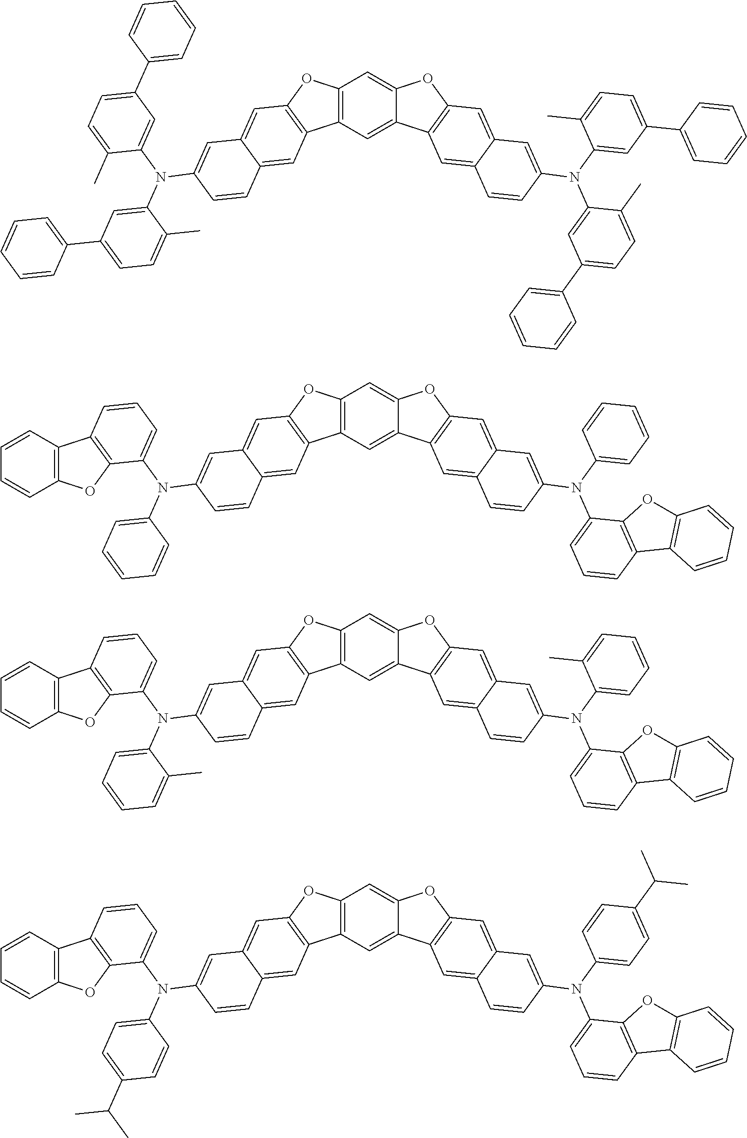 [SMILES: Cc1ccc(-c2ccccc2)cc1N(c1ccc2cc3c(cc2c1)oc1cc2oc4cc5cc(N(c6cc(-c7ccccc7)ccc6C)c6cc(-c7ccccc7)ccc6C)ccc5cc4c2cc13)c1cc(-c2ccccc2)ccc1C ; c1ccc(N(c2ccc3cc4c(cc3c2)oc2cc3oc5cc6cc(N(c7ccccc7)c7cccc8c7oc7ccccc78)ccc6cc5c3cc24)c2cccc3c2oc2ccccc23)cc1 ; Cc1ccccc1N(c1ccc2cc3c(cc2c1)oc1cc2oc4cc5cc(N(c6ccccc6C)c6cccc7c6oc6ccccc67)ccc5cc4c2cc13)c1cccc2c1oc1ccccc12 ; CC(C)c1ccc(N(c2ccc3cc4c(cc3c2)oc2cc3oc5cc6cc(N(c7ccc(C(C)C)cc7)c7cccc8c7oc7ccccc78)ccc6cc5c3cc24)c2cccc3c2oc2ccccc23)cc1]

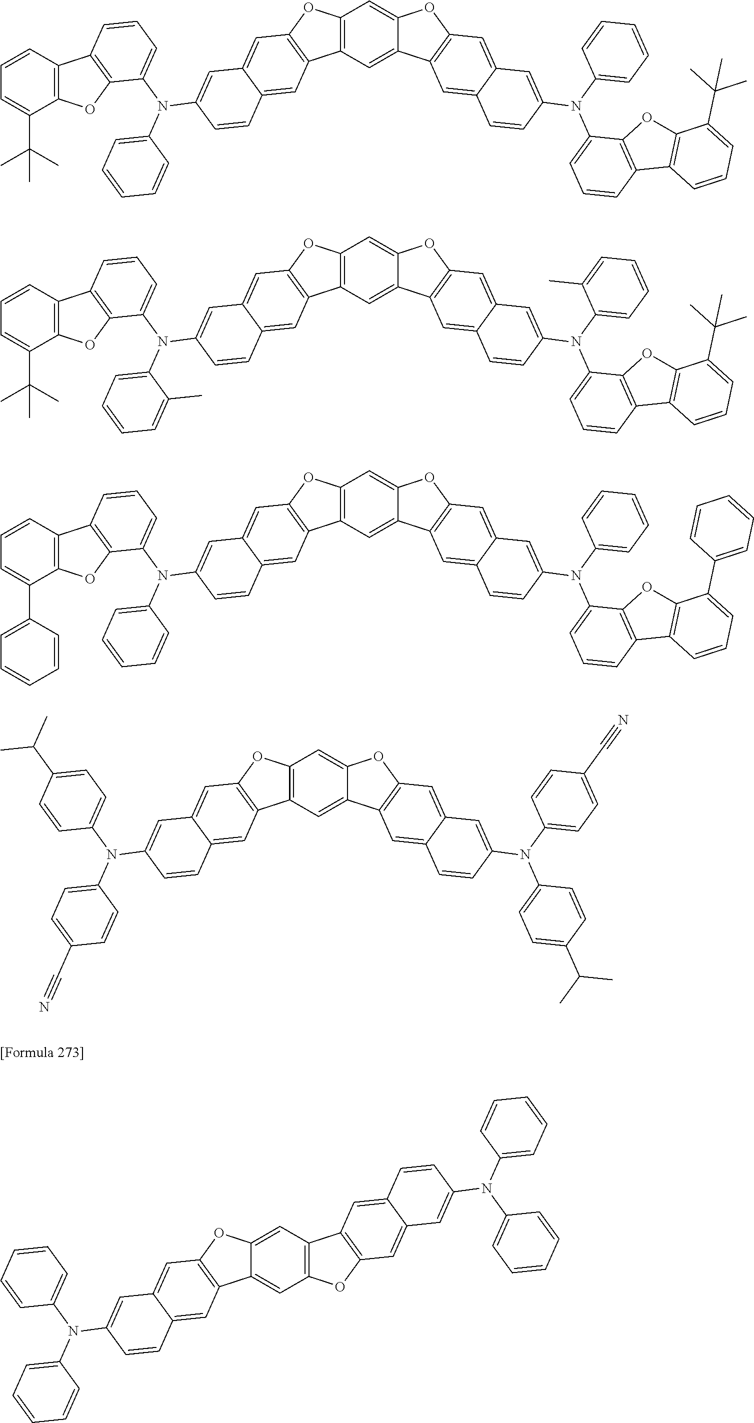

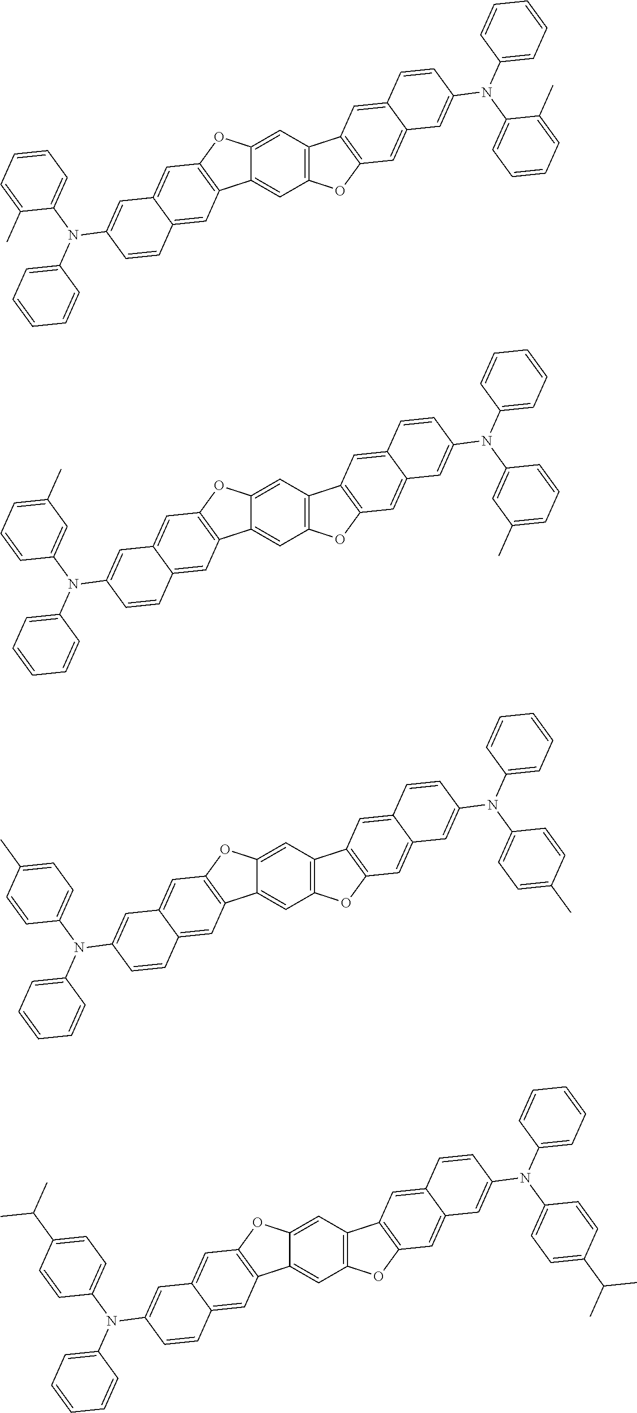

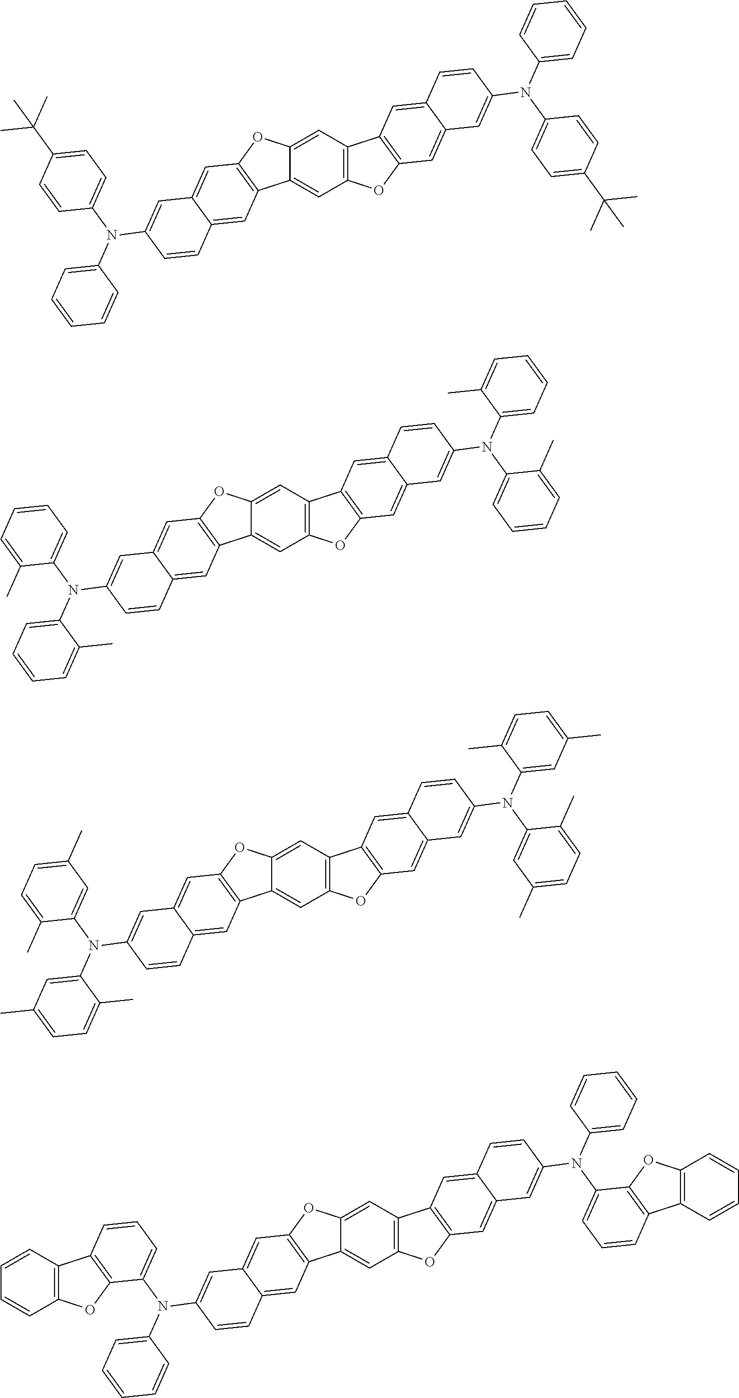

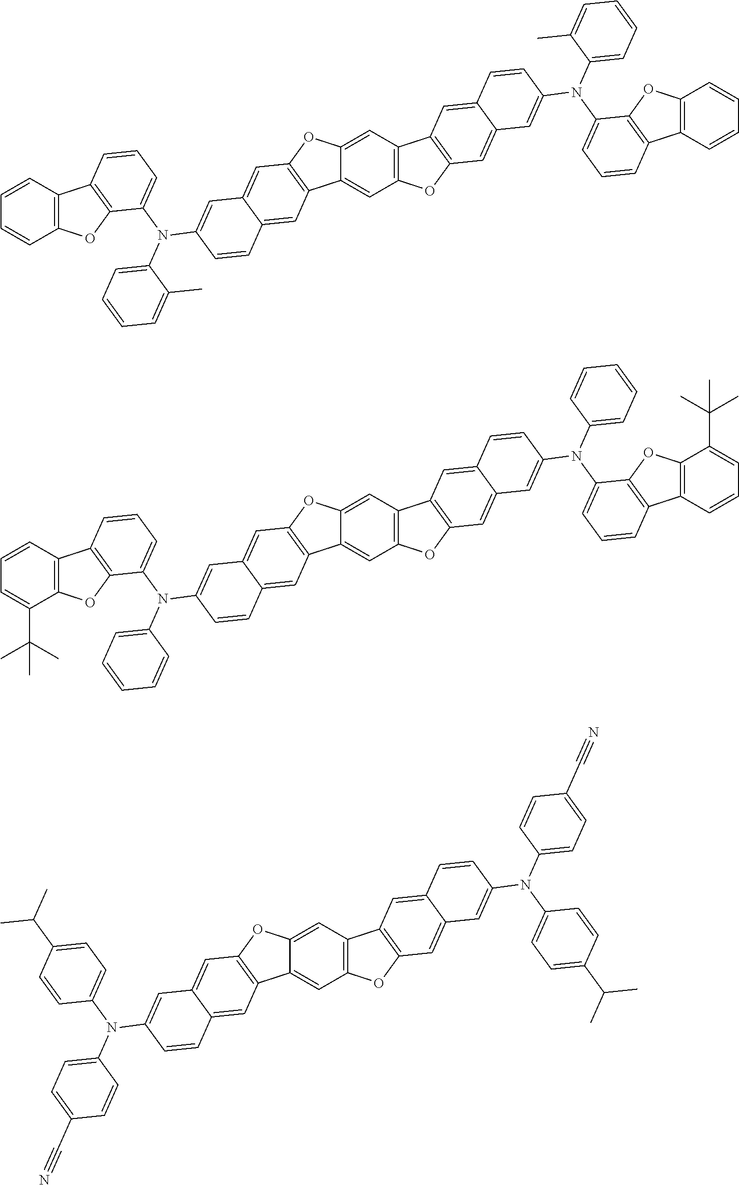

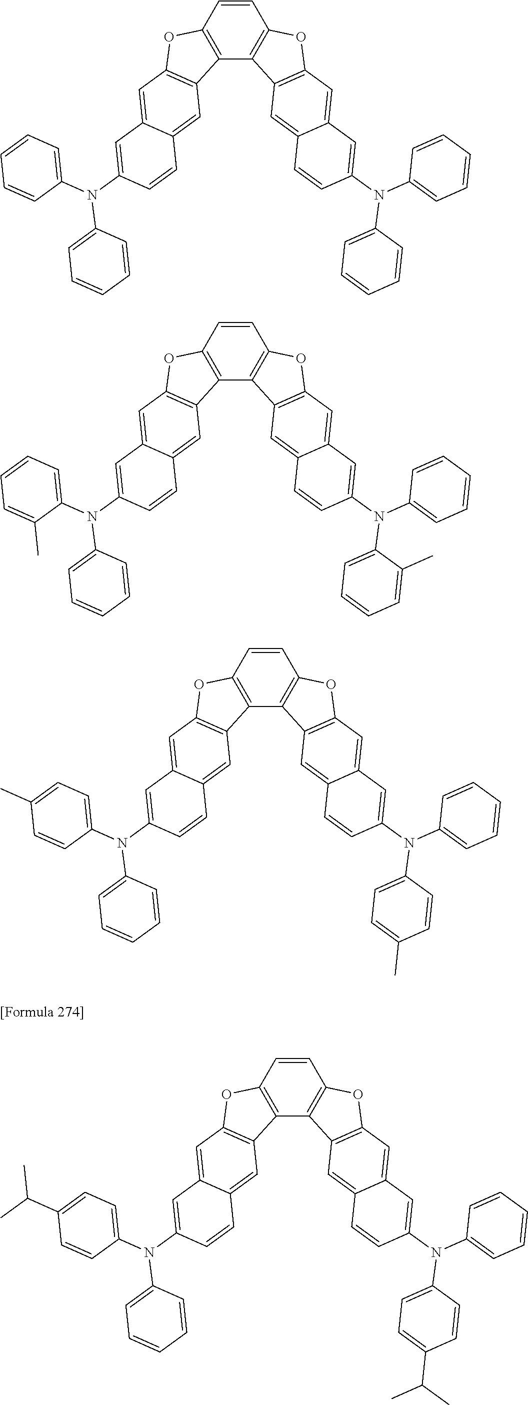 [SMILES: c1ccc(N(c2ccccc2)c2ccc3cc4c(cc3c2)oc2ccc3oc5cc6cc(N(c7ccccc7)c7ccccc7)ccc6cc5c3c24)cc1 ; Cc1ccccc1N(c1ccccc1)c1ccc2cc3c(cc2c1)oc1ccc2oc4cc5cc(N(c6ccccc6)c6ccccc6C)ccc5cc4c2c13 ; Cc1ccc(N(c2ccccc2)c2ccc3cc4c(cc3c2)oc2ccc3oc5cc6cc(N(c7ccccc7)c7ccc(C)cc7)ccc6cc5c3c24)cc1 ; CC(C)c1ccc(N(c2ccccc2)c2ccc3cc4c(cc3c2)oc2ccc3oc5cc6cc(N(c7ccccc7)c7ccc(C(C)C)cc7)ccc6cc5c3c24)cc1]

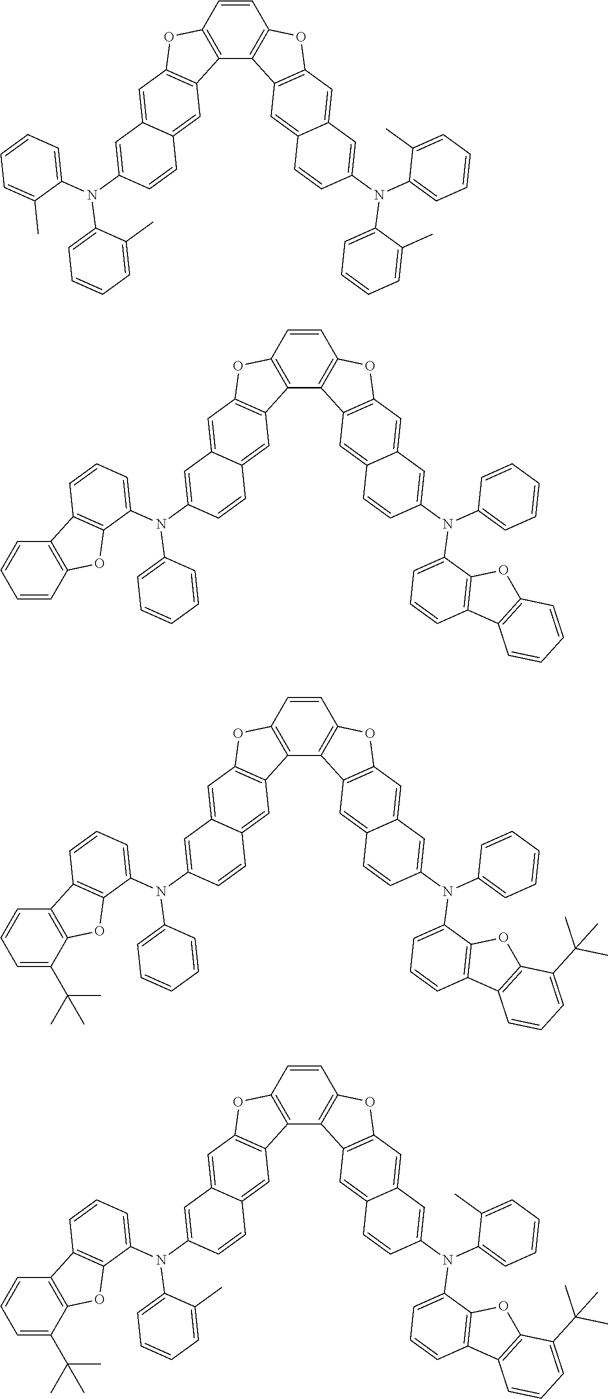 [SMILES: Cc1ccccc1N(c1ccc2cc3c(cc2c1)oc1ccc2oc4cc5cc(N(c6ccccc6C)c6ccccc6C)ccc5cc4c2c13)c1ccccc1C ; c1ccc(N(c2ccc3cc4c(cc3c2)oc2ccc3oc5cc6cc(N(c7ccccc7)c7cccc8c7oc7ccccc78)ccc6cc5c3c24)c2cccc3c2oc2ccccc23)cc1 ; CC(C)(C)c1cccc2c1oc1c(N(c3ccccc3)c3ccc4cc5c(cc4c3)oc3ccc4oc6cc7cc(N(c8ccccc8)c8cccc9c8oc8c(C(C)(C)C)cccc89)ccc7cc6c4c35)cccc12 ; Cc1ccccc1N(c1ccc2cc3c(cc2c1)oc1ccc2oc4cc5cc(N(c6ccccc6C)c6cccc7c6oc6c(C(C)(C)C)cccc67)ccc5cc4c2c13)c1cccc2c1oc1c(C(C)(C)C)cccc12]

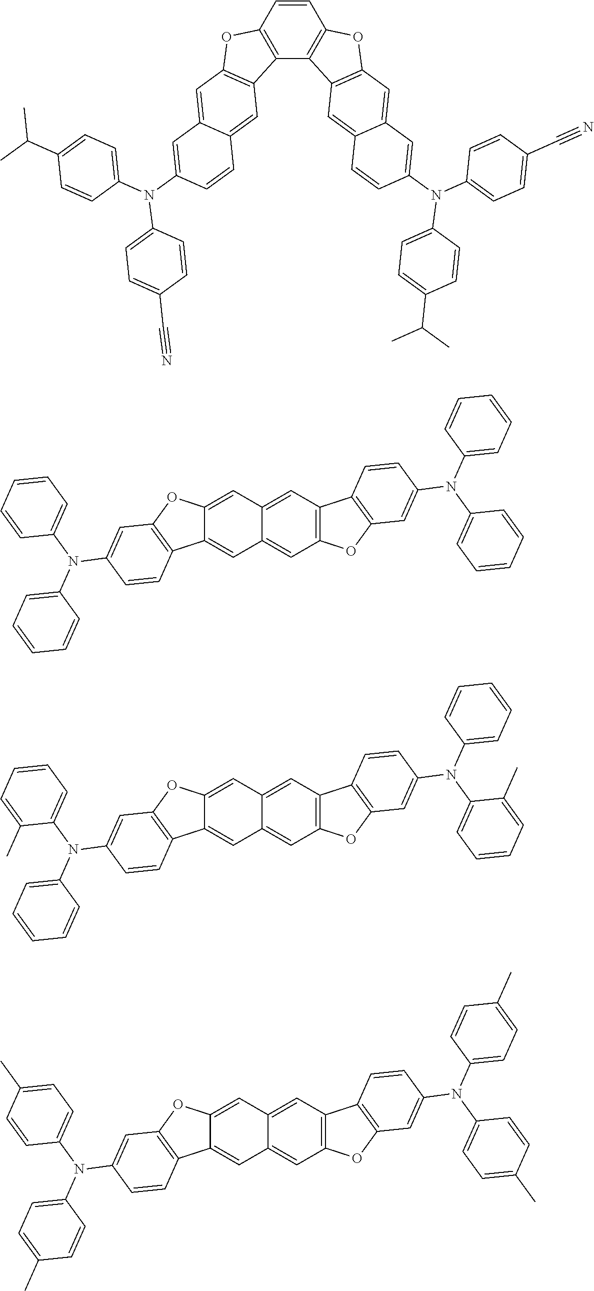

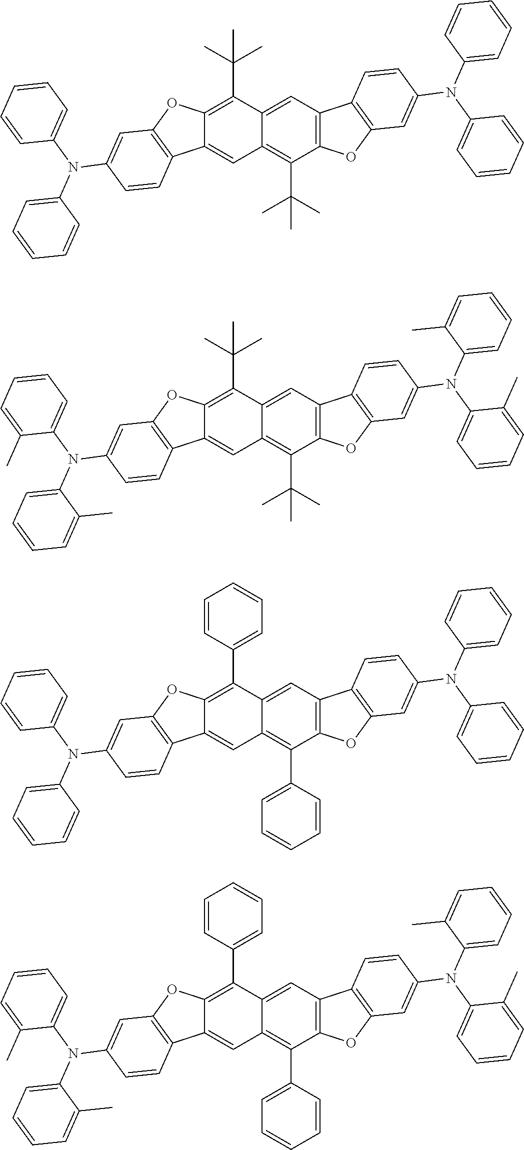 [SMILES: CC(C)(C)c1c2cc3c(oc4cc(N(c5ccccc5)c5ccccc5)ccc43)c(C(C)(C)C)c2cc2c1oc1cc(N(c3ccccc3)c3ccccc3)ccc12 ; Cc1ccccc1N(c1ccc2c(c1)oc1c(C(C)(C)C)c3cc4c(oc5cc(N(c6ccccc6C)c6ccccc6C)ccc54)c(C(C)(C)C)c3cc12)c1ccccc1C ; c1ccc(-c2c3cc4c(oc5cc(N(c6ccccc6)c6ccccc6)ccc54)c(-c4ccccc4)c3cc3c2oc2cc(N(c4ccccc4)c4ccccc4)ccc23)cc1 ; Cc1ccccc1N(c1ccc2c(c1)oc1c(-c3ccccc3)c3cc4c(oc5cc(N(c6ccccc6C)c6ccccc6C)ccc54)c(-c4ccccc4)c3cc12)c1ccccc1C]

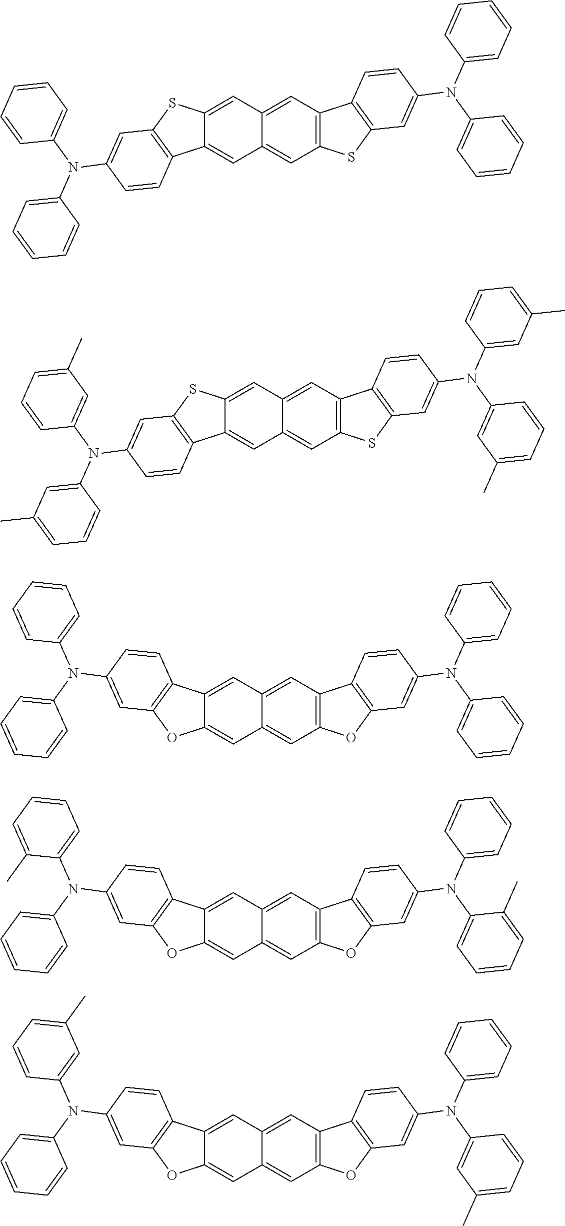

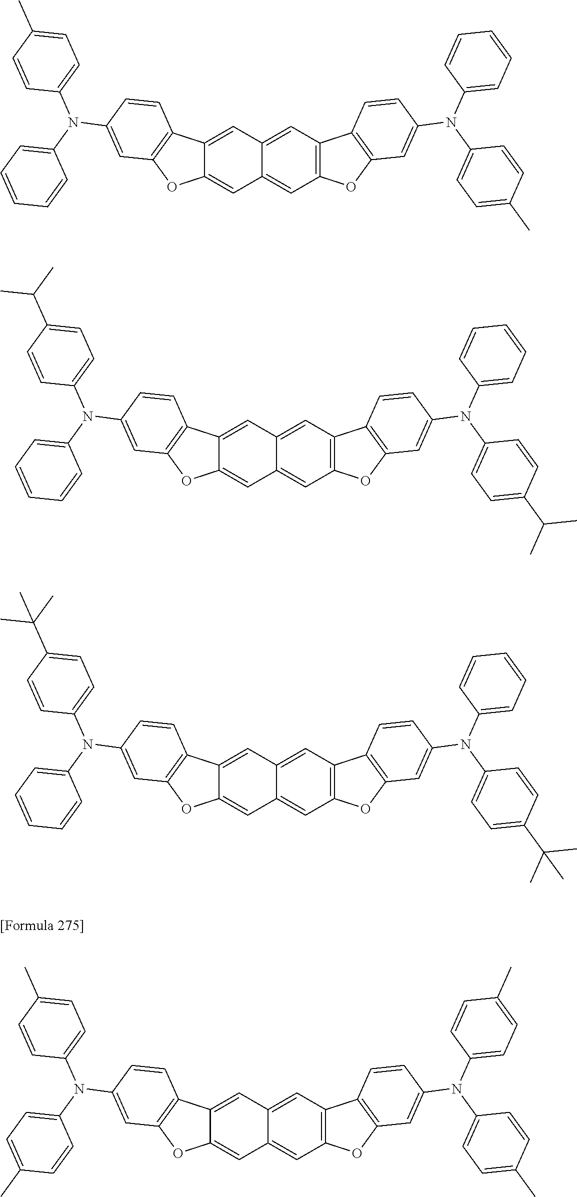

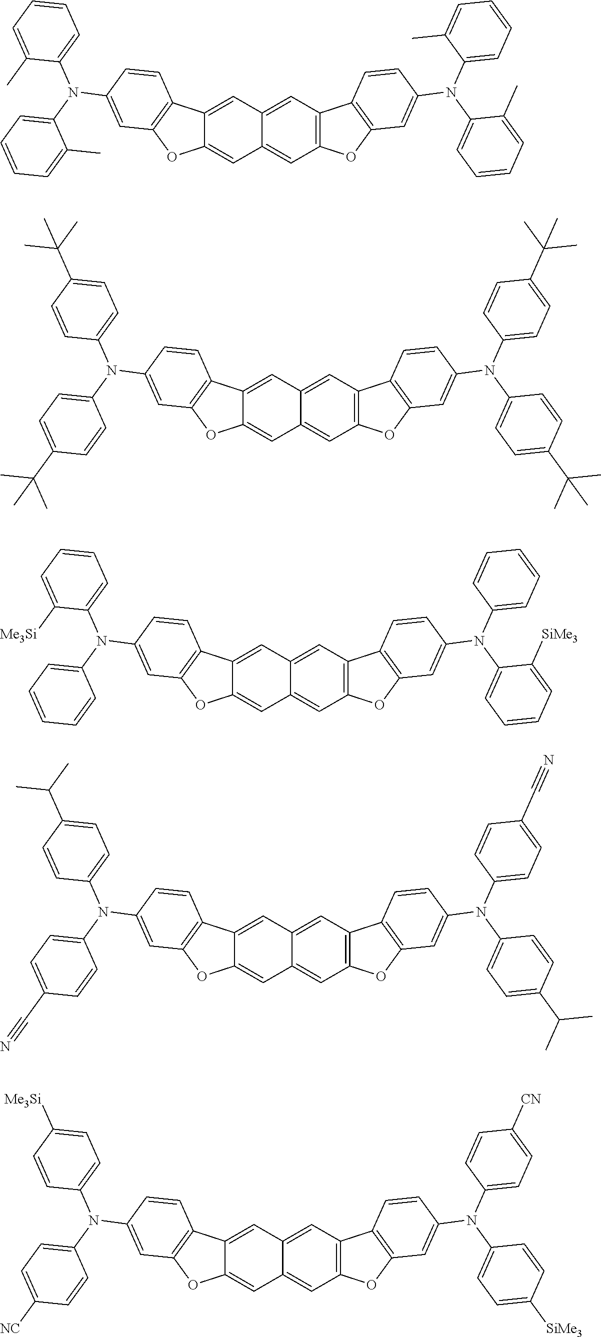

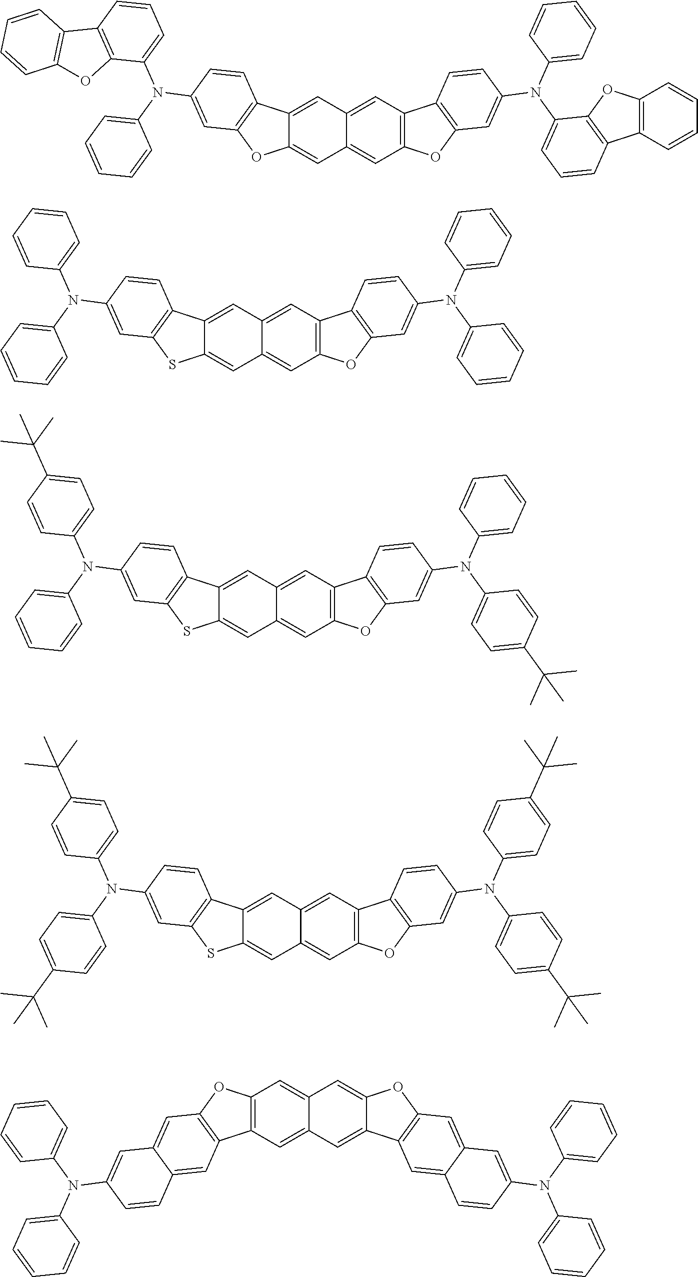

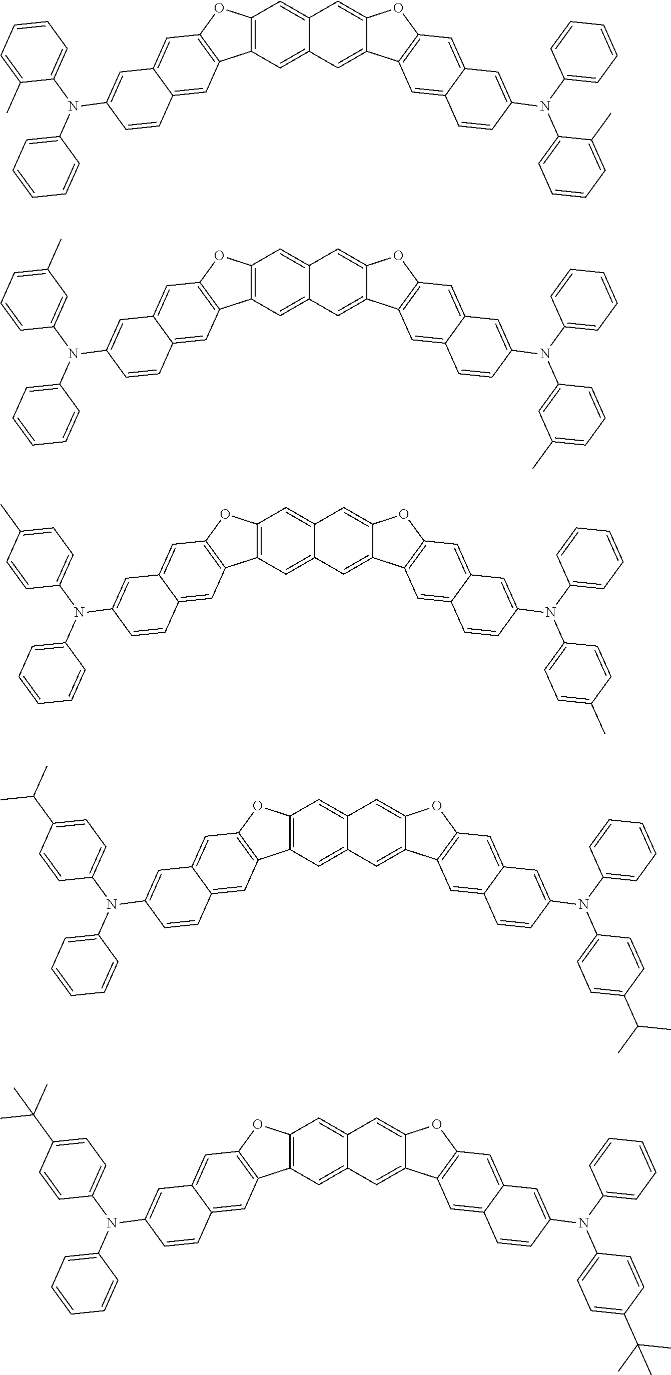

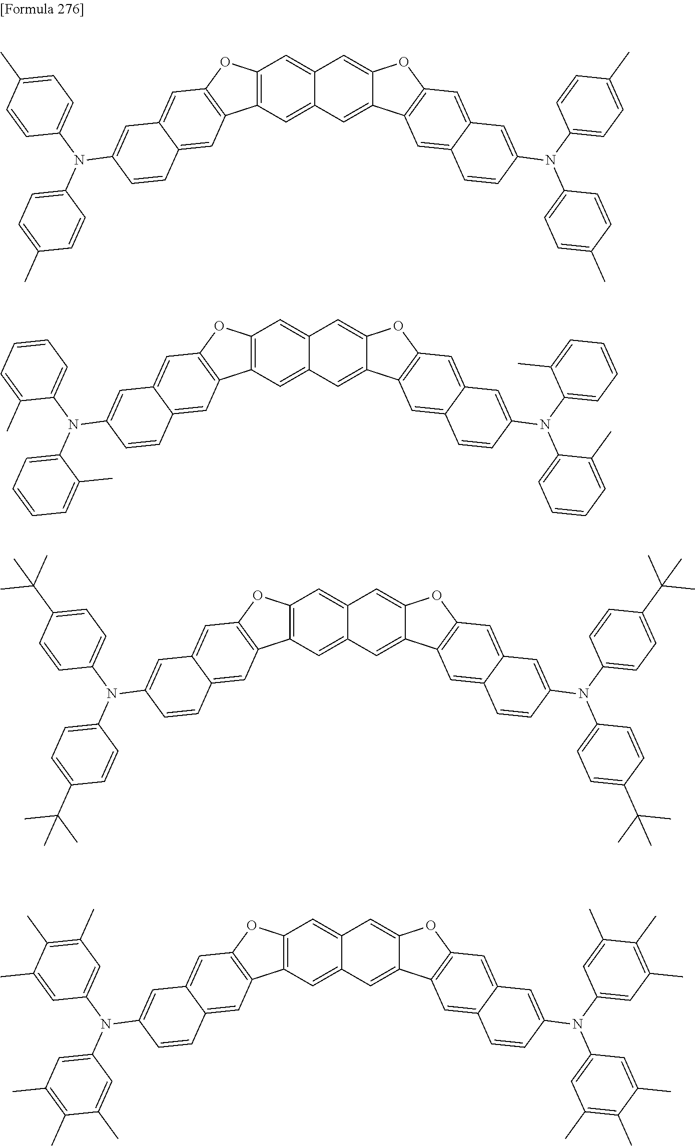

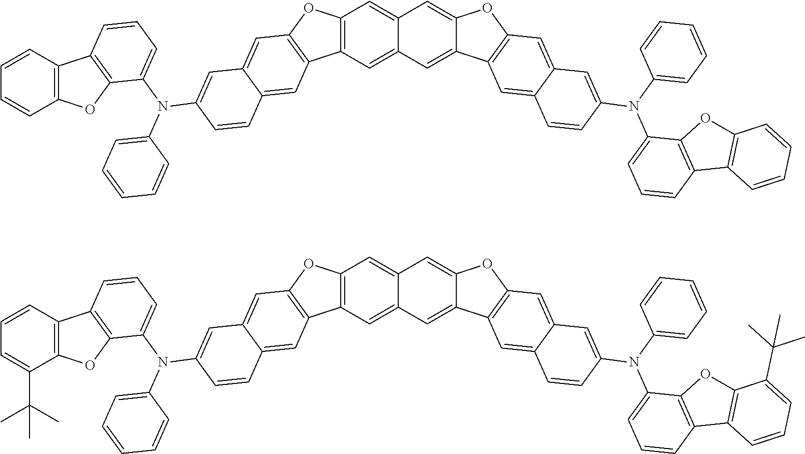 [SMILES: c1ccc(N(c2ccc3cc4c(cc3c2)oc2cc3cc5oc6cc7cc(N(c8ccccc8)c8cccc9c8oc8ccccc89)ccc7cc6c5cc3cc24)c2cccc3c2oc2ccccc23)cc1 ; CC(C)(C)c1cccc2c1oc1c(N(c3ccccc3)c3ccc4cc5c(cc4c3)oc3cc4cc6oc7cc8cc(N(c9ccccc9)c9cccc%10c9oc9c(C(C)(C)C)cccc9%10)ccc8cc7c6cc4cc35)cccc12]

Compound Represented by Formula (8)

A compound represented by the formula (8) is described below.

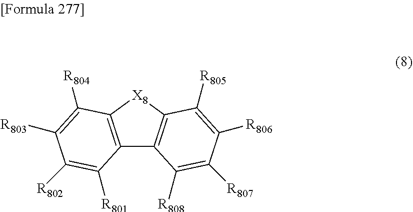

In the formula (8),
at least one combination of R₈₀₁ and R₈₀₂, R₈₀₂ and R₈₀₃, or R₈₀₃ and R₈₀₄ are mutually bonded to form a divalent group represented by the following formula (82), and
at least one combination of R₈₀₅ and R₈₀₆, R₈₀₆ and R₈₀₇, or R₈₀₇ and R₈₀₈ are mutually bonded to form a divalent group represented by the following formula (83).

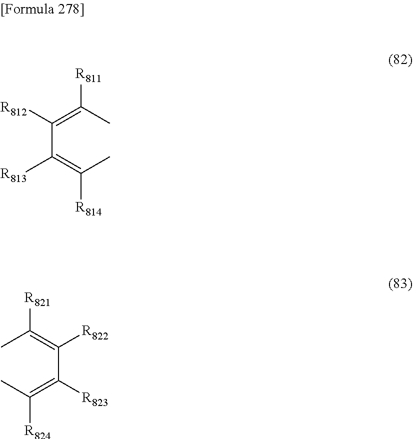

At least one of R₈₀₁ to R₈₀₄ that do not form the divalent group represented by the formula (82) and R₈₁₁ to R₈₁₄ represents a monovalent group represented by the following formula (84),
at least one of R₈₀₅ to R₈₀₈ that do not form the divalent group represented by the formula (83) and R₈₂₁ to R₈₂₄ represents a monovalent group represented by the following formula (84),
X₈ represents an oxygen atom, a sulfur atom, or NR₈₀₉, and
R₈₀₁ to R₈₀₈ that do not form the divalent groups represented by the formula (82) and the formula (83) and are not monovalent groups represented by the formula (84), R₈₁₁ to R₈₁₄ and R₈₂₁ to R₈₂₄ that are not monovalent groups represented by the formula (84), and R₈₀₉ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

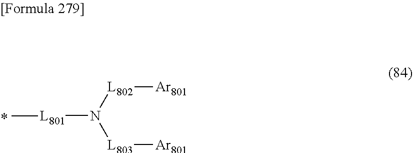

In the formula (84),
Ar₈₀₁ and Ar₈₀₂ each independently represent a substituted or unsubstituted aryl group having 6 to 50 ring carbon atoms, or a substituted or unsubstituted heterocyclic group having 5 to 50 ring atoms,
L₈₀₁ to L₈₀₃ each independently represent a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms, and
* in the formula (84) represents a bonding position to the cyclic structure represented by the formula (8) or the group represented by the formula (82) or (83).

In the formula (8), the positions for the divalent group represented by the formula (82) and the divalent group represented by the formula (83) to be formed are not specifically limited but the divalent groups may be formed at any possible positions on R₈₀₁ to R₈₀₈.

In an exemplary embodiment, a compound represented by the formula (8) is represented by one of the following formulae (81-1) to (81-6).

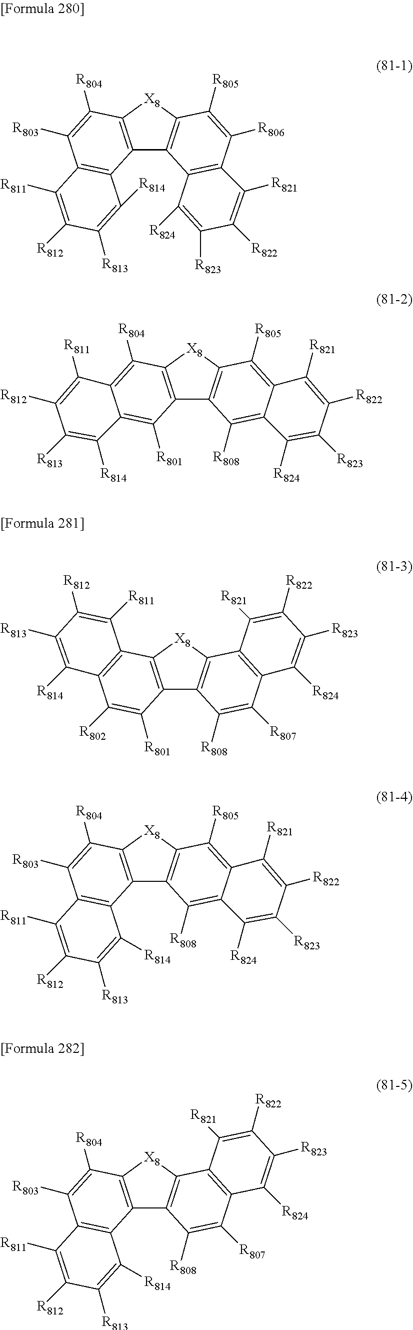

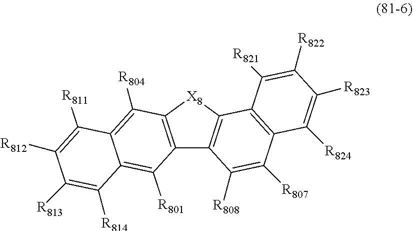

In the formula (81-1) to (81-6),
X₈ represents the same as X₈ in the formula (8),
at least two of R₈₀₁ to R₈₂₄ are monovalent groups represented by the formula (84), and
R₈₀₁ to R₈₂₄ that are not monovalent groups represented by the formula (84) each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, a compound represented by the formula (8) is represented by one of the following formulae (81-7) to (81-18).

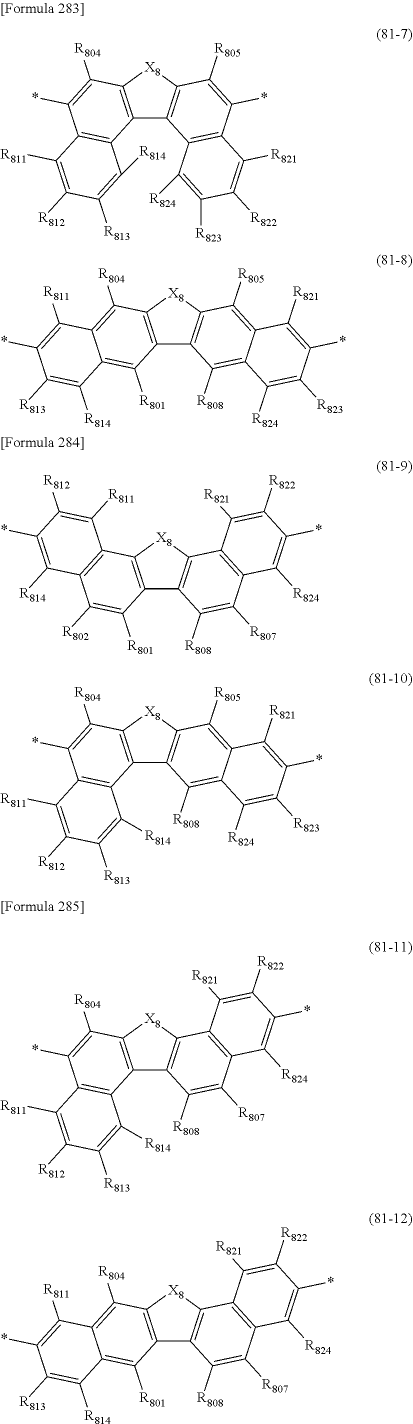

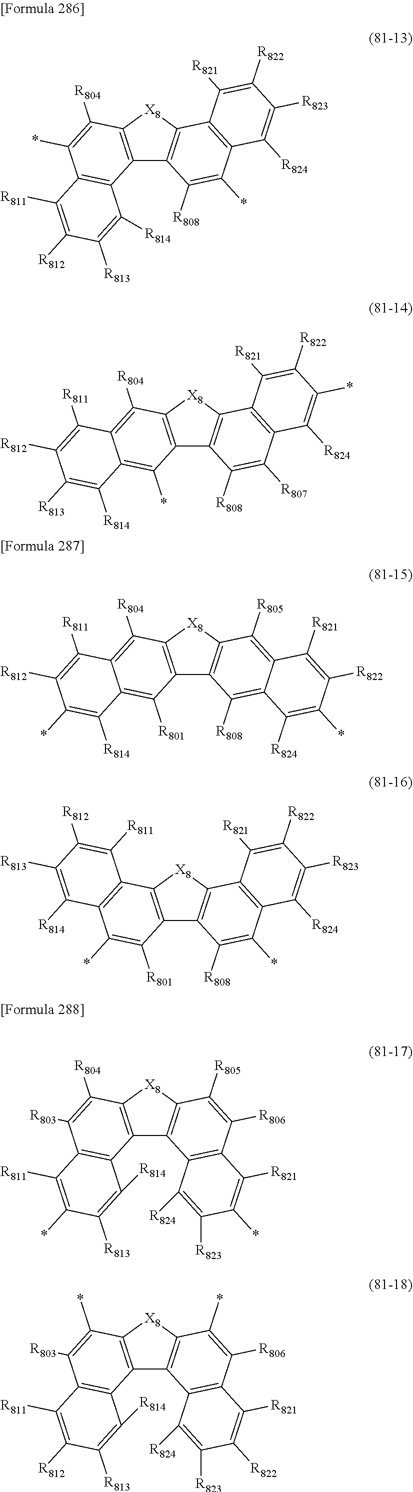

In the formula (81-7) to (81-18),
X₈ represents the same as X₈ in the formula (8),
* represents a single bond bonded to a monovalent group represented by the formula (84), and
R₈₀₁ to R₈₂₄ represent the same as R₈₀₁ to R₈₂₄ that are not monovalent groups represented by the formula (84), respectively, in the formulae (81-1) to (81-6).
R₈₀₁ to R₈₀₈ that do not form the divalent groups represented by the formula (82) and the formula (83) and are not monovalent groups represented by the formula (84) and R₈₁₁ to R₈₁₄ and R₈₂₁ to R₈₂₄ that are not monovalent groups represented by the formula (84) preferably each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

A monovalent group represented by the formula (84) is preferably represented by the following formula (85) or (86).

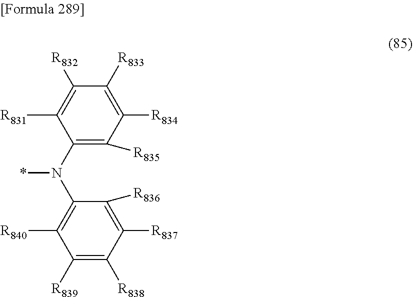

In the formula (85),
R₈₃₁ to R₈₄₀ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms, and
* in the formula (85) represents the same as * in the formula (84).

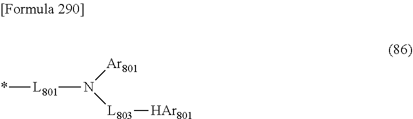

In the formula (86), Ar₈₀₁, L₈₀₁, and L₈₀₃ represent the same as Ar₈₀₁, L₈₀₁, and L₈₀₃, respectively, in the formula (84), and HAr₈₀₁ represents a structure represented by the following formula (87).

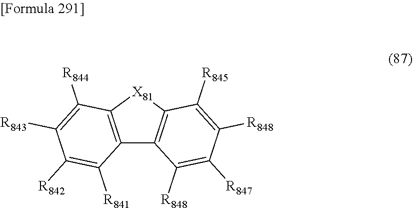

In the formula (87),
X₈₁ represents an oxygen atom or a sulfur atom,
any one of R₈₄₁ to R₈₄₈ represents a single bond bonded to L₈₀₃, and
R₈₄₁ to R₈₄₈ that do not represent a single bond each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of a compound represented by the formula (8) include compounds described in International Publication No. WO 2014/104144 and the following compounds.

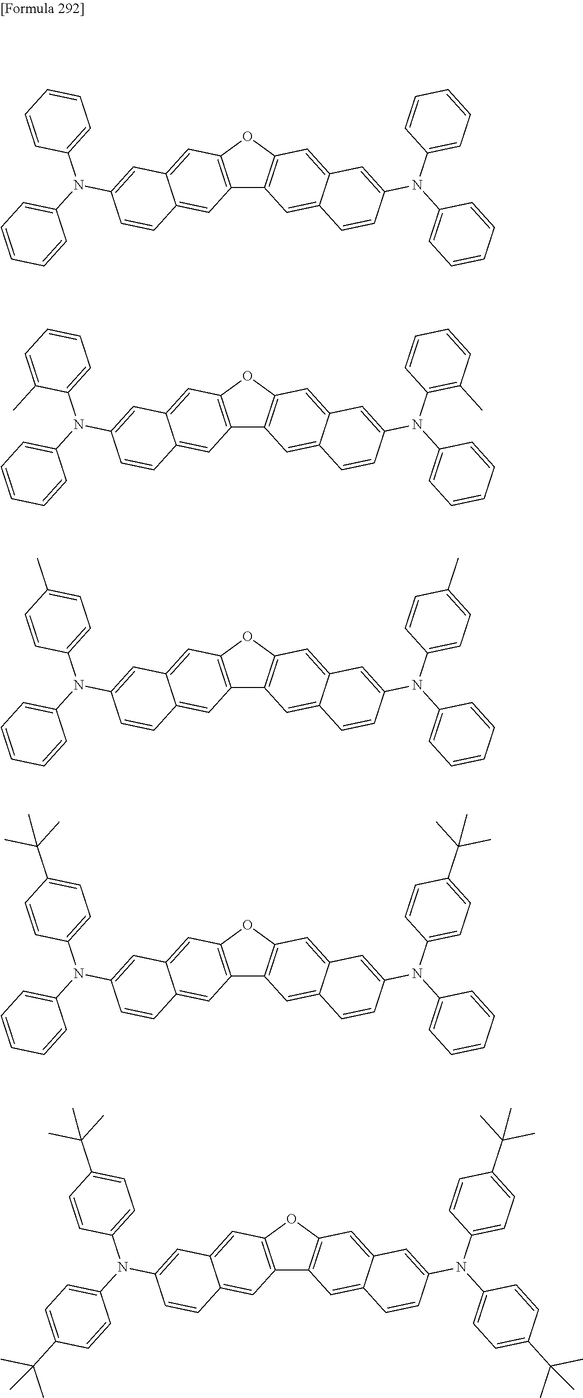 [SMILES: c1ccc(N(c2ccccc2)c2ccc3cc4c(cc3c2)oc2cc3cc(N(c5ccccc5)c5ccccc5)ccc3cc24)cc1 ; Cc1ccccc1N(c1ccccc1)c1ccc2cc3c(cc2c1)oc1cc2cc(N(c4ccccc4)c4ccccc4C)ccc2cc13 ; Cc1ccc(N(c2ccccc2)c2ccc3cc4c(cc3c2)oc2cc3cc(N(c5ccccc5)c5ccc(C)cc5)ccc3cc24)cc1 ; CC(C)(C)c1ccc(N(c2ccccc2)c2ccc3cc4c(cc3c2)oc2cc3cc(N(c5ccccc5)c5ccc(C(C)(C)C)cc5)ccc3cc24)cc1 ; CC(C)(C)c1ccc(N(c2ccc(C(C)(C)C)cc2)c2ccc3cc4c(cc3c2)oc2cc3cc(N(c5ccc(C(C)(C)C)cc5)c5ccc(C(C)(C)C)cc5)ccc3cc24)cc1]

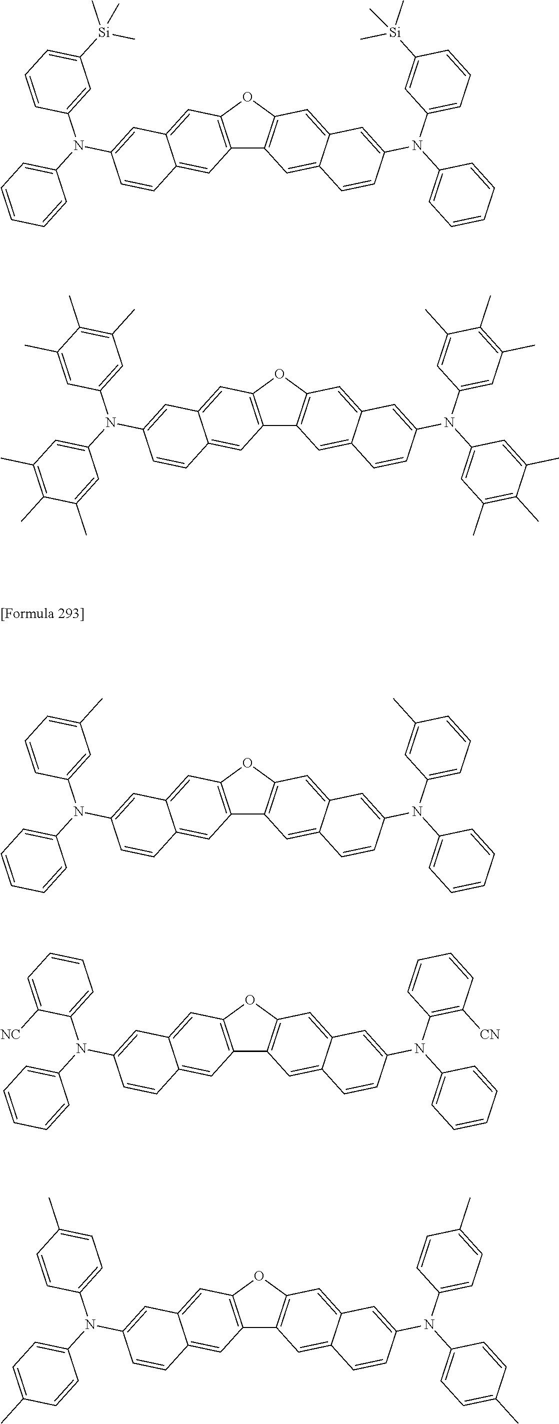

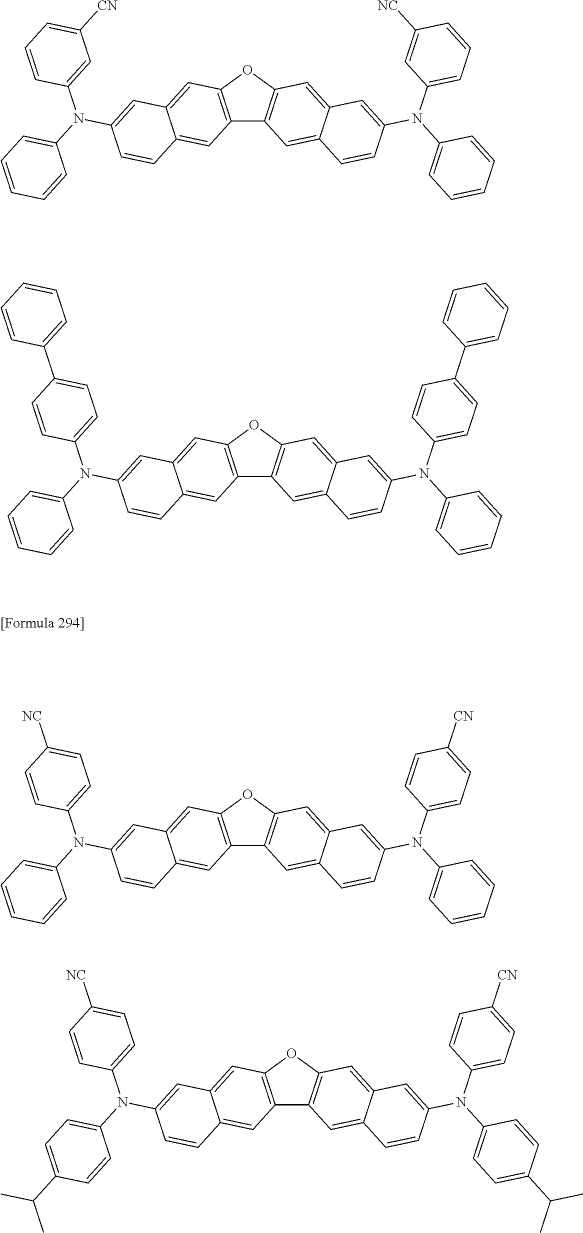 [SMILES: N#Cc1cccc(N(c2ccccc2)c2ccc3cc4c(cc3c2)oc2cc3cc(N(c5ccccc5)c5cccc(C#N)c5)ccc3cc24)c1 ; c1ccc(-c2ccc(N(c3ccccc3)c3ccc4cc5c(cc4c3)oc3cc4cc(N(c6ccccc6)c6ccc(-c7ccccc7)cc6)ccc4cc35)cc2)cc1 ; N#Cc1ccc(N(c2ccccc2)c2ccc3cc4c(cc3c2)oc2cc3cc(N(c5ccccc5)c5ccc(C#N)cc5)ccc3cc24)cc1 ; CC(C)c1ccc(N(c2ccc(C#N)cc2)c2ccc3cc4c(cc3c2)oc2cc3cc(N(c5ccc(C#N)cc5)c5ccc(C(C)C)cc5)ccc3cc24)cc1]

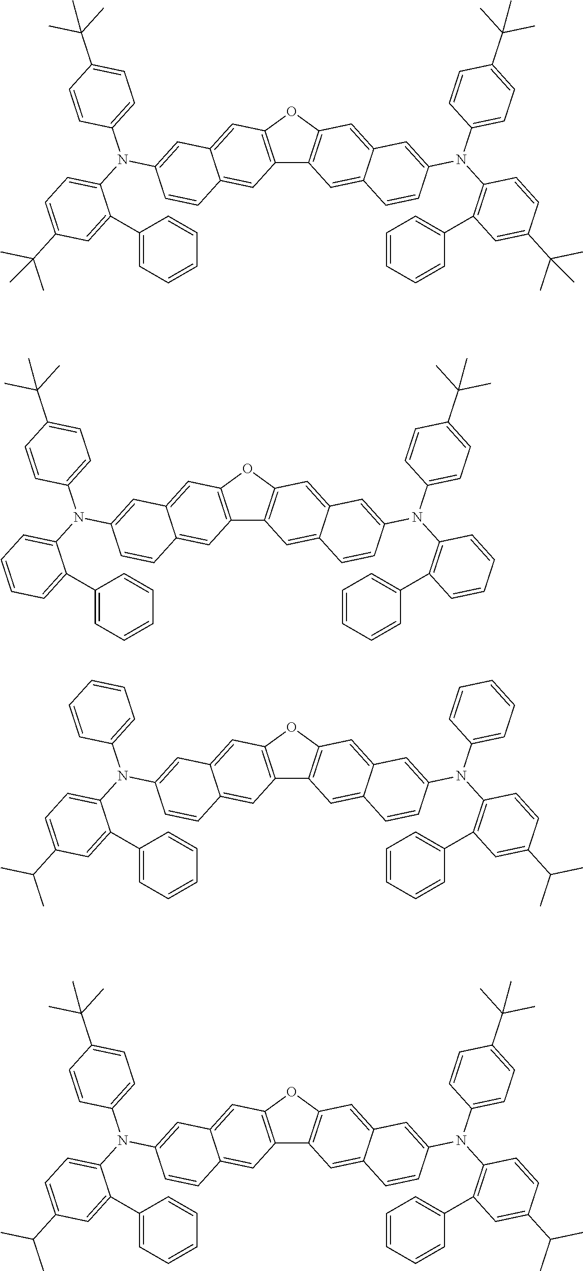

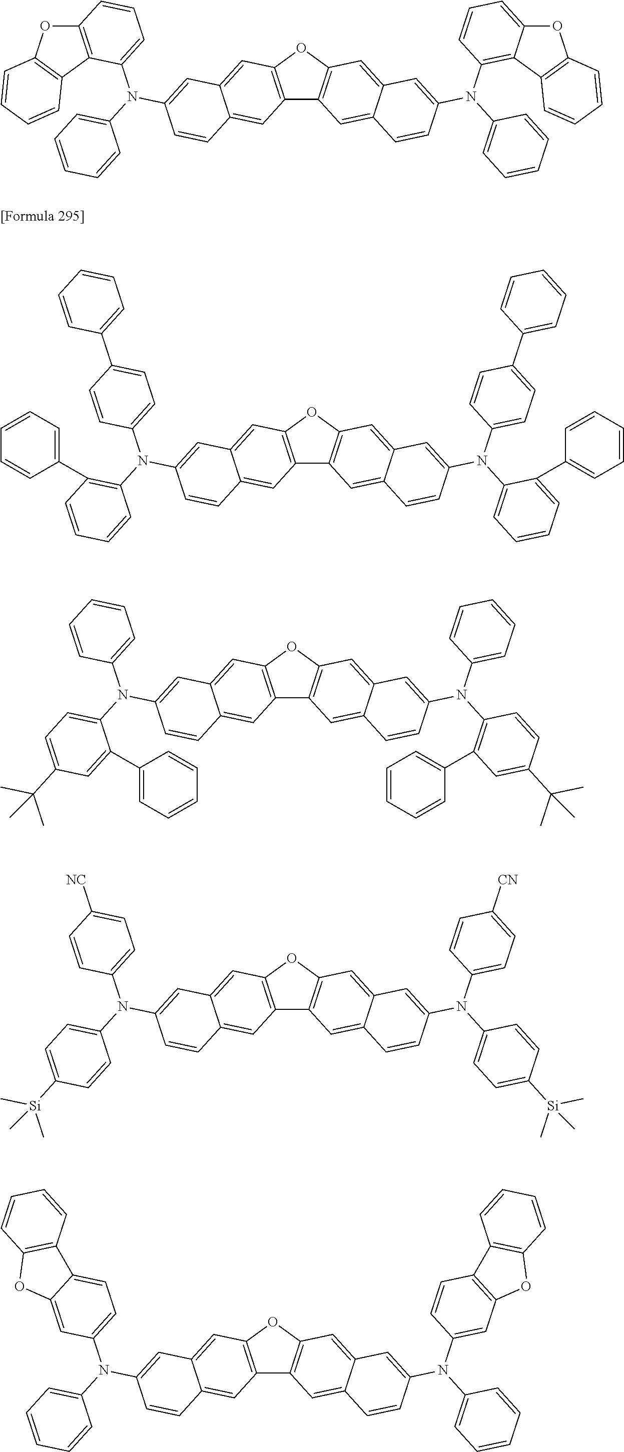

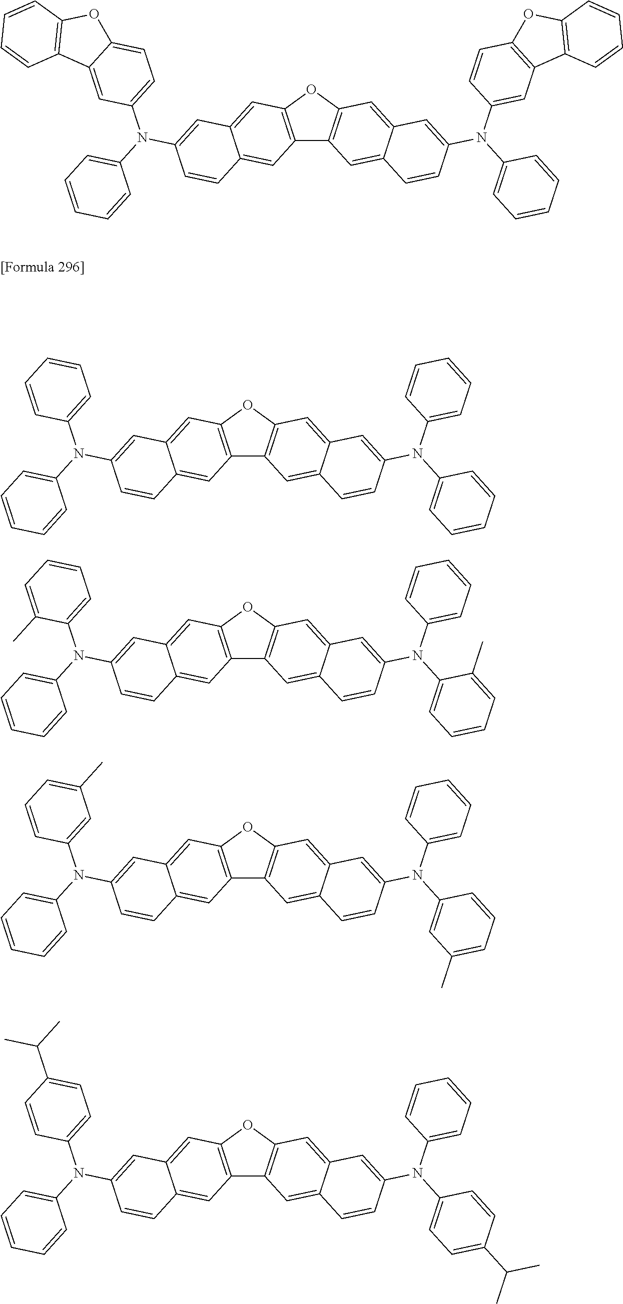

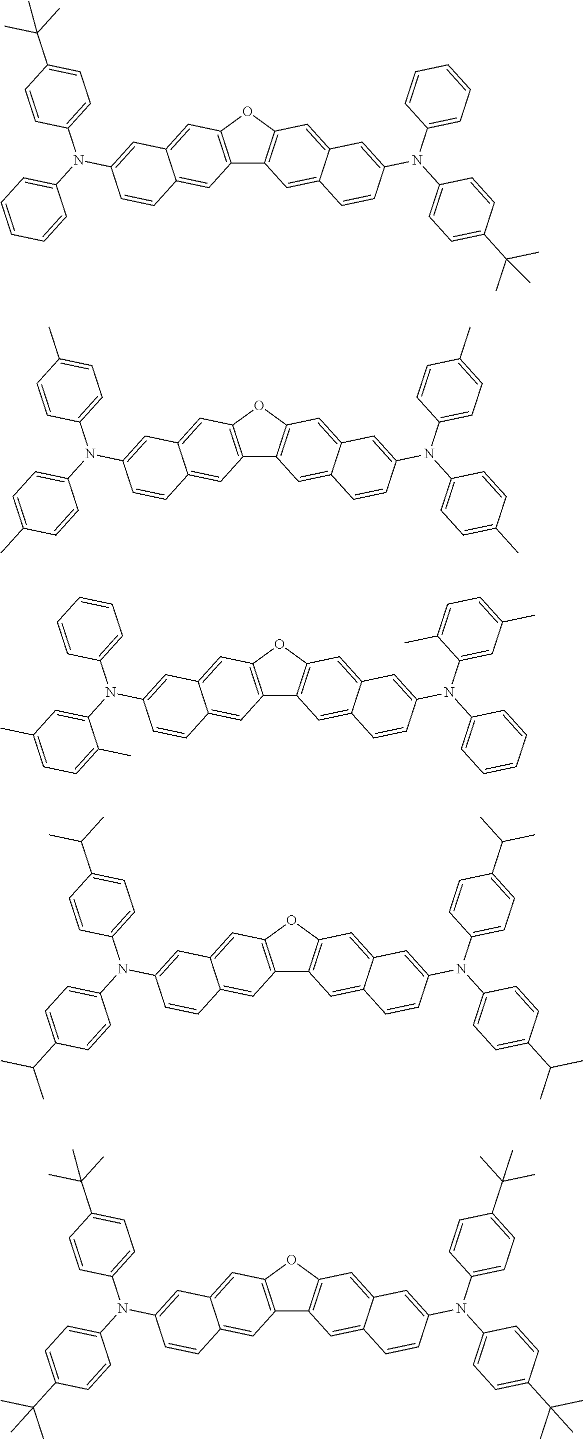

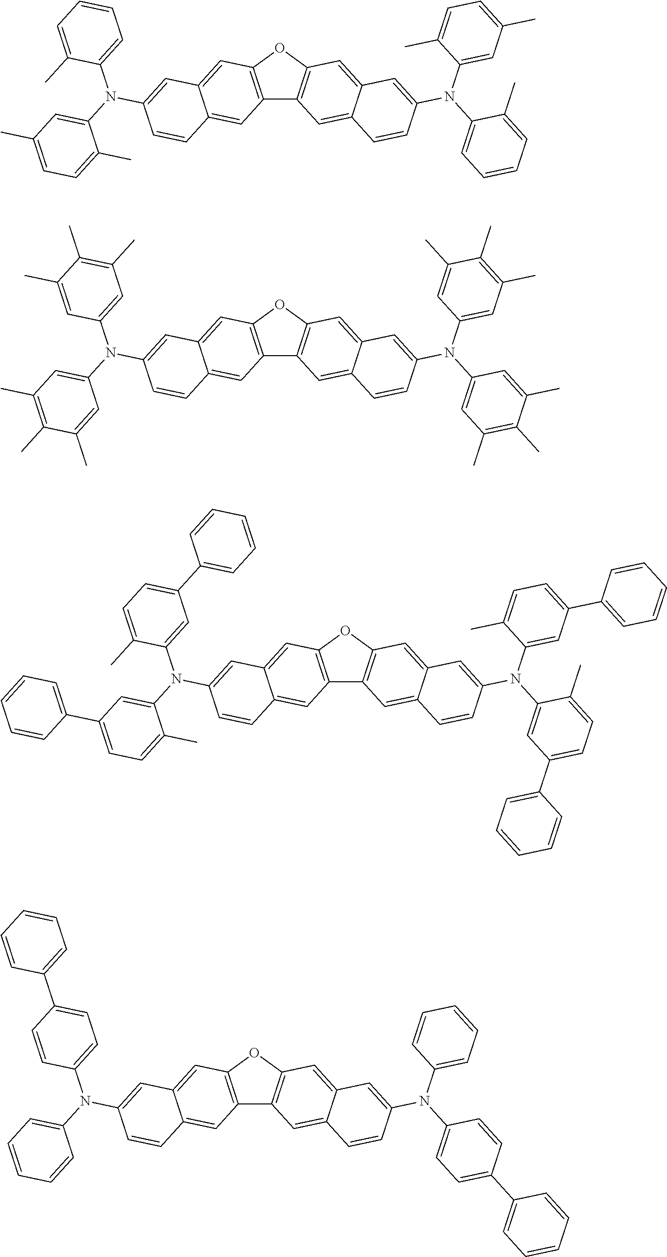

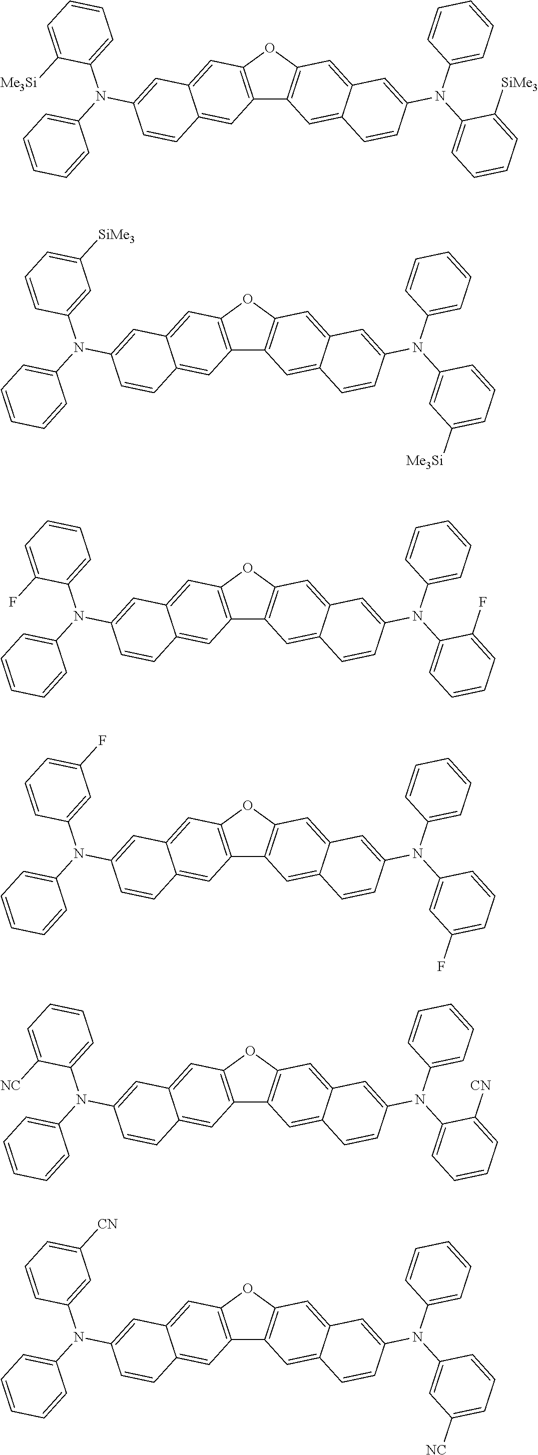

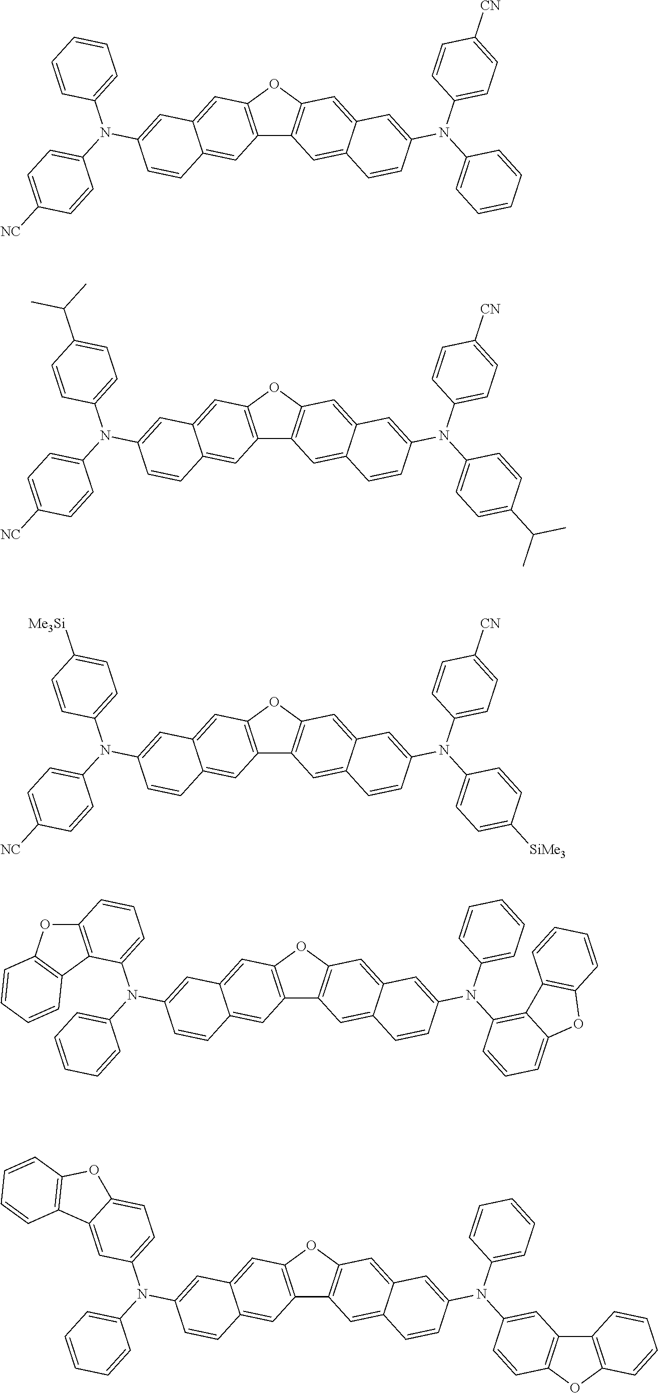 [SMILES: N#Cc1ccc(N(c2ccccc2)c2ccc3cc4c(cc3c2)oc2cc3cc(N(c5ccccc5)c5ccc(C#N)cc5)ccc3cc24)cc1 ; CC(C)c1ccc(N(c2ccc(C#N)cc2)c2ccc3cc4c(cc3c2)oc2cc3cc(N(c5ccc(C#N)cc5)c5ccc(C(C)C)cc5)ccc3cc24)cc1 ; C[Si](C)(C)c1ccc(N(c2ccc(C#N)cc2)c2ccc3cc4c(cc3c2)oc2cc3cc(N(c5ccc(C#N)cc5)c5ccc([Si](C)(C)C)cc5)ccc3cc24)cc1 ; c1ccc(N(c2ccc3cc4c(cc3c2)oc2cc3cc(N(c5ccccc5)c5cccc6oc7ccccc7c56)ccc3cc24)c2cccc3oc4ccccc4c23)cc1 ; c1ccc(N(c2ccc3cc4c(cc3c2)oc2cc3cc(N(c5ccccc5)c5ccc6oc7ccccc7c6c5)ccc3cc24)c2ccc3oc4ccccc4c3c2)cc1]

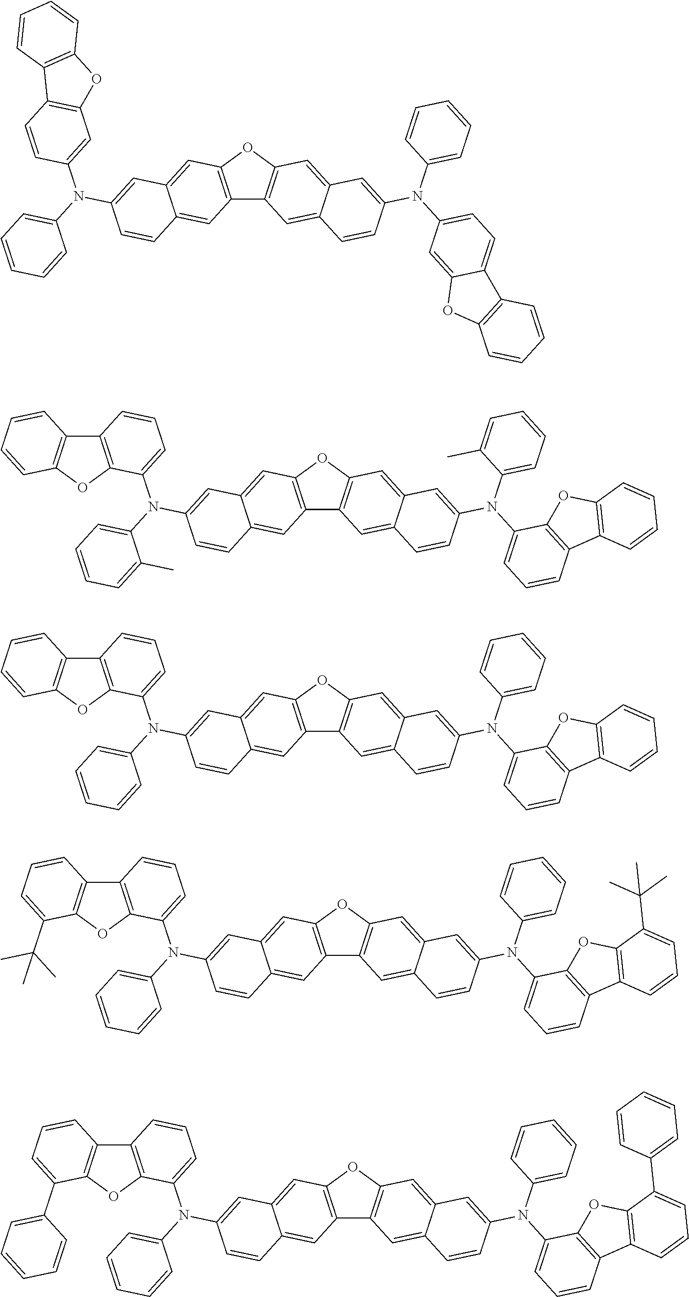

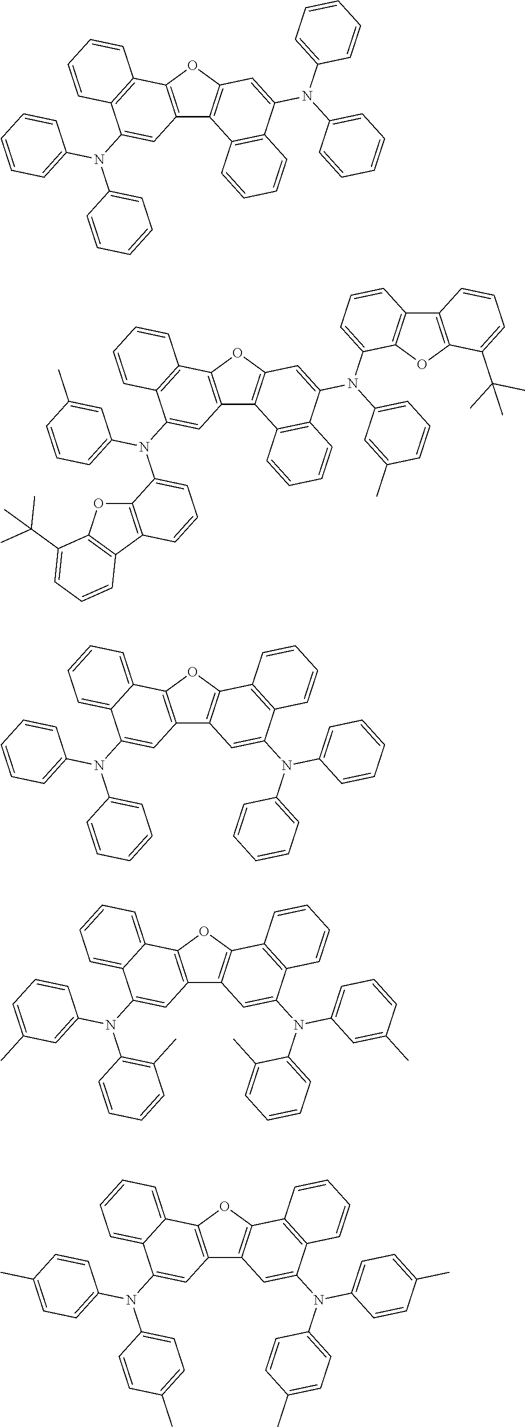

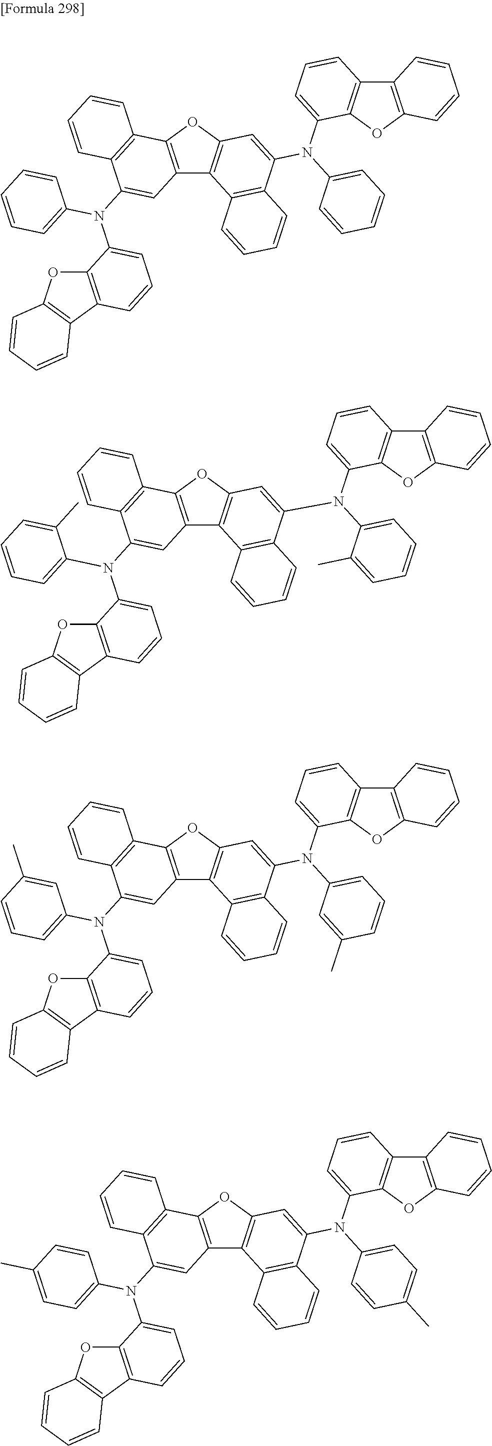 [SMILES: c1ccc(N(c2cc3c(oc4cc(N(c5ccccc5)c5cccc6c5oc5ccccc56)c5ccccc5c43)c3ccccc23)c2cccc3c2oc2ccccc23)cc1 ; Cc1ccccc1N(c1cc2c(oc3cc(N(c4ccccc4C)c4cccc5c4oc4ccccc45)c4ccccc4c32)c2ccccc12)c1cccc2c1oc1ccccc12 ; Cc1cccc(N(c2cc3c(oc4cc(N(c5cccc(C)c5)c5cccc6c5oc5ccccc56)c5ccccc5c43)c3ccccc23)c2cccc3c2oc2ccccc23)c1 ; Cc1ccc(N(c2cc3c(oc4cc(N(c5ccc(C)cc5)c5cccc6c5oc5ccccc56)c5ccccc5c43)c3ccccc23)c2cccc3c2oc2ccccc23)cc1]

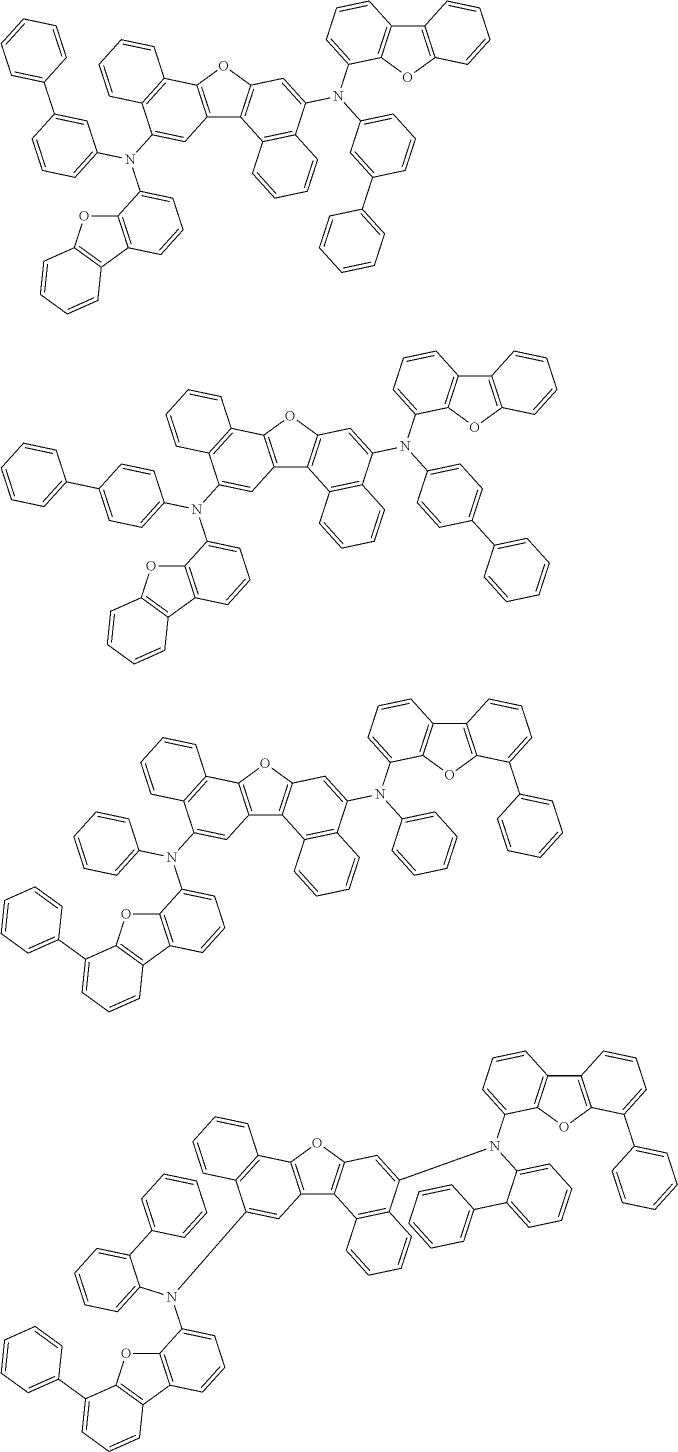

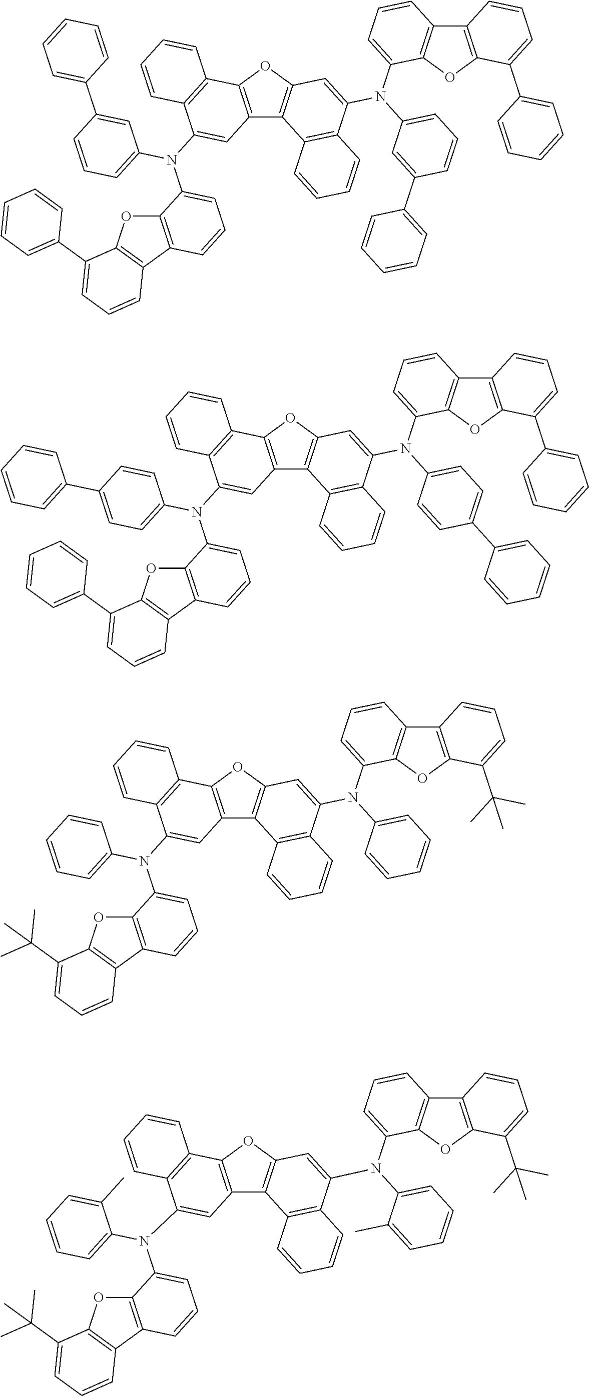

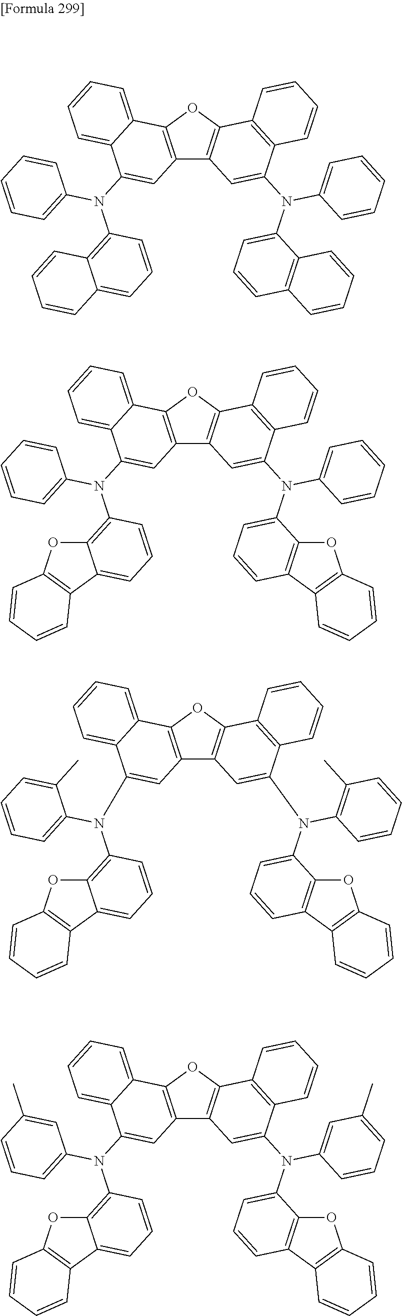 [SMILES: c1ccc(N(c2cccc3ccccc23)c2cc3c4cc(N(c5ccccc5)c5cccc6ccccc56)c5ccccc5c4oc3c3ccccc23)cc1 ; c1ccc(N(c2cc3c4cc(N(c5ccccc5)c5cccc6c5oc5ccccc56)c5ccccc5c4oc3c3ccccc23)c2cccc3c2oc2ccccc23)cc1 ; Cc1ccccc1N(c1cc2c3cc(N(c4ccccc4C)c4cccc5c4oc4ccccc45)c4ccccc4c3oc2c2ccccc12)c1cccc2c1oc1ccccc12 ; Cc1cccc(N(c2cc3c4cc(N(c5cccc(C)c5)c5cccc6c5oc5ccccc56)c5ccccc5c4oc3c3ccccc23)c2cccc3c2oc2ccccc23)c1]

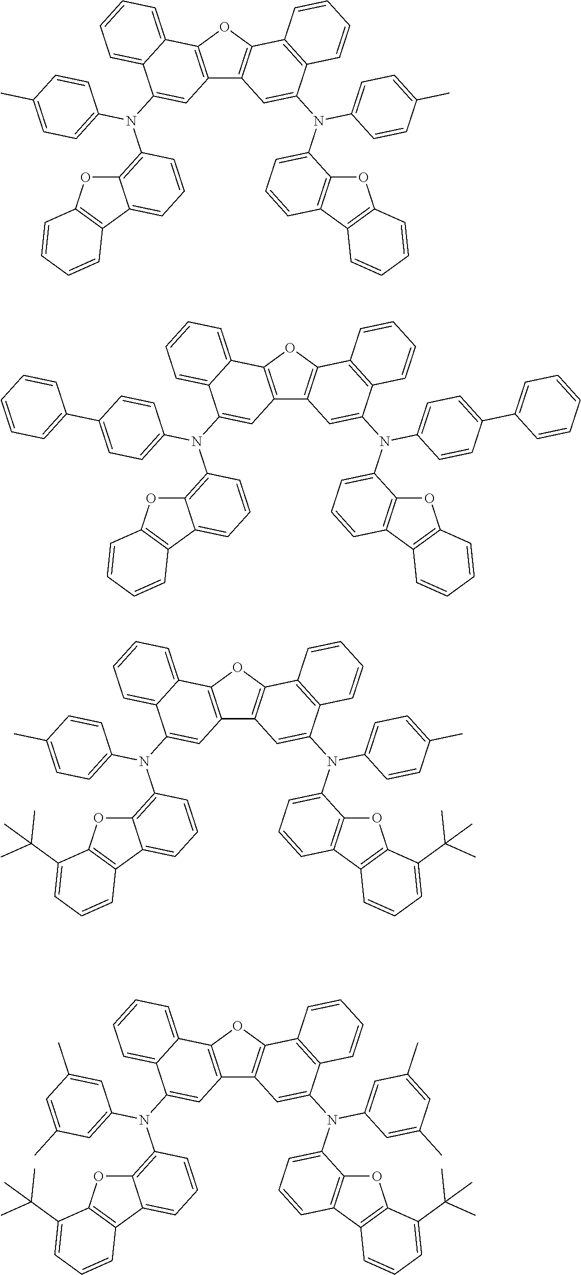

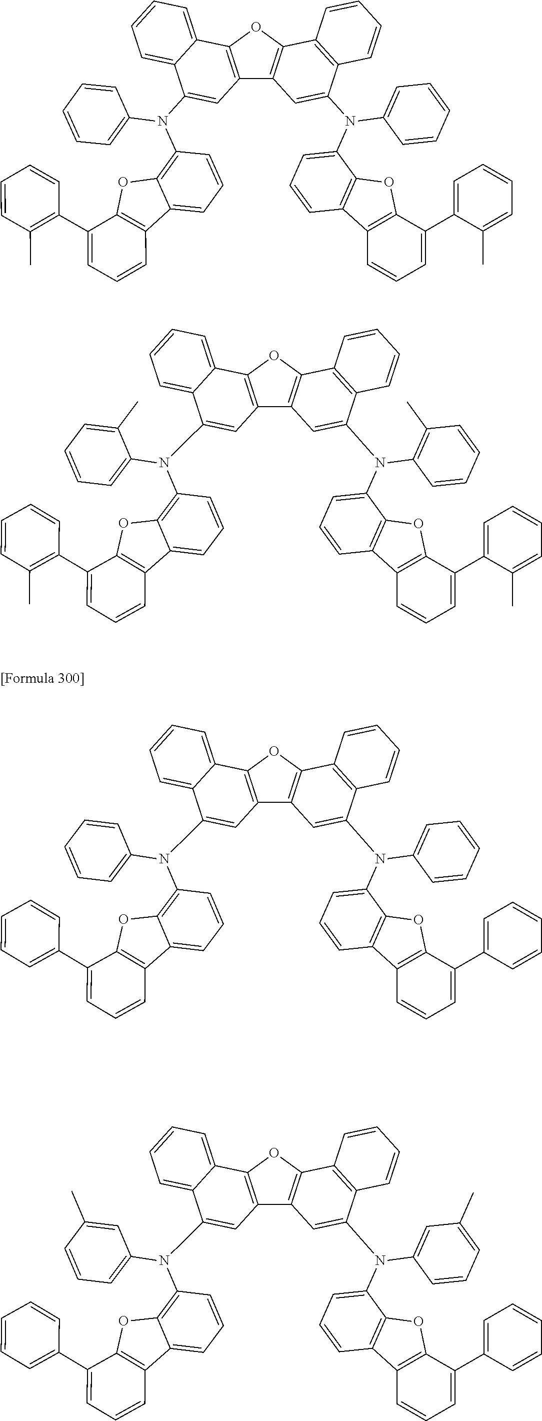 [SMILES: Cc1ccccc1-c1cccc2c1oc1c(N(c3ccccc3)c3cc4c5cc(N(c6ccccc6)c6cccc7c6oc6c(-c8ccccc8C)cccc67)c6ccccc6c5oc4c4ccccc34)cccc12 ; Cc1ccccc1-c1cccc2c1oc1c(N(c3ccccc3C)c3cc4c5cc(N(c6ccccc6C)c6cccc7c6oc6c(-c8ccccc8C)cccc67)c6ccccc6c5oc4c4ccccc34)cccc12 ; c1ccc(-c2cccc3c2oc2c(N(c4ccccc4)c4cc5c6cc(N(c7ccccc7)c7cccc8c7oc7c(-c9ccccc9)cccc78)c7ccccc7c6oc5c5ccccc45)cccc23)cc1 ; Cc1cccc(N(c2cc3c4cc(N(c5cccc(C)c5)c5cccc6c5oc5c(-c7ccccc7)cccc56)c5ccccc5c4oc3c3ccccc23)c2cccc3c2oc2c(-c4ccccc4)cccc23)c1]

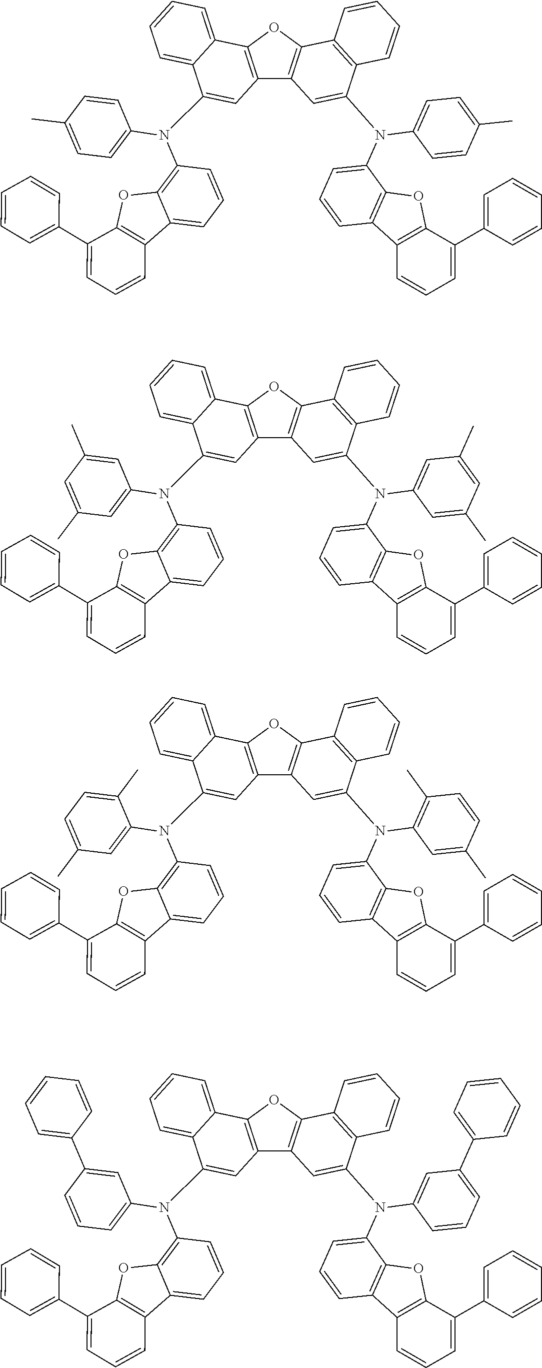

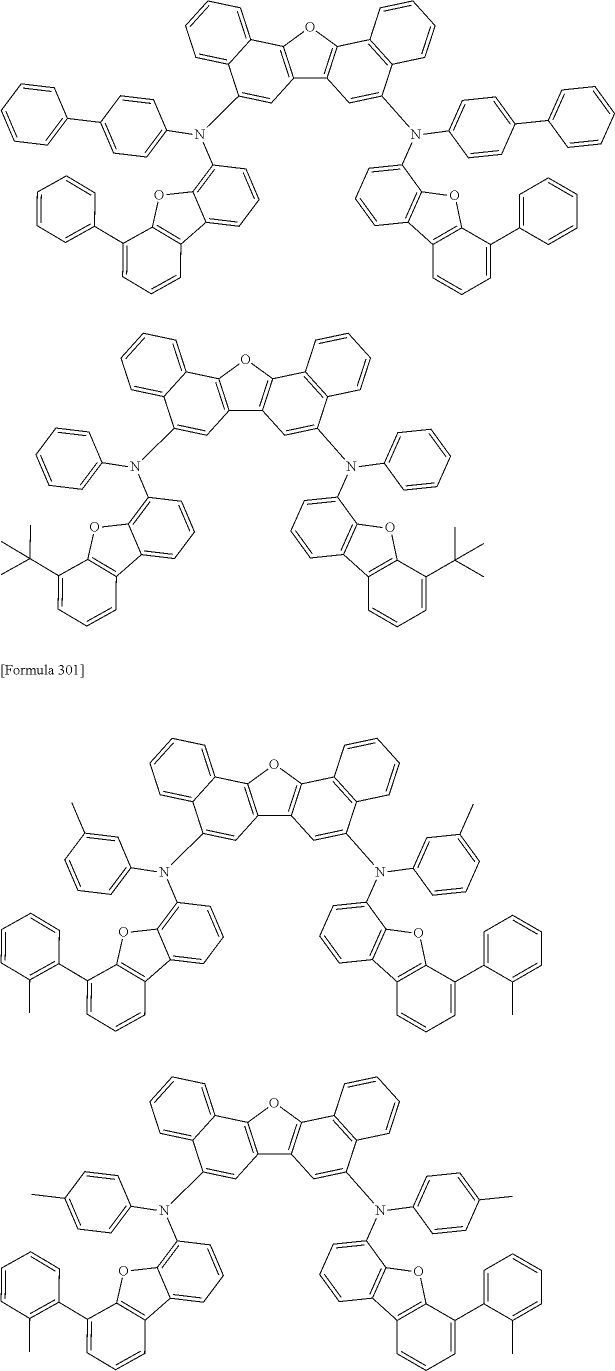

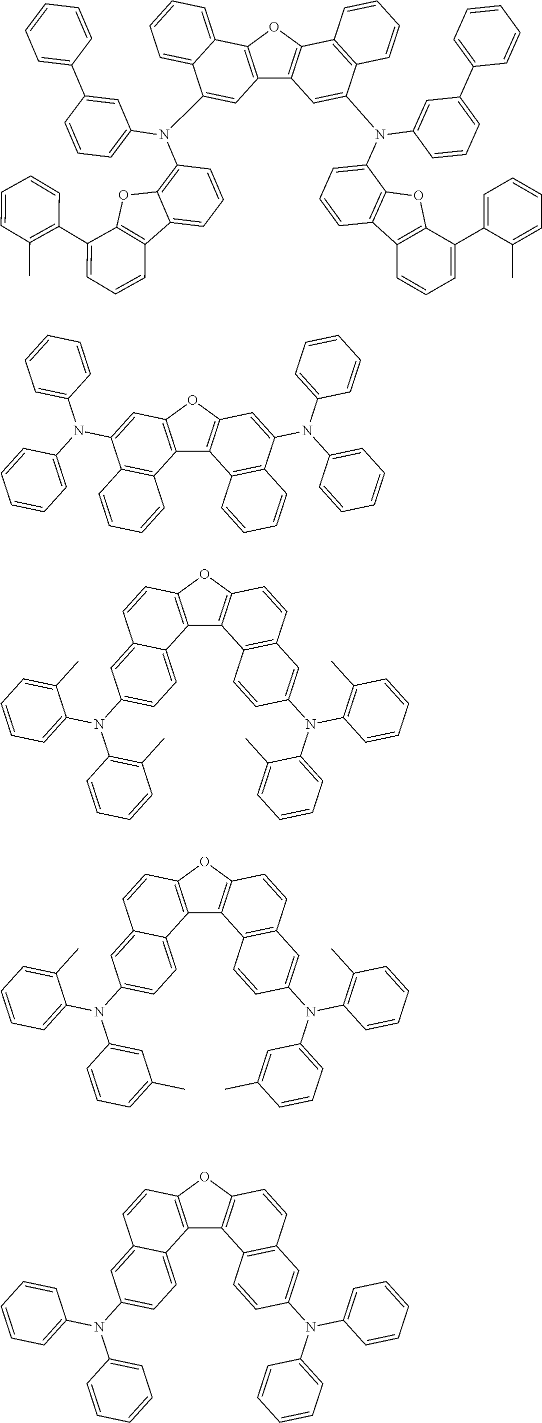 [SMILES: Cc1ccccc1-c1cccc2c1oc1c(N(c3cccc(-c4ccccc4)c3)c3cc4c5cc(N(c6cccc(-c7ccccc7)c6)c6cccc7c6oc6c(-c8ccccc8C)cccc67)c6ccccc6c5oc4c4ccccc34)cccc12 ; c1ccc(N(c2ccccc2)c2cc3oc4cc(N(c5ccccc5)c5ccccc5)c5ccccc5c4c3c3ccccc23)cc1 ; Cc1ccccc1N(c1ccc2c(ccc3oc4ccc5cc(N(c6ccccc6C)c6ccccc6C)ccc5c4c32)c1)c1ccccc1C ; Cc1cccc(N(c2ccc3c(ccc4oc5ccc6cc(N(c7cccc(C)c7)c7ccccc7C)ccc6c5c43)c2)c2ccccc2C)c1 ; c1ccc(N(c2ccccc2)c2ccc3c(ccc4oc5ccc6cc(N(c7ccccc7)c7ccccc7)ccc6c5c43)c2)cc1]

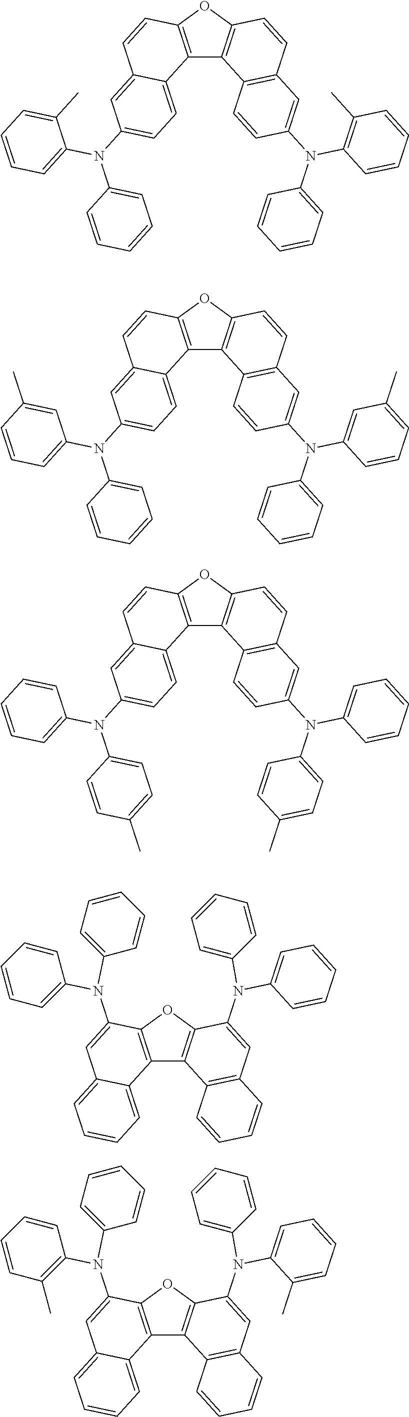

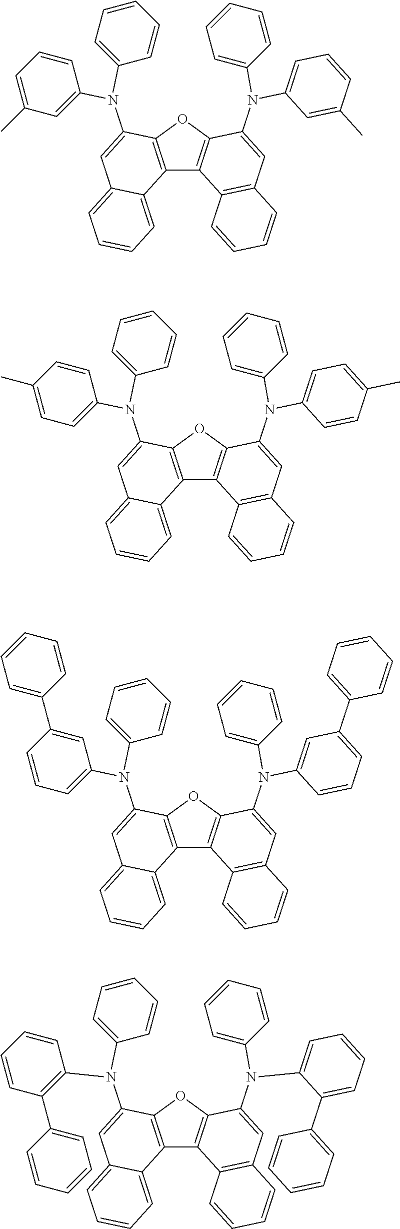

(Compound Represented by Formula (9))

A compound represented by the formula (9) is described below.

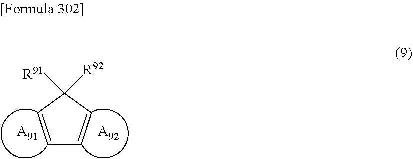

In the formula (9),
the A₉₁ ring and the A₉₂ ring are each independently a substituted or unsubstituted aromatic hydrocarbon ring having 6 to 50 ring carbon atoms, or a substituted or unsubstituted heterocycle having 5 to 50 ring atoms, and
at least one ring selected from the group consisting of the A₉₁ ring and the A₉₂ ring is bonded to * of the structure represented by the following formula (92).

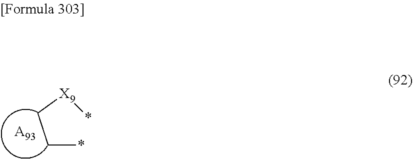

In the formula (92),
the A₉₃ ring is a substituted or unsubstituted aromatic hydrocarbon ring having 6 to 50 ring carbon atoms, or a substituted or unsubstituted heterocycle having 5 to 50 ring atoms,
X₉ represents NR₉₃, C(R₉₄)(R₉₅), Si(R₉₆)(R₉₇), Ge(R₉₈)(R₉₉), an oxygen atom, a sulfur atom, or a selenium atom,
R₉₁ and R₉₂ are mutually bonded to form a substituted or unsubstituted monocyclic ring, are mutually bonded to form a substituted or unsubstituted fused ring, or are not mutually bonded, and
R₉₁, R₉₂ that do not form the monocyclic ring and do not form the fused ring, and R₉₃ to R₉₉ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one ring selected from the group consisting of the A₉₁ ring and the A₉₂ ring is bonded to * of the structure represented by the formula (92). More specifically, in an exemplary embodiment, a ring carbon atom of the aromatic hydrocarbon ring or a ring atom of the heterocycle of the A₉₁ ring is bonded to * of the structure represented by the formula (92). Furthermore, in an exemplary embodiment, a ring carbon atom of the aromatic hydrocarbon ring or a ring atom of the heterocycle of the A₉₂ ring is bonded to * of the structure represented by the formula (92).

In an exemplary embodiment, one or both of the A₉₁ ring and the A₉₂ ring are bonded to a group represented by the following formula (93).

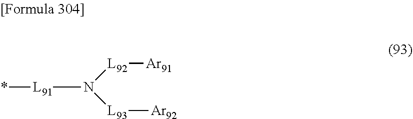

In the formula (93),
Ar₉₁ and Ar₉₂ each independently represent a substituted or unsubstituted aryl group having 6 to 50 ring carbon atoms, or a substituted or unsubstituted heterocyclic group having 5 to 50 ring atoms,
L₉₁ to L₉₃ each independently represent a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms, and
* in the formula (93) represents a bonding position to one of A₉₁ ring and A₉₂ ring.

In an exemplary embodiment, in addition to the A₉₁ ring, a ring carbon atom of the aromatic hydrocarbon ring or a ring atom of the heterocycle of the A₉₂ ring is bonded to * of the structure represented by the formula (92). In this case, the structure represented by the formula (92) may be mutually the same or different.

In an exemplary embodiment, R₉₁ and R₉₂ each independently represent a substituted or unsubstituted aryl group having 6 to 50 ring carbon atoms.

In an exemplary embodiment, R₉₁ and R₉₂ are mutually bonded to form a fluorene structure.

In an exemplary embodiment, the A₉₁ ring and the A₉₂ ring each independently represent a substituted or unsubstituted aromatic hydrocarbon ring having 6 to 50 ring carbon atoms, for example, a substituted or unsubstituted benzene ring.

In an exemplary embodiment, the A₉₃ ring is a substituted or unsubstituted aromatic hydrocarbon ring having 6 to 50 ring carbon atoms, for example, a substituted or unsubstituted benzene ring.

In an exemplary embodiment, X₉ represents an oxygen atom or a sulfur atom.

Specific examples of a compound represented by the formula (9) include the following compounds.

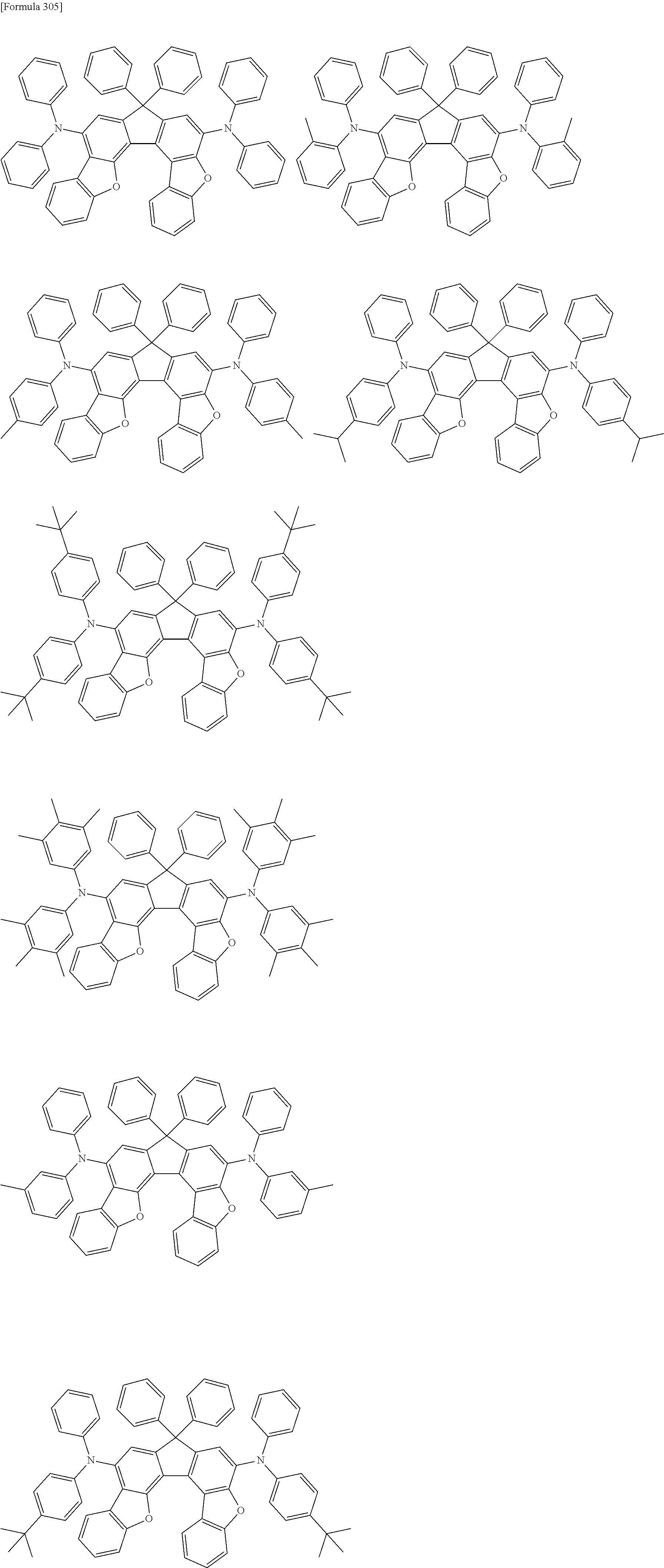

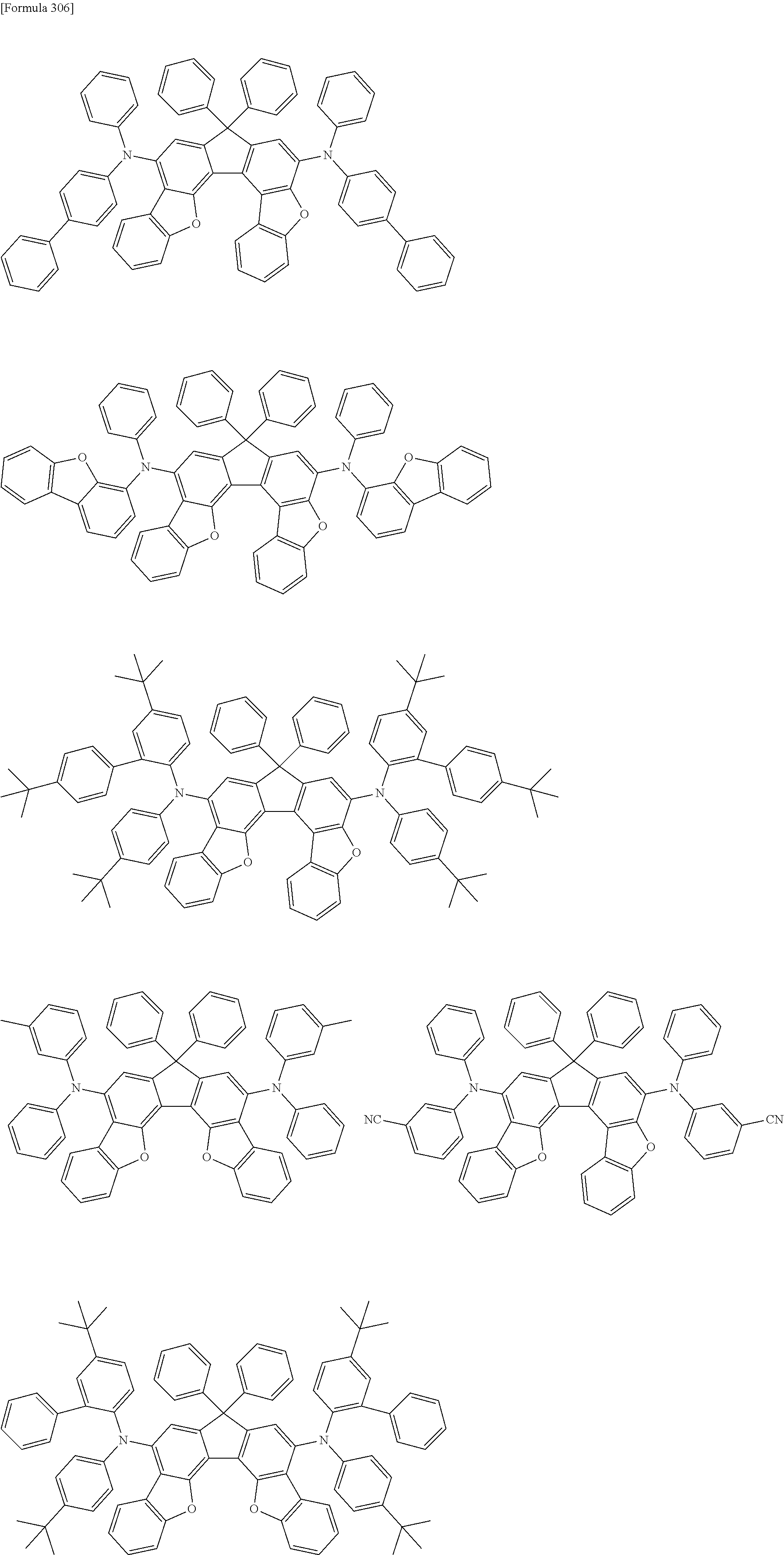

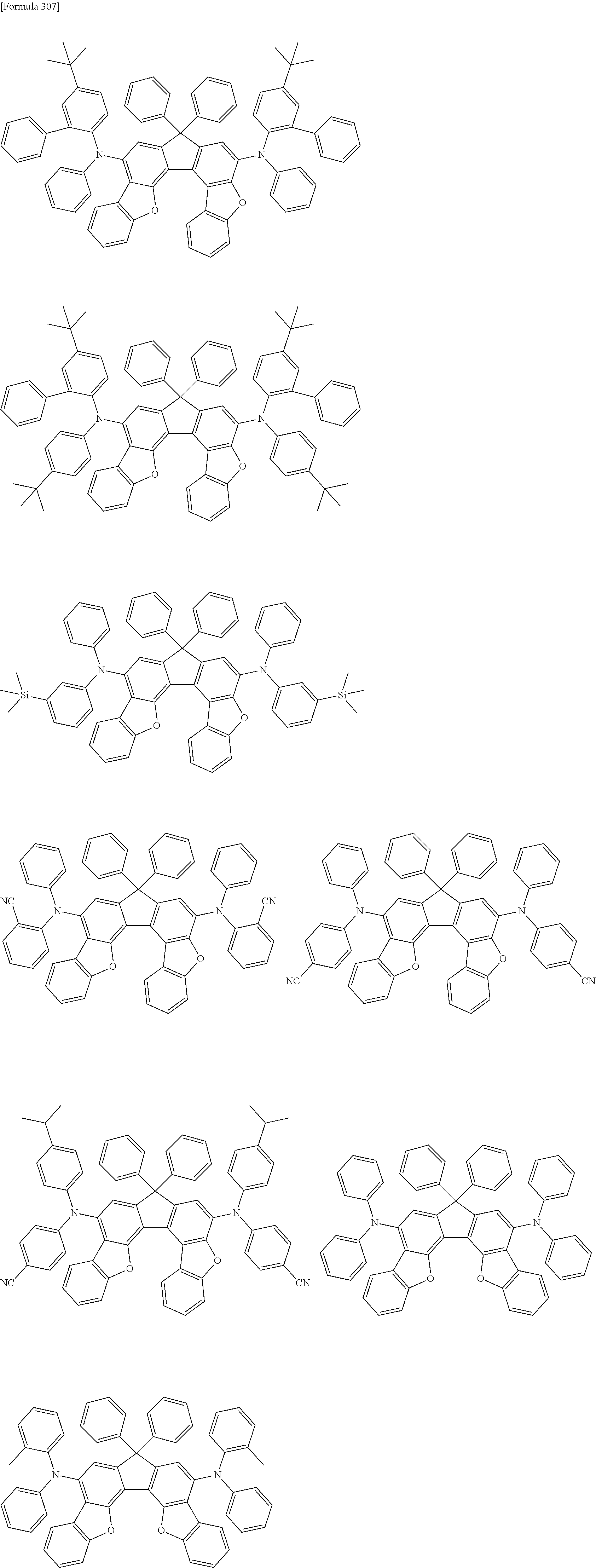

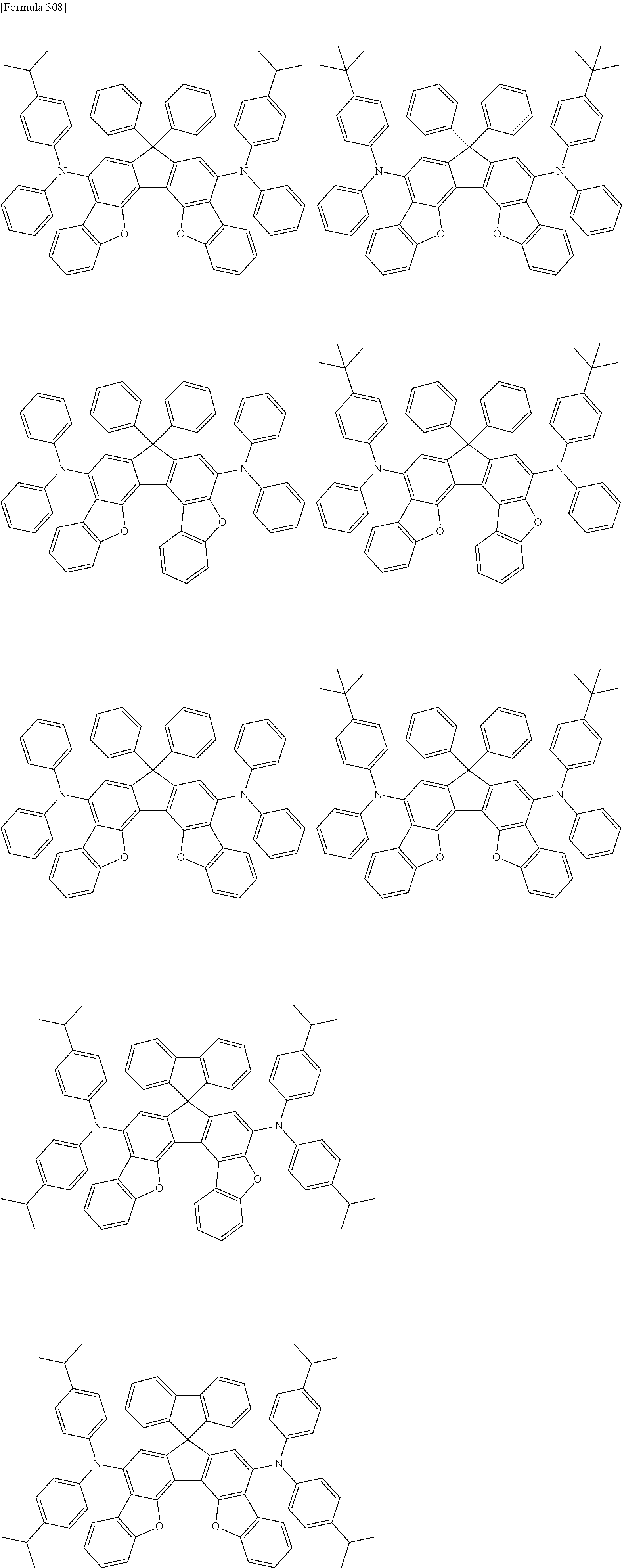

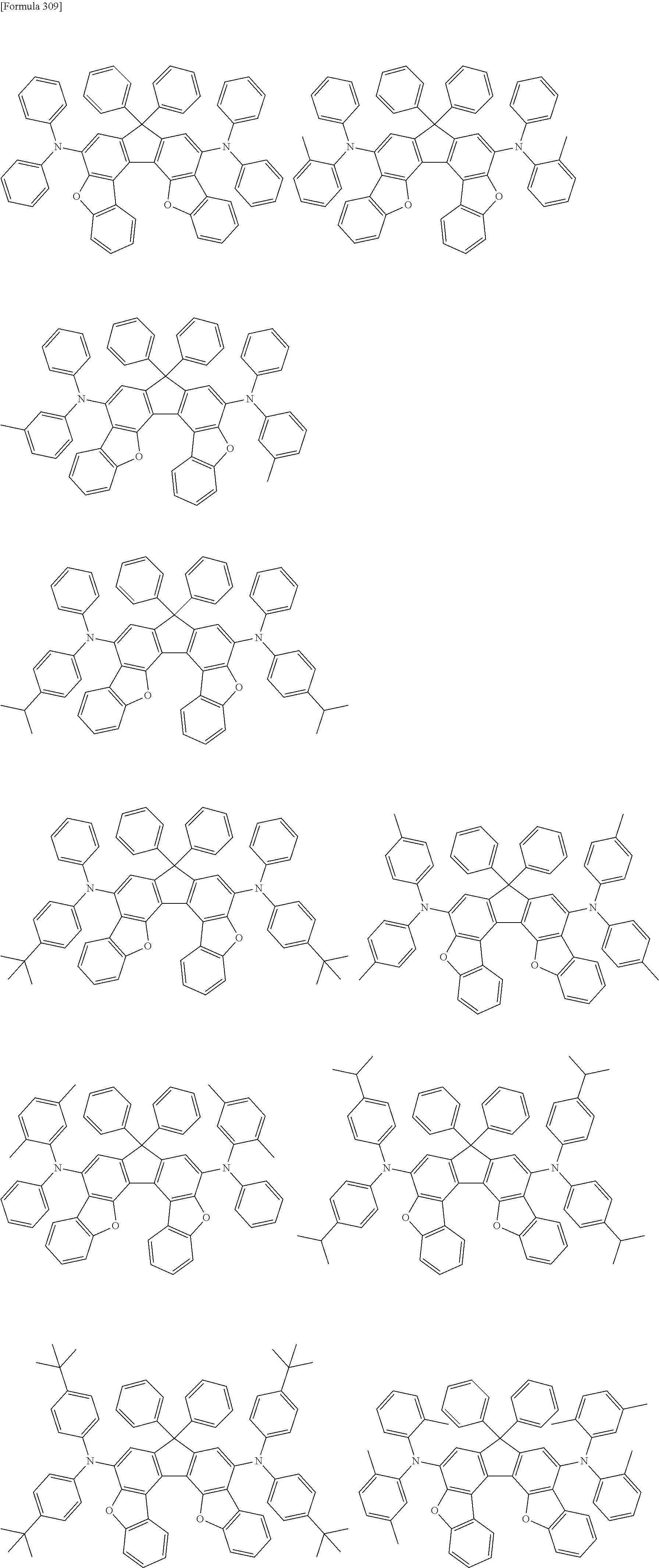

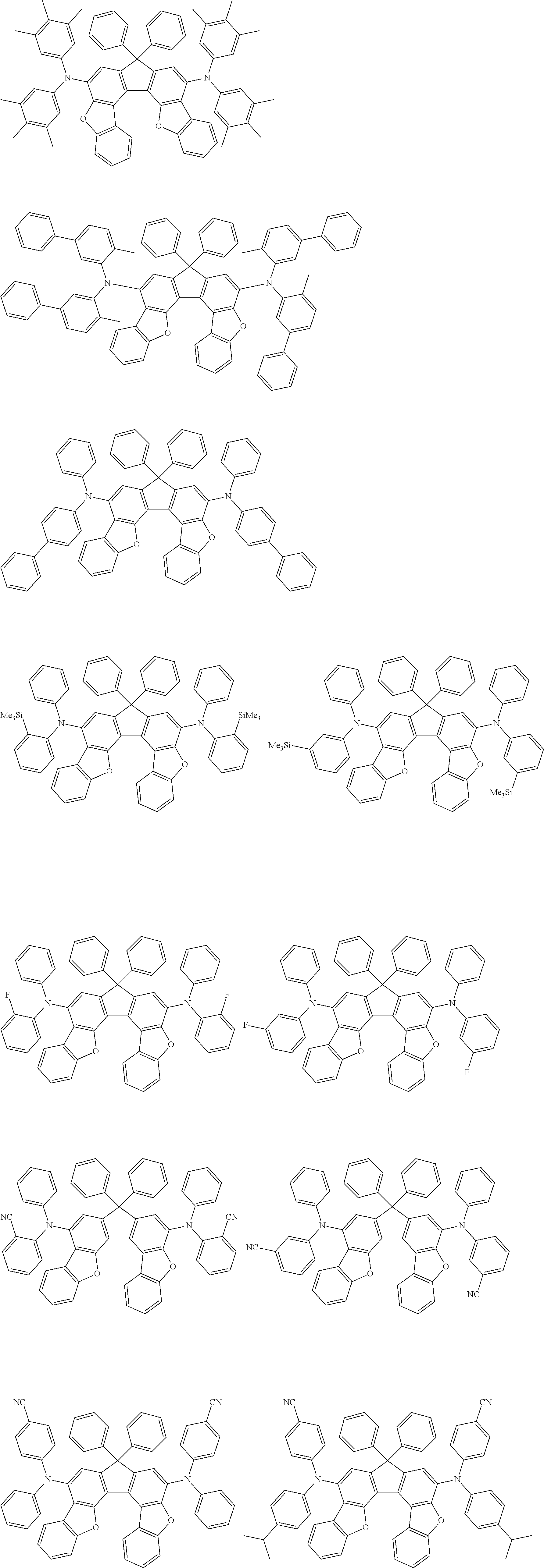

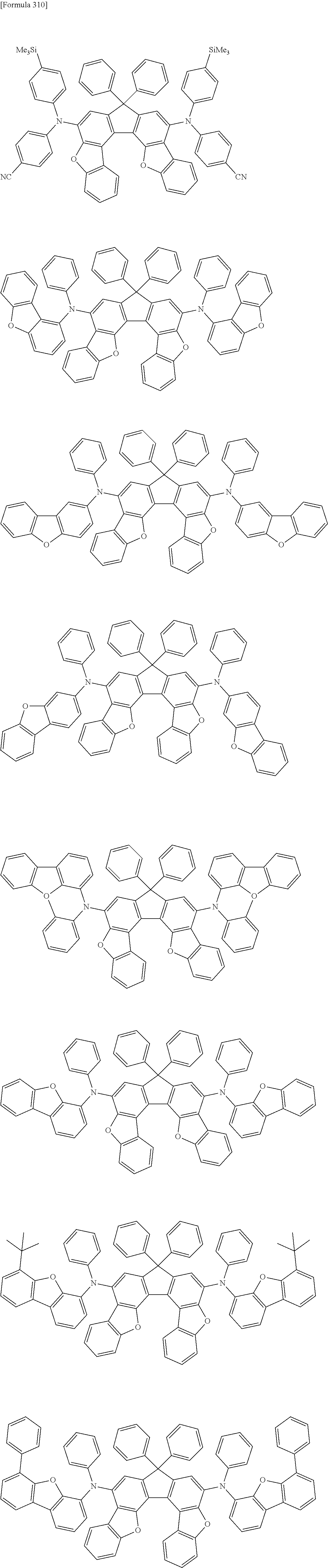

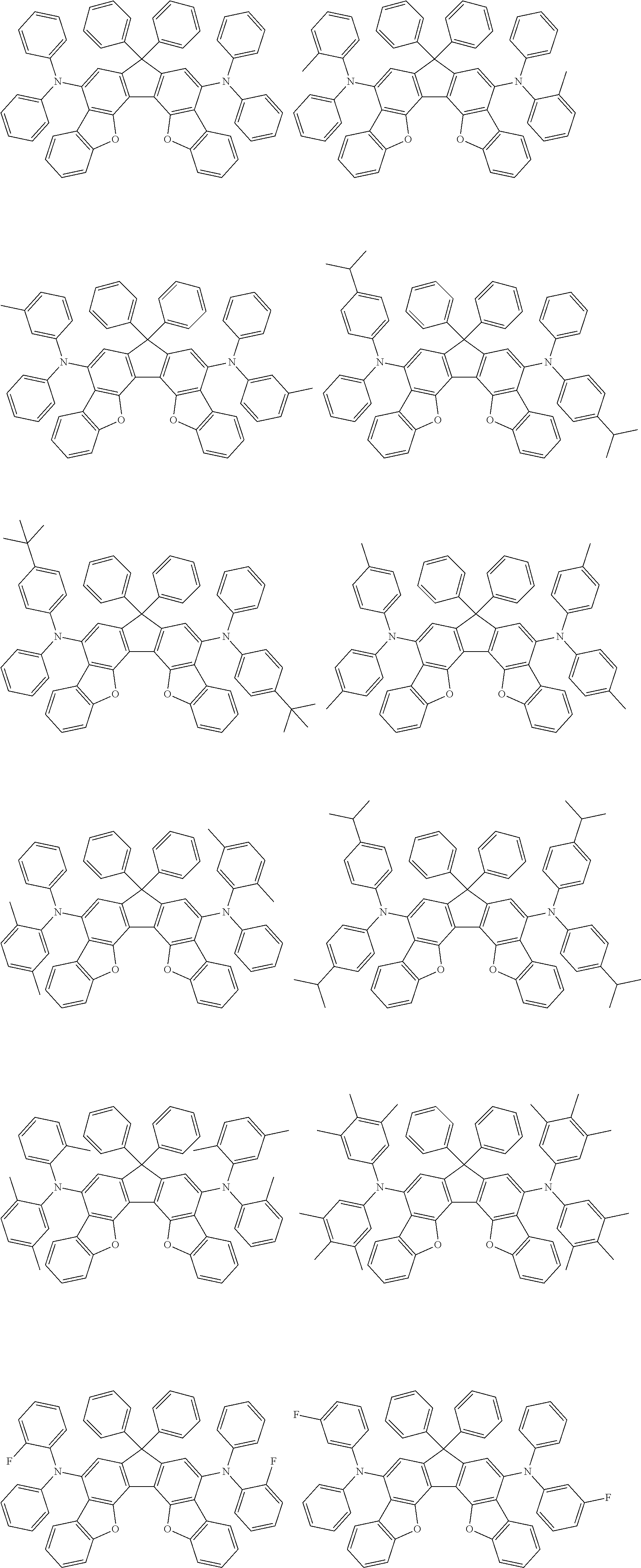

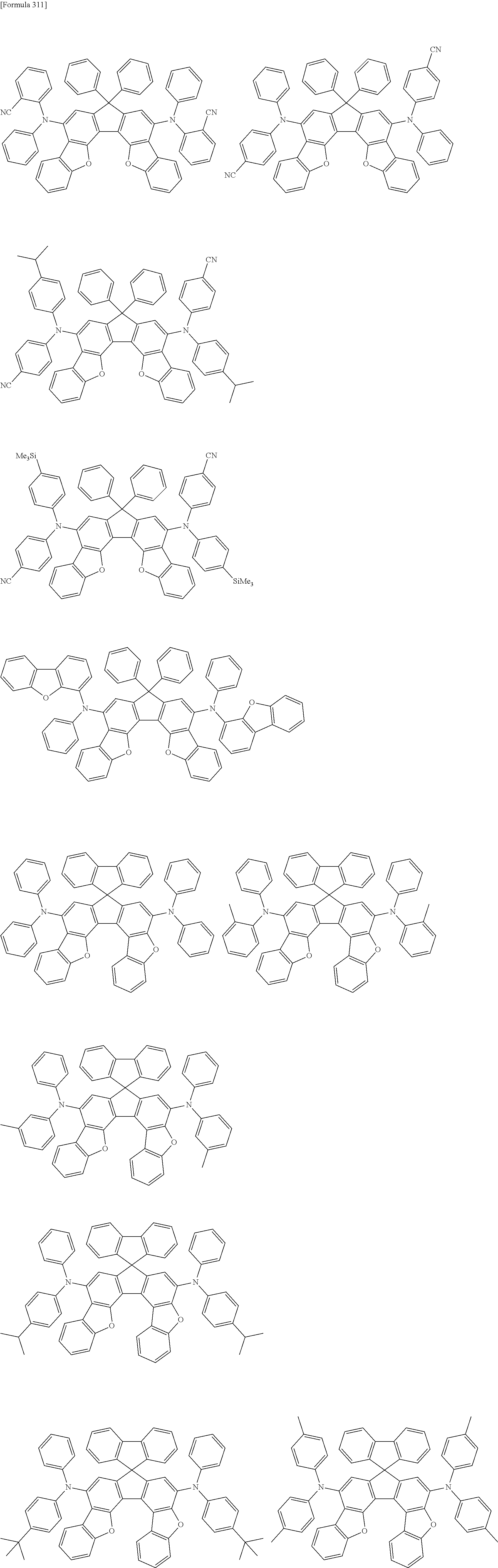

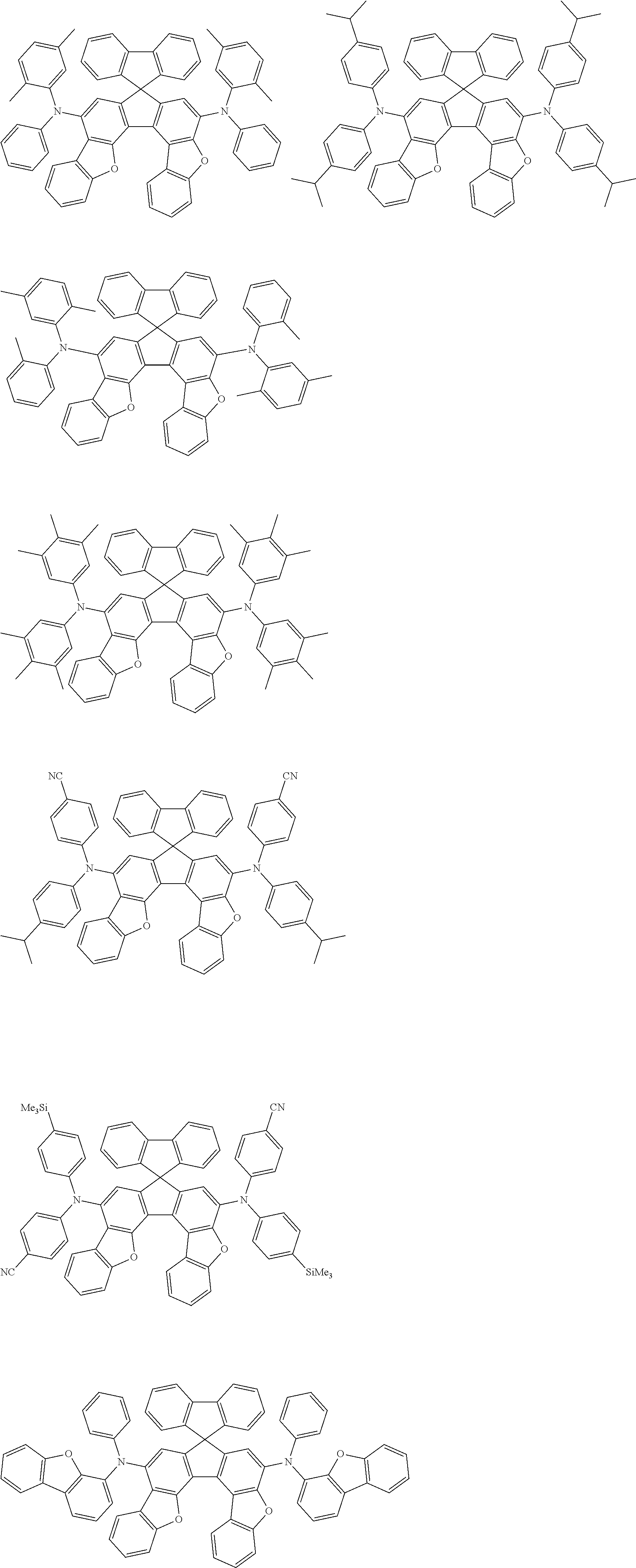

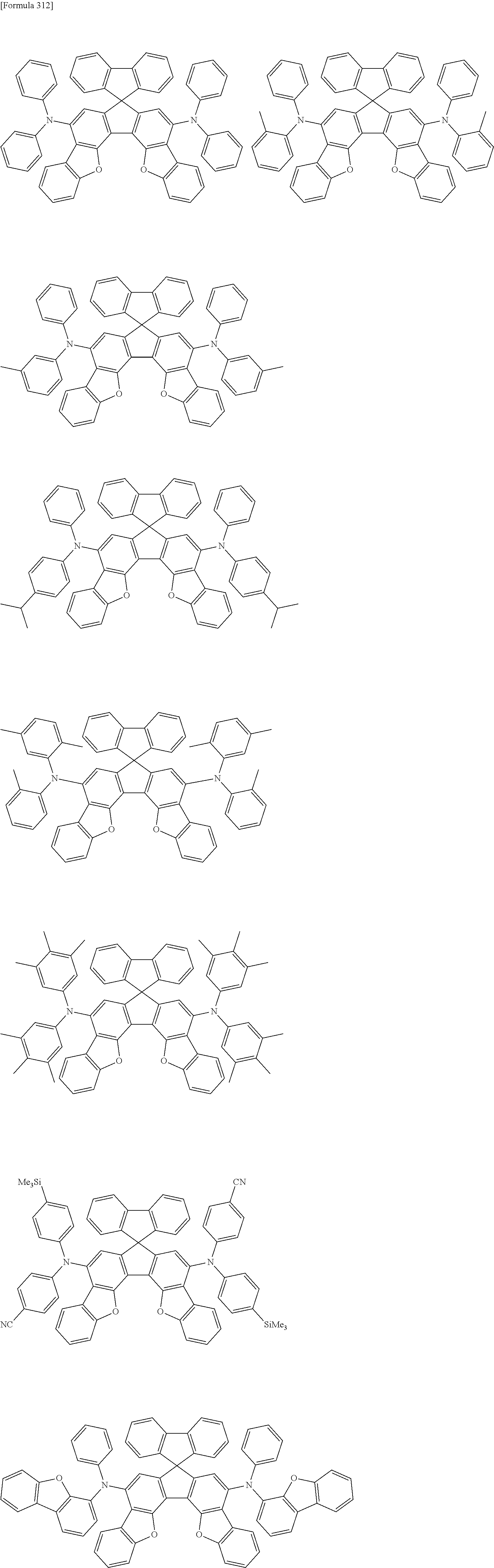

Compound Represented by Formula (10)

A compound represented by the formula (10) is described below.

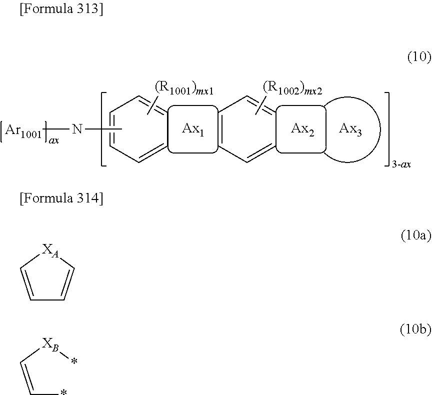

In the formula (10),
the Ax₁ ring is a ring represented by the formula (10a) and fused at any position of an adjacent ring,
the Ax₂ ring is a ring represented by the formula (10b) and fused at any position of an adjacent ring,
two * in the formula (10b) are bonded to the Ax₃ ring at any positions,
X_{A} and X_{B} each independently represent C(R₁₀₀₃)(R₁₀₀₄), Si(R₁₀₀₅)(R₁₀₀₆), an oxygen atom, or a sulfur atom,
the Ax₃ ring is a substituted or unsubstituted aromatic hydrocarbon ring having 6 to 50 ring carbon atoms, or a substituted or unsubstituted heterocycle having 5 to 50 ring atoms,
Ar₁₀₀₁ represents a substituted or unsubstituted aryl group having 6 to 50 ring carbon atoms, or a substituted or unsubstituted heterocyclic group having 5 to 50 ring atoms,
R₁₀₀₁ to R₁₀₀₆ each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms,
mx1 is 3, mx2 is 2,
a plurality of R₁₀₀₁ are mutually the same or different,
a plurality of R₁₀₀₂ are mutually the same or different,
ax is 0, 1, or 2,
when ax is 0 or 1, the structures in the square brackets represented by “3-ax” are mutually the same or different, and
when ax is 2, a plurality of Ar₁₀₀₁ are mutually the same or different.

In an exemplary embodiment, Ar₁₀₀₁ represents a substituted or unsubstituted aryl group having 6 to 50 ring carbon atoms.

In an exemplary embodiment, the Ax₃ ring is a substituted or unsubstituted aromatic hydrocarbon ring having 6 to 50 ring carbon atoms, for example, a substituted or unsubstituted benzene ring, a substituted or unsubstituted naphthalene ring, or a substituted or unsubstituted anthracene ring.

In an exemplary embodiment, R₁₀₀₃ and R₁₀₀₄ each independently represent a substituted or unsubstituted alkyl group having 1 to 50 carbon atoms.

In an exemplary embodiment, ax is 1.

Specific examples of a compound represented by the formula (10) include the following compounds.

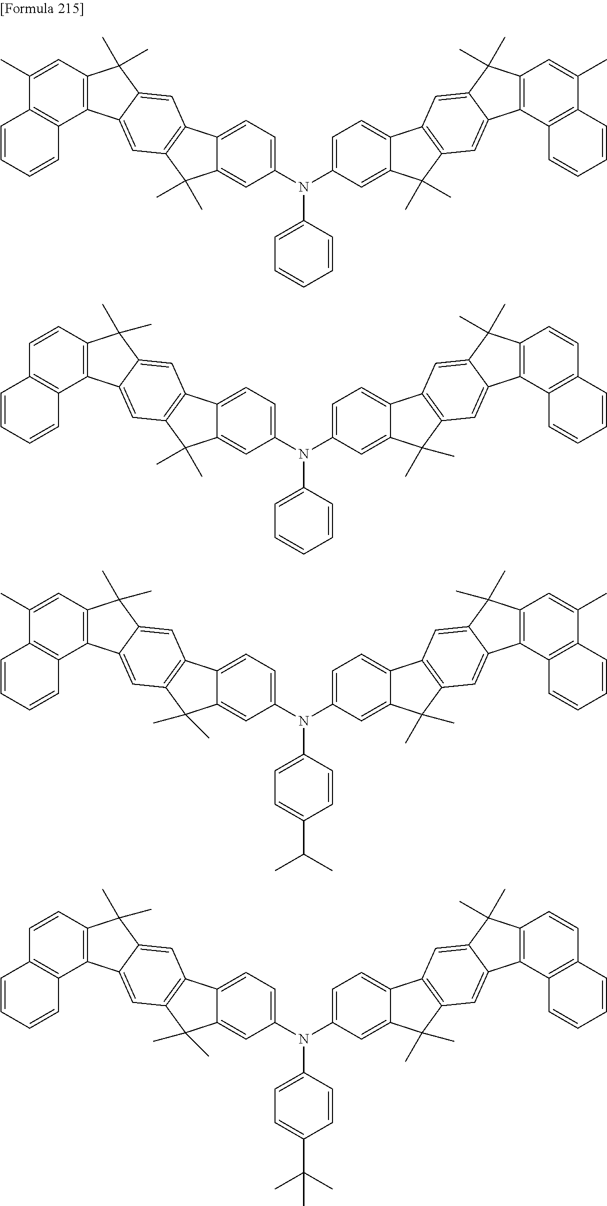

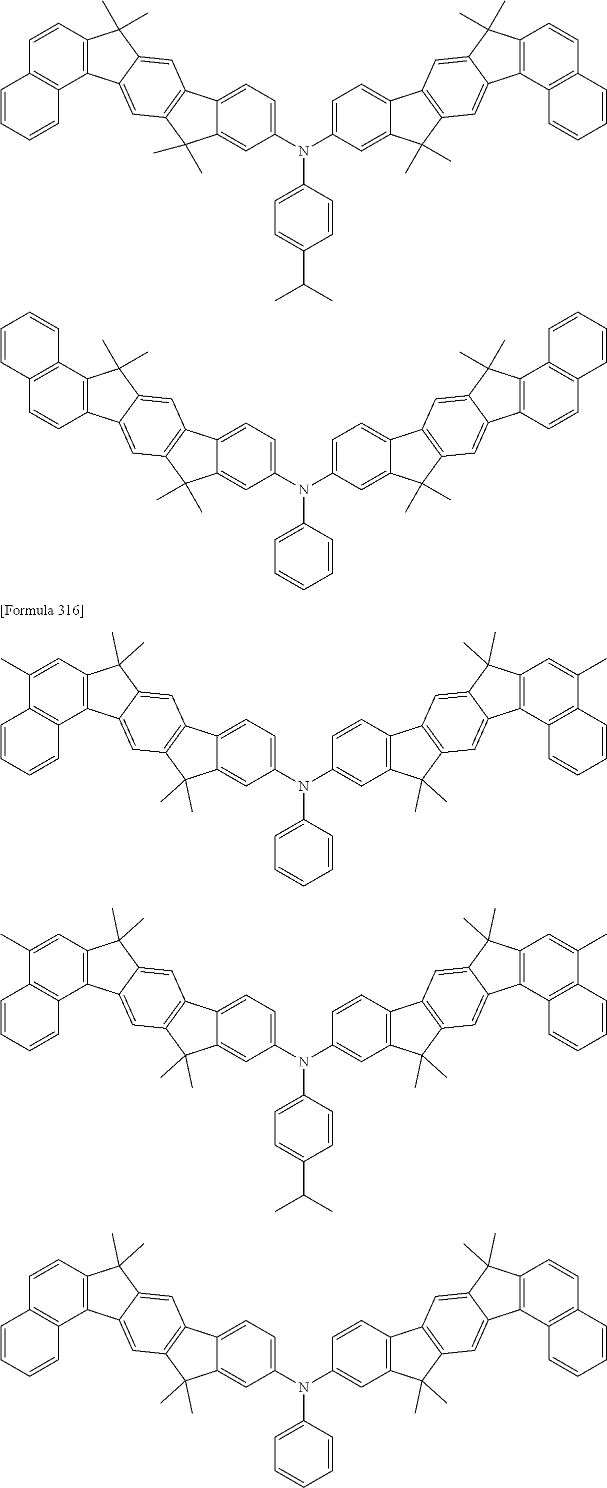

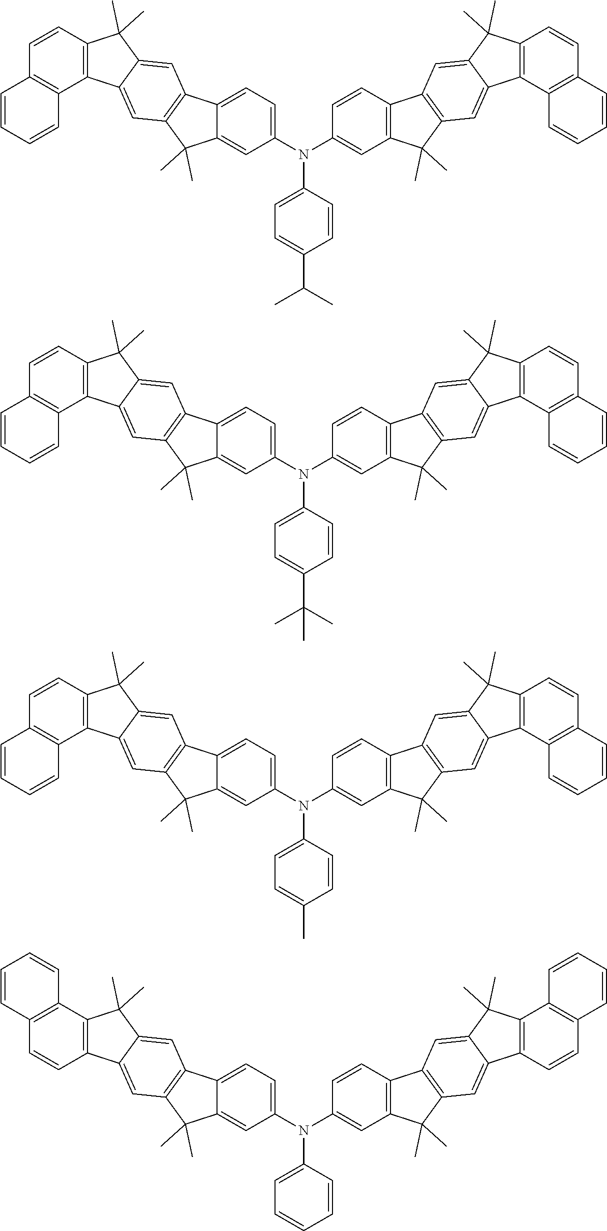

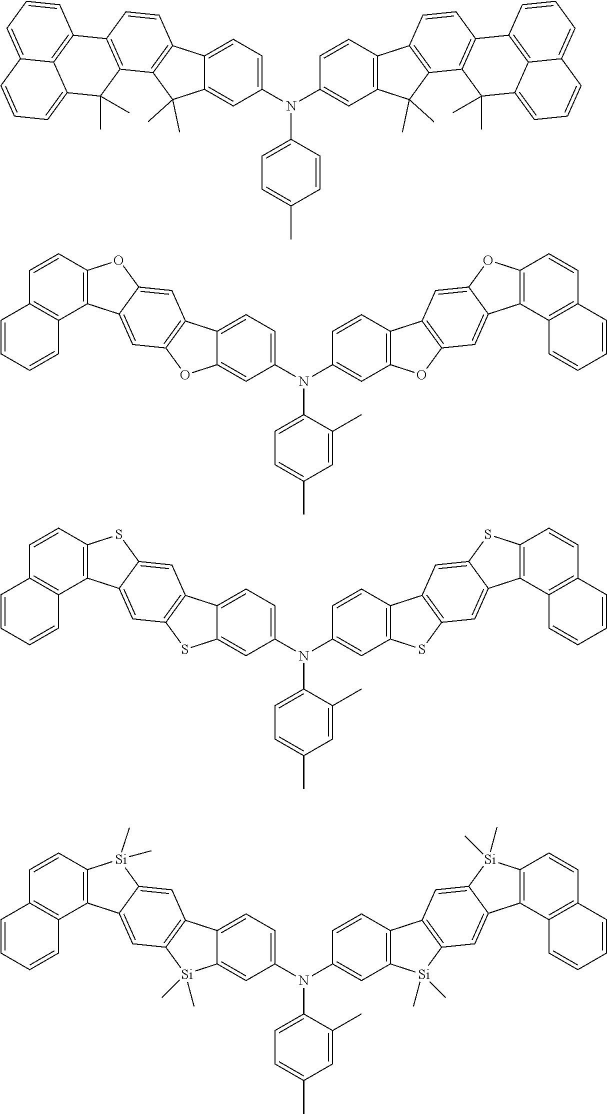 [SMILES: Cc1ccc(N(c2ccc3c(c2)C(C)(C)c2c-3ccc3c2C(C)(C)c2cccc4cccc-3c24)c2ccc3c(c2)C(C)(C)c2c-3ccc3c2C(C)(C)c2cccc4cccc-3c24)cc1 ; Cc1ccc(N(c2ccc3c(c2)oc2cc4c(cc23)oc2ccc3ccccc3c24)c2ccc3c(c2)oc2cc4c(cc23)oc2ccc3ccccc3c24)c(C)c1 ; Cc1ccc(N(c2ccc3c(c2)sc2cc4c(cc23)sc2ccc3ccccc3c24)c2ccc3c(c2)sc2cc4c(cc23)sc2ccc3ccccc3c24)c(C)c1 ; Cc1ccc(N(c2ccc3c(c2)[Si](C)(C)c2cc4c(cc2-3)[Si](C)(C)c2ccc3ccccc3c2-4)c2ccc3c(c2)[Si](C)(C)c2cc4c(cc2-3)[Si](C)(C)c2ccc3ccccc3c2-4)c(C)c1]

In an exemplary embodiment, the emitting layer contains the compound represented by the formula (120), and as a fluorescent compound (the compound M1) at least one compound selected from the group consisting of compounds represented by the formula (100), compounds represented by the formula (4), compounds represented by the formula (5), compounds represented by the formula (7), compounds represented by the formula (8), compounds represented by the formula (9), and compounds represented by the following formula (63a).

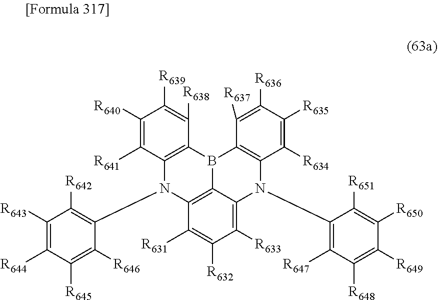

In the formula (63a),
R₆₃₁ is bonded to R₆₄₆ to form a substituted or unsubstituted heterocycle, or does not form a substituted or unsubstituted heterocycle.
R₆₃₃ is bonded to R₆₄₇ to form a substituted or unsubstituted heterocycle, or does not form a substituted or unsubstituted heterocycle.
R₆₃₄ is bonded to R₆₅₁ to form a substituted or unsubstituted heterocycle, or does not form a substituted or unsubstituted heterocycle.
R₆₄₁ is bonded to R₆₄₂ to form a substituted or unsubstituted heterocycle, or does not form a substituted or unsubstituted heterocycle.

At least one combination of adjacent two or more of R₆₃₁ to R₆₅₁ are mutually bonded to form a substituted or unsubstituted monocyclic ring, are mutually bonded to form a substituted or unsubstituted fused ring, or are not mutually bonded,
R₆₃₁ to R₆₅₁ that do not form the substituted or unsubstituted heterocycle, do not form the monocyclic ring, and do not form the fused ring each independently represent a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms,
at least one of R₆₃₁ to R₆₅₁ that do not form the substituted or unsubstituted heterocycle, do not form the monocyclic ring, and do not form the fused ring represents a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, a compound represented by the formula (4) is a compound represented by the formula (41-3), the formula (41-4), or the formula (41-5), and the A1 ring in the formula (41-5) is a substituted or unsubstituted fused aromatic hydrocarbon ring having 10 to 50 ring carbon atoms or a substituted or unsubstituted fused heterocycle having 8 to 50 ring atoms.

In an exemplary embodiment, a substituted or unsubstituted fused aromatic hydrocarbon ring having 10 to 50 ring carbon atoms in the formula (41-3), the formula (41-4), and the formula (41-5) is a substituted or unsubstituted naphthalene ring, a substituted or unsubstituted anthracene ring, or a substituted or unsubstituted fluorene ring, and
a substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, a substituted or unsubstituted fused aromatic hydrocarbon ring having 10 to 50 ring carbon atoms in the formula (41-3), the formula (41-4), or the formula (41-5) is a substituted or unsubstituted naphthalene ring, a substituted or unsubstituted fluorene ring, and
a substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, a compound represented by the formula (4) is selected from the group consisting of a compound represented by the following formula (461), a compound s represented by the following formula (462), a compound represented by the following formula (463), a compound represented by the following formula (464), a compound represented by the following formula (465), a compound represented by the following formula (466), and a compound represented by the following formula (467).

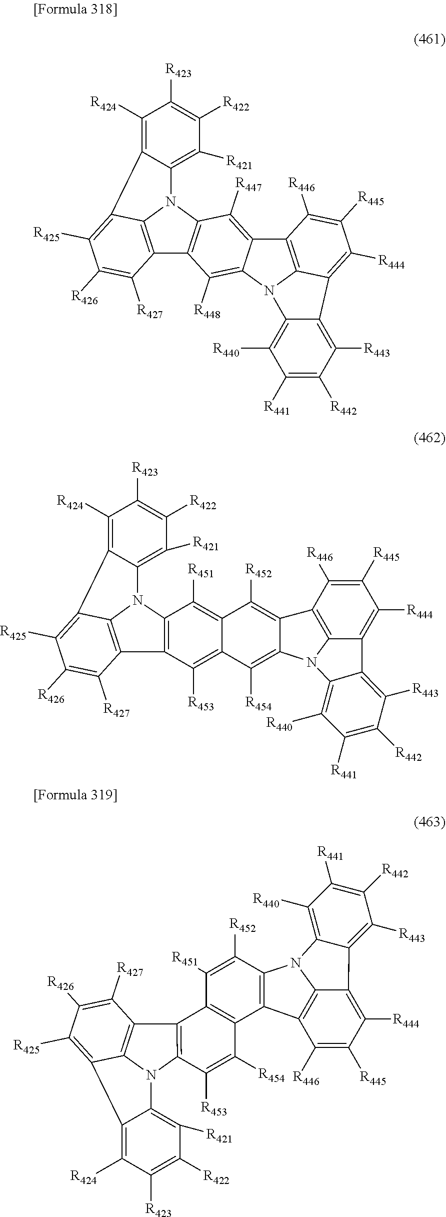

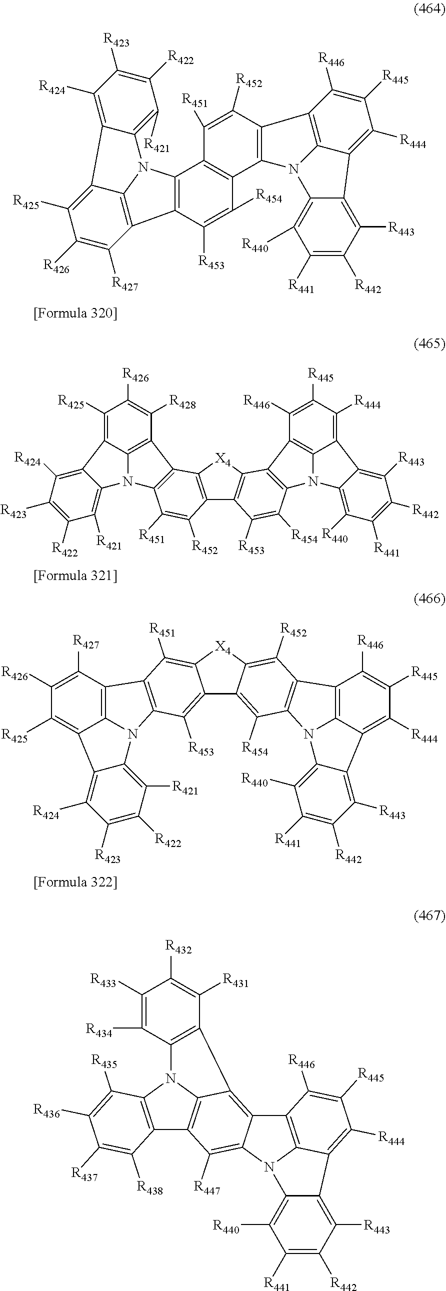

In the formula (461) to (467),
at least one combination of adjacent two or more of R₄₂₁ to R₄₂₇, R₄₃₁ to R₄₃₆, R₄₄₀ to R₄₄₈, and R₄₅₁ to R₄₅₄ are mutually bonded to form a substituted or unsubstituted monocyclic ring, are mutually bonded to form a substituted or unsubstituted fused ring, or are not mutually bonded,
R₄₃₇ and R₄₃₈, and R₄₂₁ to R₄₂₇, R₄₃₁ to R₄₃₆, R₄₄₀ to R₄₄₈ and R₄₅₁ to R₄₅₄ that do not form the monocyclic ring and do not form the fused ring each independently represent a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms,
X₄ represents an oxygen atom, NR₈₀₁, or C(R₈₀₂)(R₈₀₃),
R₈₀₁, R₈₀₂, and R₈₀₃ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, or a substituted or unsubstituted aryl group having 6 to 50 ring carbon atoms,
when a plurality of R₈₀₁ are present, the plurality of R₈₀₁ are mutually the same or different,
when a plurality of R₈₀₂ are present, the plurality of R₈₀₂ are mutually the same or different, and
when a plurality of R₈₀₃ are present, the plurality of R₈₀₃ are mutually the same or different.

In an exemplary embodiment, R₄₂₁ to R₄₂₇ and R₄₄₀ to R₄₄₈ each independently represent a hydrogen atom, a substituted or unsubstituted aryl group having 6 to 50 ring carbon atoms, or a substituted or unsubstituted heterocyclic group having 5 to 50 ring atoms.

In an exemplary embodiment, R₄₂₁ to R₄₂₇ and R₄₄₀ to R₄₄₇ are each independently selected from the group consisting of a hydrogen atom, substituted or unsubstituted aryl group having 6 to 18 ring carbon atoms, and substituted or unsubstituted heterocyclic group having 5 to 18 ring atoms.

In an exemplary embodiment, a compound represented by the formula (41-3) is a compound represented by the following formula (41-3-1).

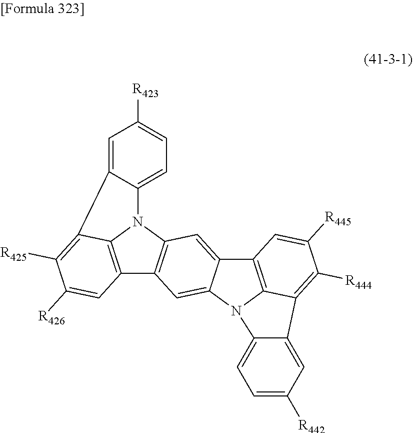

In the formula (41-3-1), R₄₂₃, R₄₂₅, R₄₂₆, R₄₄₂, R₄₄₄, and R₄₄₅ represent the same as R₄₂₃, R₄₂₅, R₄₂₆, R₄₄₂, R₄₄₄, and R₄₄₅, respectively, in the formula (41-3).

In an exemplary embodiment, a compound represented by the formula (41-3) is a compound represented by the following formula (41-3-2).

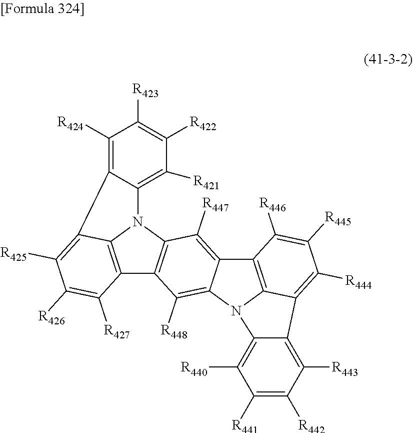

In the formula (41-3-2), R₄₂₁ to R₄₂₇ and R₄₄₀ to R₄₄₈ represent the same as R₄₂₁ to R₄₂₇ and R₄₄₀ to R₄₄₈, respectively, in the formula (41-3),
at least one of R₄₂₁ to R₄₂₇ or R₄₄₀ to R₄₄₆ represents a group represented by —N(R₉₀₆)(R₉₀₇).

In an exemplary embodiment, any two of R₄₂₁ to R₄₂₇ and R₄₄₀ to R₄₄₆ in the formula (41-3-2) represent a group represented by —N(R₉₀₆)(R₉₀₇).

In an exemplary embodiment, a compound represented by the formula (41-3-2) is a compound represented by the following formula (41-3-3).

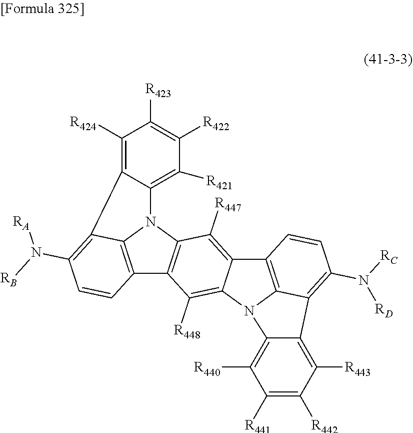

In the formula (41-3-3), R₄₂₁ to R₄₂₄, R₄₄₀ to R₄₄₃, R₄₄₇, and R₄₄₈ represent the same as R₄₂₁ to R₄₂₄, R₄₄₀ to R₄₄₃, R₄₄₇, and R₄₄₈, respectively, in the formula (41-3), and R_{A}, R_{B}, R_{C}, and R_{D} each independently represent a substituted or unsubstituted aryl group having 6 to 18 ring carbon atoms, or a substituted or unsubstituted heterocyclic group having 5 to 18 ring atoms.

In an exemplary embodiment, a compound represented by the formula (41-3-3) is a compound represented by the following formula (41-3-4).

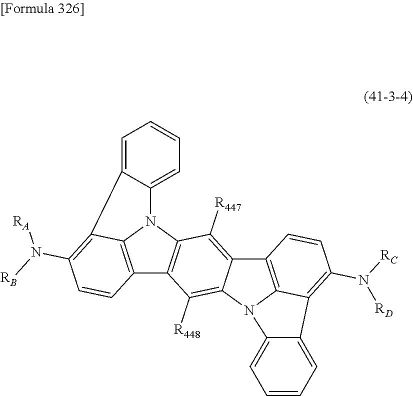

In the formula (41-3-4), R₄₄₇, R₄₄₈, R_{A}, R_{B}, R_{C}, and R_{D} represent the same as R₄₄₇, R₄₄₈, R_{A}, R_{B}, R_{C}, and R_{D}, respectively, in the formula (41-3-3).

In an exemplary embodiment, R_{A}, R_{B}, R_{C}, and R_{D} each independently represent a substituted or unsubstituted aryl group having 6 to 18 ring carbon atoms.

In an exemplary embodiment, R_{A}, R_{B}, R_{C}, and R_{D} each independently represent a substituted or unsubstituted phenyl group.

In an exemplary embodiment, R₄₄₇ and R₄₄₈ represent a hydrogen atom.

In an exemplary embodiment, a substituent for “substituted or unsubstituted” group in each of the formulae is an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁ₐ)(R₉₀₂ₐ)(R₉₀₃ₐ), —O—(R₉₀₄ₐ), —S—(R₉₀₅ₐ), —N(R₉₀₆ₐ)(R₉₀₇ₐ), a halogen atom, a cyano group, a nitro group, an unsubstituted aryl group having 6 to 50 ring carbon atoms, or an unsubstituted heterocyclic group having 5 to 50 ring atoms,
R₉₀₁ₐ to R₉₀₇ₐ each independently represent a hydrogen atom, an unsubstituted alkyl group having 1 to 50 carbon atoms, an unsubstituted aryl group having 6 to 50 ring carbon atoms, or an unsubstituted heterocyclic group having 5 to 50 ring atoms,
when two or more R₉₀₁ₐs are present, the two or more R₉₀₁ₐs are mutually the same or different,
when two or more R₉₀₂ₐs are present, the two or more R₉₀₂ₐs are mutually the same or different,
when two or more R₉₀₃ₐs are present, the two or more R₉₀₃ₐs are mutually the same or different,
when two or more R₉₀₄ₐs are present, the two or more R₉₀₄ₐs are mutually the same or different,
when two or more R₉₀₅ₐs are present, the two or more R₉₀₅ₐs are mutually the same or different,
when two or more R₉₀₆ₐs are present, the two or more R₉₀₆ₐs are mutually the same or different, and
when two or more R₉₀₇ₐs are present, the two or more R₉₀₇ₐs are mutually the same or different.

In an exemplary embodiment, a substituent for “substituted or unsubstituted” group in each of the formulae is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, a substituent for “substituted or unsubstituted” group in each of the formulae is an unsubstituted alkyl group having 1 to 18 carbon atoms, an unsubstituted aryl group having 6 to 18 ring carbon atoms, or an unsubstituted heterocyclic group having 5 to 18 ring atoms.

In the organic EL device according to the fifth exemplary embodiment, preferably, the emitting layer further contains a fluorescent compound (the compound M1), and the compound M1 is a compound that emits light with a main peak wavelength in the range of 430 nm to 480 nm.

A main peak wavelength of a compound is measured by the following method. A toluene solution of a measurement target compound at a concentration ranging from 10⁻⁶ mol/L to 10⁻⁵ mol/L is prepared and put in a quartz cell. An emission spectrum (ordinate axis: emission intensity, abscissa axis: wavelength) of the thus-obtained sample is measured at a normal temperature (300K). The emission spectrum is measurable using a spectrophotometer (machine name: F-7000) manufactured by Hitachi High-Tech Science Corporation. It should be noted that the machine for measuring the emission spectrum is not limited to the machine used herein.

A peak wavelength of the emission spectrum exhibiting the maximum luminous intensity is defined as a main peak wavelength. It should be noted that the main peak wavelength is sometimes referred to as a fluorescence main peak wavelength (FL-peak) herein.

In the organic EL device according to the fifth exemplary embodiment, when the emitting layer contains a compound represented by the formula (120) and a fluorescent compound (the compound M1), the compound represented by the formula (120) is preferably a host material (sometimes referred to as a matrix material), and the fluorescent compound (the compound M1) is preferably a dopant material (sometimes referred to as a guest material, an emitter, or a luminescent material).

In the organic EL device according to the fifth exemplary embodiment, when the emitting layer contains a compound represented by the formula (120) as a host material and a fluorescent compound (the compound M1), a singlet energy S₁ of the host material is preferably larger than a singlet energy S₁ of the fluorescent compound (the compound M1).

The singlet energy S₁ means the energy difference between the lowest singlet state and the ground state.

Singlet Energy S₁

A method for measuring the singlet energy S₁ using a solution (sometimes referred to as a solution method) may be the following method.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λedge (nm) at an intersection of the tangent and the abscissa axis is assigned to a conversion equation (F2) below to calculate the singlet energy.

S₁ [eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve fell (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

The emitting layer preferably contains no phosphorescent material (dopant material).

The emitting layer preferably do not contain a heavy metal complex and a phosphorescent rare earth metal complex. Examples of the heavy metal complex include iridium complexes, osmium complexes, and platinum complexes.

The emitting layer also preferably does not contain a metal complex.

Thickness of Emitting Layer

The emitting layer of the organic EL device according to the fifth exemplary embodiment preferably has a thickness in the range of 5 to 50 nm, more preferably 7 to 50 nm, further preferably 10 to 50 nm. An emitting layer with a thickness of 5 nm or more is easy to form, and the chromaticity of the emitting layer can be easily adjusted. When the emitting layer has a thickness of 50 nm or less, it is easy to reduce an increase in drive voltage.

Content Ratios of Compounds in Emitting Layer

When the emitting layer contains the compound according to the first exemplary embodiment (the compound represented by the formula (12X)) as a host material and a fluorescent compound (the compound M1), the content ratios of the compound according to the first exemplary embodiment and the fluorescent compound (the compound M1) in the emitting layer are preferably in the following ranges, for example.

When the emitting layer contains the compound according to the second exemplary embodiment (the compound represented by the formula (120)) as a host material and a fluorescent compound (the compound M1), the content ratios of the compound according to the second exemplary embodiment (the compound represented by the formula (120)) and the fluorescent compound (the compound M1) in the emitting layer are preferably in the following ranges, for example.

When the emitting layer contains the compound according to the third exemplary embodiment (at least one of compounds represented by one of the formulae (1) to (3)) as a host material and a fluorescent compound (the compound M1), the compound according to the third exemplary embodiment and the fluorescent compound (the compound M1) in the emitting layer are preferably in the following ranges, for example.

The content ratio of the compound according to the first exemplary embodiment, the compound according to the second exemplary embodiment, or the compound according to the third exemplary embodiment preferably ranges from 80% to 99% by mass, more preferably 90% to 99% by mass, further preferably 95% to 99% by mass.

The content ratio of the fluorescent compound (the compound M1) preferably ranges from 1% to 10% by mass, more preferably 1% to 7% by mass, further preferably 1% to 5% by mass.

The upper limit of the total content ratio of the compound according to the first exemplary embodiment, the compound according to the second exemplary embodiment, or the compound according to the third exemplary embodiment and the fluorescent compound (the compound M1) in the emitting layer is 100% by mass.

In the fifth exemplary embodiment, It is not excluded that the emitting layer contains a material other than the compound according to the first exemplary embodiment, the compound according to the second exemplary embodiment, the compound according to the third exemplary embodiment, and the fluorescent compound (the compound M1).

The emitting layer may contain one or two or more compounds according to the first exemplary embodiment (the compound represented by the formula (12X)).

The emitting layer may contain one or two or more compounds according to the second exemplary embodiment (compounds represented by the formula (120)).

The emitting layer may contain one or two or more compounds according to the third exemplary embodiment (at least one of compounds represented by one of the formulae (1) to (3)).

The emitting layer may contain one or two or more fluorescent compounds (compounds M1).

The structure of the organic EL device 1 is further described below. Reference numerals and letters may be hereinafter omitted.

Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

These materials are typically deposited by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the substance exhibiting a high hole injectability further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(Vs) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is suitably usable. The above-described substances mostly have an electron mobility of 10⁻⁶ cm²/(Vs) or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Specific examples of a compound that can be used for the electron transporting layer include the following compounds. However, the invention is not limited to the specific examples of the compound.

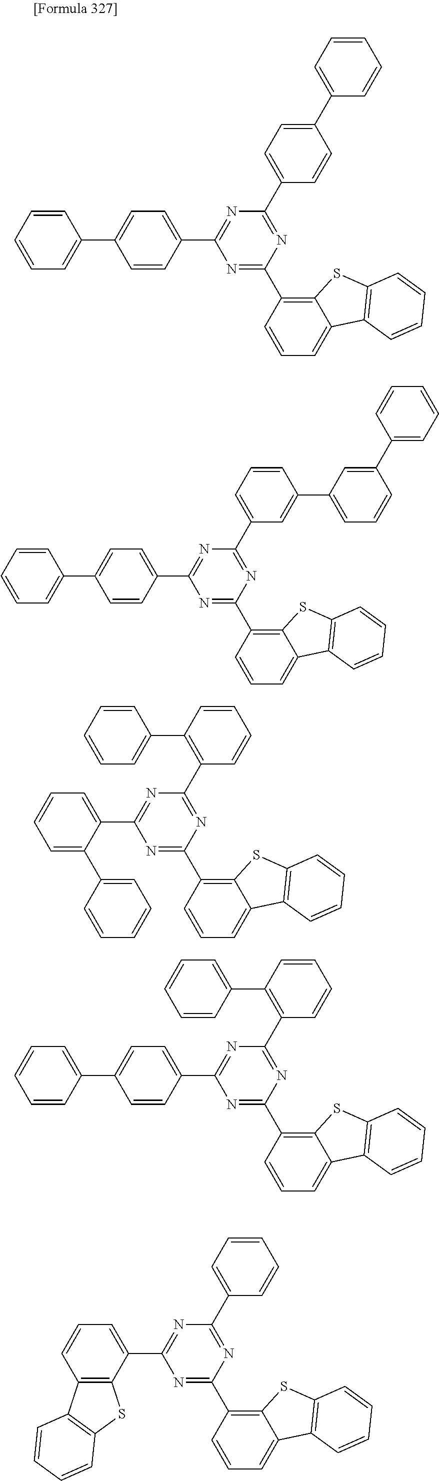

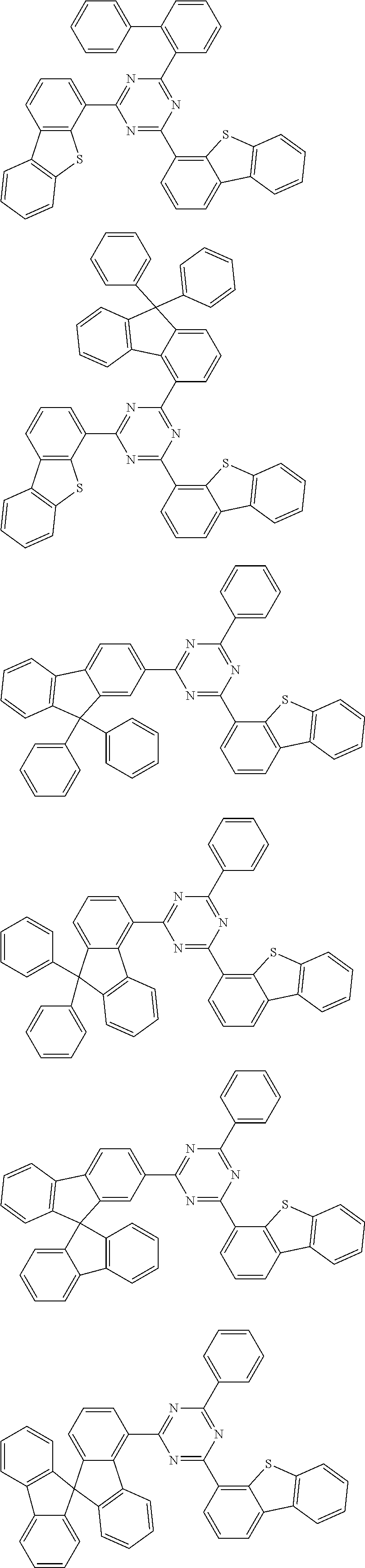

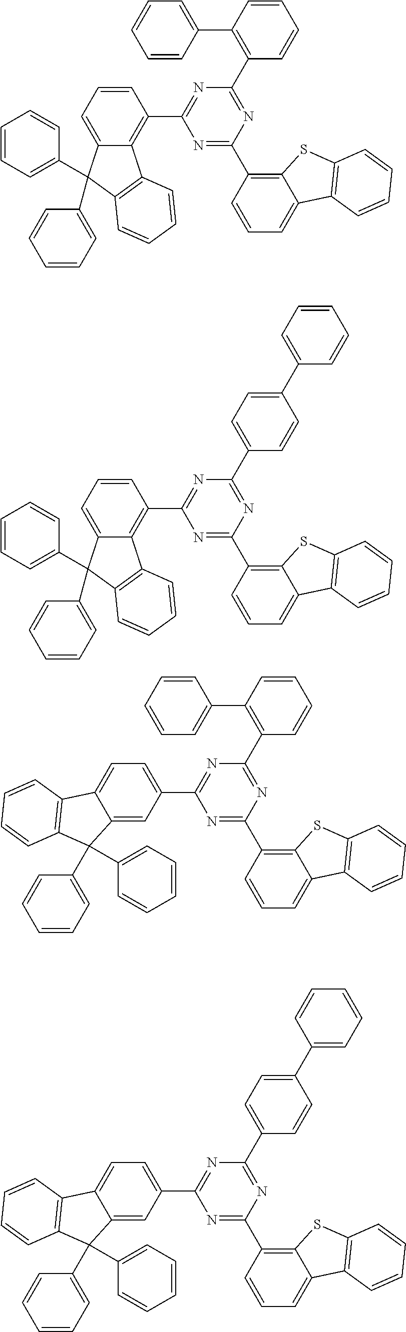

A high-molecular-weight compound may also be used for the electron transporting layer, for example, poly[(9,9-dihexylfluorene-2,7-diyl)-co-(pyridine-3,5-diyl)] (abbreviation: PF-Py) or poly[(9,9-dioctylfluorene-2,7-diyl)-co-(2,2′-bipyridine-6,6′-diyl)] (abbreviation: PF-BPy).

Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

Layer Forming Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

Film Thickness

A film thickness of each of the organic layers of the organic EL device in the exemplary embodiment is not limited unless otherwise specified in the above. In general, the thickness preferably ranges from several nanometers to 1 μm because excessively small film thickness is likely to cause defects (e.g. pin holes) and excessively large thickness leads to the necessity of applying high voltage and consequent reduction in efficiency.

The fifth exemplary embodiment can provide an organic electroluminescence device with improved luminous efficiency.

### Sixth Exemplary Embodiment

### Organic Electroluminescence Device

An organic electroluminescence device according to a sixth exemplary embodiment includes an anode, a cathode, a first emitting layer between the anode and the cathode, and a second emitting layer between the first emitting layer and the cathode. The first emitting layer has at least one group represented by the formula (11) and contains a first compound represented by the formula (1A) as a first host material, and the second emitting layer contains a second compound represented by the following formula (2) as a second host material. In the organic EL device according to the sixth exemplary embodiment, the first emitting layer is in direct contact with the second emitting layer.

In the organic EL device according to the sixth exemplary embodiment, the first emitting layer contains, as a first compound represented by the formula (1), the compound according to the first exemplary embodiment (the compound represented by the formula (12X)), the compound according to the second exemplary embodiment (the compound represented by the formula (120)), or the compound according to the third exemplary embodiment (at least one of the compounds represented by one of the formulae (1) to (3)).

The compound according to the first exemplary embodiment (the compound represented by the formula (12X)) is an embodiment of the first compound.

The compound according to the second exemplary embodiment (the compound represented by the formula (120)) is an embodiment of the first compound.

The compound according to the third exemplary embodiment (the compound represented by the formula (1), (2), or (3)) is an embodiment of the first compound.

Thus, the first emitting layer contains, as a first host material, the compound according to the first exemplary embodiment, the compound according to the second exemplary embodiment, or the compound according to the third exemplary embodiment.

In the organic EL device according to the sixth exemplary embodiment, the first emitting layer contains the compound according to the first exemplary embodiment (the compound represented by the formula (12X)) as a first host material, the second emitting layer contains a second compound represented by the following formula (2) as a second host material, and the first emitting layer is in direct contact with the second emitting layer.

In the organic EL device according to the sixth exemplary embodiment, the first emitting layer contains the compound according to the second exemplary embodiment (the compound represented by the formula (120)) as a first host material, the second emitting layer contains a second compound represented by the following formula (2) as a second host material, and the first emitting layer is in direct contact with the second emitting layer.

In the organic EL device according to the sixth exemplary embodiment, the first emitting layer contains the compound according to the third exemplary embodiment (at least one of compounds represented by one of the formulae (1) to (3)) as a first host material, the second emitting layer contains a second compound represented by the following formula (2) as a second host material, and the first emitting layer is in direct contact with the second emitting layer.

Herein, the “host material” refers to, for instance, a material that accounts for “50 mass % or more of the layer.” Accordingly, for instance, the first emitting layer contains 50 mass % or more of the first compound represented by the formula (1A) below with respect to a total mass of the first emitting layer. The second emitting layer contains 50 mass % or more of the second compound represented by the formula (2) below with respect to a total mass of the second emitting layer.

Emission Wavelength of Organic EL Device

The organic electroluminescence device according to the sixth exemplary embodiment preferably emits light with a main peak wavelength in the range of 430 nm to 480 nm when driven.

The main peak wavelength of light emitted from an organic EL device driven is measured as described below. Voltage is applied on the organic EL devices such that a current density becomes 10 mA/cm², where spectral radiance spectrum is measured by a spectroradiometer CS-2000 (manufactured by Konica Minolta, Inc.). A peak wavelength of an emission spectrum, at which the luminous intensity of the resultant spectral radiance spectrum is at the maximum, is measured and defined as the main peak wavelength (unit: nm).

The organic EL device according to the sixth exemplary embodiment may have at least one organic layer in addition to the first emitting layer and the second emitting layer. The organic layer may be at least one layer selected from the group consisting of a hole injecting layer, a hole transporting layer, an emitting layer, an electron injecting layer, an electron transporting layer, a hole blocking layer, and an electron blocking layer.

In the organic EL device according to the sixth exemplary embodiment, the organic layer may consist of the first emitting layer and the second emitting layer or may further have at least one layer selected from the group consisting of a hole injecting layer, a hole transporting layer, an electron injecting layer, an electron transporting layer, a hole blocking layer, an electron blocking layer, and the like.

The organic EL device according to the sixth exemplary embodiment preferably has a hole transporting layer between the anode and the first emitting layer.

The organic EL device according to the sixth exemplary embodiment preferably has an electron transporting layer between the cathode and the second emitting layer.

The sixth exemplary embodiment can provide an organic electroluminescence device with improved luminous efficiency.

In the organic EL device according to the sixth exemplary embodiment, the first emitting layer containing the first compound (the compound according to the first exemplary embodiment, the compound according to the second exemplary embodiment, or the compound according to the third exemplary embodiment) as a first host material is in direct contact with the second emitting layer containing the second compound represented by the following formula (2) as a second host material. The first emitting layer and the second emitting layer layered in this manner can effectively utilize singlet excitons and triplet excitons generated and consequently can improve the luminous efficiency of the organic EL device.

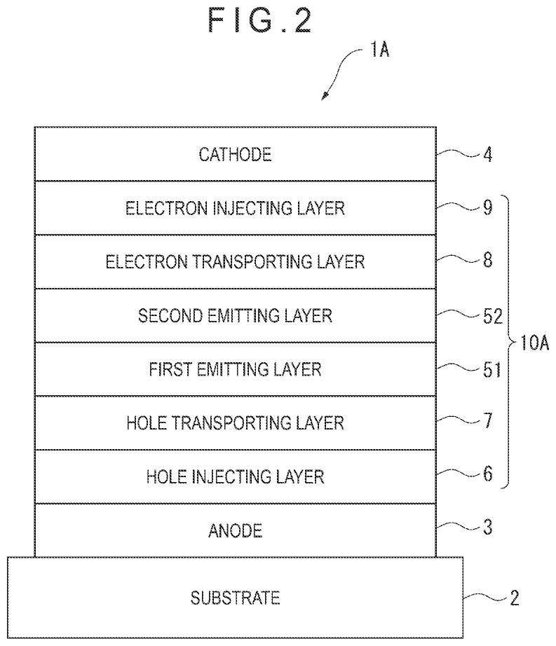

FIG. 2 is a schematic view of an exemplary arrangement of the organic EL device according to the sixth exemplary embodiment.

An organic EL device 1A includes a light-transmissive substrate 2, an anode 3, a cathode 4, and an organic layer 10A between the anode 3 and the cathode 4. The organic layer 10A includes a hole injecting layer 6, a hole transporting layer 7, a first emitting layer 51, a second emitting layer 52, an electron transporting layer 8, and an electron injecting layer 9 layered in this order on the anode 3. The first emitting layer 51 is in direct contact with the second emitting layer 52.

Second Compound

In the organic EL device according to the sixth exemplary embodiment, the second compound is a compound represented by the following formula (2).

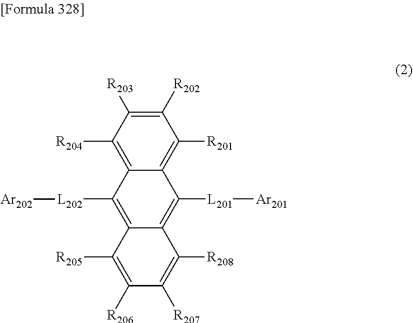

In the formula (2),
R₂₀₁ to R₂₀₈ each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms,
L₂₀₁ and L₂₀₂ each independently represent a single bond, a substituted or unsubstituted arylene group having 6 to 50 ring carbon atoms, or a substituted or unsubstituted divalent heterocyclic group having 5 to 50 ring atoms, and
Ar₂₀₁ and Ar₂₀₂ each independently represent a substituted or unsubstituted aryl group having 6 to 50 ring carbon atoms, or a substituted or unsubstituted heterocyclic group having 5 to 50 ring atoms.

In the second compound according to the sixth exemplary embodiment, R₉₀₁, R₉₀₂, R₉₀₃, R₉₀₄, R₉₀₅, R₉₀₆, R₉₀₇, R₈₀₁, and R₈₀₂ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,
when a plurality of R₉₀₁ are present, the plurality of R₉₀₁ are mutually the same or different,
when a plurality of R₉₀₂ are present, the plurality of R₉₀₂ are mutually the same or different,
when a plurality of R₉₀₃ are present, the plurality of R₉₀₃ are mutually the same or different,
when a plurality of R₉₀₄ are present, the plurality of R₉₀₄ are mutually the same or different,
when a plurality of R₉₀₅ are present, the plurality of R₉₀₅ are mutually the same or different,
when a plurality of R₉₀₆ are present, the plurality of R₉₀₆ are mutually the same or different,
when a plurality of R₉₀₇ are present, the plurality of R₉₀₇ are mutually the same or different,
when a plurality of R₈₀₁ are present, the plurality of R₈₀₁ are mutually the same or different, and
when a plurality of R₈₀₂ are present, the plurality of R₈₀₂ are mutually the same or different.

In the organic EL device according to the sixth exemplary embodiment, it is preferable that R₂₀₁ to R₂₀₈ each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, or a nitro group,
L₂₀₁ and L₂₀₂ each independently represent a single bond, a substituted or unsubstituted arylene group having 6 to 50 ring carbon atoms, or a substituted or unsubstituted divalent heterocyclic group having 5 to 50 ring atoms, and
Ar₂₀₁ and Ar₂₀₂ each independently represent a substituted or unsubstituted aryl group having 6 to 50 ring carbon atoms, or a substituted or unsubstituted heterocyclic group having 5 to 50 ring atoms.

In the organic EL device according to the sixth exemplary embodiment, it is preferable that L₂₀₁ and L₂₀₂ each independently represent a single bond, or a substituted or unsubstituted arylene group having 6 to 50 ring carbon atoms, and Ar₂₀₁ and Ar₂₀₂ each independently represent a substituted or unsubstituted aryl group having 6 to 50 ring carbon atoms.

In the organic EL device according to the sixth exemplary embodiment, Ar₂₀₁ and Ar₂₀₂ preferably each independently represent a phenyl group, a naphthyl group, a phenanthryl group, a biphenyl group, a terphenyl group, a diphenylfluorenyl group, a dimethylfluorenyl group, a benzodiphenylfluorenyl group, a benzodimethylfluorenyl group, a dibenzofuranyl group, a dibenzothienyl group, a naphthobenzofuranyl group, or a naphthobenzothienyl group.

In the organic EL device according to the sixth exemplary embodiment, the second compound represented by the formula (2) is preferably a compound represented by the formula (201), the formula (202), the formula (203), the formula (204), the formula (205), the formula (206), the formula (207), the formula (208), or the formula (209) described below.

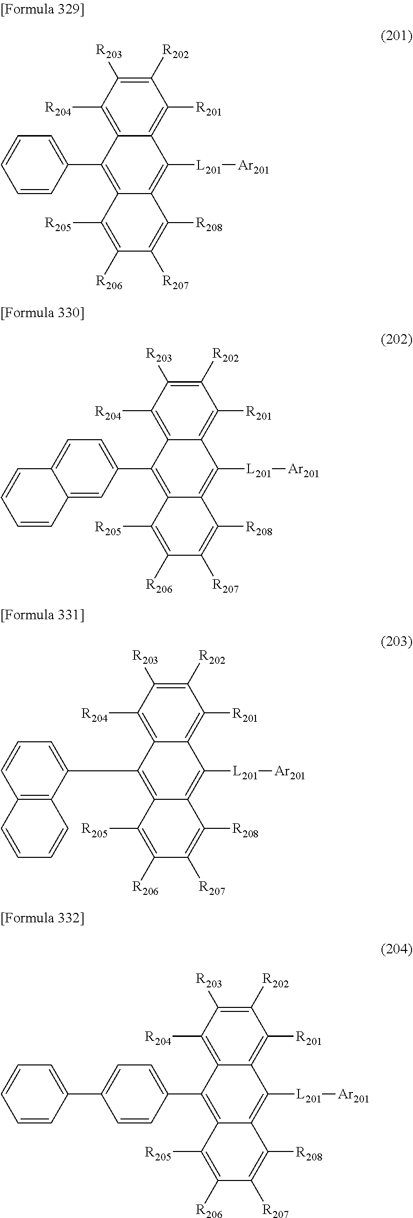

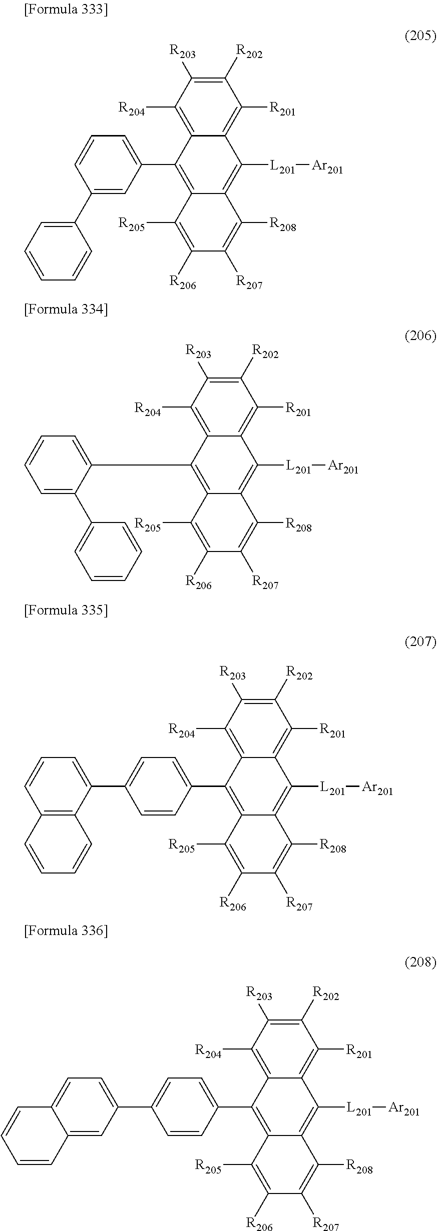

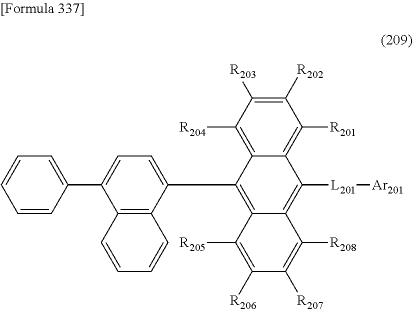

In the formula (201) to (209), L₂₀₁ and Ar₂₀₁ represent the same as L₂₀₁ and Ar₂₀₁, respectively, in the formula (2), and R₂₀₁ to R₂₀₈ represent the same as R₂₀₁ to R₂₀₈, respectively, in the formula (2).

The second compound represented by the formula (2) is also preferably a compound represented by the formula (221), the formula (222), the formula (223), the formula (224), the formula (225), the formula (226), the formula (227), the formula (228), or the formula (229) described below.

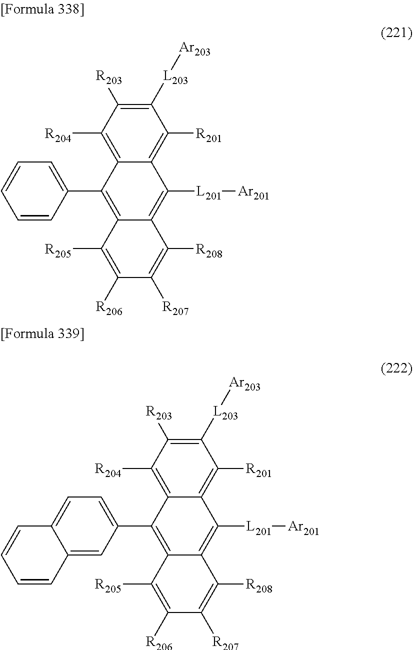

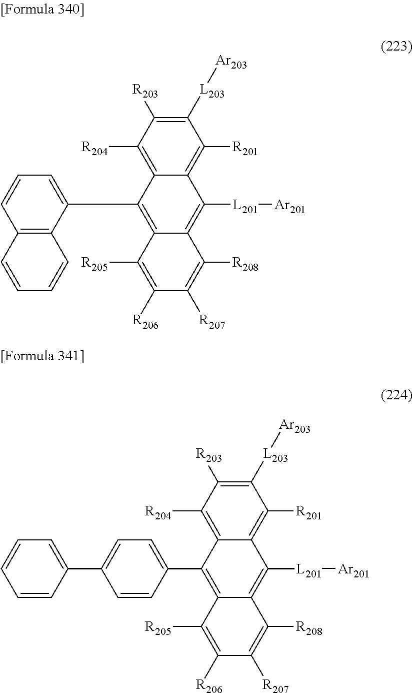

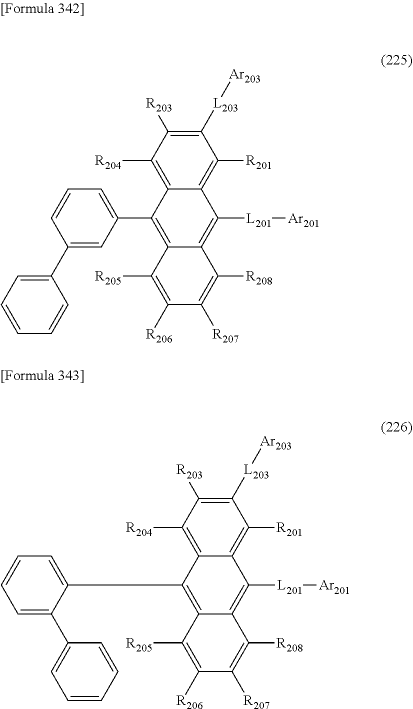

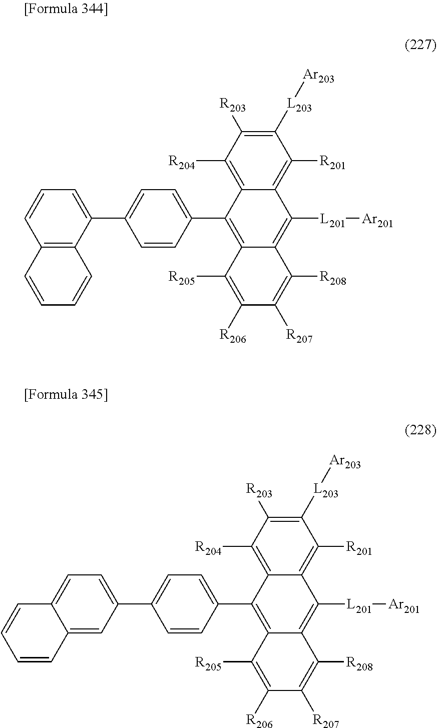

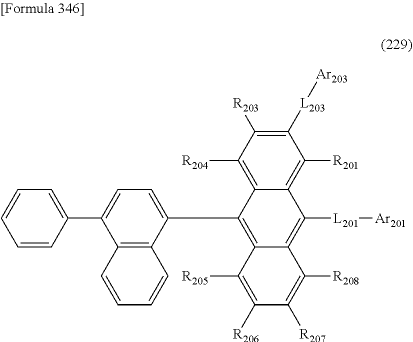

In the formula (221), the formula (222), the formula (223), the formula (224), the formula (225), the formula (226), the formula (227), the formula (228), and the formula (229),
R₂₀₁ and R₂₀₃ to R₂₀₈ represent the same as R₂₀₁ and R₂₀₃ to R₂₀₈, respectively, in the formula (2),
L₂₀₁ and Ar₂₀₁ represent the same as L₂₀₁ and Ar₂₀₁, respectively, in the formula (2),
L₂₀₃ represents the same as L₂₀₁ in the formula (2),
L₂₀₃ and L₂₀₁ are mutually the same or different,
Ar₂₀₃ represents the same as Ar₂₀₁ in the formula (2), and
Ar₂₀₃ and Ar₂₀₁ are mutually the same or different.

The second compound represented by the formula (2) is also preferably a compound represented by the formula (241), the formula (242), the formula (243), the formula (244), the formula (245), the formula (246), the formula (247), the formula (248), or the formula (249) described below.

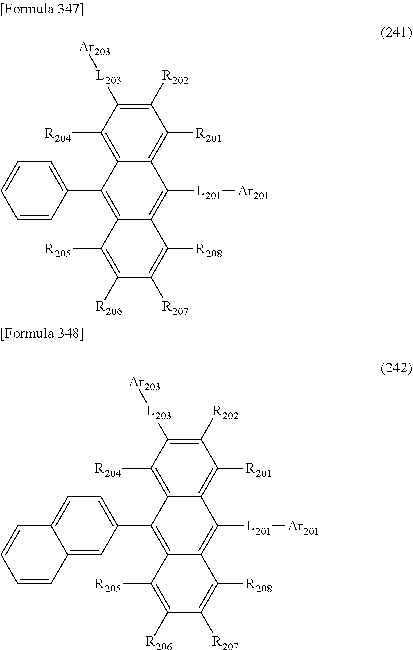

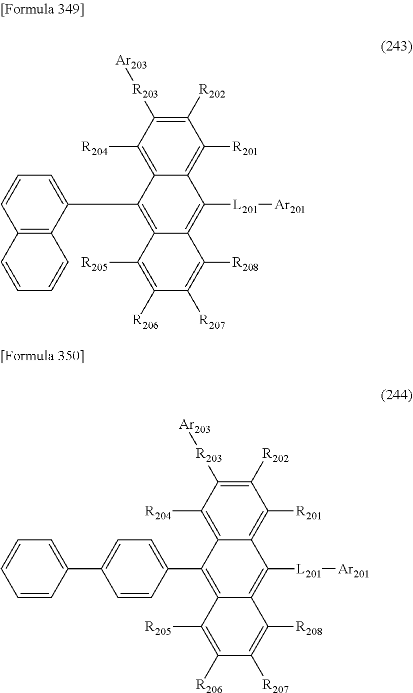

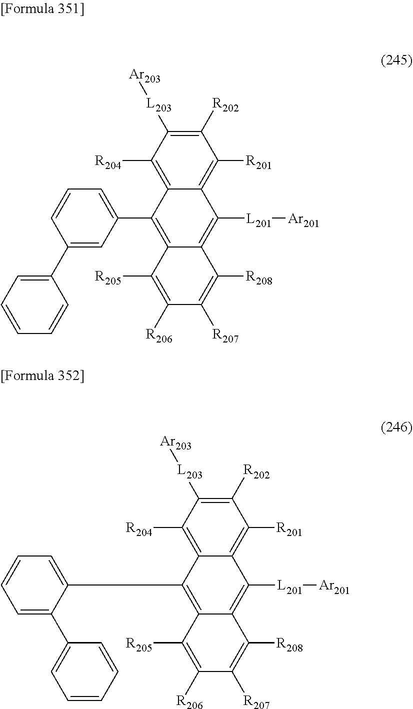

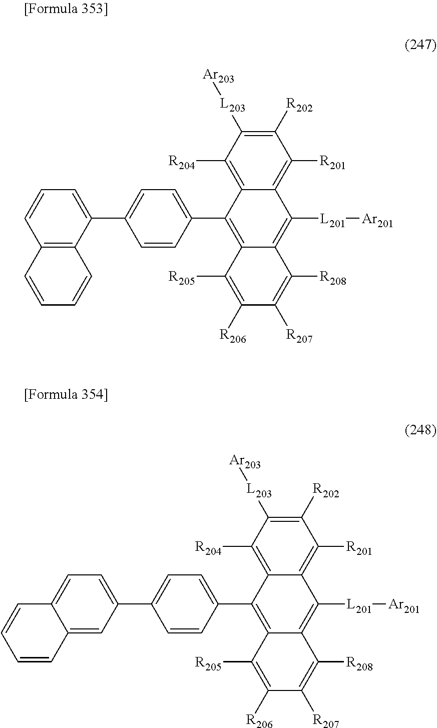

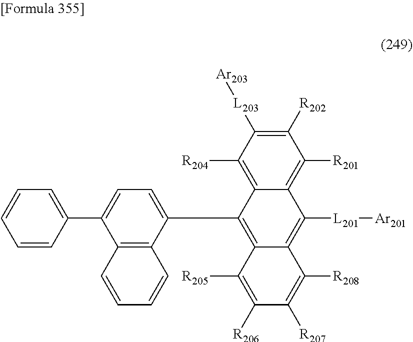

In the formula (241), the formula (242), the formula (243), the formula (244), the formula (245), the formula (246), the formula (247), the formula (248), and the formula (249),
R₂₀₁, R₂₀₂, and R₂₀₄ to R₂₀₈ represent the same as R₂₀₁, R₂₀₂, and R₂₀₄ to R₂₀₈, respectively, in the formula (2),
L₂₀₃ represents the same as L₂₀₁ in the formula (2),
L₂₀₃ and L₂₀₁ are mutually the same or different, Ar₂₀₃ represents the same as Ar₂₀₁ in the formula (2), and
Ar₂₀₃ and Ar₂₀₁ are mutually the same or different.

In the second compound represented by the formula (2), R₂₀₁ to R₂₀₈ that are not groups represented by the formula (21) preferably each independently represent a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

Preferably, L₁₀₁ represents a single bond, or an unsubstituted arylene group having 6 to 22 ring carbon atoms, and Ar₁₀₁ represents a substituted or unsubstituted aryl group having 6 to 22 ring carbon atoms.

In the organic EL device according to the sixth exemplary embodiment, in the second compound represented by the formula (2), R₂₀₁ to R₂₀₈ preferably each independently represent a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In the organic EL device according to the sixth exemplary embodiment, in the second compound represented by the formula (2), R₂₀₁ to R₂₀₈ preferably represent a hydrogen atom.

In the second compound, the groups specified to be “substituted or unsubstituted” are each preferably an “unsubstituted” group.

In the organic EL device according to the sixth exemplary embodiment, for example, Ar₂₀₁ in the second compound represented by the formula (2) represents a substituted or unsubstituted dibenzofuranyl group.

In the organic EL device according to the sixth exemplary embodiment, for example, Ar₂₀₁ in the second compound represented by the formula (2) represents an unsubstituted dibenzofuranyl group.

In the organic EL device according to the sixth exemplary embodiment, for example, the second compound represented by the formula (2) has at least one hydrogen, and at least one of the hydrogen(s) is deuterium.

In the organic EL device according to the sixth exemplary embodiment, for example, L₂₀₁ in the second compound represented by the formula (2) represents one of TEMP-63 to TEMP-68.

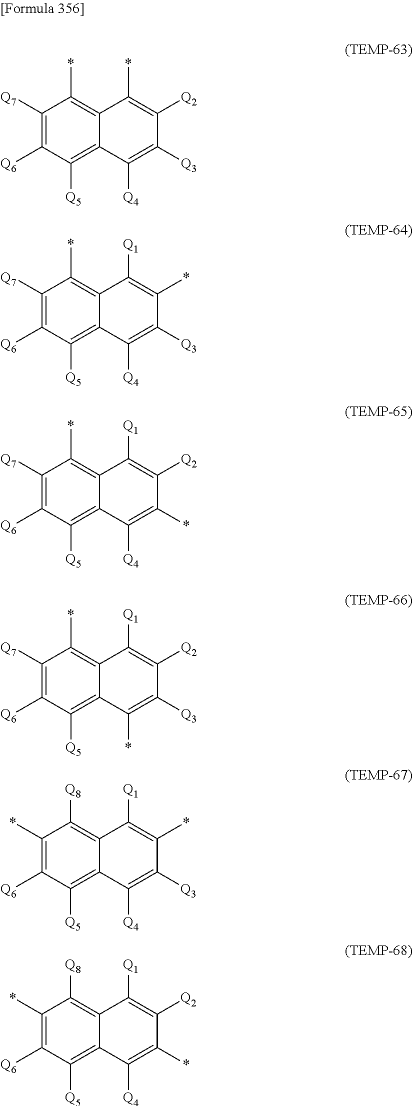

In the organic EL device according to the sixth exemplary embodiment, for example, Ar₂₀₁ in the second compound represented by the formula (2) represents at least one group selected from the group consisting of substituted or unsubstituted anthryl group, benzoanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluoranthenyl group, benzofluoranthenyl group, and perylenyl group.

In the organic EL device according to the sixth exemplary embodiment, for example, Ar₂₀₁ in the second compound represented by the formula (2) represents a substituted or unsubstituted fluorenyl group.

In the organic EL device according to the sixth exemplary embodiment, for example, Ar₂₀₁ in the second compound represented by the formula (2) represents a substituted or unsubstituted xanthenyl group.

In the organic EL device according to the sixth exemplary embodiment, for example, Ar₂₀₁ in the second compound represented by the formula (2) represents a substituted or unsubstituted benzoxanthenyl group.

Method for Producing Second Compound

The second compound can be produced by a known method. The second compound can also be produced in accordance with a known method by using a known alternative reaction and raw materials suitable for the target compound.

Specific examples of the second compound include the following compounds. However, the invention is not limited to the specific examples of the second compound.

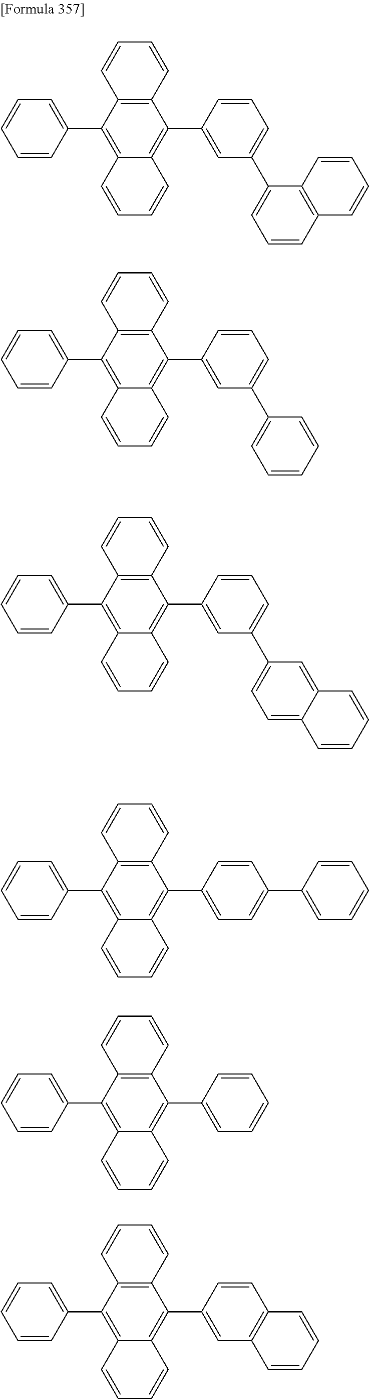

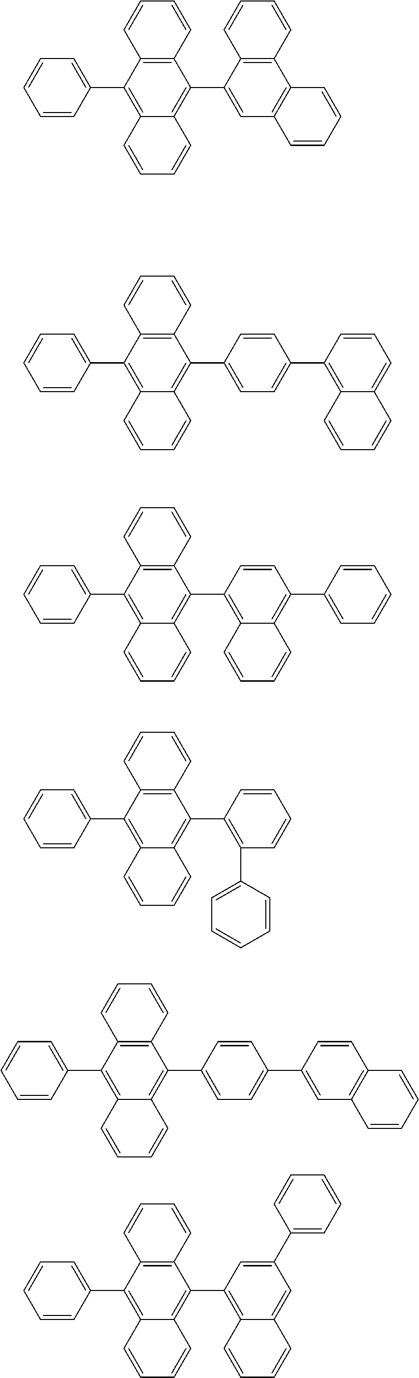

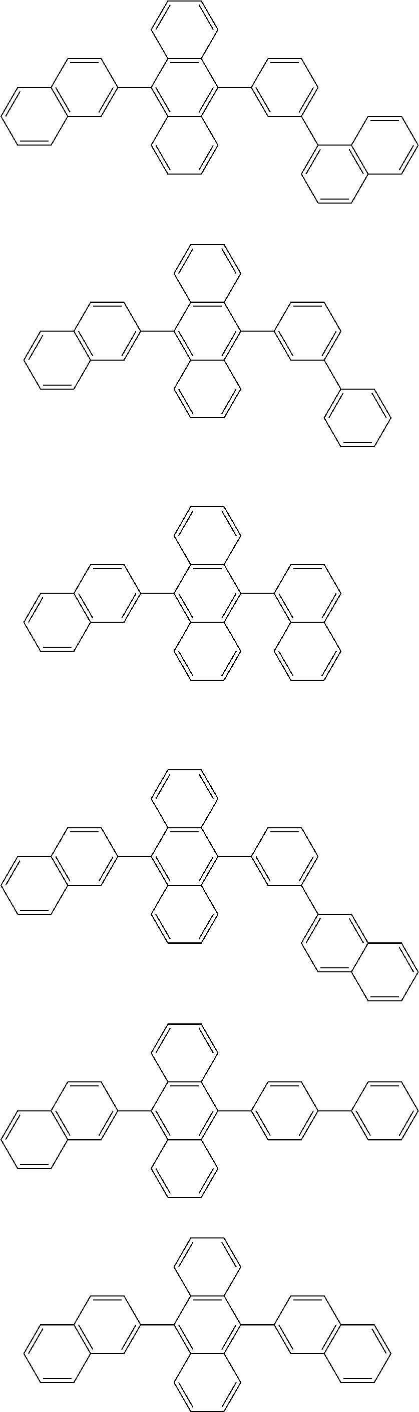

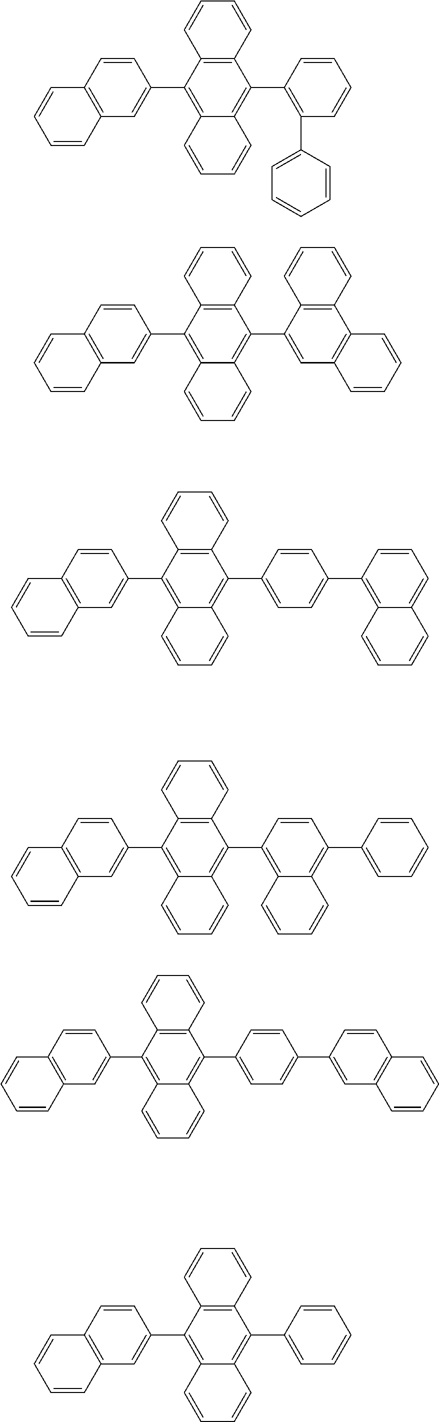

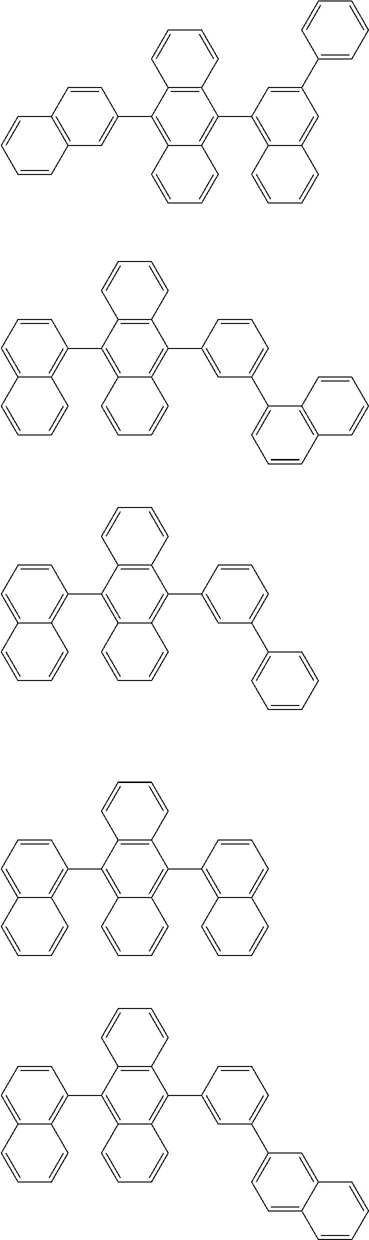

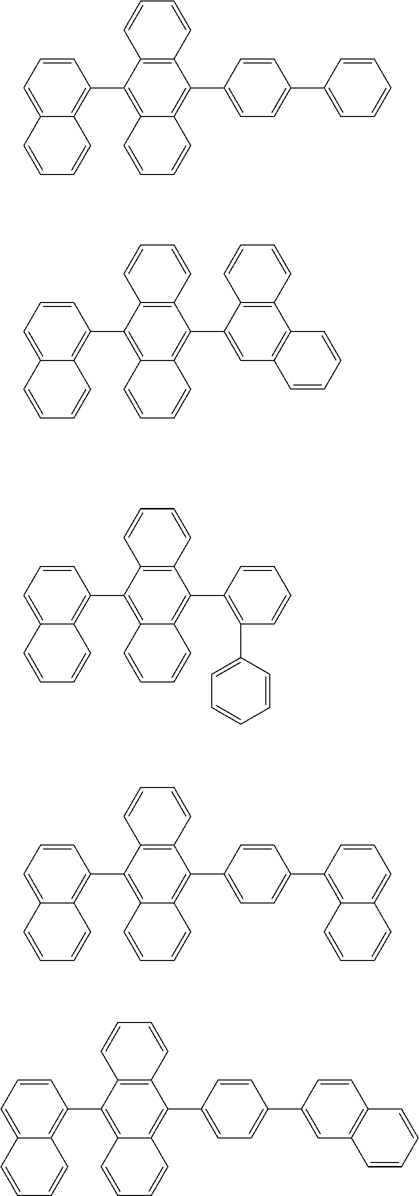

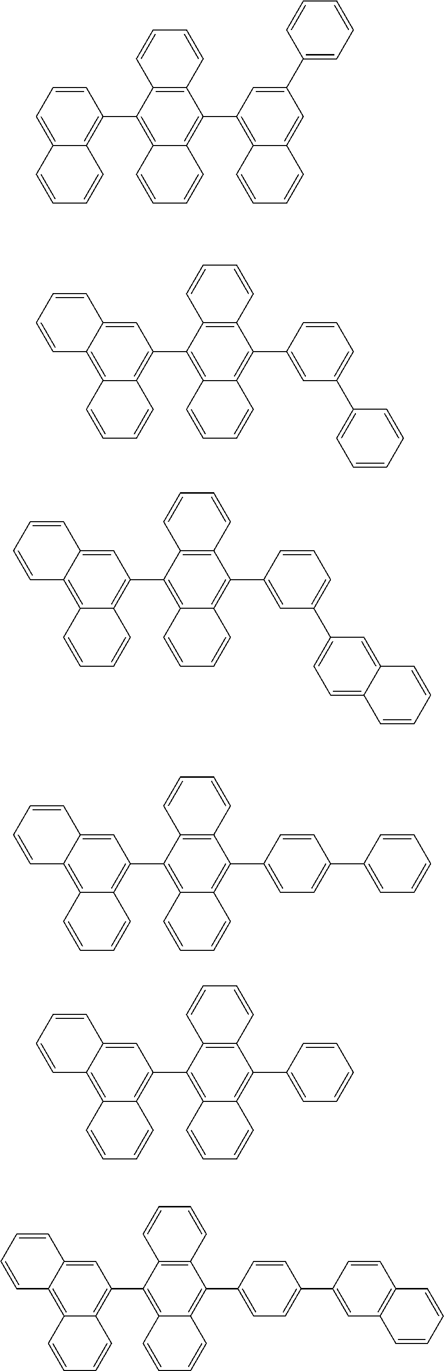

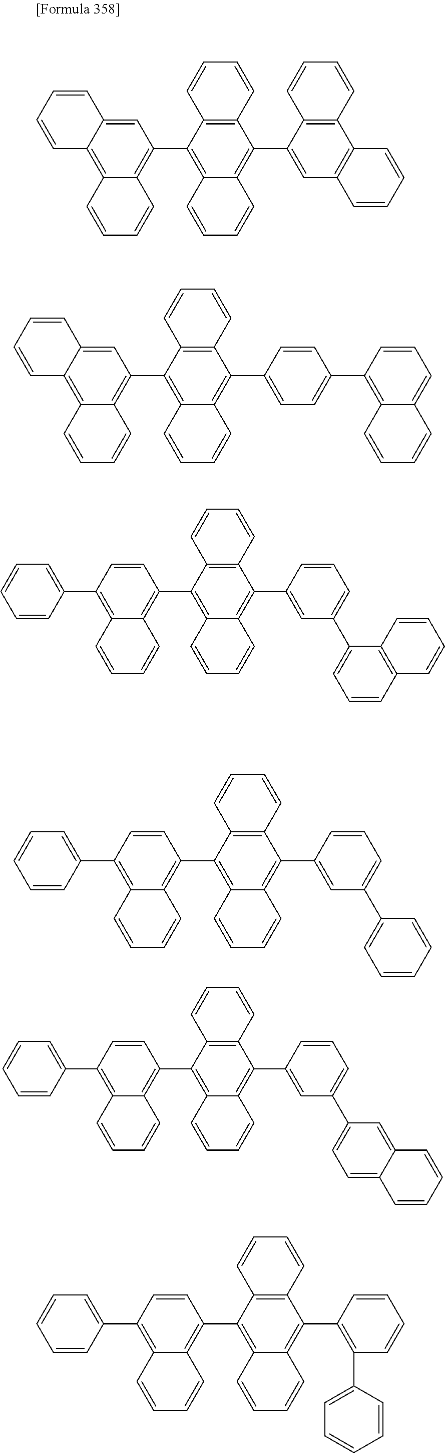

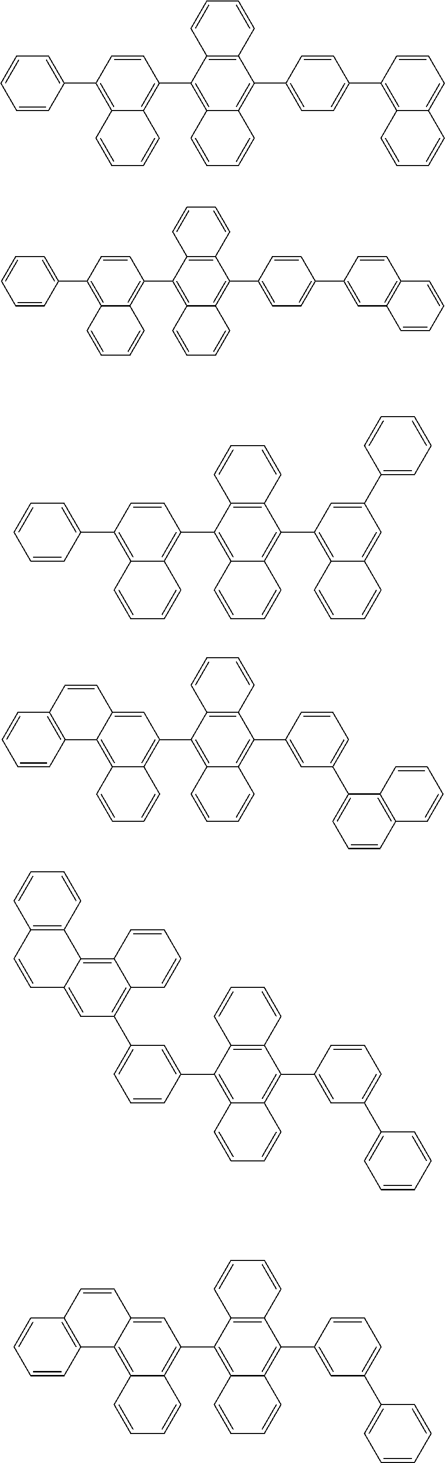

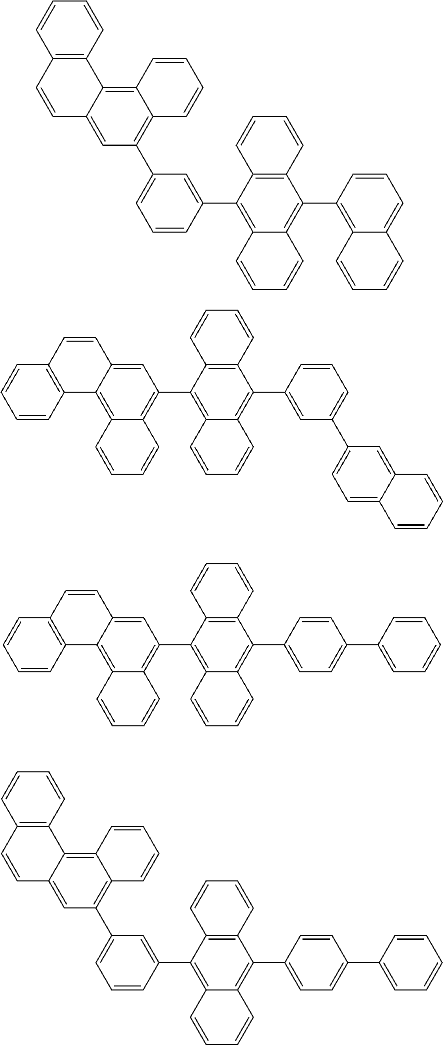

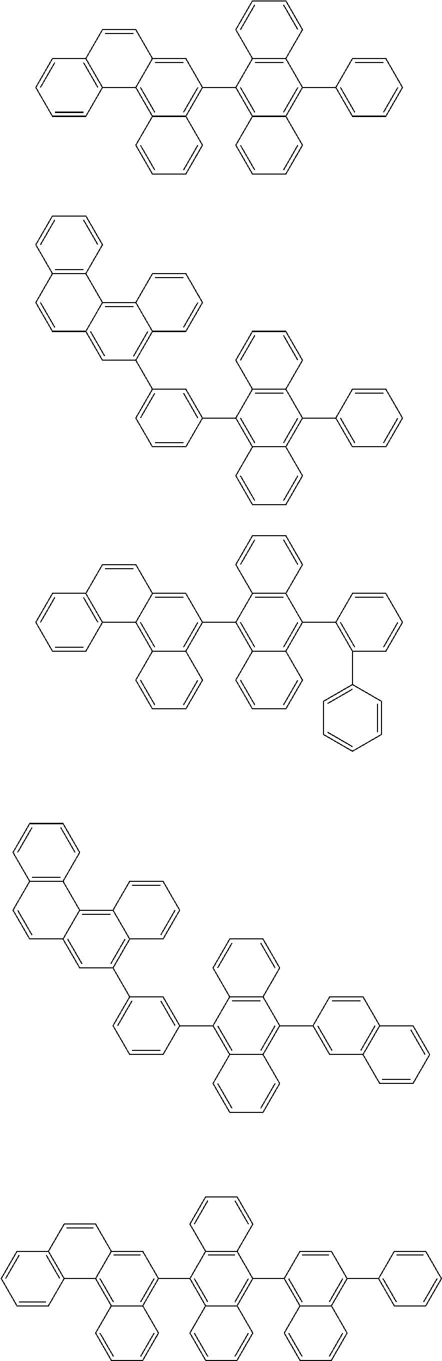

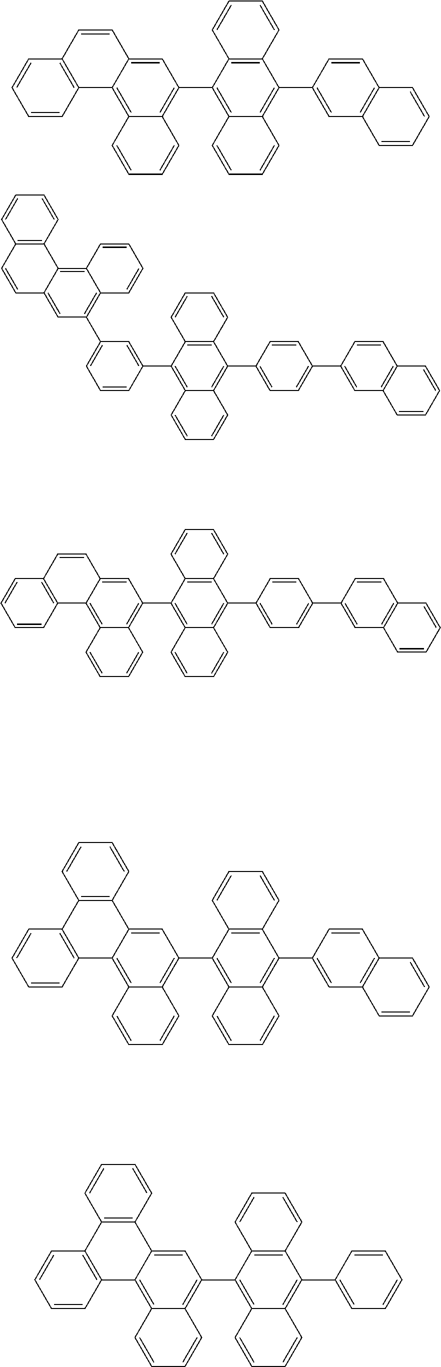

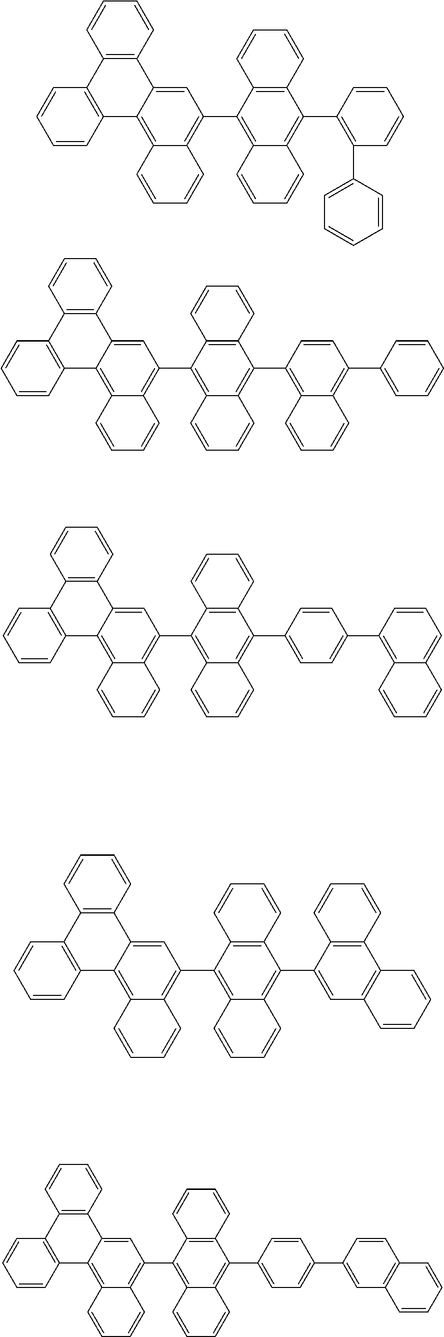

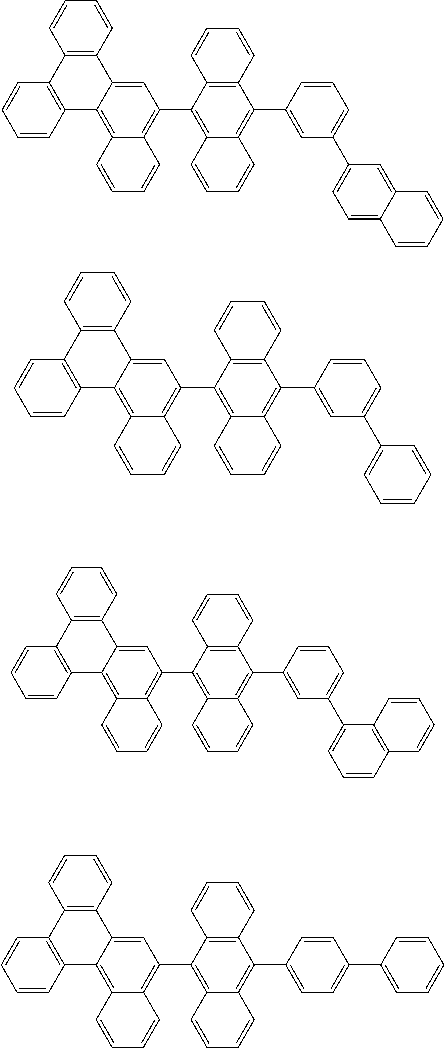

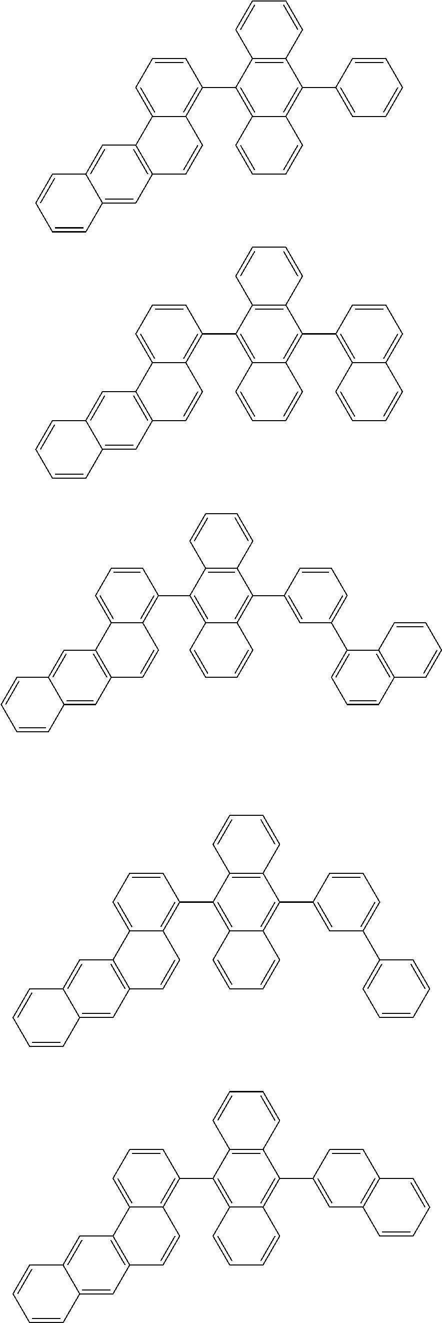

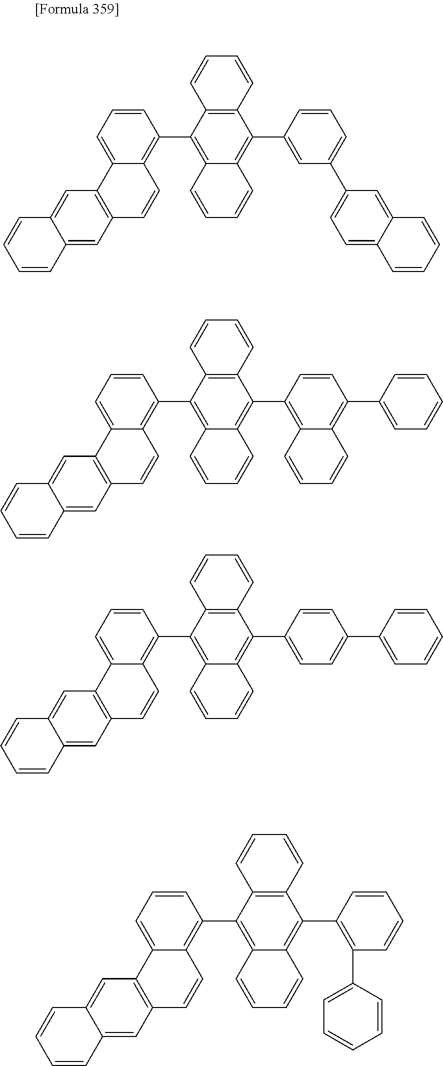

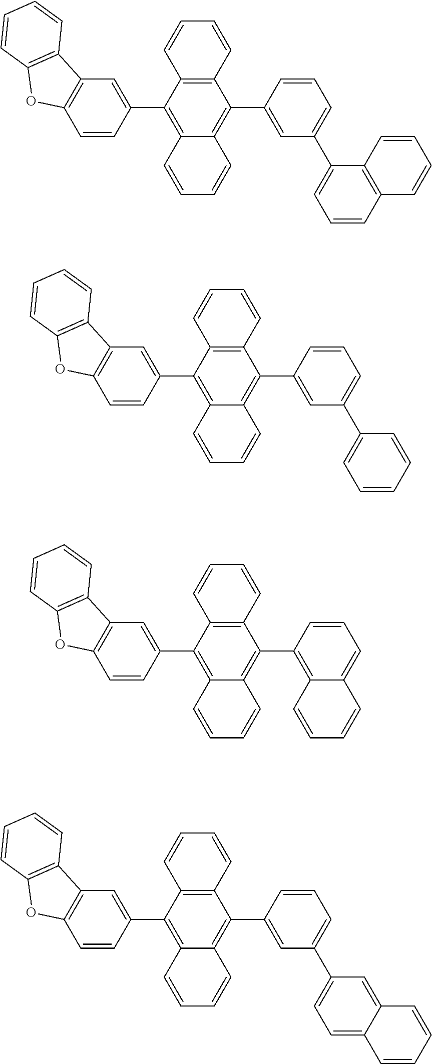 [SMILES: c1cc(-c2cccc3ccccc23)cc(-c2c3ccccc3c(-c3ccc4oc5ccccc5c4c3)c3ccccc23)c1 ; c1ccc(-c2cccc(-c3c4ccccc4c(-c4ccc5oc6ccccc6c5c4)c4ccccc34)c2)cc1 ; c1ccc2c(-c3c4ccccc4c(-c4ccc5oc6ccccc6c5c4)c4ccccc34)cccc2c1 ; c1cc(-c2ccc3ccccc3c2)cc(-c2c3ccccc3c(-c3ccc4oc5ccccc5c4c3)c3ccccc23)c1]

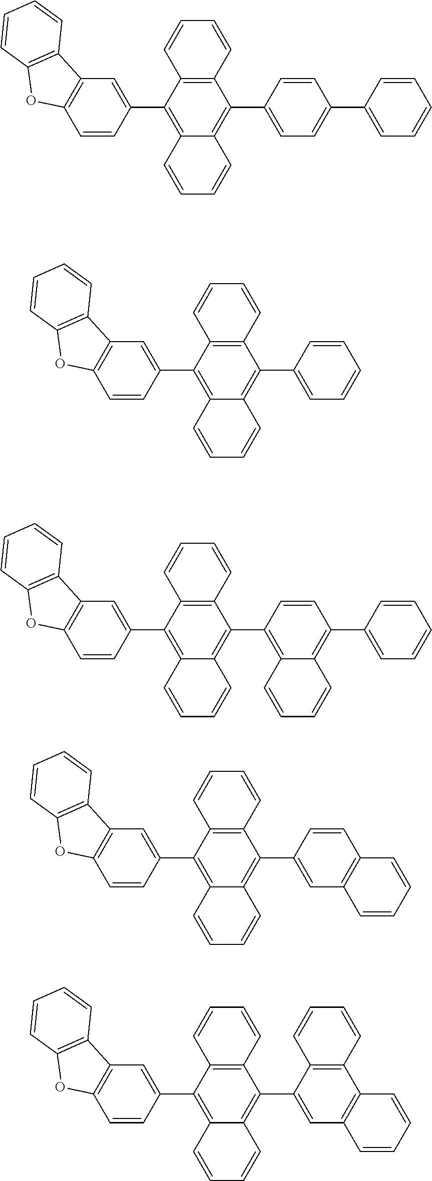 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1 ; c1ccc(-c2ccc(-c3c4ccccc4c(-c4ccc5oc6ccccc6c5c4)c4ccccc34)c3ccccc23)cc1 ; c1ccc2cc(-c3c4ccccc4c(-c4ccc5oc6ccccc6c5c4)c4ccccc34)ccc2c1 ; c1ccc2c(c1)cc(-c1c3ccccc3c(-c3ccc4oc5ccccc5c4c3)c3ccccc13)c1ccccc12]

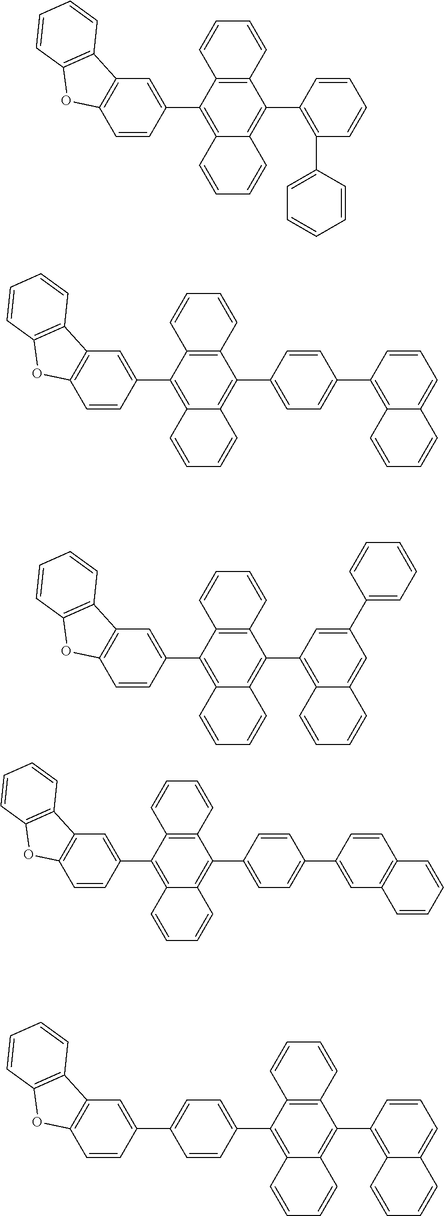

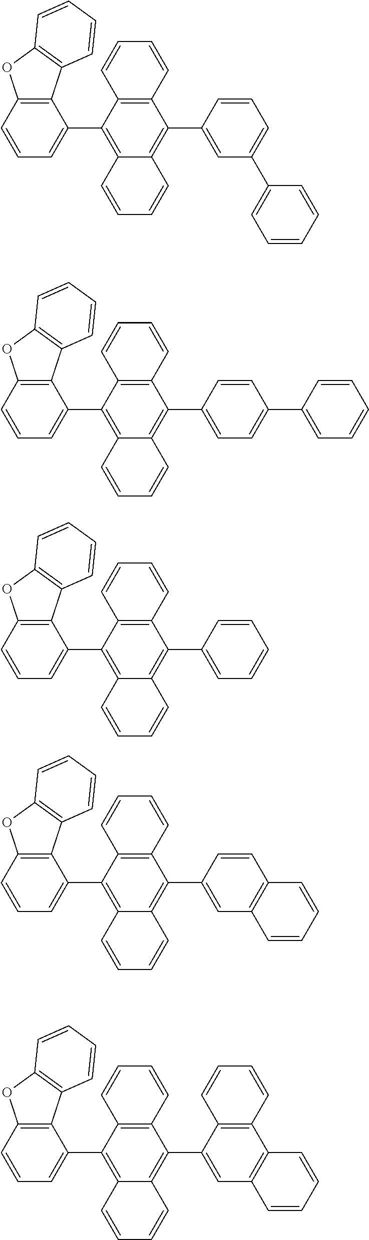

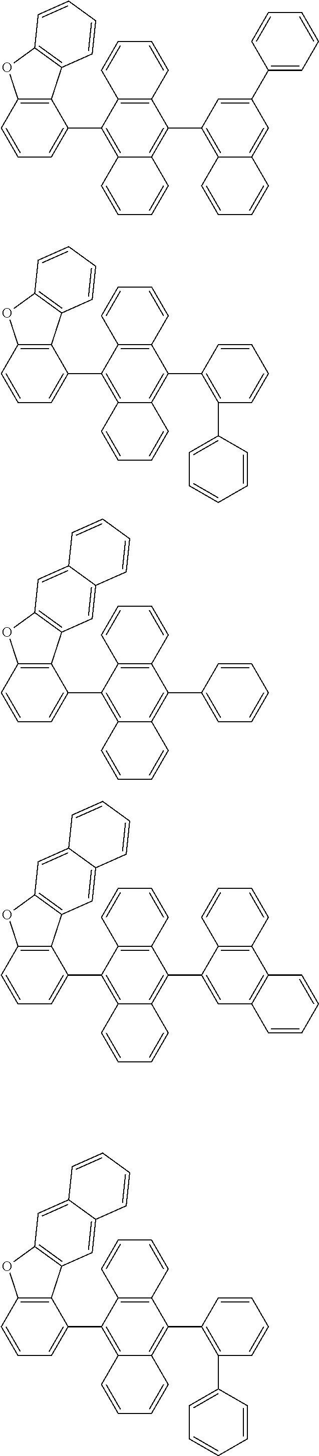

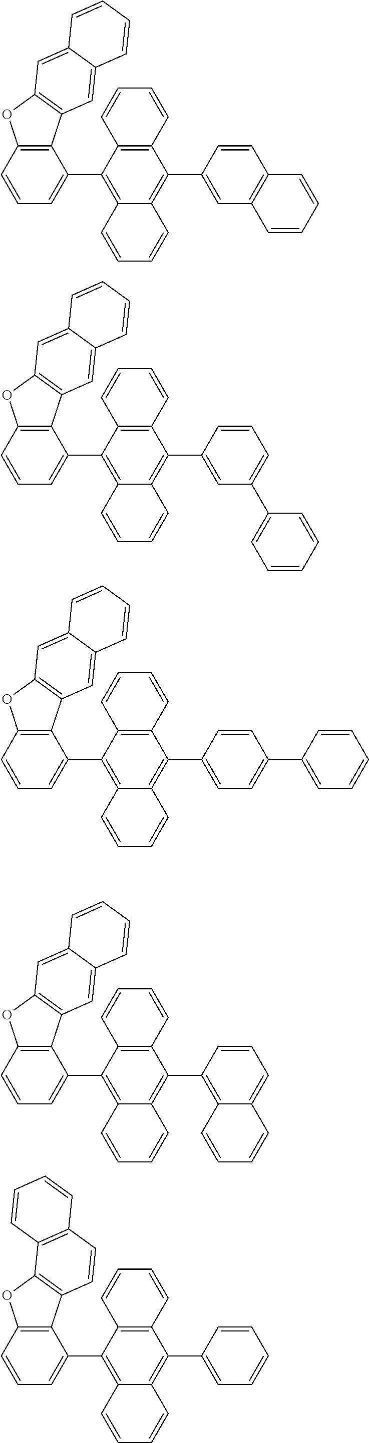

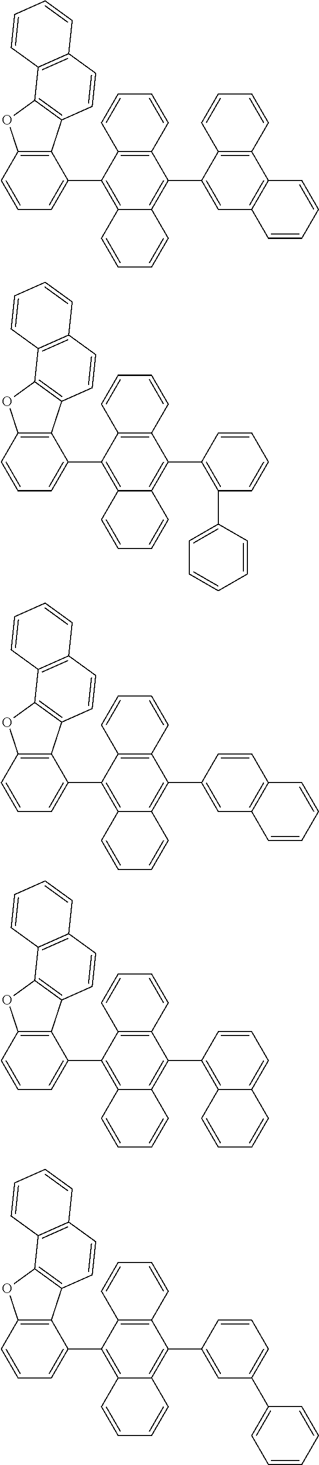 [SMILES: c1ccc2c(c1)cc(-c1c3ccccc3c(-c3cccc4oc5c6ccccc6ccc5c34)c3ccccc13)c1ccccc12 ; c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1 ; c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1]

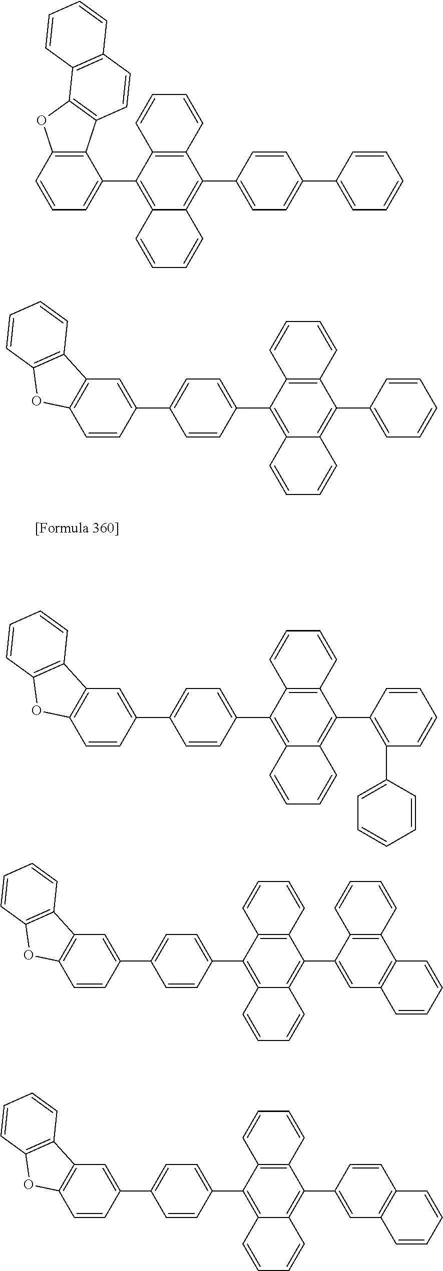

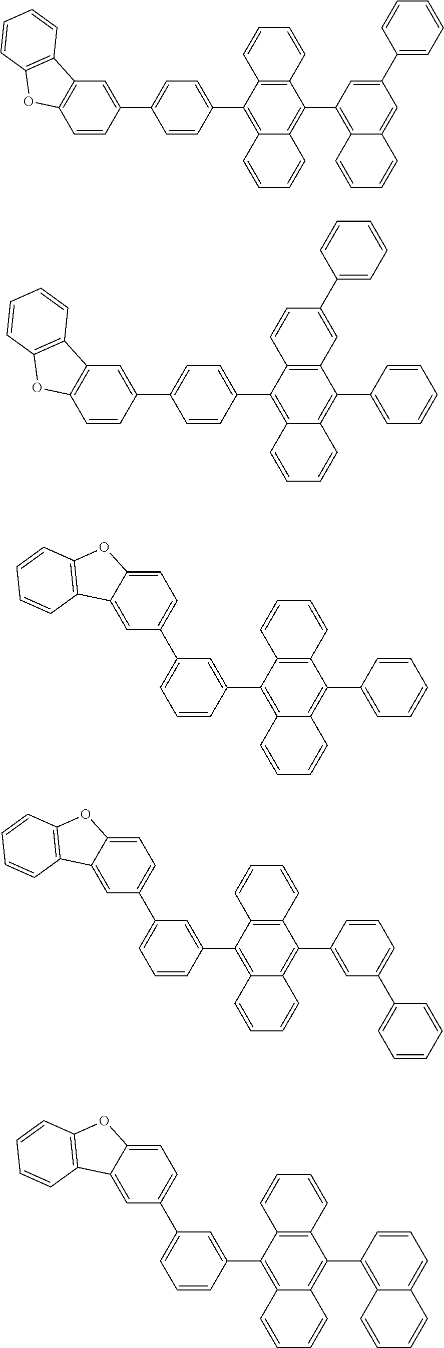

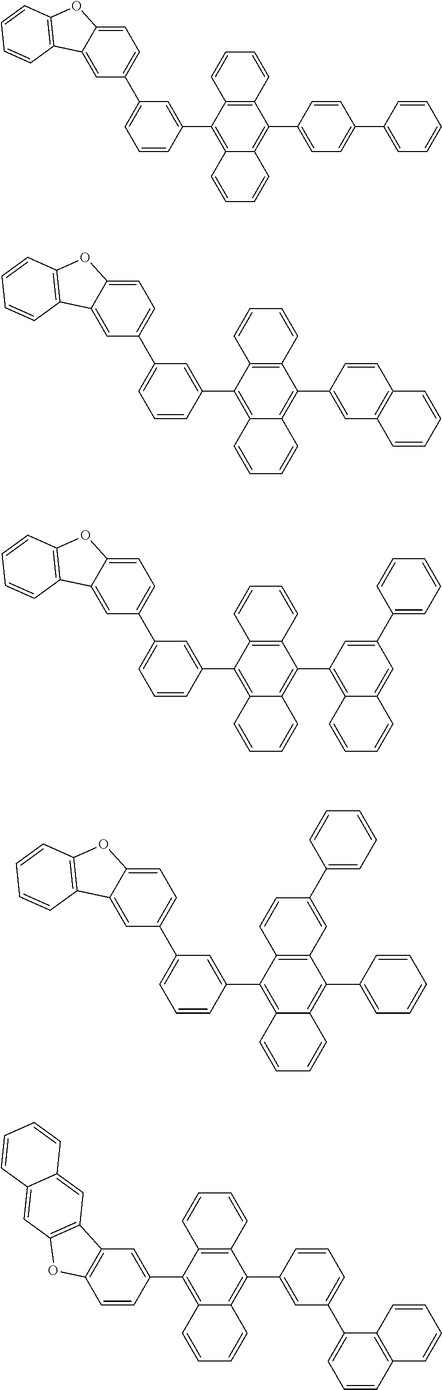 [SMILES: c1ccc(-c2ccc(-c3c4ccccc4c(-c4cccc(-c5ccc6oc7ccccc7c6c5)c4)c4ccccc34)cc2)cc1 ; c1cc(-c2ccc3oc4ccccc4c3c2)cc(-c2c3ccccc3c(-c3ccc4ccccc4c3)c3ccccc23)c1 ; c1ccc(-c2cc(-c3c4ccccc4c(-c4cccc(-c5ccc6oc7ccccc7c6c5)c4)c4ccccc34)c3ccccc3c2)cc1 ; c1ccc(-c2ccc3c(-c4cccc(-c5ccc6oc7ccccc7c6c5)c4)c4ccccc4c(-c4ccccc4)c3c2)cc1 ; c1cc(-c2cccc3ccccc23)cc(-c2c3ccccc3c(-c3ccc4oc5cc6ccccc6cc5c4c3)c3ccccc23)c1]

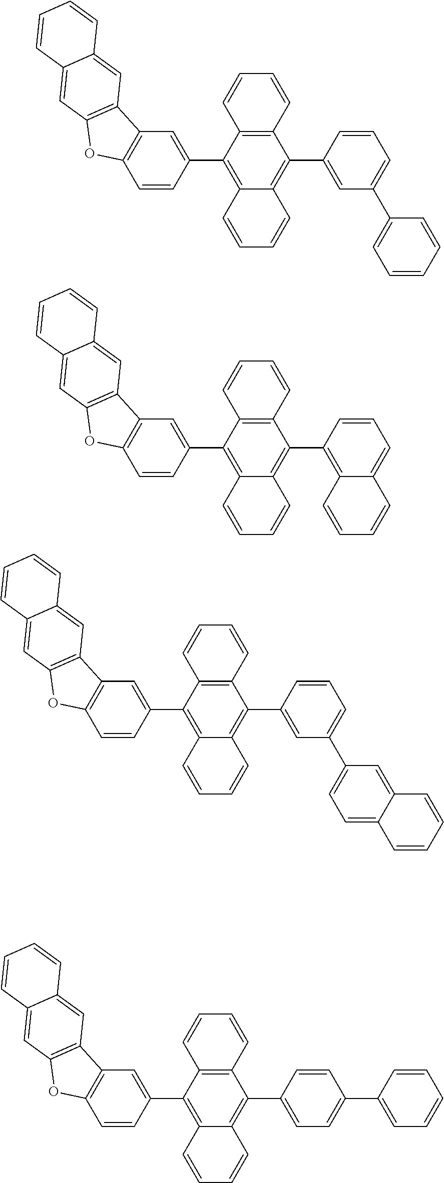 [SMILES: c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1 ; c1ccc(-c2ccc(-c3c4ccccc4c(-c4ccc5oc6cc7ccccc7cc6c5c4)c4ccccc34)cc2)cc1]

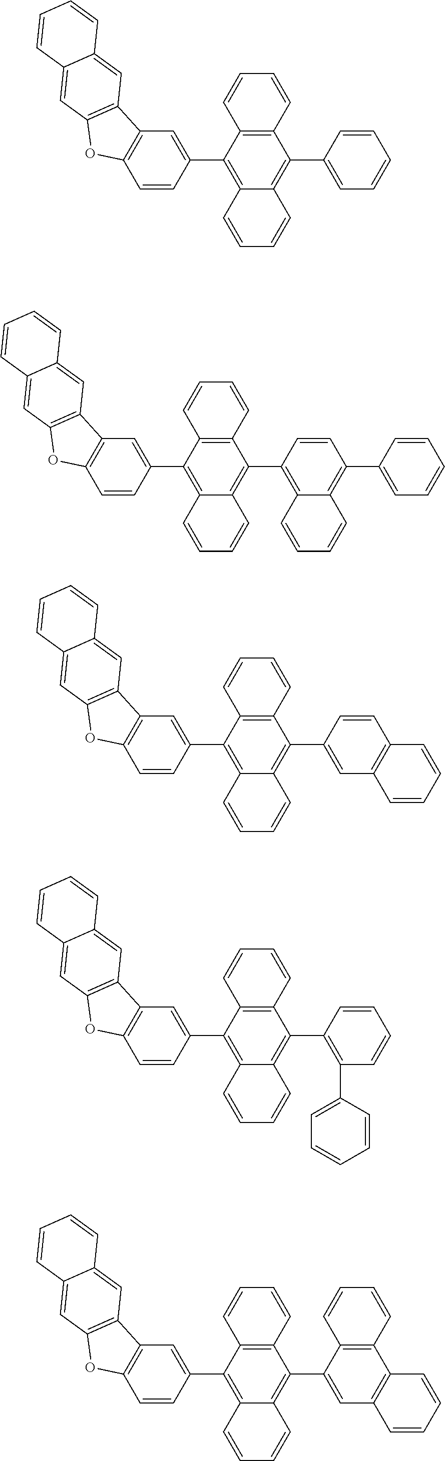 [SMILES: c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5ccccc5c5ccccc45)c4ccccc24)cc13]

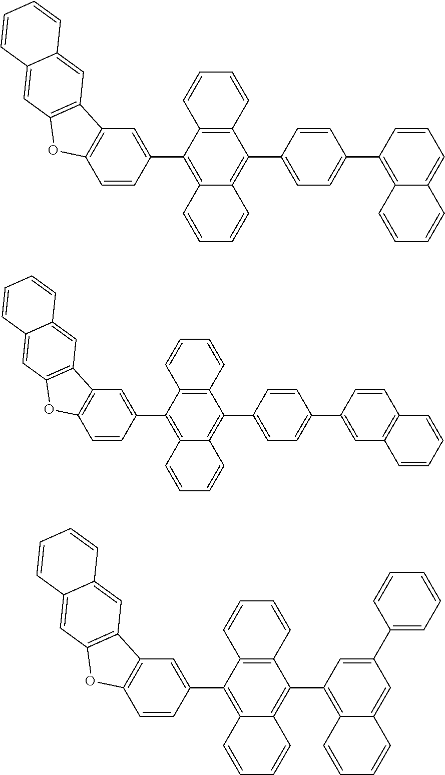 [SMILES: c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1 ; c1ccc(-c2cc(-c3c4ccccc4c(-c4ccc5oc6cc7ccccc7cc6c5c4)c4ccccc34)c3ccccc3c2)cc1]

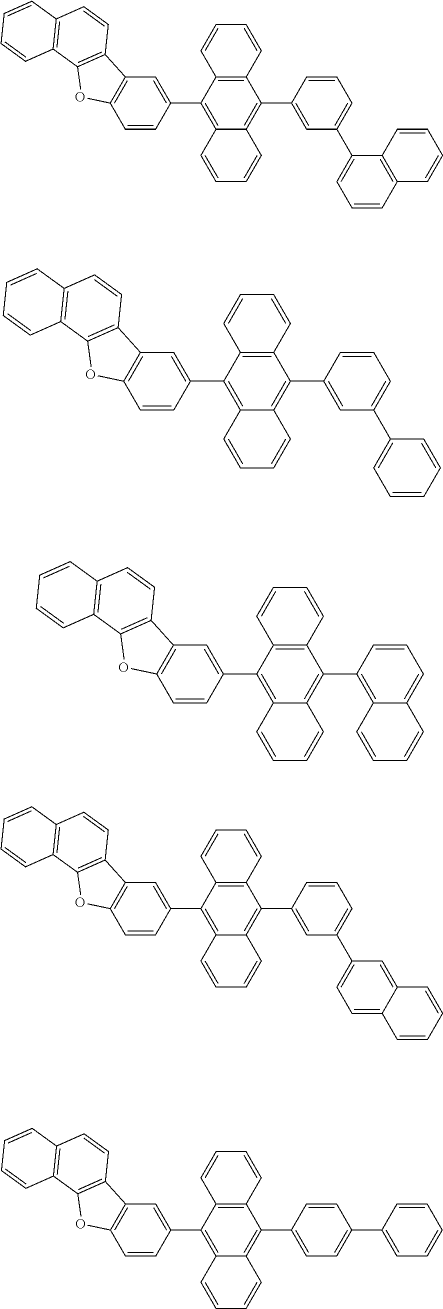 [SMILES: c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1 ; c1cc(-c2ccc3ccccc3c2)cc(-c2c3ccccc3c(-c3ccc4oc5c6ccccc6ccc5c4c3)c3ccccc23)c1 ; c1ccc(-c2ccc(-c3c4ccccc4c(-c4ccc5oc6c7ccccc7ccc6c5c4)c4ccccc34)cc2)cc1]

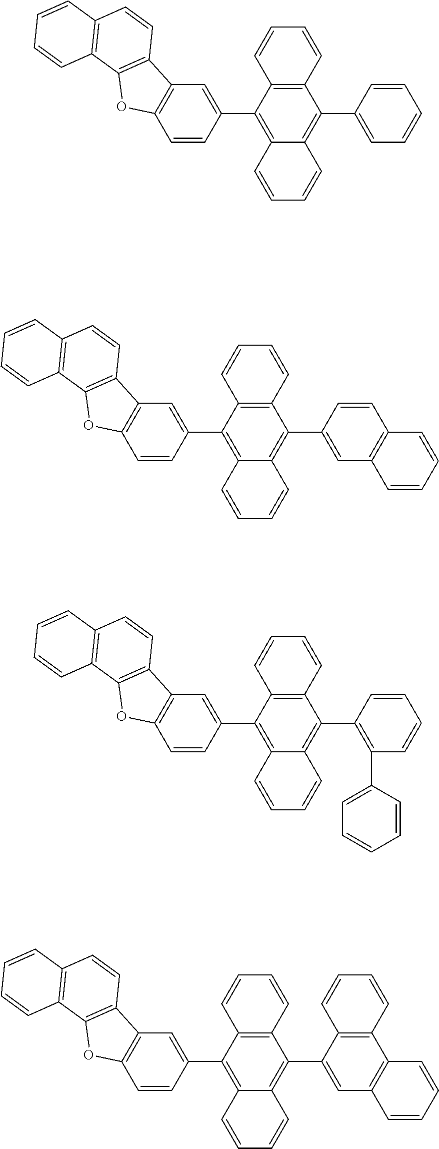 [SMILES: c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1 ; c1ccc(-c2ccccc2-c2c3ccccc3c(-c3ccc4oc5c6ccccc6ccc5c4c3)c3ccccc23)cc1 ; c1ccc2c(c1)cc(-c1c3ccccc3c(-c3ccc4oc5c6ccccc6ccc5c4c3)c3ccccc13)c1ccccc12]

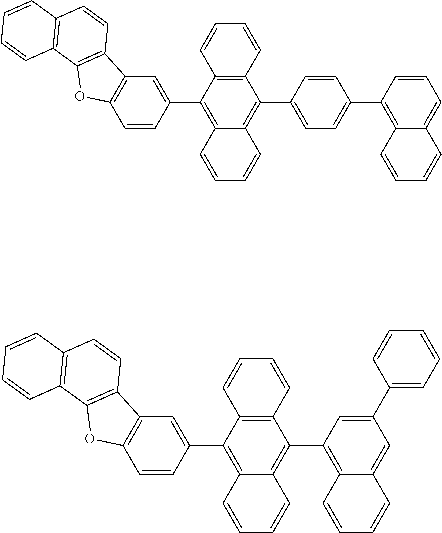 [SMILES: c1ccc2c(-c3ccc(-c4c5ccccc5c(-c5ccc6oc7c8ccccc8ccc7c6c5)c5ccccc45)cc3)cccc2c1 ; c1ccc(-c2cc(-c3c4ccccc4c(-c4ccc5oc6c7ccccc7ccc6c5c4)c4ccccc34)c3ccccc3c2)cc1]

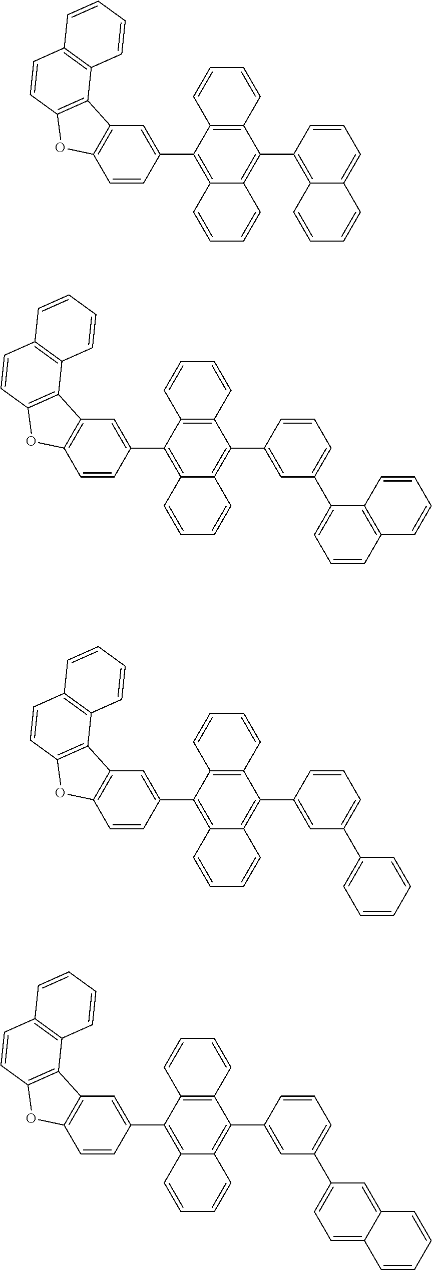 [SMILES: c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

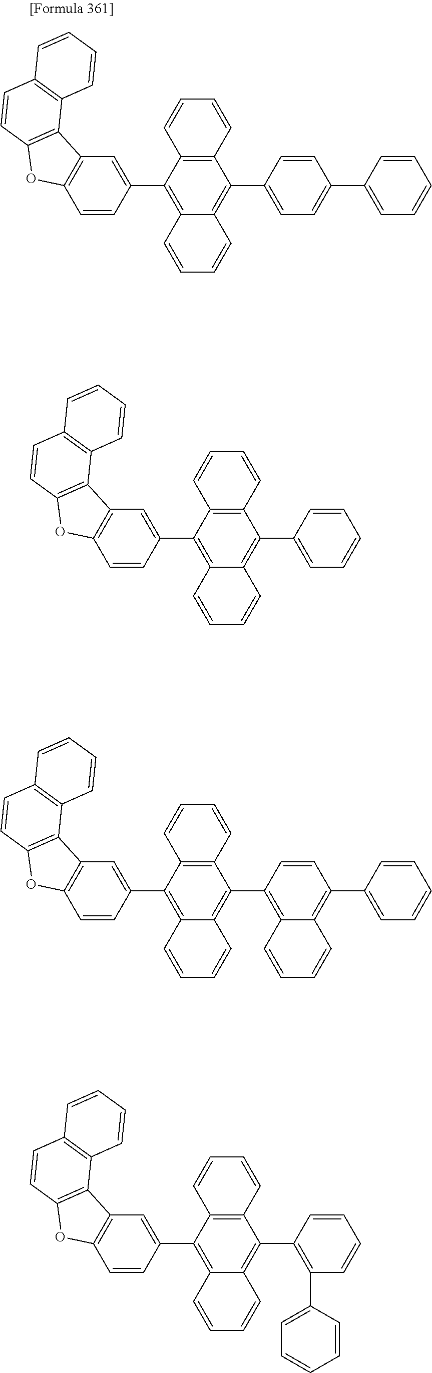 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1]

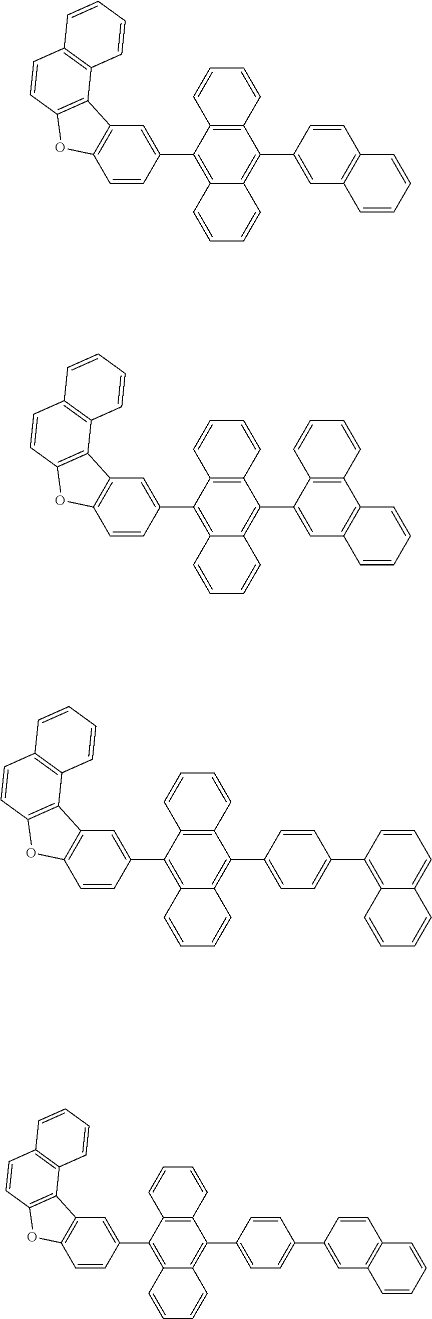 [SMILES: c1ccc2cc(-c3c4ccccc4c(-c4ccc5oc6ccc7ccccc7c6c5c4)c4ccccc34)ccc2c1 ; c1ccc2c(c1)cc(-c1c3ccccc3c(-c3ccc4oc5ccc6ccccc6c5c4c3)c3ccccc13)c1ccccc12 ; c1ccc2c(-c3ccc(-c4c5ccccc5c(-c5ccc6oc7ccc8ccccc8c7c6c5)c5ccccc45)cc3)cccc2c1 ; c1ccc2cc(-c3ccc(-c4c5ccccc5c(-c5ccc6oc7ccc8ccccc8c7c6c5)c5ccccc45)cc3)ccc2c1]

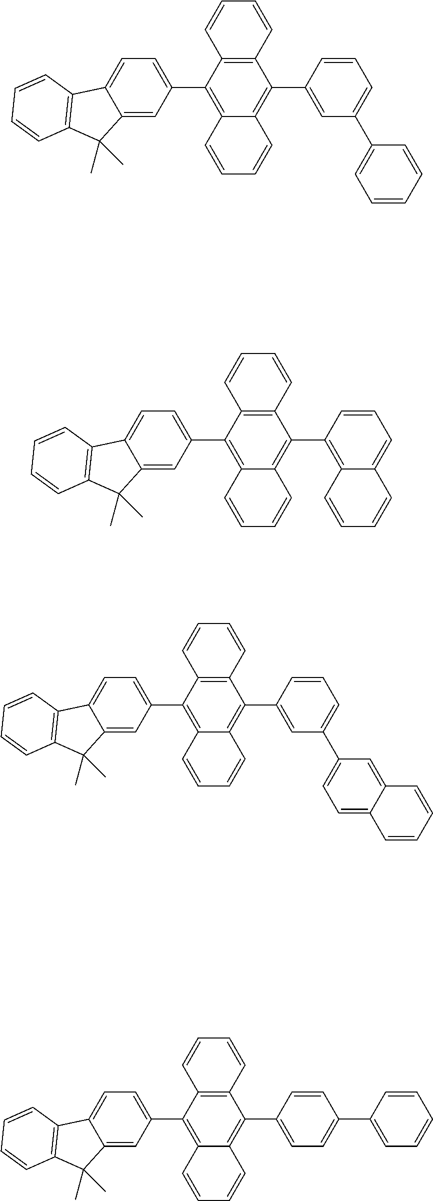

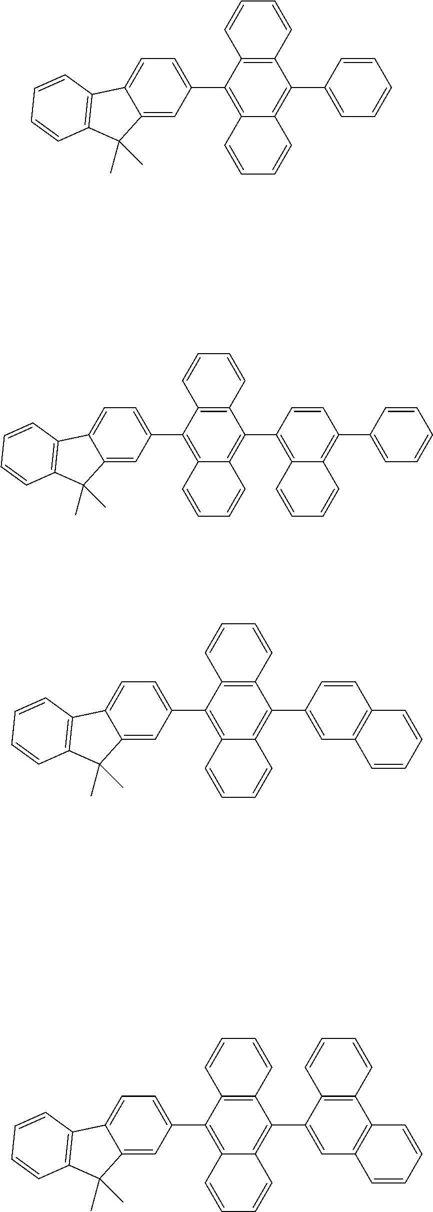

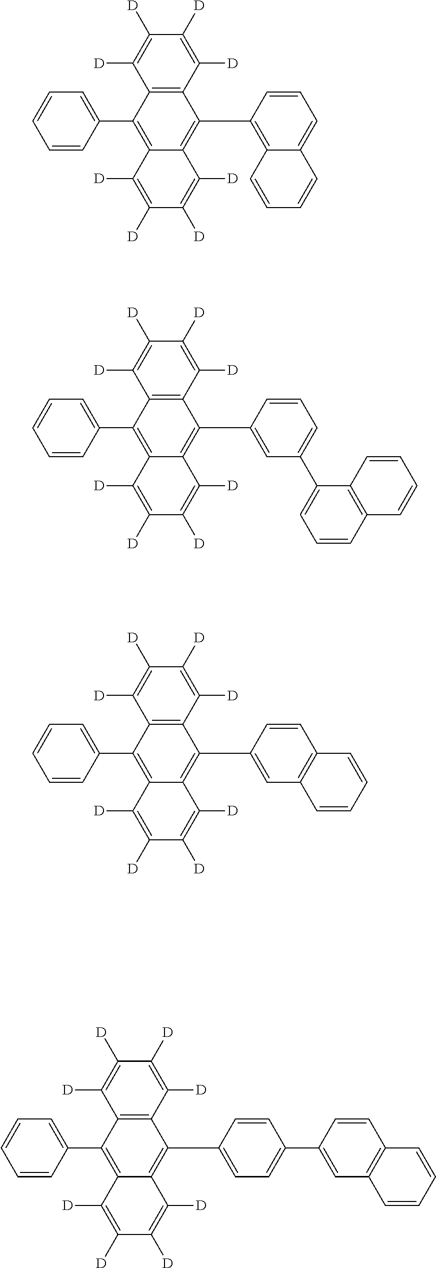 [SMILES: [2H]c1c([2H])c([2H])c2c(-c3cccc4ccccc34)c3c([2H])c([2H])c([2H])c([2H])c3c(-c3ccccc3)c2c1[2H] ; [2H]c1c([2H])c([2H])c2c(-c3cccc(-c4cccc5ccccc45)c3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3ccc(-c4ccc5ccccc5c4)cc3)c3c([2H])c([2H])c([2H])c([2H])c3c(-c3ccccc3)c2c1[2H]]

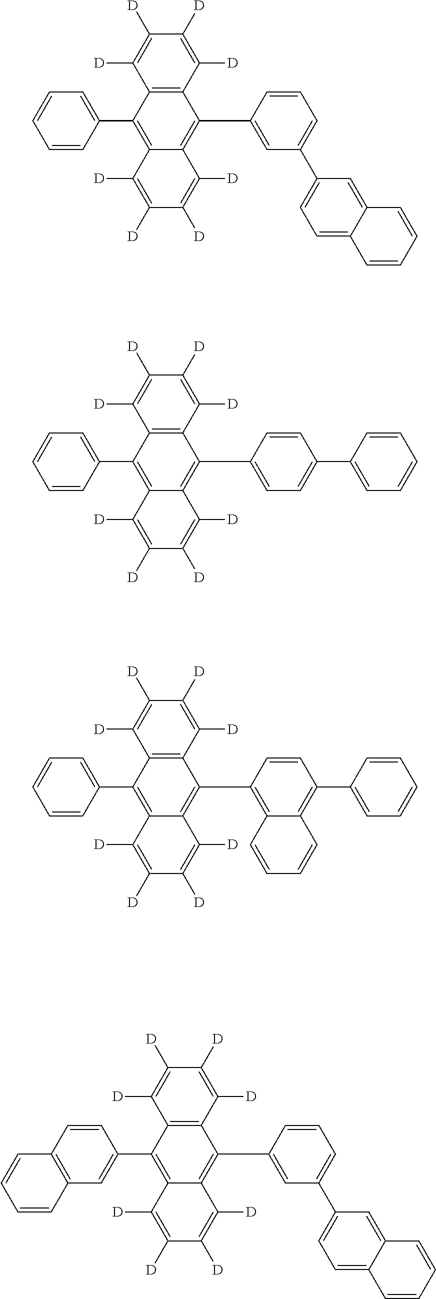 [SMILES: [2H]c1c([2H])c([2H])c2c(-c3cccc(-c4ccc5ccccc5c4)c3)c3c([2H])c([2H])c([2H])c([2H])c3c(-c3ccccc3)c2c1[2H] ; [2H]c1c([2H])c([2H])c2c(-c3ccc(-c4ccccc4)cc3)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3cccc(-c4ccc5ccccc5c4)c3)c2c1[2H]]

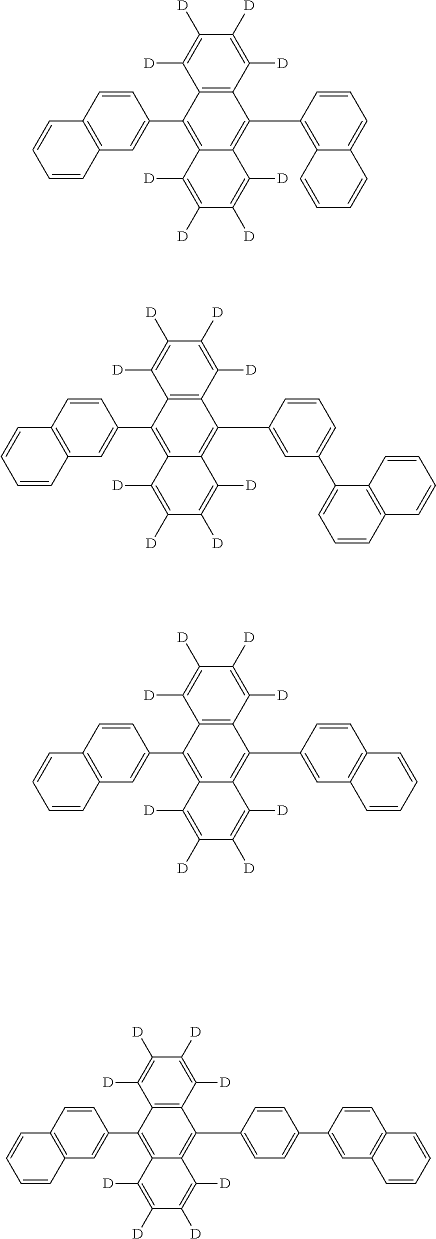 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4ccccc4c3)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccc(-c4ccc5ccccc5c4)cc3)c2c1[2H]]

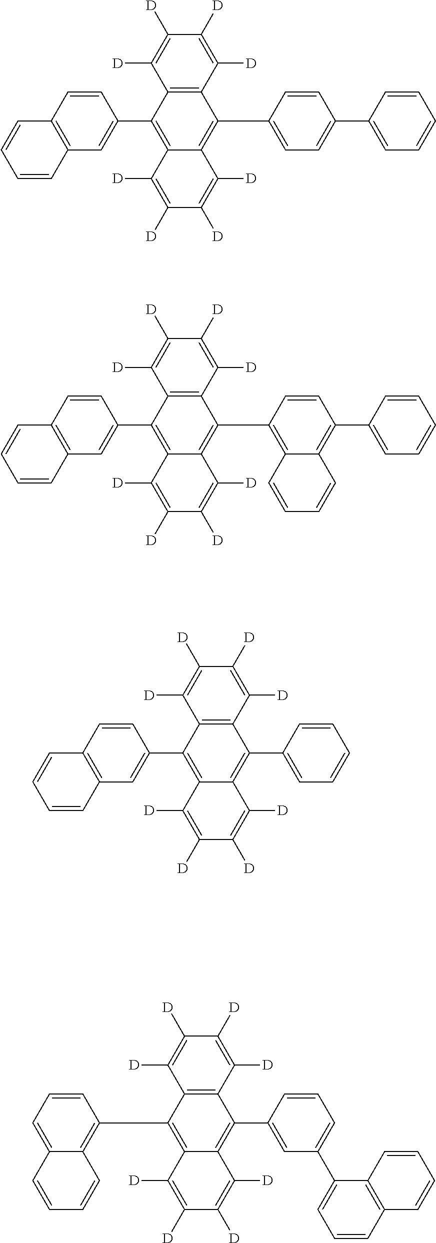 [SMILES: [2H]c1c([2H])c([2H])c2c(-c3ccc4ccccc4c3)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3cccc(-c4cccc5ccccc45)c3)c2c1[2H]]

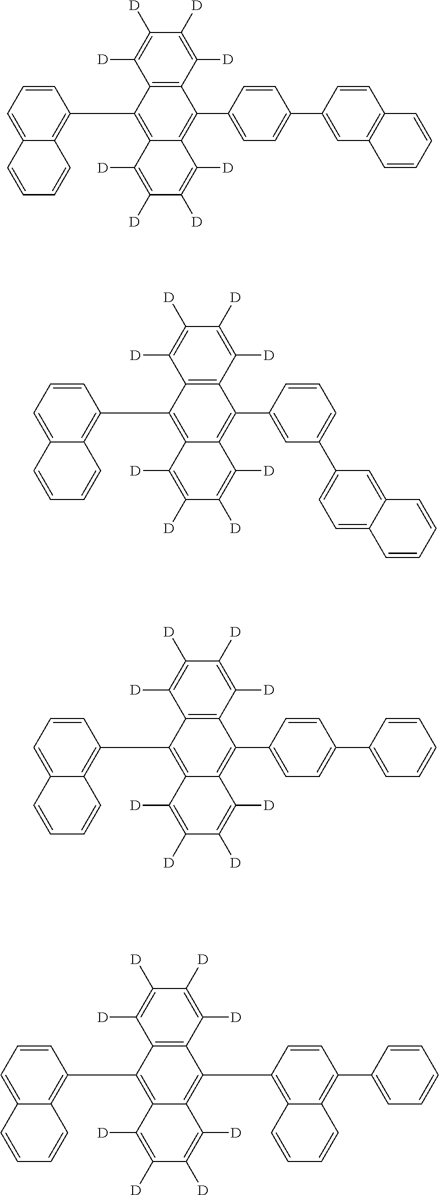 [SMILES: [2H]c1c([2H])c([2H])c2c(-c3cccc4ccccc34)c3c([2H])c([2H])c([2H])c([2H])c3c(-c3ccc(-c4ccc5ccccc5c4)cc3)c2c1[2H] ; [2H]c1c([2H])c([2H])c2c(-c3cccc4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3cccc4ccccc34)c2c1[2H]]

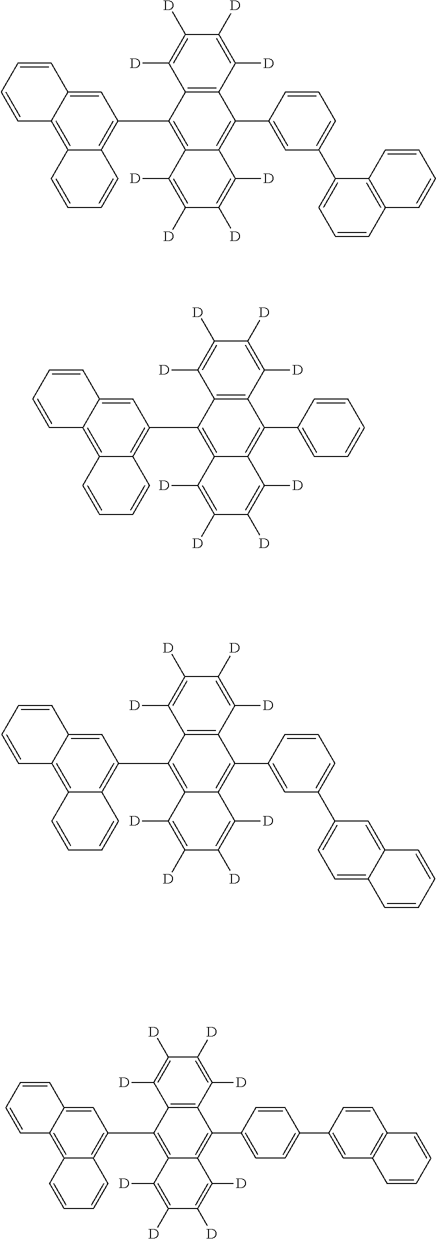 [SMILES: [2H]c1c([2H])c([2H])c2c(-c3cc4ccccc4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cc4ccccc4c4ccccc34)c3c([2H])c([2H])c([2H])c([2H])c3c(-c3ccccc3)c2c1[2H] ; [2H]c1c([2H])c([2H])c2c(-c3cc4ccccc4c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cc4ccccc4c4ccccc34)c3c([2H])c([2H])c([2H])c([2H])c3c(-c3ccc(-c4ccc5ccccc5c4)cc3)c2c1[2H]]

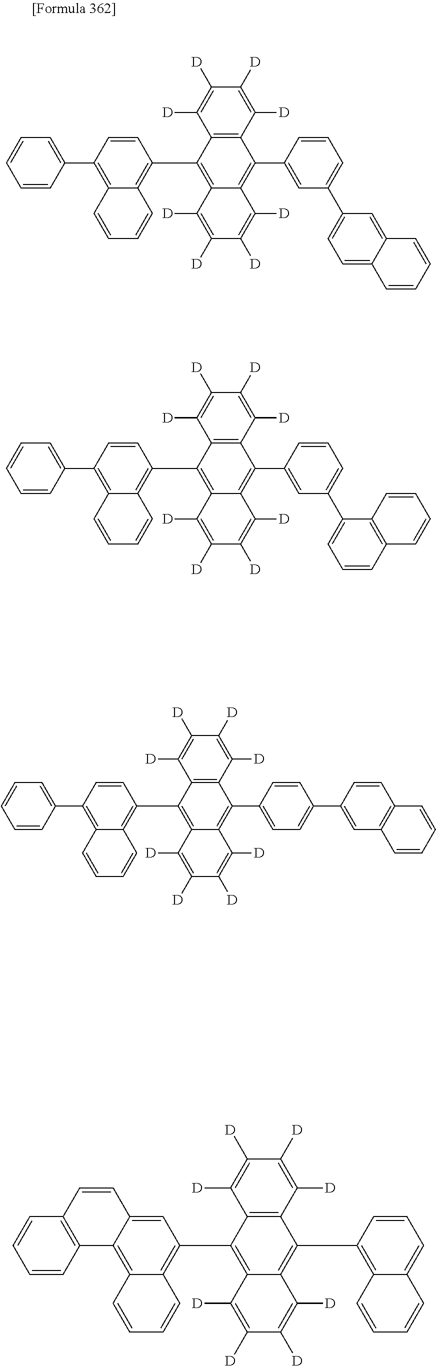 [SMILES: [2H]c1c([2H])c([2H])c2c(-c3ccc(-c4ccccc4)c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3ccc(-c4ccccc4)c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3ccc(-c4ccccc4)c4ccccc34)c3c([2H])c([2H])c([2H])c([2H])c3c(-c3ccc(-c4ccc5ccccc5c4)cc3)c2c1[2H] ; [2H]c1c([2H])c([2H])c2c(-c3cc4ccc5ccccc5c4c4ccccc34)c3c([2H])c([2H])c([2H])c([2H])c3c(-c3cccc4ccccc34)c2c1[2H]]

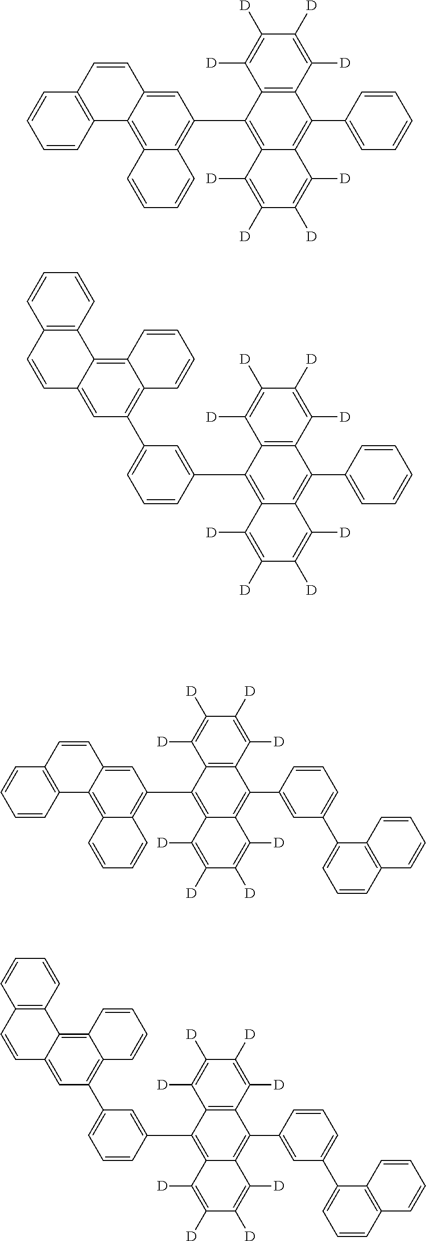 [SMILES: [2H]c1c([2H])c([2H])c2c(-c3cc4ccc5ccccc5c4c4ccccc34)c3c([2H])c([2H])c([2H])c([2H])c3c(-c3ccccc3)c2c1[2H] ; [2H]c1c([2H])c([2H])c2c(-c3cccc(-c4cc5ccc6ccccc6c5c5ccccc45)c3)c3c([2H])c([2H])c([2H])c([2H])c3c(-c3ccccc3)c2c1[2H] ; [2H]c1c([2H])c([2H])c2c(-c3cc4ccc5ccccc5c4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cccc(-c4cc5ccc6ccccc6c5c5ccccc45)c3)c3c([2H])c([2H])c([2H])c([2H])c3c(-c3cccc(-c4cccc5ccccc45)c3)c2c1[2H]]

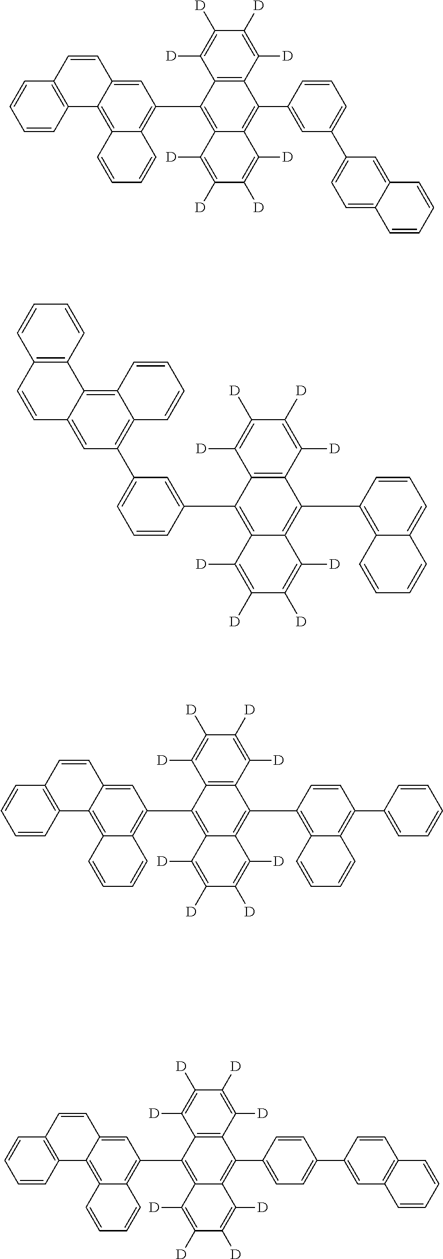 [SMILES: [2H]c1c([2H])c([2H])c2c(-c3cc4ccc5ccccc5c4c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3cccc(-c4cc5ccc6ccccc6c5c5ccccc45)c3)c2c1[2H] ; [2H]c1c([2H])c([2H])c2c(-c3cc4ccc5ccccc5c4c4ccccc34)c3c([2H])c([2H])c([2H])c([2H])c3c(-c3ccc(-c4ccccc4)c4ccccc34)c2c1[2H] ; [2H]c1c([2H])c([2H])c2c(-c3cc4ccc5ccccc5c4c4ccccc34)c3c([2H])c([2H])c([2H])c([2H])c3c(-c3ccc(-c4ccc5ccccc5c4)cc3)c2c1[2H]]

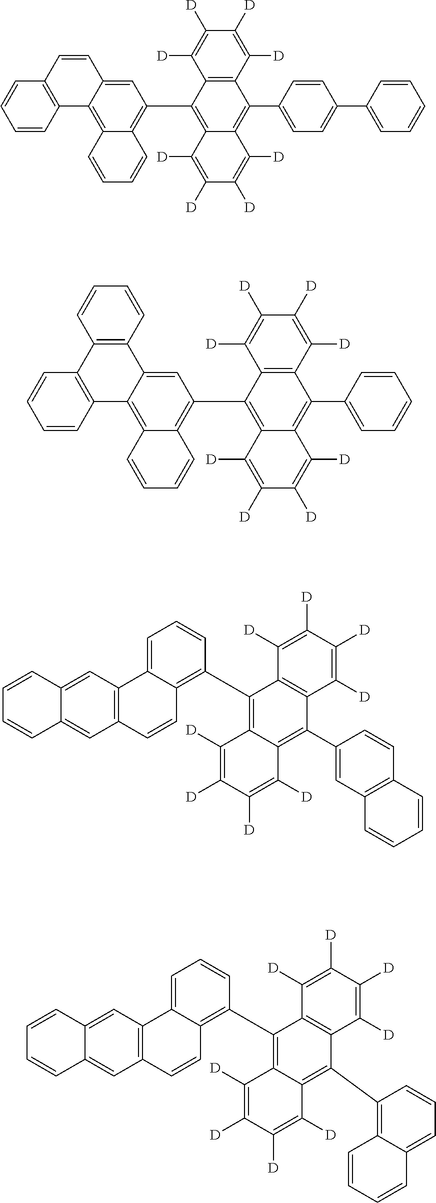 [SMILES: [2H]c1c([2H])c([2H])c2c(-c3cc4ccc5ccccc5c4c4ccccc34)c3c([2H])c([2H])c([2H])c([2H])c3c(-c3ccc(-c4ccccc4)cc3)c2c1[2H] ; [2H]c1c([2H])c([2H])c2c(-c3cc4c5ccccc5c5ccccc5c4c4ccccc34)c3c([2H])c([2H])c([2H])c([2H])c3c(-c3ccccc3)c2c1[2H] ; [2H]c1c([2H])c([2H])c2c(-c3cccc4c3ccc3cc5ccccc5cc34)c3c([2H])c([2H])c([2H])c([2H])c3c(-c3ccc4ccccc4c3)c2c1[2H] ; [2H]c1c([2H])c([2H])c2c(-c3cccc4c3ccc3cc5ccccc5cc34)c3c([2H])c([2H])c([2H])c([2H])c3c(-c3cccc4ccccc34)c2c1[2H]]

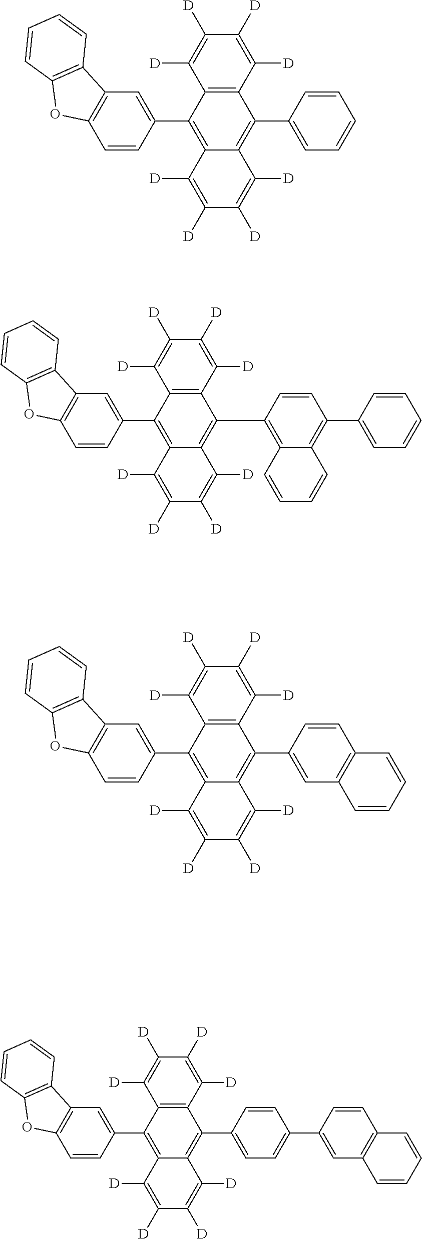 [SMILES: [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc4ccccc4c3)c2c1[2H] ; [2H]c1c([2H])c([2H])c2c(-c3ccc4oc5ccccc5c4c3)c3c([2H])c([2H])c([2H])c([2H])c3c(-c3ccc(-c4ccc5ccccc5c4)cc3)c2c1[2H]]

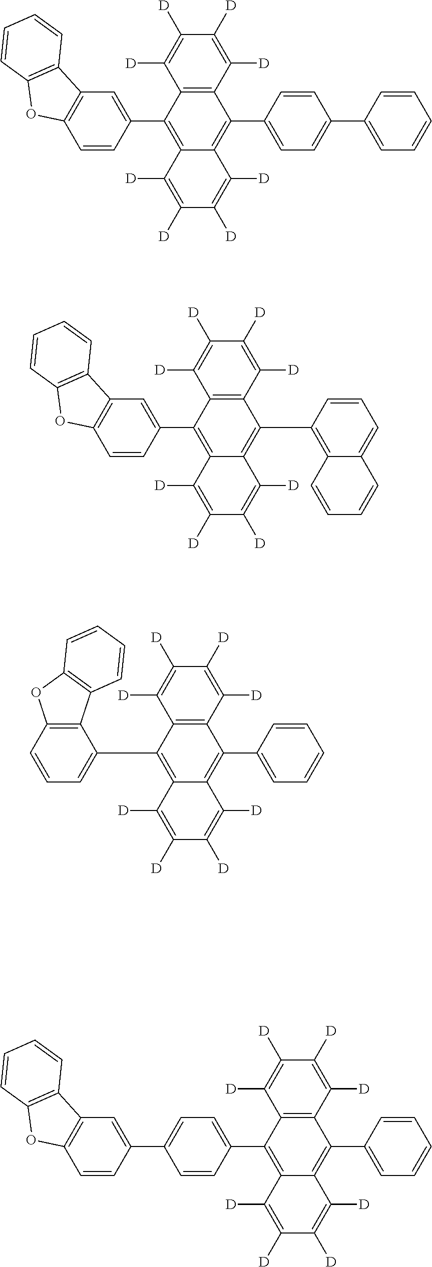 [SMILES: [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccccc5c4c3)c2c1[2H] ; [2H]c1c([2H])c([2H])c2c(-c3cccc4oc5ccccc5c34)c3c([2H])c([2H])c([2H])c([2H])c3c(-c3ccccc3)c2c1[2H] ; [2H]c1c([2H])c([2H])c2c(-c3ccc(-c4ccc5oc6ccccc6c5c4)cc3)c3c([2H])c([2H])c([2H])c([2H])c3c(-c3ccccc3)c2c1[2H]]

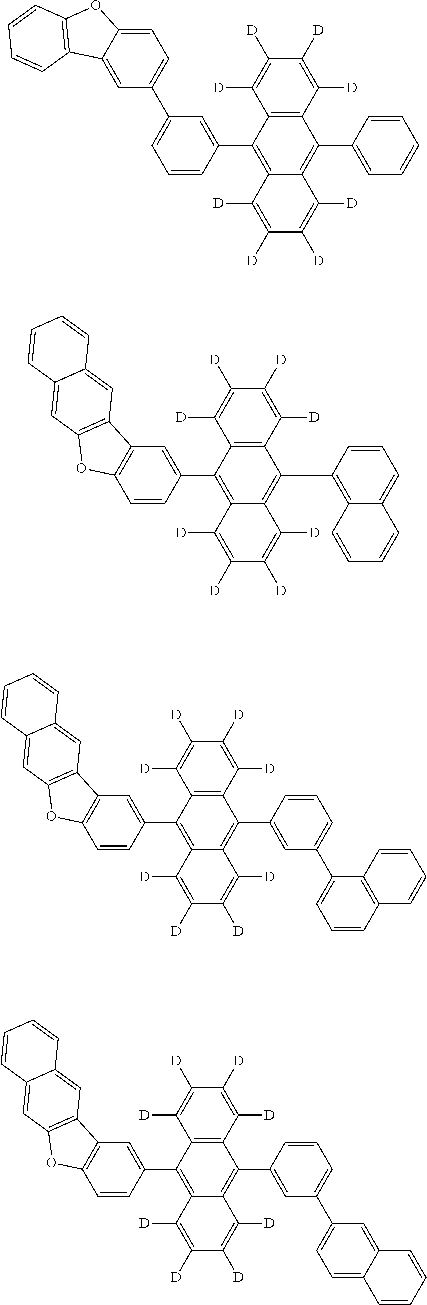 [SMILES: [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4ccc5ccccc5c4)c3)c2c1[2H]]

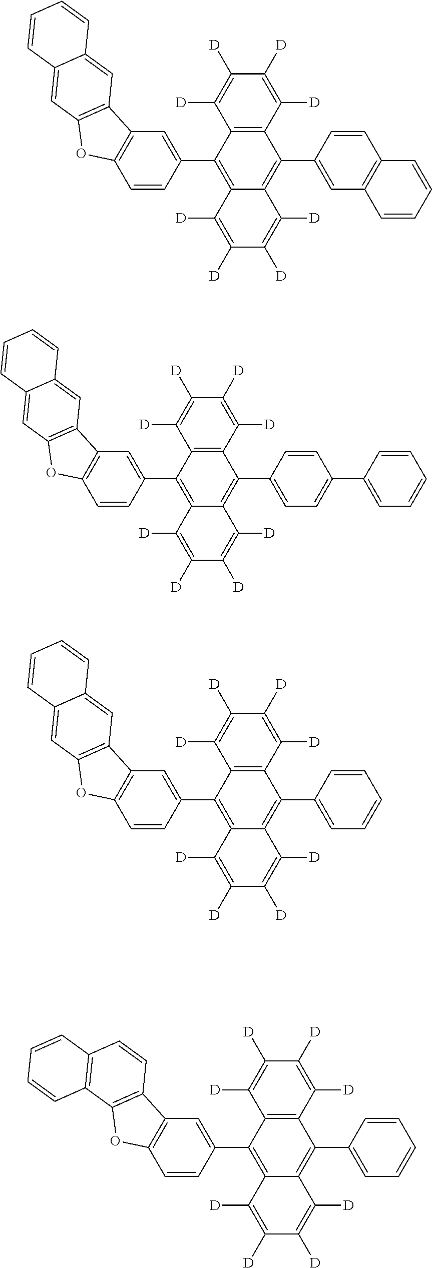 [SMILES: [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H] ; [2H]c1c([2H])c([2H])c2c(-c3ccc4oc5c6ccccc6ccc5c4c3)c3c([2H])c([2H])c([2H])c([2H])c3c(-c3ccccc3)c2c1[2H]]

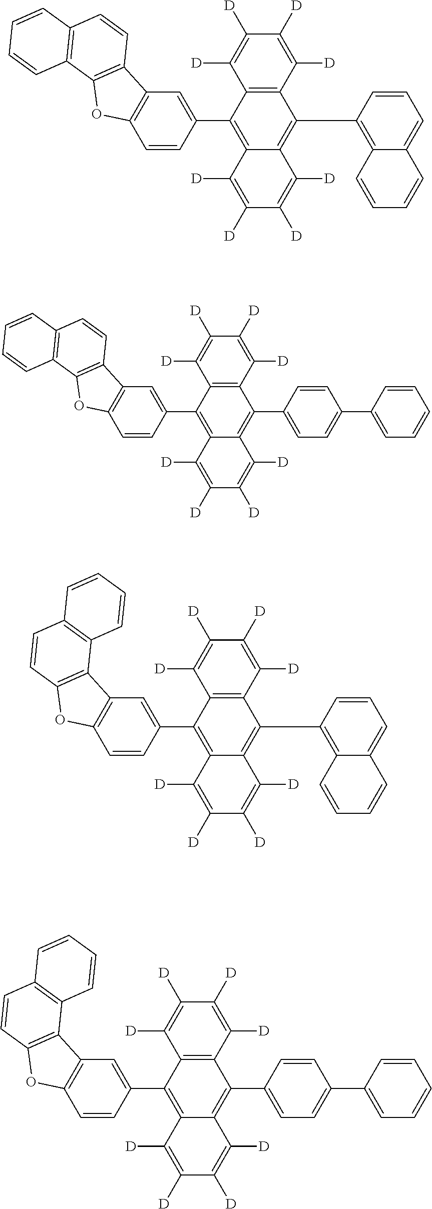 [SMILES: [2H]c1c([2H])c([2H])c2c(-c3cccc4ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccc6ccccc6c5c4c3)c2c1[2H] ; [2H]c1c([2H])c([2H])c2c(-c3ccc4oc5ccc6ccccc6c5c4c3)c3c([2H])c([2H])c([2H])c([2H])c3c(-c3ccc(-c4ccccc4)cc3)c2c1[2H]]

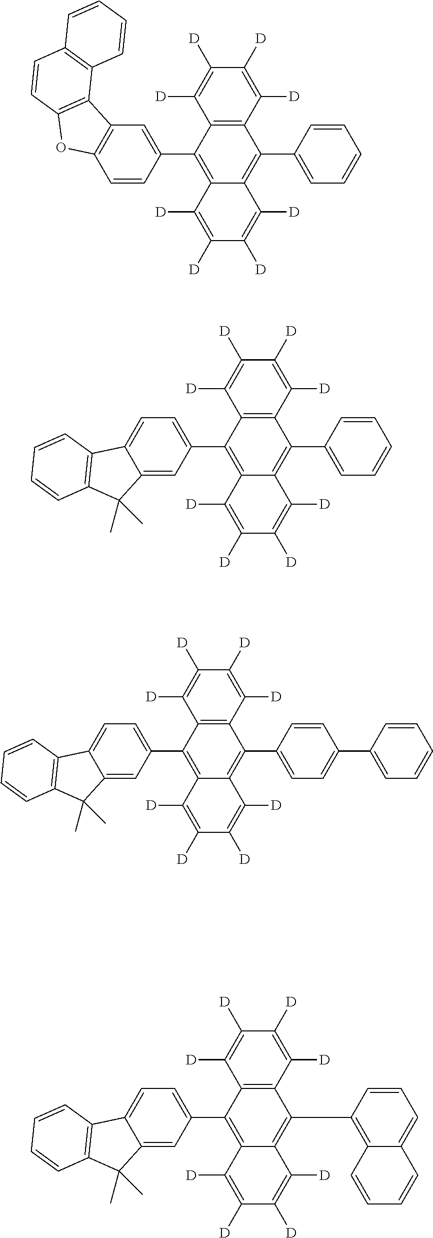 [SMILES: [2H]c1c([2H])c([2H])c2c(-c3ccc4oc5ccc6ccccc6c5c4c3)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4c(c3)C(C)(C)c3ccccc3-4)c2c1[2H]]

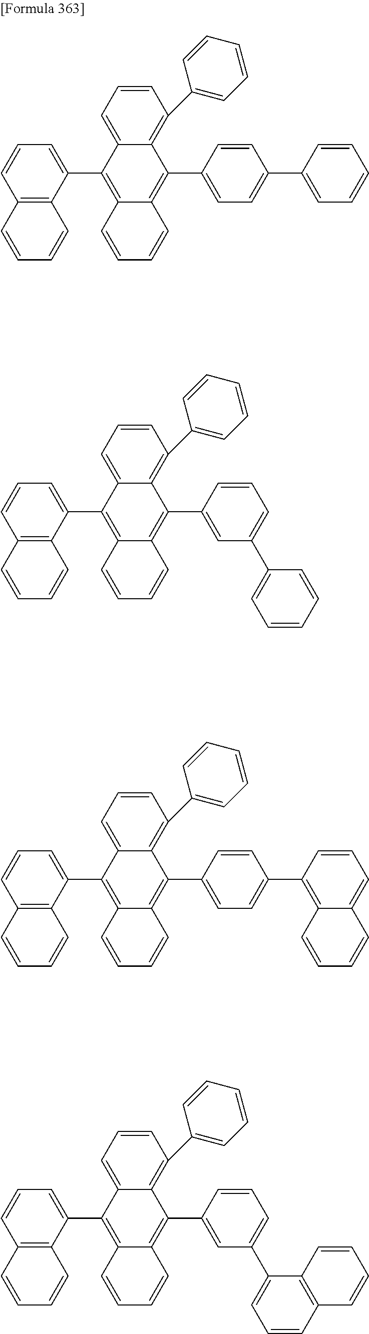

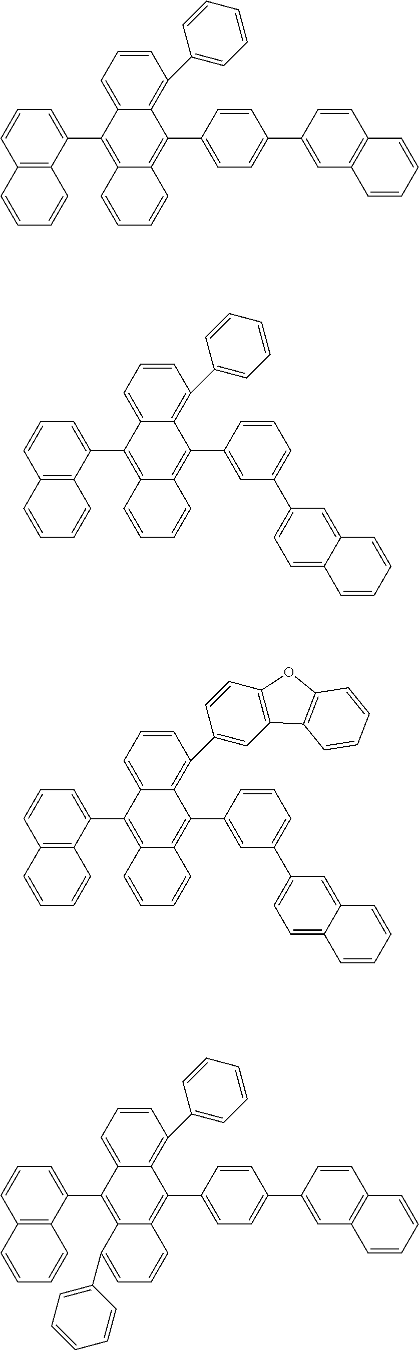

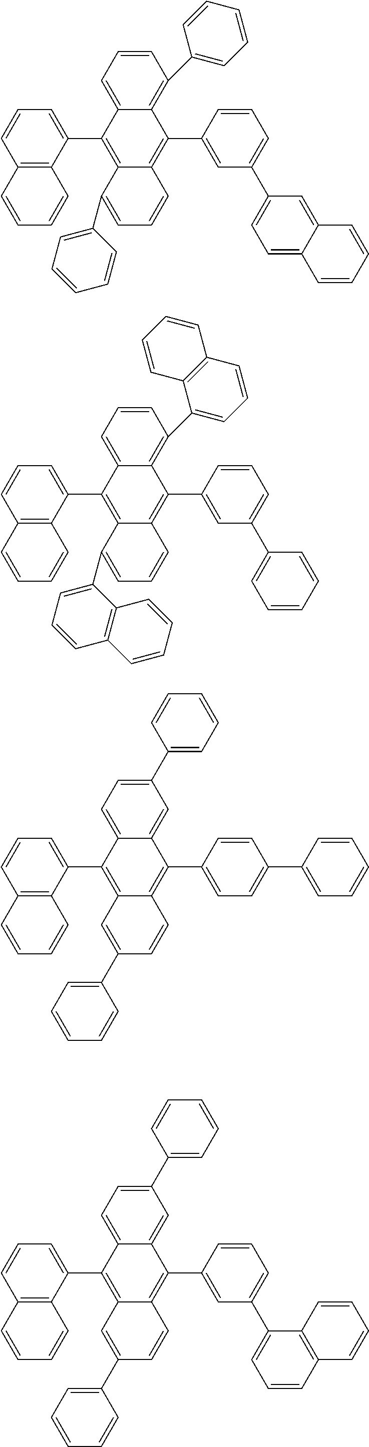

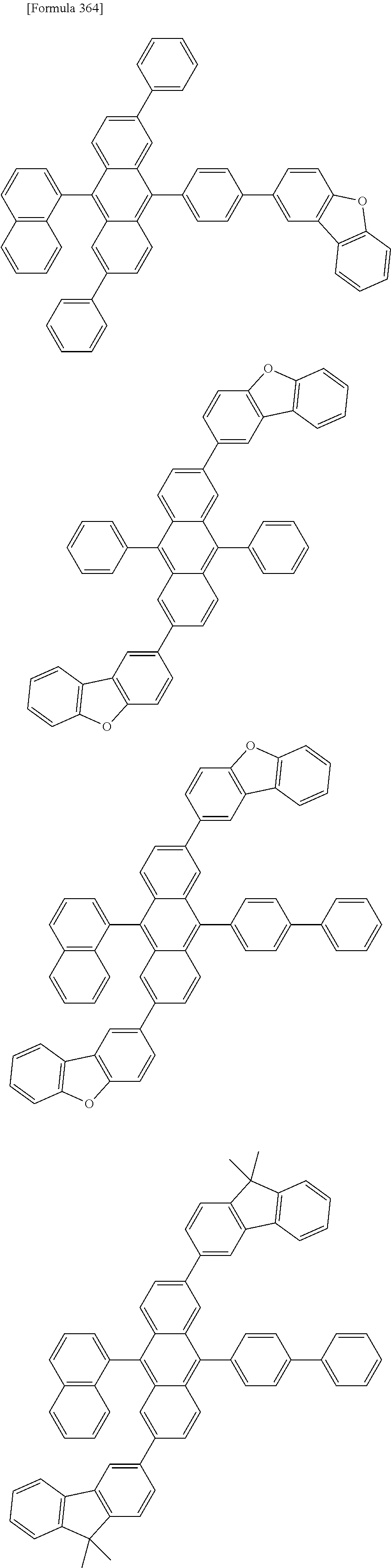

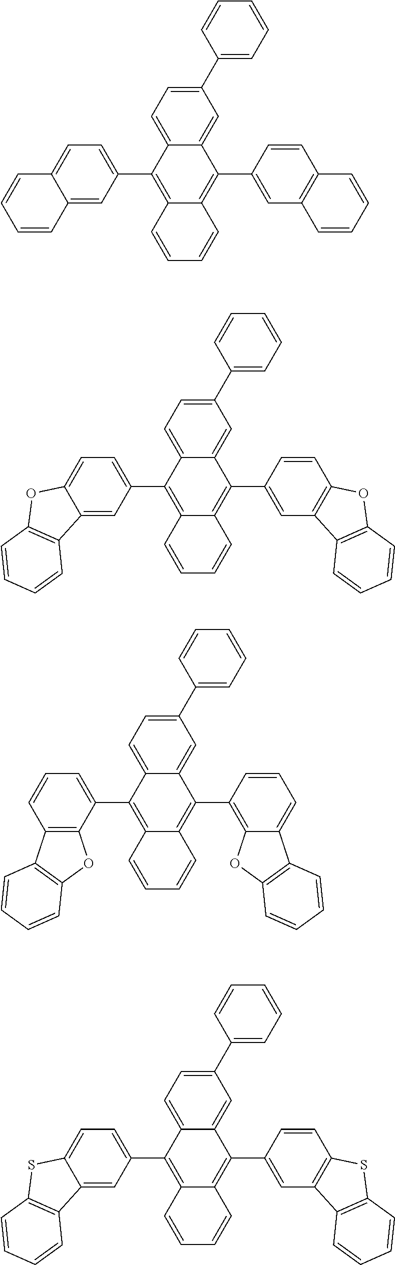

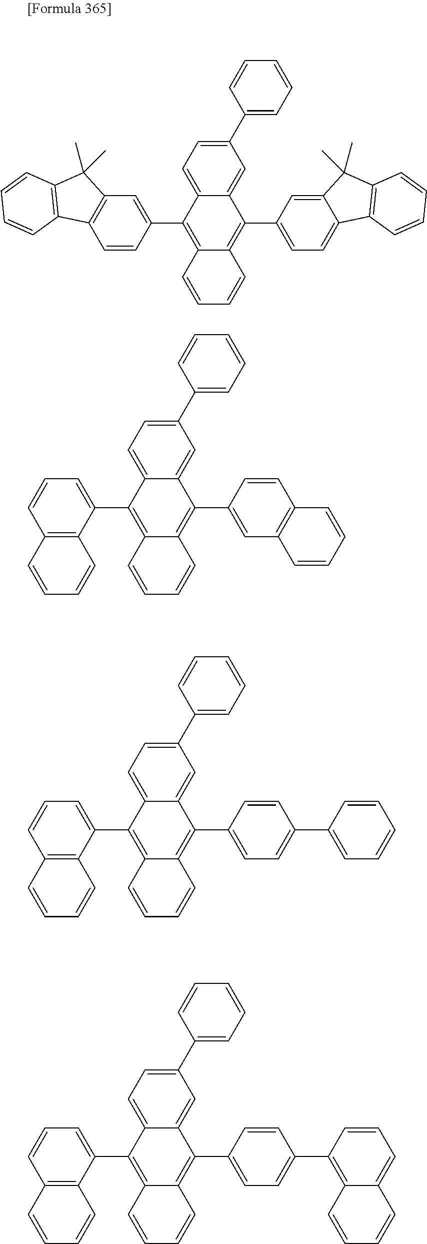

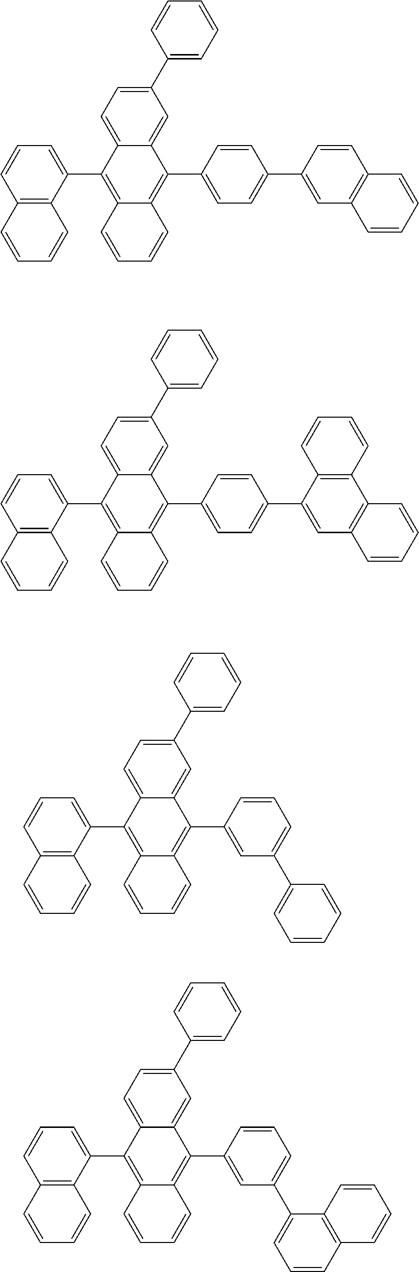

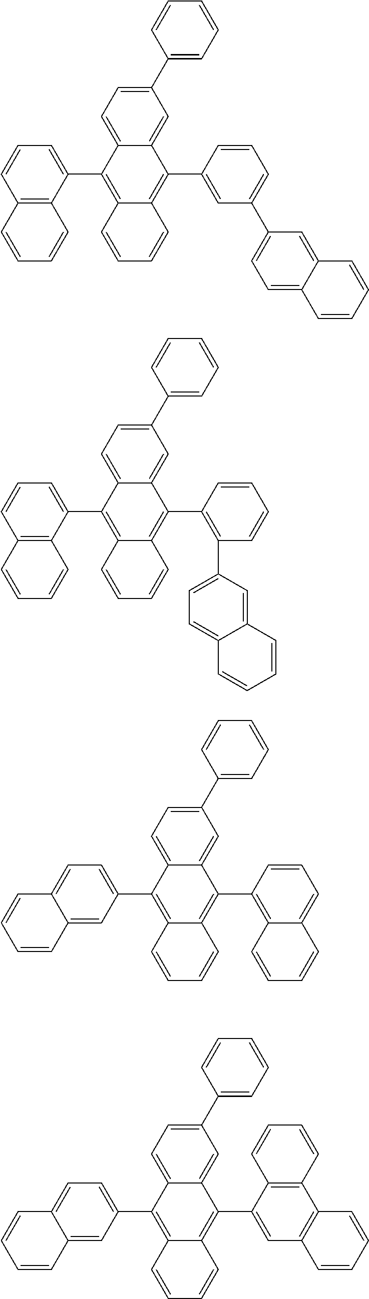

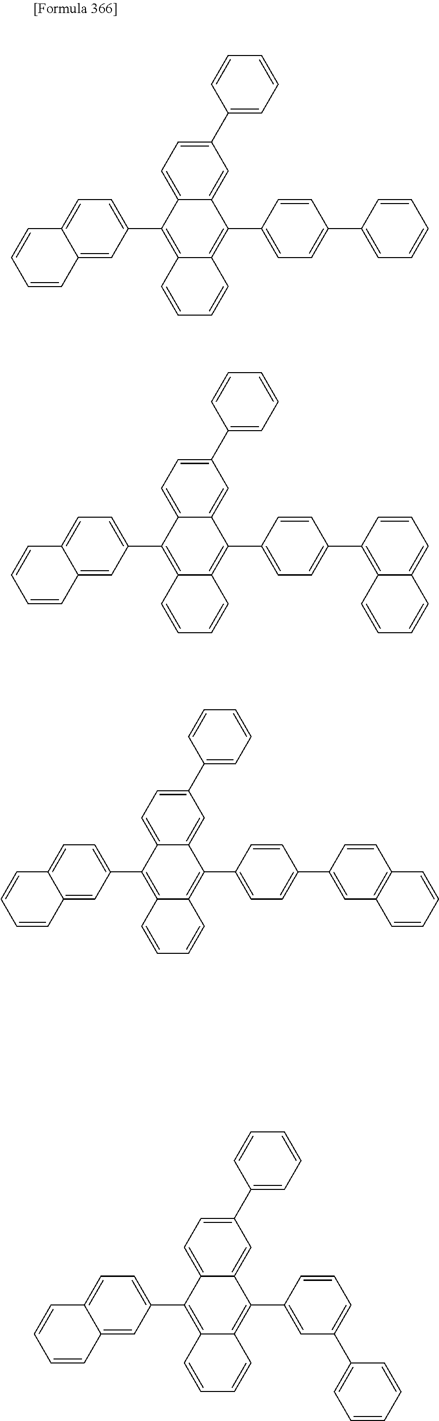

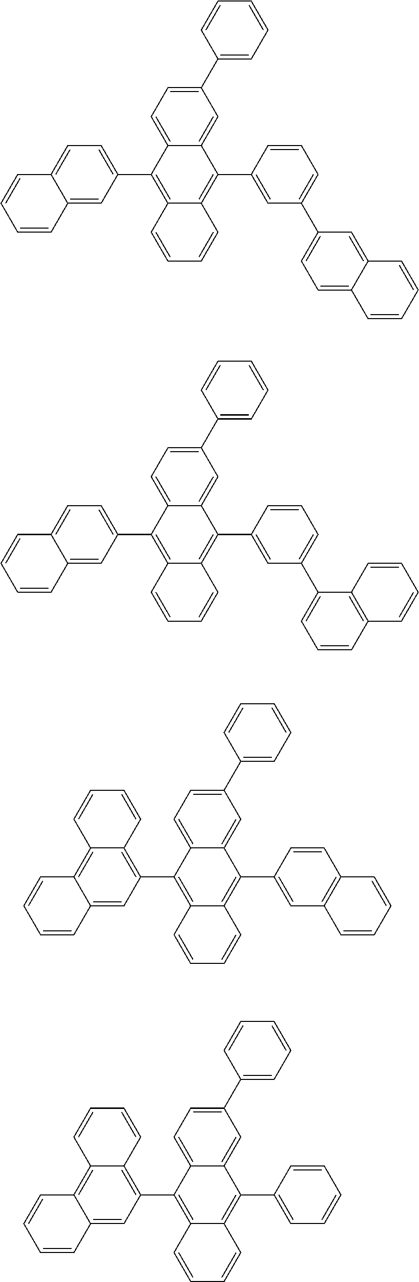

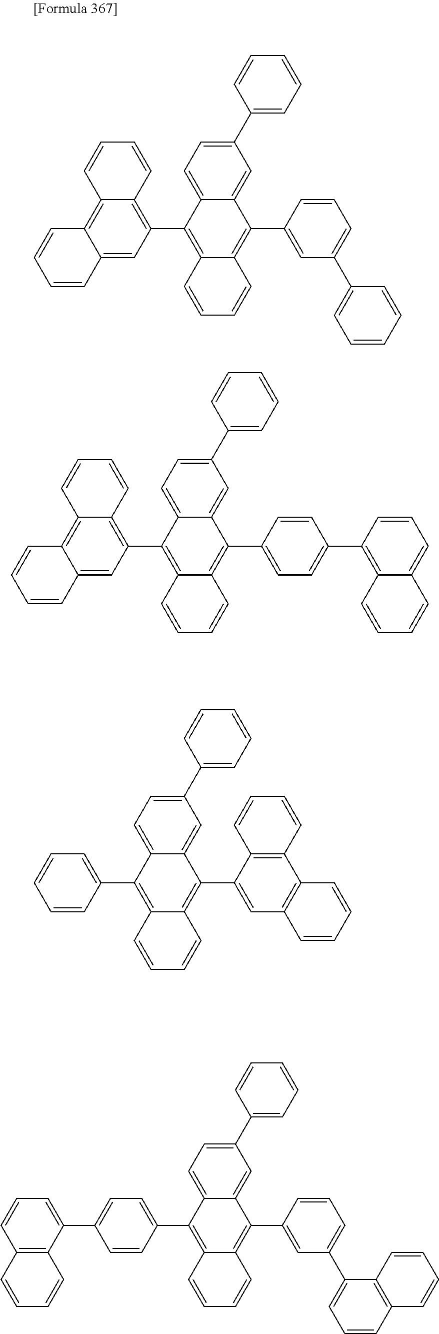

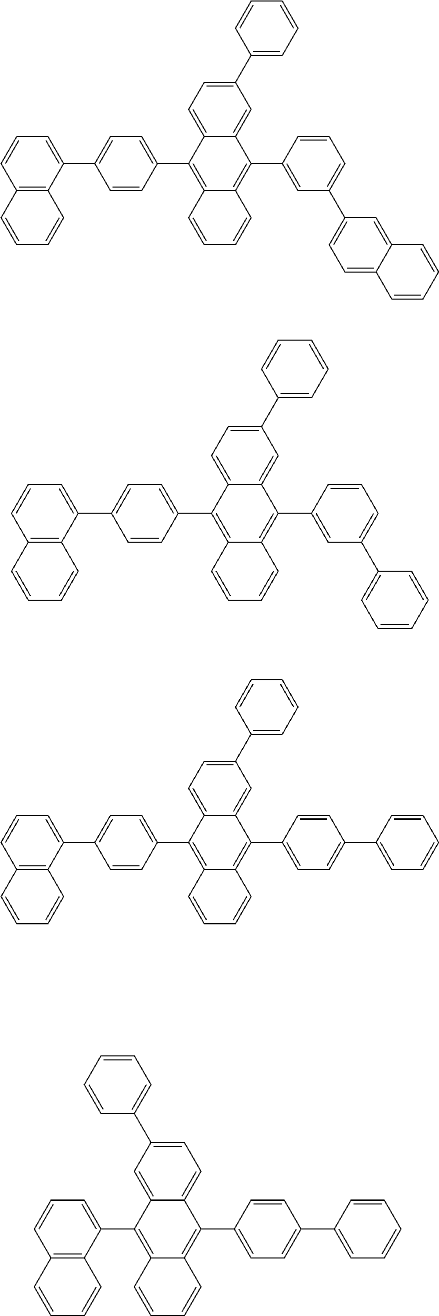

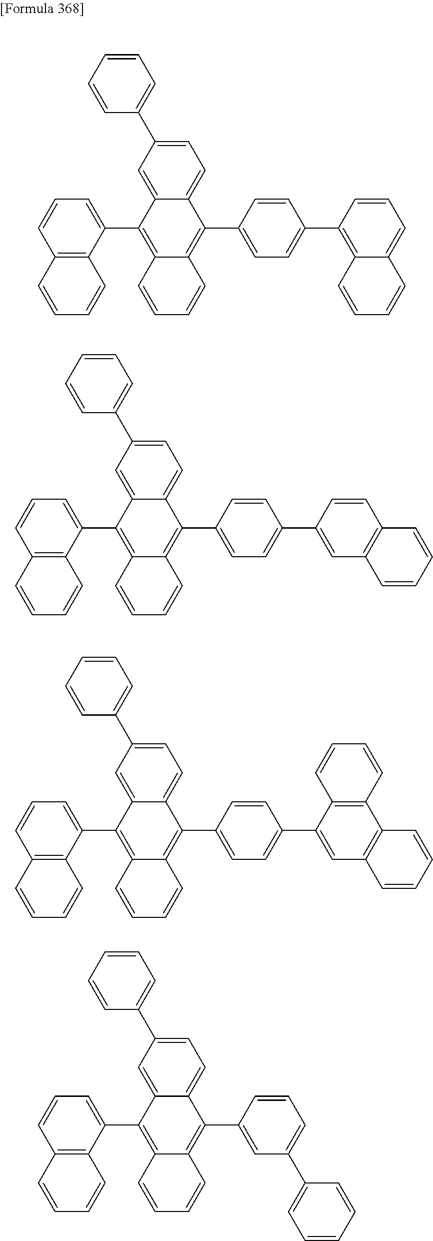

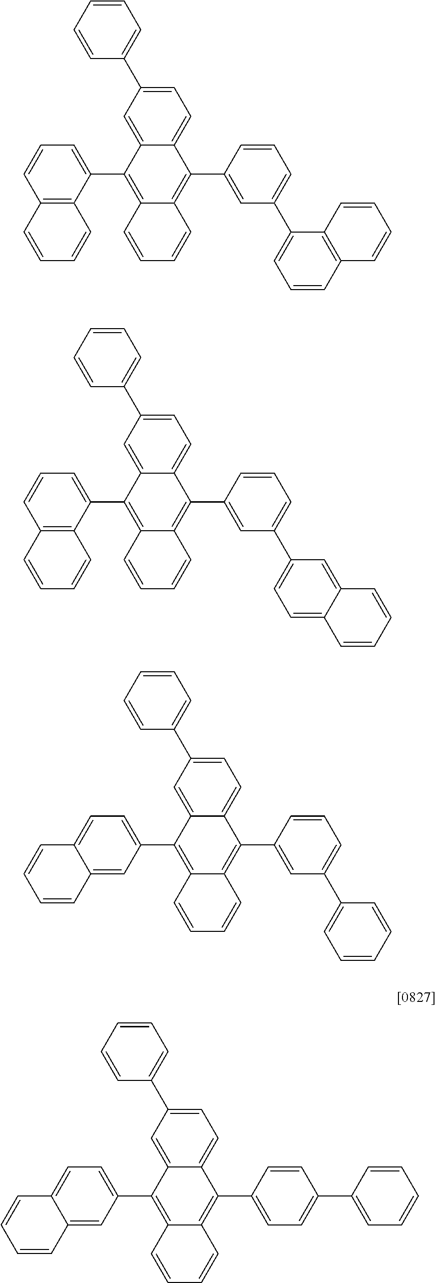

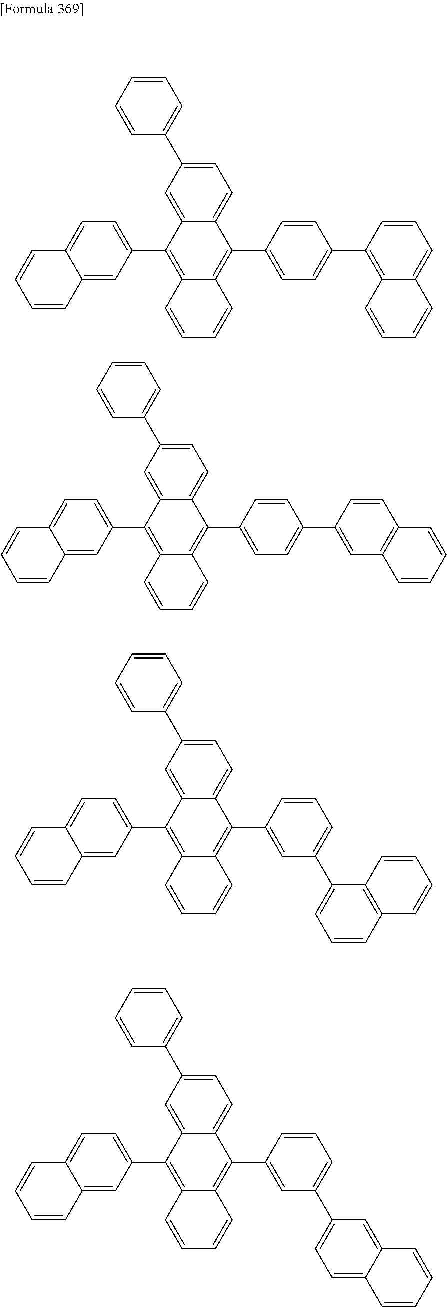

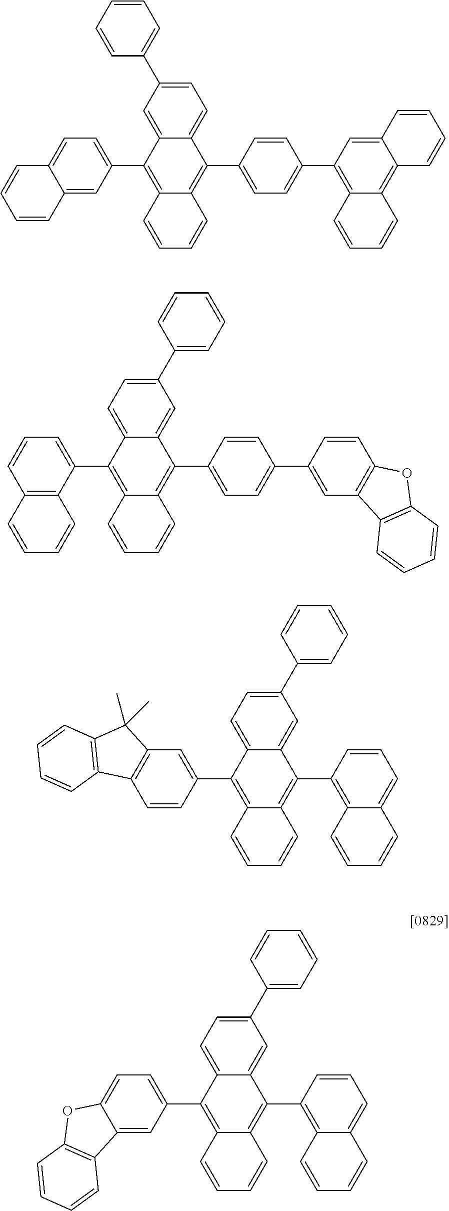

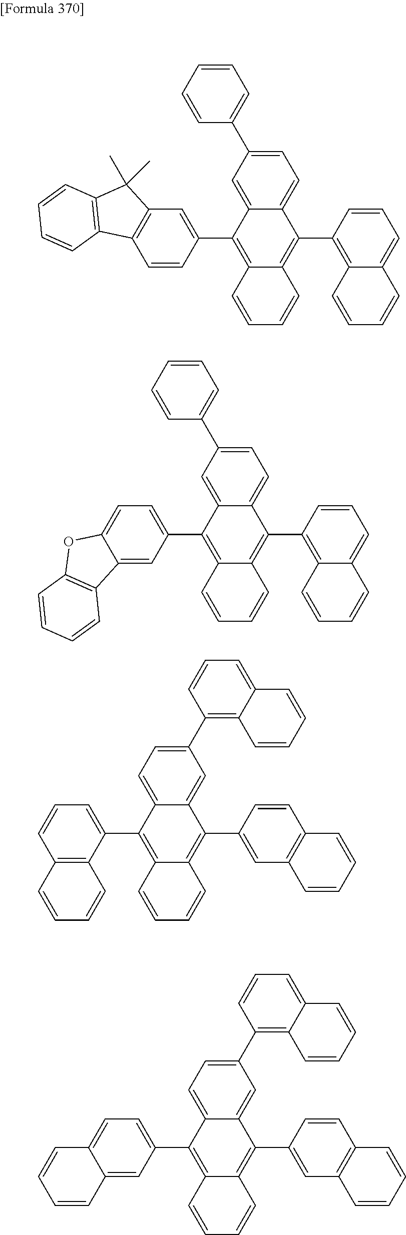

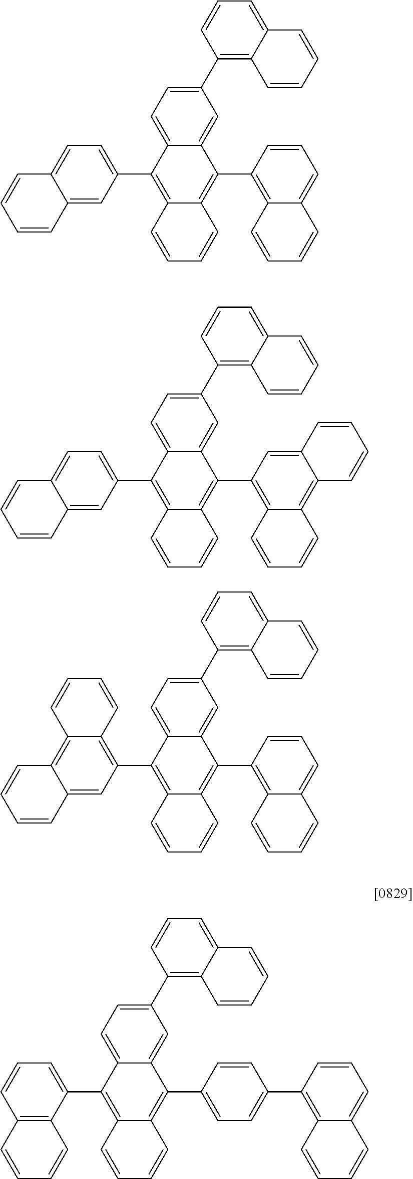

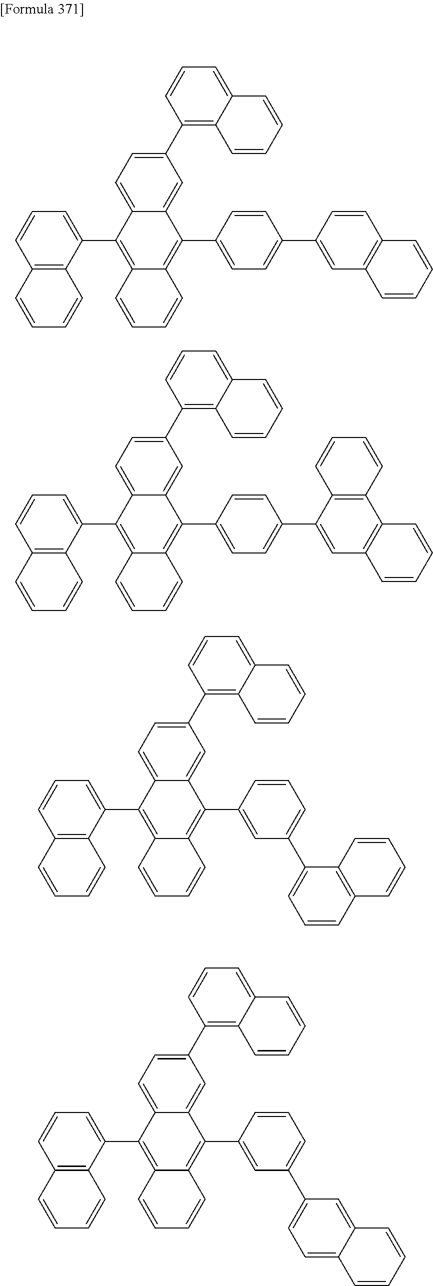

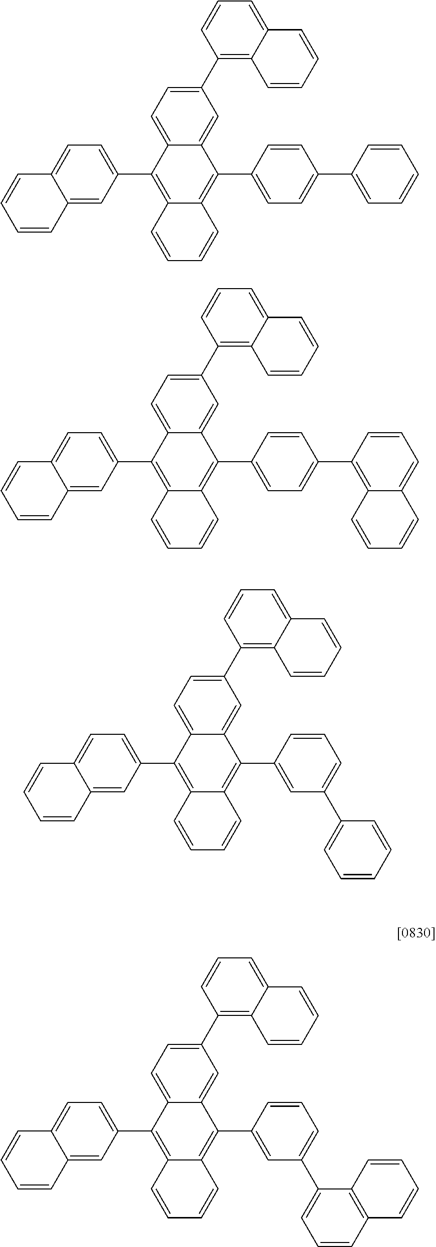

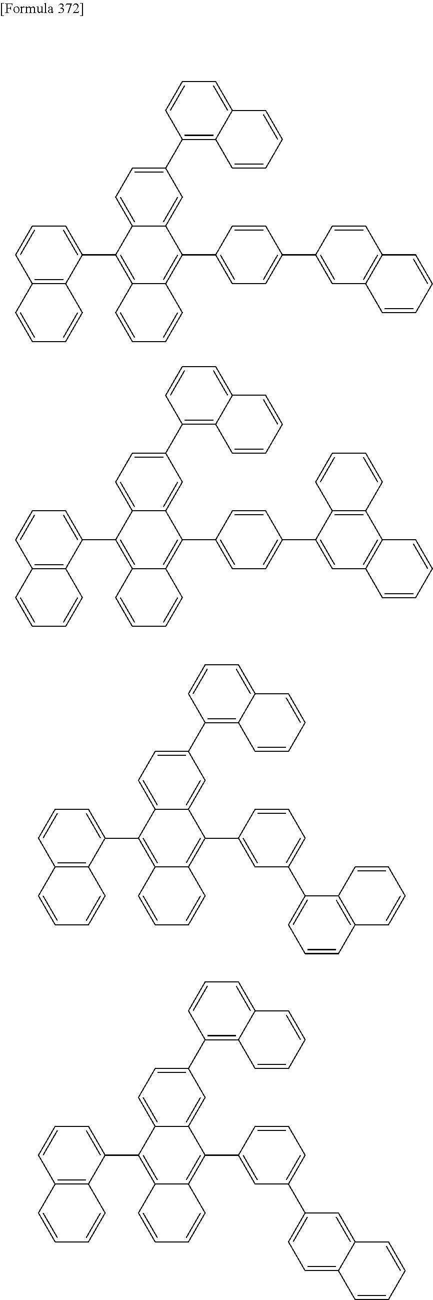

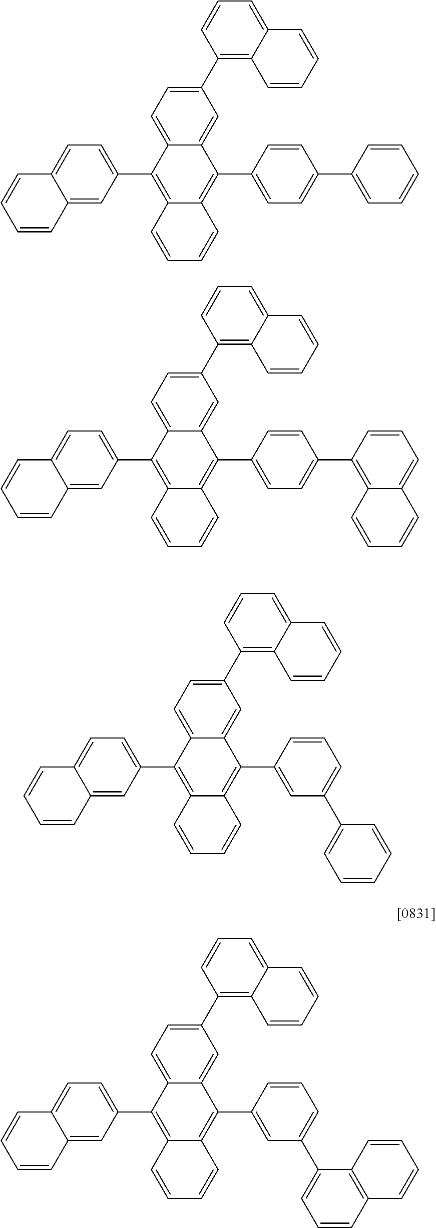

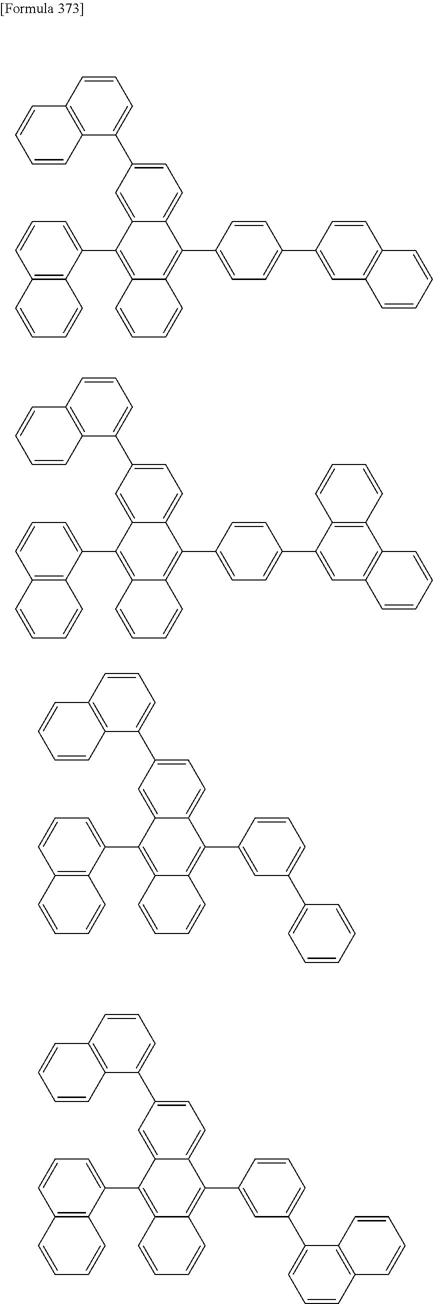

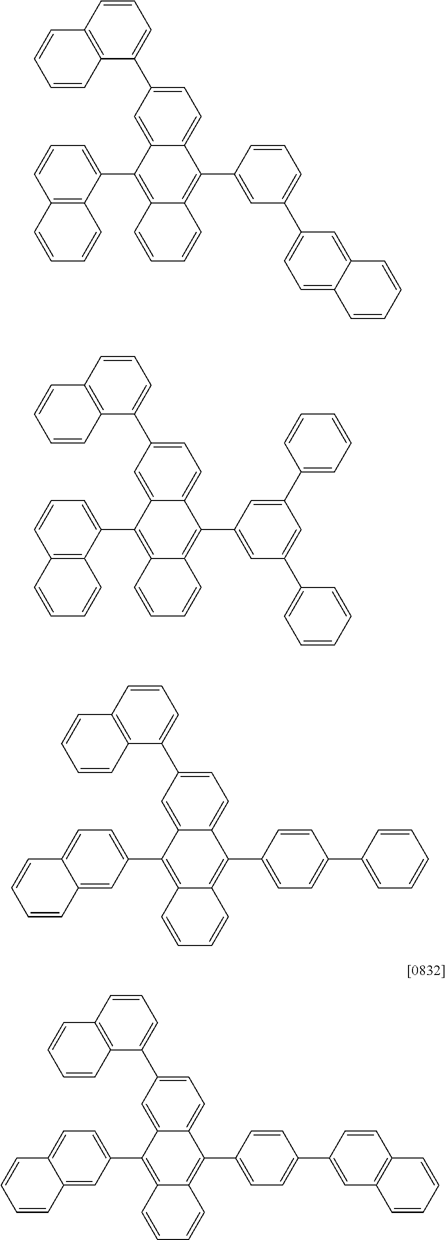

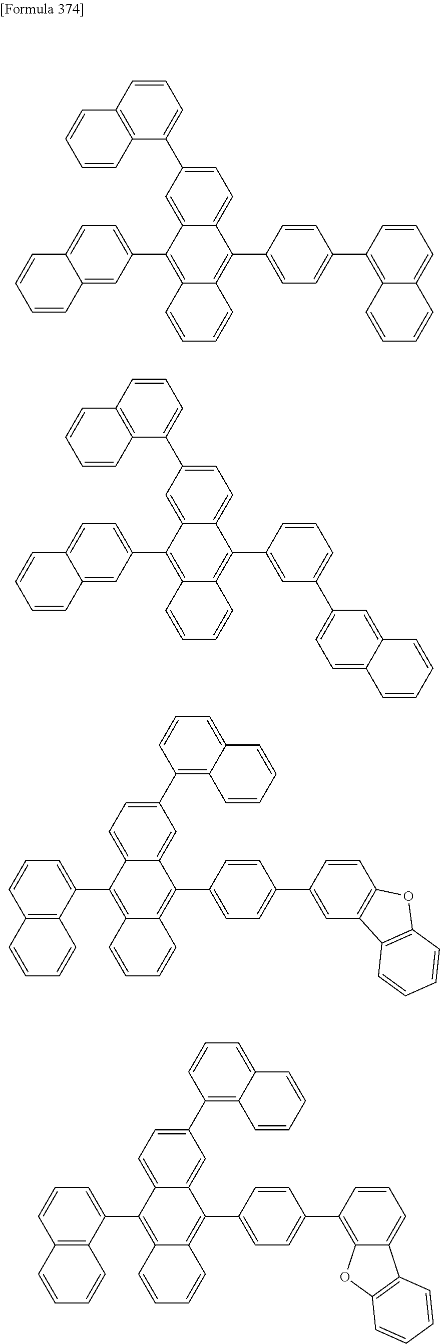

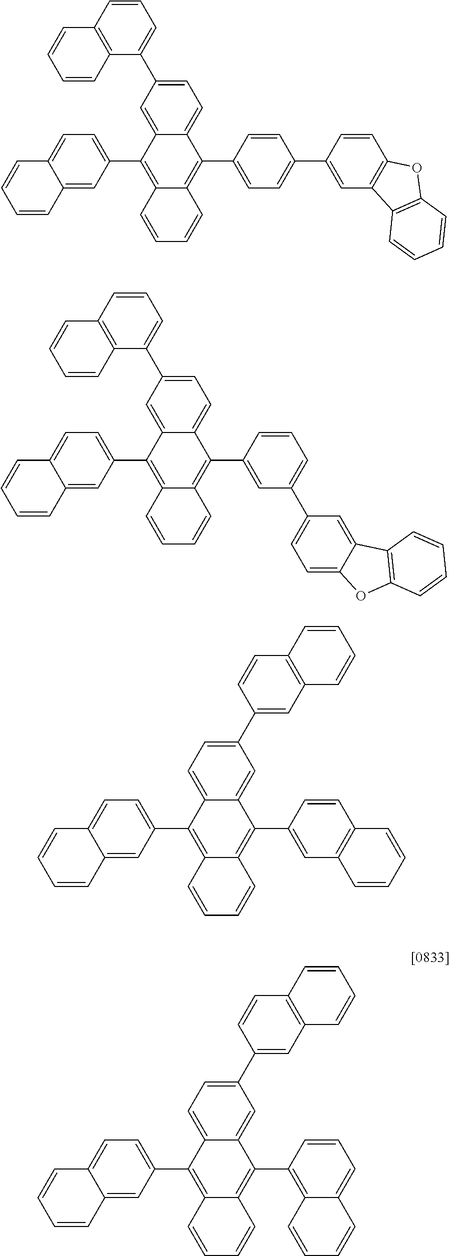

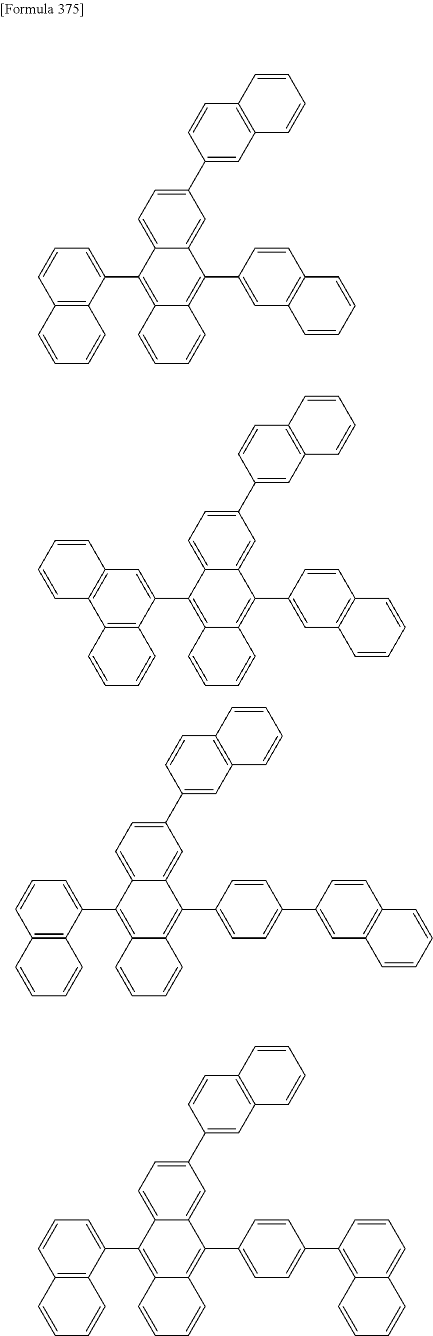

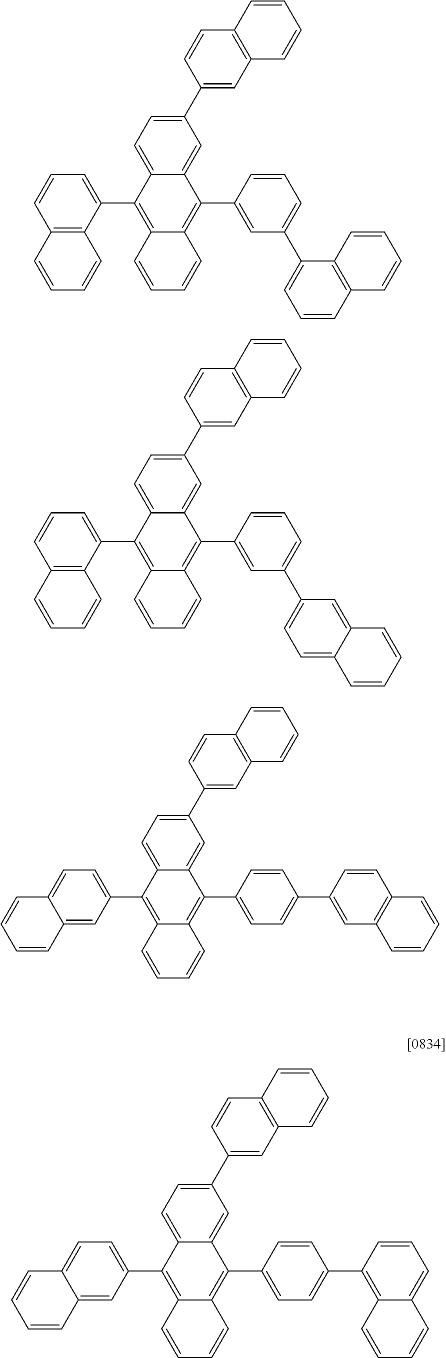

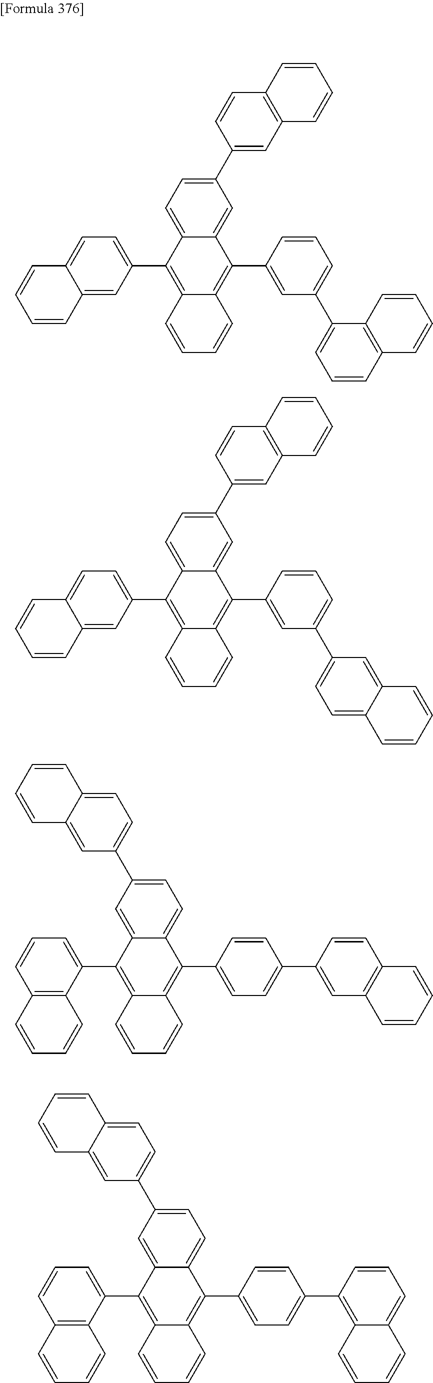

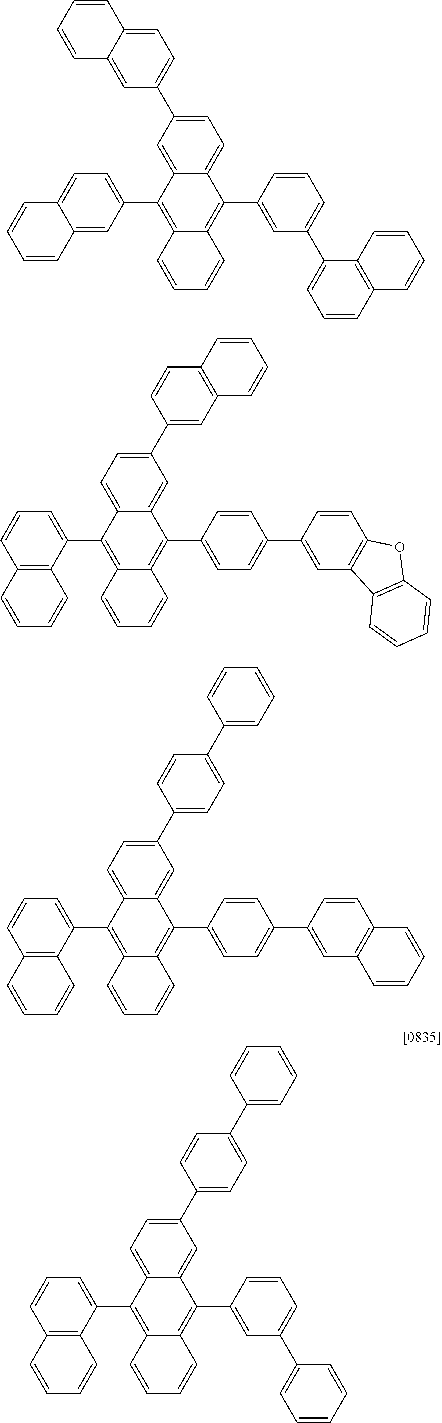

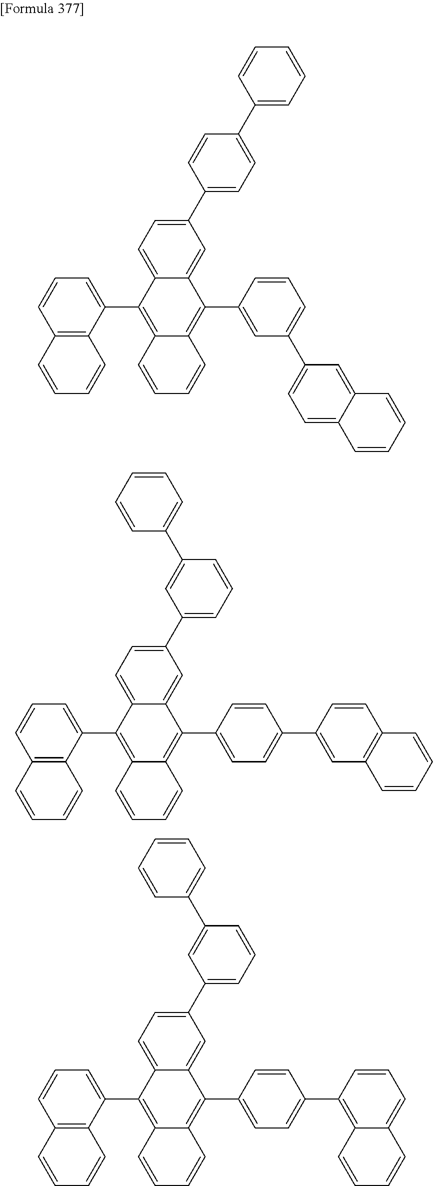

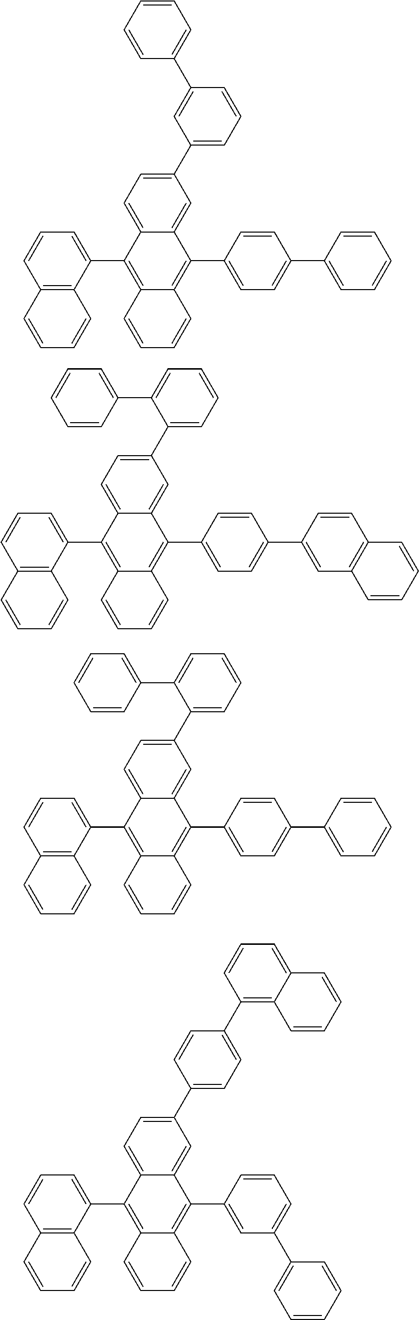

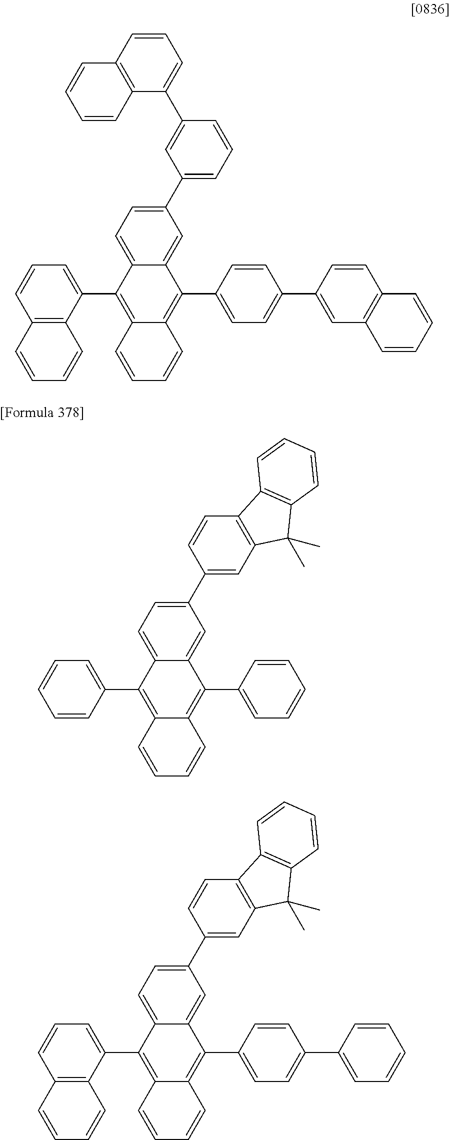

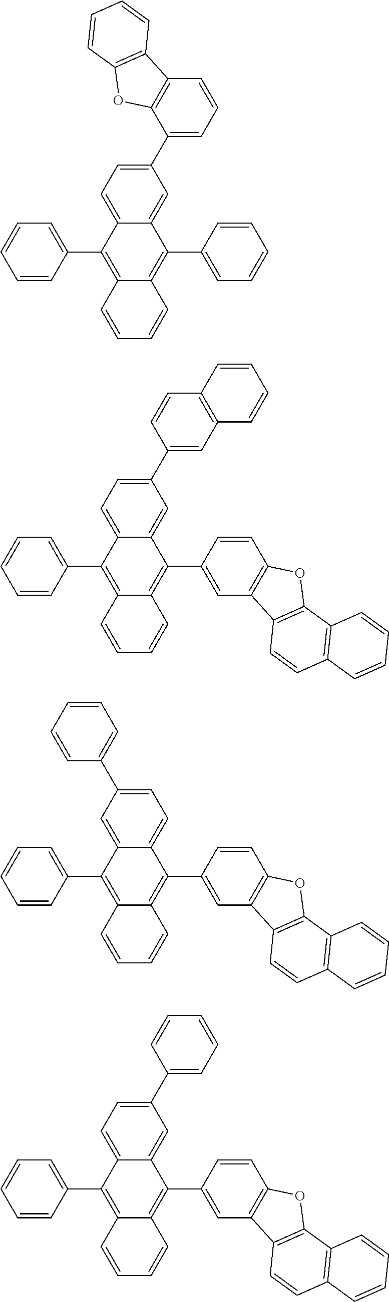 [SMILES: c1ccc(-c2c3ccccc3c(-c3ccccc3)c3cc(-c4cccc5c4oc4ccccc45)ccc23)cc1 ; c1ccc(-c2c3ccccc3c(-c3ccc4oc5c6ccccc6ccc5c4c3)c3cc(-c4ccc5ccccc5c4)ccc23)cc1 ; c1ccc(-c2ccc3c(-c4ccc5oc6c7ccccc7ccc6c5c4)c4ccccc4c(-c4ccccc4)c3c2)cc1 ; c1ccc(-c2ccc3c(-c4ccccc4)c4ccccc4c(-c4ccc5oc6c7ccccc7ccc6c5c4)c3c2)cc1]

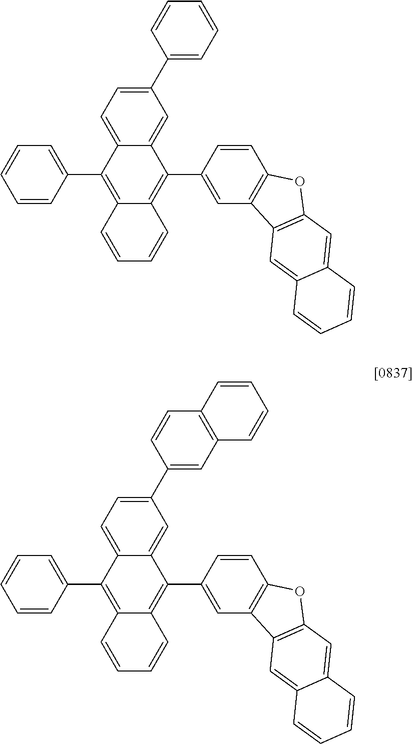 [SMILES: c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1]

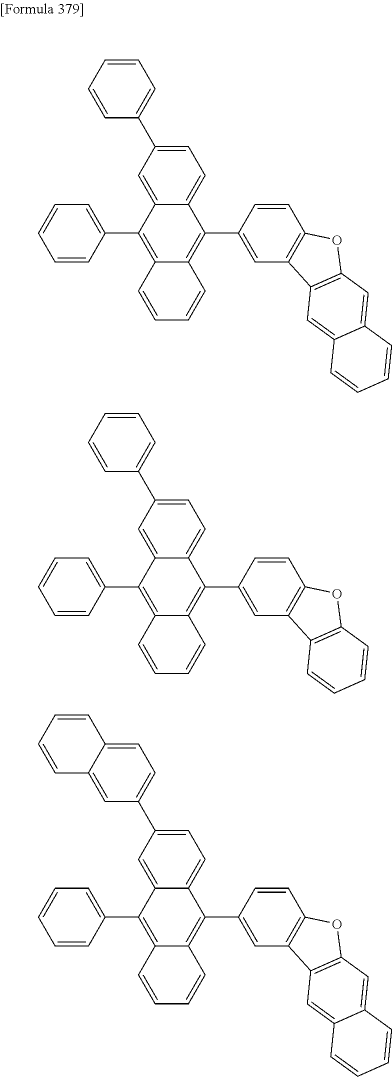 [SMILES: c1ccc(-c2ccc3c(-c4ccc5oc6cc7ccccc7cc6c5c4)c4ccccc4c(-c4ccccc4)c3c2)cc1 ; c1ccc(-c2ccc3c(-c4ccc5oc6ccccc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1]

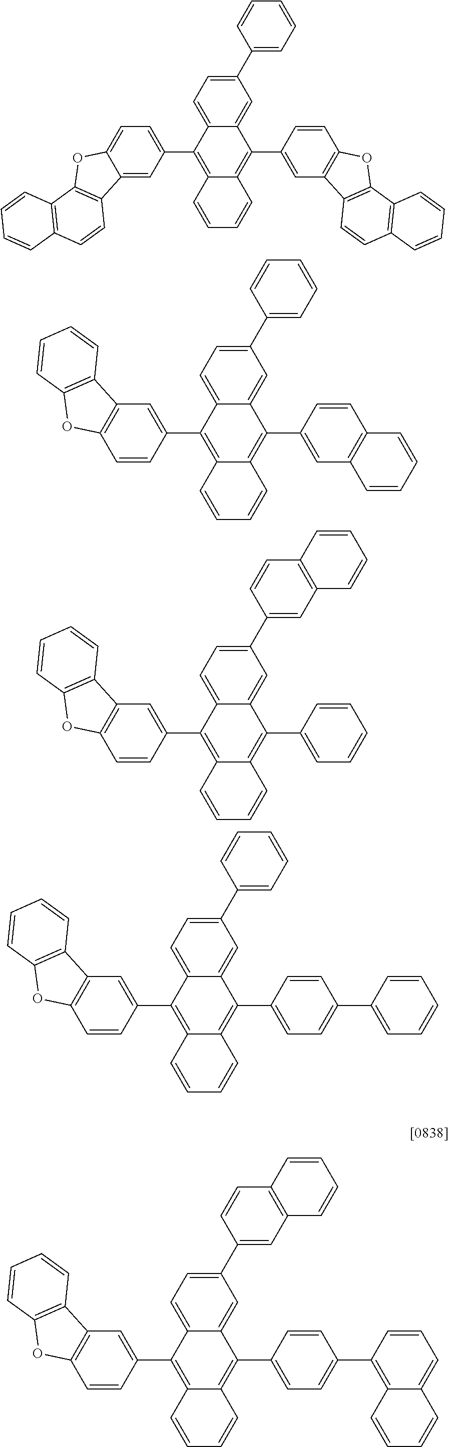

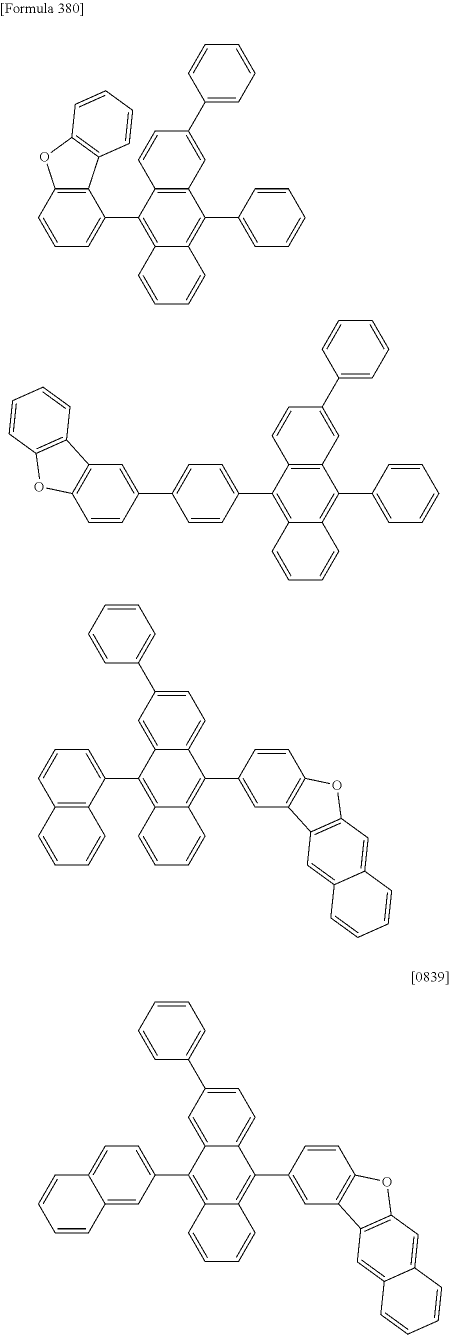 [SMILES: c1ccc(-c2ccc3c(-c4cccc5oc6ccccc6c45)c4ccccc4c(-c4ccccc4)c3c2)cc1 ; c1ccc(-c2ccc3c(-c4ccc(-c5ccc6oc7ccccc7c6c5)cc4)c4ccccc4c(-c4ccccc4)c3c2)cc1 ; c1ccc(-c2ccc3c(-c4ccc5oc6cc7ccccc7cc6c5c4)c4ccccc4c(-c4cccc5ccccc45)c3c2)cc1 ; c1ccc(-c2ccc3c(-c4ccc5oc6cc7ccccc7cc6c5c4)c4ccccc4c(-c4ccc5ccccc5c4)c3c2)cc1]

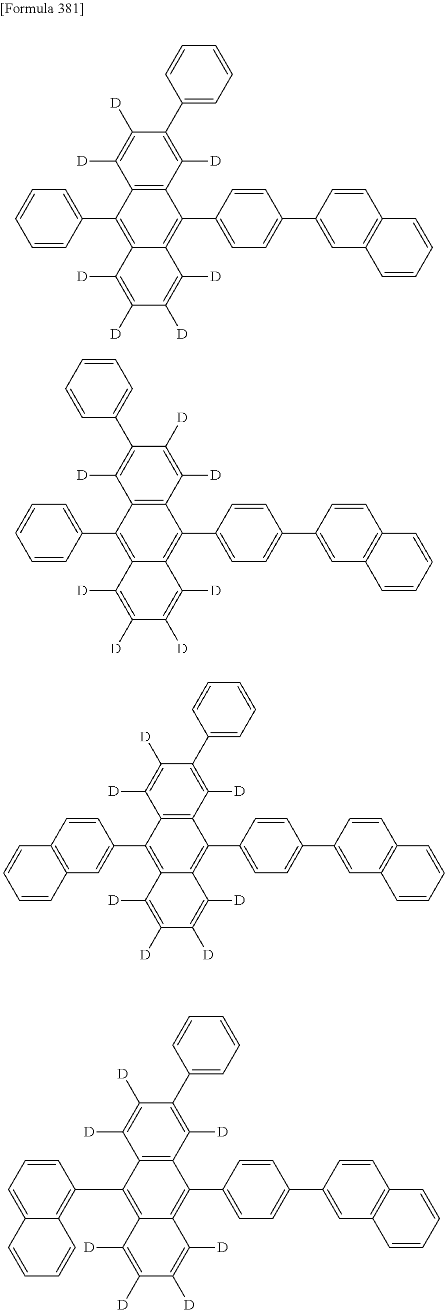

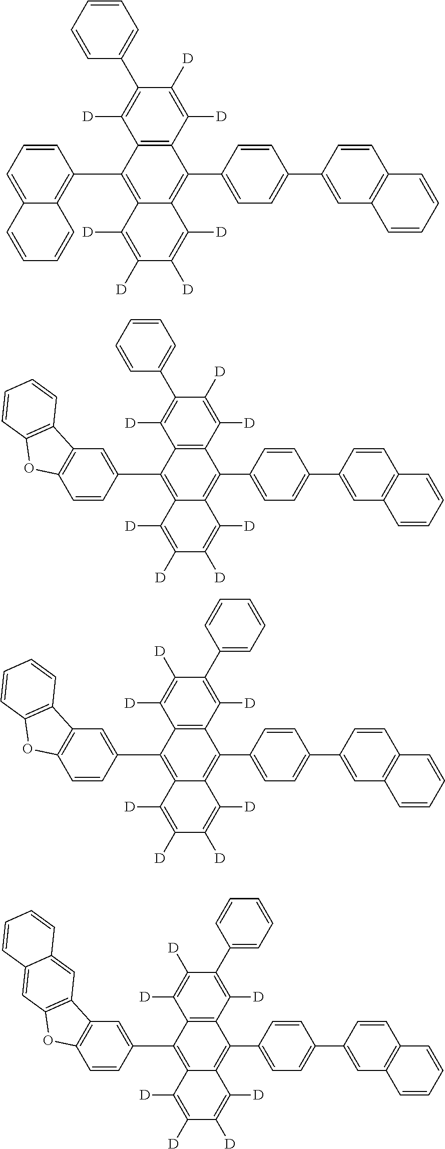

Fluorescent Compound (Compound M1)

In the organic EL device according to the sixth exemplary embodiment, preferably, the first emitting layer contains the compound according to the first exemplary embodiment (the compound represented by the formula (12X)) as a first host material and also contains a fluorescent compound (the compound M1).

In the organic EL device according to the sixth exemplary embodiment, preferably, the first emitting layer contains the compound according to the second exemplary embodiment (the compound represented by the formula (120)) as a first host material and also contains a fluorescent compound (the compound M1).

In the organic EL device according to the sixth exemplary embodiment, preferably, the first emitting layer contains the compound according to the third exemplary embodiment (at least one of compounds represented by one of the formulae (1) to (3)) as a first host material and also contains a fluorescent compound (the compound M1).

The fluorescent compound (the compound M1) represents the same as the compound M1 described in the fifth exemplary embodiment. Thus, in the organic EL device according to the sixth exemplary embodiment, the fluorescent compound (the compound M1) is preferably at least one compound selected from the group consisting of the compounds represented by the formula (100), the compounds represented by the formula (3), the compounds represented by the formula (4), the compounds represented by the formula (5), the compounds represented by the formula (6), the compounds represented by the formula (7), the compounds represented by the formula (8), the compounds represented by the formula (9), and the compounds represented by the formula (10).

In the organic EL device according to the sixth exemplary embodiment, when the first emitting layer contains the compound according to the first exemplary embodiment (the compound represented by the formula (12X)), the compound according to the second exemplary embodiment (the compound represented by the formula (120)), or the compound according to the third exemplary embodiment (at least one of compounds represented by one of the formulae (1) to (3)) as the first compound and a fluorescent compound (the compound M1), the first compound is preferably a host material (sometimes referred to as a matrix material), and the fluorescent compound (the compound M1) is preferably a dopant material (sometimes referred to as a guest material, an emitter, or a luminescent material).

In the organic EL device according to the sixth exemplary embodiment, when the first emitting layer contains the compound according to the first exemplary embodiment (the compound represented by the formula (12X)), the compound according to the second exemplary embodiment (the compound represented by the formula (120)), or the compound according to the third exemplary embodiment (at least one of compounds represented by one of the formulae (1) to (3)) as the first compound and a fluorescent compound (the compound M1), the singlet energy S₁(H1) of the first compound and the singlet energy S₁(M1) of the fluorescent compound preferably satisfy the relationship of a numerical formula (Numerical Formula 1) below.

S₁(H1)>S₁(M1) (Numerical Formula 1)

In the organic EL device according to the sixth exemplary embodiment, when the second emitting layer contains the second compound and a fluorescent compound (the compound M1), the singlet energy S₁(H2) of the second compound and the singlet energy S₁(M1) of the fluorescent compound (the compound M1) preferably satisfy a numerical formula (Numerical Formula 2) below.

S₁(H2)>S₁(M1) (Numerical Formula 2)

### Seventh Exemplary Embodiment

### Electronic Device

An electronic device according to a seventh exemplary embodiment is installed with any one of the organic EL devices according to the above exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited by the above-described exemplary embodiments but includes any modification and improvement as long as such modification and improvement are compatible with the invention.

For instance, the number of emitting layers is not limited to two, and more than two emitting layers may be provided and laminated with each other. When an organic EL device has more than two emitting layers, it is sufficient that at least one emitting layer contains a compound represented by the formula (120) (one example of the first compound). For example, the other emitting layer(s) may be a fluorescent emitting layer or a phosphorescent emitting layer utilizing light emission due to a direct electronic transition from a triplet excited state to the ground state.

When the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, or excitons.

For instance, when the blocking layer is provided in contact with the side of the emitting layer close to the cathode, the blocking layer permits transport of electrons and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded to a blocking layer.

Specific structure, shape and the like of the components in the invention may be designed in any manner as long as an object of the invention can be achieved.

### EXAMPLES

### Preparation 1 and Evaluation 1 of Organic EL Device

### Compound

A compound represented by the formula (120) according to Example 1 has the following structure.

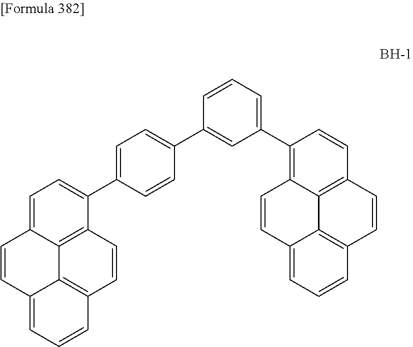 [SMILES: c1cc(-c2ccc(-c3ccc4ccc5cccc6ccc3c4c56)cc2)cc(-c2ccc3ccc4cccc5ccc2c3c45)c1]

Other compounds used to produce organic EL devices according to Example 1 and Comparative Example 1 have the following structures.

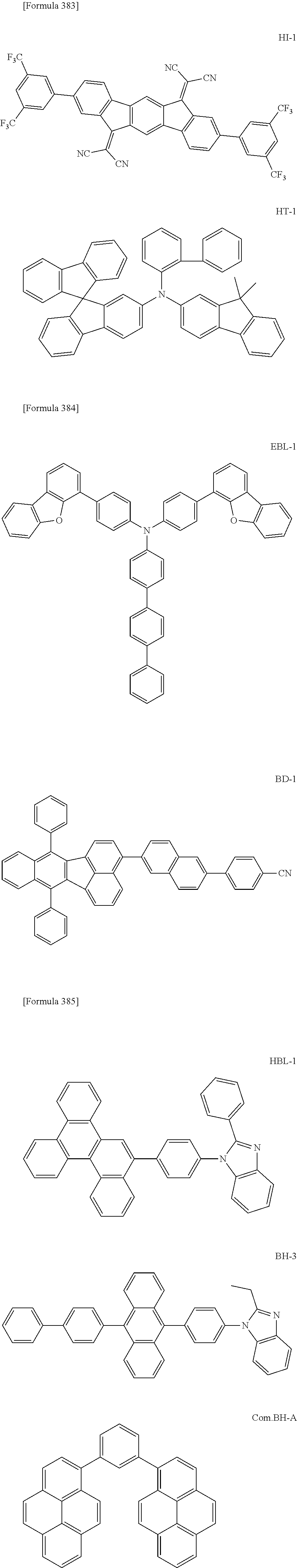

Production 1 of Organic EL device

An organic EL device was produced and evaluated as described below.

### Example 1

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The ITO transparent electrode had a thickness of 130 nm.

The cleaned glass substrate having the transparent electrode line was attached to a substrate holder of a vacuum deposition apparatus. First, a compound HI-1 was deposited on a surface, on which the transparent electrode line was formed, to cover the transparent electrode, thus forming a hole injecting layer (HI) with a thickness of 5 nm.

After the formation of the hole injecting layer, the compound HT-1 was deposited to form a first hole transporting layer (HT) with a thickness of 80 nm.

After the formation of the first hole transporting layer, a compound EBL-1 was deposited to form a second hole transporting layer (also referred to as an electron blocking layer) (EBL) with a thickness of 10 nm.

A compound BH-1 (host material (BH)) and a compound BD-1 (dopant material (BD)) were co-deposited on the second hole transporting layer such that the ratio of the compound BD-1 accounted for 4% by mass, thus forming an emitting layer with a thickness of 25 nm.

A compound HBL-1 was deposited on the emitting layer to form a first electron transporting layer (also referred to as a hole blocking layer) (HBL) with a thickness of 10 nm.

A compound BH-3 was deposited on the first electron transporting layer to form a second electron transporting layer (ET) with a thickness of 15 nm.

LiF was deposited on the second electron transporting layer to form an electron injecting layer with a thickness of 1 nm.

Metal Al was deposited on the electron injecting layer to form a cathode with a thickness of 50 nm.

A device arrangement of an organic EL device in Example 1 is roughly shown as follows.

ITO (130)/HI-1 (5)/HT-1 (80)/EBL-1 (10)/BH-1:BD-1 (25, 96%:4%)/HBL-1 (10)/BH-3 (15)/LiF (1)/Al (50)

The numerals in parentheses indicate the film thicknesses (unit: nm).

The numerals (96%:4%) represented by percentage in the same parentheses indicate a ratio (% by mass) of the host material (the compound BH-1) to the compound BD-1 in the emitting layer. Similar notations apply to the description below.

### Comparative Example 1

An organic EL device according to Comparative Example 1 was produced in the same manner as in Example 1 except that the compound BH-1 in the emitting layer in Example 1 was replaced with the compound listed in Table 1.

Evaluation 1 of Organic EL Device

The organic EL devices produced in Example 1 and Comparative Example 1 were evaluated as described below. Table 1 shows the evaluation results.

External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

Main Peak Wavelength λp when Device is Driven

Voltage was applied on the organic EL devices so that a current density of the organic EL device was 10 mA/cm², where spectral radiance spectrum was measured by a spectroradiometer CS-2000 (manufactured by Konica Minolta, Inc.). The main peak wavelength λp (unit: nm) was calculated based on the obtained spectral radiance spectrum.

TABLE 1

| | Emitting layer | | | | |
|---|---|---|---|---|---|
| | Host material | Dopant | Thickness | EQE | λp |
| | (BH) | material (BD) | [nm] | [%] | [nm] |
| Example 1 | BH-1 | BD-1 | 25 | 9.5 | 455 |
| Comparative | Com. BH-A | BD-1 | 25 | 8.9 | 455 |
| Example 1 | | | | | |

Table 1 shows that the organic EL device according to Example 1 containing the compound BH-1 as a host material in the emitting layer emitted light with a higher luminous efficiency than the organic EL device according to Comparative Example 1 containing a compound Com. BH-A as a host material in the emitting layer.

Preparation of Toluene Solution

The compound BD-1 was dissolved in toluene at a concentration of 4.9×10⁻⁶ mol/L to prepare a toluene solution of the compound BD-1.

Measurement of Fluorescent Main Peak Wavelength (FL-Peak)

A fluorescence spectrum measuring apparatus (fluorescence spectrophotometer F-7000 (manufactured by Hitachi High-Tech Science Corporation)) was used to measure a fluorescent main peak wavelength when the toluene solution of the compound BD-1 was excited at 390 nm.

The compound BD-1 had a fluorescent main peak wavelength at 442 nm.

### Preparation 2 and Evaluation 2 of Organic EL Device

### Compound

A compound represented by the formula (120) in Example 2 has the following structure.

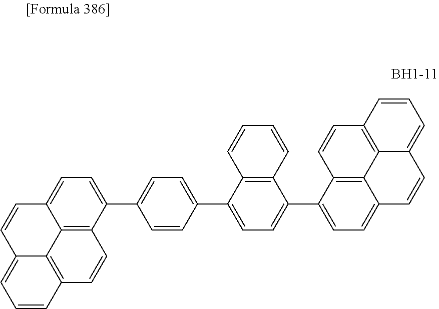 [SMILES: c1cc2ccc3ccc(-c4ccc(-c5ccc(-c6ccc7ccc8cccc9ccc6c7c89)c6ccccc56)cc4)c4ccc(c1)c2c34]

Other compounds used to produce an organic EL device in Example 2 have the following structures.

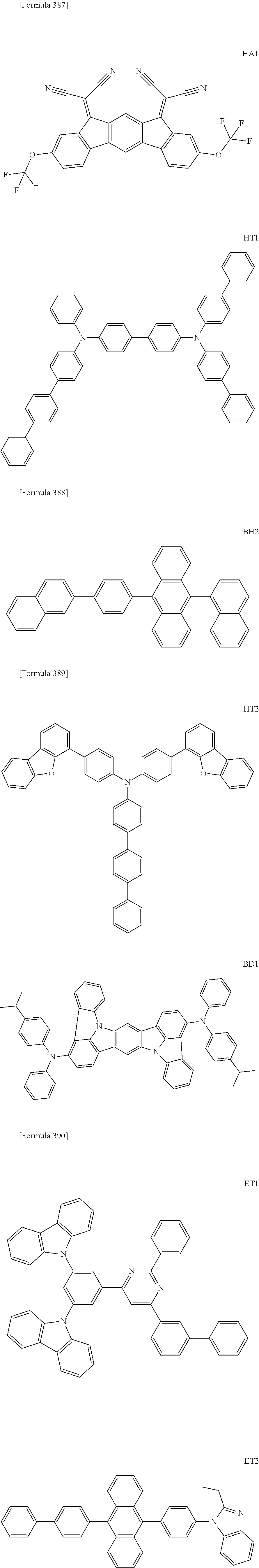

Production 2 of Organic EL Device

An organic EL device was produced and evaluated as described below.

### Example 2

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The ITO transparent electrode had a thickness of 130 nm.

The cleaned glass substrate having the transparent electrode line was attached to a substrate holder of a vacuum deposition apparatus. First, a compound HA1 was deposited on a surface, on which the transparent electrode line was formed, to cover the transparent electrode, thus forming a hole injecting layer (HI) with a thickness of 5 nm.

After the formation of the hole injecting layer, the compound HT1 was deposited to form a first hole transporting layer (HT) with a thickness of 80 nm.

After the formation of the first hole transporting layer, a compound HT2 was deposited to form a second hole transporting layer (also referred to as an electron blocking layer) (EBL) with a thickness of 10 nm.

A compound BH1-11 (first host material (BH)) and a compound BD1 (dopant material (BD)) were co-deposited on the second hole transporting layer such that the ratio of the compound BD1 accounted for 2% by mass, thus forming a first emitting layer with a thickness of 5 nm.

A compound BH2 (second host material (BH)) and the compound BD1 (dopant material (BD)) were co-deposited on the first emitting layer such that the ratio of the compound BD1 accounted for 2% by mass, thus forming a second emitting layer with a thickness of 20 nm.

A compound ET1 was deposited on the second emitting layer to form a first electron transporting layer (also referred to as a hole blocking layer) (HBL) with a thickness of 10 nm.

A compound ET2 was deposited on the first electron transporting layer to form a second electron transporting layer (ET) with a thickness of 15 nm.

LiF was deposited on the second electron transporting layer to form an electron injecting layer with a thickness of 1 nm.

Metal Al was deposited on the electron injecting layer to form a cathode with a thickness of 80 nm.

A device arrangement of an organic EL device in Example 2 is roughly shown as follows.

ITO (130)/HA1 (5)/HT1 (80)/HT2 (10)/BH1-11:BD1 (5, 98%:2%)/BH2:BD1 (20, 98%:2%)/ET1 (10)/ET2 (15)/LiF (1)/Al (80)

Evaluation 2 of Organic EL Device

The organic EL device produced in Example 2 was evaluated as described below. Table 2 shows the evaluation results.

External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

Life LT95

Voltage was applied on the resultant organic EL devices so that a current density was 50 mA/cm², where a time (LT95 (unit: hr)) elapsed before a luminance intensity was reduced to 95% of the initial luminance intensity was measured.

Drive Voltage

The voltage (unit: V) when electric current was applied between the anode and the cathode so that the current density was 10 mA/cm² was measured.

TABLE 2

| | First emitting layer | | | Second emitting layer | | | | | |
|---|---|---|---|---|---|---|---|---|---|
| | First | Fluorescent | Thickness | Second | Fluorescent | Thickness | Voltage | EQE | LT95 |
| | compound | compound | [nm] | compound | compound | [nm] | [V] | [%] | [hr] |
| Example 2 | BH1-11 | BD1 | 5 | BH2 | BD1 | 20 | 3.48 | 10.8 | 275 |

### Preparation 3 and Evaluation 3 of Organic EL Device

### Compound

Compounds represented by the formula (120) according to Examples 3 to 9 have the following structures.

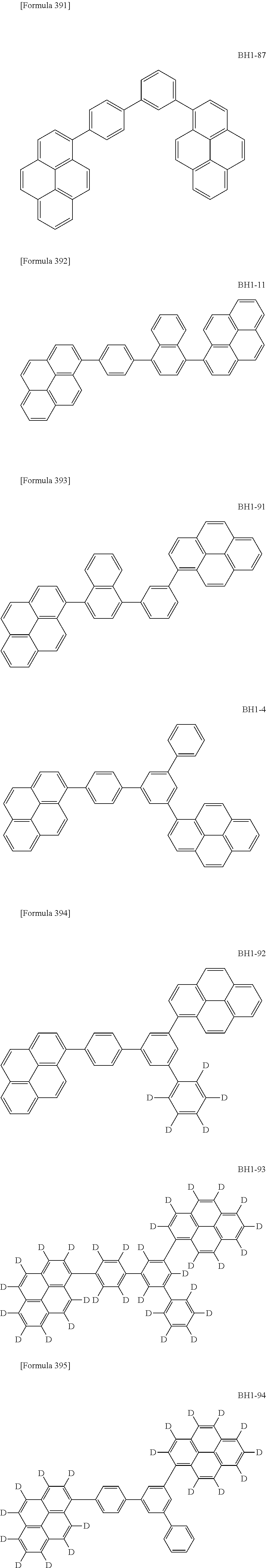

Other compounds used to produce organic EL devices according to Examples 3 to 9 have the following structures.

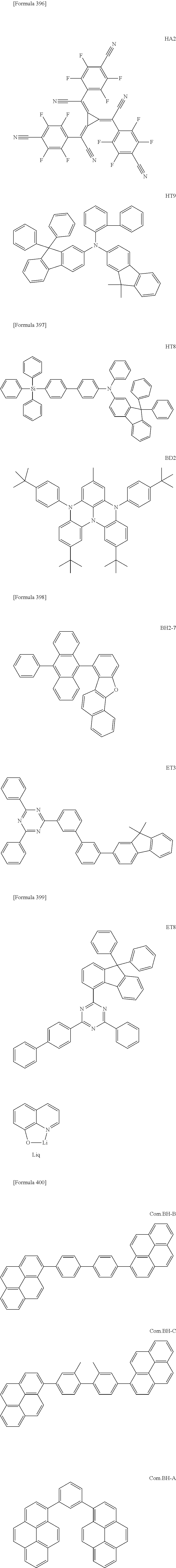

Production 3 of Organic EL Device

### Example 3

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The ITO transparent electrode had a thickness of 130 nm.

The cleaned glass substrate having the transparent electrode line was attached to a substrate holder of a vacuum deposition apparatus. First, a compound HT9 and a compound HA2 were co-deposited on a surface, on which the transparent electrode line was formed, to cover the transparent electrode, thus forming a hole injecting layer (HI) with a thickness of 10 nm. In the hole injecting layer, the ratio of the compound HT9 accounted for 90% by mass, and the ratio of the compound HA2 accounted for 10% by mass.

After the formation of the hole injecting layer, the compound HT9 was deposited to form a first hole transporting layer (HT) with a thickness of 85 nm.

After the formation of the first hole transporting layer, a compound HT8 was deposited to form a second hole transporting layer (also referred to as an electron blocking layer) (EBL) with a thickness of 5 nm.

A compound BH1-87 (first host material (BH)) and a compound BD2 (dopant material (BD)) were co-deposited on the second hole transporting layer such that the ratio of the compound BD2 accounted for 2% by mass, thus forming a first emitting layer with a thickness of 5 nm.

A compound BH2-7 (second host material (BH)) and the compound BD2 (dopant material (BD)) were co-deposited on the first emitting layer such that the ratio of the compound BD2 accounted for 2% by mass, thus forming a second emitting layer with a thickness of 15 nm.

A compound ET3 was deposited on the second emitting layer to form a first electron transporting layer (also referred to as a hole blocking layer) (HBL) with a thickness of 5 nm.

A compound ET8 and a compound Liq were co-deposited on the first electron transporting layer (HBL) to form a second electron transporting layer (ET) with a thickness of 25 nm. In the second electron transporting layer (ET), the ratio of the compound ET8 accounted for 50% by mass, and the ratio of the compound Liq accounted for 50% by mass. Liq is an abbreviation of (8-quinolinolato)lithium.

Liq was deposited on the second electron transporting layer to form an electron injecting layer with a thickness of 1 nm.

Metal Al was deposited on the electron injecting layer to form a cathode with a thickness of 80 nm.

A device arrangement of an organic EL device in Example 3 is roughly shown as follows.

ITO (130)/HT9:HA2 (10, 90%:10%)/HT9 (85)/HT8 (5)/BH1-87:BD2 (5, 98%:2%)/BH2-7:BD2 (15, 98%:2%)/ET3 (5)/ET8:Liq (25, 50%:50%)/Liq (1)/Al (80)

The numerals in parentheses indicate the film thicknesses (unit: nm).

The numerals (90%:10%) represented by percentage in the same parentheses indicate a ratio (% by mass) of the compound HT9 to the compound HA2 in the hole injecting layer.

The numerals (98%:2%) represented by percentage in the same parentheses indicate the ratio (% by mass) of the host material (compound BH1-87 or compound BH2-7) to the dopant material (compound BD2) in the first emitting layer or the second emitting layer. The numerals (50%:50%) represented by percentage in the same parentheses indicate the ratio (% by mass) of the compound ET8 to the compound Liq in the electron transporting layer (ET).

### Examples 4 to 9 and Comparative Examples 2 to 4

Organic EL devices according to Examples 4 to 9 and Comparative Examples 2 to 4 were produced in the same manner as in Example 3 except that the compound BH-87 in the emitting layer in Example 3 was replaced with the compounds listed in Table 3.

Evaluation 3 of Organic EL Device

The organic EL devices produced in Examples 3 to 8 and Comparative Examples 2 and 3 were evaluated as described below. For the organic EL devices produced in Example 9 and Comparative Example 4, only CIE1931 chromaticity was measured. Table 3 shows the evaluation results.

External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

Life LT95

Voltage was applied on the resultant organic EL devices so that a current density was 50 mA/cm², where a time (LT95 (unit: hr)) elapsed before a luminance intensity was reduced to 95% of the initial luminance intensity was measured.

Drive Voltage

The voltage (unit: V) when electric current was applied between the anode and the cathode so that the current density was 10 mA/cm² was measured.

CIE1931 Chromaticity

Voltage was applied on the organic EL devices so that a current density was 10 mA/cm², where spectral radiance spectrum was measured by a spectroradiometer (CS-2000 manufactured by Konica Minolta, Inc.).

From the obtained spectral radiance spectrum, CIEx and CIEy were calculated.

TABLE 3
Explanation of Table 3

| | First emitting layer | | | Second emitting layer | | | | | | | |
|---|---|---|---|---|---|---|---|---|---|---|---|
| | First | Fluorescent | Thickness | Second | Fluorescent | Thickness | Voltage | EQE | LT95 | | |
| | compound | compound | [nm] | compound | compound | [nm] | [V] | [%] | [hr] | CIE-x | CIE-y |
| Example 3 | BH1-87 | BD2 | 5 | BH2-7 | BD2 | 15 | 3.22 | 10.4 | 142 | 0.137 | 0.084 |
| Example 4 | BH1-91 | BD2 | 5 | BH2-7 | BD2 | 15 | 3.22 | 10.2 | 135 | 0.137 | 0.084 |
| Example 5 | BH1-4 | BD2 | 5 | BH2-7 | BD2 | 15 | 3.24 | 10.1 | 140 | 0.136 | 0.084 |
| Example 6 | BH1-92 | BD2 | 5 | BH2-7 | BD2 | 15 | 3.23 | 10.1 | 152 | 0.136 | 0.086 |
| Example 7 | BH1-93 | BD2 | 5 | BH2-7 | BD2 | 15 | 3.23 | 10.2 | 165 | 0.137 | 0.084 |
| Example 8 | BH1-94 | BD2 | 5 | BH2-7 | BD2 | 15 | 3.22 | 10.1 | 156 | 0.136 | 0.085 |
| Example 9 | BH1-11 | BD2 | 5 | BH2- | BD2 | 15 | — | — | — | 0.138 | 0.083 |
| Comparative | Com.BH-B | BD2 | 5 | BH2-7 | BD2 | 15 | 3.23 | 8.1 | 80 | 0.144 | 0.113 |
| Example 2 | | | | | | | | | | | |
| Comparative | Com.BH-C | BD2 | 5 | BH2-7 | BD2 | 15 | 3.26 | 8.0 | 45 | 0.143 | 0.110 |
| Example 3 | | | | | | | | | | | |
| Comparative | Com.BH-A | BD2 | 5 | BH2-7 | BD2 | 15 | — | — | — | 0.144 | 0.112 |
| Example 4 | | | | | | | | | | | |

“—” indicates no measurement.

Table 3 shows that Examples 3 to 8 containing a compound represented by the formula (120) as a host material in the emitting layer emitted light with a higher luminous efficiency than Comparative Examples 2 and 3 containing compounds Com.BH-B and Com.BH-C as a host material in the emitting layer.

Examples 3 to 9 containing a compound represented by the formula (120) as a host material in the emitting layer showed that deterioration in chromaticity was suppressed as compared with Comparative Examples 2 to 4 containing compounds Com.BH-B, Com.BH-C, and Com.BH-A as a host material in the emitting layer.

### Preparation 4 and Evaluation 4 of Organic EL Device

### Compound

A compound represented by the formula (2) according to Example 1A has the following structure.

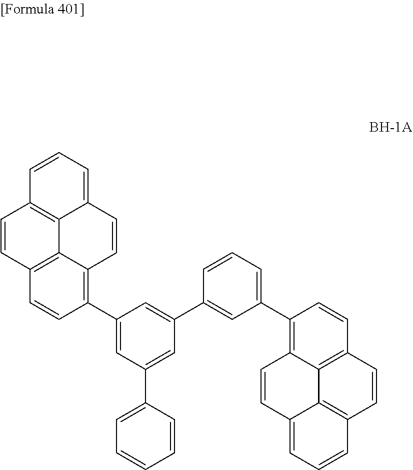 [SMILES: c1ccc(-c2cc(-c3cccc(-c4ccc5ccc6cccc7ccc4c5c67)c3)cc(-c3ccc4ccc5cccc6ccc3c4c56)c2)cc1]

Other compounds used to produce organic EL devices according to Example 1A and Comparative Example 1A have the following structures.

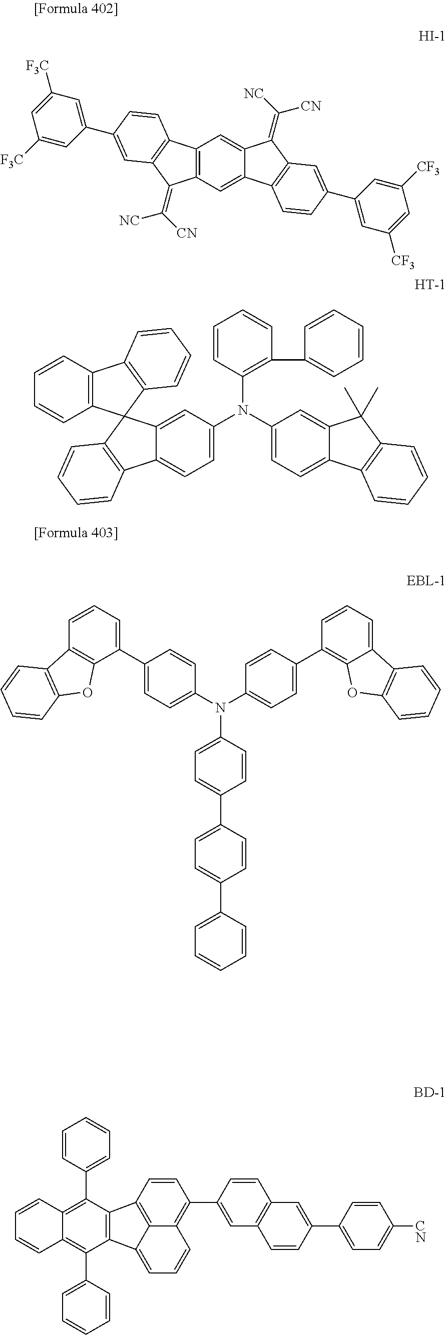 [SMILES: N#CC(C#N)=C1c2cc(-c3cc(C(F)(F)F)cc(C(F)(F)F)c3)ccc2-c2cc3c(cc21)-c1ccc(-c2cc(C(F)(F)F)cc(C(F)(F)F)c2)cc1C3=C(C#N)C#N ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; N#Cc1ccc(-c2ccc3cc(-c4ccc5c6c(cccc46)-c4c-5c(-c5ccccc5)c5ccccc5c4-c4ccccc4)ccc3c2)cc1]

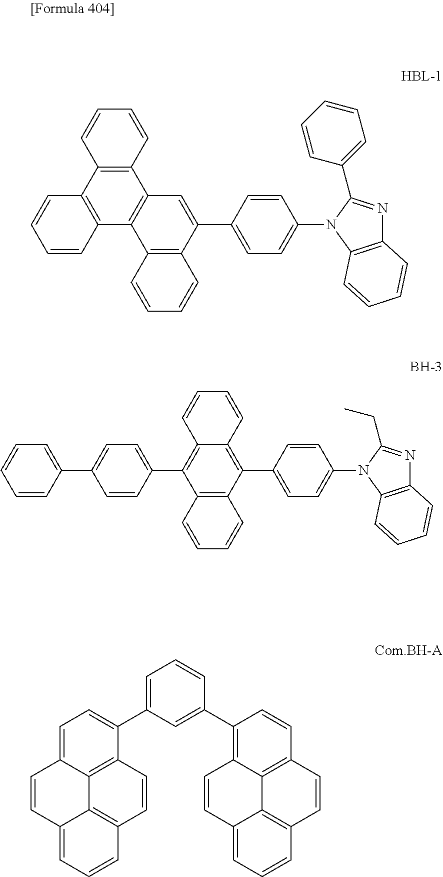

Production 4 of Organic EL Device

An organic EL device was produced and evaluated as described below.

### Example 1A

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The ITO transparent electrode had a thickness of 130 nm.

The cleaned glass substrate having the transparent electrode line was attached to a substrate holder of a vacuum deposition apparatus. First, a compound HI-1 was deposited on a surface on which the transparent electrode line was formed and covered the transparent electrode, thus forming a hole injecting layer (HI) with a thickness of 5 nm.

After the formation of the hole injecting layer, the compound HT-1 was deposited to form a first hole transporting layer (HT) with a thickness of 80 nm.

After the formation of the first hole transporting layer, a compound EBL-1 was deposited to form a second hole transporting layer (also referred to as an electron blocking layer) (EBL) with a thickness of 10 nm.

A compound BH-1A (host material (BH)) and a compound BD-1 (dopant material (BD)) were co-deposited on the second hole transporting layer such that the ratio of the compound BD-1 accounted for 4% by mass, thus forming an emitting layer with a thickness of 25 nm.

A compound HBL-1 was deposited on the emitting layer to form a first electron transporting layer (also referred to as a hole blocking layer) (HBL) with a thickness of 10 nm.

A compound BH-3 was deposited on the first electron transporting layer to form a second electron transporting layer (ET) with a thickness of 15 nm.

LiF was deposited on the second electron transporting layer to form an electron injecting layer with a thickness of 1 nm.

Metal Al was deposited on the electron injecting layer to form a cathode with a thickness of 50 nm.

A device arrangement of an organic EL device in Example 1A is roughly shown as follows.

ITO (130)/HI-1 (5)/HT-1 (80)/EBL-1 (10)/BH-1A:BD-1 (25, 96%:4%)/HBL-1 (10)/BH-3 (15)/LiF (1)/Al (50)

The numerals in parentheses indicate the film thicknesses (unit: nm).

The numerals (96%:4%) represented by percentage in the same parentheses indicate a ratio (% by mass) of the host material (the compound BH-1A) to the compound BD-1 in the emitting layer. Similar notations apply to the description below.

### Comparative Example 1A

An organic EL device according to Comparative Example 1A was produced in the same manner as in Example 1A except that the compound BH-1A in the emitting layer in Example 1A was replaced with the compound listed in Table 4.

Evaluation 4 of Organic EL Device

The organic EL devices produced in Example 1A and Comparative Example 1A were evaluated as described below. Table 4 shows the evaluation results.

External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

Main Peak Wavelength λp while Driving Device

Voltage was applied on the organic EL devices so that a current density was 10 mA/cm², where spectral radiance spectrum was measured by a spectroradiometer (CS-2000 manufactured by Konica Minolta, Inc.). The main peak wavelength λp (unit: nm) was calculated from the obtained spectral radiance spectrum.

TABLE 4

| | Emitting layer | | | | |
|---|---|---|---|---|---|
| | Host material | Dopant | Thickness | EQE | λp |
| | (BH) | material (BD) | [nm] | [%] | [nm] |
| Example 1A | BH-1A | BD-1 | 25 | 9.6 | 455 |
| Comparative | Com. BH-A | BD-1 | 25 | 8.9 | 455 |
| Example 1A | | | | | |

Table 4 shows that the organic EL device according to Example 1A containing the compound BH-1A as a host material in the emitting layer emitted light with a higher luminous efficiency than the organic EL device according to Comparative Example 1A containing the compound Com.BH-A as a host material in the emitting layer.

Preparation of Toluene Solution

The compound BD-1 was dissolved in toluene at a concentration of 4.9×10⁻⁶ mol/L to prepare a toluene solution of the compound BD-1.

Measurement of Fluorescent Main Peak Wavelength (FL-Peak)

A fluorescence spectrum measuring apparatus (fluorescence spectrophotometer F-7000 (manufactured by Hitachi High-Tech Science Corporation)) was used to measure a fluorescent main peak wavelength when the toluene solution of the compound BD-1 was excited at 390 nm.

The compound BD-1 had a fluorescent main peak wavelength at 442 nm.

### Preparation 5 and Evaluation 5 of Organic EL Device

Compound

Compounds represented by the formula (1) or (3) according to Examples 2A to 8A have the following structures.

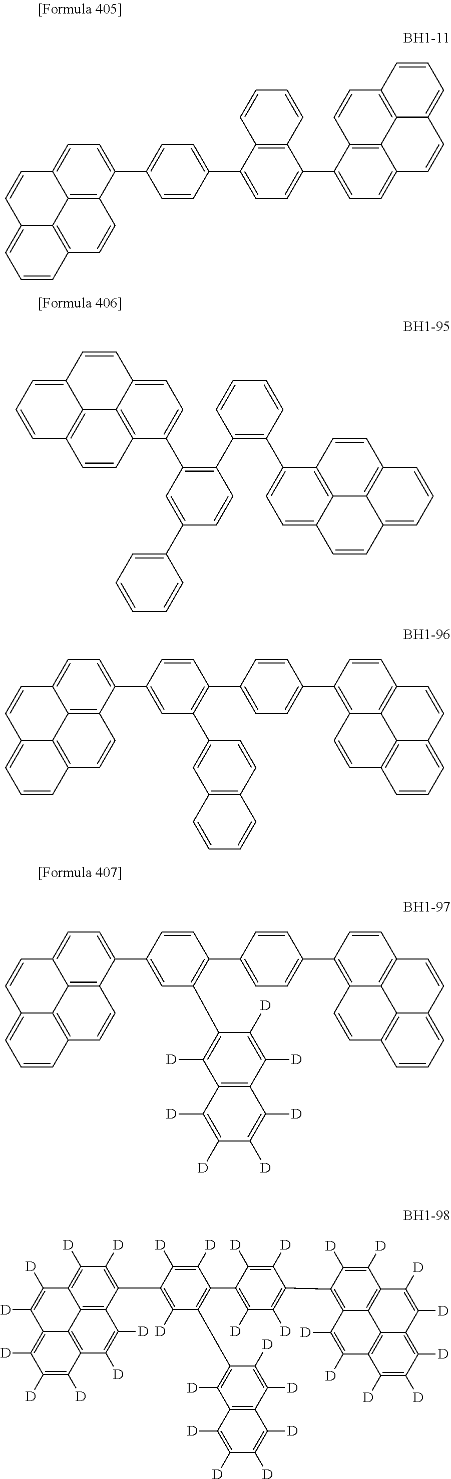

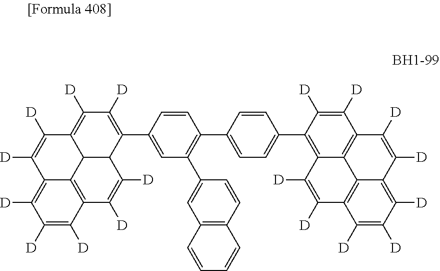 [SMILES: [2H]C1=C([2H])c2c([2H])c([2H])c([2H])c3c2C2C1=C([2H])C([2H])=C(c1ccc(-c4ccc(-c5c([2H])c([2H])c6c([2H])c([2H])c7c([2H])c([2H])c([2H])c8c([2H])c([2H])c5c6c78)cc4)c(-c4ccc5ccccc5c4)c1)C2C([2H])=C3[2H]]

Other compounds used to produce organic EL devices according to Examples 2A to 8A have the following structures.

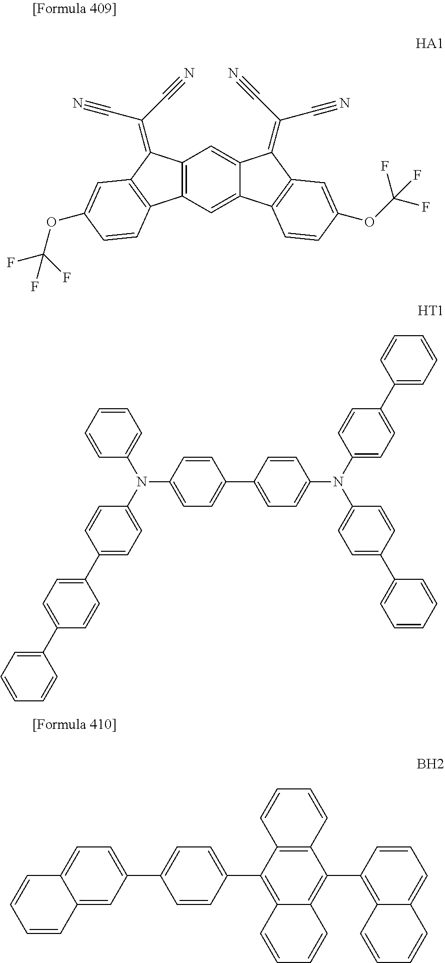

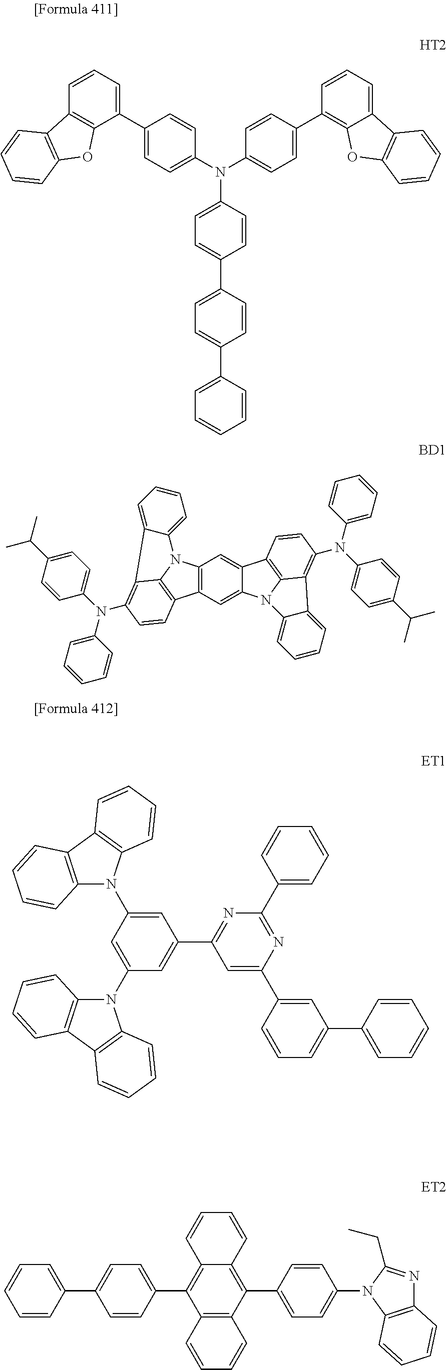 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; CC(C)c1ccc(N(c2ccccc2)c2ccc3c4cc5c(cc4n4c6ccccc6c2c34)c2ccc(N(c3ccccc3)c3ccc(C(C)C)cc3)c3c4ccccc4n5c23)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

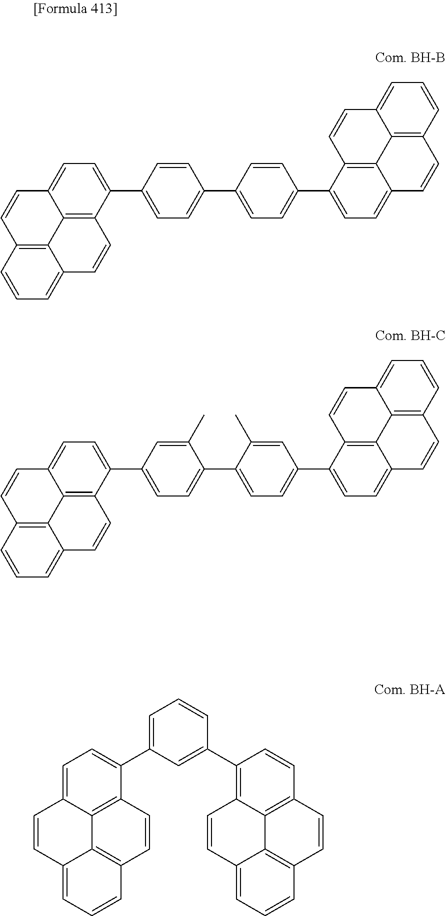

Production 5 of Organic EL Device

An organic EL device was produced and evaluated as described below.

### Example 2A

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The ITO transparent electrode had a thickness of 130 nm.

The cleaned glass substrate having the transparent electrode line was attached to a substrate holder of a vacuum deposition apparatus. First, a compound HA1 was deposited on a surface on which the transparent electrode line was formed and covered the transparent electrode, thus forming a hole injecting layer (HI) with a thickness of 5 nm.

After the formation of the hole injecting layer, the compound HT1 was deposited to form a first hole transporting layer (HT) with a thickness of 80 nm.

After the formation of the first hole transporting layer, a compound HT2 was deposited to form a second hole transporting layer (also referred to as an electron blocking layer) (EBL) with a thickness of 10 nm.

A compound BH1-11 (first host material (BH)) and a compound BD1 (dopant material (BD)) were co-deposited on the second hole transporting layer such that the ratio of the compound BD1 accounted for 2% by mass, thus forming a first emitting layer with a thickness of 5 nm.

A compound BH2 (second host material (BH)) and the compound BD1 (dopant material (BD)) were co-deposited on the first emitting layer such that the ratio of the compound BD1 accounted for 2% by mass, thus forming a second emitting layer with a thickness of 20 nm.

A compound ET1 was deposited on the second emitting layer to form a first electron transporting layer (also referred to as a hole blocking layer) (HBL) with a thickness of 10 nm.

A compound ET2 was deposited on the first electron transporting layer to form a second electron transporting layer (ET) with a thickness of 15 nm.

LiF was deposited on the second electron transporting layer to form an electron injecting layer with a thickness of 1 nm.

Metal Al was deposited on the electron injecting layer to form a cathode with a thickness of 80 nm.

A device arrangement of an organic EL device in Example 2A is roughly shown as follows.

ITO (130)/HA1 (5)/HT1 (80)/HT2 (10)/BH1-11:BD1 (5, 98%:2%)/BH2:BD1 (20, 98%:2%)/ET1 (10)/ET2 (15)/LiF (1)/Al (80)

### Examples 3A to 8A and Comparative Examples 2A to 4A

Organic EL devices according to Examples 3A to 8A and Comparative Examples 2A to 4A were produced in the same manner as in Example 2A except that the compound BH1-11 in the emitting layer in Example 2A was replaced with the compounds listed in Table 5.

Evaluation 5 of Organic EL Device

The organic EL devices produced in Examples 2A to 7A and Comparative Examples 2A and 3A were evaluated as described below. For the organic EL devices produced in Example 8A and Comparative Example 4A, only CIE1931 chromaticity was measured. Table 5 shows the evaluation results.

External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

Life LT95

Voltage was applied on the resultant organic EL devices so that a current density was 50 mA/cm², where a time (LT95 (unit: hr)) elapsed before a luminance intensity was reduced to 95% of the initial luminance intensity was measured.

Drive Voltage

The voltage (unit: V) when electric current was applied between the anode and the cathode so that the current density was 10 mA/cm² was measured.

TABLE 5
Explanation of Table 5

| | First emitting layer | | | Second emitting layer | | | | | | | |
|---|---|---|---|---|---|---|---|---|---|---|---|
| | First | Fluorescent | Thickness | Second | Fluorescent | Thickness | Voltage | EQE | LT95 | | |
| | compound | compound | [nm] | compound | compound | [nm] | [V] | [%] | [hr] | CIE-x | CIE-y |
| Example 2A | BH1-11 | BD1 | 5 | BH2 | BD1 | 20 | 3.48 | 10.8 | 275 | 0.139 | 0.084 |
| Example 3A | BH1-95 | BD1 | 5 | BH2 | BD1 | 20 | 3.45 | 10.5 | 265 | 0.138 | 0.083 |
| Example 4A | BH1-96 | BD1 | 5 | BH2 | BD1 | 20 | 3.44 | 10.4 | 250 | 0.135 | 0.082 |
| Example 5A | BH1-97 | BD1 | 5 | BH2 | BD1 | 20 | 3.43 | 10.5 | 285 | 0.136 | 0.086 |
| Example 6A | BH1-98 | BD1 | 5 | BH2 | BD1 | 20 | 3.43 | 10.4 | 305 | 0.137 | 0.084 |
| Example 7A | BH1-99 | BD1 | 5 | BH2 | BD1 | 20 | 3.42 | 10.3 | 295 | 0.136 | 0.084 |
| Example 8A | BH1-87 | BD1 | 5 | BH2 | BD1 | 20 | — | — | — | 0.138 | 0.086 |
| Comparative | Com.BH-B | BD1 | 5 | BH2 | BD1 | 20 | 3.45 | 8.1 | 120 | 0.145 | 0.114 |
| Example 2A | | | | | | | | | | | |
| Comparative | Com.BH-C | BD1 | 5 | BH2 | BD1 | 20 | 3.51 | 8.0 | 100 | 0.143 | 0.112 |
| Example 3A | | | | | | | | | | | |
| Comparative | Com.BH-A | BD1 | 5 | BH2 | BD1 | 20 | — | — | — | 0.143 | 0.114 |
| Example 4A | | | | | | | | | | | |

“—” indicates no measurement.

Table 5 shows that Examples 3A to 8A containing a compound represented by the formula (1) or (3) as a host material in the emitting layer emitted light with a higher luminous efficiency than Comparative Examples 2A and 3A containing compounds Com.BH-B and Com.BH-C as a host material in the emitting layer.

Examples 3A to 9A containing a compound represented by the formula (1) or (3) as a host material in the emitting layer showed that deterioration in chromaticity was suppressed as compared with Comparative Examples 2A to 4A containing compounds Com.BH-B, Com.BH-C, and Com.BH-A as a host material in the emitting layer.

### EXPLANATION OF CODES

1, 1A organic EL device, 2 substrate, 3 anode, 4 cathode, 5 emitting layer, 51 first emitting layer, 52 second emitting layer, 6 hole injecting layer, 7 hole transporting layer, 8 electron transporting layer, 9 electron injecting layer, 10, 10A organic layer.

1. A compound represented by a formula (12X) below,
Py₁-L₁-L₂-Py₂ (12X)
where:
Py₁ and Py₂ each independently represent
a substituted or unsubstituted 1-pyrenyl group,
L₁ and L₂ each independently represent
a substituted or unsubstituted phenylene group, or
a substituted or unsubstituted naphthylene group,
when L₁ and L₂ each independently represent a substituted or unsubstituted phenylene group, -L₁-L₂- in the formula (12X) represents a group represented by one of formulae (13-1) to (13-6), (10-1), (20-1), and (30-1) below,
when L₁ and L₂ each independently represent a substituted or unsubstituted naphthylene group, a bonding position of the naphthylene group as L₁ is different from a bonding position of the naphthylene group as L₂,
when Py₁ and Py₂ each independently represent a substituted 1-pyrenyl group, a substituent E for the substituted 1-pyrenyl group each independently represent a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms,
Rx, Ry, and Rz in the —Si(Rx)(Ry)(Rz) each independently represent a substituted or unsubstituted alkyl group having 1 to 50 carbon atoms, or a substituted or unsubstituted aryl group having 6 to 50 ring carbon atoms, and
when a plurality of substituents E are present, the plurality of substituents E are mutually the same or different;
a substituent F for the substituent E that is substituted or unsubstituted is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group, 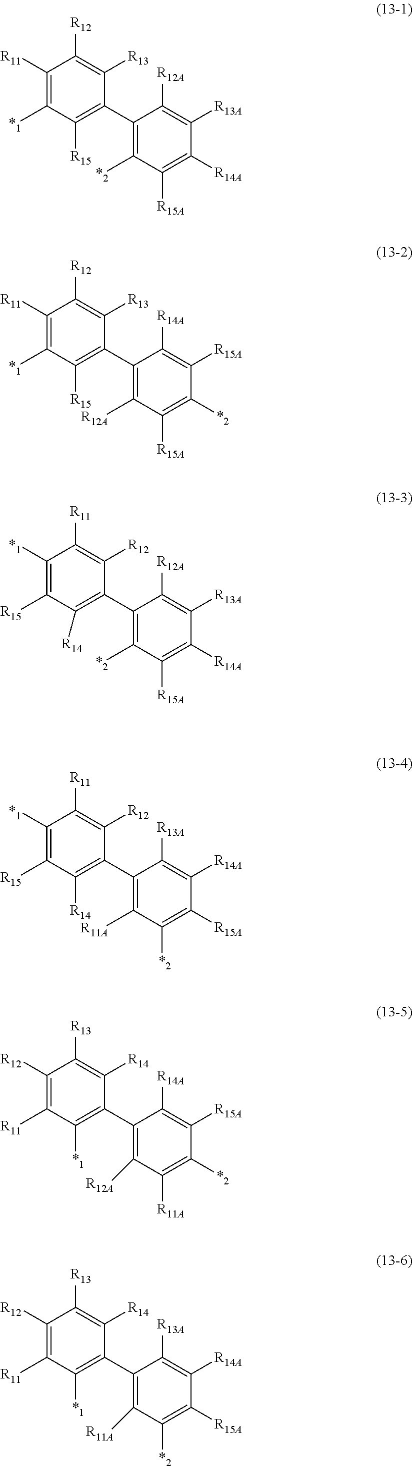
where: R₁₁ to R₁₅ and R_{11A} to R_{15A} each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in —Si(Rx)(Ry)(Rz) each independently represent a substituted or unsubstituted alkyl group having 1 to 50 carbon atoms, or a substituted or unsubstituted aryl group having 6 to 50 ring carbon atoms;
a substituent for a substituted or unsubstituted group in R₁₁ to R₁₅ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group;
at least one combination of adjacent two or more of R₁₁ to R₁₅ are not mutually bonded to form no substituted or unsubstituted monocyclic ring or to form no substituted or unsubstituted fused ring;
at least one combination of adjacent two or more of R_{11A} to R_{15A} are not mutually bonded to form no substituted or unsubstituted monocyclic ring or to form no substituted or unsubstituted fused ring; and
*1 in the formulae (13-1) to (13-6) represents a bonding position to Py₁ in the formula (12X), and *2 represents a position of bonding to Py₂ in the formula (12X), 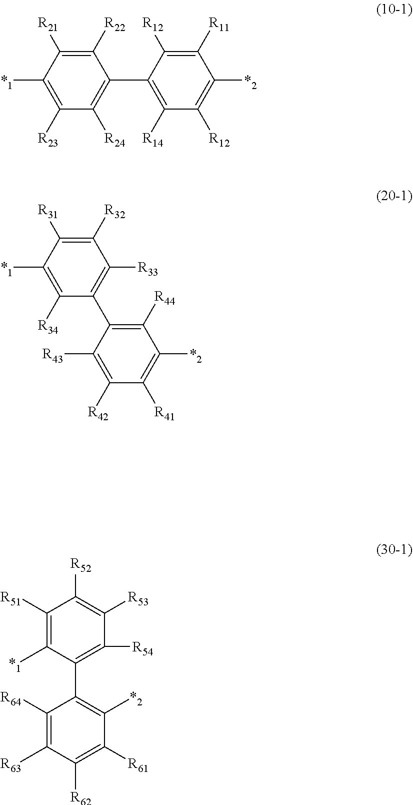
where: in the formula (10-1):
at least one combination of a combination of R₁₁ and R₁₂, a combination of R₁₃ and R₁₄, a combination of R₂₁ and R₂₂, or a combination of R₂₃ and R₂₄ are mutually bonded to form a substituted or unsubstituted monocyclic ring, are mutually bonded to form a substituted or unsubstituted fused ring, or are not mutually bonded;
R₁₁, R₁₃, R₂₁, and R₂₃ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by a formula (4) below,
R₁₂, R₁₄, R₂₂, and R₂₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the formula (4) below, and
at least one of R₁₁ to R₁₄ or R₂₁ to R₂₄ is not a hydrogen atom,
where: in the formulae (20-1) and (30-1):
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring, are mutually bonded to form a substituted or unsubstituted fused ring, or are not mutually bonded;
R₃₁ to R₃₄, R₄₁ to R₄₄, R₅₁ to R₅₄, and R₆₁ to R₆₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted spirobifluorenyl group, or a group represented by the formula (4) below,
at least one of R₃₁ to R₃₄ or R₄₁ to R₄₄ is not a hydrogen atom, and
at least one of R₅₁ to R₅₄ or R₆₁ to R₆₄ is not a hydrogen atom,
in a compound represented by the formula (10-1), a combination of R₁₁ and R₁₃ and a combination of R₂₁ and R₂₃ are different combinations, or a combination of R₁₂ and R₁₄ and a combination of R₂₂ and R₂₄ are different combinations,
in a compound represented by the formula (20-1), R₃₁ is different from R₄₁, R₃₂ is different from R₄₂, R₃₃ is different from R₄₃, or R₃₄ is different from R₄₄,
in a compound represented by the formula (30-1), R₅₁ is different from R₆₁, R₅₂ is different from R₆₂, R₅₃ is different from R₆₃, or R₅₄ is different from R₆₄, and
*1 in the formulae (10-1), (20-1), and (30-1) represents a bonding position to Py₁ in the formula (12X), and *2 represents a position of bonding to Py₂ in the formula (12X), 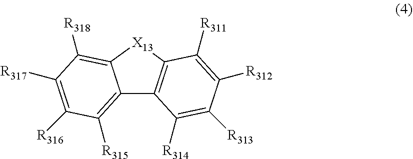
where: X₁₃ represents an oxygen atom, a sulfur atom, or NR₃₁₉,
at least one combination of adjacent two or more of R₃₁₁ to R₃₁₈ are mutually bonded to form a substituted or unsubstituted monocyclic ring, are mutually bonded to form a substituted or unsubstituted fused ring, or are not mutually bonded;
R₃₁₁ to R₃₁₉ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group; and
any one of R₃₁₁ to R₃₁₉ that do not form the substituted or unsubstituted monocyclic ring and do not form the substituted or unsubstituted fused ring is a single bond bonded to at least one of R₁₁ to R₁₄, R₂₁ to R₂₄, R₃₁ to R₃₄, R₄₁ to R₄₄, R₅₁ to R₅₄, or R₆₁ to R₆₄;
in the formulae (10-1), (20-1), and (30-1) and the formula (4), a substituent for a substituted or unsubstituted group in R₁₁ to R₁₄, R₂₁ to R₂₄, R₃₁ to R₃₄, R₄₁ to R₄₄, R₅₁ to R₅₄, R₆₁ to R₆₄, and R₃₁₁ to R₃₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

2. The compound according to claim 1, wherein
a compound represented by the formula (12X) is represented by a formula (120) below, 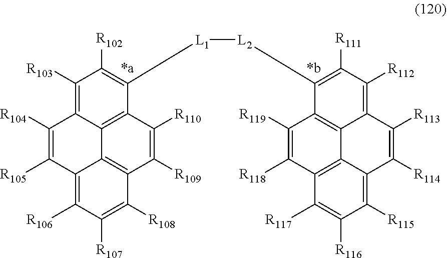
where: L₁, L₂, a substituted or unsubstituted 1-pyrenyl group having R₁₀₂ to R₁₁₀, a substituted or unsubstituted 1-pyrenyl group having R₁₁₁ to R₁₁₉ each independently represent the same as Py₁ and Py₂ in the formula (12X),
L₁ represents a group represented by one of formulae (11) to (13) below, and L₂ represents a group represented by one of formulae (11A) to (13A) below;
R₁₀₂ to R₁₁₉ each independently represent a hydrogen atom, a substituted or unsubstituted alkyl group having 1 to 50 carbon atoms, a substituted or unsubstituted cycloalkyl group having 3 to 50 carbon atoms,
a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in —Si(Rx)(Ry)(Rz) each independently represent a substituted or unsubstituted alkyl group having 1 to 50 carbon atoms, or a substituted or unsubstituted aryl group having 6 to 50 ring carbon atoms,
a substituent for a substituted or unsubstituted group in R₁₀₂ to R₁₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group, 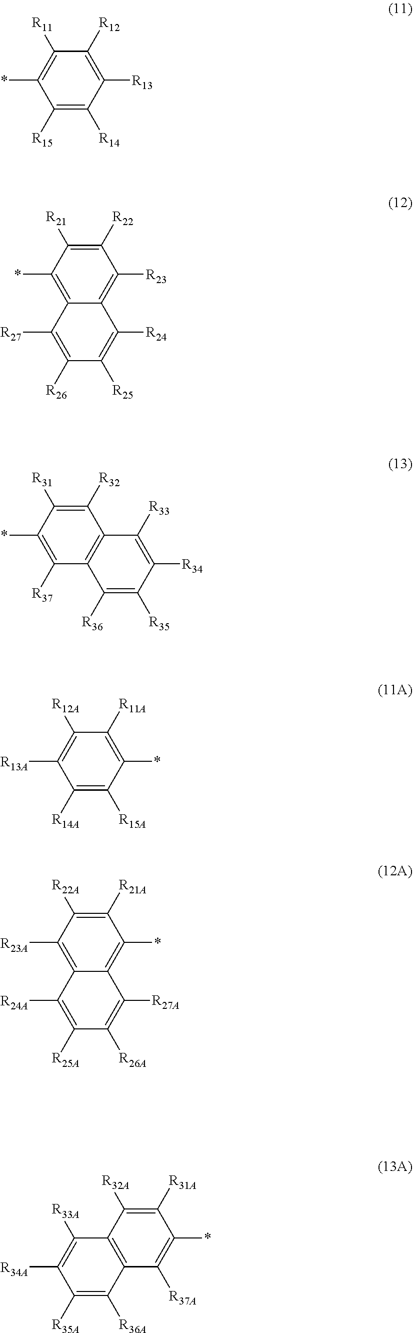
where: in the formulae (11) to (13) and (11A) to (13A): R₁₁ to R₁₅, R₂₁ to R₂₇, R₃₁ to R₃₇, R_{11A} to R_{15A}, R_{21A} to R_{27A}, and R_{31A} to R_{37A} each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in —Si(Rx)(Ry)(Rz) each independently represent
a substituted or unsubstituted alkyl group having 1 to 50 carbon atoms, or a substituted or unsubstituted aryl group having 6 to 50 ring carbon atoms;
a substituent for a substituted or unsubstituted group in R₁₁ to R₁₅, R₂₁ to R₂₇, and R₃₁ to R₃₇ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group;
at least one combination of adjacent two or more of R₁₁ to R₁₅ are not mutually bonded to form no substituted or unsubstituted monocyclic ring or to form no substituted or unsubstituted fused ring;
at least one combination of adjacent two or more of R_{11A} to R_{15A} are not mutually bonded to form no substituted or unsubstituted monocyclic ring or to form no substituted or unsubstituted fused ring;
at least one combination of adjacent two or more of R₂₁ to R₂₇ are not mutually bonded to form no substituted or unsubstituted monocyclic ring or to form no substituted or unsubstituted fused ring;
at least one combination of adjacent two or more of R_{21A} to R_{27A} are not mutually bonded to form no substituted or unsubstituted monocyclic ring or to form no substituted or unsubstituted fused ring;
at least one combination of adjacent two or more of R₃₁ to R₃₇ are not mutually bonded to form no substituted or unsubstituted monocyclic ring or to form no substituted or unsubstituted fused ring;
at least one combination of adjacent two or more of R_{31A} to R_{37A} are not mutually bonded to form no substituted or unsubstituted monocyclic ring or to form no substituted or unsubstituted fused ring;
in the formulae (11) to (13) representing L₁, * represents a bonding position to *a in the formula (120), and any one of R₁₁ to R₁₅, R₂₁ to R₂₇, and R₃₁ to R₃₇ is a single bond bonded to L₂;
in the formulae (11A) to (13A) representing L₂, * represents a position of bonding to *b in the formula (120), and any one of R_{11A} to R_{15A}, R_{21A} to R_{27A}, and R_{31A} to R_{37A} is a single bond bonded to L₁;
when R₁₂ or R₁₄ in L₁ is a single bond bonded to L₂, one of R_{11A}, R_{13A}, R_{15A}, R_{21A} to R_{27A}, and R_{31A} to R_{37A} in L₂ is a single bond bonded to L₁;
when R₁₁ or R₁₅ in L₁ is a single bond bonded to L₂, one of R_{12A}, R_{13A}, R_{14A}, R_{21A} to R_{27A}, and R_{31A} to R_{37A} in L₂ is a single bond bonded to L₁;
when R₁₃ in L₁ is a single bond bonded to L₂, one of R_{11A}, R_{12A}, R_{14A}, R_{15A}, R_{21A} to R_{27A}, and R_{31A} to R_{37A} in L₂ is a single bond bonded to L₁;
when R₂₁ in L₁ is a single bond bonded to L₂, one of R_{11A} to R_{15A}, R_{22A} to R_{27A}, and R_{31A} to R_{37A} in L₂ is a single bond bonded to L₁;
when R₂₂ in L₁ is a single bond bonded to L₂, one of R_{11A} to R_{15A}, R_{21A}, R_{23A} to R_{27A}, and R_{31A} to R_{37A} in L₂ is a single bond bonded to L₁;
when R₂₃ in L₁ is a single bond bonded to L₂, one of R_{11A} to R_{15A}, R_{21A} to R_{22A}, R_{24A} to R_{27A}, and R_{31A} to R_{37A} in L₂ is a single bond bonded to L₁;
when R₂₄ in L₁ is a single bond bonded to L₂, one of R_{11A} to R_{15A}, R_{21A} to R_{23A}, R_{25A} to R_{27A}, and R_{31A} to R_{37A} in L₂ is a single bond bonded to L₁;
when R₂₅ in L₁ is a single bond bonded to L₂, one of R_{11A} to R_{15A}, R_{21A} to R_{24A}, R_{26A} to R_{27A}, and R_{31A} to R_{37A} in L₂ is a single bond bonded to L₁;
when R₂₆ in L₁ is a single bond bonded to L₂, one of R_{11A} to R_{15A}, R_{21A} to R_{25A}, R_{27A}, and R_{31A} to R_{37A} in L₂ is a single bond bonded to L₁;
when R₂₇ in L₁ is a single bond bonded to L₂, one of R_{11A} to R_{15A}, R_{21A} to R_{26A}, and R_{31A} to R_{37A} in L₂ is a single bond bonded to L₁;
when R₃₁ in L₁ is a single bond bonded to L₂, one of R_{11A} to R_{15A}, R_{21A} to R_{27A}, and R_{32A} to R_{37A} in L₂ is a single bond bonded to L₁;
when R₃₂ in L₁ is a single bond bonded to L₂, one of R_{11A} to R_{15A}, R_{21A} to R_{27A}, R_{31A}, and R_{33A} to R_{37A} in L₂ is a single bond bonded to L₁;
when R₃₃ in L₁ is a single bond bonded to L₂, one of R_{11A} to R_{15A}, R_{21A} to R_{27A}, R_{31A} to R_{32A}, and R_{34A} to R_{37A} in L₂ is a single bond bonded to L₁;
when R₃₄ in L₁ is a single bond bonded to L₂, one of R_{11A} to R_{15A}, R_{21A} to R_{27A}, R_{31A} to R_{33A}, and R_{35A} to R_{37A} in L₂ is a single bond bonded to L₁;
when R₃₅ in L₁ is a single bond bonded to L₂, one of R_{11A} to R_{15A}, R_{21A} to R_{27A}, R_{31A} to R_{34A}, and R_{36A} to R_{37A} in L₂ is a single bond bonded to L₁;
when R₃₆ in L₁ is a single bond bonded to L₂, one of R_{11A} to R_{15A}, R_{21A} to R_{27A}, R_{31A} to R_{35A}, and R_{37A} in L₂ is a single bond bonded to L₁; and
when R₃₇ in L₁ is a single bond bonded to L₂, one of R_{11A} to R_{15A}, R_{21A} to R_{27A}, and R_{31A} to R_{36A} in L₂ is a single bond bonded to L₁.

3. The compound according to claim 1, wherein
a compound represented by the formula (12X) is represented by a formula (1), (2), or (3) below, 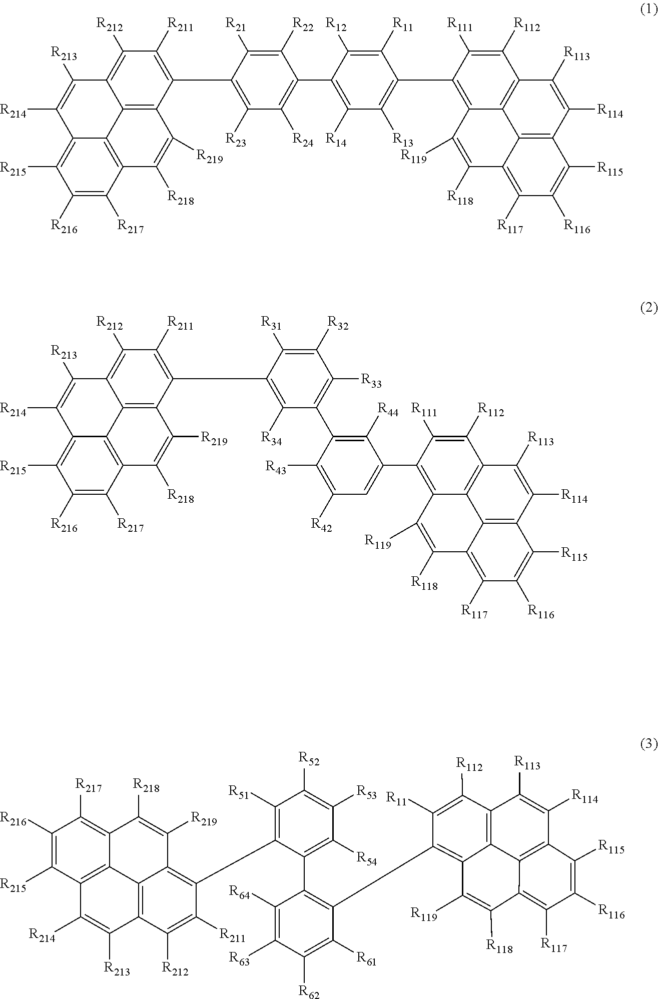
where: a substituted or unsubstituted 1-pyrenyl group having R₂₁₁ to R₂₁₉ represents the same as the substituent E for the substituted 1-pyrenyl group in the formula (12X), and a substituted or unsubstituted 1-pyrenyl group having R₁₁₁ to R₁₁₉ represents the same as the substituent E for the substituted 1-pyrenyl group in the formula (12X);
a substituted or unsubstituted biphenylene group having R₁₁ to R₁₄ and R₂₁ to R₂₄ in the formula (1), a substituted or unsubstituted biphenylene group with R₃₁ to R₃₄ and R₄₁ to R₄₄ in the formula (2), and a substituted or unsubstituted biphenylene group having R₅₁ to R₅₄ and R₆₁ to R₆₄ in the formula (3) each independently represent the same as -L₁-L₂- in the formula (12X);
R₁₁₁ to R₁₁₉ and R₂₁₁ to R₂₁₉ each independently represent a hydrogen atom, a substituted or unsubstituted alkyl group having 1 to 50 carbon atoms, a substituted or unsubstituted cycloalkyl group having 3 to 50 carbon atoms,
a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in —Si(Rx)(Ry)(Rz) each independently represent a substituted or unsubstituted alkyl group having 1 to 50 carbon atoms, or a substituted or unsubstituted aryl group having 6 to 50 ring carbon atoms,
in the formula (1):
at least one combination of a combination of R₁ and R₁₂, a combination of R₁₃ and R₁₄, a combination of R₂₁ and R₂₂, or a combination of R₂₃ and R₂₄ are mutually bonded to form a substituted or unsubstituted monocyclic ring, are mutually bonded to form a substituted or unsubstituted fused ring, or are not mutually bonded;
R₁₁, R₁₃, R₂₁, and R₂₃ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the formula (4) in the formulae (10-1), (20-1), and (30-1);
R₁₂, R₁₄, R₂₂, and R₂₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the formula (4) in the formulae (10-1), (20-1), and (30-1); and
at least one of R₁₁ to R₁₄ or R₂₁ to R₂₄ is not a hydrogen atom,
in the formulae (2) to (3):
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring, are mutually bonded to form a substituted or unsubstituted fused ring, or are not mutually bonded;
R₃₁ to R₃₄, R₄₁ to R₄₄, R₅₁ to R₅₄, and R₆₁ to R₆₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, substituted or unsubstituted spirobifluorenyl group, or a group represented by the following formula (4);
at least one of R₃₁ to R₃₄ or R₄₁ to R₄₄ is not a hydrogen atom; and
at least one of R₅₁ to R₅₄ or R₆₁ to R₆₄ is not a hydrogen atom;
in a compound represented by the formula (1), a combination of R₁₁ and R₁₃ and a combination of R₂₁ and R₂₃ are different combinations, or a combination of R₁₂ and R₁₄ and a combination of R₂₂ and R₂₄ are different combinations;
in a compound represented by the formula (2), a combination of R₃₁ and R₄₁ is different from at least one combination of a combination of R₃₂ and R₄₂, a combination of R₃₃ and R₄₃, or a combination of R₃₄ and R₄₄;
in a compound represented by the formula (3), a combination of R₅₁ and R₆₁ is different from at least one combination of a combination of R₅₂ and R₆₂, a combination of R₅₃ and R₆₃, or a combination of R₅₄ and R₆₄;
in the formulae (1) to (3):
a substituent for a substituted or unsubstituted group in R₁₁ to R₁₄, R₂₁ to R₂₄, R₃₁ to R₃₄, R₄₁ to R₄₄, R₅₁ to R₅₄, R₆₁ to R₆₄, R₁₁₁ to R₁₁₉, R₂₁₁ to R₂₁₉, and R₃₁₁ to R₃₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

4. An organic electroluminescence device comprising:
an anode;
a cathode; and
an emitting layer between the anode and the cathode,
wherein the emitting layer comprises the compound according to claim 1 as a host material.

5. (canceled)

6. A compound represented by a formula (120) below, 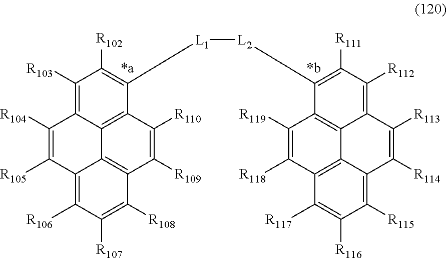
where: L₁ represents a group represented by one of formulae (11) to (13) below, and L₂ represents a group represented by one of formulae (11A) to (13A) below,
R₁₀₂ to R₁₁₉ each independently represent a hydrogen atom, a substituted or unsubstituted alkyl group having 1 to 50 carbon atoms, a substituted or unsubstituted cycloalkyl group having 3 to 50 carbon atoms,
a group represented by —Si(Rx)(Ry)(Rz), a substituted or unsubstituted aryl group having 6 to 50 ring carbon atoms, or a substituted or unsubstituted heterocyclic group having 5 to 50 ring atoms;
Rx, Ry, and Rz in —Si(Rx)(Ry)(Rz) each independently represent a substituted or unsubstituted alkyl group having 1 to 50 carbon atoms, or a substituted or unsubstituted aryl group having 6 to 50 ring carbon atoms;
a substituent for a substituted or unsubstituted group in R₁₀₂ to R₁₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group, 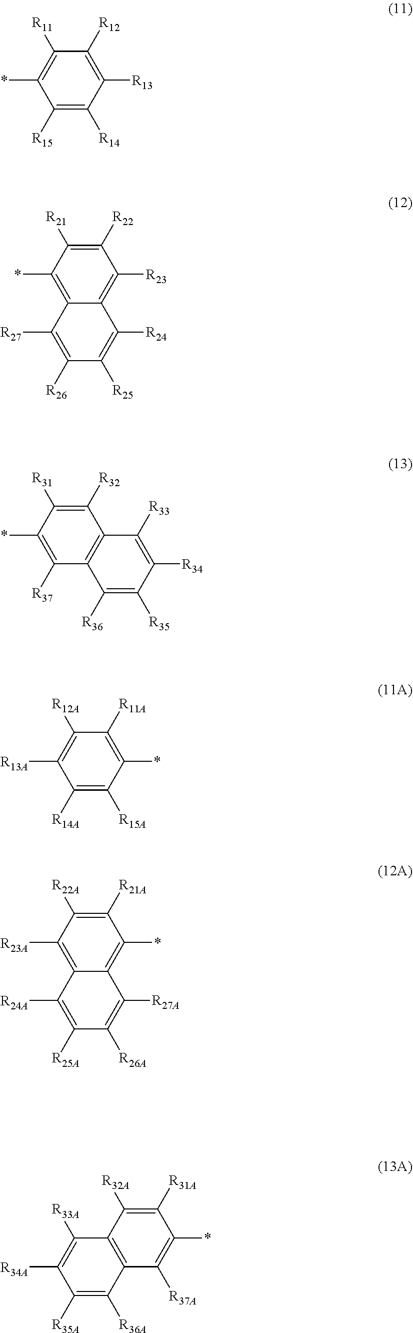
where: in the formulae (11) to (13) and (11A) to (13A), R₁₁ to R₁₅, R₂₁ to R₂₇, R₃₁ to R₃₇, R_{11A} to R_{15A}, R_{21A} to R_{27A}, and R_{31A} to R_{37A} each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms,
Rx, Ry, and Rz in —Si(Rx)(Ry)(Rz) each independently represent a substituted or unsubstituted alkyl group having 1 to 50 carbon atoms, or a substituted or unsubstituted aryl group having 6 to 50 ring carbon atoms;
a substituent for a substituted or unsubstituted group in R₁₁ to R₁₅, R₂₁ to R₂₇, and R₃₁ to R₃₇ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group;
at least one combination of adjacent two or more of R₁₁ to R₁₅ are not mutually bonded to form no substituted or unsubstituted monocyclic ring or to form no substituted or unsubstituted fused ring;
at least one combination of adjacent two or more of R_{11A} to R_{15A} are not mutually bonded to form no substituted or unsubstituted monocyclic ring or to form no substituted or unsubstituted fused ring;
at least one combination of adjacent two or more of R₂₁ to R₂₇ are not mutually bonded to form no substituted or unsubstituted monocyclic ring or to form no substituted or unsubstituted fused ring;
at least one combination of adjacent two or more of R_{21A} to R_{27A} are not mutually bonded to form no substituted or unsubstituted monocyclic ring or to form no substituted or unsubstituted fused ring;
at least one combination of adjacent two or more of R₃₁ to R₃₇ are not mutually bonded to form no substituted or unsubstituted monocyclic ring or to form no substituted or unsubstituted fused ring;
at least one combination of adjacent two or more of R_{31A} to R_{37A} are not mutually bonded to form no substituted or unsubstituted monocyclic ring or to form no substituted or unsubstituted fused ring;
in L₁ represented by the formulae (11) to (13), * represents a bonding position to *a in the formula (120), and any one of R₁₁ to R₁₅, R₂₁ to R₂₇, and R₃₁ to R₃₇ is a single bond bonded to L₂;
in L₂ represented by the formulae (11A) to (13A), * represents a bonding position to *b in the formula (120), and any one of R_{11A} to R_{15A}, R_{21A} to R_{27A}, and R_{31A} to R_{37A} is a single bond bonded to L₁;
when R₁₂ or R₁₄ in L₁ is a single bond bonded to L₂, one of R_{11A}, R_{13A}, R_{15A}, R_{21A} to R_{27A}, and R_{31A} to R_{37A} in L₂ is a single bond bonded to L₁;
when R₁₁ or R₁₅ in L₁ is a single bond bonded to L₂, one of R_{12A}, R_{13A}, R_{14A}, R_{21A} to R_{27A}, and R_{31A} to R_{37A} in L₂ is a single bond bonded to L₁;
when R₁₃ in L₁ is a single bond bonded to L₂, one of R_{11A}, R_{12A}, R_{14A}, R_{15A}, R_{21A} to R_{27A}, and R_{31A} to R_{37A} in L₂ is a single bond bonded to L₁;
when R₂₁ in L₁ is a single bond bonded to L₂, one of R_{11A} to R_{15A}, R_{22A} to R_{27A}, and R_{31A} to R_{37A} in L₂ is a single bond bonded to L₁;
when R₂₂ in L₁ is a single bond bonded to L₂, one of R_{11A} to R_{15A}, R_{21A}, R_{23A} to R_{27A}, and R_{31A} to R_{37A} in L₂ is a single bond bonded to L₁;
when R₂₃ in L₁ is a single bond bonded to L₂, one of R_{11A} to R_{15A}, R_{21A} to R_{22A}, R_{24A} to R_{27A}, and R_{31A} to R_{37A} in L₂ is a single bond bonded to L₁;
when R₂₄ in L₁ is a single bond bonded to L₂, one of R_{11A} to R_{15A}, R_{21A} to R_{23A}, R_{25A} to R_{27A}, and R_{31A} to R_{37A} in L₂ is a single bond bonded to L₁;
when R₂₅ in L₁ is a single bond bonded to L₂, one of R_{11A} to R_{15A}, R_{21A} to R_{24A}, R_{26A} to R_{27A}, and R_{31A} to R_{37A} in L₂ is a single bond bonded to L₁;
when R₂₆ in L₁ is a single bond bonded to L₂, one of R_{11A} to R_{15A}, R_{21A} to R_{25A}, R_{27A}, and R_{31A} to R_{37A} in L₂ is a single bond bonded to L₁;
when R₂₇ in L₁ is a single bond bonded to L₂, one of R_{11A} to R_{15A}, R_{21A} to R_{26A}, and R_{31A} to R_{37A} in L₂ is a single bond bonded to L₁;
when R₃₁ in L₁ is a single bond bonded to L₂, one of R_{11A} to R_{15A}, R_{21A} to R_{27A}, and R_{32A} to R_{37A} in L₂ is a single bond bonded to L₁;
when R₃₂ in L₁ is a single bond bonded to L₂, one of R_{11A} to R_{15A}, R_{21A} to R_{27A}, R_{31A}, and R_{33A} to R_{37A} in L₂ is a single bond bonded to L₁;
when R₃₃ in L₁ is a single bond bonded to L₂, one of R_{11A} to R_{15A}, R_{21A} to R_{27A}, R_{31A} to R_{32A}, and R_{34A} to R_{37A} in L₂ is a single bond bonded to L₁;
when R₃₄ in L₁ is a single bond bonded to L₂, one of R_{11A} to R_{15A}, R_{21A} to R_{27A}, R_{31A} to R_{33A}, and R_{35A} to R_{37A} in L₂ is a single bond bonded to L₁;
when R₃₅ in L₁ is a single bond bonded to L₂, one of R_{11A} to R_{15A}, R_{21A} to R_{27A}, R_{31A} to R_{34A}, and R_{36A} to R_{37A} in L₂ is a single bond bonded to L₁;
when R₃₆ in L₁ is a single bond bonded to L₂, one of R_{11A} to R_{15A}, R_{21A} to R_{27A}, R_{31A} to R_{35A}, and R_{37A} in L₂ is a single bond bonded to L₁; and
when R₃₇ in L₁ is a single bond bonded to L₂, one of R_{11A} to R_{15A}, R_{21A} to R_{27A}, and R_{31A} to R_{36A} in L₂ is a single bond bonded to L₁.

7. The compound according to claim 6, wherein
-L₁-L₂- represents a group represented by one of the following formulae (13-1) to (13-69), 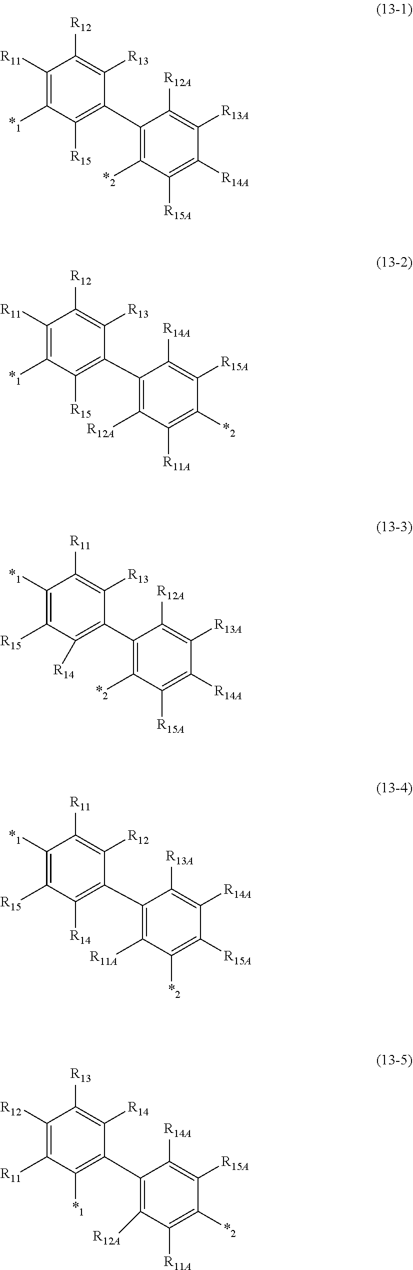 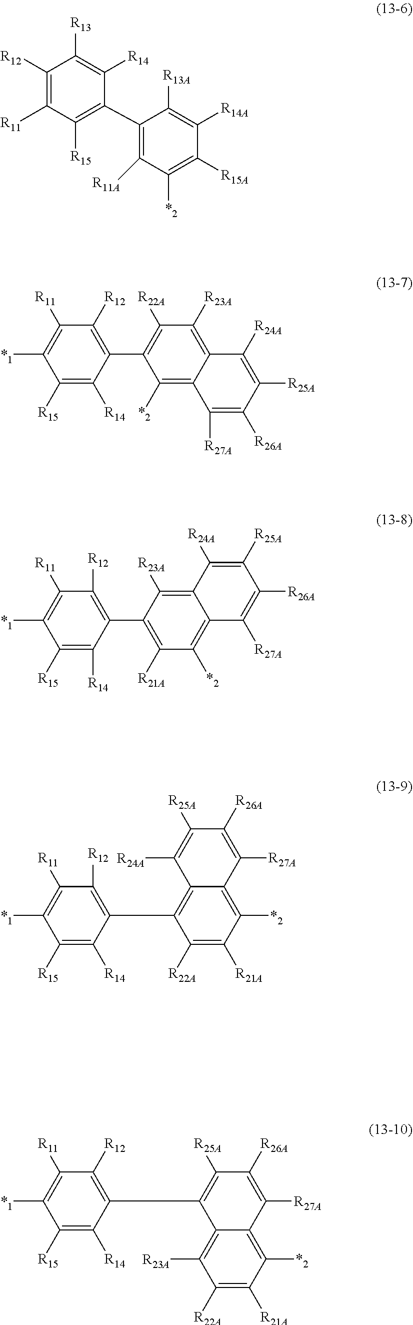 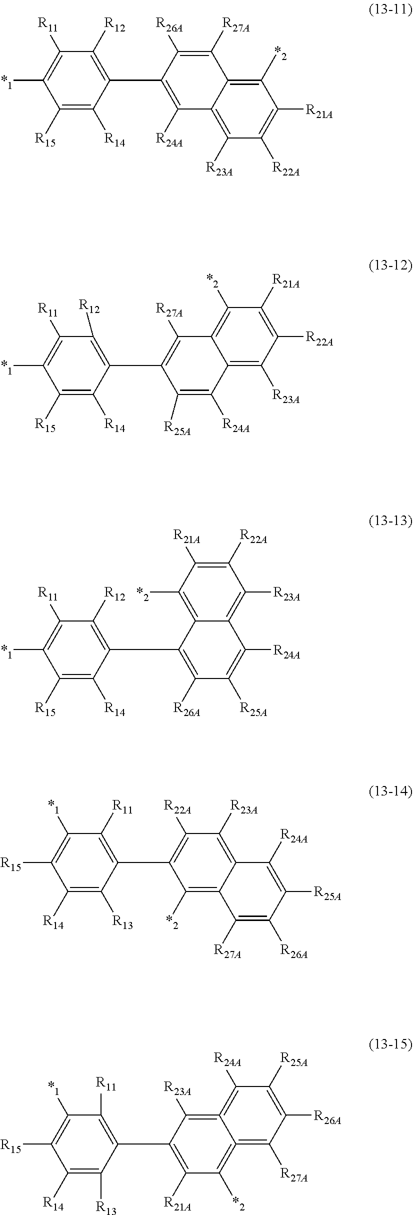 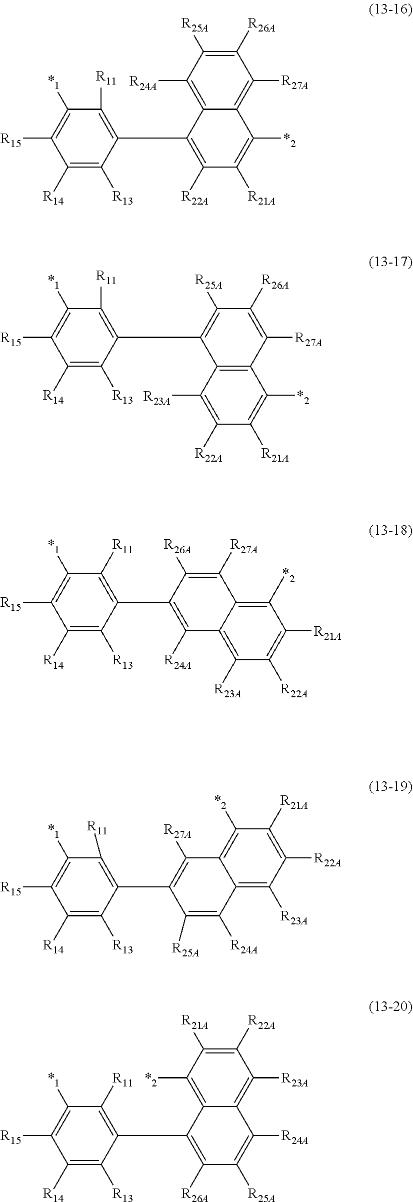 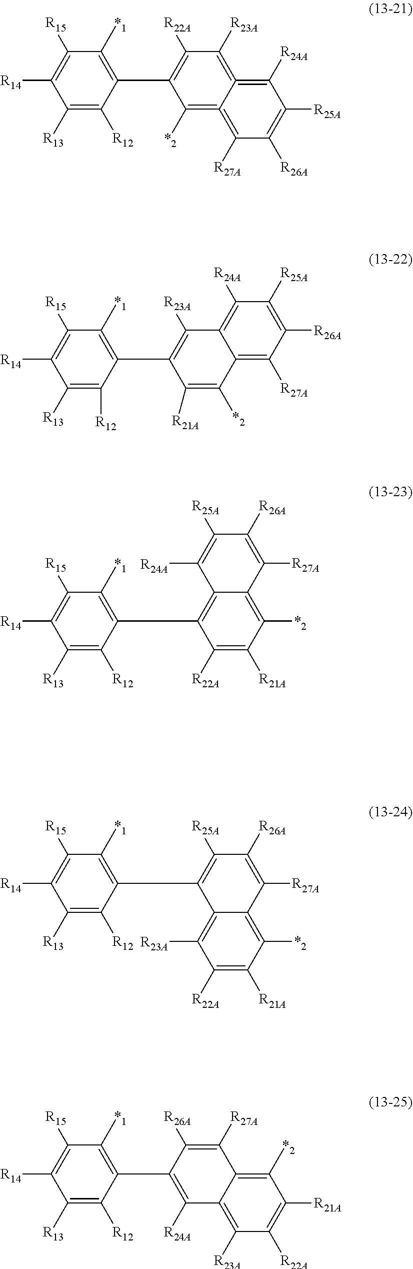 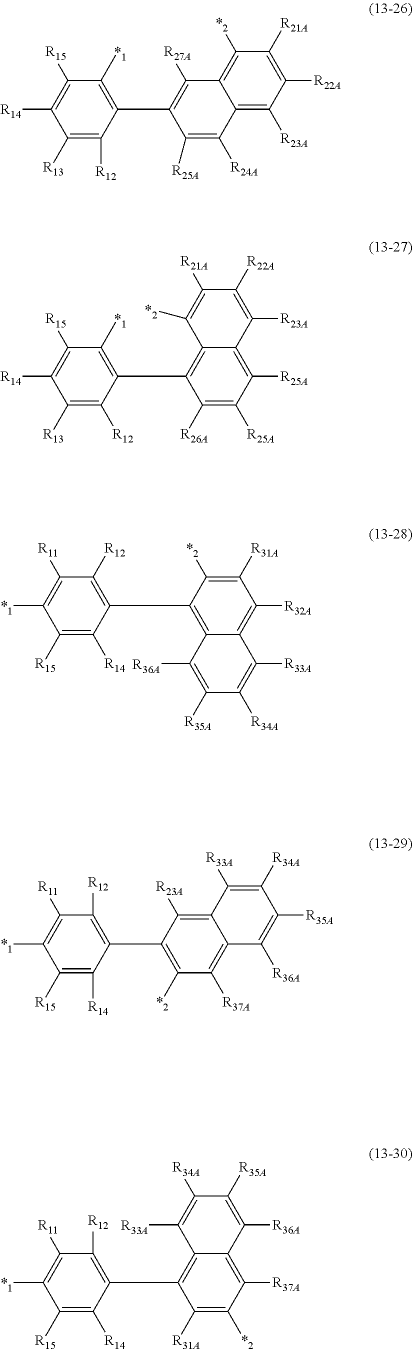 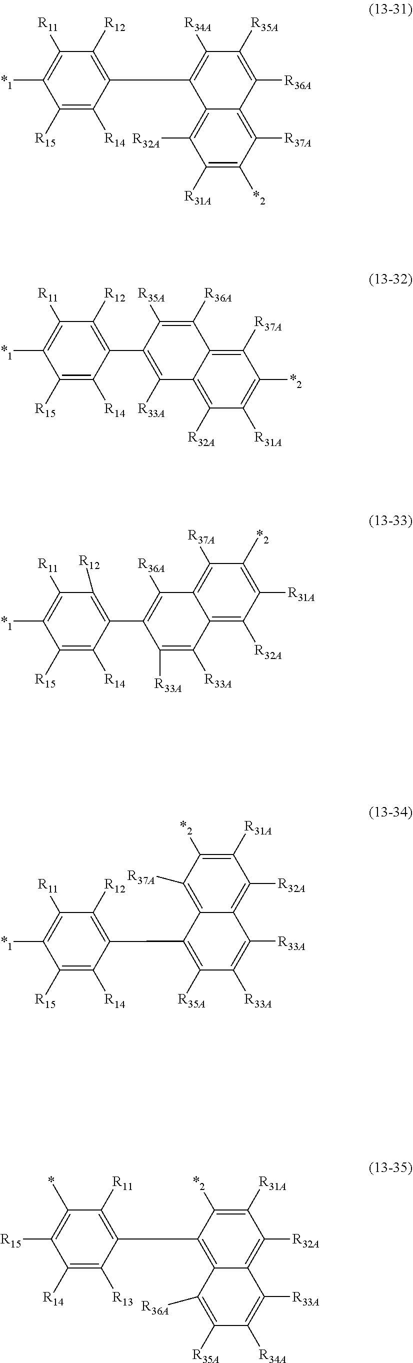 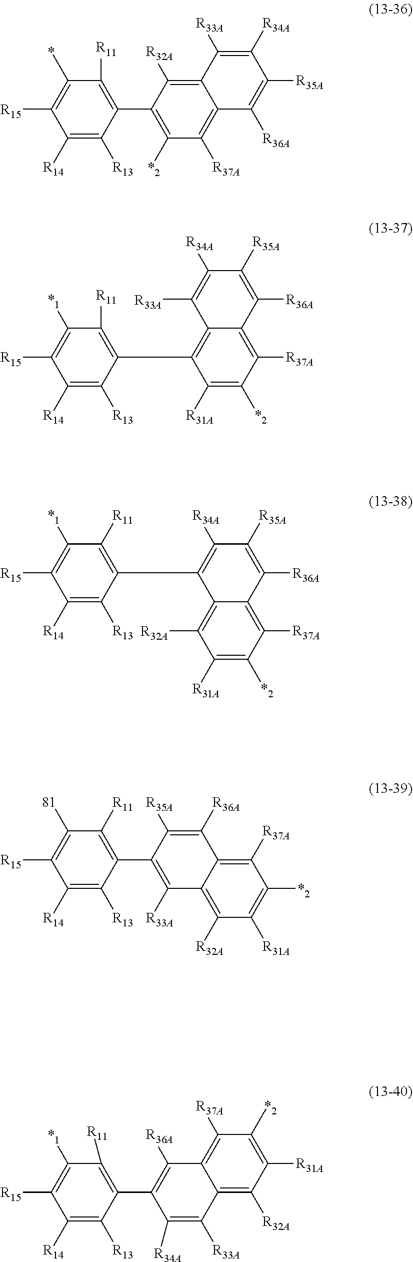 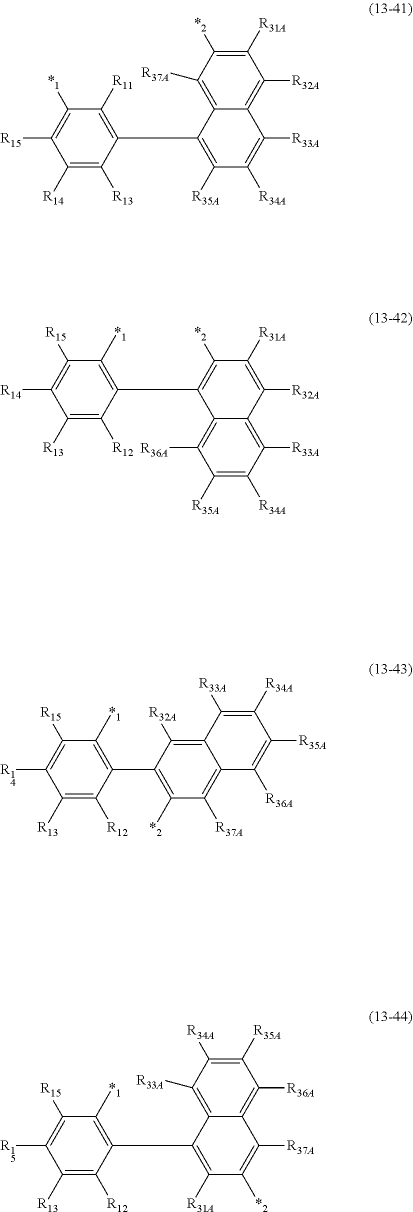 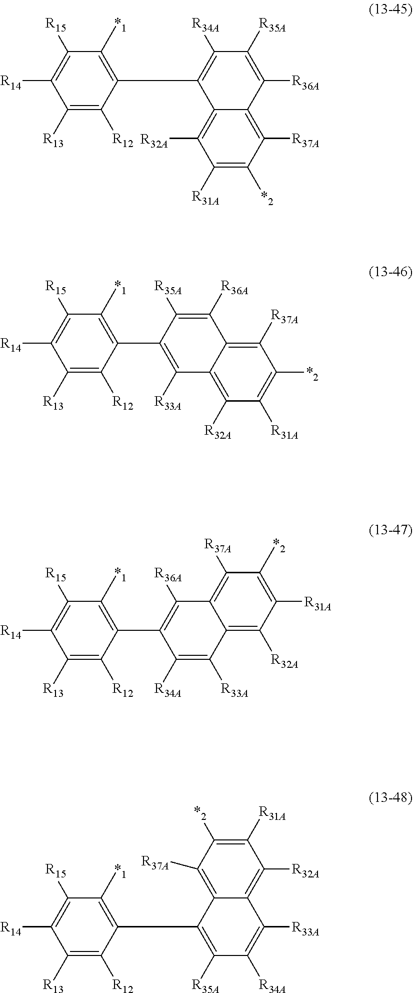 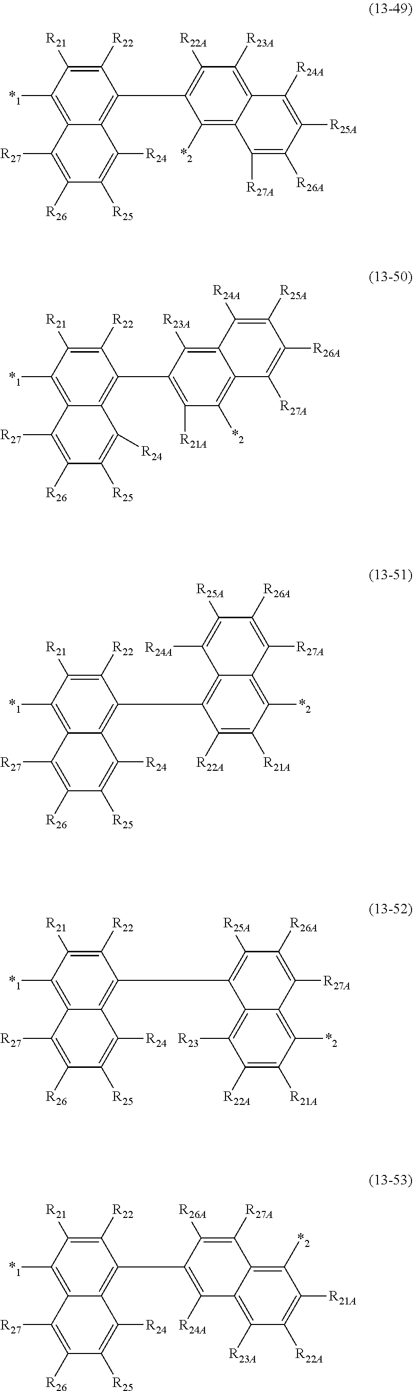 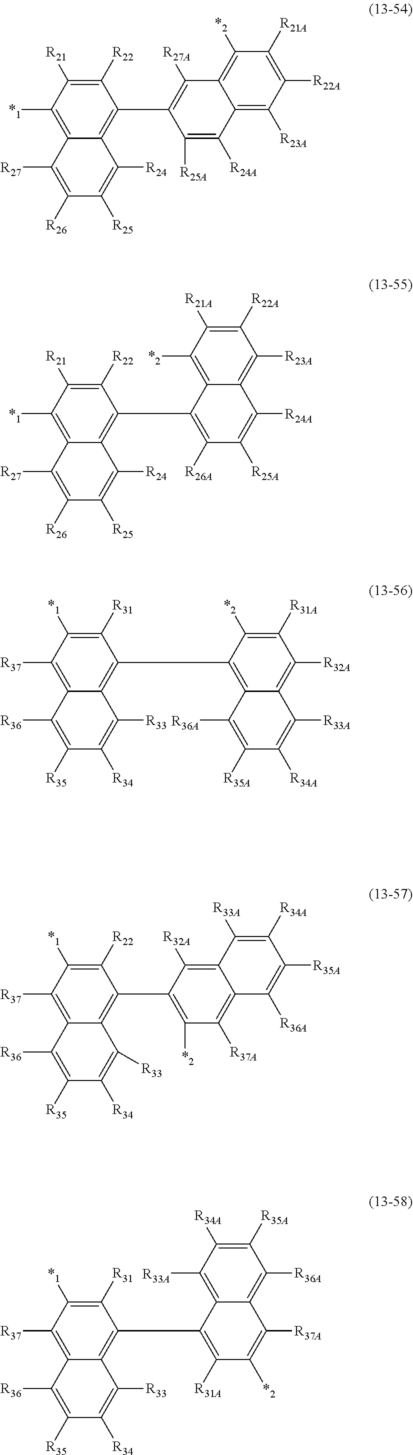 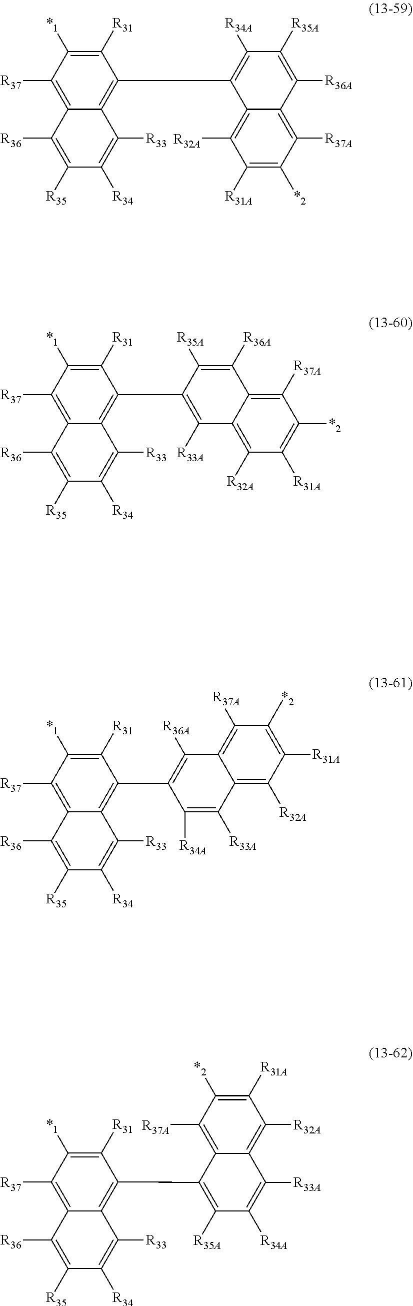 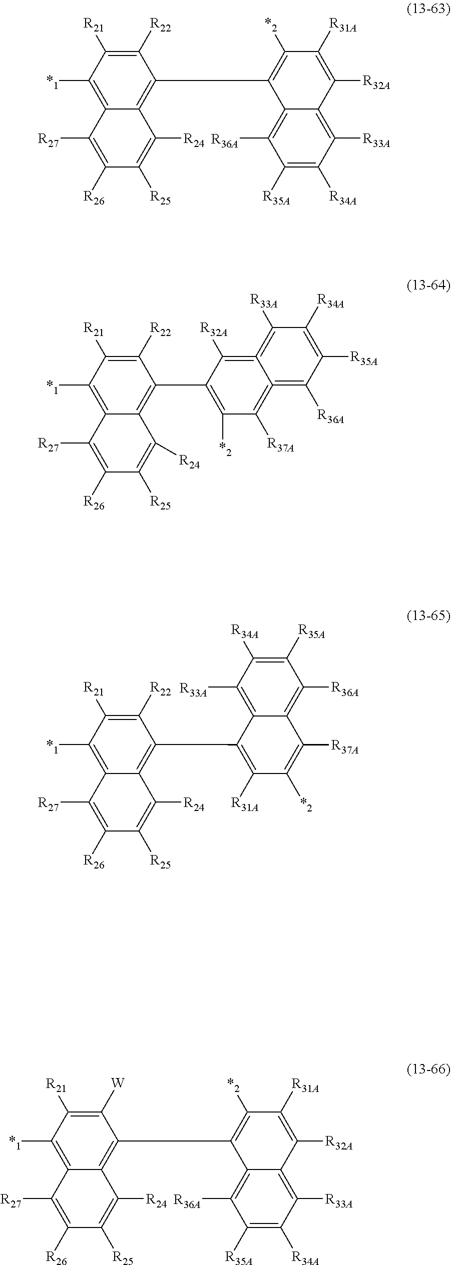 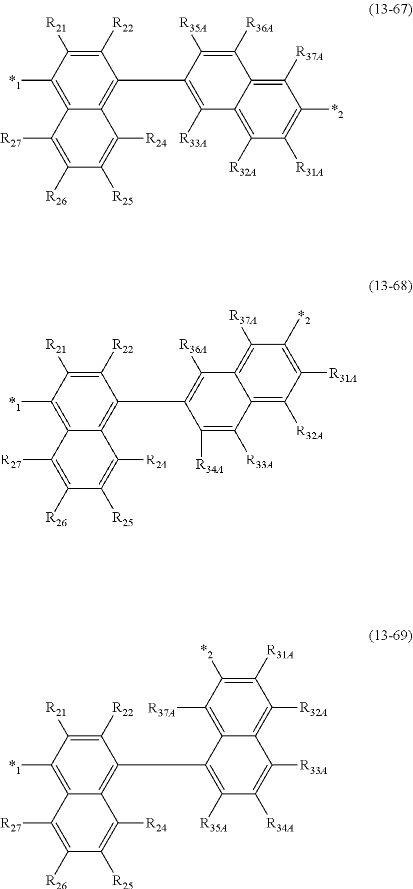
in the formulae (13-1) to (13-69):
R₁₁ to R₁₅, R₂₁ to R₂₇, R₃₁ to R₃₇, R_{11A} to R_{15A}, R_{21A} to R_{27A}, and R_{31A} to R_{37A} represent the same as R₁₁ to R₁₅, R₂₁ to R₂₇, R₃₁ to R₃₇, R_{11A} to R_{15A}, R_{21A} to R_{27A}, and R_{31A} to R_{37A}, respectively, in the formulae (11) to (13) and (11A) to (13A);
*1 in the formulae (13-1) to (13-69) represents a bonding position to *a in the formula (120); and
*2 represents a position of bonding to *b in the formula (120))

8. The compound according to claim 6, wherein
a compound represented by the formula (120) is represented by one of formulae (121) to (131) below, 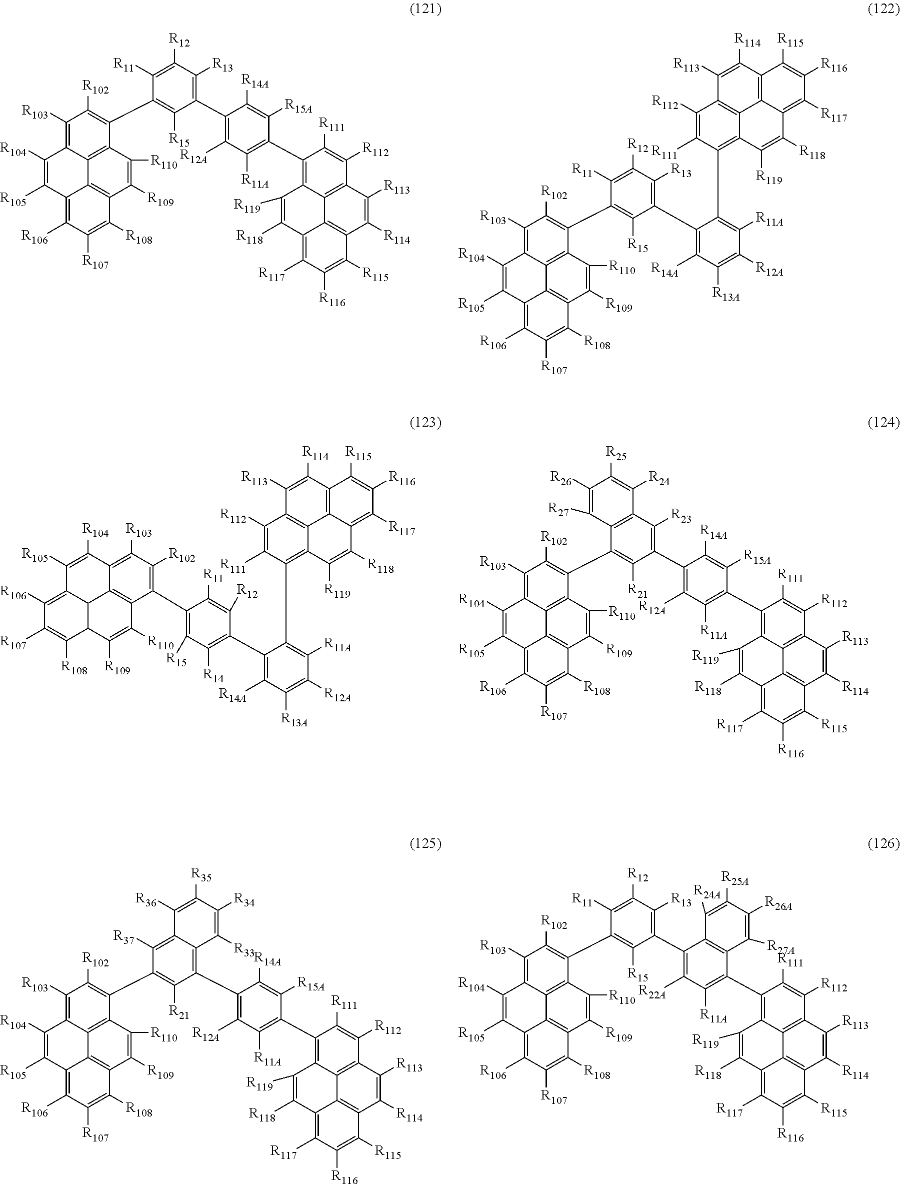 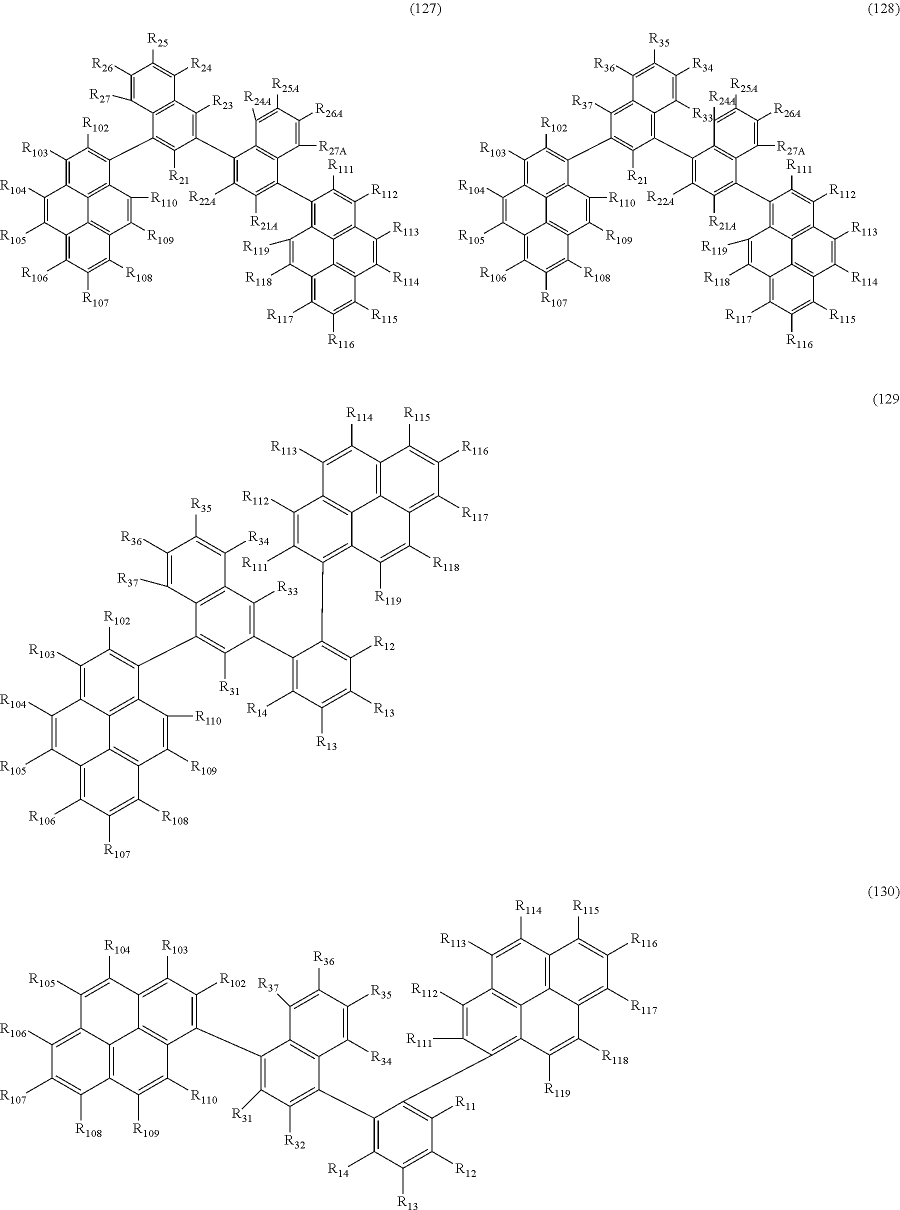 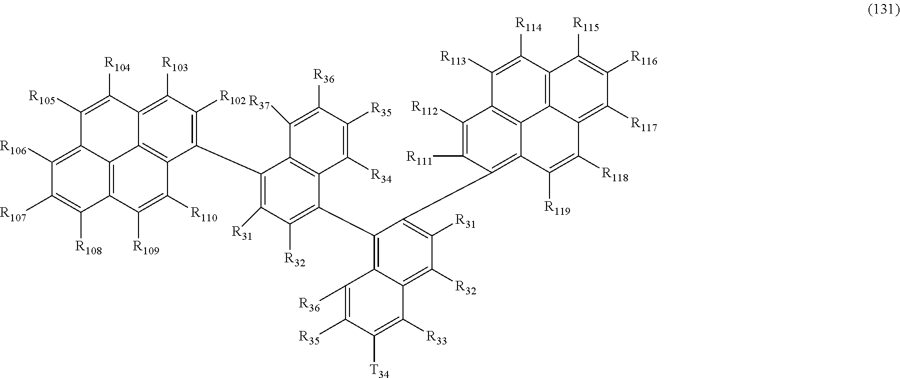
in the formula (121) to (131):
R₁₁ to R₁₅, R₂₁ to R₂₇, R₃₁ to R₃₇, R_{11A} to R_{15A}, R_{21A} to R_{27A}, and R_{31A} to R_{37A} represent the same as R₁₁ to R₁₅, R₂₁ to R₂₇, R₃₁ to R₃₇, R_{11A} to R_{15A}, R_{21A} to R_{27A}, and R_{31A} to R_{37A}, respectively, in the formulae (11) to (13) and (11A) to (13A); and
R₁₀₂ to R₁₁₉ each independently represent the same as R₁₀₂ to R₁₁₉, respectively, in the formula (120).

9. The compound according to claim 6, wherein
R₁₁ to R₁₅, R₂₁ to R₂₇, R₃₁ to R₃₇, R_{11A} to R_{15A}, R_{21A} to R_{27A}, R_{31A} to R_{37A}, and R₁₀₂ to R₁₁₉ each independently represent a hydrogen atom, a substituted or unsubstituted alkyl group having 1 to 30 carbon atoms, a substituted or unsubstituted cycloalkyl group having 1 to 30 carbon atoms, a group represented by —Si(Rx)(Ry)(Rz), a substituted or unsubstituted aryl group having 6 to 30 ring carbon atoms, or a substituted or unsubstituted heterocyclic group having 5 to 30 ring atoms,
Rx, Ry, and Rz in —Si(Rx)(Ry)(Rz) each independently represent an unsubstituted alkyl group having 1 to 30 carbon atoms, or an unsubstituted aryl group having 6 to 30 ring carbon atoms.

10-14. (canceled)

15. An organic electroluminescence device comprising:
an anode;
a cathode; and
a first emitting layer between the anode and the cathode,
wherein the first emitting layer comprises the compound according to claim 6 as a first host material.

16. The organic electroluminescence device according to claim 15, wherein
the first emitting layer further comprises a fluorescent compound.

17. The organic electroluminescence device according to claim 16, wherein
a singlet energy S₁ of the first host material is larger than a singlet energy S₁ of the fluorescent compound.

18. The organic electroluminescence device according to claim 15, wherein
the first emitting layer does not contain a metal complex.

19-21. (canceled)

22. A compound represented by a formula (1), (2) or (3), 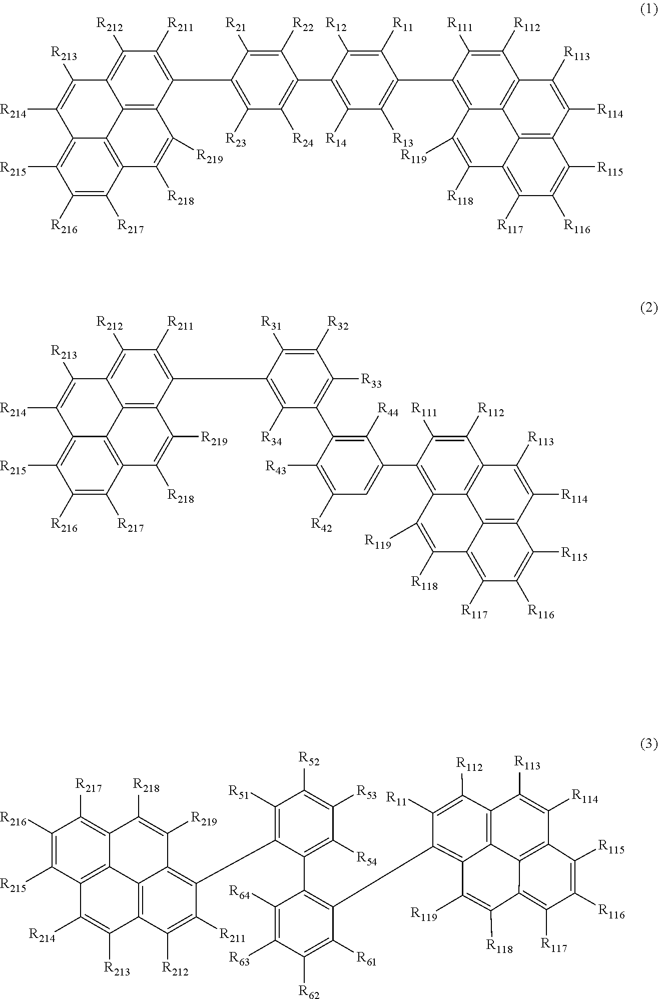
where: R₁₁₁ to R₁₁₉ and R₂₁₁ to R₂₁₉ each independently represent a hydrogen atom, a substituted or unsubstituted alkyl group having 1 to 50 carbon atoms, a substituted or unsubstituted cycloalkyl group having 3 to 50 carbon atoms, a group represented by —Si(Rx)(Ry)(Rz), a substituted or unsubstituted aryl group having 6 to 50 ring carbon atoms, or a substituted or unsubstituted heterocyclic group having 5 to 50 ring atoms,
Rx, Ry, and Rz in —Si(Rx)(Ry)(Rz) each independently represent a substituted or unsubstituted alkyl group having 1 to 50 carbon atoms, or a substituted or unsubstituted aryl group having 6 to 50 ring carbon atoms,
in the formula (1),
at least one combination of a combination of R₁₁ and R₁₂, a combination of R₁₃ and R₁₄, a combination of R₂₁ and R₂₂, or a combination of R₂₃ and R₂₄ are mutually bonded to form a substituted or unsubstituted monocyclic ring, are mutually bonded to form a substituted or unsubstituted fused ring, or are not mutually bonded,
R₁₁, R₁₃, R₂₁, and R₂₃ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4),
R₁₂, R₁₄, R₂₂, and R₂₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, or a group represented by the following formula (4); and
at least one of R₁₁ to R₁₄ or R₂₁ to R₂₄ is not a hydrogen atom,
in the formulae (2) to (3),
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring, are mutually bonded to form a substituted or unsubstituted fused ring, or are not mutually bonded;
R₃₁ to R₃₄, R₄₁ to R₄₄, R₅₁ to R₅₄, and R₆₁ to R₆₄ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted spirobifluorenyl group, or a group represented by the following formula (4);
at least one of R₃₁ to R₃₄ or R₄₁ to R₄₄ is not a hydrogen atom, and
at least one of R₅₁ to R₅₄ or R₆₁ to R₆₄ is not a hydrogen atom,
in a compound represented by the formula (1), a combination of R₁₁ and R₁₃ and a combination of R₂₁ and R₂₃ are different combinations, or a combination of R₁₂ and R₁₄ and a combination of R₂₂ and R₂₄ are different combinations;
in a compound represented by the formula (2), a combination of R₃₁ and R₄₁ is different from at least one combination of a combination of R₃₂ and R₄₂, a combination of R₃₃ and R₄₃, or a combination of R₃₄ and R₄₄; and
in a compound represented by the formula (3), a combination of R₅₁ and R₆₁ is different from at least one combination of a combination of R₅₂ and R₆₂, a combination of R₅₃ and R₆₃, or a combination of R₅₄ and R₆₄, 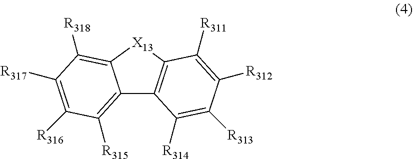
where: X₁₃ represents an oxygen atom, a sulfur atom, or NR₃₁₉,
at least one combination of adjacent two or more of R₃₁₁ to R₃₁₈ are mutually bonded to form a substituted or unsubstituted monocyclic ring, are mutually bonded to form a substituted or unsubstituted fused ring, or are not mutually bonded;
R₃₁₁ to R₃₁₉ that do not form the substituted or unsubstituted monocyclic ring and do not form the substituted or unsubstituted fused ring each independently represent a hydrogen atom, a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group; and
any one of R₃₁₁ to R₃₁₉ that do not form the substituted or unsubstituted monocyclic ring and do not form the substituted or unsubstituted fused ring is a single bond bonded to at least one of R₁₁ to R₁₄, R₂₁ to R₂₄, R₃₁ to R₃₄, R₄₁ to R₄₄, R₅₁ to R₅₄, or R₆₁ to R₆₄, in the formulae (1) to (3):
a substituent for a substituted or unsubstituted group in R₁₁ to R₁₄, R₂₁ to R₂₄, R₃₁ to R₃₄, R₄₁ to R₄₄, R₅₁ to R₅₄, R₆₁ to R₆₄, R₁₁₁ to R₁₁₉, R₂₁₁ to R₂₁₉, and R₃₁₁ to R₃₁₉ is each independently a substituted or unsubstituted alkyl group having 1 to 50 carbon atoms, a substituted or unsubstituted heterocyclic group having 5 to 50 ring atoms, a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted phenanthryl group, a substituted or unsubstituted chrysenyl group, a substituted or unsubstituted triphenylenyl group, a substituted or unsubstituted fluorenyl group, a substituted or unsubstituted 9,9′-spirobifluorenyl group, a substituted or unsubstituted 9,9-dimethylfluorenyl group, or a substituted or unsubstituted 9,9-diphenylfluorenyl group.

23-24. (canceled)

25. The compound according to claim 22,
represented by a formula (1-1), (2-1), or (3-1) below, 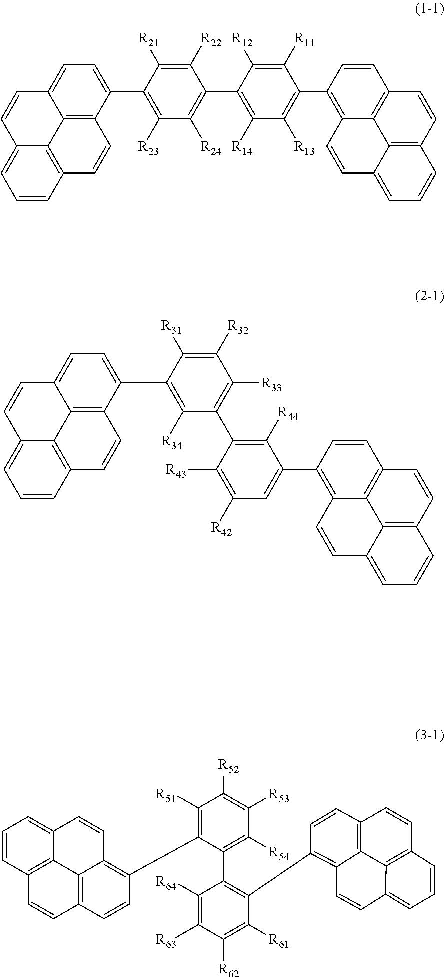
where: R₁₁ to R₁₄, R₂₁ to R₂₄, R₃₁ to R₃₄, R₄₁ to R₄₄, R₅₁ to R₅₄, and R₆₁ to R₆₄ represent the same as R₁₁ to R₁₄, R₂₁ to R₂₄, R₃₁ to R₃₄, R₄₁ to R₄₄, R₅₁ to R₅₄, and R₆₁ to R₆₄, respectively, in the formulae (1) to (3).

26. The compound according to claim 22, wherein
R₁₁ to R₁₄, R₂₁ to R₂₄, R₃₁ to R₃₄, R₄₁ to R₄₄, R₅₁ to R₅₄, and R₆₁ to R₆₄ each independently represent a hydrogen atom, a substituted or unsubstituted alkyl group having 1 to 8 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, a substituted or unsubstituted 9,9-dimethylfluorenyl group, a substituted or unsubstituted 9,9-diphenylfluorenyl group, a substituted or unsubstituted dibenzofuranyl group, or a substituted or unsubstituted dibenzothiophenyl group; and
R₁₂, R₁₄, R₂₂, and R₂₄ each are not a substituted or unsubstituted phenyl group.

27. The compound according to claim 22, wherein
at least one combination of a combination of R₁₁ and R₁₂, a combination of R₁₃ and R₁₄, a combination of R₂₁ and R₂₂, or a combination of R₂₃ and R₂₄ are mutually bonded to form a substituted or unsubstituted monocyclic ring or a substituted or unsubstituted fused ring, and
at least one combination of adjacent two or more of R₃₁ to R₃₃, R₄₁ to R₄₃, R₅₁ to R₅₄, and R₆₁ to R₆₄ are mutually bonded to form a substituted or unsubstituted monocyclic ring or a substituted or unsubstituted fused ring.

28. The compound according to claim 27,
represented by one of formulae (1-2), (2-2) to (2-3), and (3-2) to (3-4) below, 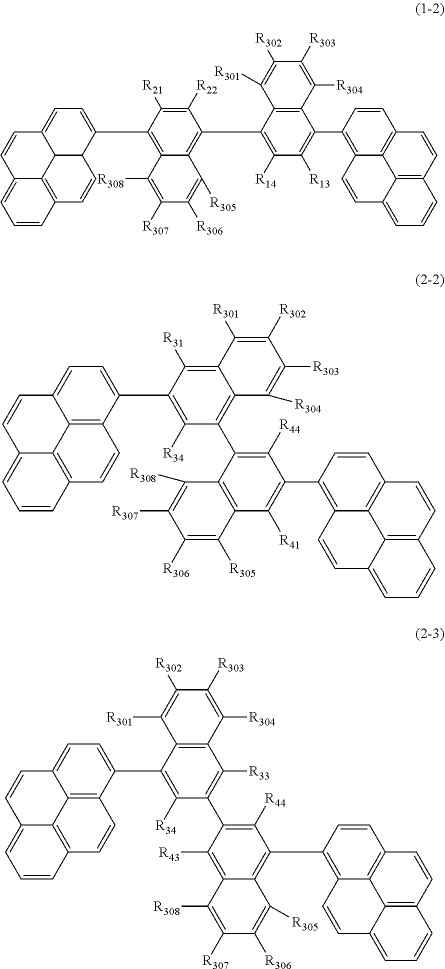 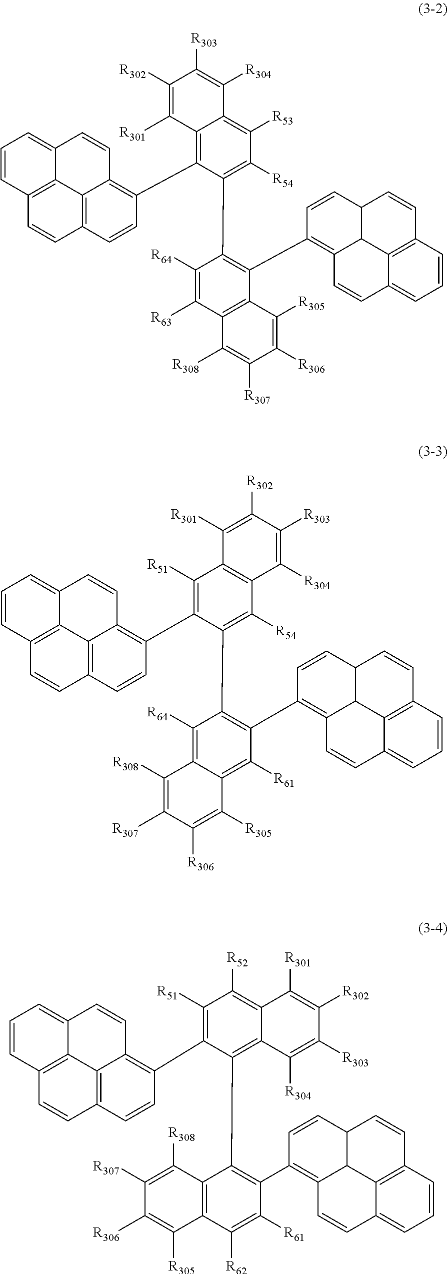
where: R₁₃ to R₁₄, R₂₁ to R₂₂, R₃₁, R₃₃, R₃₄, R₄₁, R₄₃, R₄₄, R₅₁ to R₅₄, and R₆₁ to R₆₄ represent the same as R₁₃ to R₁₄, R₂₁ to R₂₂, R₃₁, R₃₃, R₃₄, R₄₁, R₄₃, R₄₄, R₅₁ to R₅₄, and R₆₁ to R₆₄, respectively, in the formulae (1) to (3); and
R₃₀₁ to R₃₀₈ each independently represent the same as R₁₁ to R₁₄, R₂₁ to R₂₄, R₃₁ to R₃₄, R₄₁ to R₄₄, R₅₁ to R₅₄, and R₆₁ to R₆₄ in the formulae (1) to (3).

29. The compound according to claim 28, wherein
R₁₃ to R₁₄, R₂₁ to R₂₂, R₃₁, R₃₃, R₃₄, R₄₁, R₄₃, R₄₄, R₅₁ to R₅₄, R₆₁ to R₆₄, and R₃₀₁ to R₃₀₈ each independently represent a hydrogen atom, a substituted or unsubstituted alkyl group having 1 to 8 carbon atoms, a substituted or unsubstituted phenyl group, a substituted or unsubstituted biphenyl group, a substituted or unsubstituted naphthyl group, a substituted or unsubstituted phenanthryl group, a substituted or unsubstituted fluorenyl group, a substituted or unsubstituted 9,9′-spirobifluorenyl group, a substituted or unsubstituted 9,9-dimethylfluorenyl group, a substituted or unsubstituted 9,9-diphenylfluorenyl group, a substituted or unsubstituted dibenzofuranyl group, or a substituted or unsubstituted dibenzothiophenyl group; and
R₁₄ and R₂₂ each are not a substituted or unsubstituted phenyl group.

30. (canceled)

31. An organic electroluminescence device comprising:
an anode;
a cathode;
a first emitting layer between the anode and the cathode,
wherein the first emitting layer comprises the compound according to claim 22 as a first host material.

32. The organic electroluminescence device according to claim 31, wherein
the first emitting layer further comprises a fluorescent compound.

33. The organic electroluminescence device according to claim 32, wherein
a singlet energy S₁ of the first host material is larger than a singlet energy S₁ of the fluorescent compound,
S₁(M2)>S₁(M1) (Numerical Formula 1).

34. The organic electroluminescence device according to claim 31, wherein
the first emitting layer does not contain a metal complex.

35-37. (canceled)

38. The organic electroluminescence device according to claim 15, further comprising:
a second emitting layer between the first emitting layer and the cathode.

39. The organic electroluminescence device according to claim 38, wherein
the second emitting layer comprises a second compound represented by a formula (2) below as a second host material, 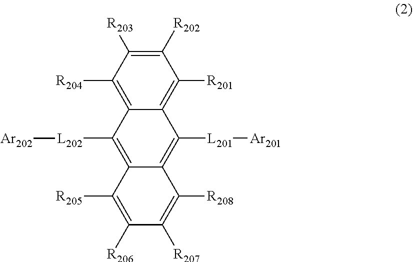
where:
R₂₀₁ to R₂₀₈ each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms,
L₂₀₁ and L₂₀₂ each independently represent a single bond, a substituted or unsubstituted arylene group having 6 to 50 ring carbon atoms, or a substituted or unsubstituted divalent heterocyclic group having 5 to 50 ring atoms, and
Ar₂₀₁ and Ar₂₀₂ each independently represent a substituted or unsubstituted aryl group having 6 to 50 ring carbon atoms, or a substituted or unsubstituted heterocyclic group having 5 to 50 ring atoms,
in the second compound, R₉₀₁, R₉₀₂, R₉₀₃, R₉₀₄, R₉₀₅, R₉₀₆, R₉₀₇, R₈₀₁, and R₈₀₂ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,
when a plurality of R₉₀₁ are present, the plurality of R₉₀₁ are mutually the same or different,
when a plurality of R₉₀₂ are present, the plurality of R₉₀₂ are mutually the same or different,
when a plurality of R₉₀₃ are present, the plurality of R₉₀₃ are mutually the same or different,
when a plurality of R₉₀₄ are present, the plurality of R₉₀₄ are mutually the same or different,
when a plurality of R₉₀₅ are present, the plurality of R₉₀₅ are mutually the same or different,
when a plurality of R₉₀₆ are present, the plurality of R₉₀₆ are mutually the same or different,
when a plurality of R₉₀₇ are present, the plurality of R₉₀₇ are mutually the same or different,
when a plurality of R₈₀₁ are present, the plurality of R₈₀₁ are mutually the same or different, and
when a plurality of R₈₀₂ are present, the plurality of R₈₀₂ are mutually the same or different.

40. The organic electroluminescence device according to claim 22, further comprising:
a second emitting layer between the first emitting layer and the cathode.

41. The organic electroluminescence device according to claim 40, wherein
the second emitting layer comprises a second compound represented by a formula (2) below as a second host material, 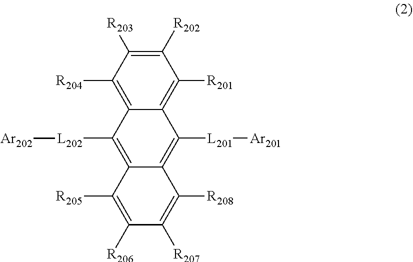
where:
R₂₀₁ to R₂₀₈ each independently represent a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms,
L₂₀₁ and L₂₀₂ each independently represent a single bond, a substituted or unsubstituted arylene group having 6 to 50 ring carbon atoms, or a substituted or unsubstituted divalent heterocyclic group having 5 to 50 ring atoms, and
Ar₂₀₁ and Ar₂₀₂ each independently represent a substituted or unsubstituted aryl group having 6 to 50 ring carbon atoms, or a substituted or unsubstituted heterocyclic group having 5 to 50 ring atoms,
in the second compound, R₉₀₁, R₉₀₂, R₉₀₃, R₉₀₄, R₉₀₅, R₉₀₆, R₉₀₇, R₈₀₁, and R₈₀₂ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,
when a plurality of R₉₀₁ are present, the plurality of R₉₀₁ are mutually the same or different,
when a plurality of R₉₀₂ are present, the plurality of R₉₀₂ are mutually the same or different,
when a plurality of R₉₀₃ are present, the plurality of R₉₀₃ are mutually the same or different,
when a plurality of R₉₀₄ are present, the plurality of R₉₀₄ are mutually the same or different,
when a plurality of R₉₀₅ are present, the plurality of R₉₀₅ are mutually the same or different,
when a plurality of R₉₀₆ are present, the plurality of R₉₀₆ are mutually the same or different,
when a plurality of R₉₀₇ are present, the plurality of R₉₀₇ are mutually the same or different,
when a plurality of R₈₀₁ are present, the plurality of R₈₀₁ are mutually the same or different, and
when a plurality of R₀₂ are present, the plurality of R₈₀₂ are mutually the same or different.
